(19) United States
(12) Patent Application Publication
(10) Pub. No.: US 2025/0257061 A1
(43) Pub. Date: Aug. 14, 2025

(54) COMPOSITIONS AND METHODS FOR TREATING CANCER AND REDUCING WNT MEDIATED EFFECTS

(71) Applicant: StemSynergy Therapeutics, Inc., Miami, FL (US)

(72) Inventors: Darren ORTON, Nashville, TN (US); Dennis Liang Fei, Miami, FL (US); Mark Rex Spyvee, Durham, NH (US)

(21) Appl. No.: 19/043,235

(22) Filed: Jan. 31, 2025

Related U.S. Application Data
(60) Provisional application No. 63/549,375, filed on Feb. 2, 2024.
(60) Provisional application No. 63/549,384, filed on Feb. 2, 2024.
(60) Provisional application No. 63/549,388, filed on Feb. 2, 2024.

(51) Int. Cl. C07D 417/14; A61K 31/4439; A61K 31/496; A61K 31/5377

(52) U.S. Cl. CPC C07D 417/14; A61K 31/4439; A61K 31/496; A61K 31/5377

(57) ABSTRACT

Disclosed herein, inter alia, are compounds, pharmaceutical compositions, and methods of reducing Wnt-mediated effects and treating cancer.

### CROSS-REFERENCE TO RELATED APPLICATIONS

This application claims the benefit of U.S. Provisional Application No. 63/549,375 filed Feb. 2, 2024; U.S. Provisional Application No. 63/549,384 filed Feb. 2, 2024; and U.S. Provisional Application No. 63/549,388 filed Feb. 2, 2024, all of which are incorporated herein by reference in their entirety and for all purposes.

### BACKGROUND

The Wnt pathway is an evolutionarily conserved growth pathway in multicellular organisms that regulates animal development and plays critical roles in human disease.

Signaling through the Wnt pathway is regulated by secreted Wnt proteins, which act as morphogens to mediate 1) cell fate determination and differentiation required for establishing the body plan, neural patterning, and organogenesis, 2) cell motility and polarity, 3) cell proliferation and apoptosis, and 4) stem cell maintenance.

In Wnt signaling, the transcriptional coactivator, beta-catenin, is constitutively degraded in the absence of a Wnt signal thereby allowing a cell to maintain low cytoplasmic levels of beta-catenin and keeping the Wnt pathway in the “off” position. Degradation of beta-catenin requires its recruitment into a complex consisting primarily of Glycogen synthase kinase (Gsk3), Casein Kinase 1 (CK1), Protein phosphatase 2A (PP2A), Axin, and the tumor suppressor adenomatous polyposis coli (APC). Within this complex, beta-catenin is phosphorylated by CK1, which primes it for further phosphorylation by Gsk3. Phosphorylated beta-catenin is recognized by the SCF (Skip1, Cullen, F-box) ubiquitin ligase complex, of which the specificity F-box determinant is beta-TRCP, and targeted for polyubiquitination and subsequent degradation by the proteasome. The Wnt pathway is turned “on” upon binding of Wnt ligands to the Frizzled family of receptors and the coreceptor family members LDL receptor-related protein 5 or 6 (LRP5/6), which results in translocation of the beta-catenin destruction complex to the membrane through interaction of Axin with LRP5/6. The interaction between Axin and LRP5/6 is promoted by the phosphorylation of LRP5/6 by CK1 and Gsk3, and Axin-LRP5/6 interaction results in inhibition of beta-catenin phosphorylation and degradation. Because beta-catenin is continually synthesized in cells, its cytoplasmic concentration increases, and it enters the nucleus and forms a complex with the TCF/LEF1 family of transcriptional factors (as well as the nuclear proteins BCL9 and Pygopus) to regulate a Wnt-specific transcriptional program.

Our bodies are composed of numerous cell types specialized to perform specific functions. These specialized or differentiated cells are derived from a small group of stem and progenitor cells that have the capacity to divide asymmetrically, allowing them to regenerate themselves, and also giving rise to a daughter cell that can differentiate into cell types characteristic of various organs in our bodies. It is recognized that diseases like diabetes, Parkinson's disease, and heart disease are caused by death or dysfunction of differentiated cells in tissues where stem cells are limiting. These diseases may be caused by loss of stem cell activity and/or misregulation of critical signaling pathways in stem cells residing in tissues such as the pancreas, brain, and heart. The Wnt pathway is a key regulator of stem cell behavior and viability, and modulation of this pathway presents a method of treating diseases associated with dysfunctional stem cell activity. For example, activation of the Wnt pathway has been associated with heart failure, and inhibition of Wnt signaling has been shown to improve recovery after a heart attack in animal models. Thus, Wnt inhibitors could have broad applications in regenerative (stem cell) medicine for the treatment of major human diseases such as heart disease.

Cancer has been shown to be stem cell related disease, resulting from failure of cells to respond to normal cues to stop proliferating. Wnt signaling is also a critical pathway that drives the uncontrolled proliferation of many solid tumors in cancer stem cells (CSCs). Thus, therapies that down-regulate the activity of Wnt signaling, a fundamental pathway in CSCs, would be effective in the treatment of cancer. Such inhibitors would result in a long-term therapeutic benefit because the cells capable of repopulating the tumor would be killed. Most notably, there is clear evidence that colorectal cancer arises from mutations in the stem cell compartment, and it has been demonstrated that all major solid cancers in humans (e.g., melanoma, hepatocellular carcinoma, and breast cancer) have abnormal Wnt signaling. Thus, Wnt inhibitors may be useful in the treatment of most of the major solid cancers in humans.

Disclosed herein, inter alia, are solutions to these and other problems known in the art.

### BRIEF SUMMARY

In an aspect is provided a compound, or a pharmaceutically acceptable salt or tautomer thereof, having the formula:

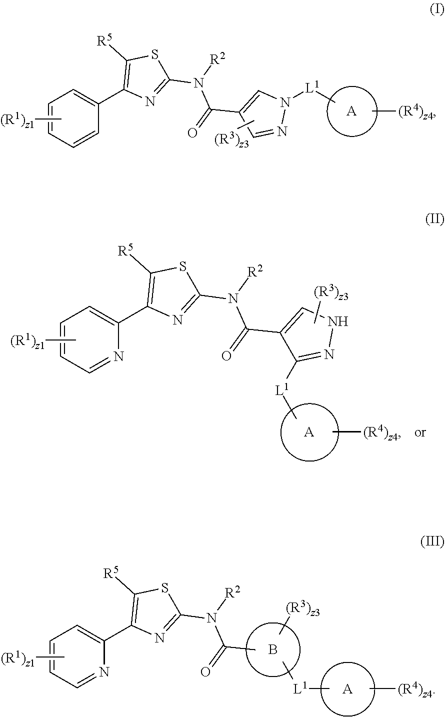

Ring A is cycloalkyl, heterocycloalkyl, aryl, or heteroaryl. Ring B is imidazolyl or triazolyl.

L¹ is a bond, substituted or unsubstituted alkylene, or substituted or unsubstituted heteroalkylene.

R¹ is independently halogen, —CX¹₃, —CHX¹₂, —CH₂X¹, —OCX¹₃, —OCH₂X¹, —OCHX¹₂, —CN, —SOₙ₁R^{1D}, —Soᵥ₁NR^{1A}R^{1B}, —NR^{1C}NR^{1A}R^{1B}, —ONR^{1A}R^{1B}, —NR^{1C}C(O)NR^{1A}R^{1B}—N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z1 is an integer from 0 to 4.

R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR³CC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z3 is an integer from 0 to 2.

R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z4 is an integer from 0 to 11.

R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl.

Each X¹, X³, and X⁴ is independently —F, —Cl, —Br, or —I. The symbols n1, n3, and n4 are independently an integer from 0 to 4. The symbols m1, m3, m4, v1, v3, and v4 are independently 1 or 2.

In an aspect is provided a compound, or a pharmaceutically acceptable salt or tautomer thereof, having the formula:

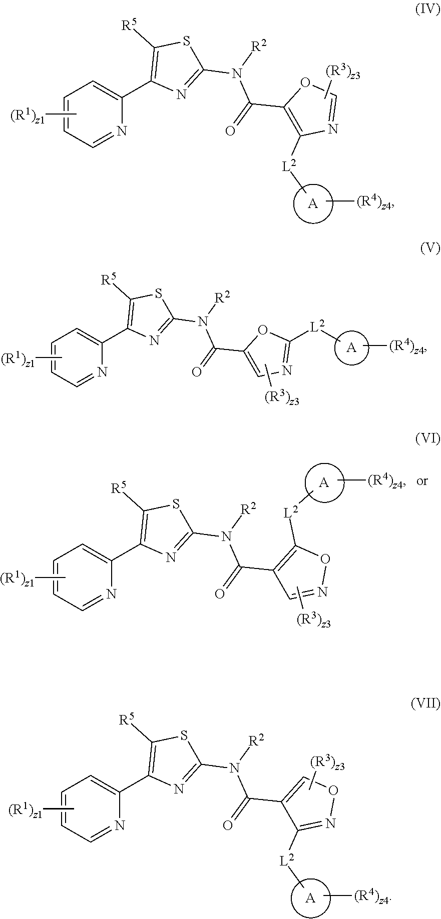

Ring A is cycloalkyl, heterocycloalkyl, aryl, or heteroaryl.

L² is a bond, —C(O)NR¹⁰—, —NR¹⁰C(O)—, —NR¹⁰S(O)₂—, —S(O)₂NR¹⁰—, substituted or unsubstituted alkylene, or substituted or unsubstituted heteroalkylene.

R¹⁰ is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z1 is an integer from 0 to 4.

R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is independently halogen, —CX³₃, —CHX³², —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR³CC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The symbol z3 is 0 or 1.

R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR⁴CO(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z4 is an integer from 0 to 11.

R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl.

Each X¹, X³, and X⁴ is independently —F, —Cl, —Br, or —I. The symbols n1, n3, and n4 are independently an integer from 0 to 4. The symbols m1, m3, m4, v1, v3, and v4 are independently 1 or 2.

In an aspect is provided a compound, or a pharmaceutically acceptable salt or tautomer thereof, having the formula:

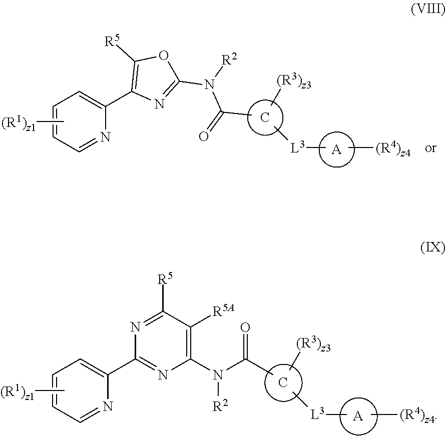

Ring A is cycloalkyl, heterocycloalkyl, aryl, or heteroaryl.

Ring C is pyrazolyl, oxazolyl, pyrrolyl, imidazolyl, triazolyl, or tetrazolyl.

L³ is a bond, —O—, substituted or unsubstituted alkylene, or substituted or unsubstituted heteroalkylene.

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z1 is an integer from 0 to 4.

R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR³CC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z3 is an integer from 0 to 2.

R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z4 is an integer from 0 to 11.

R⁵ and R^{5A} are independently hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ and R⁵ substituents may optionally be joined to form a substituted or unsubstituted unsubstituted aryl or substituted or unsubstituted heteroaryl.

R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl.

Each X¹, X³, and X⁴ is independently —F, —Cl, —Br, or —I. The symbols n1, n3, and n4 are independently an integer from 0 to 4. The symbols m1, m3, m4, v1, v3, and v4 are independently 1 or 2.

In an aspect is provided a compound, or a pharmaceutically acceptable salt or tautomer thereof, having the formula:

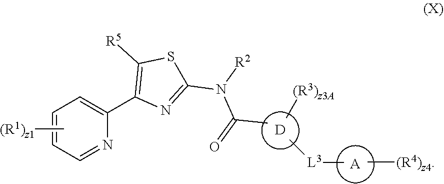

Ring A, L³, R¹, z1, R², R³, R⁴, z4, and R⁵ are as described herein, including in embodiments. Ring D is pyridonyl, pyrazinyl, pyridyl, or phenyl. The symbol z3A is an integer from 0 to 4.

In an aspect is provided a pharmaceutical composition including a compound described herein, or a pharmaceutically acceptable salt or tautomer thereof, and a pharmaceutically acceptable excipient.

In an aspect is provided a method of treating a cancer in a subject in need thereof, the method including administering to the subject in need thereof a therapeutically effective amount of a compound described herein, or a pharmaceutically acceptable salt or tautomer thereof.

In an aspect is provided a method of reducing a Wnt-mediated effect on a cell, the method including contacting the cell with an effective amount of a compound as described herein, or a pharmaceutically acceptable salt or tautomer thereof.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O—is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain.

Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

[symbol]

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

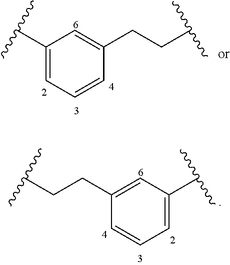 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂ CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′, —NR″C(O)₂R′, —NRC(NR′R″R″′)═NR″″, —NRC(NR′R″)═NR″′, —S(O)R′, —S(O)₂R′, —S(O)₂NR′R″, —NRSO₂R′, 5, —NR′NR″R″′, —ONR′R″, —NR′C(O)NR″NR″′R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula —T—C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)₅—X′—(C″R″R″′)_{d}—, where s and d are independently integers of from 0 to 3, and X is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₅ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₅ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₅ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHN₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₅ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₅ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R¹, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.2}, and the like up to or exceeding an R¹⁰° that may be substituted with one or more first substituent groups denoted by R^{100.1} As a further example, R^{1A} may be substituted with one or more first substituent groups denoted by R^{1A.2}, R^{2A} may be substituted with one or more first substituent groups denoted by R^{2A.1}, R^{3A} may be substituted with one or more first substituent groups denoted by R^{3A.1}, R⁴A may be substituted with one or more first substituent groups denoted by R^{4A.2}, R^{5A} may be substituted with one or more first substituent groups denoted by R^{5A.1} and the like up to or exceeding an R^{100A} may be substituted with one or more first substituent groups denoted by R^{100A.1} As a further example, L¹ may be substituted with one or more first substituent groups denoted by R^{L1.1}, L² may be substituted with one or more first substituent groups denoted by R^{L20.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g. R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{Ll00.1}) may be further substituted with one or more second substituent groups (e.g. R^{1.1}, R^{2.1}, R^{3.1}, R^{4.1}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{WW.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g. R^{1.2}, R^{2.2}, R^{3.2}, R^{4.2}, R^{5.2}R^{100.2}; R^{1A.2}R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g. R^{1.2}, R^{2.3}, R^{3.2}, R^{4.2}, R^{5.1} . . . R^{100.1}; R^{1A.3}, R^{2A.3}, R^{3A.3}, R^{4A.3}, R^{5A.3} . . . R^{100A.3}; R^{L.3}R^{L2.3}, R^{L3.3}, R^{L4.3}R^{L5.3} . . . R^{L1.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g. when R^{WW.1} is R^{WW.2} substituted alkyl, examples of groups so formed include but are not limited to itself optionally substituted by 1 or more R^{WW.2}, which R^{WW.2} is optionally substituted by one or more R^{WW.3}. By way of example when R^{WW.1} is alkyl, groups that could be formed, include but are not limited to:

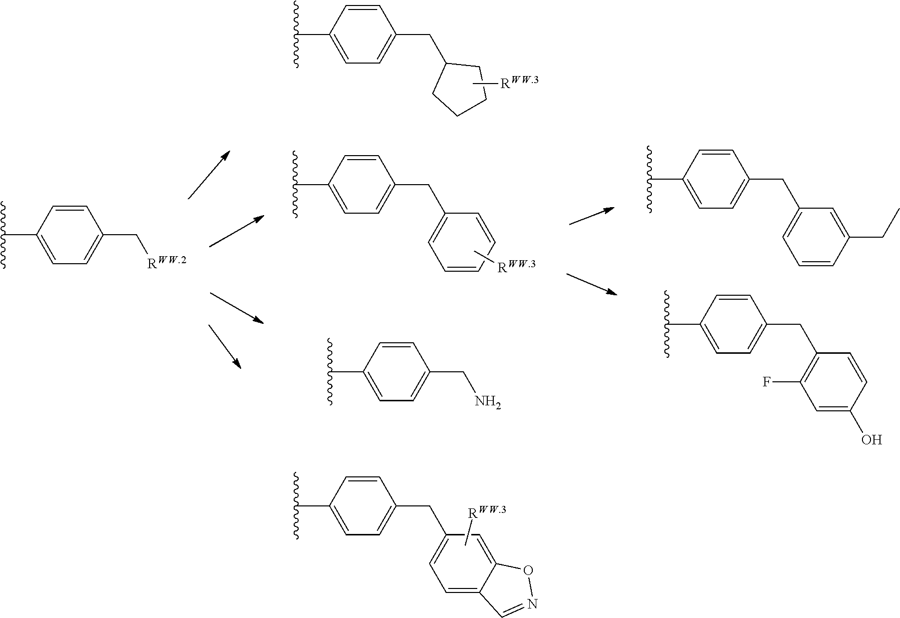

R^{WW.1} is independently oxo, halogen, —CX^{WW.1}₃, —CHX^{WW.1}₂, —CH₂X^{WW.1}, —OCX^{WW.1}₃ —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NH—NH₂, —NHC(O)NH₂, —NHC(NH)NH₂, —NHSO₂H, —NHC(O)H, —NHC(O)OH, —NHOH, —N₃, unsubstituted alkyl (e.g., C₁-C₅, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃—OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₅, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.22}, —CH₂X^{WW.2}, —OCX^{WW.2}₃, —OCH₂X^{WW.2}, —OCHX^{WW.22}, —CN, —OH, —NH₂, —COOH, —CONH₂, —NO₂, —SH, —SO₃H, —OSO₃H, —SO₂NH₂, —NHNH₂, —ONH₂, —NHC(O)NH—NH₂, —NHC(O)NH₂, —NHC(NH)NH₂, —NHSO₂H, —NHC(O)H, —NHC(O)OH, —NHOH, —N₃, unsubstituted alkyl (e.g., C₁-C₅, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₅, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW2}; and each second substituent group, R^{WW2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments 25 where R^{100A} and R^{100B} are optionally joined together to form an openly substituted ring, R^{WW.1} is R^{100B.1}, R^{WW.2} is R^{100B.2}, and R^{WW.3} is R^{100B.3}, R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₅, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1} —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₅, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.23}, —CHX^{LWW.22}, —CH₂X^{LWW.2}, —OCX^{LWW.23}, —OCH₂X^{LWW.2}, —OCHX^{LWW.22}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₅, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered).

In embodiments, R^{LWW.2} is independently oxo, halogen, —CX^{LWW.23}, —CHX^{LWW.22}, —CH₂X^{LWW.2}, —OCX^{LWW.23}, —OCH₂X^{LWW.2}, —OCHX^{LWW.22}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₅, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₅, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW3}, —CHX^{WW2}, —CH₂X^{WW}, —OCX^{WW}₃, —OCH₂X^{WW}, —OCHX^{WW2}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₅, C₁-C₆, C₁-C₄, or C₁-C₂), R^{WW}.-substituted or unsubstituted heteroalkyl (e.g., 2 to 8 membered, 2 to 6 membered, 4 to 6 membered, 2 to 3 membered, or 4 to 5 membered), R^{WW.1}—substituted or unsubstituted cycloalkyl (e.g., C₃-C₈, C₃-C₆, C₄-C₆, or C₅-C₆), R^{WW.1}—substituted or unsubstituted heterocycloalkyl (e.g., 3 to 8 membered, 3 to 6 membered, 4 to 6 membered, 4 to 5 membered, or 5 to 6 membered), R^{WW.1}—substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₅, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)—, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ^{1.3}C— or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (^{1.25}I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R^{1.3} substituents are present, each R^{1.3} substituent may be distinguished as R^{1.3A}R^{1.3.B}, R^{1.3.C}, R^{1.3.D} etc., wherein each of R^{1.3A}, R^{1.3.B}, R^{1.3.C}, R^{1.3.D}, etc. is defined within the scope of the definition of R^{1.3} and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds disclosed herein can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a cancer.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs.

Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²p, ³³p, ⁴⁵Ti, ⁴⁷Sc, ⁵²Fe, ⁵⁹Fe, ⁶²Cu, ⁶⁴CU, ⁶⁷Cu, ⁶⁷Ga, ⁶⁸Ga, ⁷⁷As, ⁸⁶y, ⁹⁰y, ⁸⁹Sr, ⁸⁹Zr, ⁹⁴TC, ⁹⁴TC, ⁹⁹mTc, ⁹⁹Mo, ¹⁰⁵Pd, ¹⁰⁵Rh, ¹¹¹Ag, ¹¹¹In, ^{1.23}J, ^{1.24}J, ^{1.25}J, ^{1.31}J, ¹⁴²Pr, ¹⁴³Pr, ¹⁴⁹Pm, ¹⁵³Sm, ¹⁵⁴⁻¹⁵⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴r ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³₂P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²p, ³₃p, ⁴⁵Ti, ⁴⁷SC, ⁵²Fe, ⁶²Cu, ⁶⁴Cu, ⁶⁷Cu, ⁶⁷Ga ⁶⁸Ga, ⁷⁷As, ⁸⁶y, ⁹⁰Y, ⁸⁹Sr, ⁸⁹Zr, ⁹⁴Tc, ⁹⁴Tc, ⁹⁹mTc, 99Mo, ¹⁰⁵Pd, ¹⁰⁵Rh, ¹¹¹Ag, ¹¹¹n, ^{1.23}I, ^{1.24}I, ^{1.25}I, ^{1.31}I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions disclosed herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds disclosed herein. One of skill in the art will recognize that other pharmaceutical excipients are useful in the presently disclosed pharmaceutical compositions.

The term “preparation” is intended to include the formulation of the active compound with encapsulating material as a carrier providing a capsule in which the active component with or without other carriers, is surrounded by a carrier, which is thus in association with it.

Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compounds disclosed herein can be administered alone or can be co-administered to the patient. Co-administration is meant to include simultaneous or sequential administration of the compounds individually or in combination (more than one compound).

Thus, the preparations can also be combined, when desired, with other active substances (e.g., to reduce metabolic degradation). The compositions disclosed herein can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound(s) utilized in the pharmaceutical compositions disclosed herein may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer) diagnosed in a particular patient. The dose administered to a patient, in the context of the presently disclosed methods of therapeutic treatment, should be sufficient to affect a beneficial therapeutic response in the patient over time.

The size of the dose will also be determined by the existence, nature, and extent of any adverse side effects that accompany the administration of a compound in a particular patient.

Determination of the proper dosage for a particular situation is within the skill of the practitioner.

Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). In embodiments, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates (also referred to herein as amorphous protein aggregates), oligomers (also referred to herein as protein oligomers), and amyloid fibrils.

### II. Compounds

In an aspect is provided a compound, or a pharmaceutically acceptable salt or tautomer thereof, having the formula:

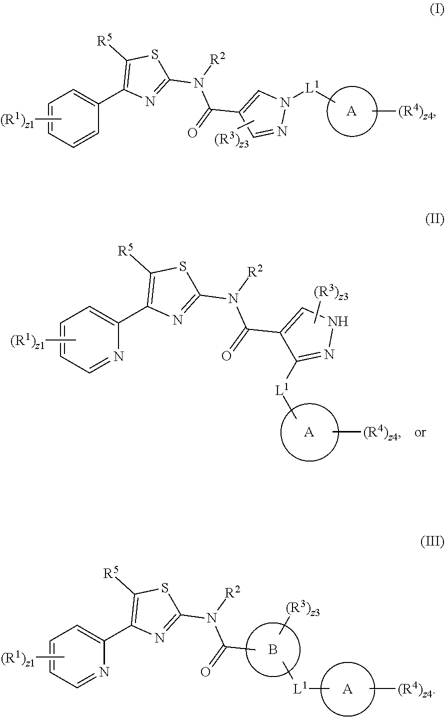

Ring A is cycloalkyl (e.g., C₃-C₈, C₃-C₆, or C₅-C_{G}), heterocycloalkyl (e.g., 3 to 8 membered, 3 to 6 membered, or 5 to 6 membered), aryl (e.g., C_{G}-C₁₀, C₁₀, or phenyl), or heteroaryl (e.g., 5 to 10 membered, 5 to 9 membered, or 5 to 6 membered).

Ring B is imidazolyl or triazolyl.

L¹ is a bond or substituted or unsubstituted alkylene (e.g., C₁-C₈, C₁-C_{G}, or C₁-C₄).

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR′CC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); two R¹ substituents may optionally be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z1 is an integer from 0 to 4.

R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR³CC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D} —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); two R³ substituents may optionally be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z3 is an integer from 0 to 2.

R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D},, —NR^{4A}SO₂R^{4D} 10, —NR^{4A}C(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); two R⁴ substituents may optionally be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z4 is an integer from 0 to 11.

R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered); R^{4A} and R^{4B}substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

Each X¹, X³, and X⁴ is independently —F, —Cl, —Br, or —I.

The symbols n1, n3, and n4 are independently an integer from 0 to 4.

The symbols m1, m3, m4, v1, v3, and v4 are independently 1 or 2.

In embodiments, the compound has the formula:

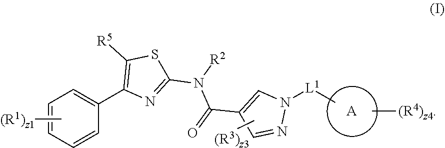

Ring A, L¹, R¹, z1, R², R³, z3, R⁴, z4, and R⁵ are as described herein, including in embodiments.

In embodiments, the compound has the formula:

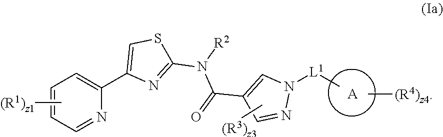

Ring A, L¹, R¹, z1, R², R³, z3, R⁴, and z4 are as described herein, including in embodiments.

In embodiments, the compound has the formula:

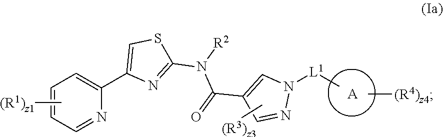

wherein Ring A is cycloalkyl, heterocycloalkyl, aryl, or heteroaryl; L¹ is a bond or substituted or unsubstituted alkylene; R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —C(O)NR^{1A}RB, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z1 is an integer from 0 to 4; R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}. —NR^{3C}NR^{3A}R^{3B}, —ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R³C, —NR^{3A}C(O)OR³C, —NR^{3A}OR³C, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 2; R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR⁴CNR^{4A}R^{4B}, —ONR^{4A}R^{4B}, —NR^{4C}C(O)NR^{4A}R^{4B}, —N(O)m4, —NR^{4A}R^{4B}, —C(O)R^{4C}, —C(O)OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl; z4 is an integer from 0 to 11; R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; each X¹, X³, and X⁴ is independently —F, —Cl, —Br, or —I; n1, n3, and n4 are independently an integer from 0 to 4; and m1, m3, m4, v1, v3, and v4 are independently 1 or 2.

In embodiments, R³ is not —CF₃, unsubstituted methyl, unsubstituted ethyl, unsubstituted cyclopropyl, substituted or unsubstituted phenyl, unsubstituted pyrrolyl, or unsubstituted thienyl. In embodiments, R³ is not —CX³₃, substituted or unsubstituted methyl, substituted or unsubstituted ethyl, substituted or unsubstituted cyclopropyl, substituted or unsubstituted phenyl, substituted or unsubstituted pyrrolyl, or substituted or unsubstituted thienyl.

In embodiments, when Ring A is phenyl, then z3 is not 0.

In embodiments, the compound has the formula:

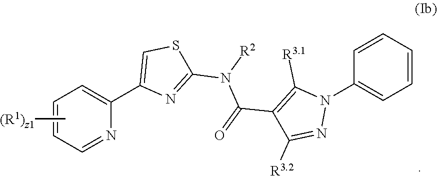

R¹, z1, and R² are as described herein, including in embodiments. R^{3.1} and R^{3.2} are independently any value of R³ as described herein, including in embodiments.

In embodiments, the compound has the formula:

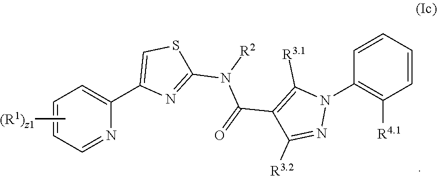

R¹, z1, and R² are as described herein, including in embodiments. R^{3.1} and R^{3.2} are independently any value of R³ as described herein, including in embodiments. R^{4.1} is any value of R⁴ as described herein, including in embodiments.

In embodiments, the compound has the formula:

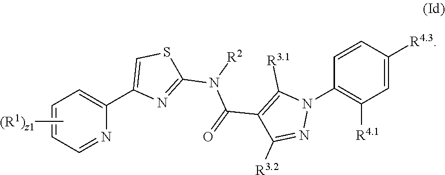

R¹, z1, and R² are as described herein, including in embodiments. R^{3.1} and R^{3.2} are independently any value of R³ as described herein, including in embodiments. R^{4.1} and R^{4.3} are independently any value of R⁴ as described herein, including in embodiments.

In embodiments, the compound has the formula:

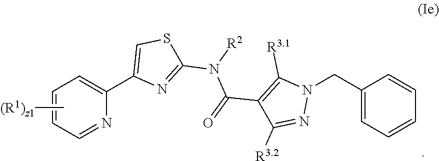

R¹, z1, and R² are as described herein, including in embodiments. R^{3.1} and R^{3.2} are independently any value of R³ as described herein, including in embodiments.

In embodiments, the compound has the formula:

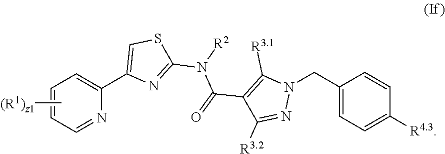

R¹, z1, and R² are as described herein, including in embodiments. R^{3.1} and R^{3.2} are independently any value of R³ as described herein, including in embodiments. R^{4.3} is any value of R⁴ as described herein, including in embodiments.

In embodiments, the compound has the formula:

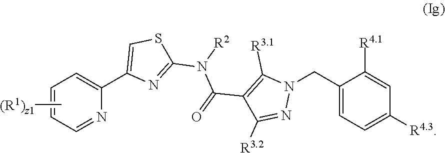

R¹, z1, and R² are as described herein, including in embodiments. R^{3.1} and R^{3.2} are independently any value of R³ as described herein, including in embodiments. R^{4.1} and R^{4.3} are independently any value of R⁴ as described herein, including in embodiments.

R^{3.1} and R^{3.2} are independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R³B —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C} —NR^{3A}C(O)OR^{3C}, —NR^{3A}OR^{3C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

R^{4.1} and R^{4.3} are independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D},, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In embodiments, the compound has the formula:

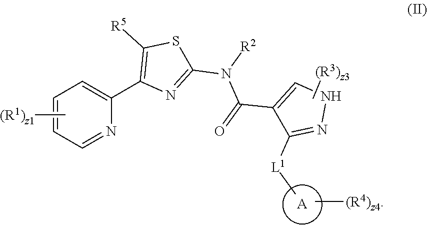

Ring A, L¹, R¹, z1, R², R³, z3, R⁴, z4, and R⁵ are as described herein, including in embodiments.

In embodiments, the compound has the formula:

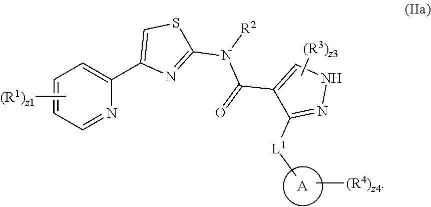

Ring A, L¹, R¹, z1, R², R³, z3, R⁴, and z4 are as described herein, including in embodiments.

In embodiments, Ring A is not cyclopropyl or pyrrolyl. In embodiments, R³ is not unsubstituted phenyl. In embodiments, R³ is not substituted or unsubstituted phenyl. In embodiments, when Ring A is phenyl, then R⁴ is not unsubstituted methoxy. In embodiments, when Ring A is phenyl, then z3 is not 0.

In embodiments, the compound has the formula:

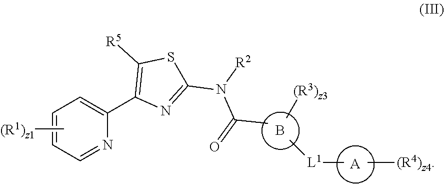

Ring A, Ring B, L¹, R¹, z1, R², R³, z3, R⁴, z4, and R⁵ are as described herein, including in embodiments.

In embodiments, the compound has the formula:

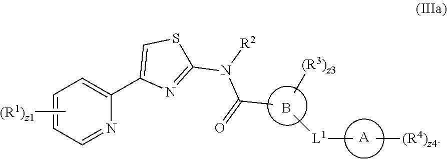

Ring A, Ring B, L¹, R¹, z1, R², R³, z3, R⁴, and z4 are as described herein, including in embodiments.

In embodiments, the compound has the formula:

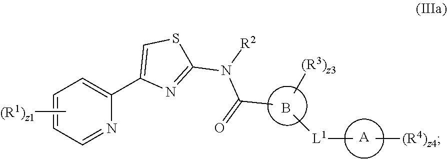

wherein Ring A is cycloalkyl, heterocycloalkyl, aryl, or heteroaryl; Ring B is imidazolyl or triazolyl; L¹ is a bond or substituted or unsubstituted alkylene; R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z1 is an integer from 0 to 4; R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR³CC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z3 is an integer from 0 to 2; R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, OR^{4D},, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl; z4 is an integer from 0 to 11; R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; each X¹, X³, and X⁴ is independently —F, —Cl, —Br, or —I; n1, n3, and n4 are independently an integer from 0 to 4; and m1, m3, m4, v1, v3, and v4 are independently 1 or 2.

In embodiments, when Ring A is phenyl, then z4 is not 0.

In embodiments, the compound has the formula:

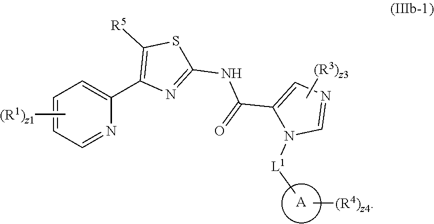

Ring A, L¹, R¹, z1, R³, z3, R⁴, z4, and R⁵ are as described herein, including in embodiments.

In embodiments, the compound has the formula:

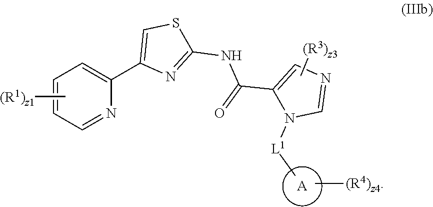

Ring A, L¹, R¹, z1, R³, z3, R⁴, and z4 are as described herein, including in embodiments.

In embodiments, the compound has the formula:

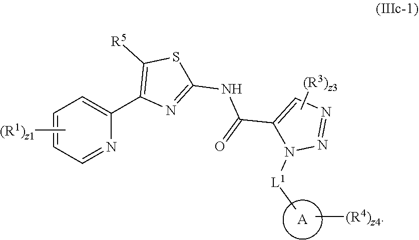

Ring A, L¹, R¹, z1, R³, z3, R⁴, z4, and R⁵ are as described herein, including in embodiments.

In embodiments, the compound has the formula:

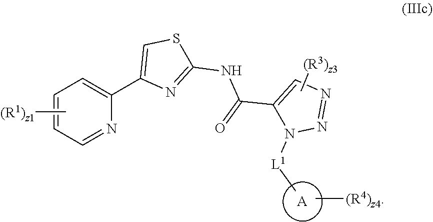

Ring A, L¹, R¹, z1, R³, z3, R⁴, and z4 are as described herein, including in embodiments.

In embodiments, the compound has the formula:

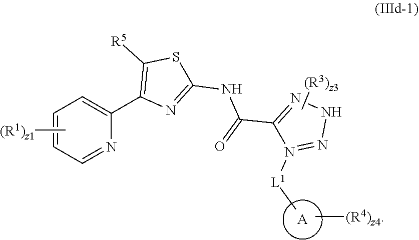

Ring A, L¹, R¹, z1, R³, z3, R⁴, z4, and R⁵ are as described herein, including in embodiments.

In embodiments, the compound has the formula:

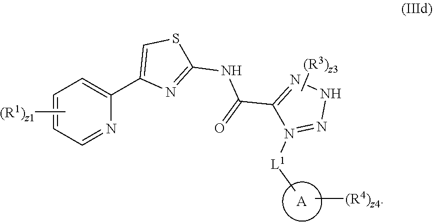

Ring A, L¹, R¹, z1, R³, z3, R⁴, and z4 are as described herein, including in embodiments.

In an aspect is provided a compound, or a pharmaceutically acceptable salt or tautomer thereof, having the formula:

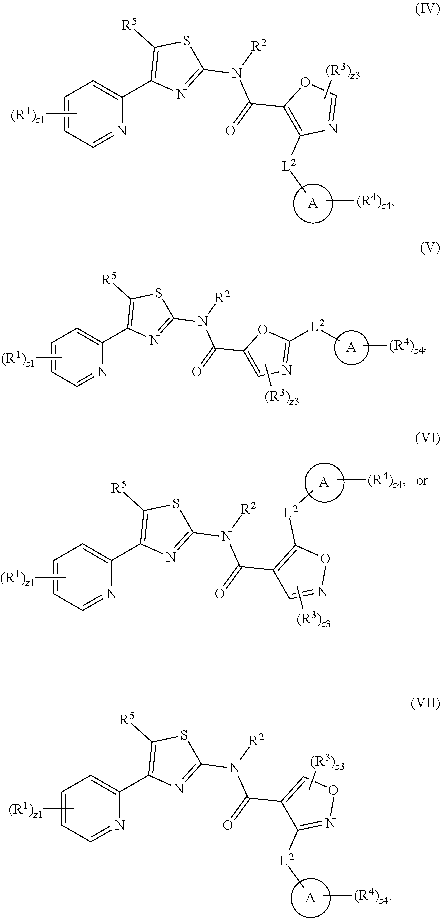

Ring A is cycloalkyl (e.g., C₃-C₈, C₃-C₆, or C₅-C₆), heterocycloalkyl (e.g., 3 to 8 membered, 3 to 6 membered, or 5 to 6 membered), aryl (e.g., C₆-C₁₀, C₁₀, or phenyl), or heteroaryl (e.g., 5 to 10 membered, 5 to 9 membered, or 5 to 6 membered).

L² is a bond, —C(O)NR¹⁰ —, —NR¹⁰, C(O)—, —NR¹⁰S(O)₂—, —S(O)₂NR¹⁰—, substituted or unsubstituted alkylene (e.g., C₁-C₅, C₁-C₆, or C₁-C₄), or substituted or unsubstituted heteroalkylene (e.g., 2 to 8 membered, 2 to 6 membered, or 2 to 4 membered).

R¹⁰ is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₅, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₅, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); two R¹ substituents may optionally be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z1 is an integer from 0 to 4.

R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR³CC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C} —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z3 is 0 or 1.

R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D},, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); two R⁴ substituents may optionally be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z4 is an integer from 0 to 11.

R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered); R^{4A} and R^{4B}substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

Each X¹, X³, and X⁴ is independently —F, —Cl, —Br, or —I.

The symbols n1, n3, and n4 are independently an integer from 0 to 4.

The symbols m1, m3, m4, v1, v3, and v4 are independently 1 or 2.

In embodiments, the compound has the formula:

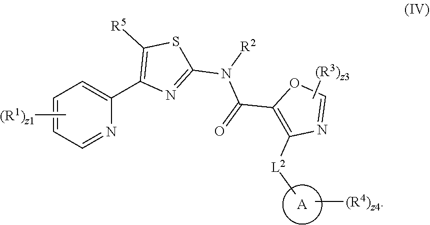

Ring A, L², R¹, z1, R², R³, z3, R⁴, z4, and R⁵ are as described herein, including in embodiments.

In embodiments, the compound has the formula:

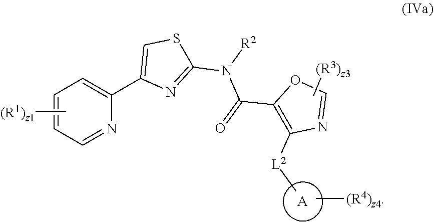

Ring A, L², R¹, z1, R², R³, z3, R⁴, and z4 are as described herein, including in embodiments.

In embodiments, the compound has the formula:

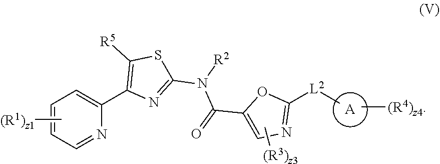

Ring A, L², R¹, z1, R², R³, z3, R⁴, z4, and R⁵ are as described herein, including in embodiments.

In embodiments, the compound has the formula:

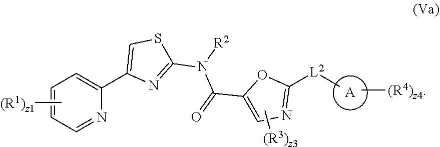

King A, L², R¹, z1, R², R³, z3, R⁴, and z4 are as described herein, including in embodiments.

In embodiments, the compound has the formula:

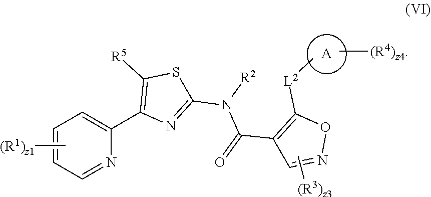

Ring A, L², R¹, z1, R², R³, z3, R⁴, z4, and R⁵ are as described herein, including in embodiments.

In embodiments, the compound has the formula:

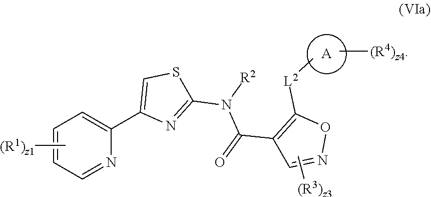

Ring A, L², R¹, z1, R², R³, z3, R⁴, and z4 are as described herein, including in embodiments.

In embodiments, the compound has the formula:

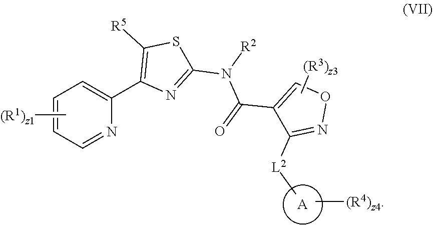

Ring A, L², R¹, z1, R², R³, z3, R⁴, z4, and R⁵ are as described herein, including in embodiments.

In embodiments, the compound has the formula:

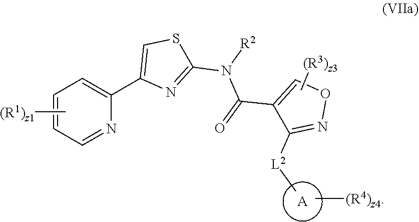

Ring A, L², R¹, z1, R², R³, z3, R⁴, and z4 are as described herein, including in embodiments.

In an aspect is provided a compound, or a pharmaceutically acceptable salt or tautomer thereof, having the formula:

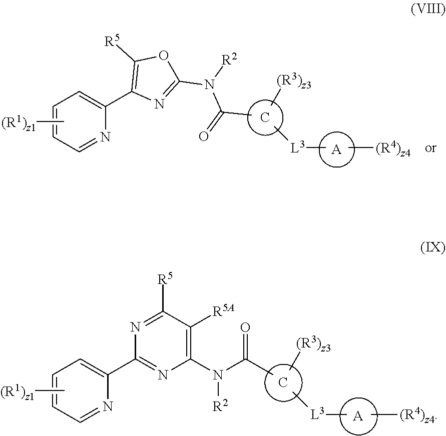

Ring A is cycloalkyl (e.g., C₃-C₈, C₃-C₆, or C₅-C₆), heterocycloalkyl (e.g., 3 to 8 membered, 3 to 6 membered, or 5 to 6 membered), aryl (e.g., C₆-C₁₀, C₁₀, or phenyl), or heteroaryl (e.g., 5 to 10 membered, 5 to 9 membered, or 5 to 6 membered).

Ring C is pyrazolyl, oxazolyl, pyrrolyl, imidazolyl, triazolyl, or tetrazolyl.

L³ is a bond, —O—, substituted or unsubstituted alkylene (e.g., C₁-C₅, C₁-C₆, or C₁-C₄), or substituted or unsubstituted heteroalkylene (e.g., 2 to 8 membered, 2 to 6 membered, or 2 to 4 membered).

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₅, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); two R¹ substituents may optionally be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z1 is an integer from 0 to 4.

R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₅, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR³CC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D} —NR³ASO₂R^{3D}, —NR^{3A}C(O)R^{3C}, —NR^{3A}C(O)OR^{3C} —NR^{3A}OR^{3C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); two R³ substituents may optionally be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z3 is an integer from 0 to 2.

R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D},, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); two R⁴ substituents may optionally be joined to form a substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

The symbol z4 is an integer from 0 to 11.

R⁵ and R^{5A} are independently hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl (e.g., C₁-C₅, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

R¹ and R⁵ substituents may optionally be joined to form a substituted or unsubstituted unsubstituted aryl (e.g., C₆-C₁₀, C₁₀, or phenyl) or substituted or unsubstituted heteroaryl (e.g., 5 to 10 membered, 5 to 9 membered, or 5 to 6 membered).

R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered); R⁴a and R^{4B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

Each X¹, X³, and X⁴ is independently —F, —Cl, —Br, or —I.

The symbols n1, n3, and n4 are independently an integer from 0 to 4.

The symbols m1, m3, m4, v1, v3, and v4 are independently 1 or 2.

In embodiments, the compound has the formula:

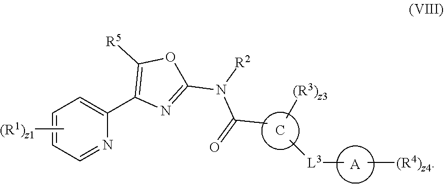

Ring A, Ring C, L³, R¹, z1, R², R³, z3, R⁴, z4, and R⁵ are as described herein, including in embodiments.

In embodiments, the compound has the formula:

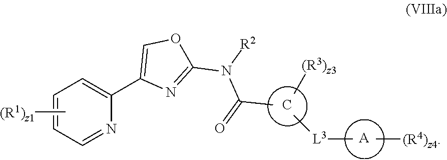

Ring A, Ring C, L³, R¹, z1, R², R³, z3, R⁴, and z4 are as described herein, including in embodiments.

In embodiments, the compound has the formula:

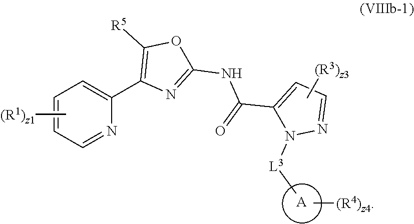

Ring A, L³, R¹, z1, R³, z3, R⁴, z4, and R⁵ are as described herein, including in embodiments.

In embodiments, the compound has the formula:

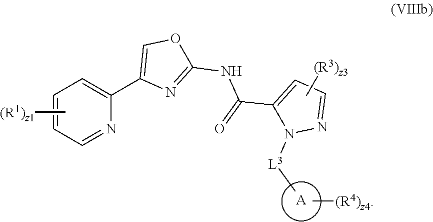

Ring A, L³, R¹, z1, R³, z3, R⁴, and z4 are as described herein, including in embodiments.

In embodiments, the compound has the formula:

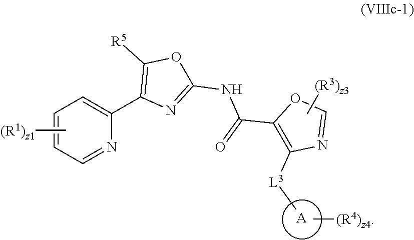

Ring A, L³, R¹, z1, R³, z3, R⁴, z4, and R⁵ are as described herein, including in embodiments.

In embodiments, the compound has the formula:

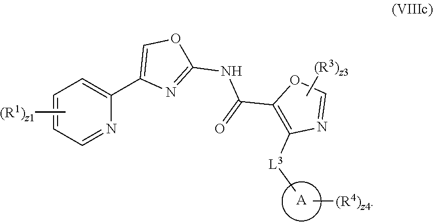

Ring A, L³, R¹, z1, R³, z3, R⁴, and z4 are as described herein, including in embodiments.

In embodiments, the compound has the formula:

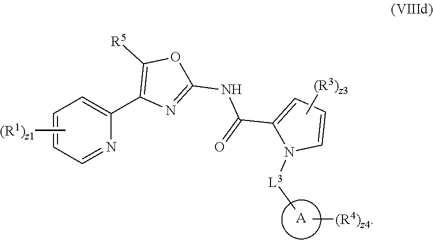

Ring A, L³, R¹, z1, R³, z3, R⁴, z4, and R⁵ are as described herein, including in embodiments.

In embodiments, the compound has the formula:

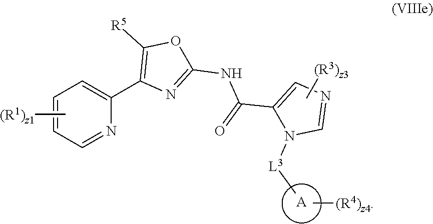

Ring A, L³, R¹, z1, R³, z3, R⁴, z4, and R⁵ are as described herein, including in embodiments.

In embodiments, the compound has the formula:

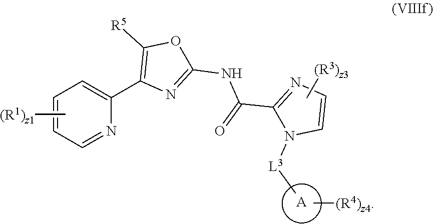

Ring A, L³, R¹, z1, R³, z3, R⁴, z4, and R⁵ are as described herein, including in embodiments.

In embodiments, the compound has the formula:

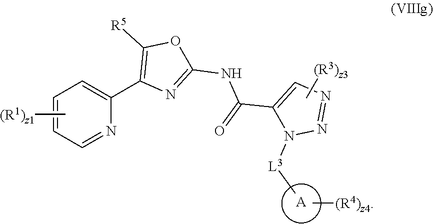

Ring A, L³, R¹, z1, R³, z3, R⁴, z4, and R⁵ are as described herein, including in embodiments.

In embodiments, the compound has the formula:

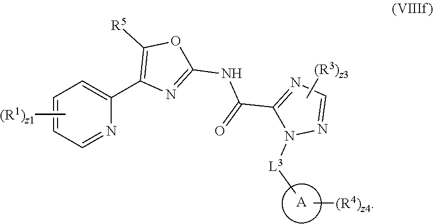

Ring A, L³, R¹, z1, R³, z3, R⁴, z4, and R⁵ are as described herein, including in embodiments.

In embodiments, the compound has the formula:

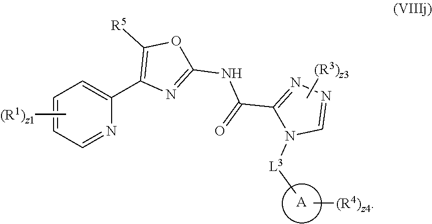

Ring A, L³, R¹, z1, R³, z3, R⁴, z4, and R⁵ are as described herein, including in embodiments.

In embodiments, the compound has the formula:

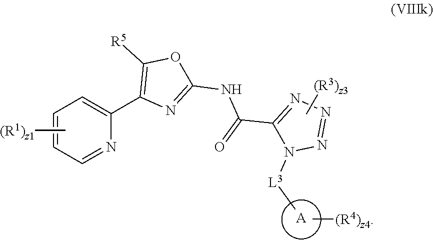

Ring A, L³, R¹, z1, R³, z3, R⁴, z4, and R⁵ are as described herein, including in embodiments.

In embodiments, the compound has the formula:

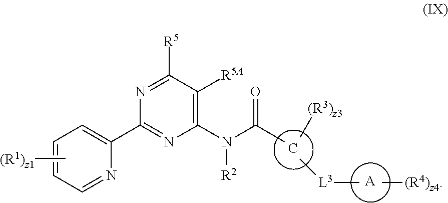

Ring A, Ring C, L³, R¹, z1, R², R³, z3, R⁴, z4, R⁵, and R^{5A} are as described herein, including in embodiments.

In embodiments, the compound has the formula:

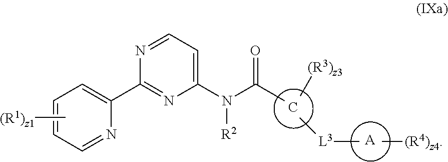

Ring A, Ring C, L³, R¹, z1, R², R³, z3, R⁴, and z4 are as described herein, including in embodiments.

In embodiments, the compound has the formula:

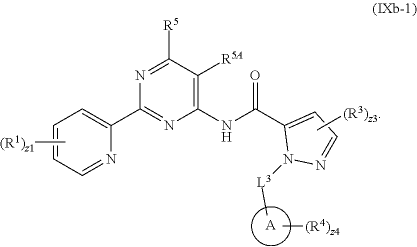

Ring A, L³, R¹, z1, R³, z3, R⁴, z4, R⁵, and R^{5A} are as described herein, including in embodiments.

In embodiments, the compound has the formula:

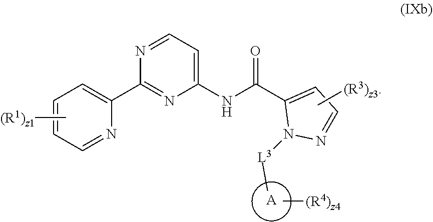

Ring A, L³, R¹, z1, R³, z3, R⁴, and z4 are as described herein, including in embodiments.

In embodiments, the compound has the formula:

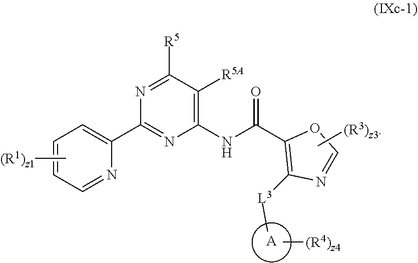

Ring A, L³, R¹, z1, R³, z3, R⁴, z4, R⁵, and R^{5A} are as described herein, including in embodiments.

In embodiments, the compound has the formula:

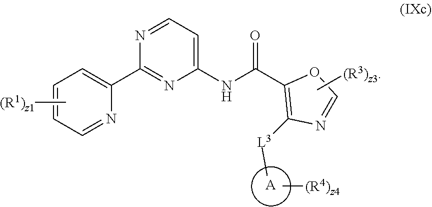

Ring A, L³, R¹, z1, R³, z3, R⁴, and z4 are as described herein, including in embodiments.

In embodiments, the compound has the formula:

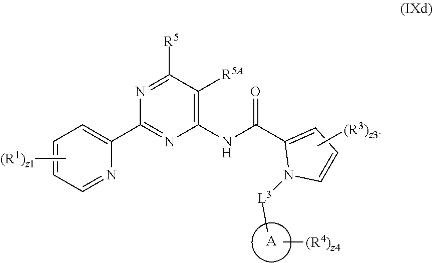

Ring A, L³, R¹, z1, R³, z3, R⁴, z4, R⁵, and R^{5A} are as described herein, including in embodiments.

In embodiments, the compound has the formula:

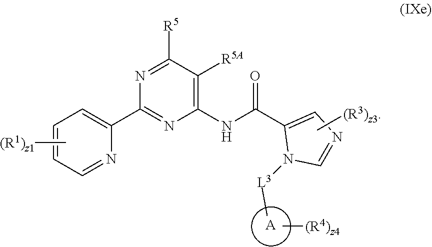

Ring A, L³, R¹, z1, R³, z3, R⁴, z4, R⁵, and R^{5A} are as described herein, including in embodiments.

In embodiments, the compound has the formula:

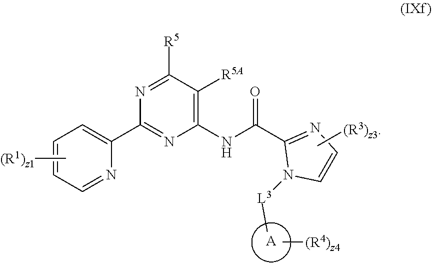

Ring A, L³, R¹, z1, R³, z3, R⁴, z4, R⁵, and R^{5A} are as described herein, including in embodiments.

In embodiments, the compound has the formula:

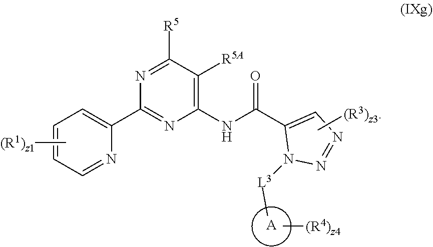

Ring A, L³, R¹, z1, R³, z3, R⁴, z4, R⁵, and R^{5A} are as described herein, including in embodiments.

In embodiments, the compound has the formula:

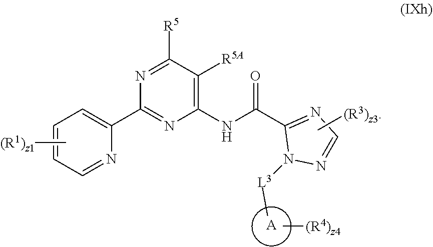

Ring A, L³, R¹, z1, R³, z3, R⁴, z4, R⁵, and R^{5A} are as described herein, including in embodiments.

In embodiments, the compound has the formula:

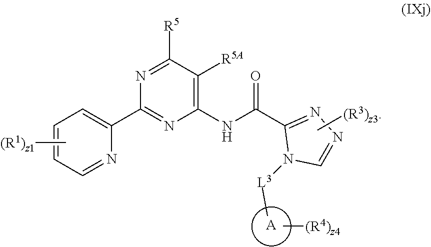

Ring A, L³, R¹, z1, R³, z3, R⁴, z4, R⁵, and R^{5A} are as described herein, including in embodiments.

In embodiments, the compound has the formula:

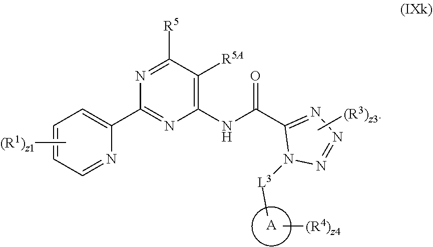

Ring A, L³, R¹, z1, R³, z3, R⁴, z4, R⁵, and R^{5A} are as described herein, including in embodiments.

In an aspect is provided a compound, or a pharmaceutically acceptable salt or tautomer thereof, having the formula:

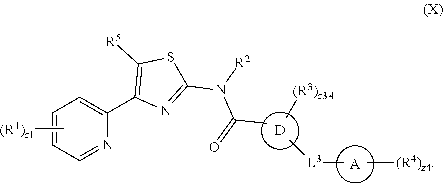

Ring A, L³, R¹, z1, R², R³, R⁴, z4, and R⁵ are as described herein, including in embodiments.

Ring D is pyridonyl, pyrazinyl, pyridyl, or phenyl.

The symbol z3A is an integer from 0 to 4.

In embodiments, the compound has the formula:

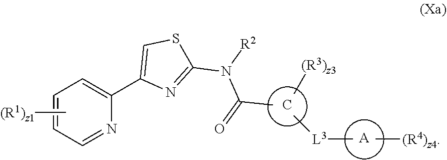

Ring A, Ring D, L³, R¹, z1, R², R³, z3A, R⁴, and z4 are as described herein, including in embodiments.

In embodiments, the compound has the formula:

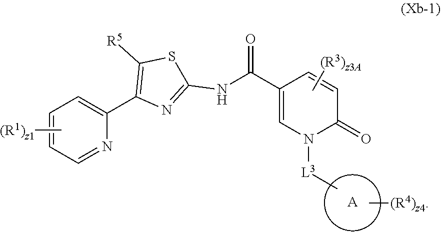

Ring A, L³, R¹, z1, R³, z3A, R⁴, z4, and R⁵ are as described herein, including in embodiments.

In embodiments, the compound has the formula:

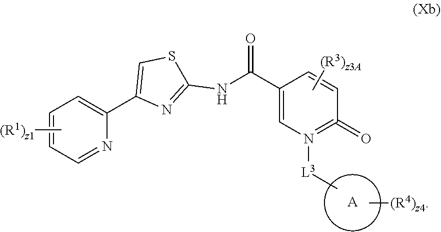

Ring A, L³, R¹, z1, R³, z3A, R⁴, and z4 are as described herein, including in embodiments.

In embodiments, the compound has the formula:

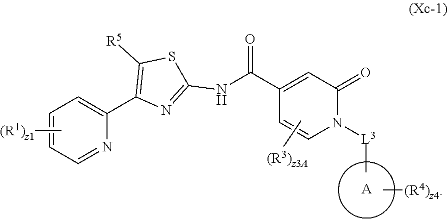

Ring A, L³, R¹, z1, R³, z3A, R⁴, z4, and R⁵ are as described herein, including in embodiments.

In embodiments, the compound has the formula:

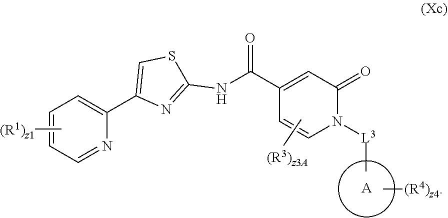

Ring A, L³, R¹, z1, R³, z3A, R⁴, and z4 are as described herein, including in embodiments.

In embodiments, the compound has the formula:

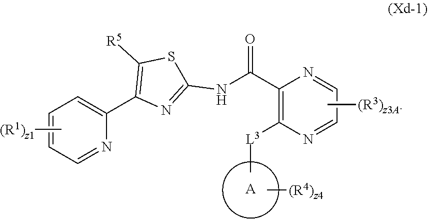

Ring A, L³, R¹, z1, R³, z3A, R⁴, z4, and R⁵ are as described herein, including in embodiments.

In embodiments, the compound has the formula:

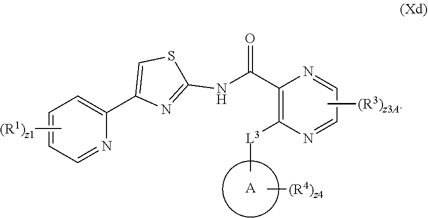

Ring A, L³, R¹, z1, R³, z3A, R⁴, and z4 are as described herein, including in embodiments.

In embodiments, the compound has the formula:

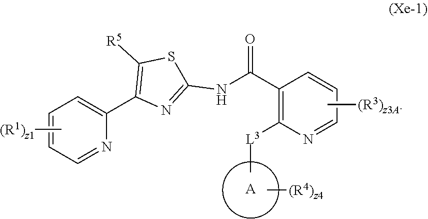

Ring A, L³, R¹, z1, R³, z3A, R⁴, z4, and R⁵ are as described herein, including in embodiments.

In embodiments, the compound has the formula:

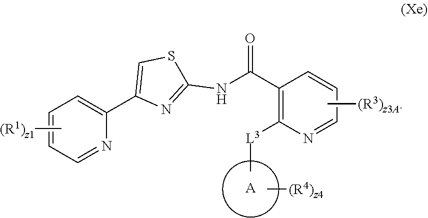

Ring A, L³, R¹, z1, R³, z3A, R⁴, and z4 are as described herein, including in embodiments.

In embodiments, the compound has the formula:

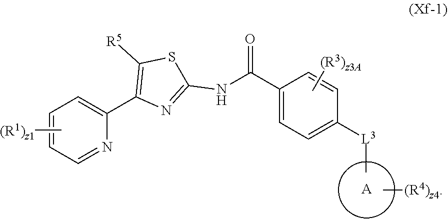

Ring A, L³, R¹, z1, R³, z3A, R⁴, z4, and R⁵ are as described herein, including in embodiments.

In embodiments, the compound has the formula:

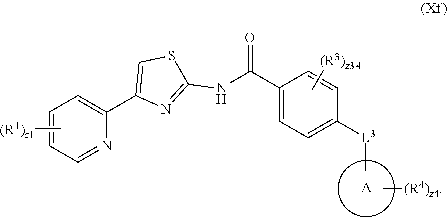

Ring A, L³, R¹, z1, R³, z3A, R⁴, and z4 are as described herein, including in embodiments.

In embodiments, when Ring A is phenyl or 4-pyridyl, then z4 is not 0. In embodiments, when Ring A is phenyl, then at least one R⁴ is not —Cl. In embodiments, when Ring A is phenyl, then at least one R⁴ is not halogen. In embodiments, when Ring A is phenyl and z4 is 1, then R⁴ is not —CH₃. In embodiments, when Ring A is phenyl and z4 is 1, then R⁴ is not unsubstituted C₁-C₄ alkyl.

In embodiments, Ring A is C₃-C₈ cycloalkyl, 3 to 8 membered heterocycloalkyl, phenyl, or 5 to 6 membered heteroaryl. In embodiments, Ring A is C₃-C₈ cycloalkyl. In embodiments, Ring A is 3 to 8 membered heterocycloalkyl. In embodiments, Ring A is phenyl.

In embodiments, Ring A is 5 to 6 membered heteroaryl. In embodiments, Ring A is phenyl, thienyl, or pyridyl. In embodiments, Ring A is phenyl, pyridyl, pyrazinyl, or pyrimidinyl. In embodiments, Ring A is pyridyl. In embodiments, Ring A is 2-pyridyl. In embodiments, Ring A is 3-pyridyl. In embodiments, Ring A is 4-pyridyl. In embodiments, Ring A is pyrazinyl. In embodiments, Ring A is pyrimidinyl. In embodiments, Ring A is 2-pyrimidinyl. In embodiments, Ring A is 4-pyrimidinyl. In embodiments, Ring A is 5-pyrimidinyl. In embodiments, Ring A is pyridazinyl. In embodiments, Ring A is 3-pyridazinyl. In embodiments, Ring A is 4-pyridazinyl. In embodiments, Ring A is thienyl. In embodiments, Ring A is 2-thienyl. In embodiments, Ring A is 3-thienyl. In embodiments, Ring A is thiazolyl. In embodiments, Ring A is 2-thiazolyl. In embodiments, Ring A is 4-thiazolyl. In embodiments, Ring A is 5-thiazolyl. In embodiments, Ring A is isothiazolyl. In embodiments, Ring A is 3-isothiazolyl. In embodiments, Ring A is 4-isothiazolyl. In embodiments, Ring A is 5-isothiazolyl. In embodiments, Ring A is imidazolyl. In embodiments, Ring A is 2-imidazolyl. In embodiments, Ring A is 4-imidazolyl. In embodiments, Ring A is 5-imidazolyl. In embodiments, Ring A is oxazolyl. In embodiments, Ring A is 2-oxazolyl. In embodiments, Ring A is 4-oxazolyl. In embodiments, Ring A is 5-oxazolyl. In embodiments, Ring A is isoxazolyl. In embodiments, Ring A is 3-isoxazolyl. In embodiments, Ring A is 4-isoxazolyl. In embodiments, Ring A is 5-isoxazolyl. In embodiments, Ring A is triazolyl. In embodiments, Ring A is 4-triazolyl. In embodiments, Ring A is 5-triazolyl. In embodiments, Ring A is thiadiazolyl. In embodiments, Ring A is 1,2,3-thiadiazolyl. In embodiments, Ring A is 1,2,5-thiadiazolyl. In embodiments, Ring A is tetrazolyl. In embodiments, Ring A is 5-tetrazolyl.

In embodiments,

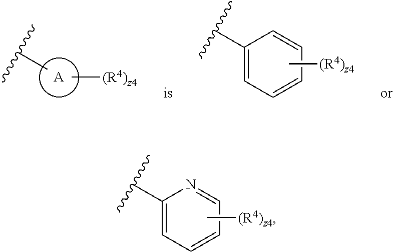

is or wherein R⁴ and z4 are as described herein, including in embodiments. In embodiments,

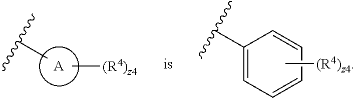

In embodiments,

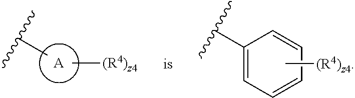

In embodiments

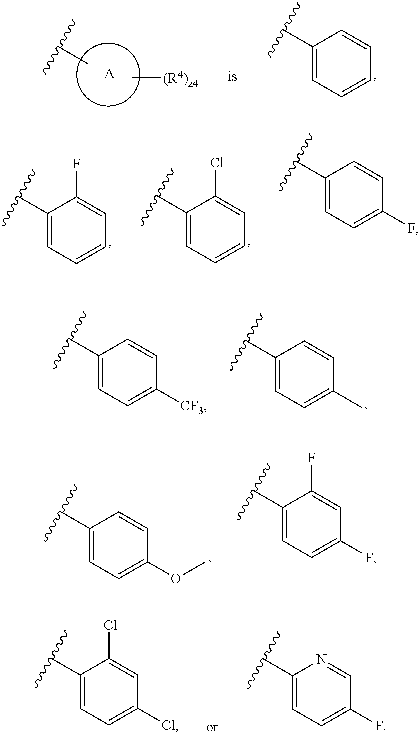

In embodiments,

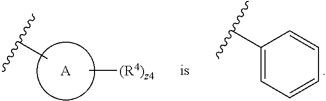

In embodiments,

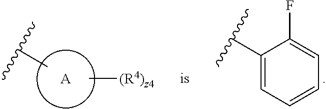

In embodiments,

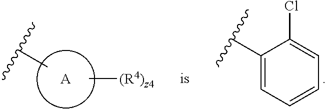

In embodiments,

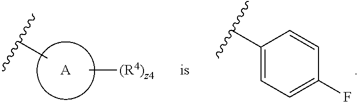

In embodiments,

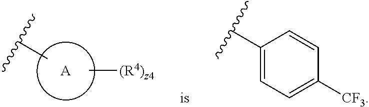

In embodiments,

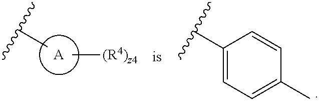

In embodiments,

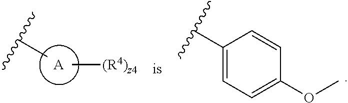

In embodiments,

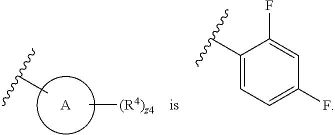

In embodiments,

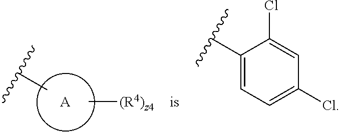

In embodiments,

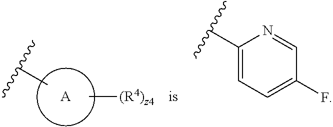

In embodiments,

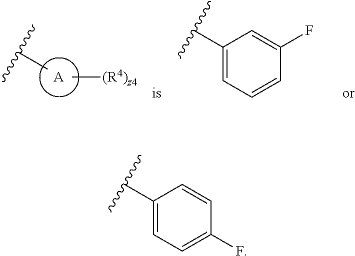

In embodiments,

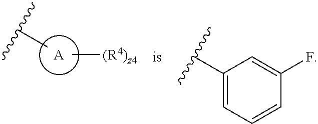

In embodiments,

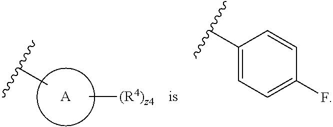

In embodiments,

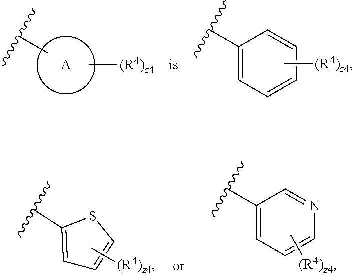

wherein R⁴ and z4 are as described herein, including in embodiments. In embodiments,

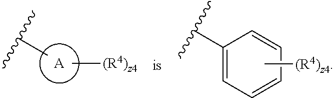

In embodiments,

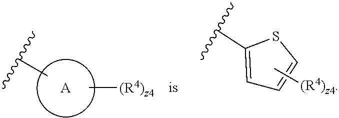

In embodiments,

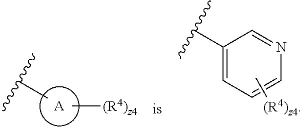

In embodiments,

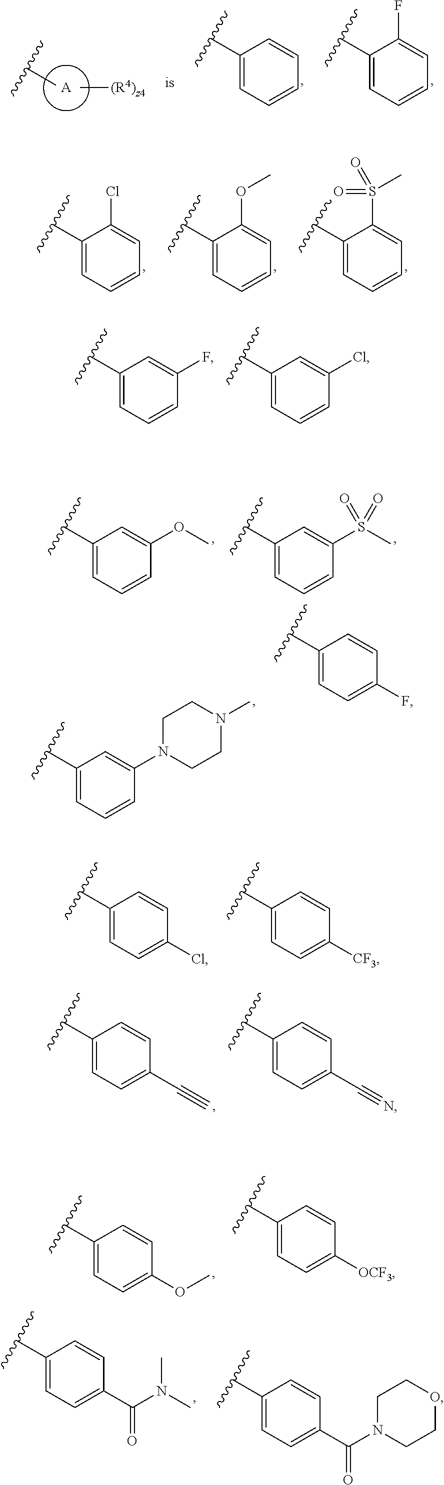

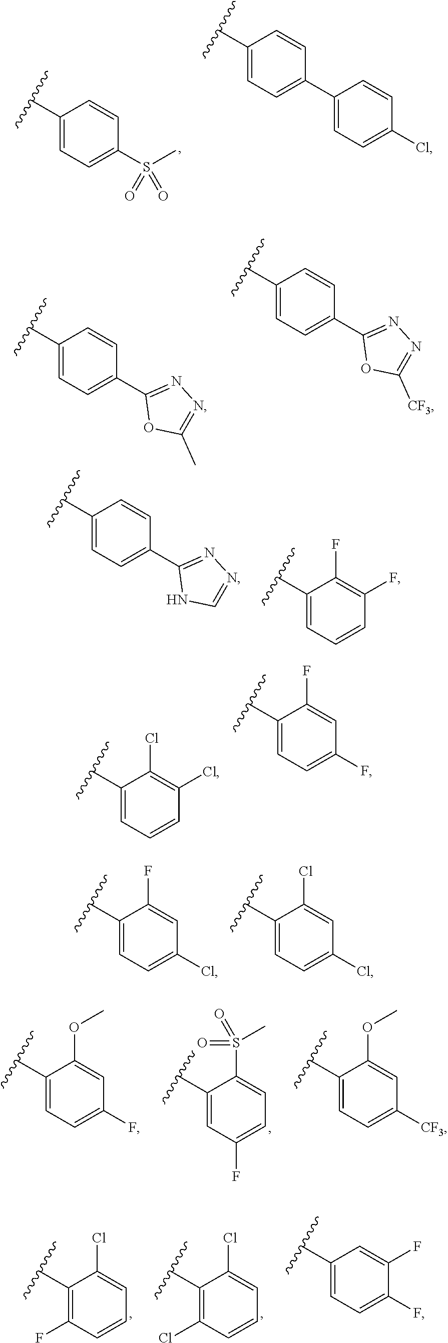

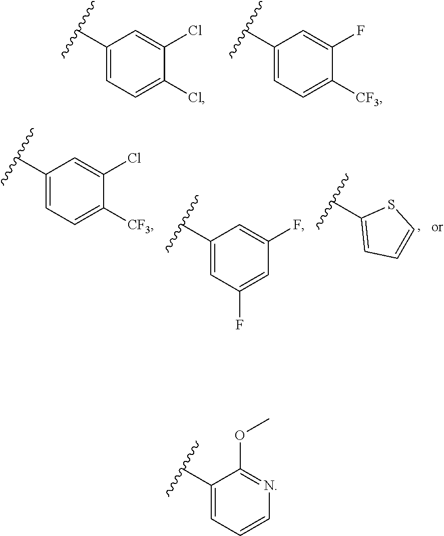

In embodiments,

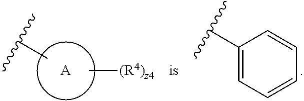

In embodiments,

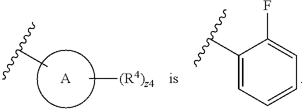

In embodiments,

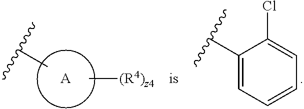

In embodiments,

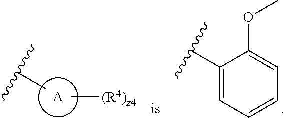

In embodiments,

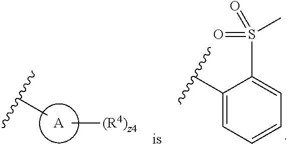

In embodiments

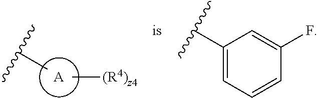

In embodiments,

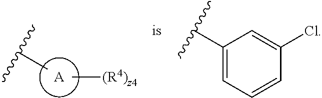

In embodiments

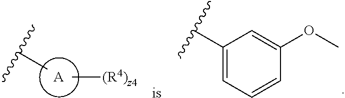

### In

embodiments

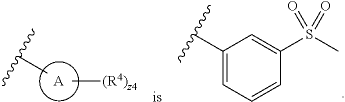

In embodiments,

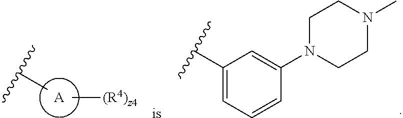

In embodiments,

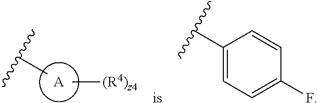

In embodiments,

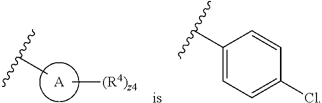

In embodiments

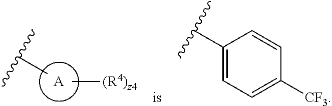

In embodiments

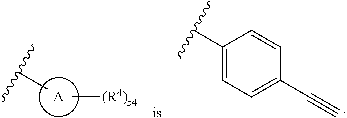

In embodiments,

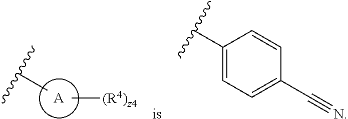

In embodiments

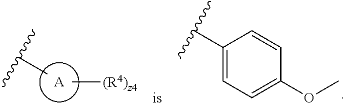

In embodiments

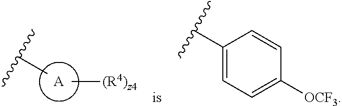

In embodiments,

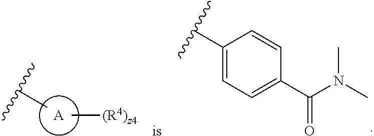

In embodiments,

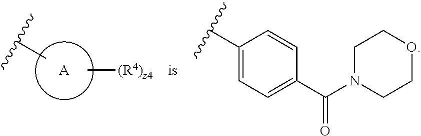

In embodiments

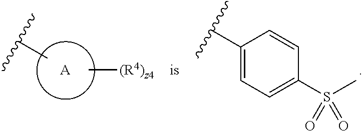

In embodiments,

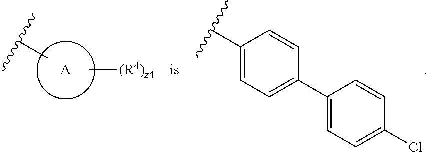

In embodiments,

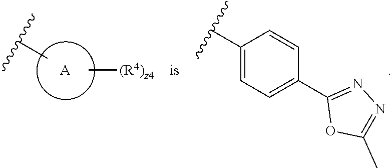

In embodiments,

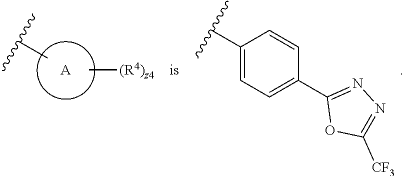

In embodiments,

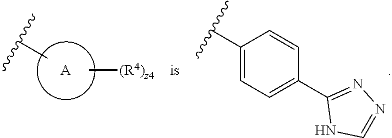

In embodiments,

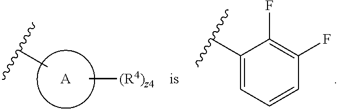

In embodiments,

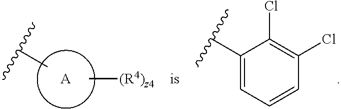

In embodiments,

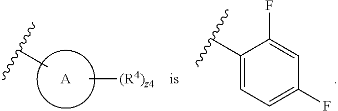

In embodiments,

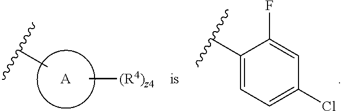

In embodiments,

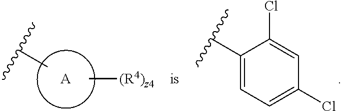

In embodiments,

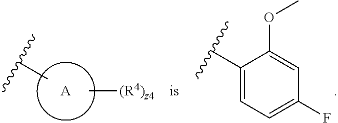

In embodiments,

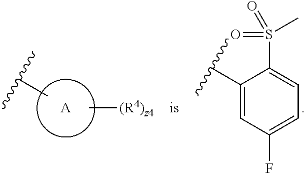

In embodiments

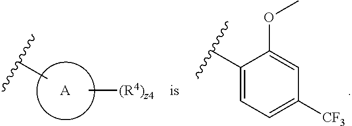

In embodiments,

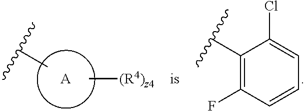

In embodiments,

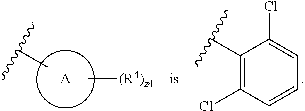

In embodiments,

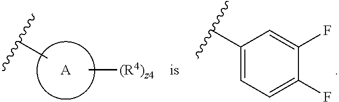

In embodiments,

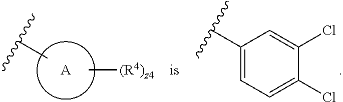

In embodiments,

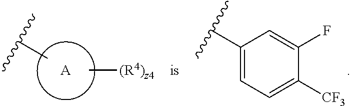

In embodiments,

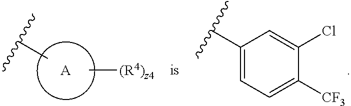

In embodiments

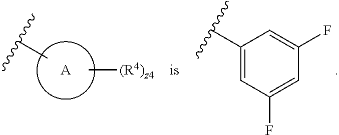

In embodiments,

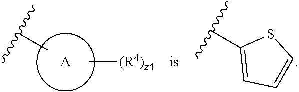

In embodiments

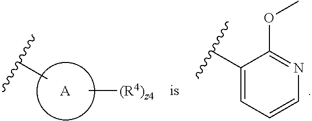

In embodiments

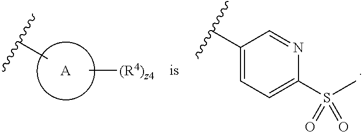

In embodiments

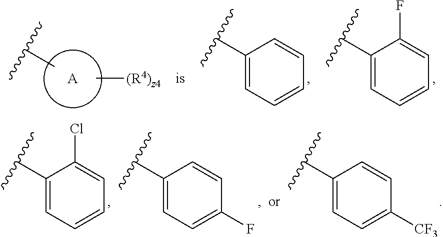

In embodiments

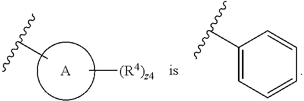

In embodiments

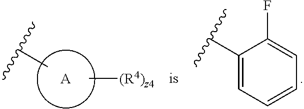

In embodiments

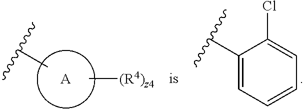

In embodiments,

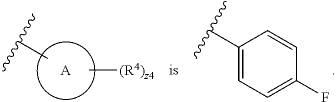

In embodiments,

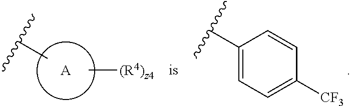

In embodiments, a substituted L¹ (e.g., substituted alkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond or substituted or unsubstituted C₁-C₄ alkylene. In embodiments, L¹ is a bond. In embodiments, L¹ is substituted or unsubstituted C₁-C₄ alkylene.

In embodiments, L¹ is unsubstituted C₁-C₄ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted isopropylene. In embodiments, L¹ is unsubstituted butylene. In embodiments, L¹ is unsubstituted n-butylene. In embodiments, L¹ is unsubstituted isobutylene. In embodiments, L¹ is unsubstituted tert-butylene.

In embodiments, a substituted L² (e.g., substituted alkylene and/or substituted heteroalkylene) is substituted with at least one substituent group, size-limited substituent group, or lower substituent group; wherein if the substituted L² is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, L² is a bond, —C(O)NR¹⁰—, —NR¹⁰C(O)—, —NR¹⁰S(O)₂—, —S(O)₂NR¹⁰—, substituted or unsubstituted C₁-C₄ alkylene, or substituted or unsubstituted 2 to 6 membered heteroalkylene. In embodiments, L² is a bond. In embodiments, L² is —C(O)NR¹⁰—. In embodiments, L² is —C(O)NH—. In embodiments, L² is —NR¹⁰C(O)—. In embodiments, L² is —NHC(O)—. In embodiments, L² is

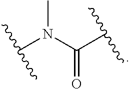 [SMILES: *C(=O)N(*)C]

In embodiments, L² is —NR¹⁰S(O)₂—. In embodiments, L² is —NHS(O)₂—. In embodiments, L² is —S(O)₂NR¹⁰—. In embodiments, L² is —S(O)₂NH—. In embodiments, L² is

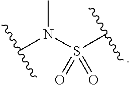 [SMILES: *N(C)S(*)(=O)=O]

In embodiments, L² is substituted or unsubstituted C₁-C₄ alkylene. In embodiments, L² is unsubstituted C₁-C₄ alkylene. In embodiments, L² is unsubstituted methylene. In embodiments, L² is unsubstituted ethylene. In embodiments, L² is unsubstituted propylene. In embodiments, L² is unsubstituted n-propylene. In embodiments, L² is unsubstituted isopropylene. In embodiments, L² is unsubstituted butylene. In embodiments, L² is unsubstituted n-butylene. In embodiments, L² is unsubstituted isobutylene. In embodiments, L² is unsubstituted tert-butylene. In embodiments, L² is substituted or unsubstituted 2 to 6 membered heteroalkylene.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is hydrogen. In embodiments, R¹⁰ is unsubstituted C₁-C₄ alkyl. In embodiments, R¹⁰ is unsubstituted methyl. In embodiments, R¹⁰ is unsubstituted ethyl. In embodiments, R¹⁰ is unsubstituted propyl. In embodiments, R¹⁰ is unsubstituted n-propyl. In embodiments, R¹⁰ is unsubstituted isopropyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl.

In embodiments, a substituted L³ (e.g., substituted alkylene and/or substituted heteroalkylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L³ is a bond, substituted or unsubstituted C₁-C₄ alkylene, or substituted or unsubstituted 2 to 6 membered heteroalkylene. In embodiments, L³ is a bond. In embodiments, L³ is substituted or unsubstituted C₁-C₄ alkylene. In embodiments, L³ is unsubstituted C₁-C₄ alkylene. In embodiments, L³ is unsubstituted methylene. In embodiments, L³ is unsubstituted ethylene. In embodiments, L³ is unsubstituted propylene. In embodiments, L³ is unsubstituted n-propylene. In embodiments, L³ is unsubstituted isopropylene. In embodiments, L³ is unsubstituted butylene. In embodiments, L³ is unsubstituted n-butylene. In embodiments, L³ is unsubstituted isobutylene. In embodiments, L³ is unsubstituted tert-butylene. In embodiments, L³ is substituted or unsubstituted 2 to 6 membered heteroalkylene. In embodiments, L³ is oxo-substituted 2 to 6 membered heteroalkylene. In embodiments, L³ is unsubstituted 2 to 6 membered heteroalkylene.

In embodiments, L³ is a bond, —O—, substituted or unsubstituted C₁-C₄ alkylene, or substituted or unsubstituted 2 to 6 membered heteroalkylene. In embodiments, L³ is a bond, —O—, unsubstituted methylene, or

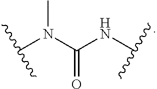 [SMILES: *NC(=O)N(*)C]

In embodiments, L³ is —O—. In embodiments, L³ is

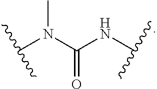 [SMILES: *NC(=O)N(*)C]

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted ring formed when two R¹ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R¹ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R¹ substituents are joined is substituted, it is substituted with at least one substituent group. In embodiments, when the substituted ring formed when two R¹ substituents are joined is substituted, it is substituted with at least one size-limited substituent group. In embodiments, when the substituted ring formed when two R¹ substituents are joined is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R^{1A} is hydrogen. In embodiments, R^{1A} is unsubstituted C₁-C₄ alkyl.

In embodiments, R^{1A} is unsubstituted methyl. In embodiments, R^{1A} is unsubstituted ethyl. In embodiments, R^{1A} is unsubstituted propyl. In embodiments, R^{1A} is unsubstituted n-propyl. In embodiments, R^{1A} is unsubstituted isopropyl. In embodiments, RA is unsubstituted butyl. In embodiments, R^{1A} is unsubstituted n-butyl. In embodiments, R^{1A} is unsubstituted isobutyl. In embodiments, R^{1A} is unsubstituted tert-butyl.

In embodiments, R^{1B} is hydrogen. In embodiments, R^{1B} is unsubstituted C₁-C₄ alkyl.

In embodiments, R^{1B} is unsubstituted methyl. In embodiments, R^{1B} is unsubstituted ethyl. In embodiments, R^{1B} is unsubstituted propyl. In embodiments, R^{1B} is unsubstituted n-propyl. In embodiments, R^{1B} is unsubstituted isopropyl. In embodiments, R^{1B} is unsubstituted butyl. In embodiments, R^{1B} is unsubstituted n-butyl. In embodiments, R^{1B} is unsubstituted isobutyl. In embodiments, R^{1B} is unsubstituted tert-butyl.

In embodiments, R^{1C} is hydrogen. In embodiments, R^{1C} is unsubstituted C₁-C₄ alkyl. In embodiments, R^{1C} is unsubstituted methyl. In embodiments, R^{1C} is unsubstituted ethyl. In embodiments, R^{1C} is unsubstituted propyl. In embodiments, R^{1C} is unsubstituted n-propyl. In embodiments, R^{1C} is unsubstituted isopropyl. In embodiments, R^{1C} is unsubstituted butyl. In embodiments, R^{1C} is unsubstituted n-butyl. In embodiments, R^{1C} is unsubstituted isobutyl. In embodiments, R^{1C} is unsubstituted tert-butyl.

In embodiments, R^{1D} is hydrogen. In embodiments, R^{1D} is unsubstituted C₁-C₄ alkyl. In embodiments, R^{1D} is unsubstituted methyl. In embodiments, R^{1D} is unsubstituted ethyl. In embodiments, R^{1D} is unsubstituted propyl. In embodiments, R^{1D} is unsubstituted n-propyl. In embodiments, R^{1D} is unsubstituted isopropyl. In embodiments, R^{1D} is unsubstituted butyl. In embodiments, R^{1D} is unsubstituted n-butyl. In embodiments, R^{1D} is unsubstituted isobutyl. In embodiments, R^{1D} is unsubstituted tert-butyl.

In embodiments, R¹ is independently halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —CCl₃. In embodiments, R¹ is independently —CBr₃. In embodiments, R¹ is independently —CF₃. In embodiments, R¹ is independently —Cl₃. In embodiments, R¹ is independently —CH₂Cl. In embodiments, R¹ is independently —CH₂Br. In embodiments, R¹ is independently —CH₂F. In embodiments, R¹ is independently —CH₂I. In embodiments, R¹ is independently —CHCl₂. In embodiments, R¹ is independently —CHBr₂. In embodiments, R¹ is independently —CHF₂. In embodiments, R¹ is independently —CHI₂. In embodiments, R¹ is independently —CN. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently —SO₃H. In embodiments, R¹ is independently —OSO₃H. In embodiments, R¹ is independently —SO₂NH₂. In embodiments, R¹ is independently —NHNH₂. In embodiments, R¹ is independently —ONH₂. In embodiments, R¹ is independently —NHC(O)NH₂. In embodiments, R¹ is independently —NHSO₂H. In embodiments, R¹ is independently —NHC(O)H. In embodiments, R′ is independently —NHC(O)OH. In embodiments, R¹ is independently —NHOH. In embodiments, R¹ is independently —OCCl₃. In embodiments, R¹ is independently —OCBr₃. In embodiments, R¹ is independently —OCF₃. In embodiments, R¹ is independently —OCl₃. In embodiments, R¹ is independently —OCH₂Cl. In embodiments, R¹ is independently —OCH₂Br. In embodiments, R¹ is independently —OCH₂F. In embodiments, R¹ is independently —OCH₂I. In embodiments, R¹ is independently —OCHCl₂. In embodiments, R¹ is independently —OCHBr₂. In embodiments, R¹ is independently —OCHF₂. In embodiments, R¹ is independently —OCHI₂. In embodiments, R¹ is independently —SF₅. In embodiments, R¹ is independently —N₃. In embodiments, R¹ is independently unsubstituted C₁-C₄ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted isobutyl. In embodiments, R¹ is independently unsubstituted tert-butyl. In embodiments, R¹ is independently unsubstituted 2 to 6 membered heteroalkyl. In embodiments, R¹ is independently unsubstituted methoxy. In embodiments, R¹ is independently unsubstituted ethoxy. In embodiments, R¹ is independently unsubstituted propoxy. In embodiments, R¹ is independently unsubstituted n-propoxy. In embodiments, R¹ is independently unsubstituted isopropoxy. In embodiments, R¹ is independently unsubstituted butoxy.

In embodiments, R¹ is independently halogen, substituted or unsubstituted C₁-C₄ alkyl, or substituted or unsubstituted 2 to 6 membered heteroalkyl. In embodiments, R¹ is independently —F, unsubstituted methyl,

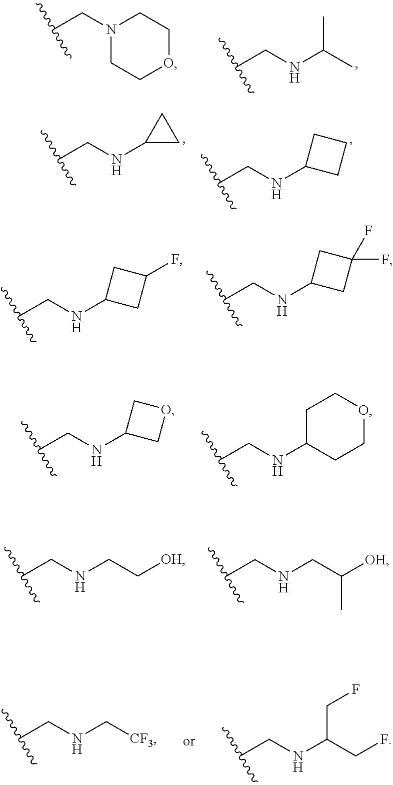

In embodiments, R¹ is independently

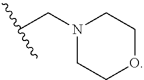 [SMILES: *CN1CCOCC1]

In embodiments, R¹ is independently

In embodiments, R¹ is independently

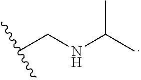 [SMILES: *CNC(C)C]

In embodiments, R¹ is independently

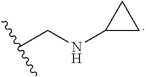 [SMILES: *CNC1CC1]

In embodiments, R¹ is independently

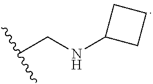 [SMILES: *CNC1CCC1]

In embodiments, R¹ is independently

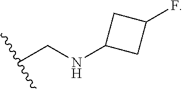 [SMILES: *CNC1CC(F)C1]

In embodiments, R¹ is independently

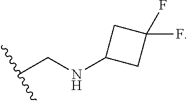 [SMILES: *CNC1CC(F)(F)C1]

In embodiments, R¹ is independently

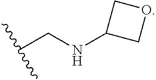 [SMILES: *CNC1COC1]

In embodiments, R¹ is independently

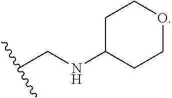 [SMILES: *CNC1CCOCC1]

In embodiments R¹ is independently

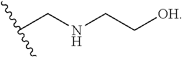 [SMILES: *CNCCO]

In embodiments, R¹ is independently

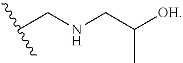 [SMILES: *CNCC(C)O]

In embodiments, R¹ is independently

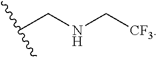 [SMILES: *CNCC(F)(F)F]

In embodiments, R¹ is independently

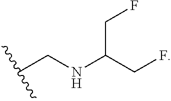 [SMILES: *CNC(CF)CF]

In embodiments, R¹ is independently unsubstituted C₁-C₄ alkynyl. In embodiments, R¹ is independently

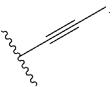 [SMILES: *C#CC]

In embodiments, R¹ is independently

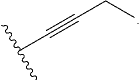 [SMILES: *C#CCC]

In embodiments, z1 is 0. In embodiments, z1 is 1. In embodiments, z1 is 2. In embodiments, z1 is 3. In embodiments, z1 is 4.

In embodiments,

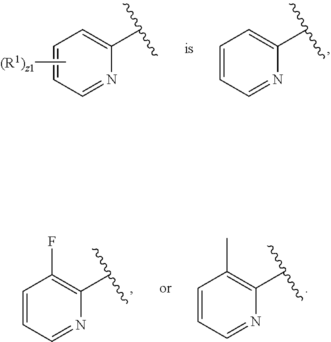

In embodiments,

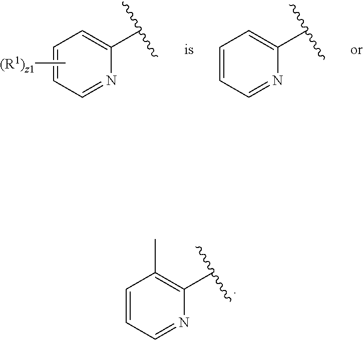

In embodiments,

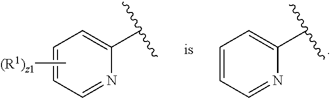

In embodiments,

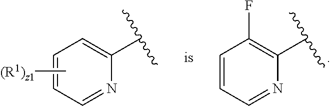

In embodiments,

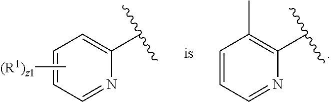

In embodiments,

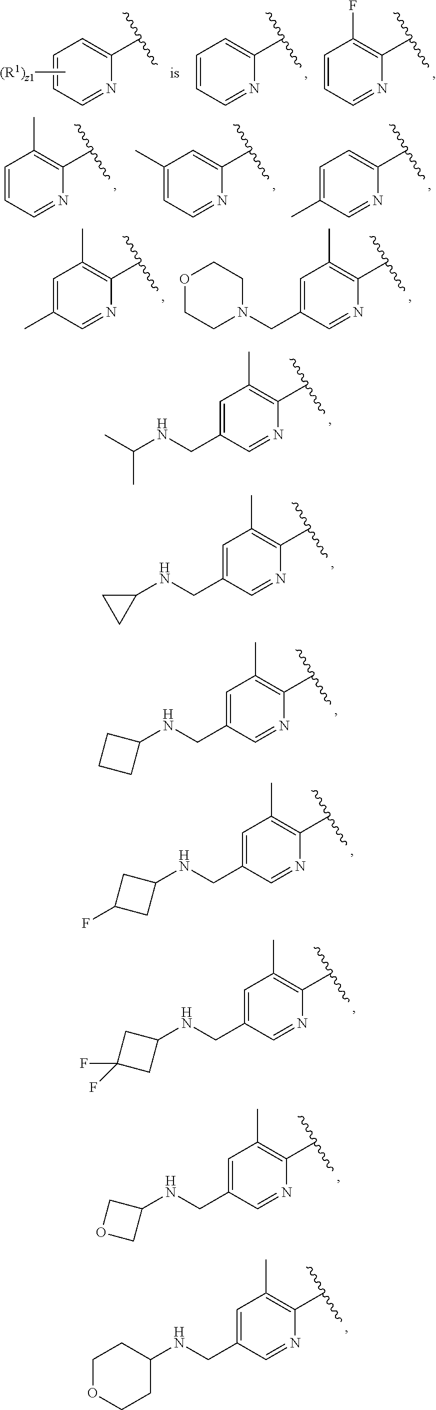

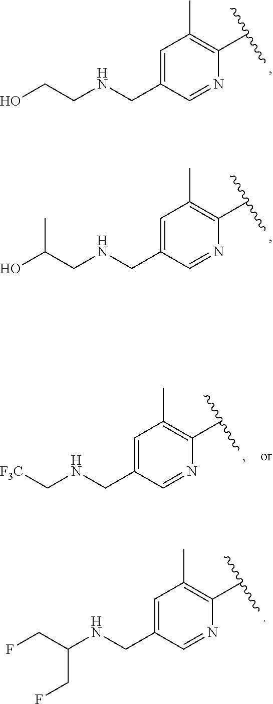 [SMILES: *c1ncc(CNCCO)cc1C ; *c1ncc(CNCC(C)O)cc1C ; *c1ncc(CNCC(F)(F)F)cc1C ; *c1ncc(CNC(CF)CF)cc1C]

In embodiments,

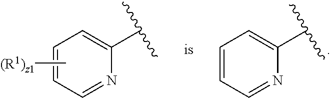

In embodiments,

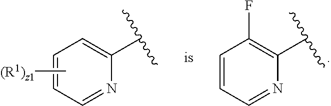

In embodiments,

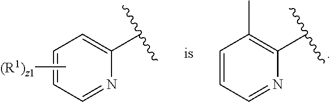

In embodiments,

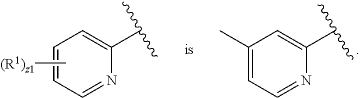

In embodiments, N is

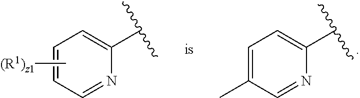

In embodiments,

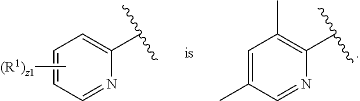

In embodiments,

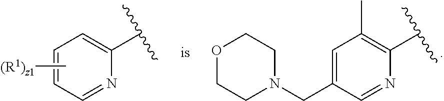

In embodiments,

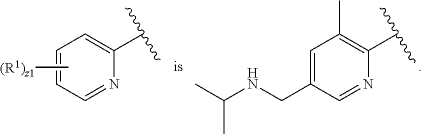

In embodiments,

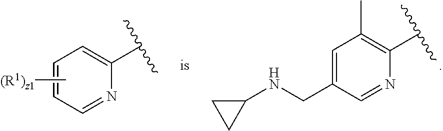

In embodiments,

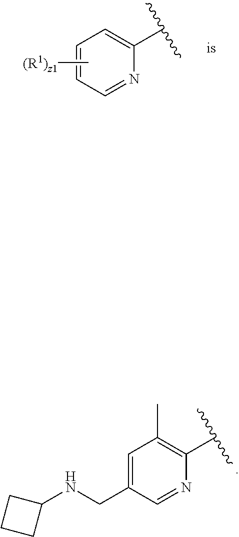

In embodiments,

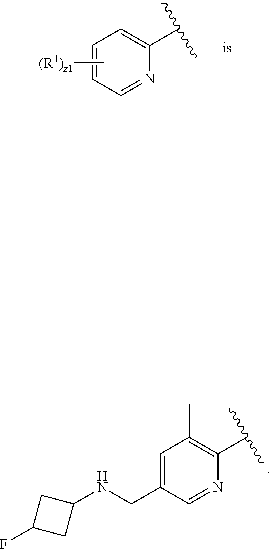

In embodiments,

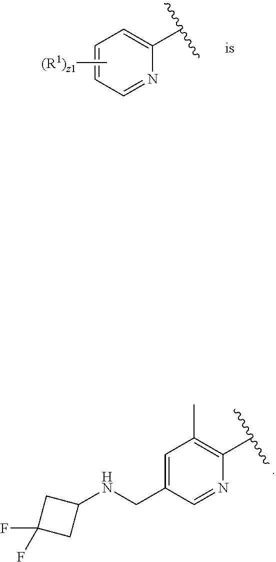

In embodiments,

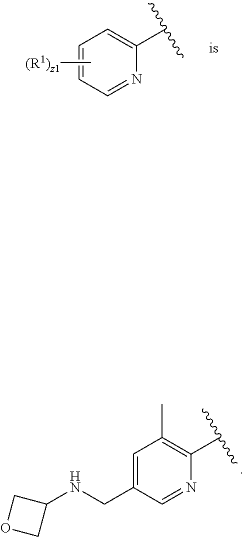

In embodiments,

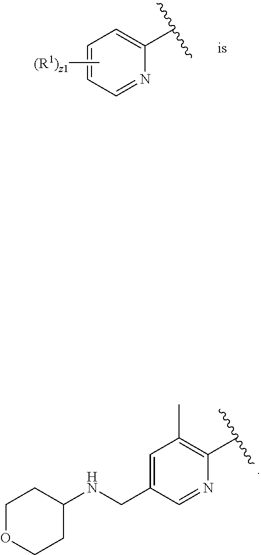

In embodiments,

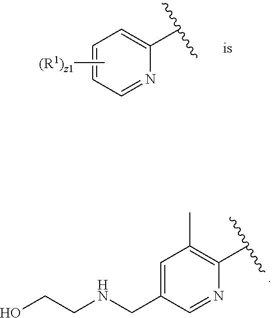

In embodiments,

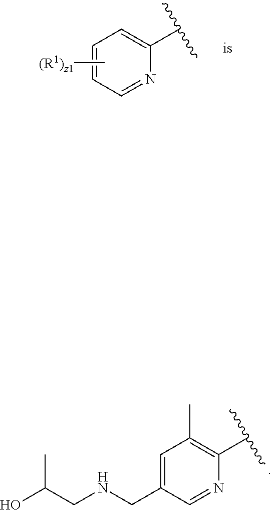

In embodiments,

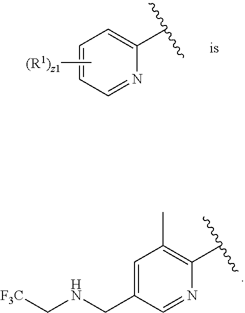

In embodiments,

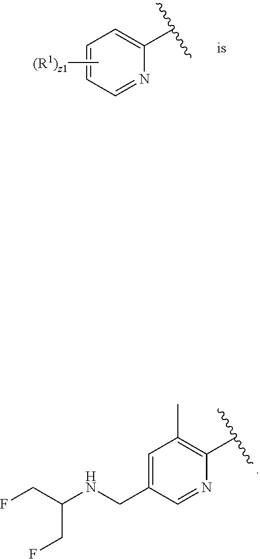

In embodiments, two R¹ substituents are joined to form a substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl. In embodiments, two R¹ substituents are joined to form a substituted or unsubstituted C₃-C₈ cycloalkyl. In embodiments, two R¹ substituents are joined to form a substituted or unsubstituted 3 to 8 membered heterocycloalkyl. In embodiments, two R¹ substituents are joined to form a substituted or unsubstituted phenyl. In embodiments, two R¹ substituents are joined to form an unsubstituted phenyl. In embodiments, two R¹ substituents are joined to form a substituted or unsubstituted 5 to 6 membered heteroaryl. In embodiments, two R¹ substituents are joined to form an unsubstituted pyridyl.

In embodiments, two R¹ substituents are joined to form

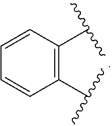 [SMILES: *c1ccccc1*]

In embodiments, two R¹ substituents are joined to form

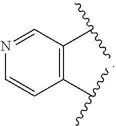 [SMILES: *c1ccncc1*]

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is hydrogen. In embodiments, R² is unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted ring formed when two R³ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R³ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R³ substituents are joined is substituted, it is substituted with at least one substituent group. In embodiments, when the substituted ring formed when two R³ substituents are joined is substituted, it is substituted with at least one size-limited substituent group. In embodiments, when the substituted ring formed when two R³ substituents are joined is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A} and R^{3B}substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R^{3A} is hydrogen. In embodiments, R^{3A} is unsubstituted C₁-C₄ alkyl. In embodiments, R^{3A} is unsubstituted methyl. In embodiments, R^{3A} is unsubstituted ethyl. In embodiments, R^{3A} is unsubstituted propyl. In embodiments, R^{3A} is unsubstituted n-propyl. In embodiments, R^{3A} is unsubstituted isopropyl. In embodiments, R^{3A} is unsubstituted butyl. In embodiments, R^{3A} is unsubstituted n-butyl. In embodiments, R^{3A} is unsubstituted isobutyl. In embodiments, R^{3A} is unsubstituted tert-butyl.

In embodiments, R^{3B} is hydrogen. In embodiments, R^{3B} is unsubstituted C₁-C₄ alkyl. In embodiments, R^{3B} is unsubstituted methyl. In embodiments, R^{3B} is unsubstituted ethyl. In embodiments, R^{3B} is unsubstituted propyl. In embodiments, R^{3B} is unsubstituted n-propyl. In embodiments, R^{3B} is unsubstituted isopropyl. In embodiments, R^{3B} is unsubstituted butyl. In embodiments, R^{3B} is unsubstituted n-butyl. In embodiments, R^{3B} is unsubstituted isobutyl. In embodiments, R^{3B} is unsubstituted tert-butyl.

In embodiments, R^{3C} is hydrogen. In embodiments, R^{3C} is unsubstituted C₁-C₄ alkyl. In embodiments, R^{3C} is unsubstituted methyl. In embodiments, R^{3C} is unsubstituted ethyl. In embodiments, R^{3C} is unsubstituted propyl. In embodiments, R^{3C} is unsubstituted n-propyl. In embodiments, R^{3C} is unsubstituted isopropyl. In embodiments, R^{3C} is unsubstituted butyl. In embodiments, R^{3C} is unsubstituted n-butyl. In embodiments, R^{3C} is unsubstituted isobutyl. In embodiments, R^{3C} is unsubstituted tert-butyl.

In embodiments, R^{3D} is hydrogen. In embodiments, R^{3D} is unsubstituted C₁-C₄ alkyl. In embodiments, R^{3D} is unsubstituted methyl. In embodiments, R^{3D} is unsubstituted ethyl. In embodiments, R^{3D} is unsubstituted propyl. In embodiments, R^{3D} is unsubstituted n-propyl. In embodiments, R^{3D} is unsubstituted isopropyl. In embodiments, R^{3D} is unsubstituted butyl. In embodiments, R^{3D} is unsubstituted n-butyl. In embodiments, R^{3D} is unsubstituted isobutyl. In embodiments, R^{3D} is unsubstituted tert-butyl. In embodiments, R^{3D} is unsubstituted C₃-C₈ cycloalkyl. In embodiments, R^{3D} is unsubstituted cyclopropyl. In embodiments, R^{3D} is unsubstituted cyclobutyl. In embodiments, R^{3D} is unsubstituted cyclopentyl. In embodiments, R^{3D} is unsubstituted cyclohexyl. In embodiments, R^{3D} is unsubstituted cycloheptyl. In embodiments, R^{3D} is unsubstituted cyclooctyl.

In embodiments, R³ is independently halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl.

In embodiments, R³ is independently halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is independently halogen. In embodiments, R³ is independently —F. In embodiments, R³ is independently —Cl. In embodiments, R³ is independently —Br. In embodiments, R³ is independently —I. In embodiments, R³ is independently —CCl₃. In embodiments, R³ is independently —CBr₃. In embodiments, R³ is independently —CF₃. In embodiments, R³ is independently —Cl₃. In embodiments, R³ is independently —CH₂Cl. In embodiments, R³ is independently —CH₂Br. In embodiments, R³ is independently —CH₂F. In embodiments, R³ is independently —CH₂I. In embodiments, R³ is independently —CHCl₂. In embodiments, R³ is independently —CHBr₂. In embodiments, R³ is independently —CHF₂. In embodiments, R³ is independently —CHI₂. In embodiments, R³ is independently —CN. In embodiments, R³ is independently —OH. In embodiments, R³ is independently —NH₂. In embodiments, R³ is independently —COOH. In embodiments, R³ is independently —CONH₂. In embodiments, R³ is independently —NO₂. In embodiments, R³ is independently —SH. In embodiments, R³ is independently —SO₃H. In embodiments, R³ is independently —OSO₃H. In embodiments, R³ is independently —SO₂NH₂. In embodiments, R³ is independently —NHNH₂. In embodiments, R³ is independently —ONH₂. In embodiments, R³ is independently —NHC(O)NH₂. In embodiments, R³ is independently —NHSO₂H. In embodiments, R³ is independently —NHC(O)H. In embodiments, R³ is independently —NHC(O)OH. In embodiments, R³ is independently —NHOH. In embodiments, R³ is independently —OCCl₃. In embodiments, R³ is independently —OCBr₃. In embodiments, R³ is independently —OCF₃. In embodiments, R³ is independently —OCl₃. In embodiments, R³ is independently —OCH₂Cl. In embodiments, R³ is independently —OCH₂Br. In embodiments, R³ is independently —OCH₂F. In embodiments, R³ is independently —OCH₂I. In embodiments, R³ is independently —OCHCl₂. In embodiments, R³ is independently —OCHBr₂. In embodiments, R³ is independently —OCHF₂. In embodiments, R³ is independently —OCHI₂. In embodiments, R³ is independently —SF₅. In embodiments, R³ is independently —N₃. In embodiments, R³ is independently substituted or unsubstituted C₁-C₄ alkyl. In embodiments, R³ is independently unsubstituted C₁-C₄ alkyl. In embodiments, R³ is independently unsubstituted methyl. In embodiments, R³ is independently unsubstituted ethyl. In embodiments, R³ is independently unsubstituted propyl. In embodiments, R³ is independently unsubstituted n-propyl. In embodiments, R³ is independently unsubstituted isopropyl. In embodiments, R³ is independently unsubstituted butyl. In embodiments, R³ is independently unsubstituted n-butyl. In embodiments, R³ is independently unsubstituted isobutyl. In embodiments, R³ is independently unsubstituted tert-butyl. In embodiments, R³ is independently substituted or unsubstituted 2 to 6 membered heteroalkyl. In embodiments, R³ is independently unsubstituted 2 to 6 membered heteroalkyl. In embodiments, R³ is independently unsubstituted methoxy. In embodiments, R³ is independently unsubstituted ethoxy. In embodiments, R³ is independently unsubstituted propoxy. In embodiments, R³ is independently unsubstituted n-propoxy. In embodiments, R³ is independently unsubstituted isopropoxy. In embodiments, R³ is independently unsubstituted butoxy. In embodiments, R³ is independently substituted or unsubstituted C₃-C₅ cycloalkyl. In embodiments, R³ is independently unsubstituted C₃-C₅ cycloalkyl. In embodiments, R³ is independently unsubstituted cyclopropyl. In embodiments, R³ is independently unsubstituted cyclobutyl. In embodiments, R³ is independently unsubstituted cyclopentyl. In embodiments, R³ is independently unsubstituted cyclohexyl. In embodiments, R³ is independently unsubstituted cycloheptyl. In embodiments, R³ is independently unsubstituted cyclooctyl. In embodiments, R³ is independently substituted or unsubstituted phenyl.

In embodiments, R³ is independently halogen, —CX³₃, —CHX³₂, substituted C₁-C₄ alkyl, or substituted or unsubstituted 6 membered heteroaryl. In embodiments, R³ is independently substituted C₁-C₄ alkyl. In embodiments, R³ is independently substituted methyl. In embodiments, R³ is independently substituted ethyl. In embodiments, R³ is independently substituted propyl. In embodiments, R³ is independently substituted n-propyl. In embodiments, R³ is independently substituted isopropyl. In embodiments, R³ is independently substituted butyl. In embodiments, R³ is independently substituted n-butyl. In embodiments, R³ is independently substituted isobutyl. In embodiments, R³ is independently substituted tert-butyl. In embodiments, R³ is independently substituted or unsubstituted 6 membered heteroaryl. In embodiments, R³ is independently substituted or unsubstituted pyridyl. In embodiments, R³ is independently substituted or unsubstituted 2-pyridyl. In embodiments, R³ is independently substituted or unsubstituted 3-pyridyl. In embodiments, R³ is independently substituted or unsubstituted 4-pyridyl.

In embodiments, R³ is independently —Cl, —CF₃, —CHF₂,

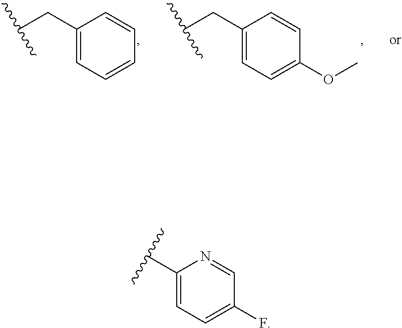

In embodiments, R³ is independently

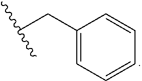 [SMILES: *Cc1ccccc1]

In embodiments, R³ is independently

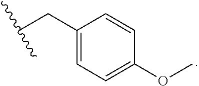 [SMILES: *Cc1ccc(OC)cc1]

In embodiments, R³ is independently

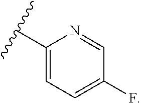 [SMILES: *c1ccc(F)cn1]

In embodiments, R³ is independently substituted or unsubstituted C₁-C₄ alkyl, substituted phenyl, or substituted or unsubstituted 5 to 6 membered heteroaryl. In embodiments, R³ is independently substituted phenyl.

In embodiments, R³ is independently unsubstituted methyl, unsubstituted isopropyl,

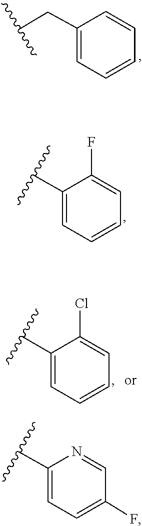 [SMILES: *Cc1ccccc1 ; *c1ccccc1F ; *c1ccccc1Cl ; *c1ccc(F)cn1]

In embodiments, R³ is independently

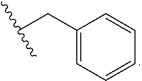 [SMILES: *Cc1ccccc1]

In embodiments, R³ is independently

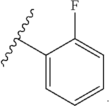 [SMILES: *c1ccccc1F]

In embodiments, R³ is independently

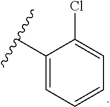 [SMILES: *c1ccccc1Cl]

In embodiments, R³ is independently

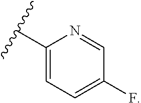 [SMILES: *c1ccc(F)cn1]

In embodiments, R³ is independently —CX³₃, —CHX³₂, —CH₂X³, —NR³ASO₂R^{3D} substituted or unsubstituted C₁-C₄ alkyl, substituted or unsubstituted 2 to 6 membered heteroalkyl, substituted or unsubstituted C₃-C₅ cycloalkyl, or substituted or unsubstituted phenyl. In embodiments, R³ is independently —NR³ASO₂R^{3D}, wherein R^{3A} and R^{3D} are as described herein, including in embodiments.

In embodiments, R³ is independently —CF₃, —CHF₂, unsubstituted methyl, unsubstituted ethyl, unsubstituted isopropyl, unsubstituted tert-butyl, unsubstituted cyclopropyl,

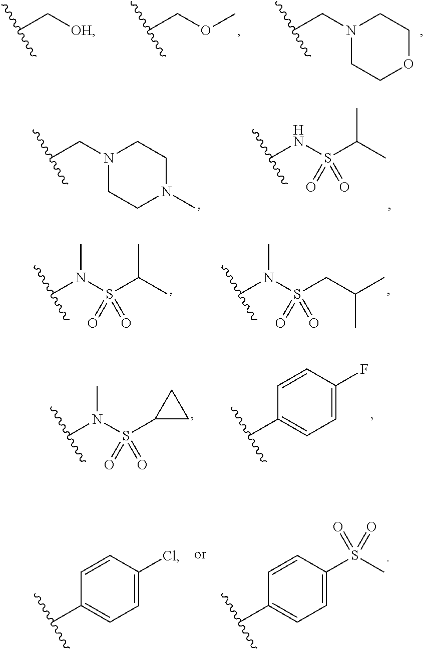

In embodiments, R³ is independently

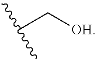 [SMILES: *CO]

In embodiments, R³ is independently

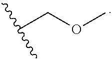 [SMILES: *COC]

In embodiments, R³ is independently

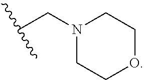 [SMILES: *CN1CCOCC1]

In embodiments, R³ is independently

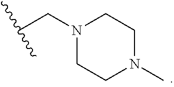 [SMILES: *CN1CCN(C)CC1]

In embodiments, R³ is independently

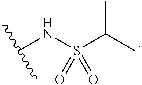 [SMILES: *NS(=O)(=O)C(C)C]

In embodiments, R³ is independently

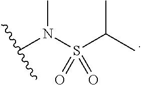 [SMILES: *N(C)S(=O)(=O)C(C)C]

In embodiments, R³ is independently

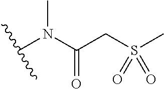 [SMILES: *N(C)C(=O)CS(C)(=O)=O]

In embodiments, R³ is independently

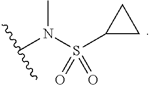 [SMILES: *N(C)S(=O)(=O)C1CC1]

In embodiments, R³ is independently

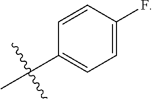 [SMILES: *c1ccc(F)cc1]

In embodiments, R³ is independently

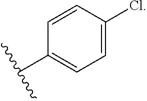 [SMILES: *c1ccc(Cl)cc1]

In embodiments, R³ is independently

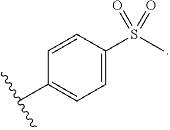 [SMILES: *c1ccc(S(C)(=O)=O)cc1]

In embodiments, R³ is independently halogen, substituted or unsubstituted C₁-C₄ alkyl, or substituted or unsubstituted 2 to 6 membered heteroalkyl.

In embodiments, two R³ substituents are joined to form a substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl. In embodiments, two R³ substituents are joined to form a substituted or unsubstituted C₃-C₈ cycloalkyl. In embodiments, two R³ substituents are joined to form a substituted or unsubstituted 3 to 8 membered heterocycloalkyl. In embodiments, two R³ substituents are joined to form a substituted or unsubstituted phenyl. In embodiments, two R³ substituents are joined to form a substituted or unsubstituted 5 to 6 membered heteroaryl.

In embodiments, two R³ substituents are joined to form

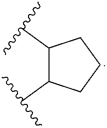 [SMILES: *C1CCCC1*]

In embodiments, z3 is 0. In embodiments, z3 is 1. In embodiments, z3 is 2.

In embodiments, z3A is 0. In embodiments, z3A is 1. In embodiments, z3A is 2. In embodiments, z3A is 3. In embodiments, z3A is 4.

In embodiments, a substituted R^{3.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.1} is substituted with a plurality of groups selected from substituent groups, size-limited substituent groups, and lower substituent groups; each substituent group, size-limited substituent group, and/or lower substituent group may optionally be different. In embodiments, when R^{3.1} is substituted, it is substituted with at least one substituent group. In embodiments, when R^{3.1} is substituted, it is substituted with at least one size-limited substituent group. In embodiments, when R^{3.1} is substituted, it is substituted with at least one lower substituent group.

In embodiments, R^{3.1} is halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R³B —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C} —NR^{3A}C(O)OR^{3C} —NR^{3A}OR^{3C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In embodiments, R^{3.1} is halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{3.1} is halogen. In embodiments, R^{3.1} is —F. In embodiments, R^{3.1} is —Cl. In embodiments, R^{3.1} is —Br. In embodiments, R^{3.1} is —I. In embodiments, R^{3.1} is —CCl₃. In embodiments, R^{3.1} is —CBr₃. In embodiments, R^{3.1} is —CF₃. In embodiments, R^{3.1} is —Cl₃. In embodiments, R^{3.1} is —CH₂Cl. In embodiments, R^{3.1} is —CH₂Br. In embodiments, R^{3.1} is —CH₂F.

In embodiments, R^{3.1} is —CH₂I. In embodiments, R^{3.1} is —CHCl₂. In embodiments, R^{3.1} is —CHBr₂. In embodiments, R^{3.1} is —CHF₂. In embodiments, R^{3.1} is —CHI₂. In embodiments, R^{3.1} is —CN. In embodiments, R^{3.1} is —OH. In embodiments, R^{3.1} is —NH₂. In embodiments, R^{3.1} is —COOH. In embodiments, R^{3.1} is —CONH₂. In embodiments, R^{3.1} is —NO₂. In embodiments, R^{3.1} is —SH. In embodiments, R^{3.1} is —SO₃H. In embodiments, R^{3.1} is —OSO₃H. In embodiments, R^{3.1} is —SO₂NH₂. In embodiments, R^{3.1} is —NHNH₂. In embodiments, R^{3.1} is —ONH₂. In embodiments, R^{3.1} is —NHC(O)NH₂. In embodiments, R^{3.1} is —NHSO₂H. In embodiments, R^{3.1} is —NHC(O)H. In embodiments, R^{3.1} is —NHC(O)OH. In embodiments, R^{3.1} is —NHOH. In embodiments, R^{3.1} is —OCCl₃. In embodiments, R^{3.1} is —OCBr₃. In embodiments, R^{3.1} is —OCF₃.

In embodiments, R^{3.1} is —OCl₃. In embodiments, R^{3.1} is —OCH₂Cl. In embodiments, R^{3.1} is —OCH₂Br. In embodiments, R^{3.1} is —OCH₂F. In embodiments, R^{3.1} is —OCH₂I. In embodiments, R^{3.1} is —OCHCl₂. In embodiments, R^{3.1} is —OCHBr₂. In embodiments, R^{3.1} is —OCHF₂. In embodiments, R^{3.1} is —OCHI₂. In embodiments, R^{3.1} is —SF₅. In embodiments, R^{3.1} is —N₃. In embodiments, R^{3.1} is unsubstituted C₁-C₄ alkyl. In embodiments, R^{3.1} is unsubstituted methyl. In embodiments, R^{3.1} is unsubstituted ethyl. In embodiments, R^{3.1} is unsubstituted propyl. In embodiments, R^{3.1} is unsubstituted n-propyl. In embodiments, R^{3.1} is unsubstituted isopropyl. In embodiments, R^{3.1} is unsubstituted butyl. In embodiments, R^{3.1} is unsubstituted n-butyl. In embodiments, R^{3.1} is unsubstituted isobutyl. In embodiments, R^{3.1} is unsubstituted tert-butyl. In embodiments, R^{3.1} is unsubstituted 2 to 6 membered heteroalkyl. In embodiments, R^{3.1} is unsubstituted methoxy. In embodiments, R^{3.1} is unsubstituted ethoxy. In embodiments, R^{3.1} is unsubstituted propoxy. In embodiments, R^{3.1} is unsubstituted n-propoxy.

In embodiments, R^{3.1} is unsubstituted isopropoxy. In embodiments, R^{3.1} is unsubstituted butoxy.

In embodiments, R^{3.1} is halogen, —CX₃, —CHX³₂, or unsubstituted C₁-C₄ alkyl. In embodiments, R^{3.1} is —Cl, —CF₃, —CHF₂, or unsubstituted methyl. In embodiments, R^{3.1} is —CF₃ or unsubstituted methyl.

In embodiments, a substituted R^{3.2}(e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3.2} is substituted with a plurality of groups selected from substituent groups, size-limited substituent groups, and lower substituent groups; each substituent group, size-limited substituent group, and/or lower substituent group may optionally be different. In embodiments, when R^{3.2} is substituted, it is substituted with at least one substituent group. In embodiments, when R^{3.2} is substituted, it is substituted with at least one size-limited substituent group. In embodiments, when R^{3.2} is substituted, it is substituted with at least one lower substituent group.

In embodiments, R^{3.2} is halogen, —CX₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NR^{3C}C(O)NR^{3A}R³B —N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C} —NR^{3A}C(O)OR^{3C} —NR^{3A}OR^{3C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In embodiments, R^{3.2} is halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{3.2} is halogen. In embodiments, R^{3.2} is —F. In embodiments, R^{3.2} is —Cl. In embodiments, R^{3.2} is —Br. In embodiments, R^{3.2} is —I. In embodiments, R^{3.2} is —CCl₃. In embodiments, R^{3.2} is —CBr₃. In embodiments, R^{3.2} is —CF₃. In embodiments, R^{3.2} is —Cl₃. In embodiments, R^{3.2} is —CH₂Cl. In embodiments, R^{3.2} is —CH₂Br. In embodiments, R^{3.2} is —CH₂F.

In embodiments, R^{3.2} is —CH₂I. In embodiments, R^{3.2} is —CHCl₂. In embodiments, R^{3.2} is —CHBr₂. In embodiments, R^{3.2} is —CHF₂. In embodiments, R^{3.2} is —CHI₂. In embodiments, R^{3.2} is —CN. In embodiments, R^{3.2} is —OH. In embodiments, R^{3.2} is —NH₂. In embodiments, R^{3.2} is —COOH. In embodiments, R^{3.2} is —CONH₂. In embodiments, R^{3.2} is —NO₂. In embodiments, R^{3.2} is —SH. In embodiments, R^{3.2} is —SO₃H. In embodiments, R^{3.2} is —OSO₃H. In embodiments, R^{3.2} is —SO₂NH₂. In embodiments, R^{3.2} is —NHNH₂. In embodiments, R^{3.2} is —ONH₂. In embodiments, R^{3.2} is —NHC(O)NH₂. In embodiments, R^{3.2} is —NHSO₂H. In embodiments, R^{3.2} is —NHC(O)H. In embodiments, R^{3.2} is —NHC(O)OH. In embodiments, R^{3.2} is —NHOH. In embodiments, R^{3.2} is —OCCl₃. In embodiments, R^{3.2} is —OCBr₃. In embodiments, R^{3.2} is —OCF₃.

In embodiments, R^{3.2} is —OCl₃. In embodiments, R^{3.2} is —OCH₂Cl. In embodiments, R^{3.2} is —OCH₂Br. In embodiments, R^{3.2} is —OCH₂F. In embodiments, R^{3.2} is —OCH₂I. In embodiments, R^{3.2} is —OCHCl₂. In embodiments, R^{3.2} is —OCHBr₂. In embodiments, R^{3.2} is —OCHF₂. In embodiments, R^{3.2} is —OCHI₂. In embodiments, R^{3.2} is —SF₅. In embodiments, R^{3.2} is —N₃. In embodiments, R^{3.2} is unsubstituted C₁-C₄ alkyl. In embodiments, R^{3.2} is unsubstituted methyl. In embodiments, R^{3.2} is unsubstituted ethyl. In embodiments, R^{3.2} is unsubstituted propyl. In embodiments, R^{3.2} is unsubstituted n-propyl. In embodiments, R^{3.2} is unsubstituted isopropyl. In embodiments, R^{3.2} is unsubstituted butyl. In embodiments, R^{3.2} is unsubstituted n-butyl. In embodiments, R^{3.2} is unsubstituted isobutyl. In embodiments, R^{3.2} is unsubstituted tert-butyl. In embodiments, R^{3.2} is unsubstituted 2 to 6 membered heteroalkyl. In embodiments, R^{3.2} is unsubstituted methoxy. In embodiments, R^{3.2} is unsubstituted ethoxy. In embodiments, R³₂ is unsubstituted propoxy. In embodiments, R³₂ is unsubstituted n-propoxy.

In embodiments, R^{3.2} is unsubstituted isopropoxy. In embodiments, R^{3.2} is unsubstituted butoxy.

In embodiments, R^{3.2} is halogen, —CX³₃, —CHX³₂, or unsubstituted C₁-C₄ alkyl. In embodiments, R^{3.2} is —Cl, —CF₃, —CHF₂, or unsubstituted methyl. In embodiments, R^{3.2} is —CF₃ or unsubstituted methyl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted ring formed when two R⁴ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R⁴ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R⁴ substituents are joined is substituted, it is substituted with at least one substituent group. In embodiments, when the substituted ring formed when two R⁴ substituents are joined is substituted, it is substituted with at least one size-limited substituent group. In embodiments, when the substituted ring formed when two R⁴ substituents are joined is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴a is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R⁴B is substituted, it is substituted with at least one substituent group. In embodiments, when R⁴B is substituted, it is substituted with at least one size-limited substituent group. In embodiments, when R^{4B} is substituted, it is substituted with at least one lower substituent group.

In embodiments, a substituted ring formed when R^{4A} and R⁴B substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R⁴B substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R⁴B substituents bonded to the same nitrogen atom are joined is substituted, it is substituted with at least one substituent group. In embodiments, when the substituted ring formed when R^{4A} and R^{4B}substituents bonded to the same nitrogen atom are joined is substituted, it is substituted with at least one size-limited substituent group. In embodiments, when the substituted ring formed when R^{4A} and R⁴B substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R^{4A} is hydrogen. In embodiments, R^{4A} is unsubstituted C₁-C₄ alkyl.

In embodiments, R^{4A} is unsubstituted methyl. In embodiments, R^{4A} is unsubstituted ethyl. In embodiments, R^{4A} is unsubstituted propyl. In embodiments, R^{4A} is unsubstituted n-propyl. In embodiments, R^{4A} is unsubstituted isopropyl. In embodiments, R^{4A} is unsubstituted butyl. In embodiments, R^{4A} is unsubstituted n-butyl. In embodiments, R^{4A} is unsubstituted isobutyl. In embodiments, R^{4A} is unsubstituted tert-butyl. In embodiments, R^{4A} is unsubstituted C₃-C₈ cycloalkyl. In embodiments, R^{4A} is unsubstituted cyclopropyl. In embodiments, R^{4A} is unsubstituted cyclobutyl. In embodiments, R^{4A} is unsubstituted cyclopentyl. In embodiments, R^{4A} is unsubstituted cyclohexyl. In embodiments, R^{4A} is unsubstituted cycloheptyl. In embodiments, R^{4A} is unsubstituted cyclooctyl.

In embodiments, R^{4B} is hydrogen. In embodiments, R^{4B} is unsubstituted C₁-C₄ alkyl.

In embodiments, R^{4B} is unsubstituted methyl. In embodiments, R^{4B} is unsubstituted ethyl. In embodiments, R⁴B is unsubstituted propyl. In embodiments, R^{4B} is unsubstituted n-propyl. In embodiments, R⁴B is unsubstituted isopropyl. In embodiments, R^{4B} is unsubstituted butyl. In embodiments, R⁴B is unsubstituted n-butyl. In embodiments, R⁴B is unsubstituted isobutyl. In embodiments, R⁴B is unsubstituted tert-butyl.

In embodiments, R^{4A} and R^{4B} substituents bonded to the same nitrogen atom are joined to form a substituted or unsubstituted 3 to 8 membered heterocycloalkyl. In embodiments, R^{4A} and R^{4B} substituents bonded to the same nitrogen atom are joined to form a substituted or unsubstituted morpholinyl. In embodiments, R^{4A} and R⁴B substituents bonded to the same nitrogen atom are joined to form an unsubstituted morpholinyl. In embodiments, R^{4A} and R^{4B}substituents bonded to the same nitrogen atom are joined to form a substituted or unsubstituted piperazinyl.

In embodiments, R^{4C} is hydrogen. In embodiments, R^{4C} is unsubstituted C₁-C₄ alkyl.

In embodiments, R^{4C} is unsubstituted methyl. In embodiments, R^{4C} is unsubstituted ethyl. In embodiments, R^{4C} is unsubstituted propyl. In embodiments, R^{4C} is unsubstituted n-propyl. In embodiments, R^{4C} is unsubstituted isopropyl. In embodiments, R^{4C} is unsubstituted butyl. In embodiments, R^{4C} is unsubstituted n-butyl. In embodiments, R^{4C} is unsubstituted isobutyl. In embodiments, R^{4C} is unsubstituted tert-butyl.

In embodiments, R^{4D} is hydrogen. In embodiments, R^{4D} is unsubstituted C₁-C₄ alkyl.

In embodiments, R^{4D} is unsubstituted methyl. In embodiments, R^{4D} is unsubstituted ethyl. In embodiments, R^{4D} is unsubstituted propyl. In embodiments, R^{4D} is unsubstituted n-propyl. In embodiments, R^{4D} is unsubstituted isopropyl. In embodiments, R^{4D} is unsubstituted butyl. In embodiments, R^{4D} is unsubstituted n-butyl. In embodiments, R^{4D} is unsubstituted isobutyl. In embodiments, R^{4D} is unsubstituted tert-butyl.

In embodiments, R⁴ is independently oxo, halogen, —CCl₃, —CBr₃, —CF₃, —Cl₃, —CH₂Cl, —CH₂Br, —CH₂F, —CH₂I, —CHC₂, —CHBr₂, —CHF₂, —CHI₂, —CN, —OH, —NH₂, —COGH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl.

In embodiments, R⁴ is independently halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl.

In embodiments, R⁴ is independently oxo. In embodiments, R⁴ is independently halogen. In embodiments, R⁴ is independently —F. In embodiments, R⁴ is independently —Cl. In embodiments, R⁴ is independently —Br. In embodiments, R⁴ is independently —I. In embodiments, R⁴ is independently —CCl₃. In embodiments, R⁴ is independently —CBr₃. In embodiments, R⁴ is independently —CF₃. In embodiments, R⁴ is independently —Cl₃. In embodiments, R⁴ is independently —CH₂Cl. In embodiments, R⁴ is independently —CH₂Br. In embodiments, R⁴ is independently —CH₂F. In embodiments, R⁴ is independently —CH₂I. In embodiments, R⁴ is independently —CHCl₂. In embodiments, R⁴ is independently —CHBr₂. In embodiments, R⁴ is independently —CHF₂. In embodiments, R⁴ is independently —CHI₂. In embodiments, R⁴ is independently —CN. In embodiments, R⁴ is independently —OH. In embodiments, R⁴ is independently —NH₂. In embodiments, R⁴ is independently —COOH. In embodiments, R⁴ is independently —CONH₂. In embodiments, R⁴ is independently —NO₂. In embodiments, R⁴ is independently —SH. In embodiments, R⁴ is independently —SO₃H. In embodiments, R⁴ is independently —OSO₃H. In embodiments, R⁴ is independently —SO₂NH₂. In embodiments, R⁴ is independently —NHNH₂. In embodiments, R⁴ is independently —ONH₂. In embodiments, R⁴ is independently —NHC(O)NH₂. In embodiments, R⁴ is independently —NHSO₂H. In embodiments, R⁴ is independently —NHC(O)H. In embodiments, R⁴ is independently —NHC(O)OH. In embodiments, R⁴ is independently —NHOH. In embodiments, R⁴ is independently —OCCl₃. In embodiments, R⁴ is independently —OCBr₃. In embodiments, R⁴ is independently —OCF₃. In embodiments, R⁴ is independently —OCl₃. In embodiments, R⁴ is independently —OCH₂Cl. In embodiments, R⁴ is independently —OCH₂Br. In embodiments, R⁴ is independently —OCH₂F. In embodiments, R⁴ is independently —OCH₂I. In embodiments, R⁴ is independently —OCHCl₂. In embodiments, R⁴ is independently —OCHBr₂. In embodiments, R⁴ is independently —OCHF₂. In embodiments, R⁴ is independently —OCHI₂. In embodiments, R⁴ is independently —SF₅. In embodiments, R⁴ is independently —N₃. In embodiments, R⁴ is independently substituted or unsubstituted C₁-C₄ alkyl. In embodiments, R⁴ is independently unsubstituted C₁-C₄ alkyl. In embodiments, R⁴ is independently unsubstituted methyl. In embodiments, R⁴ is independently unsubstituted ethyl. In embodiments, R⁴ is independently unsubstituted propyl. In embodiments, R⁴ is independently unsubstituted n-propyl. In embodiments, R⁴ is independently unsubstituted isopropyl. In embodiments, R⁴ is independently unsubstituted butyl. In embodiments, R⁴ is independently unsubstituted n-butyl. In embodiments, R⁴ is independently unsubstituted isobutyl. In embodiments, R⁴ is independently unsubstituted tert-butyl. In embodiments, R⁴ is independently substituted or unsubstituted 2 to 6 membered heteroalkyl. In embodiments, R⁴ is independently unsubstituted 2 to 6 membered heteroalkyl. In embodiments, R⁴ is independently unsubstituted methoxy. In embodiments, R⁴ is independently unsubstituted ethoxy. In embodiments, R⁴ is independently unsubstituted propoxy. In embodiments, R⁴ is independently unsubstituted n-propoxy. In embodiments, R⁴ is independently unsubstituted isopropoxy. In embodiments, R⁴ is independently unsubstituted butoxy. In embodiments, R⁴ is independently substituted or unsubstituted phenyl. In embodiments, R⁴ is independently substituted or unsubstituted 5 to 6 membered heteroaryl. In embodiments, R⁴ is independently substituted or unsubstituted oxadiazolyl. In embodiments, R⁴ is independently substituted or unsubstituted triazolyl. In embodiments, R⁴ is independently unsubstituted triazolyl.

In embodiments, R⁴ is independently halogen, —CX⁴₃, —OCX⁴₃, —SOₙ₄R^{4D}, —SOᵥ₄NR^{4A}R^{4B}, —C(O)OR^{4C}, —C(O)NR^{4A}R^{4B} substituted or unsubstituted C₁-C₄ alkyl, or substituted or unsubstituted 2 to 6 membered heteroalkyl. In embodiments, R⁴ is independently —SOₙ₄R^{4D}, wherein n4 and R^{4D} are as described herein, including in embodiments. In embodiments, R⁴ is independently —SOᵥ₄NR^{4A}R^{4B}, wherein v4, R^{4A}, and R^{4B} are as described herein, including in embodiments. In embodiments, R⁴ is independently —C(O)OR^{4C}, wherein R^{4C} are as described herein, including in embodiments. In embodiments, R⁴ is independently —C(O)NR^{4A}R^{4B}, wherein R^{4A} and R^{4B} are as described herein, including in embodiments.

In embodiments, R⁴ is independently —F, —Cl, —CF₃, —OCF₃, unsubstituted methyl, or unsubstituted methoxy.

In embodiments, R⁴ is independently halogen, —CX⁴₃, —OCX⁴₃, —CN, —SOₙ₄R^{4D}, —C(O)NR^{4A}R^{4B}, —OR^{4D}, substituted or unsubstituted C₁-C₄ alkyl, substituted or unsubstituted 2 to 6 membered heteroalkyl, substituted or unsubstituted 3 to 8 membered heterocycloalkyl, substituted or unsubstituted phenyl, or substituted or unsubstituted 5 to 6 membered heteroaryl.

In embodiments, R⁴ is independently —SOₙ₄R^{4D}, wherein n4 and R^{4D} are as described herein, including in embodiments. In embodiments, R⁴ is independently —C(O)NR^{4A}R^{4B}, wherein R^{4A} and R^{4B} are as described herein, including in embodiments. In embodiments, R⁴ is independently —OR^{4D}, wherein R^{4D} is as described herein, including in embodiments.

In embodiments, R⁴ is independently —F, —Cl, —CF₃, —OCF₃, —OCH₃, —CN,

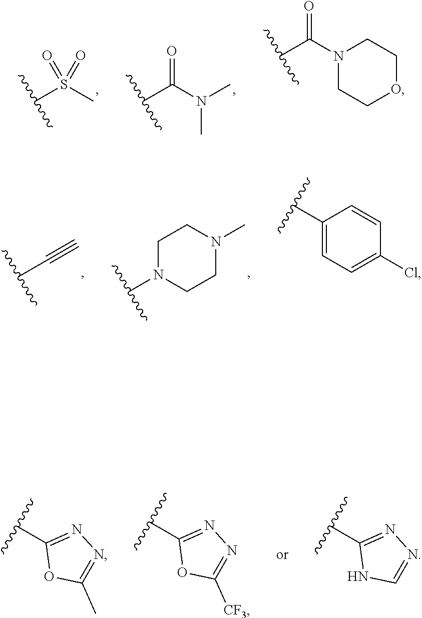

In embodiments, R⁴ is independently

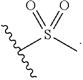 [SMILES: *S(C)(=O)=O]

In embodiments, R⁴ is independently

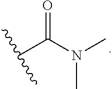 [SMILES: *C(=O)N(C)C]

In embodiments, R⁴ is independently

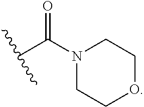 [SMILES: *C(=O)N1CCOCC1]

In embodiments, R⁴ is independently

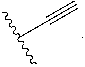 [SMILES: *C#C]

In embodiments, R⁴ is independently

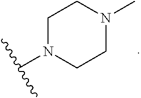 [SMILES: *N1CCN(C)CC1]

In embodiments, R⁴ is independently

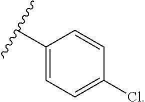 [SMILES: *c1ccc(Cl)cc1]

In embodiments, R⁴ is independently

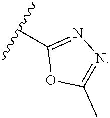 [SMILES: *c1nnc(C)o1]

In embodiments, R⁴ is independently

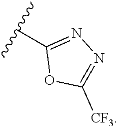 [SMILES: *c1nnc(C(F)(F)F)o1]

In embodiments, R⁴ is independently

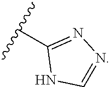 [SMILES: *c1nnc[nH]1]

In embodiments, z4 is 0. In embodiments, z4 is 1. In embodiments, z4 is 2. In embodiments, z4 is 3. In embodiments, z4 is 4. In embodiments, z4 is 5. In embodiments, z4 is 6. In embodiments, z4 is 7. In embodiments, z4 is 8. In embodiments, z4 is 9. In embodiments, z4 is 10. In embodiments, z4 is 11.

In embodiments, a substituted R^{4.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4.1} is substituted with a plurality of groups selected from substituent groups, size-limited substituent groups, and lower substituent groups; each substituent group, size-limited substituent group, and/or lower substituent group may optionally be different. In embodiments, when R^{4.1} is substituted, it is substituted with at least one substituent group. In embodiments, when R^{4.1} is substituted, it is substituted with at least one size-limited substituent group. In embodiments, when R^{4.1} is substituted, it is substituted with at least one lower substituent group.

In embodiments, R^{4.1} is halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D},, —NR^{4A}SO₂R^{4D}, —NR^{4A}C(O)R^{4C} —NR^{4A}C(O)OR^{4C}, —NR^{4A}OR^{4C}, —SF₅, —N₃, substituted or unsubstituted alkyl (e.g., C₁-C₅, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In embodiments, R^{4.1} is halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{4.1} is halogen. In embodiments, R^{4.1} is —F. In embodiments, R^{4.1} is —Cl. In embodiments, R^{4.1} is —Br. In embodiments, R^{4.1} is —I. In embodiments, R^{4.1} is —CCl₃. In embodiments, R^{4.1} is —CBr₃. In embodiments, R^{4.1} is —CF₃. In embodiments, R^{4.1} is —Cl₃. In embodiments, R^{4.1} is —CH₂Cl. In embodiments, R^{4.1} is —CH₂Br. In embodiments, R^{4.1} is —CH₂F. In embodiments, R^{4.1} is —CH₂I. In embodiments, R^{4.1} is —CHCl₂. In embodiments, R^{4.1} is —CHBr₂. In embodiments, R^{4.1} is —CHF₂. In embodiments, R^{4.1} is —CHI₂. In embodiments, R^{4.1} is —CN. In embodiments, R^{4.1} is —OH. In embodiments, R^{4.1} is —NH₂. In embodiments, R^{4.1} is —COOH. In embodiments, R^{4.1} is —CONH₂. In embodiments, R^{4.1} is —NO₂. In embodiments, R^{4.1} is —SH. In embodiments, R^{4.1} is —SO₃H. In embodiments, R^{4.1} is —OSO₃H. In embodiments, R^{4.1} is —SO₂NH₂. In embodiments, R^{4.1} is —NHNH₂. In embodiments, R^{4.1} is —ONH₂. In embodiments, R^{4.1} is —NHC(O)NH₂. In embodiments, R^{4.1} is —NHSO₂H. In embodiments, R^{4.1} is —NHC(O)H. In embodiments, R^{4.1} is —NHC(O)OH. In embodiments, R^{4.1} is —NHOH. In embodiments, R^{4.1} is —OCCl₃. In embodiments, R^{4.1} is —OCBr₃. In embodiments, R^{4.1} is —OCF₃.

In embodiments, R^{4.1} is —OCl₃. In embodiments, R^{4.1} is —OCH₂Cl. In embodiments, R^{4.1} is —OCH₂Br. In embodiments, R^{4.1} is —OCH₂F. In embodiments, R^{4.1} is —OCH₂I. In embodiments, R^{4.1} is —OCHCl₂. In embodiments, R^{4.1} is —OCHBr₂. In embodiments, R^{4.1} is —OCHF₂. In embodiments, R^{4.1} is —OCHI₂. In embodiments, R^{4.1} is —SF₅. In embodiments, R^{4.1} is —N₃. In embodiments, R^{4.1} is unsubstituted C₁-C₄ alkyl. In embodiments, R^{4.1} is unsubstituted methyl. In embodiments, R^{4.1} is unsubstituted ethyl. In embodiments, R^{4.1} is unsubstituted propyl. In embodiments, R^{4.1} is unsubstituted n-propyl. In embodiments, R^{4.1} is unsubstituted isopropyl. In embodiments, R^{4.1} is unsubstituted butyl. In embodiments, R^{4.1} is unsubstituted n-butyl. In embodiments, R^{4.1} is unsubstituted isobutyl. In embodiments, R^{4.1} is unsubstituted tert-butyl. In embodiments, R^{4.1} is unsubstituted 2 to 6 membered heteroalkyl. In embodiments, R^{4.1} is unsubstituted methoxy. In embodiments, R^{4.1} is unsubstituted ethoxy. In embodiments, R^{4.1} is unsubstituted propoxy. In embodiments, R^{4.1} is unsubstituted n-propoxy.

In embodiments, R^{4.1} is unsubstituted isopropoxy. In embodiments, R^{4.1} is unsubstituted butoxy.

In embodiments, R^{4.1} is halogen, —CX⁴₃, or unsubstituted C₁-C₄ alkyl. In embodiments, R^{4.1} is —F, —Cl, —CF₃, or unsubstituted methyl. In embodiments, R^{4.1} is —F or —Cl.

In embodiments, a substituted R⁴₃ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴₃ is substituted with a plurality of groups selected from substituent groups, size-limited substituent groups, and lower substituent groups; each substituent group, size-limited substituent group, and/or lower substituent group may optionally be different. In embodiments, when R^{4.3} is substituted, it is substituted with at least one substituent group. In embodiments, when R^{4.3} is substituted, it is substituted with at least one size-limited substituent group. In embodiments, when R^{4.3} is substituted, it is substituted with at least one lower substituent group.

In embodiments, R^{4.3} is halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D},, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In embodiments, R^{4.3} is halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{4.3} is halogen. In embodiments, R^{4.3} is —F. In embodiments, R^{4.3} is —Cl. In embodiments, R^{4.3} is —Br. In embodiments, R^{4.3} is —I. In embodiments, R^{4.3} is —CCl₃. In embodiments, R^{4.3} is —CBr₃. In embodiments, R^{4.3} is —CF₃. In embodiments, R^{4.3} is —Cl₃. In embodiments, R^{4.3} is —CH₂Cl. In embodiments, R^{4.3} is —CH₂Br. In embodiments, R^{4.3} is —CH₂F.

In embodiments, R^{4.3} is —CH₂I. In embodiments, R^{4.3} is —CHCl₂. In embodiments, R^{4.3} is —CHBr₂. In embodiments, R^{4.3} is —CHF₂. In embodiments, R^{4.3} is —CHI₂. In embodiments, R^{4.3} is —CN. In embodiments, R^{4.3} is —OH. In embodiments, R^{4.3} is —NH₂. In embodiments, R^{4.3} is —COOH. In embodiments, R^{4.3} is —CONH₂. In embodiments, R^{4.3} is —NO₂. In embodiments, R^{4.3} is —SH. In embodiments, R^{4.3} is —SO₃H. In embodiments, R^{4.3} is —OSO₃H. In embodiments, R^{4.3} is —SO₂NH₂. In embodiments, R^{4.3} is —NHNH₂. In embodiments, R^{4.3} is —ONH₂. In embodiments, R^{4.3} is —NHC(O)NH₂. In embodiments, R^{4.3} is —NHSO₂H. In embodiments, R^{4.3} is —NHC(O)H. In embodiments, R^{4.3} is —NHC(O)OH. In embodiments, R^{4.3} is —NHOH. In embodiments, R^{4.3} is —OCCl₃. In embodiments, R^{4.3} is —OCBr₃. In embodiments, R^{4.3} is —OCF₃. In embodiments, R^{4.3} is —OCl₃. In embodiments, R^{4.3} is —OCH₂Cl. In embodiments, R^{4.3} is —OCH₂Br. In embodiments, R^{4.3} is —OCH₂F. In embodiments, R^{4.3} is —OCH₂I. In embodiments, R^{4.3} is —OCHCl₂. In embodiments, R^{4.3} is —OCHBr₂. In embodiments, R^{4.3} is —OCHF₂. In embodiments, R^{4.3} is —OCHI₂. In embodiments, R^{4.3} is —SF₅. In embodiments, R⁴₃ is —N₃. In embodiments, R^{4.3} is unsubstituted C₁-C₄ alkyl. In embodiments, R^{4.3} is unsubstituted methyl. In embodiments, R^{4.3} is unsubstituted ethyl. In embodiments, R^{4.3} is unsubstituted propyl. In embodiments, R^{4.3} is unsubstituted n-propyl. In embodiments, R^{4.3} is unsubstituted isopropyl. In embodiments, R^{4.3} is unsubstituted butyl. In embodiments, R^{4.3} is unsubstituted n-butyl. In embodiments, R^{4.3} is unsubstituted isobutyl. In embodiments, R^{4.3} is unsubstituted tert-butyl. In embodiments, R^{4.3} is unsubstituted 2 to 6 membered heteroalkyl. In embodiments, R^{4.3} is unsubstituted methoxy. In embodiments, R^{4.3} is unsubstituted ethoxy. In embodiments, R⁴is unsubstituted propoxy. In embodiments, R⁴is unsubstituted n-propoxy. In embodiments, R^{4.3} is unsubstituted isopropoxy. In embodiments, R^{4.3} is unsubstituted butoxy.

In embodiments, R^{4.3} is halogen, —CX⁴₃, or unsubstituted C₁-C₄ alkyl. In embodiments, R⁴₃ is —F, —Cl, —CF₃, or unsubstituted methyl. In embodiments, R^{4.3} is —F or —Cl.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl (e.g., C₁-C₅, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In embodiments, R⁵ is hydrogen. In embodiments, R⁵ is halogen. In embodiments, R⁵ is —F. In embodiments, R⁵ is —Cl. In embodiments, R⁵ is —Br. In embodiments, R⁵ is —I. In embodiments, R⁵ is —CCl₃. In embodiments, R⁵ is —CBr₃. In embodiments, R⁵ is —CF₃. In embodiments, R⁵ is —Cl₃. In embodiments, R⁵ is —CH₂Cl. In embodiments, R⁵ is —CH₂Br. In embodiments, R⁵ is —CH₂F. In embodiments, R⁵ is —CH₂I. In embodiments, R⁵ is —CHCl₂. In embodiments, R⁵ is —CHBr₂. In embodiments, R⁵ is —CHF₂. In embodiments, R⁵ is —CHI₂. In embodiments, R⁵ is —CN. In embodiments, R⁵ is —OH. In embodiments, R⁵ is —NH₂. In embodiments, R⁵ is —COOH. In embodiments, R⁵ is —CONH₂. In embodiments, R⁵ is —NO₂. In embodiments, R⁵ is —SH. In embodiments, R⁵ is —SO₃H. In embodiments, R⁵ is —OSO₃H. In embodiments, R⁵ is —SO₂NH₂. In embodiments, R⁵ is —NHNH₂. In embodiments, R⁵ is —ONH₂.

In embodiments, R⁵ is —NHC(O)NH₂. In embodiments, R⁵ is —NHSO₂H. In embodiments, R⁵ is —NHC(O)H. In embodiments, R⁵ is —NHC(O)OH. In embodiments, R⁵ is —NHOH. In embodiments, R⁵ is —OCCl₃. In embodiments, R⁵ is —OCBr₃. In embodiments, R⁵ is —OCF₃. In embodiments, R⁵ is —OCl₃. In embodiments, R⁵ is —OCH₂Cl. In embodiments, R⁵ is —OCH₂Br.

In embodiments, R⁵ is —OCH₂F. In embodiments, R⁵ is —OCH₂I. In embodiments, R⁵ is —OCHCl₂. In embodiments, R⁵ is —OCHBr₂. In embodiments, R⁵ is —OCHF₂. In embodiments, R⁵ is —OCHI₂. In embodiments, R⁵ is —SF₅. In embodiments, R⁵ is —N₃. In embodiments, R⁵ is unsubstituted C₁-C₄ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl. In embodiments, R⁵ is unsubstituted 2 to 6 membered heteroalkyl. In embodiments, R⁵ is unsubstituted methoxy. In embodiments, R⁵ is unsubstituted ethoxy. In embodiments, R⁵ is unsubstituted propoxy. In embodiments, R⁵ is unsubstituted n-propoxy. In embodiments, R⁵ is unsubstituted isopropoxy.

In embodiments, R⁵ is unsubstituted butoxy. In embodiments, R⁵ is unsubstituted C₃-C₈ cycloalkyl. In embodiments, R⁵ is unsubstituted cyclopropyl. In embodiments, R⁵ is unsubstituted cyclobutyl. In embodiments, R⁵ is unsubstituted cyclopentyl. In embodiments, R⁵ is unsubstituted cyclohexyl.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, R^{5A} is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl (e.g., C₁-C₈, C₁-C₆, or C₁-C₄), substituted or unsubstituted heteroalkyl (e.g., 2 to 8 membered, 2 to 6 membered, or 2 to 4 membered), substituted or unsubstituted cycloalkyl (e.g., C₃-C₈, C₃-C₆, or C₅-C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered).

In embodiments, R^{5A} is hydrogen. In embodiments, R^{5A} is halogen. In embodiments, R^{5A} is —F. In embodiments, R^{5A} is —Cl. In embodiments, R^{5A} is —Br. In embodiments, R^{5A} is —I.

In embodiments, R^{5A} is —CCl₃. In embodiments, R^{5A} is —CBr₃. In embodiments, R^{5A} is —CF₃. In embodiments, R^{5A} is —Cl₃. In embodiments, R^{5A} is —CH₂Cl. In embodiments, R^{5A} is —CH₂Br. In embodiments, R^{5A} is —CH₂F. In embodiments, R^{5A} is —CH₂I. In embodiments, R^{5A} is —CHCl₂. In embodiments, R^{5A} is —CHBr₂. In embodiments, R^{5A} is —CHF₂. In embodiments, R^{5A} is —CHI₂. In embodiments, R^{5A} is —CN. In embodiments, R^{5A} is —OH. In embodiments, R^{5A} is —NH₂. In embodiments, R^{5A} is —COOH. In embodiments, R^{5A} is —CONH₂. In embodiments, R^{5A} is —NO₂.

In embodiments, R^{5A} is —SH. In embodiments, R^{5A} is —SO₃H. In embodiments, R^{5A} is —OSO₃H.

In embodiments, R^{5A} is —SO₂NH₂. In embodiments, R^{5A} is —NHNH₂. In embodiments, R^{5A} is —ONH₂. In embodiments, R^{5A} is —NHC(O)NH₂. In embodiments, R^{5A} is —NHSO₂H. In embodiments, R^{5A} is —NHC(O)H. In embodiments, R^{5A} is —NHC(O)OH. In embodiments, R^{5A} is —NHOH. In embodiments, R^{5A} is —OCCl₃. In embodiments, R^{5A} is —OCBr₃. In embodiments, R^{5A} is —OCF₃. In embodiments, R^{5A} is —OCl₃. In embodiments, R^{5A} is —OCH₂Cl. In embodiments, R^{5A} is —OCH₂Br. In embodiments, R^{5A} is —OCH₂F. In embodiments, R^{5A} is —OCH₂I. In embodiments, R^{5A} is —OCHCl₂. In embodiments, R^{5A} is —OCHBr₂. In embodiments, R″ is —OCHF₂. In embodiments, R^{5A} is —OCHI₂. In embodiments, R^{5A} is —SF₅. In embodiments, R^{5A} is —N₃. In embodiments, R^{5A} is unsubstituted C₁-C₄ alkyl. In embodiments, R^{5A} is unsubstituted methyl. In embodiments, R^{5A} is unsubstituted ethyl. In embodiments, R^{5A} is unsubstituted propyl. In embodiments, R^{5A} is unsubstituted n-propyl. In embodiments, R^{5A} is unsubstituted isopropyl. In embodiments, R^{5A} is unsubstituted butyl. In embodiments, R^{5A} is unsubstituted n-butyl. In embodiments, R^{5A} is unsubstituted isobutyl. In embodiments, R^{5A} is unsubstituted tert-butyl. In embodiments, R^{5A} is unsubstituted 2 to 6 membered heteroalkyl. In embodiments, R^{5A} is unsubstituted methoxy. In embodiments, R^{5A} is unsubstituted ethoxy. In embodiments, R^{5A} is unsubstituted propoxy. In embodiments, R^{5A} is unsubstituted n-propoxy. In embodiments, R^{5A} is unsubstituted isopropoxy. In embodiments, R^{5A} is unsubstituted butoxy. In embodiments, R^{5A} is unsubstituted C₃-C₅ cycloalkyl. In embodiments, R^{5A} is unsubstituted cyclopropyl. In embodiments, R^{5A} is unsubstituted cyclobutyl. In embodiments, R^{5A} is unsubstituted cyclopentyl. In embodiments, R^{5A} is unsubstituted cyclohexyl.

In embodiments, a substituted ring formed when R¹ and R⁵ substituents are joined (e.g., substituted aryl and/or substituted heteroaryl) is substituted with at least one substituent group, size-limited substituent group, or lower substituent group; wherein if the substituted ring formed when R¹ and R⁵ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R¹ and R⁵ substituents are joined is substituted, it is substituted with at least one substituent group. In embodiments, when the substituted ring formed when R¹ and R⁵ substituents are joined is substituted, it is substituted with at least one size-limited substituent group. In embodiments, when the substituted ring formed when R¹ and R⁵ substituents are joined is substituted, it is substituted with at least one lower substituent group.

In embodiments, R¹ and R⁵ substituents are joined to form a substituted or unsubstituted phenyl or substituted or unsubstituted 5 to 6 membered heteroaryl. In embodiments, R¹ and R⁵ substituents are joined to form a substituted or unsubstituted phenyl. In embodiments, R¹ and R⁵ substituents are joined to form a substituted or unsubstituted 5 to 6 membered heteroaryl.

In embodiments, when R¹ is substituted, R¹ is substituted with one or more first substituent groups denoted by R″′ as explained in the definitions section above in the description of “first substituent group(s)”. In embodiments, when an R^{1.1} substituent group is substituted, the R^{1.1} substituent group is substituted with one or more second substituent groups denoted by R² as explained in the definitions section above in the description of “first substituent group(s)”.

In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R″, R^{1.2}, and R″ have values corresponding to the values of R^{WW}, R^{WW.2}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2} and R^{1.3}, respectively.

In embodiments, when two R¹ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.2}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R″, R^{1.2} and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, 20 R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents that are bonded to the same nitrogen atom are joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1A} and R^{1B} substituents that are bonded to the same nitrogen atom are joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.2}, R^{B.2}, and R^{1B.3} have values corresponding to the values of R^{WW} R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”.

In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R³₃ have values corresponding to the values of R^{WW}, R^{WW.l}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.2}, R^{3.2} and R³₃, respectively.

In embodiments, when two R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R³₃ have values corresponding to the values of R^{WW}, R^{WW.l}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.2}, R^{3.2} and R³₃, respectively.

In embodiments, when R^{3.1} is substituted, R^{3.1} is substituted with one or more first substituent groups denoted by R^{3.1.1} as explained in the definitions section above in the description of “first substituent group(s)”. In embodiments, when an R^{3.1.1} substituent group is substituted, the R^{3.1.1} substituent group is substituted with one or more second substituent groups denoted by R^{3.1.2} as explained in the definitions section above in the description of “first substituent group(s)”. In embodiments, when an R^{3.1.2} substituent group is substituted, the R^{3.1.2} substituent group is substituted with one or more third substituent groups denoted by R^{3.1.3} as explained in the definitions section above in the description of “first substituent group(s)”. In the above embodiments, R^{3.1}, R^{3.1.1}, R^{3.1.2}, and R^{3.1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.2}, R^{3.1}, R^{3.1.2}, and R^{3.1.2}, respectively.

In embodiments, when R^{3.2} is substituted, R^{3.2} is substituted with one or more first substituent groups denoted by R^{3.2.1} as explained in the definitions section above in the description of “first substituent group(s)”. In embodiments, when an R^{3.2.1} substituent group is substituted, the R^{3.2.1} substituent group is substituted with one or more second substituent groups denoted by R^{3.2.2} as explained in the definitions section above in the description of “first substituent group(s)”. In embodiments, when an R^{3.2.2} substituent group is substituted, the R^{3.2.2} substituent group is substituted with one or more third substituent groups denoted by R^{3.2.3} as explained in the definitions section above in the description of “first substituent group(s)”. In the above embodiments, R^{3.2}, R^{3.2.1}, R^{3.2.2}, and R^{3.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3.2}, R^{3.2.1}, R^{3.2.2}, and R^{3.2.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A}. substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3A} and R^{3B} substituents that are bonded to the same nitrogen atom are joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3A} and R^{3B} substituents that are bonded to the same nitrogen atom are joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above 5 embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to 20 R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW}, R^{WW.2}, and R^{WW.3} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”.

In embodiments, when an R^{4.2} substituent group is substituted, the R^{4.2} substituent group is substituted with one or more third substituent groups denoted by R⁴₃ as explained in the definitions section above in the description of “first substituent group(s)”. In the above embodiments, R⁴, R^{4.1}, R^{4.2}, and R^{4.3} have values corresponding to the values of R^{WW}, R^{WW.l}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.2}, R^{4.2} and R^{4.3}, respectively.

In embodiments, when two R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R⁴₃ as explained in the definitions section above in the description of “first substituent group(s)”. In the above embodiments, R⁴, R^{4.1}, R^{4.2}, and R^{4.3} have values corresponding to the values of R^{WW}, R^{WW.l}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.2}, R^{4.2} and R^{4.3}, respectively.

In embodiments, when R^{4.1} is substituted, R^{4.1} is substituted with one or more first substituent groups denoted by R^{4.11} as explained in the definitions section above in the description of “first substituent group(s)”. In embodiments, when an R^{4.11} substituent group is substituted, the R^{4.11} substituent group is substituted with one or more second substituent groups denoted by R^{4.1.2} as explained in the definitions section above in the description of “first substituent group(s)”. In embodiments, when an R^{4.1.2} substituent group is substituted, the R^{4.1.2} substituent group is substituted with one or more third substituent groups denoted by R^{4.13} as explained in the definitions section above in the description of “first substituent group(s)”. In the above embodiments, R^{4.2}, R^{4.1}, R^{4.1.2}, and R^{4.13} have values corresponding to the values of R^{WW}, R^{WW.1}, R^{WW.2}, and R^{WW}″, respectively, as explained in the definitions section above in the description of “first substituent group(s)”, wherein R^{WW}, R^{WW.1}, R^{WW.2}, and R^{WW.3} correspond to R^{4.2}, R^{4.1}, R^{4.1.2}, and R^{4.1}, respectively.

In embodiments, when R^{4.3} is substituted, R^{4.3} is substituted with one or more first substituent groups denoted by R^{4.3.1} as explained in the definitions section above in the description of “first substituent group(s)”. In embodiments, when an R^{4.3.1} substituent group is substituted, the R^{4.3.1} substituent group is substituted with one or more second substituent groups denoted by R^{4.3.2} as explained in the definitions section above in the description of “first substituent group(s)”. In embodiments, when an R^{4.3.2} substituent group is substituted, the R^{4.3.2} substituent group is substituted with one or more third substituent groups denoted by R^{4.3.3} as explained in the definitions section above in the description of “first substituent group(s)”. In the above embodiments, R^{4.3}, R^{4.3}, R^{4.3.2}, and R^{4.3.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.3} correspond to R⁴₃, R^{4.3}, R^{4.3.2}, and R^{4.3.2}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R⁴A. substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R⁴B is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4A} and R^{4B} substituents that are bonded to the same nitrogen atom are joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4A} and R^{4B} substituents that are bonded to the same nitrogen atom are joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D.1}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.2}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.2}, R^{5.2} and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW}, R^{WW.2}, and R^{WW.3} correspond to R^{5A}, R^{5A.1} R^{5A.2} and R^{5A.3}, respectively.

In embodiments, when R¹ and R⁵ substituents are joined to form a moiety that is substituted (e.g., a substituted aryl or substituted heteroaryl), the moiety is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R¹ and R⁵ substituents that are bonded to the same nitrogen atom are joined to form a moiety that is substituted (e.g., a substituted aryl or substituted heteroaryl), the moiety is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R¹⁰ substituent group is substituted, the R¹⁰ substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R¹⁰, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁰, R^{10.1}, R^{10.2}, and R^{10.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW3}, respectively, as explained in the definitions section above in the description of “first substituent group(s)”, wherein L^{WW}, R^{LWW.1}, R^{LWW.2}, and R^{LWW3} are L¹, R^{L1.2}, R^{L1.2}, and R^{L1.2}, respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L² R^{L20.1}, R^{L2.2}, and R^{L2.3}, respectively.

In embodiments, when L³ is substituted, L³ is substituted with one or more first substituent groups denoted by R^{L3}. as explained in the definitions section above in the description of “first substituent group(s)”. In embodiments, when an R^{L3} substituent group is substituted, the R^{L3} substituent group is substituted with one or more second substituent groups denoted by R^{L3.2} as explained in the definitions section above in the description of “first substituent group(s)”. In embodiments, when an R^{L3.2} substituent group is substituted, the R^{L3.2} substituent group is substituted with one or more third substituent groups denoted by R^{L3.3} as explained in the definitions section above in the description of “first substituent group(s)”. In the above embodiments, L³, R^{L3}, R^{L3.2}, and R^{L3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L³, R^{L3.2}, R^{L3.2}, and R^{L3.3}, respectively.

In embodiments, the compound has the formula:

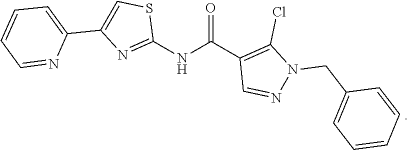 [SMILES: O=C(Nc1nc(-c2ccccn2)cs1)c1cnn(Cc2ccccc2)c1Cl]

In embodiments, the compound has the formula:

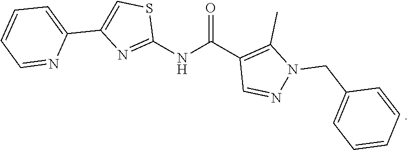 [SMILES: Cc1c(C(=O)Nc2nc(-c3ccccn3)cs2)cnn1Cc1ccccc1]

In embodiments, the compound has the formula:

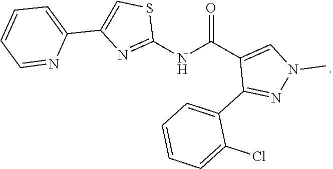 [SMILES: Cn1cc(C(=O)Nc2nc(-c3ccccn3)cs2)c(-c2ccccc2Cl)n1]

In embodiments, the compound has the formula:

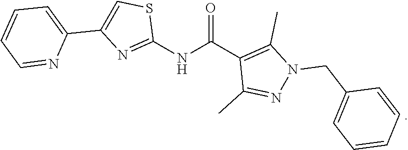 [SMILES: Cc1nn(Cc2ccccc2)c(C)c1C(=O)Nc1nc(-c2ccccn2)cs1]

In embodiments, the compound has the formula:

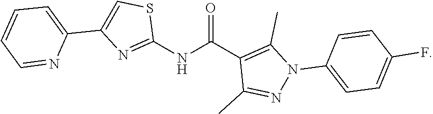 [SMILES: Cc1nn(-c2ccc(F)cc2)c(C)c1C(=O)Nc1nc(-c2ccccn2)cs1]

In embodiments, the compound has the formula:

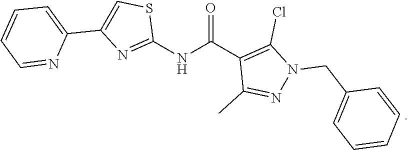 [SMILES: Cc1nn(Cc2ccccc2)c(Cl)c1C(=O)Nc1nc(-c2ccccn2)cs1]

In embodiments, the compound has the formula:

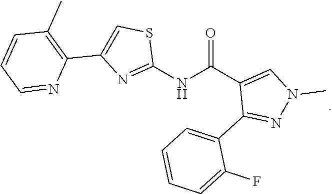 [SMILES: Cc1cccnc1-c1csc(NC(=O)c2cn(C)nc2-c2ccccc2F)n1]

In embodiments, the compound has the formula:

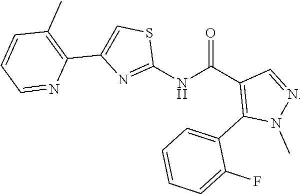 [SMILES: Cc1cccnc1-c1csc(NC(=O)c2cnn(C)c2-c2ccccc2F)n1]

In embodiments, the compound has the formula:

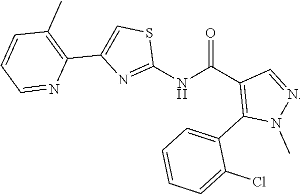 [SMILES: Cc1cccnc1-c1csc(NC(=O)c2cnn(C)c2-c2ccccc2Cl)n1]

In embodiments, the compound has the formula:

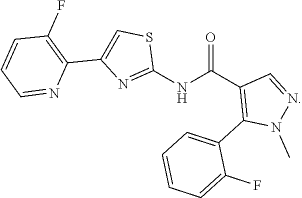 [SMILES: Cn1ncc(C(=O)Nc2nc(-c3ncccc3F)cs2)c1-c1ccccc1F]

In embodiments, the compound has the formula:

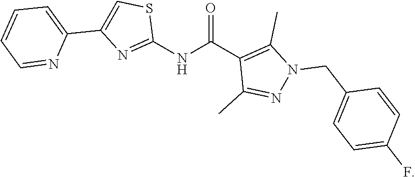 [SMILES: Cc1nn(Cc2ccc(F)cc2)c(C)c1C(=O)Nc1nc(-c2ccccn2)cs1]

In embodiments, the compound has the formula:

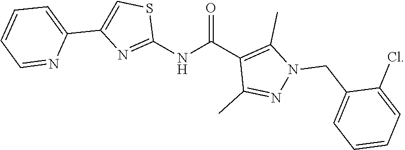 [SMILES: Cc1nn(Cc2ccccc2Cl)c(C)c1C(=O)Nc1nc(-c2ccccn2)cs1]

In embodiments, the compound has the formula:

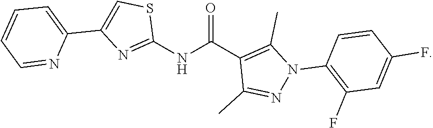 [SMILES: Cc1nn(-c2ccc(F)cc2F)c(C)c1C(=O)Nc1nc(-c2ccccn2)cs1]

In embodiments, the compound has the formula:

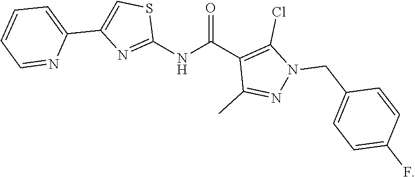 [SMILES: Cc1nn(Cc2ccc(F)cc2)c(Cl)c1C(=O)Nc1nc(-c2ccccn2)cs1]

In embodiments, the compound has the formula:

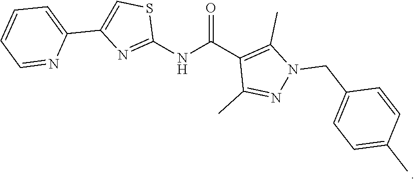 [SMILES: Cc1ccc(Cn2nc(C)c(C(=O)Nc3nc(-c4ccccn4)cs3)c2C)cc1]

In embodiments, the compound has the formula:

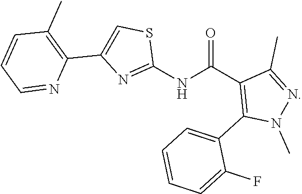 [SMILES: Cc1cccnc1-c1csc(NC(=O)c2c(C)nn(C)c2-c2ccccc2F)n1]

In embodiments, the compound has the formula:

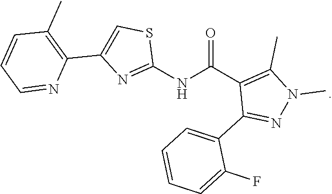 [SMILES: Cc1cccnc1-c1csc(NC(=O)c2c(-c3ccccc3F)nn(C)c2C)n1]

In embodiments, the compound has the formula:

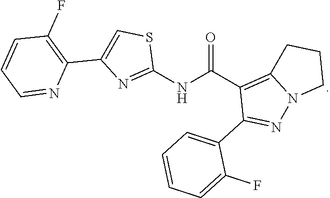 [SMILES: O=C(Nc1nc(-c2ncccc2F)cs1)c1c(-c2ccccc2F)nn2c1CCC2]

In embodiments, the compound has the formula:

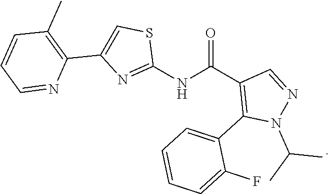 [SMILES: Cc1cccnc1-c1csc(NC(=O)c2cnn(C(C)C)c2-c2ccccc2F)n1]

In embodiments, the compound has the formula:

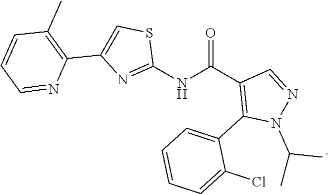 [SMILES: Cc1cccnc1-c1csc(NC(=O)c2cnn(C(C)C)c2-c2ccccc2Cl)n1]

In embodiments, the compound has the formula:

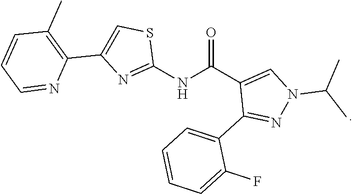 [SMILES: Cc1cccnc1-c1csc(NC(=O)c2cn(C(C)C)nc2-c2ccccc2F)n1]

In embodiments, the compound has the formula:

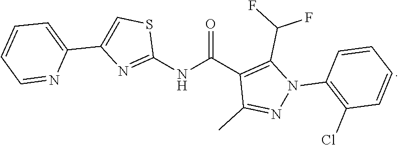 [SMILES: Cc1nn(-c2ccccc2Cl)c(C(F)F)c1C(=O)Nc1nc(-c2ccccn2)cs1]

In embodiments, the compound has the formula:

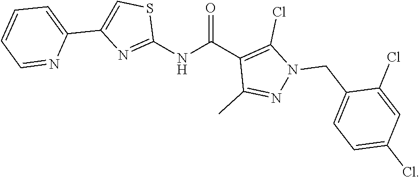 [SMILES: Cc1nn(Cc2ccc(Cl)cc2Cl)c(Cl)c1C(=O)Nc1nc(-c2ccccn2)cs1]

In embodiments, the compound has the formula:

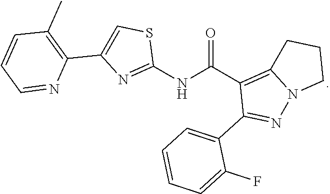 [SMILES: Cc1cccnc1-c1csc(NC(=O)c2c(-c3ccccc3F)nn3c2CCC3)n1]

In embodiments, the compound has the formula:

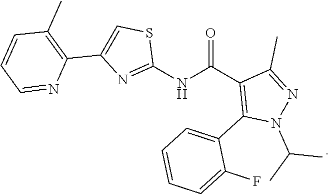 [SMILES: Cc1cccnc1-c1csc(NC(=O)c2c(C)nn(C(C)C)c2-c2ccccc2F)n1]

In embodiments, the compound has the formula:

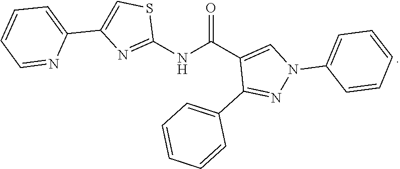 [SMILES: O=C(Nc1nc(-c2ccccn2)cs1)c1cn(-c2ccccc2)nc1-c1ccccc1]

In embodiments, the compound has the formula:

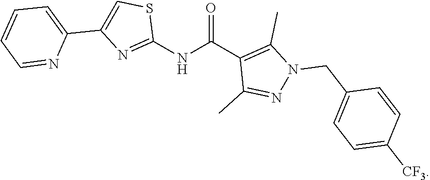 [SMILES: Cc1nn(Cc2ccc(C(F)(F)F)cc2)c(C)c1C(=O)Nc1nc(-c2ccccn2)cs1]

In embodiments, the compound has the formula:

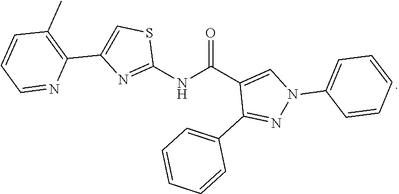 [SMILES: Cc1cccnc1-c1csc(NC(=O)c2cn(-c3ccccc3)nc2-c2ccccc2)n1]

In embodiments, the compound has the formula:

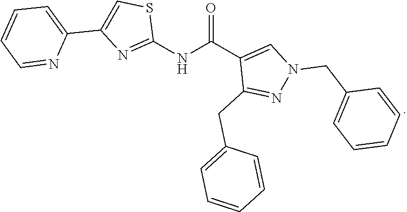 [SMILES: O=C(Nc1nc(-c2ccccn2)cs1)c1cn(Cc2ccccc2)nc1Cc1ccccc1]

In embodiments, the compound has the formula:

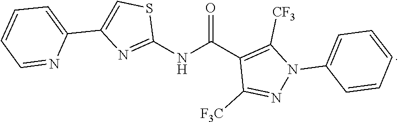 [SMILES: O=C(Nc1nc(-c2ccccn2)cs1)c1c(C(F)(F)F)nn(-c2ccccc2)c1C(F)(F)F]

In embodiments, the compound has the formula:

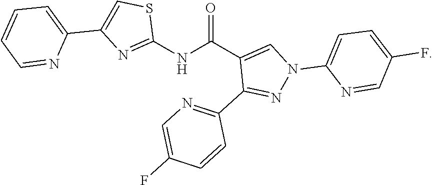 [SMILES: O=C(Nc1nc(-c2ccccn2)cs1)c1cn(-c2ccc(F)cn2)nc1-c1ccc(F)cn1]

In embodiments, the compound has the formula:

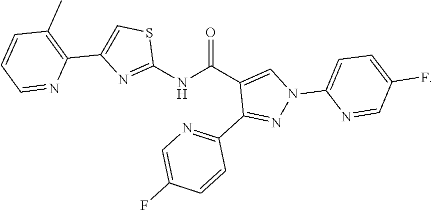 [SMILES: Cc1cccnc1-c1csc(NC(=O)c2cn(-c3ccc(F)cn3)nc2-c2ccc(F)cn2)n1]

In embodiments, the compound has the formula:

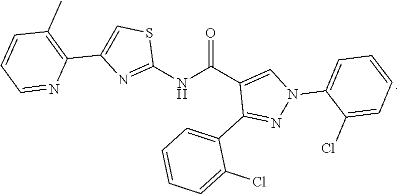 [SMILES: Cc1cccnc1-c1csc(NC(=O)c2cn(-c3ccccc3Cl)nc2-c2ccccc2Cl)n1]

In embodiments, the compound has the formula:

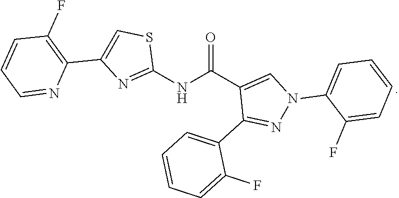 [SMILES: O=C(Nc1nc(-c2ncccc2F)cs1)c1cn(-c2ccccc2F)nc1-c1ccccc1F]

In embodiments, the compound has the formula:

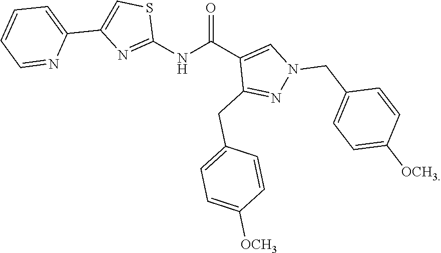 [SMILES: COc1ccc(Cc2nn(Cc3ccc(OC)cc3)cc2C(=O)Nc2nc(-c3ccccn3)cs2)cc1]

In embodiments, the compound has the formula:

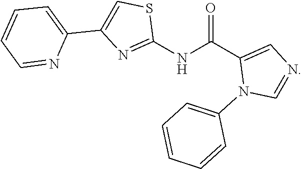 [SMILES: O=C(Nc1nc(-c2ccccn2)cs1)c1cncn1-c1ccccc1]

In embodiments, the compound has the formula:

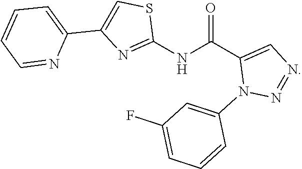 [SMILES: O=C(Nc1nc(-c2ccccn2)cs1)c1cnnn1-c1cccc(F)c1]

In embodiments, the compound has the formula: In

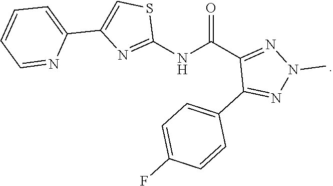 [SMILES: Cn1nc(C(=O)Nc2nc(-c3ccccn3)cs2)c(-c2ccc(F)cc2)n1]

In embodiments, the compound has the formula:

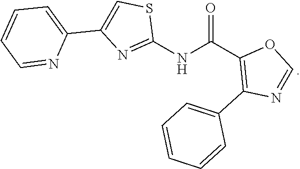 [SMILES: O=C(Nc1nc(-c2ccccn2)cs1)c1ocnc1-c1ccccc1]

In embodiments, the compound has the formula:

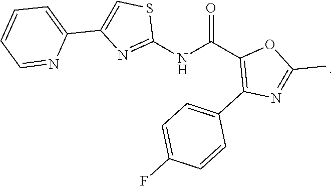 [SMILES: Cc1nc(-c2ccc(F)cc2)c(C(=O)Nc2nc(-c3ccccn3)cs2)o1]

In embodiments, the compound has the formula:

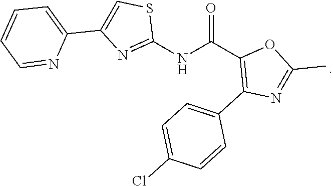 [SMILES: Cc1nc(-c2ccc(Cl)cc2)c(C(=O)Nc2nc(-c3ccccn3)cs2)o1]

In embodiments, the compound has the formula:

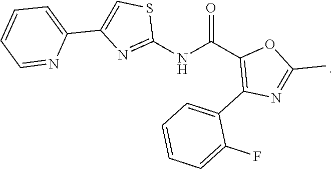 [SMILES: Cc1nc(-c2ccccc2F)c(C(=O)Nc2nc(-c3ccccn3)cs2)o1]

In embodiments, the compound has the formula:

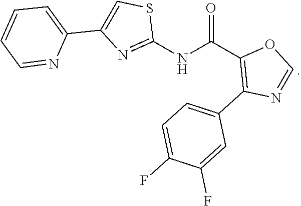 [SMILES: O=C(Nc1nc(-c2ccccn2)cs1)c1ocnc1-c1ccc(F)c(F)c1]

In embodiments, the compound has the formula:

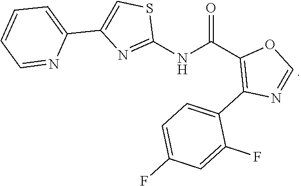 [SMILES: O=C(Nc1nc(-c2ccccn2)cs1)c1ocnc1-c1ccc(F)cc1F]

In embodiments, the compound has the formula:

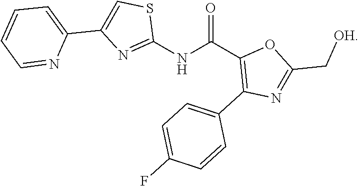 [SMILES: O=C(Nc1nc(-c2ccccn2)cs1)c1oc(CO)nc1-c1ccc(F)cc1]

In embodiments, the compound has the formula:

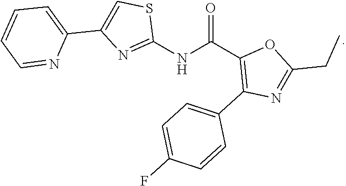 [SMILES: CCc1nc(-c2ccc(F)cc2)c(C(=O)Nc2nc(-c3ccccn3)cs2)o1]

In embodiments, the compound has the formula:

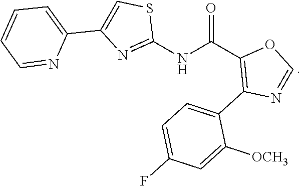 [SMILES: COc1cc(F)ccc1-c1ncoc1C(=O)Nc1nc(-c2ccccn2)cs1]

In embodiments, the compound has the formula:

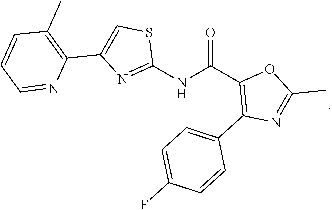 [SMILES: Cc1nc(-c2ccc(F)cc2)c(C(=O)Nc2nc(-c3ncccc3C)cs2)o1]

In embodiments, the compound has the formula:

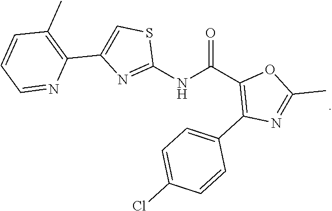 [SMILES: Cc1nc(-c2ccc(Cl)cc2)c(C(=O)Nc2nc(-c3ncccc3C)cs2)o1]

In embodiments, the compound has the formula:

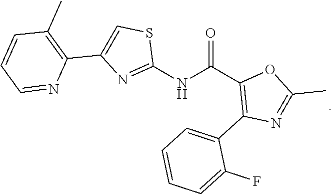 [SMILES: Cc1nc(-c2ccccc2F)c(C(=O)Nc2nc(-c3ncccc3C)cs2)o1]

In embodiments, the compound has the formula:

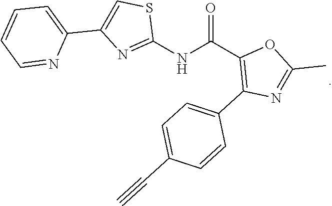 [SMILES: C#Cc1ccc(-c2nc(C)oc2C(=O)Nc2nc(-c3ccccn3)cs2)cc1]

In embodiments, the compound has the formula:

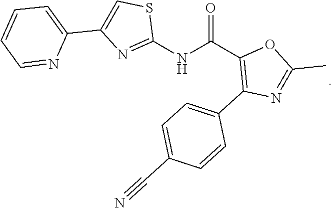 [SMILES: Cc1nc(-c2ccc(C#N)cc2)c(C(=O)Nc2nc(-c3ccccn3)cs2)o1]

In embodiments, the compound has the formula:

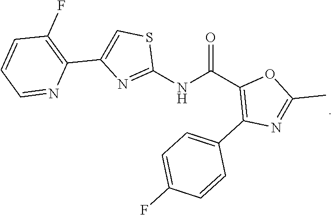 [SMILES: Cc1nc(-c2ccc(F)cc2)c(C(=O)Nc2nc(-c3ncccc3F)cs2)o1]

In embodiments, the compound has the formula:

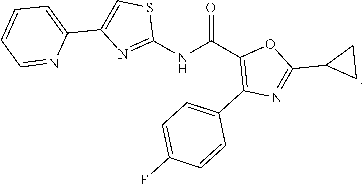 [SMILES: O=C(Nc1nc(-c2ccccn2)cs1)c1oc(C2CC2)nc1-c1ccc(F)cc1]

In embodiments, the compound has the formula:

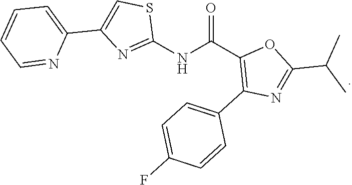 [SMILES: CC(C)c1nc(-c2ccc(F)cc2)c(C(=O)Nc2nc(-c3ccccn3)cs2)o1]

In embodiments, the compound has the formula:

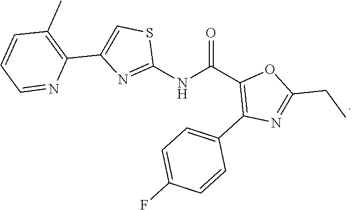 [SMILES: CCc1nc(-c2ccc(F)cc2)c(C(=O)Nc2nc(-c3ncccc3C)cs2)o1]

In embodiments, the compound has the formula:

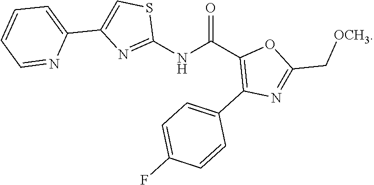 [SMILES: COCc1nc(-c2ccc(F)cc2)c(C(=O)Nc2nc(-c3ccccn3)cs2)o1]

In embodiments, the compound has the formula:

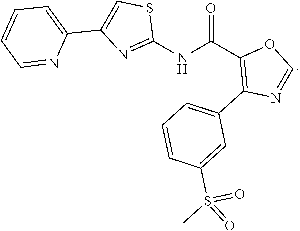 [SMILES: CS(=O)(=O)c1cccc(-c2ncoc2C(=O)Nc2nc(-c3ccccn3)cs2)c1]

In embodiments, the compound has the formula:

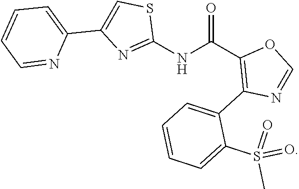 [SMILES: CS(=O)(=O)c1ccccc1-c1ncoc1C(=O)Nc1nc(-c2ccccn2)cs1]

In embodiments, the compound has the formula:

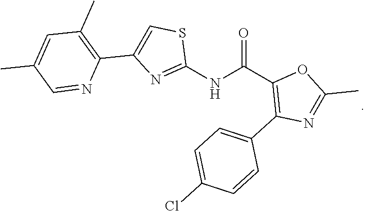

In embodiments, the compound has the formula:

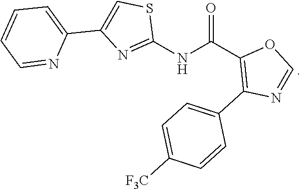 [SMILES: O=C(Nc1nc(-c2ccccn2)cs1)c1ocnc1-c1ccc(C(F)(F)F)cc1]

In embodiments, the compound has the formula:

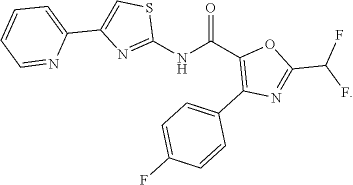 [SMILES: O=C(Nc1nc(-c2ccccn2)cs1)c1oc(C(F)F)nc1-c1ccc(F)cc1]

In embodiments, the compound has the formula:

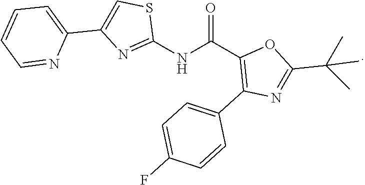 [SMILES: CC(C)(C)c1nc(-c2ccc(F)cc2)c(C(=O)Nc2nc(-c3ccccn3)cs2)o1]

In embodiments, the compound has the formula:

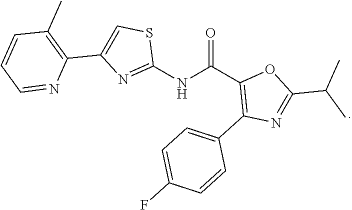 [SMILES: Cc1cccnc1-c1csc(NC(=O)c2oc(C(C)C)nc2-c2ccc(F)cc2)n1]

In embodiments, the compound has the formula:

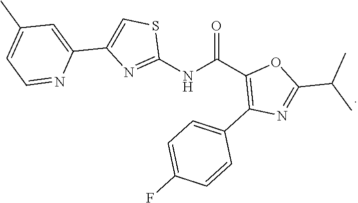 [SMILES: Cc1ccnc(-c2csc(NC(=O)c3oc(C(C)C)nc3-c3ccc(F)cc3)n2)c1]

In embodiments, the compound has the formula:

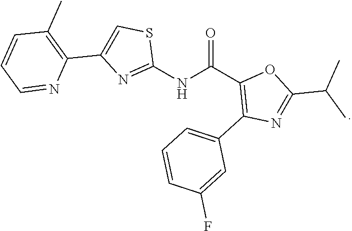 [SMILES: Cc1cccnc1-c1csc(NC(=O)c2oc(C(C)C)nc2-c2cccc(F)c2)n1]

In embodiments, the compound has the formula:

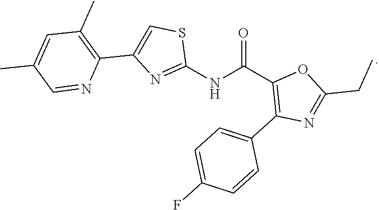 [SMILES: CCc1nc(-c2ccc(F)cc2)c(C(=O)Nc2nc(-c3ncc(C)cc3C)cs2)o1]

In embodiments, the compound has the formula:

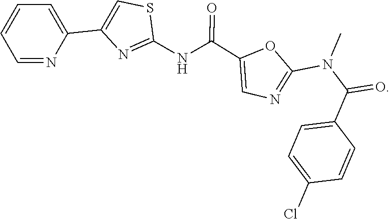 [SMILES: CN(C(=O)c1ccc(Cl)cc1)c1ncc(C(=O)Nc2nc(-c3ccccn3)cs2)o1]

In embodiments, the compound has the formula:

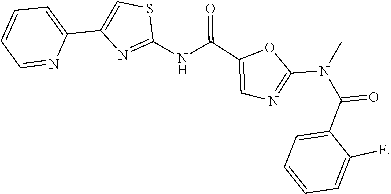 [SMILES: CN(C(=O)c1ccccc1F)c1ncc(C(=O)Nc2nc(-c3ccccn3)cs2)o1]

In embodiments, the compound has the formula:

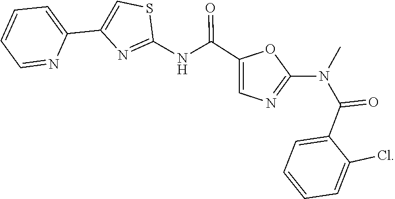 [SMILES: CN(C(=O)c1ccccc1Cl)c1ncc(C(=O)Nc2nc(-c3ccccn3)cs2)o1]

In embodiments, the compound has the formula:

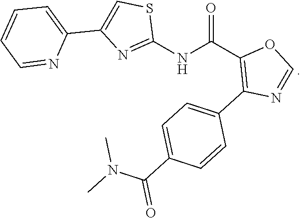 [SMILES: CN(C)C(=O)c1ccc(-c2ncoc2C(=O)Nc2nc(-c3ccccn3)cs2)cc1]

In embodiments, the compound has the formula:

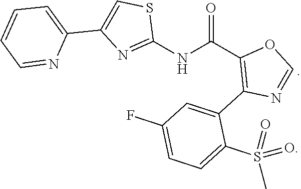 [SMILES: CS(=O)(=O)c1ccc(F)cc1-c1ncoc1C(=O)Nc1nc(-c2ccccn2)cs1]

In embodiments, the compound has the formula:

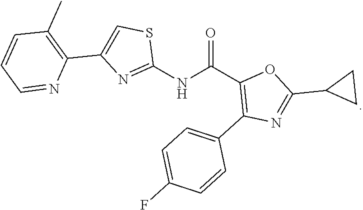 [SMILES: Cc1cccnc1-c1csc(NC(=O)c2oc(C3CC3)nc2-c2ccc(F)cc2)n1]

In embodiments, the compound has the formula:

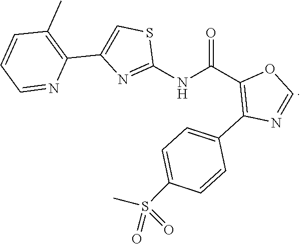 [SMILES: Cc1cccnc1-c1csc(NC(=O)c2ocnc2-c2ccc(S(C)(=O)=O)cc2)n1]

In embodiments, the compound has the formula:

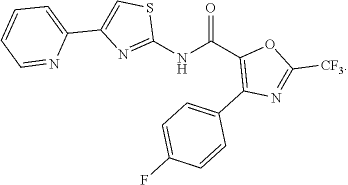 [SMILES: O=C(Nc1nc(-c2ccccn2)cs1)c1oc(C(F)(F)F)nc1-c1ccc(F)cc1]

In embodiments, the compound has the formula:

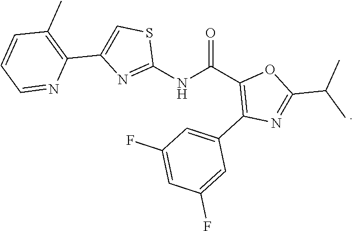 [SMILES: Cc1cccnc1-c1csc(NC(=O)c2oc(C(C)C)nc2-c2cc(F)cc(F)c2)n1]

In embodiments, the compound has the formula:

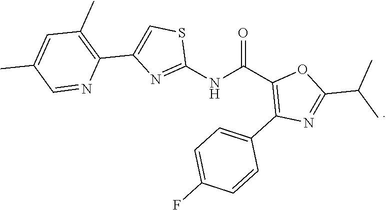 [SMILES: Cc1cnc(-c2csc(NC(=O)c3oc(C(C)C)nc3-c3ccc(F)cc3)n2)c(C)c1]

In embodiments, the compound has the formula:

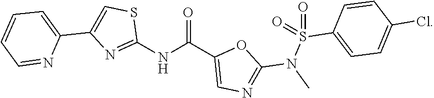 [SMILES: CN(c1ncc(C(=O)Nc2nc(-c3ccccn3)cs2)o1)S(=O)(=O)c1ccc(Cl)cc1]

In embodiments, the compound has the formula:

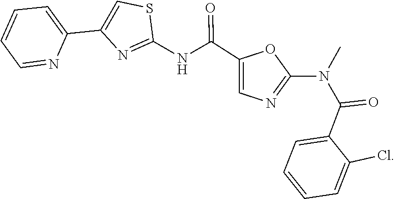 [SMILES: CN(C(=O)c1ccccc1Cl)c1ncc(C(=O)Nc2nc(-c3ccccn3)cs2)o1]

In embodiments, the compound has the formula:

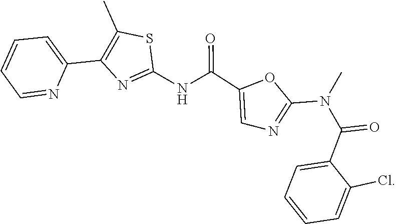 [SMILES: Cc1sc(NC(=O)c2cnc(N(C)C(=O)c3ccccc3Cl)o2)nc1-c1ccccn1]

In embodiments, the compound has the formula:

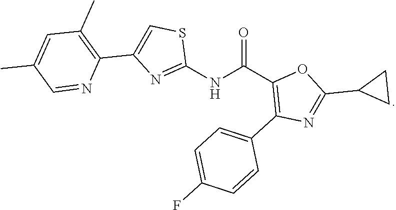 [SMILES: Cc1cnc(-c2csc(NC(=O)c3oc(C4CC4)nc3-c3ccc(F)cc3)n2)c(C)c1]

In embodiments, the compound has the formula:

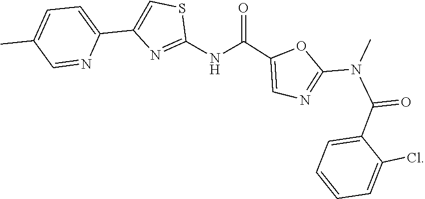 [SMILES: Cc1ccc(-c2csc(NC(=O)c3cnc(N(C)C(=O)c4ccccc4Cl)o3)n2)nc1]

In embodiments, the compound has the formula:

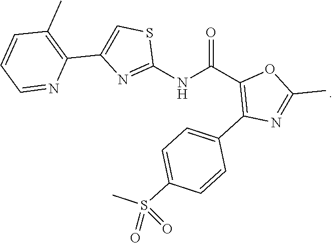 [SMILES: Cc1nc(-c2ccc(S(C)(=O)=O)cc2)c(C(=O)Nc2nc(-c3ncccc3C)cs2)o1]

In embodiments, the compound has the formula:

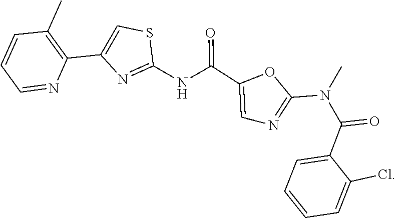 [SMILES: Cc1cccnc1-c1csc(NC(=O)c2cnc(N(C)C(=O)c3ccccc3Cl)o2)n1]

In embodiments, the compound has the formula:

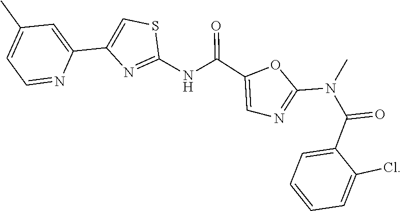 [SMILES: Cc1ccnc(-c2csc(NC(=O)c3cnc(N(C)C(=O)c4ccccc4Cl)o3)n2)c1]

In embodiments, the compound has the formula:

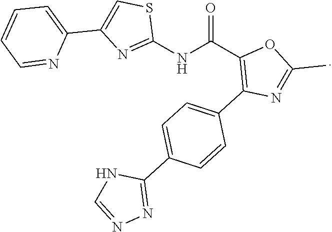 [SMILES: Cc1nc(-c2ccc(-c3nnc[nH]3)cc2)c(C(=O)Nc2nc(-c3ccccn3)cs2)o1]

In embodiments, the compound has the formula:

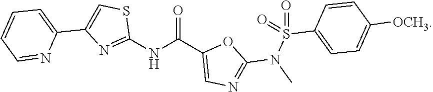 [SMILES: COc1ccc(S(=O)(=O)N(C)c2ncc(C(=O)Nc3nc(-c4ccccn4)cs3)o2)cc1]

In embodiments, the compound has the formula:

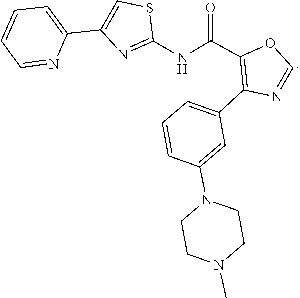 [SMILES: CN1CCN(c2cccc(-c3ncoc3C(=O)Nc3nc(-c4ccccn4)cs3)c2)CC1]

In embodiments, the compound has the formula:

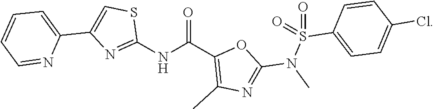 [SMILES: Cc1nc(N(C)S(=O)(=O)c2ccc(Cl)cc2)oc1C(=O)Nc1nc(-c2ccccn2)cs1]

In embodiments, the compound has the formula:

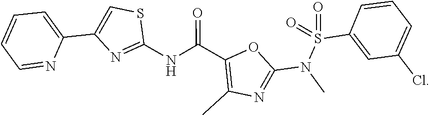 [SMILES: Cc1nc(N(C)S(=O)(=O)c2cccc(Cl)c2)oc1C(=O)Nc1nc(-c2ccccn2)cs1]

In embodiments, the compound has the formula:

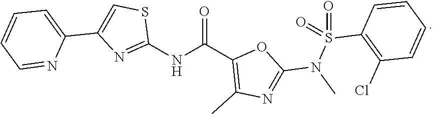 [SMILES: Cc1nc(N(C)S(=O)(=O)c2ccccc2Cl)oc1C(=O)Nc1nc(-c2ccccn2)cs1]

In embodiments, the compound has the formula:

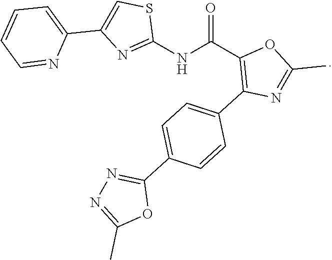 [SMILES: Cc1nnc(-c2ccc(-c3nc(C)oc3C(=O)Nc3nc(-c4ccccn4)cs3)cc2)o1]

In embodiments, the compound has the formula:

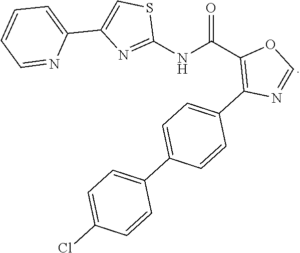 [SMILES: O=C(Nc1nc(-c2ccccn2)cs1)c1ocnc1-c1ccc(-c2ccc(Cl)cc2)cc1]

In embodiments, the compound has the formula:

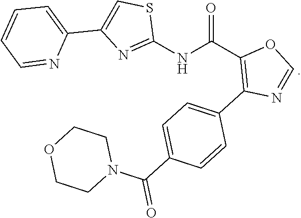 [SMILES: O=C(Nc1nc(-c2ccccn2)cs1)c1ocnc1-c1ccc(C(=O)N2CCOCC2)cc1]

In embodiments, the compound has the formula:

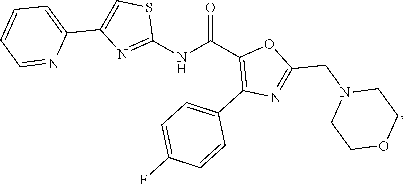 [SMILES: O=C(Nc1nc(-c2ccccn2)cs1)c1oc(CN2CCOCC2)nc1-c1ccc(F)cc1]

In embodiments, the compound has the formula:

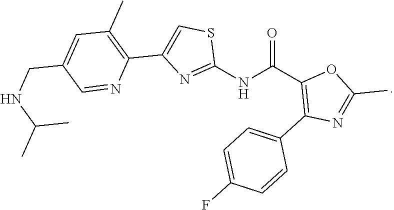 [SMILES: Cc1nc(-c2ccc(F)cc2)c(C(=O)Nc2nc(-c3ncc(CNC(C)C)cc3C)cs2)o1]

In embodiments, the compound has the formula:

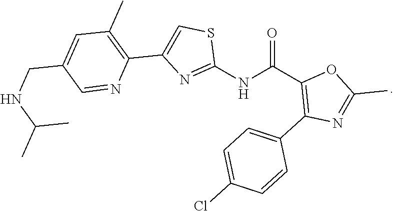 [SMILES: Cc1nc(-c2ccc(Cl)cc2)c(C(=O)Nc2nc(-c3ncc(CNC(C)C)cc3C)cs2)o1]

In embodiments, the compound has the formula:

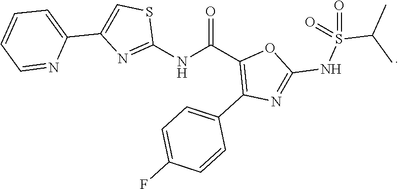 [SMILES: CC(C)S(=O)(=O)Nc1nc(-c2ccc(F)cc2)c(C(=O)Nc2nc(-c3ccccn3)cs2)o1]

In embodiments, the compound has the formula:

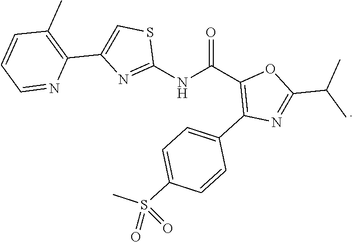 [SMILES: Cc1cccnc1-c1csc(NC(=O)c2oc(C(C)C)nc2-c2ccc(S(C)(=O)=O)cc2)n1]

In embodiments, the compound has the formula:

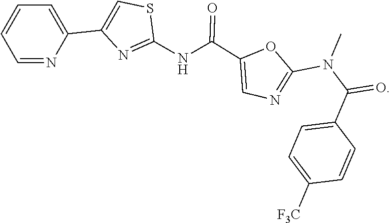 [SMILES: CN(C(=O)c1ccc(C(F)(F)F)cc1)c1ncc(C(=O)Nc2nc(-c3ccccn3)cs2)o1]

In embodiments, the compound has the formula:

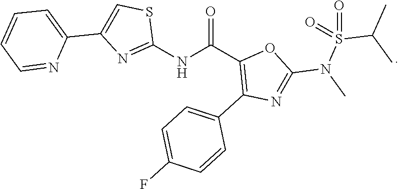 [SMILES: CC(C)S(=O)(=O)N(C)c1nc(-c2ccc(F)cc2)c(C(=O)Nc2nc(-c3ccccn3)cs2)o1]

In embodiments, the compound has the formula:

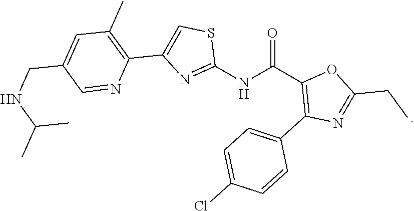 [SMILES: CCc1nc(-c2ccc(Cl)cc2)c(C(=O)Nc2nc(-c3ncc(CNC(C)C)cc3C)cs2)o1]

In embodiments, the compound has the formula:

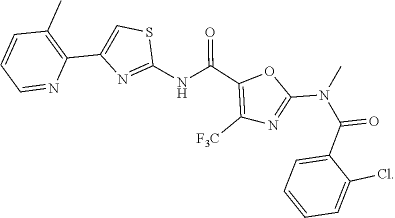 [SMILES: Cc1cccnc1-c1csc(NC(=O)c2oc(N(C)C(=O)c3ccccc3Cl)nc2C(F)(F)F)n1]

In embodiments, the compound has the formula:

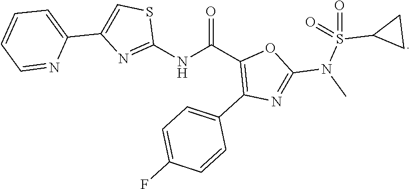 [SMILES: CN(c1nc(-c2ccc(F)cc2)c(C(=O)Nc2nc(-c3ccccn3)cs2)o1)S(=O)(=O)C1CC1]

In embodiments, the compound has the formula:

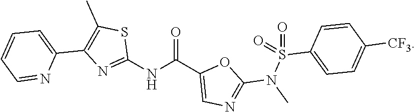 [SMILES: Cc1sc(NC(=O)c2cnc(N(C)S(=O)(=O)c3ccc(C(F)(F)F)cc3)o2)nc1-c1ccccn1]

In embodiments, the compound has the formula:

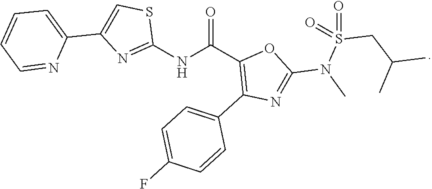 [SMILES: CC(C)CS(=O)(=O)N(C)c1nc(-c2ccc(F)cc2)c(C(=O)Nc2nc(-c3ccccn3)cs2)o1]

In embodiments, the compound has the formula:

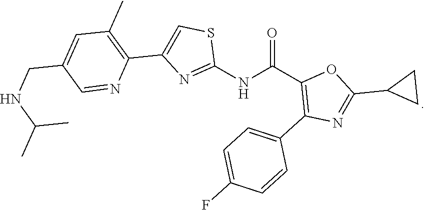 [SMILES: Cc1cc(CNC(C)C)cnc1-c1csc(NC(=O)c2oc(C3CC3)nc2-c2ccc(F)cc2)n1]

In embodiments, the compound has the formula:

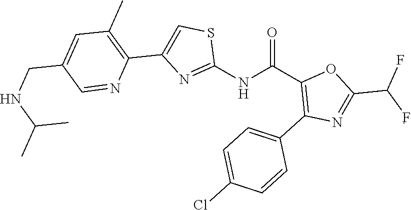 [SMILES: Cc1cc(CNC(C)C)cnc1-c1csc(NC(=O)c2oc(C(F)F)nc2-c2ccc(Cl)cc2)n1]

In embodiments, the compound has the formula:

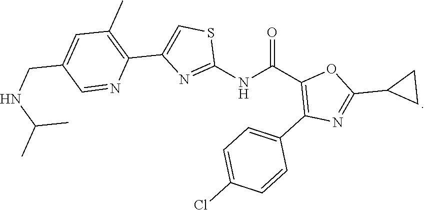 [SMILES: Cc1cc(CNC(C)C)cnc1-c1csc(NC(=O)c2oc(C3CC3)nc2-c2ccc(Cl)cc2)n1]

In embodiments, the compound has the formula:

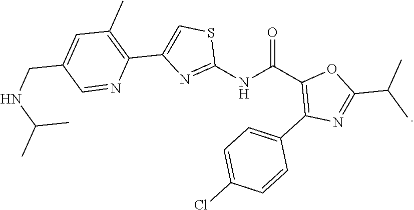 [SMILES: Cc1cc(CNC(C)C)cnc1-c1csc(NC(=O)c2oc(C(C)C)nc2-c2ccc(Cl)cc2)n1]

In embodiments, the compound has the formula:

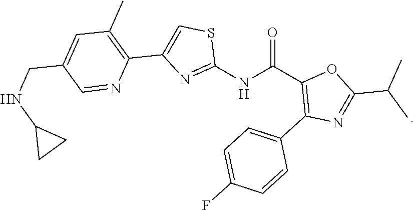 [SMILES: Cc1cc(CNC2CC2)cnc1-c1csc(NC(=O)c2oc(C(C)C)nc2-c2ccc(F)cc2)n1]

In embodiments, the compound has the formula

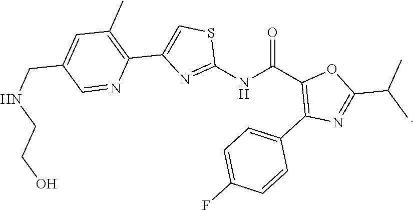 [SMILES: Cc1cc(CNCCO)cnc1-c1csc(NC(=O)c2oc(C(C)C)nc2-c2ccc(F)cc2)n1]

In embodiments, the compound has the formula:

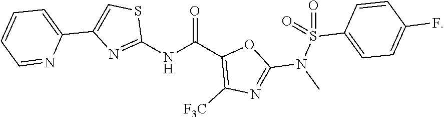 [SMILES: CN(c1nc(C(F)(F)F)c(C(=O)Nc2nc(-c3ccccn3)cs2)o1)S(=O)(=O)c1ccc(F)cc1]

In embodiments, the compound has the formula:

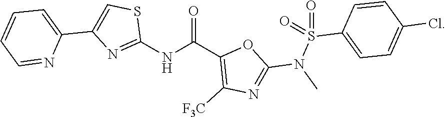 [SMILES: CN(c1nc(C(F)(F)F)c(C(=O)Nc2nc(-c3ccccn3)cs2)o1)S(=O)(=O)c1ccc(Cl)cc1]

In embodiments, the compound has the formula:

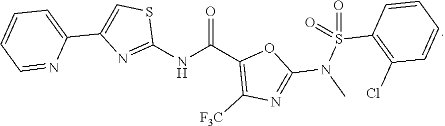 [SMILES: CN(c1nc(C(F)(F)F)c(C(=O)Nc2nc(-c3ccccn3)cs2)o1)S(=O)(=O)c1ccccc1Cl]

In embodiments, the compound has the formula:

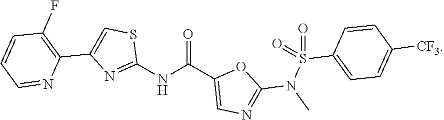 [SMILES: CN(c1ncc(C(=O)Nc2nc(-c3ncccc3F)cs2)o1)S(=O)(=O)c1ccc(C(F)(F)F)cc1]

In embodiments, the compound has the formula:

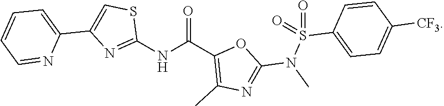 [SMILES: Cc1nc(N(C)S(=O)(=O)c2ccc(C(F)(F)F)cc2)oc1C(=O)Nc1nc(-c2ccccn2)cs1]

In embodiments, the compound has the formula:

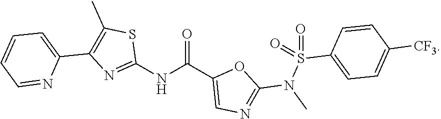 [SMILES: Cc1sc(NC(=O)c2cnc(N(C)S(=O)(=O)c3ccc(C(F)(F)F)cc3)o2)nc1-c1ccccn1]

In embodiments, the compound has the formula:

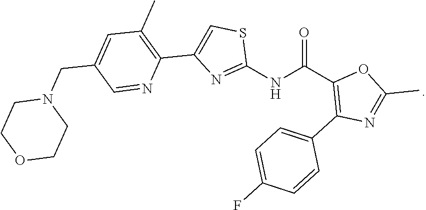 [SMILES: Cc1nc(-c2ccc(F)cc2)c(C(=O)Nc2nc(-c3ncc(CN4CCOCC4)cc3C)cs2)o1]

In embodiments, the compound has the formula:

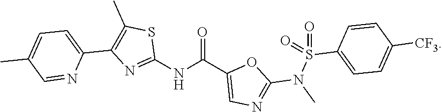 [SMILES: Cc1ccc(-c2nc(NC(=O)c3cnc(N(C)S(=O)(=O)c4ccc(C(F)(F)F)cc4)o3)sc2C)nc1]

In embodiments, the compound has the formula:

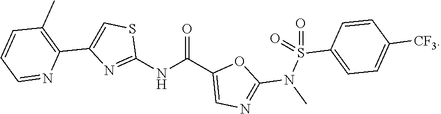 [SMILES: Cc1cccnc1-c1csc(NC(=O)c2cnc(N(C)S(=O)(=O)c3ccc(C(F)(F)F)cc3)o2)n1]

In embodiments, the compound has the formula:

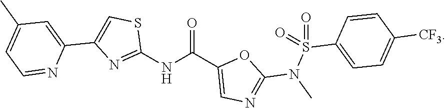 [SMILES: Cc1ccnc(-c2csc(NC(=O)c3cnc(N(C)S(=O)(=O)c4ccc(C(F)(F)F)cc4)o3)n2)c1]

In embodiments, the compound has the formula:

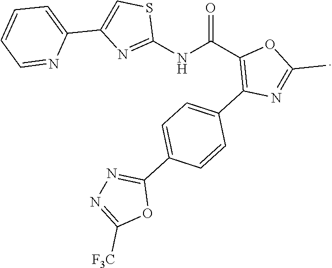 [SMILES: Cc1nc(-c2ccc(-c3nnc(C(F)(F)F)o3)cc2)c(C(=O)Nc2nc(-c3ccccn3)cs2)o1]

In embodiments, the compound has the formula:

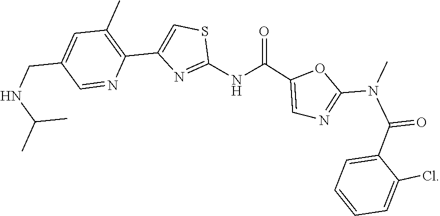 [SMILES: Cc1cc(CNC(C)C)cnc1-c1csc(NC(=O)c2cnc(N(C)C(=O)c3ccccc3Cl)o2)n1]

In embodiments, the compound has the formula:

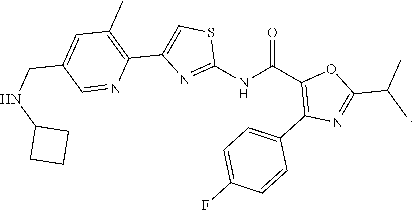 [SMILES: Cc1cc(CNC2CCC2)cnc1-c1csc(NC(=O)c2oc(C(C)C)nc2-c2ccc(F)cc2)n1]

In embodiments, the compound has the formula:

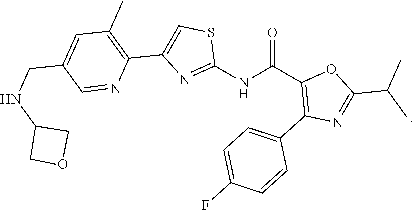 [SMILES: Cc1cc(CNC2COC2)cnc1-c1csc(NC(=O)c2oc(C(C)C)nc2-c2ccc(F)cc2)n1]

In embodiments, the compound has the formula:

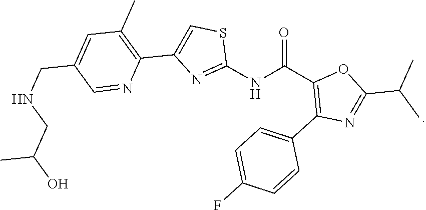 [SMILES: Cc1cc(CNCC(C)O)cnc1-c1csc(NC(=O)c2oc(C(C)C)nc2-c2ccc(F)cc2)n1]

In embodiments, the compound has the formula:

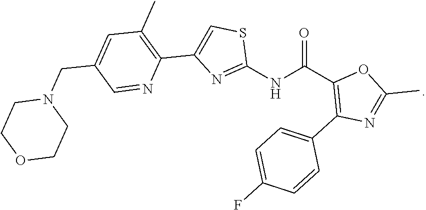 [SMILES: Cc1nc(-c2ccc(F)cc2)c(C(=O)Nc2nc(-c3ncc(CN4CCOCC4)cc3C)cs2)o1]

In embodiments, the compound has the formula:

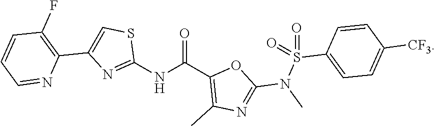 [SMILES: Cc1nc(N(C)S(=O)(=O)c2ccc(C(F)(F)F)cc2)oc1C(=O)Nc1nc(-c2ncccc2F)cs1]

In embodiments, the compound has the formula:

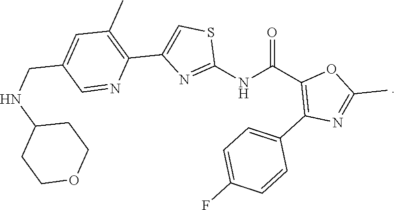 [SMILES: Cc1nc(-c2ccc(F)cc2)c(C(=O)Nc2nc(-c3ncc(CNC4CCOCC4)cc3C)cs2)o1]

In embodiments, the compound has the formula:

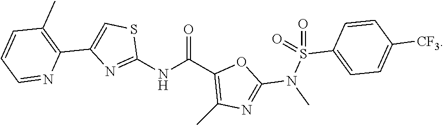 [SMILES: Cc1cccnc1-c1csc(NC(=O)c2oc(N(C)S(=O)(=O)c3ccc(C(F)(F)F)cc3)nc2C)n1]

In embodiments, the compound has the formula:

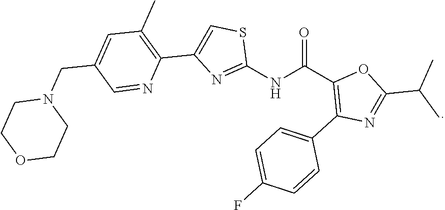 [SMILES: Cc1cc(CN2CCOCC2)cnc1-c1csc(NC(=O)c2oc(C(C)C)nc2-c2ccc(F)cc2)n1]

In embodiments, the compound has the formula:

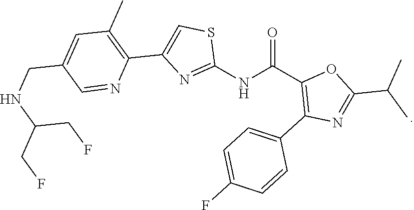 [SMILES: Cc1cc(CNC(CF)CF)cnc1-c1csc(NC(=O)c2oc(C(C)C)nc2-c2ccc(F)cc2)n1]

In embodiments, the compound has the formula

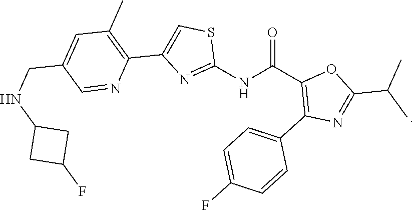 [SMILES: Cc1cc(CNC2CC(F)C2)cnc1-c1csc(NC(=O)c2oc(C(C)C)nc2-c2ccc(F)cc2)n1]

In embodiments, the compound has the formula:

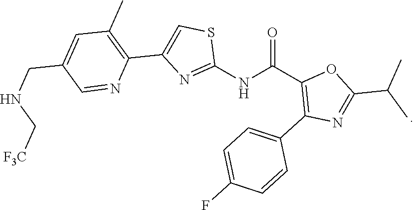 [SMILES: Cc1cc(CNCC(F)(F)F)cnc1-c1csc(NC(=O)c2oc(C(C)C)nc2-c2ccc(F)cc2)n1]

In embodiments, the compound has the formula:

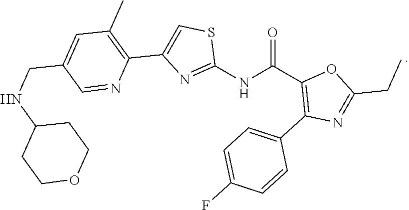 [SMILES: CCc1nc(-c2ccc(F)cc2)c(C(=O)Nc2nc(-c3ncc(CNC4CCOCC4)cc3C)cs2)o1]

In embodiments, the compound has the formula:

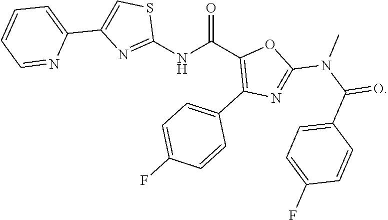 [SMILES: CN(C(=O)c1ccc(F)cc1)c1nc(-c2ccc(F)cc2)c(C(=O)Nc2nc(-c3ccccn3)cs2)o1]

In embodiments, the compound has the formula:

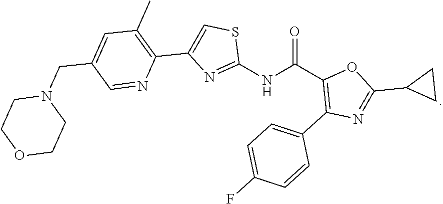 [SMILES: Cc1cc(CN2CCOCC2)cnc1-c1csc(NC(=O)c2oc(C3CC3)nc2-c2ccc(F)cc2)n1]

In embodiments, the compound has the formula

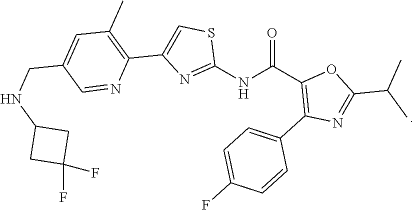 [SMILES: Cc1cc(CNC2CC(F)(F)C2)cnc1-c1csc(NC(=O)c2oc(C(C)C)nc2-c2ccc(F)cc2)n1]

In embodiments, the compound has the formula:

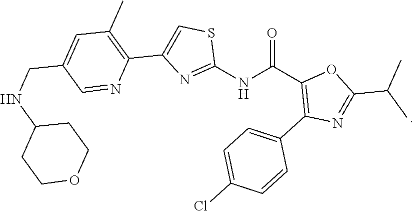 [SMILES: Cc1cc(CNC2CCOCC2)cnc1-c1csc(NC(=O)c2oc(C(C)C)nc2-c2ccc(Cl)cc2)n1]

In embodiments, the compound has the formula:

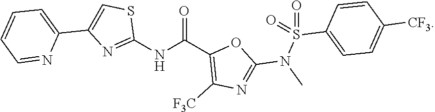 [SMILES: CN(c1nc(C(F)(F)F)c(C(=O)Nc2nc(-c3ccccn3)cs2)o1)S(=O)(=O)c1ccc(C(F)(F)F)cc1]

In embodiments, the compound has the formula:

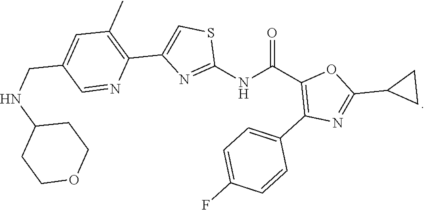 [SMILES: Cc1cc(CNC2CCOCC2)cnc1-c1csc(NC(=O)c2oc(C3CC3)nc2-c2ccc(F)cc2)n1]

In embodiments, the compound has the compound has the formula.

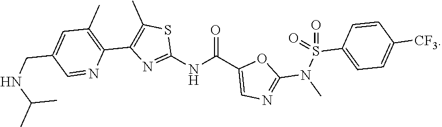 [SMILES: Cc1cc(CNC(C)C)cnc1-c1nc(NC(=O)c2cnc(N(C)S(=O)(=O)c3ccc(C(F)(F)F)cc3)o2)sc1C]

In embodiments, the compound has the formula:

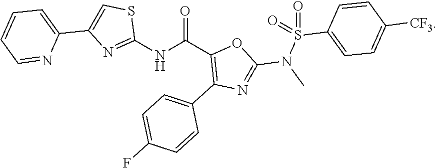 [SMILES: CN(c1nc(-c2ccc(F)cc2)c(C(=O)Nc2nc(-c3ccccn3)cs2)o1)S(=O)(=O)c1ccc(C(F)(F)F)cc1]

In embodiments, the compound has the formula:

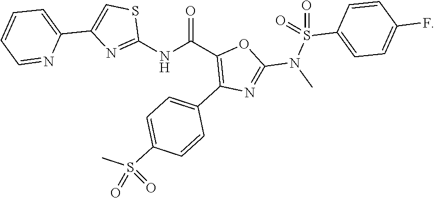 [SMILES: CN(c1nc(-c2ccc(S(C)(=O)=O)cc2)c(C(=O)Nc2nc(-c3ccccn3)cs2)o1)S(=O)(=O)c1ccc(F)cc1]

In embodiments, the compound has the formula:

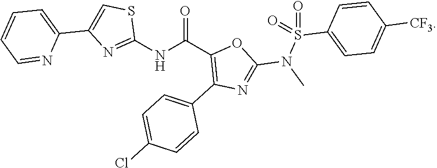 [SMILES: CN(c1nc(-c2ccc(Cl)cc2)c(C(=O)Nc2nc(-c3ccccn3)cs2)o1)S(=O)(=O)c1ccc(C(F)(F)F)cc1]

In embodiments, the compound has the formula:

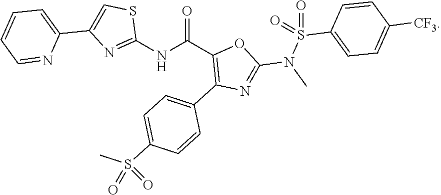 [SMILES: CN(c1nc(-c2ccc(S(C)(=O)=O)cc2)c(C(=O)Nc2nc(-c3ccccn3)cs2)o1)S(=O)(=O)c1ccc(C(F)(F)F)cc1]

In embodiments, the compound has the formula:

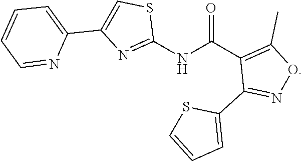 [SMILES: Cc1onc(-c2cccs2)c1C(=O)Nc1nc(-c2ccccn2)cs1]

In embodiments, the compound has the formula:

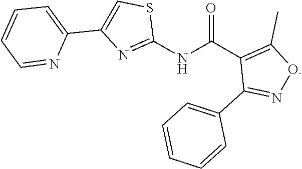 [SMILES: Cc1onc(-c2ccccc2)c1C(=O)Nc1nc(-c2ccccn2)cs1]

In embodiments, the compound has the formula:

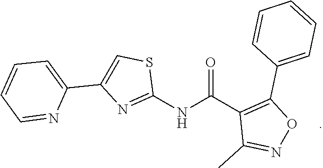 [SMILES: Cc1noc(-c2ccccc2)c1C(=O)Nc1nc(-c2ccccn2)cs1]

In embodiments, the compound has the formula:

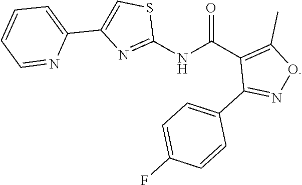 [SMILES: Cc1onc(-c2ccc(F)cc2)c1C(=O)Nc1nc(-c2ccccn2)cs1]

In embodiments, the compound has the formula:

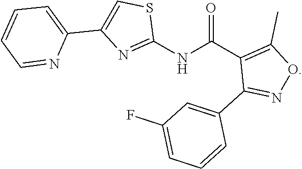 [SMILES: Cc1onc(-c2cccc(F)c2)c1C(=O)Nc1nc(-c2ccccn2)cs1]

In embodiments, the compound has the formula:

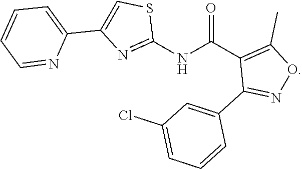 [SMILES: Cc1onc(-c2cccc(Cl)c2)c1C(=O)Nc1nc(-c2ccccn2)cs1]

In embodiments, the compound has the formula:

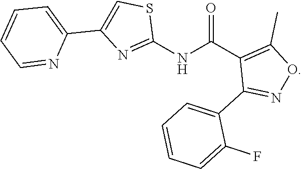 [SMILES: Cc1onc(-c2ccccc2F)c1C(=O)Nc1nc(-c2ccccn2)cs1]

In embodiments, the compound has the formula:

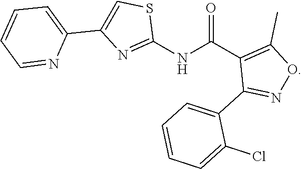 [SMILES: Cc1onc(-c2ccccc2Cl)c1C(=O)Nc1nc(-c2ccccn2)cs1]

In embodiments, the compound has the formula:

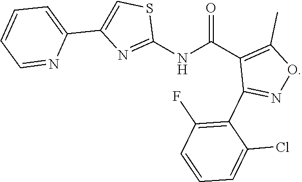 [SMILES: Cc1onc(-c2c(F)cccc2Cl)c1C(=O)Nc1nc(-c2ccccn2)cs1]

In embodiments, the compound has the formula.

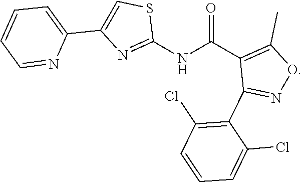 [SMILES: Cc1onc(-c2c(Cl)cccc2Cl)c1C(=O)Nc1nc(-c2ccccn2)cs1]

In embodiments, the compound has the formula:

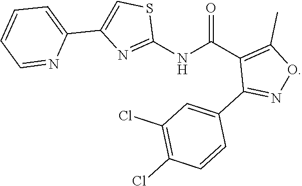 [SMILES: Cc1onc(-c2ccc(Cl)c(Cl)c2)c1C(=O)Nc1nc(-c2ccccn2)cs1]

In embodiments, the compound has the formula:

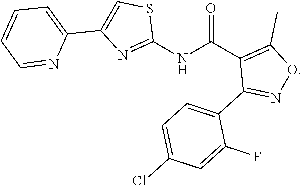 [SMILES: Cc1onc(-c2ccc(Cl)cc2F)c1C(=O)Nc1nc(-c2ccccn2)cs1]

In embodiments, the compound has the formula:

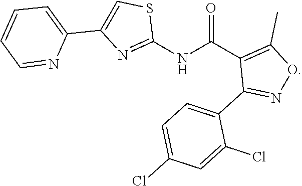 [SMILES: Cc1onc(-c2ccc(Cl)cc2Cl)c1C(=O)Nc1nc(-c2ccccn2)cs1]

In embodiments, the compound has the formula:

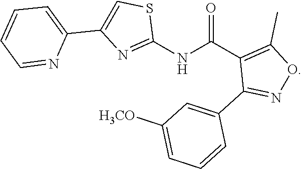 [SMILES: COc1cccc(-c2noc(C)c2C(=O)Nc2nc(-c3ccccn3)cs2)c1]

In embodiments, the compound has the formula:

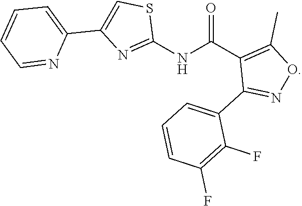 [SMILES: Cc1onc(-c2cccc(F)c2F)c1C(=O)Nc1nc(-c2ccccn2)cs1]

In embodiments, the compound has the formula:

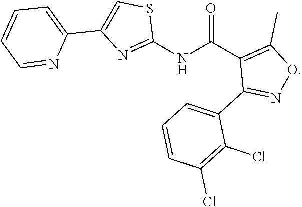 [SMILES: Cc1onc(-c2cccc(Cl)c2Cl)c1C(=O)Nc1nc(-c2ccccn2)cs1]

In embodiments, the compound has the formula:

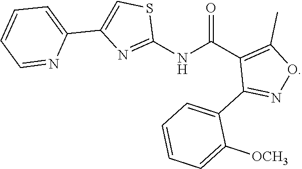 [SMILES: COc1ccccc1-c1noc(C)c1C(=O)Nc1nc(-c2ccccn2)cs1]

In embodiments, the compound has the formula:

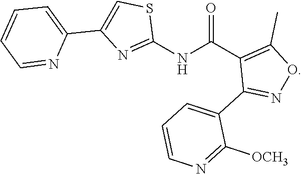 [SMILES: COc1ncccc1-c1noc(C)c1C(=O)Nc1nc(-c2ccccn2)cs1]

In embodiments, the compound has the formula:
In embodiments, the compound has the formula:

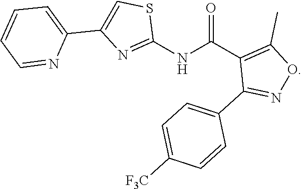 [SMILES: Cc1onc(-c2ccc(C(F)(F)F)cc2)c1C(=O)Nc1nc(-c2ccccn2)cs1]

In embodiments, the compound has the formula:

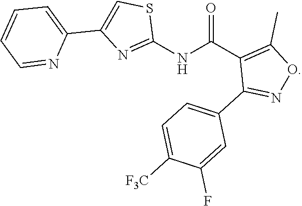 [SMILES: Cc1onc(-c2ccc(C(F)(F)F)c(F)c2)c1C(=O)Nc1nc(-c2ccccn2)cs1]

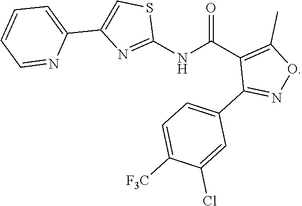 [SMILES: Cc1onc(-c2ccc(C(F)(F)F)c(Cl)c2)c1C(=O)Nc1nc(-c2ccccn2)cs1]

In embodiments, the compound has the formula:

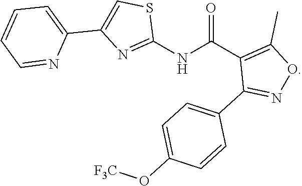 [SMILES: Cc1onc(-c2ccc(OC(F)(F)F)cc2)c1C(=O)Nc1nc(-c2ccccn2)cs1]

In embodiments, the compound has the formula:

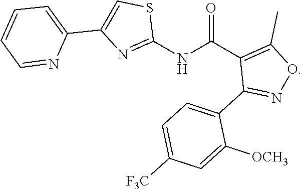 [SMILES: COc1cc(C(F)(F)F)ccc1-c1noc(C)c1C(=O)Nc1nc(-c2ccccn2)cs1]

In embodiments, the compound has the formula:

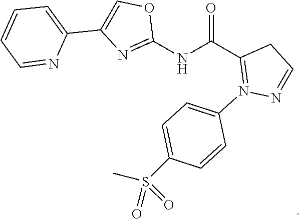

In embodiments, the compound has the formula:

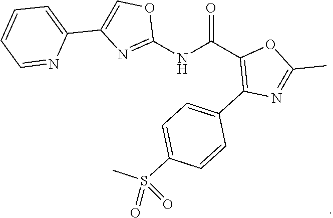 [SMILES: Cc1nc(-c2ccc(S(C)(=O)=O)cc2)c(C(=O)Nc2nc(-c3ccccn3)co2)o1]

In embodiments, the compound has the formula:

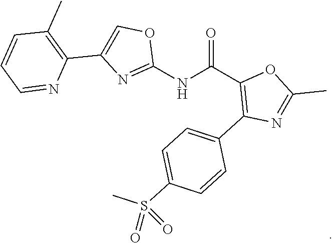 [SMILES: Cc1nc(-c2ccc(S(C)(=O)=O)cc2)c(C(=O)Nc2nc(-c3ncccc3C)co2)o1]

In embodiments, the compound has the formula:

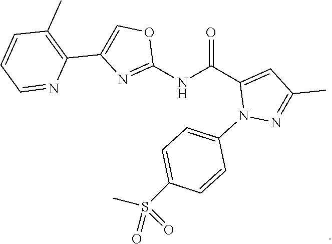 [SMILES: Cc1cc(C(=O)Nc2nc(-c3ncccc3C)co2)n(-c2ccc(S(C)(=O)=O)cc2)n1]

In embodiments, the compound has the formula:

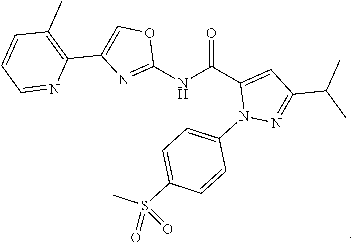 [SMILES: Cc1cccnc1-c1coc(NC(=O)c2cc(C(C)C)nn2-c2ccc(S(C)(=O)=O)cc2)n1]

In embodiments, the compound has the formula:

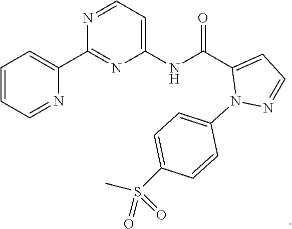 [SMILES: CS(=O)(=O)c1ccc(-n2nccc2C(=O)Nc2ccnc(-c3ccccn3)n2)cc1]

In embodiments, the compound has the formula:

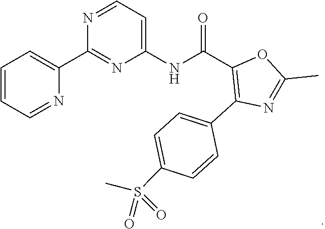 [SMILES: Cc1nc(-c2ccc(S(C)(=O)=O)cc2)c(C(=O)Nc2ccnc(-c3ccccn3)n2)o1]

In embodiments, the compound has the formula:

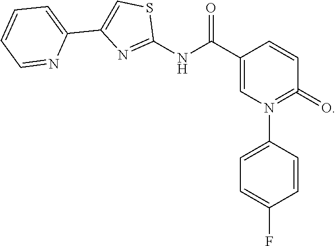

In embodiments, the compound has the formula:

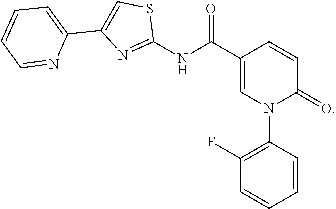 [SMILES: O=C(Nc1nc(-c2ccccn2)cs1)c1ccc(=O)n(-c2ccccc2F)c1]

In embodiments, the compound has the formula:

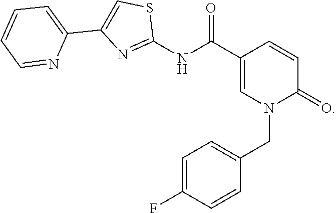 [SMILES: O=C(Nc1nc(-c2ccccn2)cs1)c1ccc(=O)n(Cc2ccc(F)cc2)c1]

embodiments, the compound has the formula:

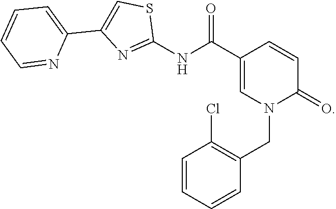 [SMILES: O=C(Nc1nc(-c2ccccn2)cs1)c1ccc(=O)n(Cc2ccccc2Cl)c1]

In embodiments, the compound has the formula:

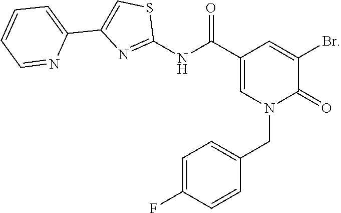 [SMILES: O=C(Nc1nc(-c2ccccn2)cs1)c1cc(Br)c(=O)n(Cc2ccc(F)cc2)c1]

In embodiments, the compound has the formula:

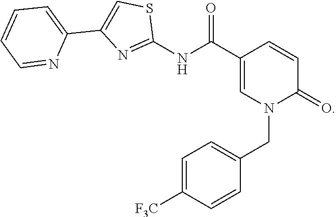 [SMILES: O=C(Nc1nc(-c2ccccn2)cs1)c1ccc(=O)n(Cc2ccc(C(F)(F)F)cc2)c1]

In embodiments, the compound has the formula:

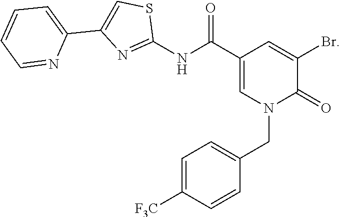 [SMILES: O=C(Nc1nc(-c2ccccn2)cs1)c1cc(Br)c(=O)n(Cc2ccc(C(F)(F)F)cc2)c1]

In embodiments, the compound has the formula:

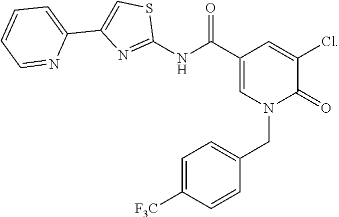 [SMILES: O=C(Nc1nc(-c2ccccn2)cs1)c1cc(Cl)c(=O)n(Cc2ccc(C(F)(F)F)cc2)c1]

In embodiments, the compound has the formula:

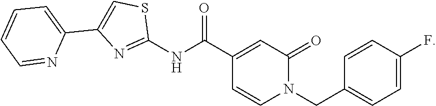 [SMILES: O=C(Nc1nc(-c2ccccn2)cs1)c1ccn(Cc2ccc(F)cc2)c(=O)c1]

In embodiments, the compound has the formula:

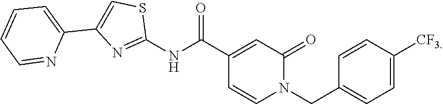 [SMILES: O=C(Nc1nc(-c2ccccn2)cs1)c1ccn(Cc2ccc(C(F)(F)F)cc2)c(=O)c1]

In embodiments, the compound has the formula:

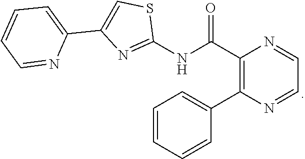 [SMILES: O=C(Nc1nc(-c2ccccn2)cs1)c1nccnc1-c1ccccc1]

In embodiments, the compound has the formula:

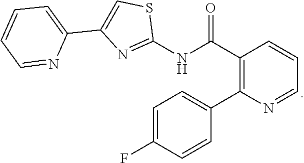 [SMILES: O=C(Nc1nc(-c2ccccn2)cs1)c1cccnc1-c1ccc(F)cc1]

In embodiments, the compound has the formula:

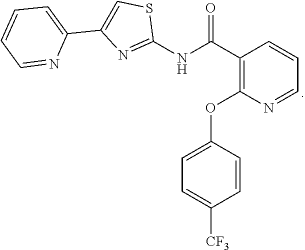 [SMILES: O=C(Nc1nc(-c2ccccn2)cs1)c1cccnc1Oc1ccc(C(F)(F)F)cc1]

In embodiments, the compound has the formula:

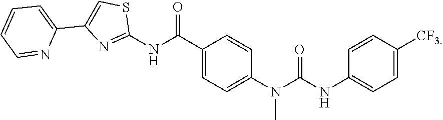 [SMILES: CN(C(=O)Nc1ccc(C(F)(F)F)cc1)c1ccc(C(=O)Nc2nc(-c3ccccn3)cs2)cc1]

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims). In embodiments, the compound is a compound as described in Table 2, Table 5, or Table 8.

In embodiments, the compound is not:

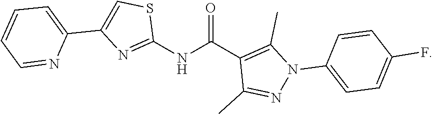 [SMILES: Cc1nn(-c2ccc(F)cc2)c(C)c1C(=O)Nc1nc(-c2ccccn2)cs1]

In embodiments, the compound is not:

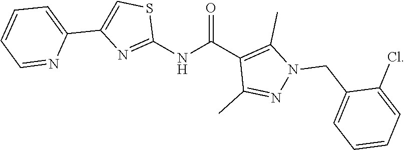 [SMILES: Cc1nn(Cc2ccccc2Cl)c(C)c1C(=O)Nc1nc(-c2ccccn2)cs1]

In embodiments, the compound is not:

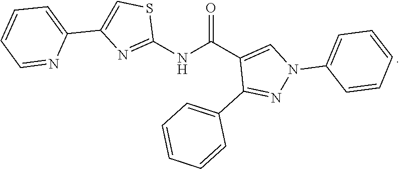 [SMILES: O=C(Nc1nc(-c2ccccn2)cs1)c1cn(-c2ccccc2)nc1-c1ccccc1]

In embodiments, the compound is not:

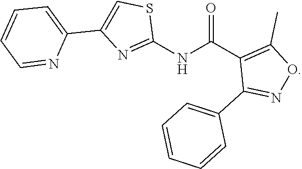 [SMILES: Cc1onc(-c2ccccc2)c1C(=O)Nc1nc(-c2ccccn2)cs1]

In embodiments, the compound is not:

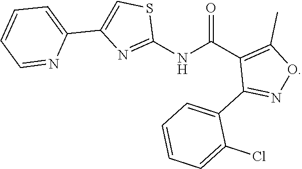 [SMILES: Cc1onc(-c2ccccc2Cl)c1C(=O)Nc1nc(-c2ccccn2)cs1]

In embodiments, the compound is not:

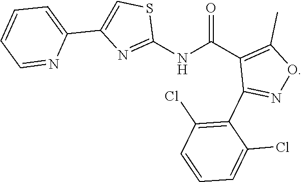 [SMILES: Cc1onc(-c2c(Cl)cccc2Cl)c1C(=O)Nc1nc(-c2ccccn2)cs1]

### III. Pharmaceutical Compositions

In an aspect is provided a pharmaceutical composition including a compound described herein, or a pharmaceutically acceptable salt or tautomer thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the compound is a compound of formula (I), (Ia), (Ib), (Ic), (Id), (Ie), (If), (Ig), (II), (IIa), (III), (IIIa), (IIIb-1), (IIIb), (IIIc-1), (IIIc), (IIId-1), or (IIId), including all embodiments thereof. In embodiments, the compound is a compound of formula (IV), (IVa), (V), (Va), (VI), (VIa), (VII), or (VIIa), including all embodiments thereof. In embodiments, the compound is a compound of formula (VIII), (VIIIa), (VIIIb-1), (VIIIb), (VIIIc-1), (VIIIc), (VIIId), (VIIIe), (VIIIf), (VIIIg), (VIIIh), (VIIIj), (VIIIk), (IX), (IXa), (IXb-1), (IXb), (IXc-1), (IXc), (IXd), (IXe), (IX), (IXg), (IXh), (IXj), (IXk), (X), (Xa), (Xb-1), (Xb), (Xc-1), (Xc), (Xd-1), (Xd), (Xe-1), (Xe), (Xf-1), or (Xf), including all embodiments thereof.

In therapeutic and/or diagnostic applications, the compounds disclosed herein can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

The compounds disclosed herein are effective over a wide dosage range. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate.

Other pharmaceutically acceptable salts may be found in, for example, Remington: The Science and Practice of Pharmacy (20^{th} ed.) Lippincott, Williams & Wilkins (2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, the compounds may be formulated into liquid or solid dosage forms and administered systemically or locally. The compounds may be delivered, for example, in a timed or sustained low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal, or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the compounds disclosed herein may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a subject (e.g., patient) to be treated.

For nasal or inhalation delivery, the compounds disclosed herein may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Pharmaceutical compositions suitable for use disclosed herein include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores.

Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Dosage forms (e.g., compositions) suitable for internal administration contain from about 1.0 milligram to about 5,000 milligrams of active ingredient per unit. In these pharmaceutical compositions, the active ingredient may be present in an amount of about 0.5 to about 95% by weight based on the total weight of the composition. Another convention for denoting the dosage form is in mg per meter squared (mg/m²) of body surface area (BSA).

Typically, an adult will have approximately 1.75 m² of BSA. Based on the body weight of the patient, the dosage may be administered in one or more doses several times per day or per week.

Multiple dosage units may be required to achieve a therapeutically effective amount. For example, if the dosage form is 1,000 mg, and the patient weighs 40 kg, one tablet or capsule will provide a dose of 25 mg per kg for that patient. It will provide a dose of only 12.5 mg/kg for a 80 kg patient.

By way of general guidance, for humans a dosage of as little as about 1 milligram (mg) per kilogram (kg) of body weight and up to about 10,000 mg per kg of body weight is suitable as a therapeutically effective dose. Preferably, from about 5 mg/kg to about 2,500 mg/kg of body weight is used. Other preferred doses range between 25 mg/kg to about 1,000 mg/kg of body weight. However, a dosage of between about 2 milligrams (mg) per kilogram (kg) of body weight to about 400 mg per kg of body weight is also suitable for treating some cancers.

Intravenously, the most preferred rates of administration can range from about 1 to about 1,000 mg/kg/minute during a constant rate infusion. A pharmaceutical composition of the present invention can be administered in a single daily dose, or the total daily dosage may be administered in divided doses of two, three, or four times daily. The composition is generally given in one or more doses on a daily basis or from one to three times a week.

### IV. Methods of Use

In an aspect is provided a method of treating a cancer in a subject in need thereof, the method including administering to the subject in need thereof a therapeutically effective amount of a compound described herein, or a pharmaceutically acceptable salt or tautomer thereof.

In embodiments, the compound is a compound of formula (I), (Ia), (Ib), (Ic), (Id), (Ie), (If), (Ig), (II), (IIa), (III), (IIIa), (IIIb-1), (IIIb), (IIIc-1), (IIIc), (IIId-1), or (IIId), including all embodiments thereof. In embodiments, the compound is a compound of formula (IV), (IVa), (V), (Va), (VI), (VIa), (VII), or (VIIa), including all embodiments thereof. In embodiments, the compound is a compound of formula (VIII), (VIIIa), (VIIIb-1), (VIIIb), (VIIIc-1), (VIIIc), (VIIId), (VIIIe), (VIIIf), (VIIIg), (VIIIh), (VIIIj), (VIIIk), (IX), (IXa), (IXb-1), (IXb), (IXc-1), (IXc), (IXd), (IXe), (I), (IXg), (IXh), (IXj), (IXk), (X), (Xa), (Xb-1), (Xb), (Xc-1), (Xc), (Xd-1), (Xd), (Xe-1), (Xe), (Xf-1), or (Xf), including all embodiments thereof.

In embodiments, the cancer is brain cancer. In embodiments, the cancer is breast cancer. In embodiments, the cancer is colon cancer. In embodiments, the cancer is esophageal cancer. In embodiments, the cancer is gastric cancer. In embodiments, the cancer is gastrointestinal stromal tumor. In embodiments, the cancer is head and neck cancer. In embodiments, the cancer is liver cancer. In embodiments, the cancer is lung cancer. In embodiments, the cancer is lymphoma. In embodiments, the cancer is melanoma. In embodiments, the cancer is pancreatic cancer. In embodiments, the cancer is prostate cancer. In embodiments, the cancer is rectal cancer. In embodiments, the cancer is soft tissue sarcoma. In embodiments, the cancer is bone cancer. In embodiments, the cancer is leukemia.

In an aspect is provided a method of reducing a Wnt-mediated effect on a cell, the method including contacting the cell with an effective amount of a compound as described herein, or a pharmaceutically acceptable salt or tautomer thereof.

In embodiments, the compound is a compound of formula (I), (Ia), (Ib), (Ic), (Id), (he), (If), (Ig), (II), (IIa), (III), (IIIa), (IIIb-1), (IIIb), (IIIc-1), (IIIc), (IIId-1), or (IIId), including all embodiments thereof.

In embodiments, the Wnt-mediated effect on a cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the Wnt-mediated effect on a cell is reduced by about 1.5-fold relative to a control (e.g., absence of the compound). In embodiments, the Wnt-mediated effect on a cell is reduced by about 2-fold relative to a control (e.g., absence of the compound). In embodiments, the Wnt-mediated effect on a cell is reduced by about 5-fold relative to a control (e.g., absence of the compound). In embodiments, the Wnt-mediated effect on a cell is reduced by about 10-fold relative to a control (e.g., absence of the compound). In embodiments, the Wnt-mediated effect on a cell is reduced by about 25-fold relative to a control (e.g., absence of the compound). In embodiments, the Wnt-mediated effect on a cell is reduced by about 50-fold relative to a control (e.g., absence of the compound). In embodiments, the Wnt-mediated effect on a cell is reduced by about 100-fold relative to a control (e.g., absence of the compound). In embodiments, the Wnt-mediated effect on a cell is reduced by about 250-fold relative to a control (e.g., absence of the compound). In embodiments, the Wnt-mediated effect on a cell is reduced by about 500-fold relative to a control (e.g., absence of the compound). In embodiments, the Wnt-mediated effect on a cell is reduced by about 1000-fold relative to a control (e.g., absence of the compound).

In embodiments, the Wnt-mediated effect on a cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the Wnt-mediated effect on a cell is reduced by at least 1.5-fold relative to a control (e.g., absence of the compound). In embodiments, the Wnt-mediated effect on a cell is reduced by at least 2-fold relative to a control (e.g., absence of the compound). In embodiments, the Wnt-mediated effect on a cell is reduced by at least 5-fold relative to a control (e.g., absence of the compound). In embodiments, the Wnt-mediated effect on a cell is reduced by at least 10-fold relative to a control (e.g., absence of the compound). In embodiments, the Wnt-mediated effect on a cell is reduced by at least 25-fold relative to a control (e.g., absence of the compound). In embodiments, the Wnt-mediated effect on a cell is reduced by at least 50-fold relative to a control (e.g., absence of the compound). In embodiments, the Wnt-mediated effect on a cell is reduced by at least 100-fold relative to a control (e.g., absence of the compound). In embodiments, the Wnt-mediated effect on a cell is reduced by at least 250-fold relative to a control (e.g., absence of the compound). In embodiments, the Wnt-mediated effect on a cell is reduced by at least 500-fold relative to a control (e.g., absence of the compound). In embodiments, the Wnt-mediated effect on a cell is reduced by at least 1000-fold relative to a control (e.g., absence of the compound).

In embodiments, the Wnt-mediated effect is an increase in degradation of Pygopus (relative to the degradation of Pygopus in the absence of the compound). In embodiments, the Wnt-mediated effect is an increase in degradation of non-oncogenic beta-Catenin (relative to the degradation of beta-Catenin in the absence of the compound). In embodiments, the Wnt-mediated effect is a decrease in degradation of Axin (relative to the degradation of Axins in the absence of the compound). In embodiments, the Wnt-mediated effect is a decrease in activity of Myc (relative to the activityof Myc in the absence of the compound). In embodiments, the Wnt-mediated effect is a decrease in activity of CD44 (relative to the activity of CD44 in the absence of the compound). In embodiments, the Wnt-mediated effect is a decrease in activity of Axin2 (relative to the activity of Axin 2 in the absence of the compound). In embodiments, the Wnt-mediated effect is a decrease in activity of Bcl-9 (relative to the activity of Bcl-9 in the absence of the compound). In embodiments, the Wnt-mediated effect is a decrease in activity of cyclin D (relative to the activity of cyclin D in the absence of the compound). These Wnt-mediated effects may be assessed using standard assays known in the art.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Examples for Formulae (I), (II), and (III)

Experimental procedures and characterization data
General Procedure A (amide coupling)

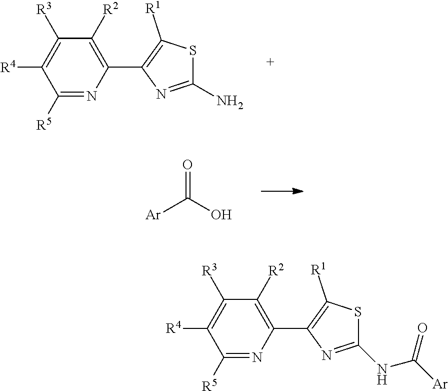

To a stirred solution of an appropriately substituted aromatic carboxylic acid in a suitable organic solvent (e.g., DMF) at room temperature was added 1-2 equivalents of a suitable amide coupling reagent (e.g., HATU) followed by 2-3 equivalents of a suitable organic base (e.g., diisopropylethylamine). A solution of an appropriately substituted aromatic amine in a suitable organic solvent (e.g., DMF) was added to the reaction mixture. The resultant reaction mixture was stirred at room temperature with progress monitored periodically by TLC and/or LCMS. Upon satisfactory conversion of the amine to the desired amide, the reaction mixture was quenched with water and extracted 2-3× with a suitable, water immiscible organic solvent (e.g., EtOAc). The combined organic extracts were washed with aqueous sodium bicarbonate and/or other aqueous media, then dried over a suitable drying agent (e.g., anhydrous MgSO₄). Filtration followed by removal of solvent via rotary evaporation provided a residue that was subsequently purified by normal or reverse phase chromatography to yield the purified amide product.

The following Examples were synthesized using General Procedure A, starting with commercially available or known amine and carboxylic acid building blocks, as shown in Table 1.

TABLE 1

| | | % | m/z | Proton NMR | LHS BB | RHS BB |
|---|---|---|---|---|---|---|
| Example | Structure | Purity | (predicted) | (400 MHz, DMSO-d6) | CAS | CAS |
| 1 | 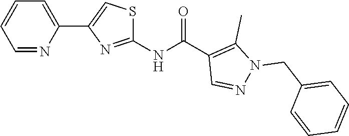 [SMILES: Cc1c(C(=O)Nc2nc(-c3ccccn3)cs2)cnn1Cc1ccccc1] | 99.0 | 376.1 (376.1) | 8.60 (ddd, J = 4.8, 1.8, 0.9 Hz, 1H), 8.42 (d, J = 2.0 Hz, 1H), 8.00 (dt, J = 7.9, 1.1 Hz, 1H), 7.88 (td, J = 7.7, 1.8 Hz, 1H), 7.81 (d, J = 0.9 Hz, 1H), 7.39-7.33 (m, 1H), 7.37-7.29 (m, 2H), | 30235-26-8 | 288252-42-6 |
| | | | | 7.33-7.24 (m, 1H), | | |
| | | | | 7.19-7.12 (m, 2H), | | |
| | | | | 5.39 (s, 2H), 2.55 (s, | | |
| | | | | 3H), NH not observed | | |
| 2 | 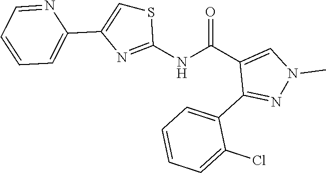 [SMILES: Cn1cc(C(=O)Nc2nc(-c3ccccn3)cs2)c(-c2ccccc2Cl)n1] | 99.4 | 395.9 (396.1) | 12.36 (s, 1H), 8.65 (s, 1H), 8.60 (d, J = 4.8 Hz, 1H), 7.98 (d, J = 7.6 Hz, 1H), 7.90 (t, J = 7.6 Hz, 1H), 7.77 (s, 1H), 7.52 (d, J = 6.8 Hz, 1H), 7.46- 7.37 (m, 3H), 7.34 (t, J = 5.6 Hz, 1H), 3.97 (s, 3H) | 30235-26-8 | 934405-77-3 |
| 3 | 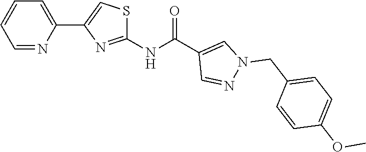 [SMILES: COc1ccc(Cn2cc(C(=O)Nc3nc(-c4ccccn4)cs3)cn2)cc1] | >95 | 392.0 (392.1) | 8.63-8.53 (m, 2H), 8.20 (d, J = 0.7 Hz, 1H), 8.01-7.92 (m, 2H), 7.88 (td, J = 7.7, 1.8 Hz, 1H), 7.82 (d, J = 0.7 Hz, 1H), 7.36- 7.25 (m, 3H), 6.97- 6.89 (m, 2H), 5.31 (s, 2H), 3.74 (s, 3H) | 30235-26-8 | 1105039-93-7 |
| 4 | 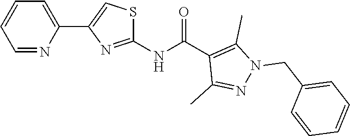 [SMILES: Cc1nn(Cc2ccccc2)c(C)c1C(=O)Nc1nc(-c2ccccn2)cs1] | 95.5 | 390.1 (390.1) | 8.59 (dd, J = 4.6, 1.6 Hz, 1H), 8.00-7.95 (m, 1H), 7.90-7.84 (m, 1H), 7.83 (s, 1H), 7.38-7.25 (m, 4H), 7.20-7.14 (m, 2H), 5.29 (s, 2H), 2.41 (s, 3H), 2.32 (s, 3H), NH not observed | 30235-26-8 | 108444-25-3 |
| 5 | 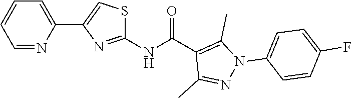 [SMILES: Cc1nn(-c2ccc(F)cc2)c(C)c1C(=O)Nc1nc(-c2ccccn2)cs1] | 98.5 | 394.0 (394.1) | 12.09 (br s, 1H), 8.61 (d, J = 4.80 Hz, 1H), 8.00 (d, J = 7.8 Hz, 1H), 7.87-7.92 (m, 2H), 7.55-7.59 (m, 2H), 7.38-7.43 (m, 2H), 7.32-7.36 (m, | 30235-26-8 | 288251-63-8 |
| | | | | 1H), 2.45 (s, 3H), | | |
| | | | | 2.41 (s, 3H) | | |
| 6 | 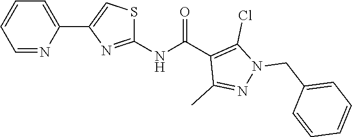 [SMILES: Cc1nn(Cc2ccccc2)c(Cl)c1C(=O)Nc1nc(-c2ccccn2)cs1] | >95 | 410.0 (410.1) | 8.60 (ddd, J = 4.8, 1.8, 0.9 Hz, 1H), 7.96 (dt, J = 7.9, 1.2 Hz, 1H), 7.92-7.83 (m, 1H), 7.87 (s, 1H), 7.42- 7.27 (m, 4H), 7.27- 7.21 (m, 2H), 5.37 (s, 2H), 2.34 (s, 3H), NH not observed | 30235-26-8 | 882227-05-6 |
| 7 | 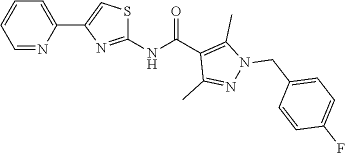 [SMILES: Cc1nn(Cc2ccc(F)cc2)c(C)c1C(=O)Nc1nc(-c2ccccn2)cs1] | >95 | 408.1 (408.1) | 8.60 (ddd, J = 4.8, 1.8, 0.9 Hz, 1H), 7.97 (dt, J = 7.9, 1.1 Hz, 1H), 7.91-7.83 (m, 1H), 7.84 (d, J = 7.4 Hz, 1H), 7.32 (ddd, J = 7.5, 4.8, 1.3 Hz, 1H), 7.28-7.13 (m, 4H), 5.28 (s, 2H), 2.41 (s, 3H), 2.32 (s, | 30235-26-8 | 1154898-82-4 |
| | | | | 3H), NH not observed | | |
| 8 | 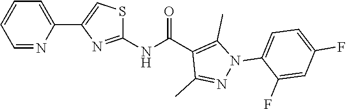 [SMILES: Cc1nn(-c2ccc(F)cc2F)c(C)c1C(=O)Nc1nc(-c2ccccn2)cs1] | 97.3 | 412.1 (412.1) | 12.23 (s, 1H), 8.62 (bs, 1H), 8.00 (d, J = 7.4 Hz, 1H), 7.90-7.86 (m, 2H), 7.69-7.59 (m, 2H), 7.35-7.30 (m, 2H), 2.41 (s, 3H), 2.32 (s, 3H) | 30235-26-8 | 1052558-50-5 |
| 9 | 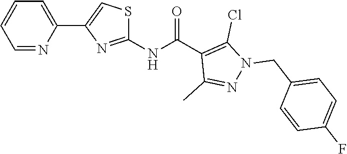 [SMILES: Cc1nn(Cc2ccc(F)cc2)c(Cl)c1C(=O)Nc1nc(-c2ccccn2)cs1] | 99.5 | 428.1 (428.1) | 12.26 (s, 1H), 8.61 (bs, 1H), 7.98 (d, J = 8.0 Hz, 1H), 7.90- 7.86 (m, 2H), 7.35- 7.29 (m, 3H), 7.24 (t, J = 8.4 Hz, 2H), 5.37 (s, 2H), 2.34 (s, 3H) | 30235-26-8 | 956190-97-9 |
| 10 | 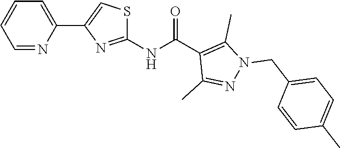 [SMILES: Cc1ccc(Cn2nc(C)c(C(=O)Nc3nc(-c4ccccn4)cs3)c2C)cc1] | >95 | 404.1 (404.1) | 8.60 (ddd, J = 4.8, 1.8, 0.9 Hz, 1H), 8.00-7.93 (m, 1H), 7.91-7.78 (m, 2H), 7.32 (ddd, J = 7.5, 4.7, 1.3 Hz, 1H), 7.15 (d, J = 7.9 Hz, 2H), 7.07 (d, J = 8.0 Hz, 2H), 5.23 (s, 2H), 3.26 (d, J = 16.7 Hz, | 30235-26-8 | 926240-70-2 |
| | | | | 1H), 2.40 (s, 3H), | | |
| | | | | 2.32 (s, 3H), 2.27 (s, | | |
| | | | | 3H), NH not observed | | |
| 11 | 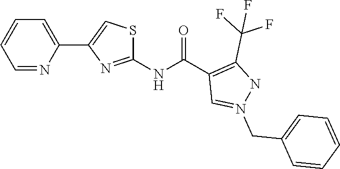 | >95 | 430.1 (430.1) | 8.61 (dt, J = 4.7, 1.4 Hz, 1H), 8.37 (s, 1H), 7.98 (dt, J = 8.0, 1.2 Hz, 1H), 7.93-7.84 (m, 2H), 7.42-7.28 (m, 4H), 7.16 (dd, J = 6.9, 1.9 Hz, 2H), 5.62 (s, 2H), NH not observed | 30235-26-8 | 1946828-44-9 |
| 12 | 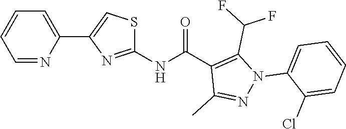 [SMILES: Cc1nn(-c2ccccc2Cl)c(C(F)F)c1C(=O)Nc1nc(-c2ccccn2)cs1] | 99.5 | 446.0 (446.1) | 12.68 (s, 1H), 8.62 (bs, 1H), 8.00 (d, J = 8.0 Hz, 1H), 7.91 (d, J = 8.4 Hz, 1H), 7.91- 7.88 (m, 2H), 7.74 (d, J = 6.0 Hz, 1H), 7.65- 7.58 (m, 3H), 7.36-7.33 (m, 1H), 2.46 (s, 3H) | 30235-26-8 | 137473-93-0 |
| 13 | 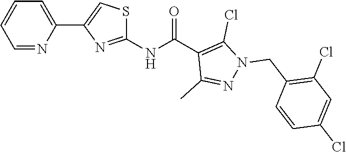 [SMILES: Cc1nn(Cc2ccc(Cl)cc2Cl)c(Cl)c1C(=O)Nc1nc(-c2ccccn2)cs1] | 97.8 | 478.0 (478.0) | 12.23 (s, 1H), 8.62 (bs, 1H), 7.98 (d, J = 8.4 Hz, 1H), 7.90-7.87 (m, 2H), 7.71 (s, 1H), 7.48 (d, J = 8.4 Hz, 1H), 7.35 (t, J = 8.0 Hz, 1H), 7.08 (d, J = 6.0 Hz, 1H), 5.43 (s, 2H), 2.33 (s, 3H) | 30235-26-8 | 956713-96-5 |
| 14 | 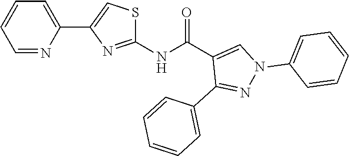 [SMILES: O=C(Nc1nc(-c2ccccn2)cs1)c1cn(-c2ccccc2)nc1-c1ccccc1] | 97.3 | 424.1 (424.1) | 12.55 (s, 1H), 9.38 (s, 1H), 8.62 (bs, 1H), 8.00-7.95 (m, 1H), 7.92-7.82 (m, 6H), 7.63 (t, J = 8.0 Hz, 2H), 7.49-7.44 (m, 4H), 7.36 (t, J = 6.0 Hz, 1H) | 30235-26-8 | 77169-12-1 |
| 15 | 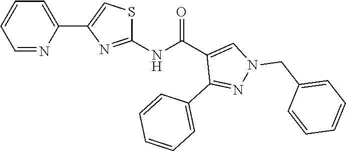 [SMILES: O=C(Nc1nc(-c2ccccn2)cs1)c1cn(Cc2ccccc2)nc1-c1ccccc1] | 99.0 | 452.1 (452.1) | 8.68 (s, 1H), 8.60 (ddd, J = 4.8, 1.8, 1.0 Hz, 1H), 7.96 (dt, J = 7.9, 1.1 Hz, 1H), 7.88 (td, J = 7.7, 1.8 Hz, 1H), 7.81 (s, 1H), 7.72-7.65 (m, 2H), 7.40 (s, 3H), 7.40 (dt, J = 12.5, 1.9 Hz, 3H), 7.39-7.29 (m, 2H), | 30235-26-8 | 905589-98-2 |
| | | | | 5.43 (s, 2H), 3.28 (s, | | |
| | | | | 1H), NH not observed | | |
| 16 | 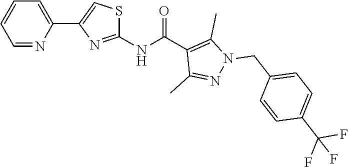 [SMILES: Cc1nn(Cc2ccc(C(F)(F)F)cc2)c(C)c1C(=O)Nc1nc(-c2ccccn2)cs1] | 95.5 | 458.1 (458.1) | 8.60 (ddd, J = 4.8, 1.8, 1.0 Hz, 1H), 7.97 (dt, J = 7.9, 1.1 Hz, 1H), 7.87 (td, J = 7.7, 1.8 Hz, 1H), 7.83 (s, 1H), 7.73 (d, J = 8.1 Hz, 2H), 7.40-7.28 (m, 4H), 5.42 (s, 2H), 2.41 (s, 3H), 2.33 (s, 3H), NH not observed | 30235-26-8 | 1305548-12-2 |
| 17 | 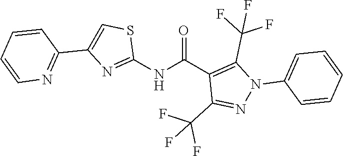 [SMILES: O=C(Nc1nc(-c2ccccn2)cs1)c1c(C(F)(F)F)nn(-c2ccccc2)c1C(F)(F)F] | 98.9 | 484.0 (484.1) | 13.32 (s, 1H), 8.63 (d, J = 4.0 Hz, 1H), 7.98 (s, 1H), 7.88- 7.96 (m, 2H), 7.66- 7.70 (m, 5H), 7.34- 7.38 (m, 1H) | 30235-26-8 | 137216-20-8 |
| 18 | 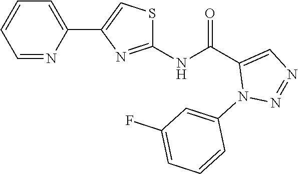 [SMILES: O=C(Nc1nc(-c2ccccn2)cs1)c1cnnn1-c1cccc(F)c1] | 99.2 | 366.9 (367.1) | 13.49 (s, 1H), 8.77 (s, 1H), 8.62 (bs, 1H), 8.01 (d, J = 6.0 Hz, 1H), 7.92-7.88 (m, 2H), 7.65-7.61 (m, 2H), 7.50-7.46 (m, 2H), 7.37 (t, J = 6.8 Hz, 1H) | 30235-26-8 | 1153490-92-6 |
| 19 | 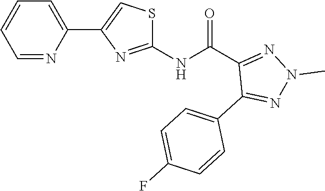 [SMILES: Cn1nc(C(=O)Nc2nc(-c3ccccn3)cs2)c(-c2ccc(F)cc2)n1] | 99.2 | 381.0 (381.1) | 12.86 (s, 1H), 8.61 (bs, 1H), 8.00 (d, J = 7.8 Hz, 1H), 7.94- 7.88 (m, 4H), 7.36- 7.32 (m, 3H), 4.32 (s, 3H) | 30235-26-8 | 483281-63-6 |

The following Examples can be synthesized using General Procedure A, starting with commercially available or known amine and carboxylic acid building blocks. CAS Registry numbers for each amine and carboxylic acid block is indicated in the Table 2.

TABLE 2

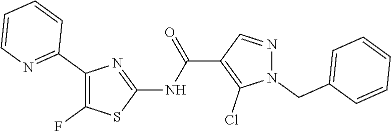 [SMILES: O=C(Nc1nc(-c2ccccn2)c(F)s1)c1cnn(Cc2ccccc2)c1Cl]

Acid CAS 1565755-08-9
Amine CAS 1044270-93-0

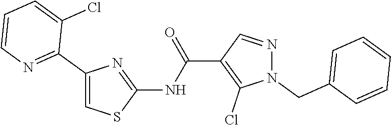 [SMILES: O=C(Nc1nc(-c2ncccc2Cl)cs1)c1cnn(Cc2ccccc2)c1Cl]

Acid CAS 1565755-08-9
Amine CAS 2150665-06-6

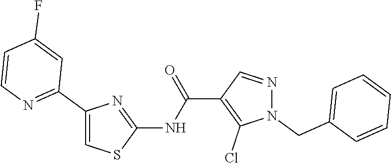 [SMILES: O=C(Nc1nc(-c2cc(F)ccn2)cs1)c1cnn(Cc2ccccc2)c1Cl]

Acid CAS 1565755-08-9
Amine CAS 2228222-84-0

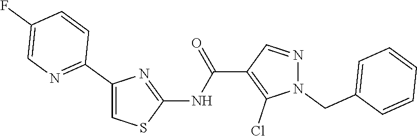 [SMILES: O=C(Nc1nc(-c2ccc(F)cn2)cs1)c1cnn(Cc2ccccc2)c1Cl]

Acid CAS 1565755-08-9
Amine CAS 1551651-30-9

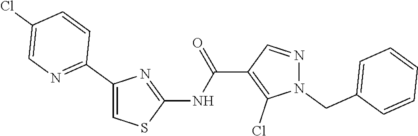 [SMILES: O=C(Nc1nc(-c2ccc(Cl)cn2)cs1)c1cnn(Cc2ccccc2)c1Cl]

Acid CAS 1565755-08-9
Amine CAS 11 [symbol] 5315-17-6

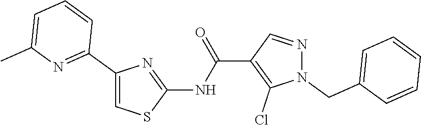 [SMILES: Cc1cccc(-c2csc(NC(=O)c3cnn(Cc4ccccc4)c3Cl)n2)n1]

Acid CAS 1565755-08-9
Amine CAS 99358-99-3

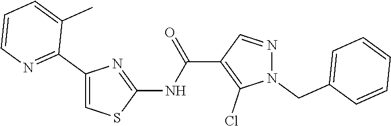 [SMILES: Cc1cccnc1-c1csc(NC(=O)c2cnn(Cc3ccccc3)c2Cl)n1]

Acid CAS 1565755-08-9
Amine CAS 1 [symbol] 44269-91-1

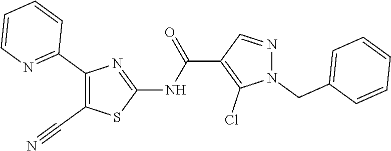 [SMILES: N#Cc1sc(NC(=O)c2cnn(Cc3ccccc3)c2Cl)nc1-c1ccccn1]

Acid CAS 1565755-08-9
Amine CAS 1139743-06-8

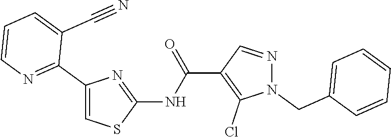 [SMILES: N#Cc1cccnc1-c1csc(NC(=O)c2cnn(Cc3ccccc3)c2Cl)n1]

Acid CAS 1565755-08-9
Amine CAS 2253868-93-6

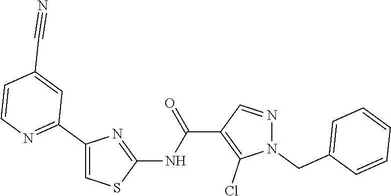 [SMILES: N#Cc1ccnc(-c2csc(NC(=O)c3cnn(Cc4ccccc4)c3Cl)n2)c1]

Acid CAS 1565755-08-9
Amine CAS 2253868-97-0

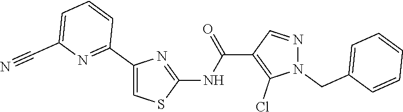 [SMILES: N#Cc1cccc(-c2csc(NC(=O)c3cnn(Cc4ccccc4)c3Cl)n2)n1]

Acid CAS 1565755-08-9
Amine CAS 1185317-90-1

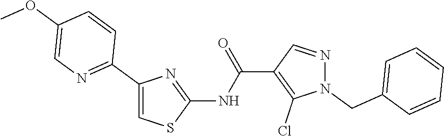 [SMILES: COc1ccc(-c2csc(NC(=O)c3cnn(Cc4ccccc4)c3Cl)n2)nc1]

Acid CAS 1565755-08-9
Amine CAS 2228346-85-6

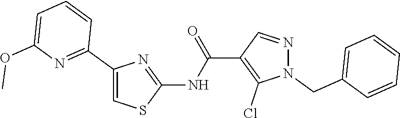 [SMILES: COc1cccc(-c2csc(NC(=O)c3cnn(Cc4ccccc4)c3Cl)n2)n1]

Acid CAS 1565755-08-9
Amine CAS 1 [symbol] 70028-77-5

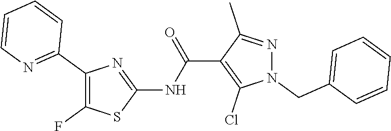 [SMILES: Cc1nn(Cc2ccccc2)c(Cl)c1C(=O)Nc1nc(-c2ccccn2)c(F)s1]

Acid CAS 882227-05-6
Amine CAS 1044270-93-0

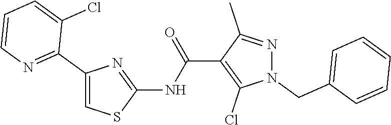 [SMILES: Cc1nn(Cc2ccccc2)c(Cl)c1C(=O)Nc1nc(-c2ncccc2Cl)cs1]

Acid CAS 882227-05-6
Amine CAS 2150665-06-6

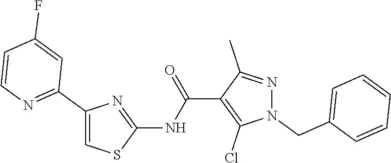 [SMILES: Cc1nn(Cc2ccccc2)c(Cl)c1C(=O)Nc1nc(-c2cc(F)ccn2)cs1]

Acid CAS 882227-05-6
Amine CAS 2228222-84-0

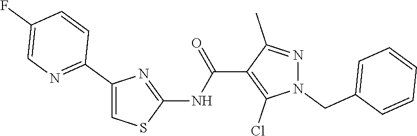 [SMILES: Cc1nn(Cc2ccccc2)c(Cl)c1C(=O)Nc1nc(-c2ccc(F)cn2)cs1]

Acid CAS 882227-05-6
Amine CAS 1551651-30-9

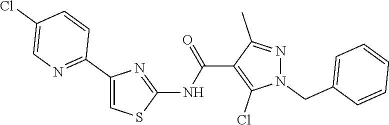 [SMILES: Cc1nn(Cc2ccccc2)c(Cl)c1C(=O)Nc1nc(-c2ccc(Cl)cn2)cs1]

Acid CAS 882227-05-6
Amine CAS 1185315-17-6

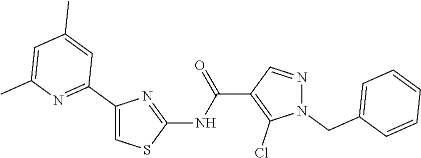 [SMILES: Cc1cc(C)nc(-c2csc(NC(=O)c3cnn(Cc4ccccc4)c3Cl)n2)c1]

Acid CAS 882227-05-6
Amine CAS 1865056-88-7

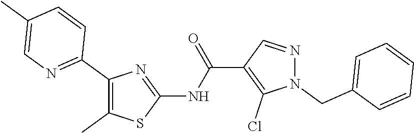 [SMILES: Cc1ccc(-c2nc(NC(=O)c3cnn(Cc4ccccc4)c3Cl)sc2C)nc1]

Acid CAS 882227-05-6
Amine CAS 2294596-48-6

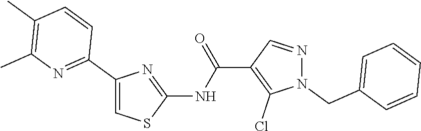 [SMILES: Cc1ccc(-c2csc(NC(=O)c3cnn(Cc4ccccc4)c3Cl)n2)nc1C]

Acid CAS 1565755-08-9
Amine CAS 1934459-93-4

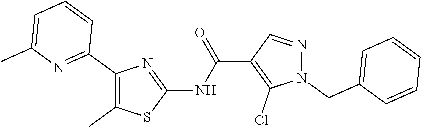 [SMILES: Cc1cccc(-c2nc(NC(=O)c3cnn(Cc4ccccc4)c3Cl)sc2C)n1]

Acid CAS 1565755-08-9
Amine CAS 2169472-77-7

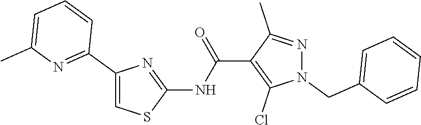 [SMILES: Cc1cccc(-c2csc(NC(=O)c3c(C)nn(Cc4ccccc4)c3Cl)n2)n1]

Acid CAS 882227-05-6
Amine CAS 99358-99-3

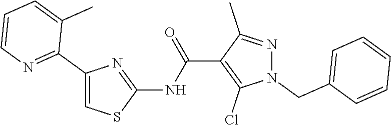 [SMILES: Cc1cccnc1-c1csc(NC(=O)c2c(C)nn(Cc3ccccc3)c2Cl)n1]

Acid CAS 882227-05-6
Amine CAS 1044269-91-1

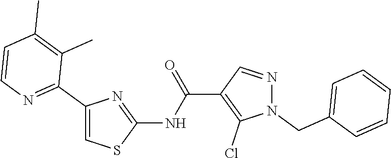 [SMILES: Cc1ccnc(-c2csc(NC(=O)c3cnn(Cc4ccccc4)c3Cl)n2)c1C]

Acid CAS 1565755-08-9
Amine CAS 2169166-95-2

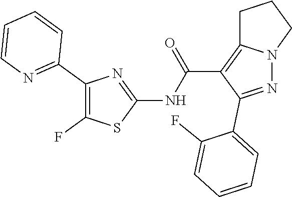 [SMILES: O=C(Nc1nc(-c2ccccn2)c(F)s1)c1c(-c2ccccc2F)nn2c1CCC2]

Acid CAS 1338582-57-2
Amine CAS 1044270-93-0

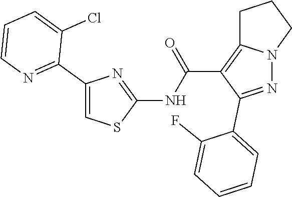 [SMILES: O=C(Nc1nc(-c2ncccc2Cl)cs1)c1c(-c2ccccc2F)nn2c1CCC2]

Acid CAS 1338582-57-2
Amine CAS 21506 [symbol] 5-06-6

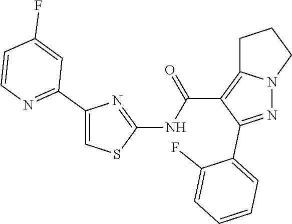 [SMILES: O=C(Nc1nc(-c2cc(F)ccn2)cs1)c1c(-c2ccccc2F)nn2c1CCC2]

Acid CAS 1338582-57-2
Amine CAS 2228222-84-0

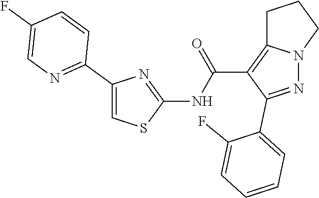 [SMILES: O=C(Nc1nc(-c2ccc(F)cn2)cs1)c1c(-c2ccccc2F)nn2c1CCC2]

Acid CAS 1338582-57-2
Amine CAS 1551651-30-9

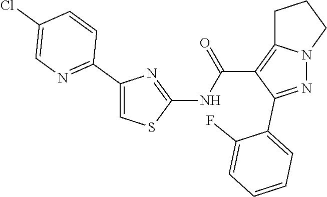 [SMILES: O=C(Nc1nc(-c2ccc(Cl)cn2)cs1)c1c(-c2ccccc2F)nn2c1CCC2]

Acid CAS 1338582-57-2
Amine CAS 1185315-17-6

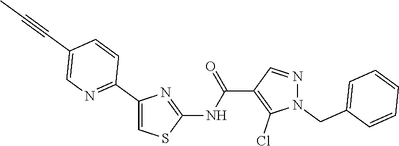 [SMILES: CC#Cc1ccc(-c2csc(NC(=O)c3cnn(Cc4ccccc4)c3Cl)n2)nc1]

Acid CAS 156575 [symbol] -08-9
Amine CAS 2592824-00-3

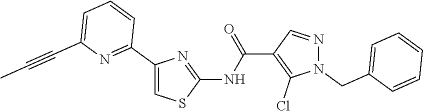 [SMILES: CC#Cc1cccc(-c2csc(NC(=O)c3cnn(Cc4ccccc4)c3Cl)n2)n1]

Acid CAS 1565755-08-9
Amine CAS 2500157-36-4

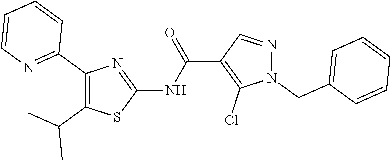 [SMILES: CC(C)c1sc(NC(=O)c2cnn(Cc3ccccc3)c2Cl)nc1-c1ccccn1]

Acid CAS 1565755-08-9
Amine CAS 1225774-92-4

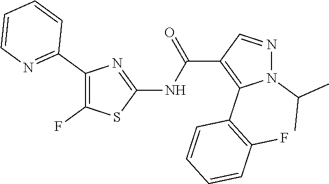 [SMILES: CC(C)n1ncc(C(=O)Nc2nc(-c3ccccn3)c(F)s2)c1-c1ccccc1F]

Acid CAS 17 [symbol] 5583-64-3
Amine CAS 1044270-93-0

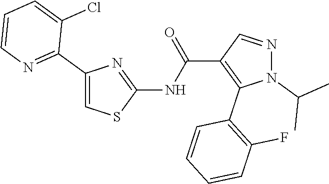 [SMILES: CC(C)n1ncc(C(=O)Nc2nc(-c3ncccc3Cl)cs2)c1-c1ccccc1F]

Acid CAS 1785583-64-z,899
Amine CAS 215 [symbol] 665-06-6

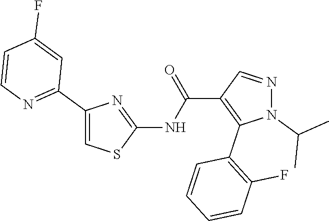 [SMILES: CC(C)n1ncc(C(=O)Nc2nc(-c3cc(F)ccn3)cs2)c1-c1ccccc1F]

Acid CAS 178558 [symbol] -64- [symbol]
Amine CAS 2228222-54-0

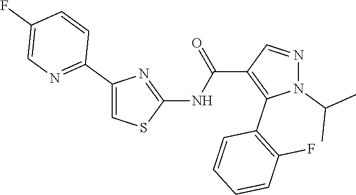 [SMILES: CC(C)n1ncc(C(=O)Nc2nc(-c3ccc(F)cn3)cs2)c1-c1ccccc1F]

Acid CAS 1785583-64-3
Amine CAS 1551651-30-9

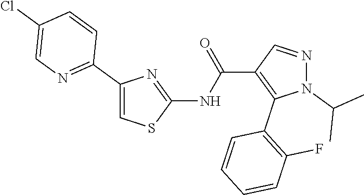 [SMILES: CC(C)n1ncc(C(=O)Nc2nc(-c3ccc(Cl)cn3)cs2)c1-c1ccccc1F]

Acid CAS 1785583-64-3
Amine CAS 1185315-17-6

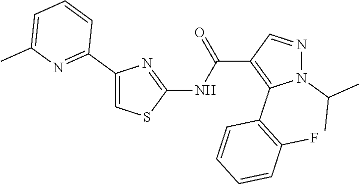 [SMILES: Cc1cccc(-c2csc(NC(=O)c3cnn(C(C)C)c3-c3ccccc3F)n2)n1]

Acid CAS 1785583-64-3
Amine CAS 99358-99-3

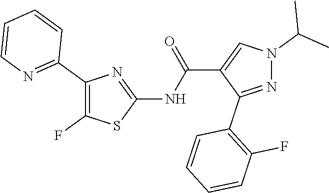 [SMILES: CC(C)n1cc(C(=O)Nc2nc(-c3ccccn3)c(F)s2)c(-c2ccccc2F)n1]

Acid CAS 1306199-51-8
Amine CAS 1044270-93-0

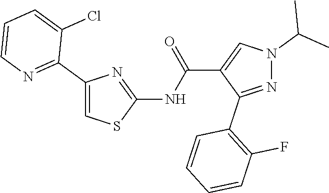 [SMILES: CC(C)n1cc(C(=O)Nc2nc(-c3ncccc3Cl)cs2)c(-c2ccccc2F)n1]

Acid CAS 1306199-51-8
Amine CAS 2150665-06-6

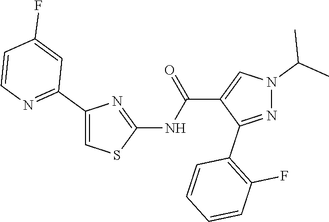 [SMILES: CC(C)n1cc(C(=O)Nc2nc(-c3cc(F)ccn3)cs2)c(-c2ccccc2F)n1]

Acid CAS 1306199-51-8
Amine CAS 2228222-84-0

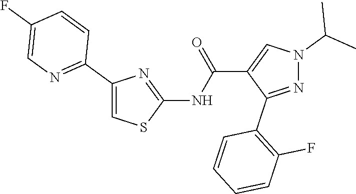 [SMILES: CC(C)n1cc(C(=O)Nc2nc(-c3ccc(F)cn3)cs2)c(-c2ccccc2F)n1]

Acid CAS 1306199-51-8
Amine CAS 1551651-30-9

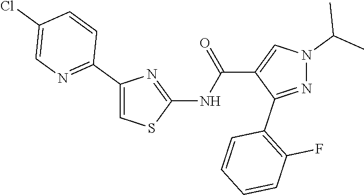 [SMILES: CC(C)n1cc(C(=O)Nc2nc(-c3ccc(Cl)cn3)cs2)c(-c2ccccc2F)n1]

Acid CAS 1306199-51-8
Amine CAS 1185315-17-6

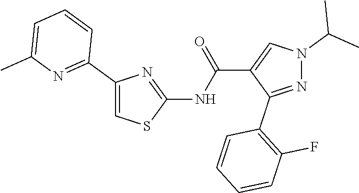 [SMILES: Cc1cccc(-c2csc(NC(=O)c3cn(C(C)C)nc3-c3ccccc3F)n2)n1]

Acid CAS 1306199-51-8
Amine CAS 99358-99-3

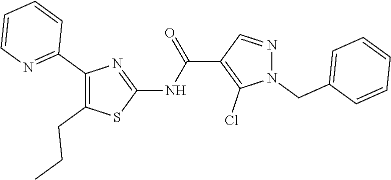 [SMILES: CCCc1sc(NC(=O)c2cnn(Cc3ccccc3)c2Cl)nc1-c1ccccn1]

Acid CAS 1565755-08-9
Amine CAS 2042701-33-5

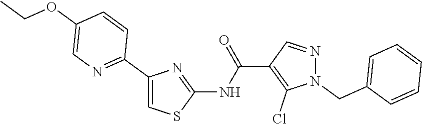 [SMILES: CCOc1ccc(-c2csc(NC(=O)c3cnn(Cc4ccccc4)c3Cl)n2)nc1]

Acid CAS 156575 [symbol] -08-9
Amine CAS 2768587-25-1

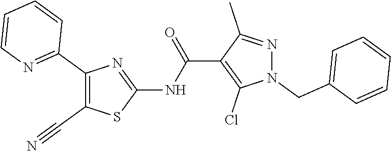 [SMILES: Cc1nn(Cc2ccccc2)c(Cl)c1C(=O)Nc1nc(-c2ccccn2)c(C#N)s1]

Acid CAS 8 [symbol] 2227-05-6
Amine CAS 1139743-06-8

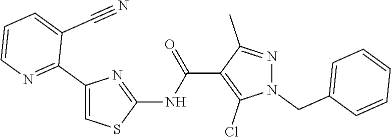 [SMILES: Cc1nn(Cc2ccccc2)c(Cl)c1C(=O)Nc1nc(-c2ncccc2C#N)cs1]

Acid CAS 882227-05-6
Amine CAS 2253868-93-6

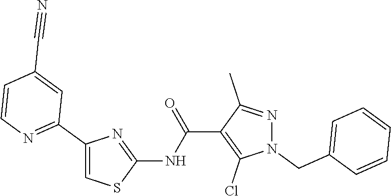 [SMILES: Cc1nn(Cc2ccccc2)c(Cl)c1C(=O)Nc1nc(-c2cc(C#N)ccn2)cs1]

Acid CAS 882227-05-6
Amine CAS 2253868-97-0

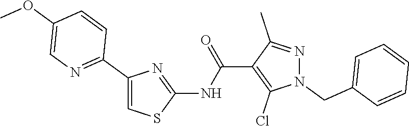 [SMILES: COc1ccc(-c2csc(NC(=O)c3c(C)nn(Cc4ccccc4)c3Cl)n2)nc1]

Acid CAS 882227-05-6
Amine CAS 222 [symbol] 346-85-6

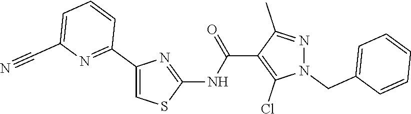 [SMILES: Cc1nn(Cc2ccccc2)c(Cl)c1C(=O)Nc1nc(-c2cccc(C#N)n2)cs1]

Acid CAS 882227-05-6
Amine CAS 11 [symbol] 5317-90-1

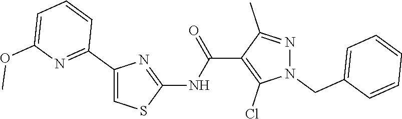 [SMILES: COc1cccc(-c2csc(NC(=O)c3c(C)nn(Cc4ccccc4)c3Cl)n2)n1]

Acid CAS 882227-05-8
Amine CAS 1870028-77-5

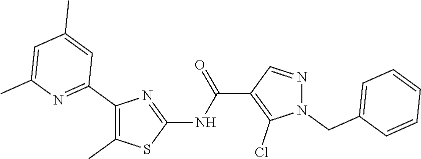 [SMILES: Cc1cc(C)nc(-c2nc(NC(=O)c3cnn(Cc4ccccc4)c3Cl)sc2C)c1]

Acid CAS 156 [symbol] 755-08-9
Amine CAS 2287501-11-3

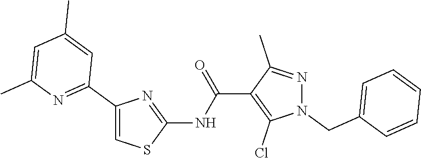 [SMILES: Cc1cc(C)nc(-c2csc(NC(=O)c3c(C)nn(Cc4ccccc4)c3Cl)n2)c1]

Acid CAS 882227-05-6
Amine CAS 1865056- [symbol] 8-7

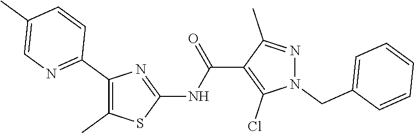 [SMILES: Cc1ccc(-c2nc(NC(=O)c3c(C)nn(Cc4ccccc4)c3Cl)sc2C)nc1]

Acid CAS 882227-05-6
Amine CAS 2294596-48-6

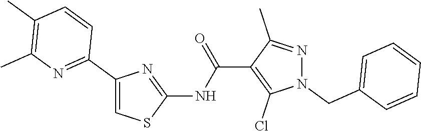 [SMILES: Cc1ccc(-c2csc(NC(=O)c3c(C)nn(Cc4ccccc4)c3Cl)n2)nc1C]

Acid CAS 882227-05-6
Amine CAS 1934459-93-4

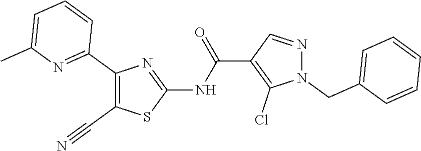 [SMILES: Cc1cccc(-c2nc(NC(=O)c3cnn(Cc4ccccc4)c3Cl)sc2C#N)n1]

Acid CAS 1565755-08-9
Amine CAS 1975266-69-3

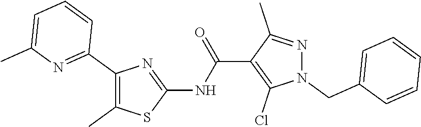 [SMILES: Cc1cccc(-c2nc(NC(=O)c3c(C)nn(Cc4ccccc4)c3Cl)sc2C)n1]

Acid CAS 882227-05-6
Amine CAS 2169472-77-7

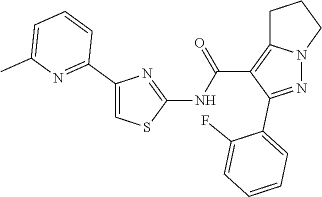 [SMILES: Cc1cccc(-c2csc(NC(=O)c3c(-c4ccccc4F)nn4c3CCC4)n2)n1]

Acid CAS 1338582-57-2
Amine CAS 99358-99-3

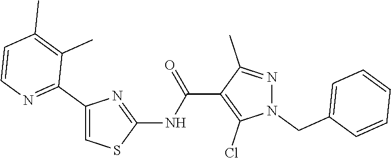 [SMILES: Cc1ccnc(-c2csc(NC(=O)c3c(C)nn(Cc4ccccc4)c3Cl)n2)c1C]

Acid CAS 882227-05-6
Amine CAS 2169166-95-2

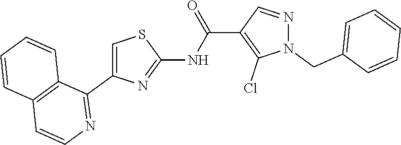 [SMILES: O=C(Nc1nc(-c2nccc3ccccc23)cs1)c1cnn(Cc2ccccc2)c1Cl]

Acid CAS 1565755-08-9
Amine CAS 651042-73-6

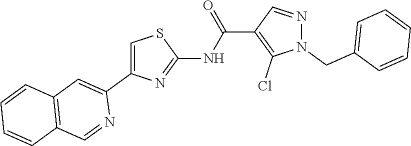 [SMILES: O=C(Nc1nc(-c2cc3ccccc3cn2)cs1)c1cnn(Cc2ccccc2)c1Cl]

Acid CAS 1565755-08-9
Amine CAS 2742868-53-3

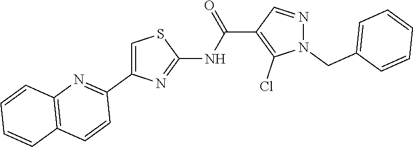 [SMILES: O=C(Nc1nc(-c2ccc3ccccc3n2)cs1)c1cnn(Cc2ccccc2)c1Cl]

Acid CAS 1565755-08-9
Amine CAS 30235-29-1

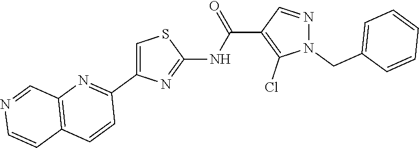 [SMILES: O=C(Nc1nc(-c2ccc3ccncc3n2)cs1)c1cnn(Cc2ccccc2)c1Cl]

Acid CAS 1565755-08-9
Amine CAS 2604301-23-5

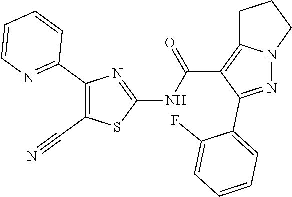 [SMILES: N#Cc1sc(NC(=O)c2c(-c3ccccc3F)nn3c2CCC3)nc1-c1ccccn1]

Acid CAS 1338582-57-2
Amine CAS 1139743-06-8

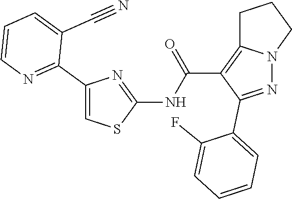 [SMILES: N#Cc1cccnc1-c1csc(NC(=O)c2c(-c3ccccc3F)nn3c2CCC3)n1]

Acid CAS 1338562-57-2
Amine CAS 2253868-93-6

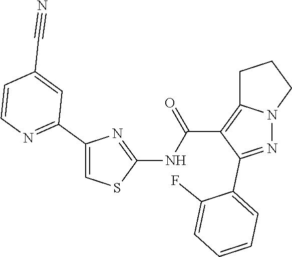 [SMILES: N#Cc1ccnc(-c2csc(NC(=O)c3c(-c4ccccc4F)nn4c3CCC4)n2)c1]

Acid CAS 1338582-57-2
Amine CAS 2253868-97-0

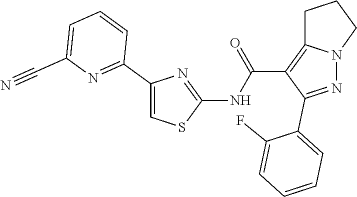 [SMILES: N#Cc1cccc(-c2csc(NC(=O)c3c(-c4ccccc4F)nn4c3CCC4)n2)n1]

Acid CAS 1338582-57-2
Amine CAS 1185317-9 [symbol] 1

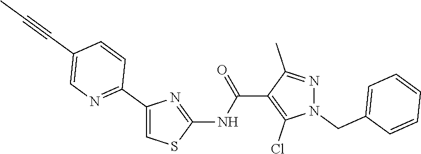 [SMILES: CC#Cc1ccc(-c2csc(NC(=O)c3c(C)nn(Cc4ccccc4)c3Cl)n2)nc1]

Acid CAS 882227-05-6
Amine CAS 2592824-00-3

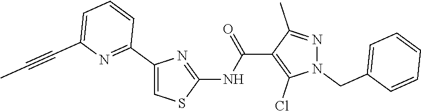 [SMILES: CC#Cc1cccc(-c2csc(NC(=O)c3c(C)nn(Cc4ccccc4)c3Cl)n2)n1]

Acid CAS 882227-05-6
Amine CAS 2600157-36-4

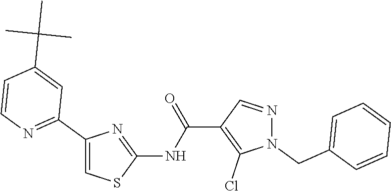 [SMILES: CC(C)(C)c1ccnc(-c2csc(NC(=O)c3cnn(Cc4ccccc4)c3Cl)n2)c1]

Acid CAS 1565755-08-9
Amine CAS 2253868-94-7

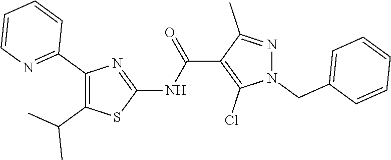 [SMILES: Cc1nn(Cc2ccccc2)c(Cl)c1C(=O)Nc1nc(-c2ccccn2)c(C(C)C)s1]

Acid CAS 882227-05-6
Amine CAS 1225774-92-4

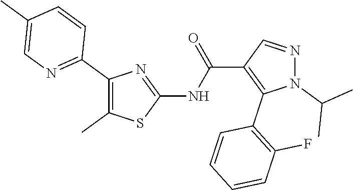 [SMILES: Cc1ccc(-c2nc(NC(=O)c3cnn(C(C)C)c3-c3ccccc3F)sc2C)nc1]

Acid CAS 1785583-64-3
Amine CAS 2294596-48-6

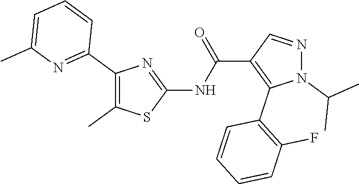 [SMILES: Cc1cccc(-c2nc(NC(=O)c3cnn(C(C)C)c3-c3ccccc3F)sc2C)n1]

Acid CAS 1785583-64-3
Amine CAS 2169472-77-7

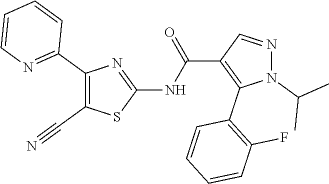 [SMILES: CC(C)n1ncc(C(=O)Nc2nc(-c3ccccn3)c(C#N)s2)c1-c1ccccc1F]

Acid CAS 1785583-64-3
Amine CAS 1139743-06-8

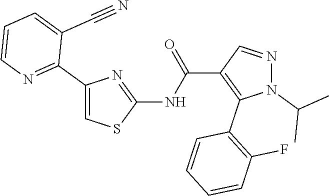 [SMILES: CC(C)n1ncc(C(=O)Nc2nc(-c3ncccc3C#N)cs2)c1-c1ccccc1F]

Acid CAS 1785583-64-3
Amine CAS 2253868-93-6

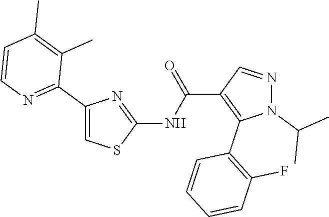 [SMILES: Cc1ccnc(-c2csc(NC(=O)c3cnn(C(C)C)c3-c3ccccc3F)n2)c1C]

Acid CAS 1785583-64-3
Amine CAS 2169166-95-2

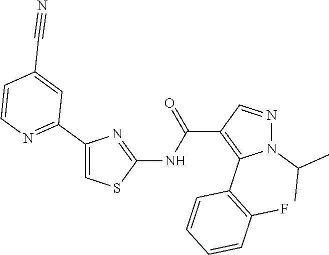 [SMILES: CC(C)n1ncc(C(=O)Nc2nc(-c3cc(C#N)ccn3)cs2)c1-c1ccccc1F]

Acid CAS 1785583-64-3
Amine CAS 2253868-97-0

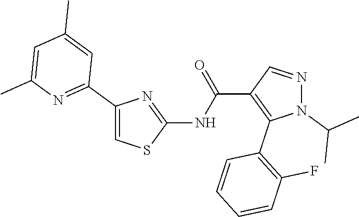 [SMILES: Cc1cc(C)nc(-c2csc(NC(=O)c3cnn(C(C)C)c3-c3ccccc3F)n2)c1]

Acid CAS 1785583-64-3
Amine CAS 1865056-88-7

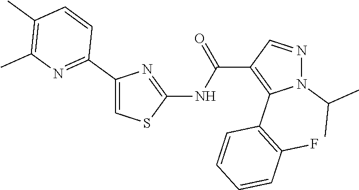 [SMILES: Cc1ccc(-c2csc(NC(=O)c3cnn(C(C)C)c3-c3ccccc3F)n2)nc1C]

Acid CAS 1785583-64-3
Amine CAS 1934459-93-4

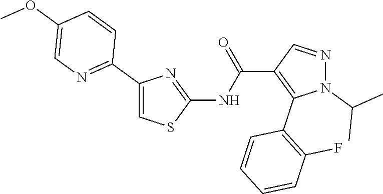 [SMILES: COc1ccc(-c2csc(NC(=O)c3cnn(C(C)C)c3-c3ccccc3F)n2)nc1]

Acid CAS 1785583-64-3
Amine CAS 2228346-85-6

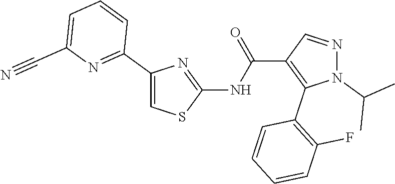 [SMILES: CC(C)n1ncc(C(=O)Nc2nc(-c3cccc(C#N)n3)cs2)c1-c1ccccc1F]

Acid CAS 1785583-64-3
Amine CAS 1185317-90-1

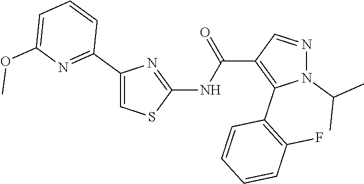 [SMILES: COc1cccc(-c2csc(NC(=O)c3cnn(C(C)C)c3-c3ccccc3F)n2)n1]

Acid CAS 1785583-64-3
Amine CAS 1870028-77-5

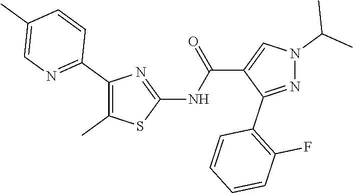 [SMILES: Cc1ccc(-c2nc(NC(=O)c3cn(C(C)C)nc3-c3ccccc3F)sc2C)nc1]

Acid CAS 1306199- [symbol] 1-8
Amine CAS 2294596-48-6

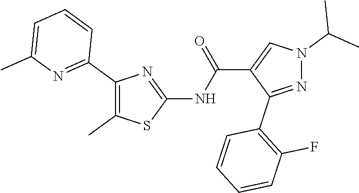 [SMILES: Cc1cccc(-c2nc(NC(=O)c3cn(C(C)C)nc3-c3ccccc3F)sc2C)n1]

Acid CAS 1306199-51-8
Amine CAS 2169472-77-7

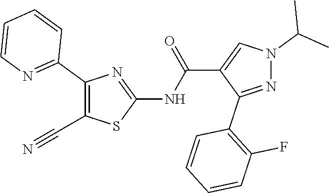 [SMILES: CC(C)n1cc(C(=O)Nc2nc(-c3ccccn3)c(C#N)s2)c(-c2ccccc2F)n1]

Acid CAS 1306199-51-8
Amine CAS 1139743-06-8

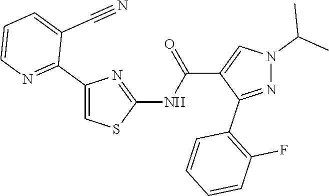 [SMILES: CC(C)n1cc(C(=O)Nc2nc(-c3ncccc3C#N)cs2)c(-c2ccccc2F)n1]

Acid CAS 1306199-51-8
Amine CAS 2253868-93-6

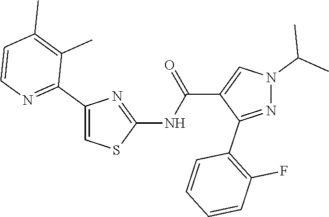 [SMILES: Cc1ccnc(-c2csc(NC(=O)c3cn(C(C)C)nc3-c3ccccc3F)n2)c1C]

Acid CAS 1306199-51-8
Amine CAS 2169166-95-2

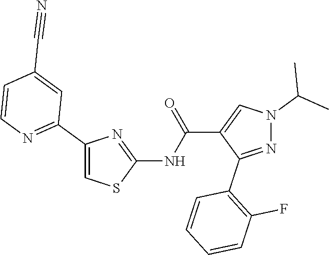 [SMILES: CC(C)n1cc(C(=O)Nc2nc(-c3cc(C#N)ccn3)cs2)c(-c2ccccc2F)n1]

Acid CAS 1306199-51-8
Amine CAS 2253868-97-0

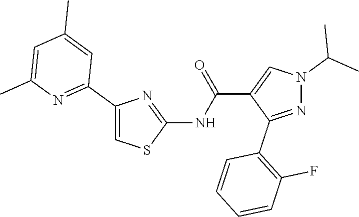 [SMILES: Cc1cc(C)nc(-c2csc(NC(=O)c3cn(C(C)C)nc3-c3ccccc3F)n2)c1]

Acid CAS 1306199-51-8
Amine CAS 1865056-88-7

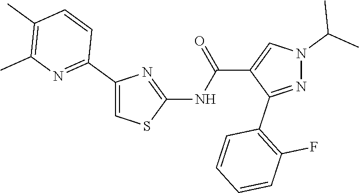 [SMILES: Cc1ccc(-c2csc(NC(=O)c3cn(C(C)C)nc3-c3ccccc3F)n2)nc1C]

Acid CAS 1306199-51-8
Amine CAS 1934459-93-4

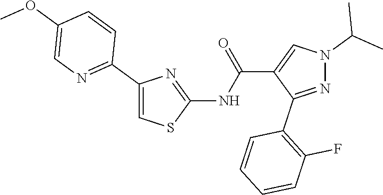 [SMILES: COc1ccc(-c2csc(NC(=O)c3cn(C(C)C)nc3-c3ccccc3F)n2)nc1]

Acid CAS 1306199-51-8
Amine CAS 2228346- [symbol] 5-6

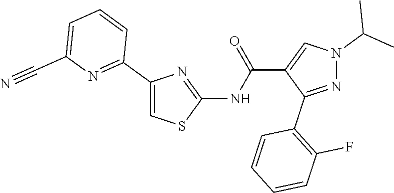 [SMILES: CC(C)n1cc(C(=O)Nc2nc(-c3cccc(C#N)n3)cs2)c(-c2ccccc2F)n1]

Acid CAS 1306199-51-8
Amine CAS 1185317-90-1

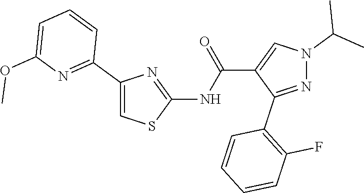 [SMILES: COc1cccc(-c2csc(NC(=O)c3cn(C(C)C)nc3-c3ccccc3F)n2)n1]

Acid CAS 1306199-51-8
Amine CAS 1870028-77-5

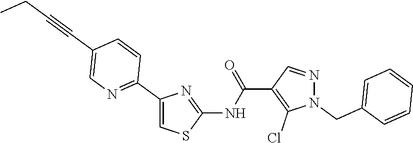 [SMILES: CCC#Cc1ccc(-c2csc(NC(=O)c3cnn(Cc4ccccc4)c3Cl)n2)nc1]

Acid CAS 1565755-08-9
Amine CAS 2538808-37-4

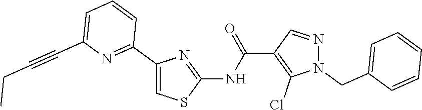 [SMILES: CCC#Cc1cccc(-c2csc(NC(=O)c3cnn(Cc4ccccc4)c3Cl)n2)n1]

Acid CAS 1565755-08-9
Amine CAS 2539204-54-9

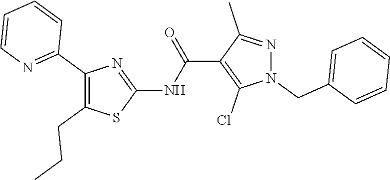 [SMILES: CCCc1sc(NC(=O)c2c(C)nn(Cc3ccccc3)c2Cl)nc1-c1ccccn1]

Acid CAS 882227-05-6
Amine CAS 2042701-33-5

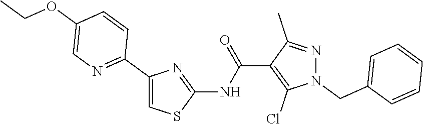 [SMILES: CCOc1ccc(-c2csc(NC(=O)c3c(C)nn(Cc4ccccc4)c3Cl)n2)nc1]

Acid CAS 882227-05-6
Amine CAS 2768587-25-1

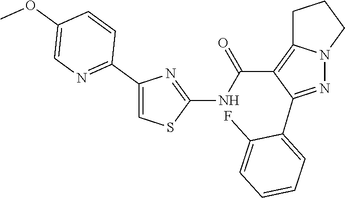 [SMILES: COc1ccc(-c2csc(NC(=O)c3c(-c4ccccc4F)nn4c3CCC4)n2)nc1]

Acid CAS 1338582-57-2
Amine CAS 2228346-85-6

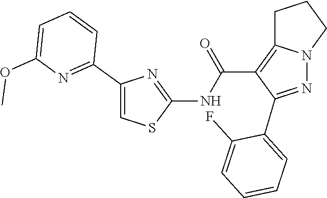 [SMILES: COc1cccc(-c2csc(NC(=O)c3c(-c4ccccc4F)nn4c3CCC4)n2)n1]

Acid CAS 1338582-57-2
Amine CAS 1870028-77-5

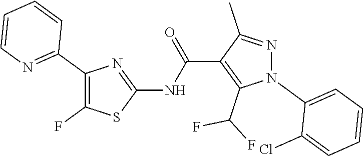 [SMILES: Cc1nn(-c2ccccc2Cl)c(C(F)F)c1C(=O)Nc1nc(-c2ccccn2)c(F)s1]

Acid CAS 1375473-93-0
Amine CAS 1044270-93-0

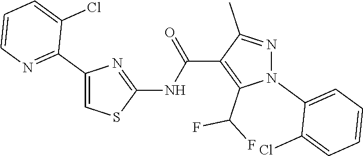 [SMILES: Cc1nn(-c2ccccc2Cl)c(C(F)F)c1C(=O)Nc1nc(-c2ncccc2Cl)cs1]

Acid CAS 1375473-93-0
Amine CAS 2150665-06-6

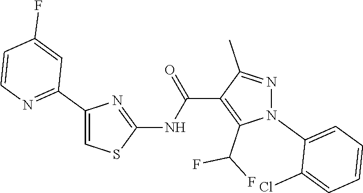 [SMILES: Cc1nn(-c2ccccc2Cl)c(C(F)F)c1C(=O)Nc1nc(-c2cc(F)ccn2)cs1]

Acid CAS 1375473-93-0
Amine CAS 2228222-84-0

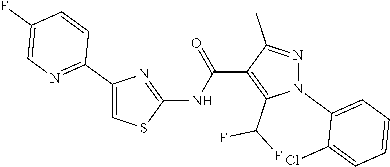 [SMILES: Cc1nn(-c2ccccc2Cl)c(C(F)F)c1C(=O)Nc1nc(-c2ccc(F)cn2)cs1]

Acid CAS 1375473-93-0
Amine CAS 1551651-30-9

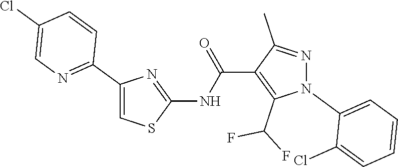 [SMILES: Cc1nn(-c2ccccc2Cl)c(C(F)F)c1C(=O)Nc1nc(-c2ccc(Cl)cn2)cs1]

Acid CAS 1375473-93-0
Amine CAS 1185315-17-6

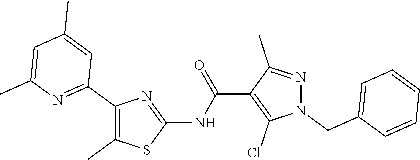 [SMILES: Cc1cc(C)nc(-c2nc(NC(=O)c3c(C)nn(Cc4ccccc4)c3Cl)sc2C)c1]

Acid CAS 882227-05-6
Amine CAS 2287501-11-3

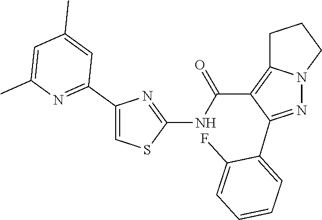 [SMILES: Cc1cc(C)nc(-c2csc(NC(=O)c3c(-c4ccccc4F)nn4c3CCC4)n2)c1]

Acid CAS 1338582-57-2
Amine CAS 1865056-88-7

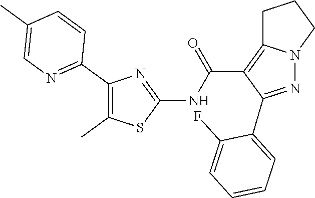 [SMILES: Cc1ccc(-c2nc(NC(=O)c3c(-c4ccccc4F)nn4c3CCC4)sc2C)nc1]

Acid CAS 1338582-57-2
Amine CAS 2294596-48-6

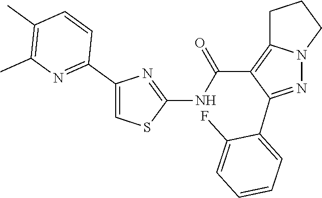 [SMILES: Cc1ccc(-c2csc(NC(=O)c3c(-c4ccccc4F)nn4c3CCC4)n2)nc1C]

Acid CAS 1338582-57-2
Amine CAS 1934459-93-4

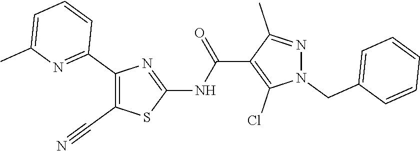 [SMILES: Cc1cccc(-c2nc(NC(=O)c3c(C)nn(Cc4ccccc4)c3Cl)sc2C#N)n1]

Acid CAS 882227-05-6
Amine CAS 1975268-69-3

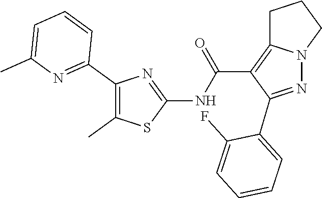 [SMILES: Cc1cccc(-c2nc(NC(=O)c3c(-c4ccccc4F)nn4c3CCC4)sc2C)n1]

Acid CAS 1338582-57-2
Amine CAS 2169472-77-7

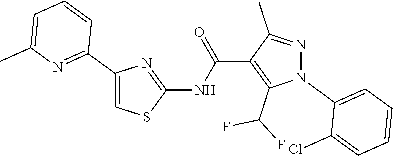 [SMILES: Cc1cccc(-c2csc(NC(=O)c3c(C)nn(-c4ccccc4Cl)c3C(F)F)n2)n1]

Acid CAS 1375473-93-0
Amine CAS 99358-99-3

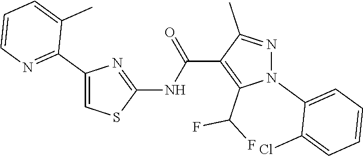 [SMILES: Cc1cccnc1-c1csc(NC(=O)c2c(C)nn(-c3ccccc3Cl)c2C(F)F)n1]

Acid CAS 1375473-93-0
Amine CAS 1044269-91-1

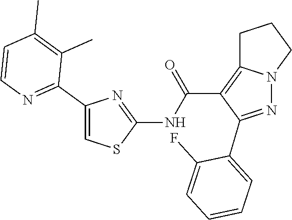 [SMILES: Cc1ccnc(-c2csc(NC(=O)c3c(-c4ccccc4F)nn4c3CCC4)n2)c1C]

Acid CAS 1338582-57-2
Amine CAS 2169166-95-2

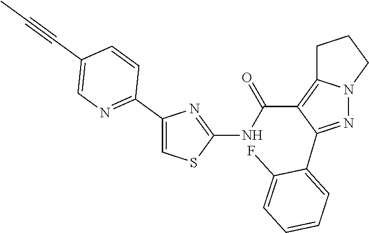 [SMILES: CC#Cc1ccc(-c2csc(NC(=O)c3c(-c4ccccc4F)nn4c3CCC4)n2)nc1]

Acid CAS 1338582-57-2
Amine CAS 2592824-00-3

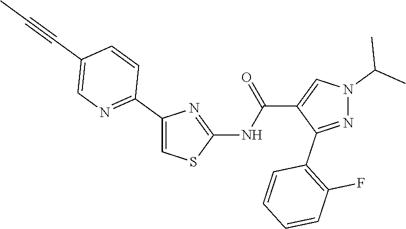 [SMILES: CC#Cc1ccc(-c2csc(NC(=O)c3cn(C(C)C)nc3-c3ccccc3F)n2)nc1]

Acid CAS 1306199-51-8
Amine CAS 2592824-00-3

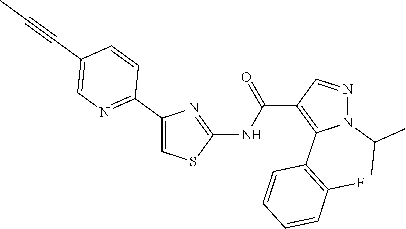 [SMILES: CC#Cc1ccc(-c2csc(NC(=O)c3cnn(C(C)C)c3-c3ccccc3F)n2)nc1]

Acid CAS 1785583-64-3
Amine CAS 2592624-00-3

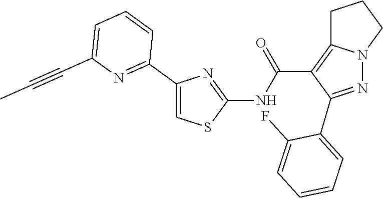 [SMILES: CC#Cc1cccc(-c2csc(NC(=O)c3c(-c4ccccc4F)nn4c3CCC4)n2)n1]

Acid CAS 1338582-57-2
Amine CAS 260 [symbol] 157-36-4

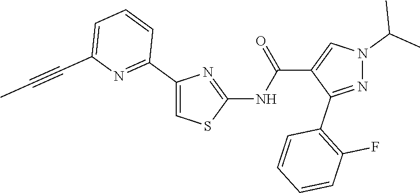 [SMILES: CC#Cc1cccc(-c2csc(NC(=O)c3cn(C(C)C)nc3-c3ccccc3F)n2)n1]

Acid CAS 1306199-51-8
Amine CAS 260 [symbol] 157-36-4

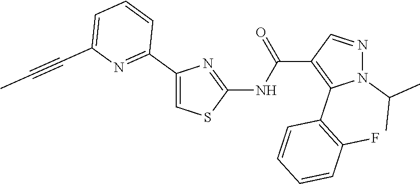 [SMILES: CC#Cc1cccc(-c2csc(NC(=O)c3cnn(C(C)C)c3-c3ccccc3F)n2)n1]

Acid CAS 1765583-64-3
Amine CAS 2600157-36-4

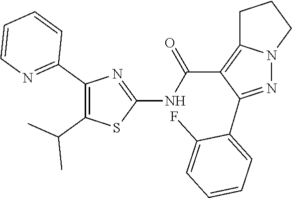 [SMILES: CC(C)c1sc(NC(=O)c2c(-c3ccccc3F)nn3c2CCC3)nc1-c1ccccn1]

Acid CAS 1338582-57-2
Amine CAS 1225774-92-4

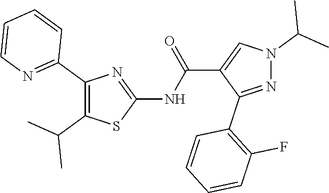 [SMILES: CC(C)c1sc(NC(=O)c2cn(C(C)C)nc2-c2ccccc2F)nc1-c1ccccn1]

Acid CAS 1306199-51-8
Amine CAS 1225774-92-4

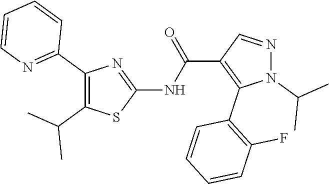 [SMILES: CC(C)c1sc(NC(=O)c2cnn(C(C)C)c2-c2ccccc2F)nc1-c1ccccn1]

Acid CAS 1785563-64-3
Amine CAS 1225774-92-4

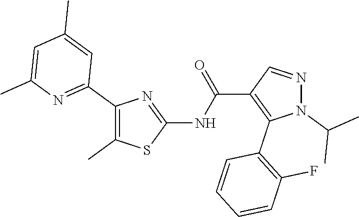 [SMILES: Cc1cc(C)nc(-c2nc(NC(=O)c3cnn(C(C)C)c3-c3ccccc3F)sc2C)c1]

Acid CAS 1785583-64-3
Amine CAS 2287501-11-3

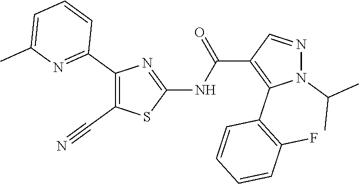 [SMILES: Cc1cccc(-c2nc(NC(=O)c3cnn(C(C)C)c3-c3ccccc3F)sc2C#N)n1]

Acid CAS 1785583-64-3
Amine CAS 1975266-69-3

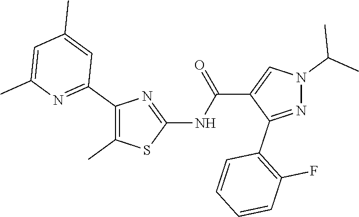 [SMILES: Cc1cc(C)nc(-c2nc(NC(=O)c3cn(C(C)C)nc3-c3ccccc3F)sc2C)c1]

Acid CAS 1306199-51-8
Amine CAS 2287501-11-3

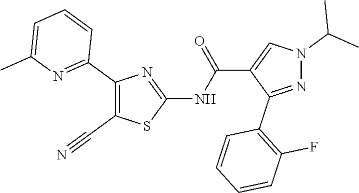 [SMILES: Cc1cccc(-c2nc(NC(=O)c3cn(C(C)C)nc3-c3ccccc3F)sc2C#N)n1]

Acid CAS 1306199-51-8
Amine CAS 1975266-69-3

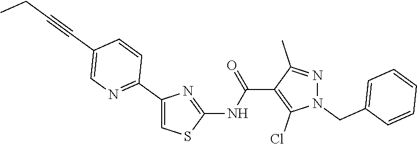 [SMILES: CCC#Cc1ccc(-c2csc(NC(=O)c3c(C)nn(Cc4ccccc4)c3Cl)n2)nc1]

Acid CAS 882227-05-6
Amine CAS 2538808-37-4

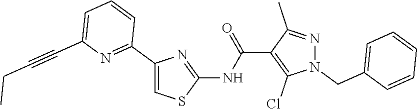 [SMILES: CCC#Cc1cccc(-c2csc(NC(=O)c3c(C)nn(Cc4ccccc4)c3Cl)n2)n1]

Acid CAS 882227-05-8
Amine CAS 2539204-54-9

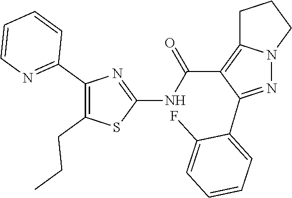 [SMILES: CCCc1sc(NC(=O)c2c(-c3ccccc3F)nn3c2CCC3)nc1-c1ccccn1]

Acid CAS 1338582-57-2
Amine CAS 2042701-33-5

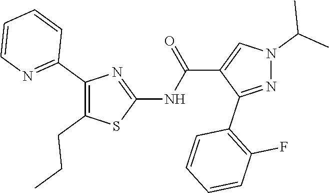 [SMILES: CCCc1sc(NC(=O)c2cn(C(C)C)nc2-c2ccccc2F)nc1-c1ccccn1]

Acid CAS 1306199-51-8
Amine CAS 2042701-33-5

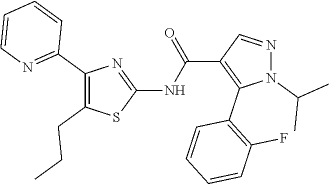 [SMILES: CCCc1sc(NC(=O)c2cnn(C(C)C)c2-c2ccccc2F)nc1-c1ccccn1]

Acid CAS 1785583-64-3
Amine CAS 2042701-33-5

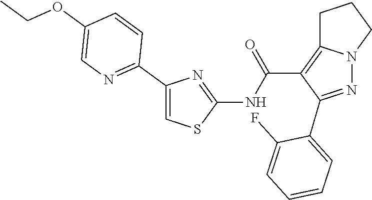 [SMILES: CCOc1ccc(-c2csc(NC(=O)c3c(-c4ccccc4F)nn4c3CCC4)n2)nc1]

Acid CAS 1338582-57-2
Amine CAS 2768587-25-1

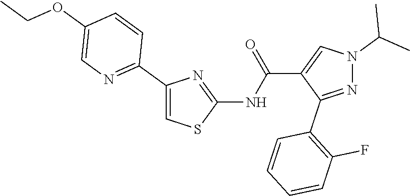 [SMILES: CCOc1ccc(-c2csc(NC(=O)c3cn(C(C)C)nc3-c3ccccc3F)n2)nc1]

Acid CAS 1306199-51-6
Amine CAS 2768587-25-1

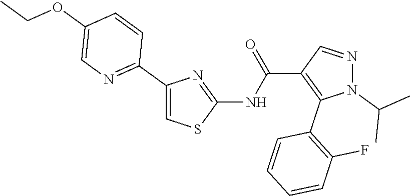 [SMILES: CCOc1ccc(-c2csc(NC(=O)c3cnn(C(C)C)c3-c3ccccc3F)n2)nc1]

Acid CAS 1785583-64-3
Amine CAS 2768587-25-1

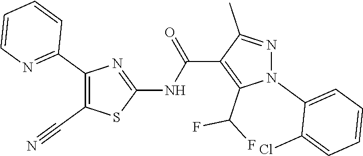 [SMILES: Cc1nn(-c2ccccc2Cl)c(C(F)F)c1C(=O)Nc1nc(-c2ccccn2)c(C#N)s1]

Acid CAS 1375473-93-0
Amine CAS 1139743-06-8

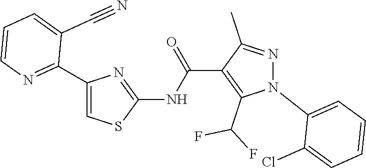 [SMILES: Cc1nn(-c2ccccc2Cl)c(C(F)F)c1C(=O)Nc1nc(-c2ncccc2C#N)cs1]

Acid CAS 1375473-93-0
Amine CAS 2253868-93-6

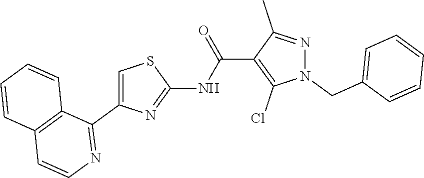 [SMILES: Cc1nn(Cc2ccccc2)c(Cl)c1C(=O)Nc1nc(-c2nccc3ccccc23)cs1]

Acid CAS 882227-05-6
Amine CAS 651042-73-8

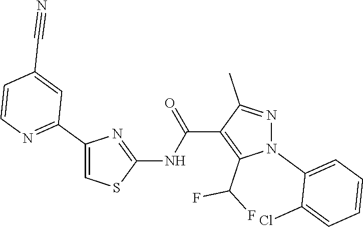 [SMILES: Cc1nn(-c2ccccc2Cl)c(C(F)F)c1C(=O)Nc1nc(-c2cc(C#N)ccn2)cs1]

Acid CAS 1375473-93-0
Amine CAS 2253868-97-0

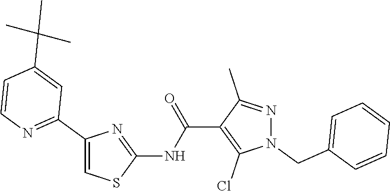 [SMILES: Cc1nn(Cc2ccccc2)c(Cl)c1C(=O)Nc1nc(-c2cc(C(C)(C)C)ccn2)cs1]

Acid CAS 882227-05-6
Amine CAS 2253868-94-7

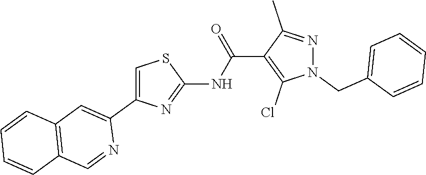 [SMILES: Cc1nn(Cc2ccccc2)c(Cl)c1C(=O)Nc1nc(-c2cc3ccccc3cn2)cs1]

Acid CAS 882227-05-6
Amine CAS 2742888-53-3

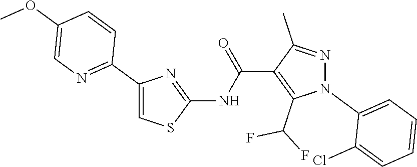 [SMILES: COc1ccc(-c2csc(NC(=O)c3c(C)nn(-c4ccccc4Cl)c3C(F)F)n2)nc1]

Acid CAS 1375473-93-0
Amine CAS 2228346-85-6

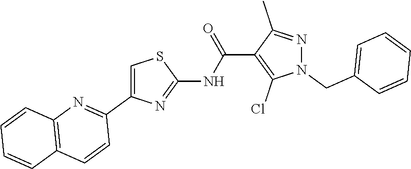 [SMILES: Cc1nn(Cc2ccccc2)c(Cl)c1C(=O)Nc1nc(-c2ccc3ccccc3n2)cs1]

Acid CAS 882227-05-6
Amine CAS 30235-29-1

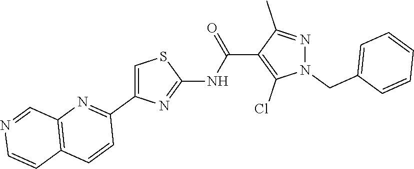 [SMILES: Cc1nn(Cc2ccccc2)c(Cl)c1C(=O)Nc1nc(-c2ccc3ccncc3n2)cs1]

Acid CAS 882227-05-6
Amine CAS 2604301-23-5

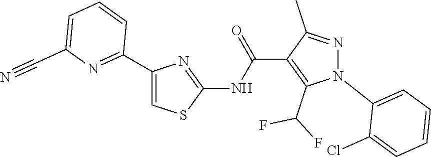 [SMILES: Cc1nn(-c2ccccc2Cl)c(C(F)F)c1C(=O)Nc1nc(-c2cccc(C#N)n2)cs1]

Acid CAS 1375473-93-0
Amine CAS 1185317-90-1

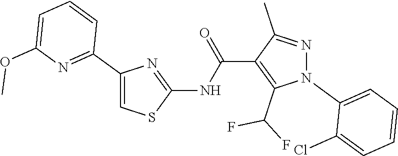 [SMILES: COc1cccc(-c2csc(NC(=O)c3c(C)nn(-c4ccccc4Cl)c3C(F)F)n2)n1]

Acid CAS 1375473-93-0
Amine CAS 1870028-77-5

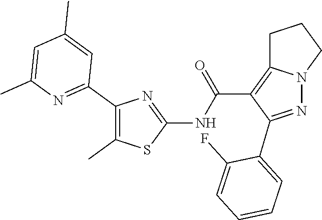 [SMILES: Cc1cc(C)nc(-c2nc(NC(=O)c3c(-c4ccccc4F)nn4c3CCC4)sc2C)c1]

Acid CAS 1338582-57-2
Amine CAS 2287501-11-3

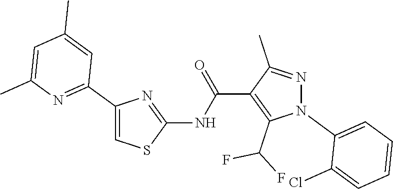 [SMILES: Cc1cc(C)nc(-c2csc(NC(=O)c3c(C)nn(-c4ccccc4Cl)c3C(F)F)n2)c1]

Acid CAS 1375473-93-0
Amine CAS 1865056-88-7

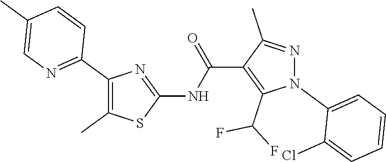 [SMILES: Cc1ccc(-c2nc(NC(=O)c3c(C)nn(-c4ccccc4Cl)c3C(F)F)sc2C)nc1]

Acid CAS 1375473-93-0
Amine CAS 2294596-48-6

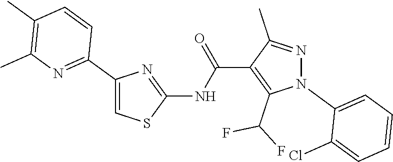 [SMILES: Cc1ccc(-c2csc(NC(=O)c3c(C)nn(-c4ccccc4Cl)c3C(F)F)n2)nc1C]

Acid CAS 1375473-93-0
Amine CAS 1934459-93-4

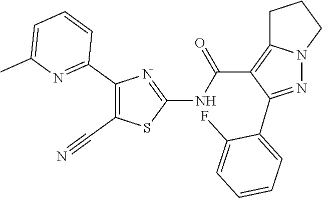 [SMILES: Cc1cccc(-c2nc(NC(=O)c3c(-c4ccccc4F)nn4c3CCC4)sc2C#N)n1]

Acid CAS 1338582-57-2
Amine CAS 1975260-69-3

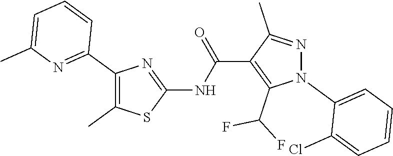 [SMILES: Cc1cccc(-c2nc(NC(=O)c3c(C)nn(-c4ccccc4Cl)c3C(F)F)sc2C)n1]

Acid CAS 1375473-93-0
Amine CAS 2169472-77-7

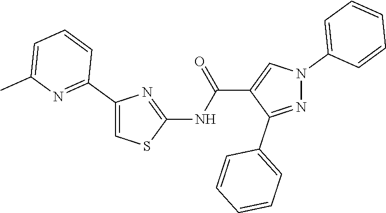 [SMILES: Cc1cccc(-c2csc(NC(=O)c3cn(-c4ccccc4)nc3-c3ccccc3)n2)n1]

Acid CAS 77169-12-1
Amine CAS 99358-99-3

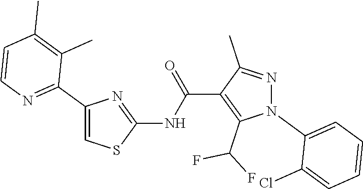 [SMILES: Cc1ccnc(-c2csc(NC(=O)c3c(C)nn(-c4ccccc4Cl)c3C(F)F)n2)c1C]

Acid CAS 1375473-93-0
Amine CAS 2169166-95-2

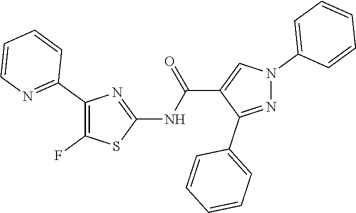 [SMILES: O=C(Nc1nc(-c2ccccn2)c(F)s1)c1cn(-c2ccccc2)nc1-c1ccccc1]

Acid CAS 77169-12-1
Amine CAS 1044270-93-0

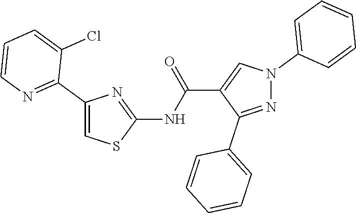 [SMILES: O=C(Nc1nc(-c2ncccc2Cl)cs1)c1cn(-c2ccccc2)nc1-c1ccccc1]

Acid CAS 77169-12-1
Amine CAS 2150665-06-6

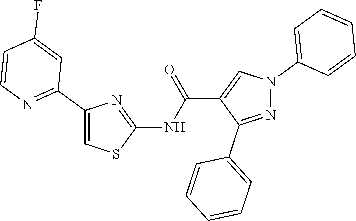 [SMILES: O=C(Nc1nc(-c2cc(F)ccn2)cs1)c1cn(-c2ccccc2)nc1-c1ccccc1]

Acid CAS 77169-12-1
Amine CAS 2228222-84-0

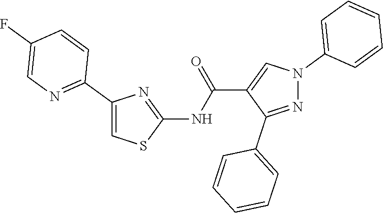 [SMILES: O=C(Nc1nc(-c2ccc(F)cn2)cs1)c1cn(-c2ccccc2)nc1-c1ccccc1]

Acid CAS 77169-12-1
Amine CAS 1551651-30-9

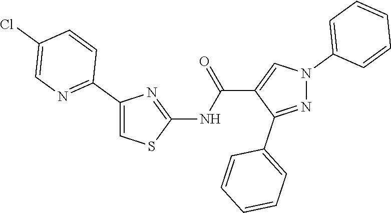 [SMILES: O=C(Nc1nc(-c2ccc(Cl)cn2)cs1)c1cn(-c2ccccc2)nc1-c1ccccc1]

Acid CAS 77169-12-1
Amine CAS 1185315-17-6

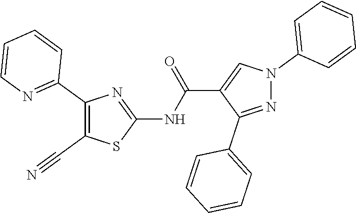 [SMILES: N#Cc1sc(NC(=O)c2cn(-c3ccccc3)nc2-c2ccccc2)nc1-c1ccccn1]

Acid CAS 77169-12-1
Amine CAS 1139743-06-8

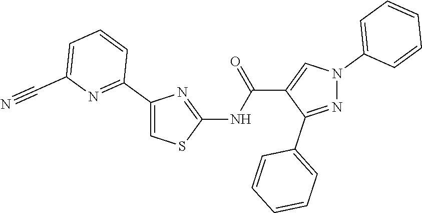 [SMILES: N#Cc1cccc(-c2csc(NC(=O)c3cn(-c4ccccc4)nc3-c3ccccc3)n2)n1]

Acid CAS 77169-12-1
Amine CAS 1185317-90-1

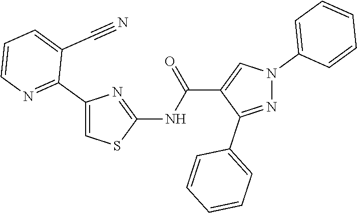 [SMILES: N#Cc1cccnc1-c1csc(NC(=O)c2cn(-c3ccccc3)nc2-c2ccccc2)n1]

Acid CAS 77169-12-1
Amine CAS 2253868-93-6

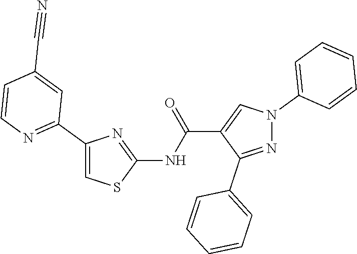 [SMILES: N#Cc1ccnc(-c2csc(NC(=O)c3cn(-c4ccccc4)nc3-c3ccccc3)n2)c1]

Acid CAS 77169-12-1
Amine CAS 2253868-97-0

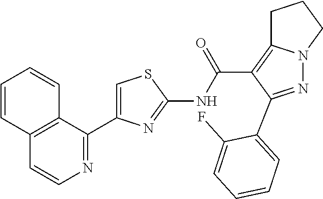 [SMILES: O=C(Nc1nc(-c2nccc3ccccc23)cs1)c1c(-c2ccccc2F)nn2c1CCC2]

Acid CAS 133 [symbol] 582-57-2
Amine CAS 651042-73-8

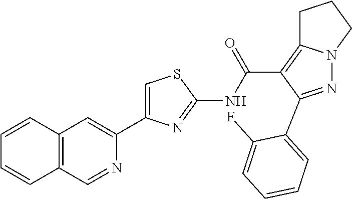 [SMILES: O=C(Nc1nc(-c2cc3ccccc3cn2)cs1)c1c(-c2ccccc2F)nn2c1CCC2]

Acid CAS 1338582-57-2
Amine CAS 2742888-53-3

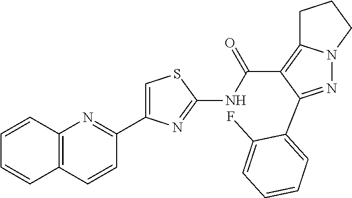 [SMILES: O=C(Nc1nc(-c2ccc3ccccc3n2)cs1)c1c(-c2ccccc2F)nn2c1CCC2]

Acid CAS 1338582-57-2
Amine CAS 30235-29-1

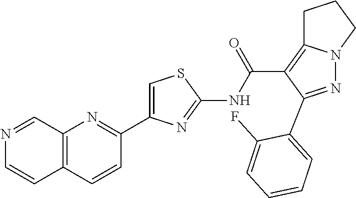 [SMILES: O=C(Nc1nc(-c2ccc3ccncc3n2)cs1)c1c(-c2ccccc2F)nn2c1CCC2]

Acid CAS 1338582-57-2
Amine CAS 2604301-23-5

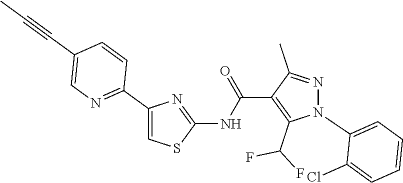 [SMILES: CC#Cc1ccc(-c2csc(NC(=O)c3c(C)nn(-c4ccccc4Cl)c3C(F)F)n2)nc1]

Acid CAS 1375473-93-0
Amine CAS 2592824-00-3

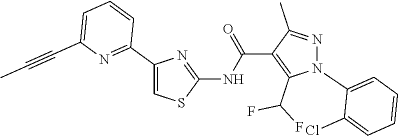 [SMILES: CC#Cc1cccc(-c2csc(NC(=O)c3c(C)nn(-c4ccccc4Cl)c3C(F)F)n2)n1]

Acid CAS 1375473-93-0
Amine CAS 2600157-36-4

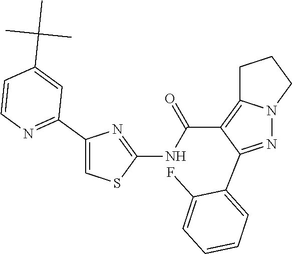 [SMILES: CC(C)(C)c1ccnc(-c2csc(NC(=O)c3c(-c4ccccc4F)nn4c3CCC4)n2)c1]

Acid CAS 1338582-57-2
Amine CAS 2253868-94-7

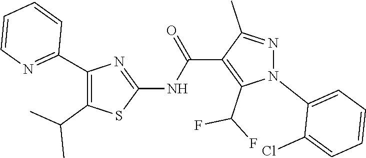 [SMILES: Cc1nn(-c2ccccc2Cl)c(C(F)F)c1C(=O)Nc1nc(-c2ccccn2)c(C(C)C)s1]

Acid CAS 1375473-93-0
Amine CAS 1225774-92-4

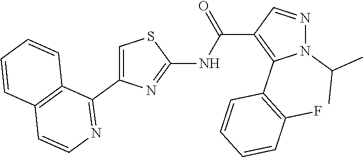 [SMILES: CC(C)n1ncc(C(=O)Nc2nc(-c3nccc4ccccc34)cs2)c1-c1ccccc1F]

Acid CAS 178 [symbol] 583-64-3
Amine CAS 651042-73-6

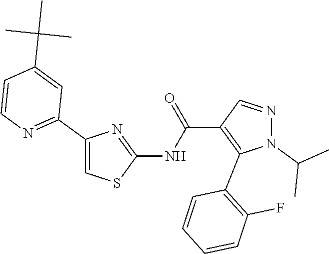 [SMILES: CC(C)n1ncc(C(=O)Nc2nc(-c3cc(C(C)(C)C)ccn3)cs2)c1-c1ccccc1F]

Acid CAS 1785583-64-3
Amine CAS 2253868-94-7

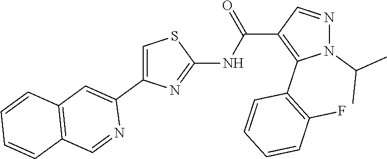 [SMILES: CC(C)n1ncc(C(=O)Nc2nc(-c3cc4ccccc4cn3)cs2)c1-c1ccccc1F]

Acid CAS 1785583-64-3
Amine CAS 2742888-53-3

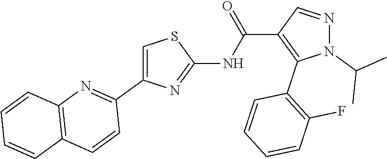 [SMILES: CC(C)n1ncc(C(=O)Nc2nc(-c3ccc4ccccc4n3)cs2)c1-c1ccccc1F]

Acid CAS 1785583-64-3
Amine CAS 30235-29-1

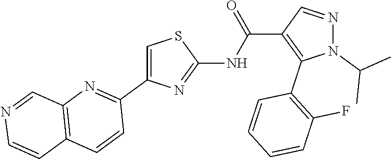 [SMILES: CC(C)n1ncc(C(=O)Nc2nc(-c3ccc4ccncc4n3)cs2)c1-c1ccccc1F]

Acid CAS 1785583-64-3
Amine CAS 2604301-23-5

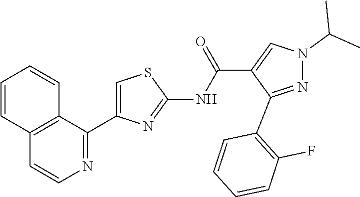 [SMILES: CC(C)n1cc(C(=O)Nc2nc(-c3nccc4ccccc34)cs2)c(-c2ccccc2F)n1]

Acid CAS 1306199-51-8
Amine CAS [symbol] 51042-73-8

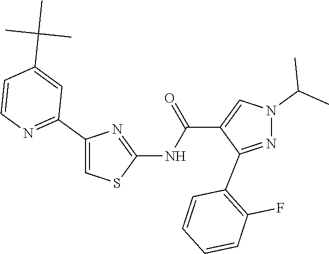 [SMILES: CC(C)n1cc(C(=O)Nc2nc(-c3cc(C(C)(C)C)ccn3)cs2)c(-c2ccccc2F)n1]

Acid CAS 1306199-51- [symbol]
Amine CAS 2253868-94-7

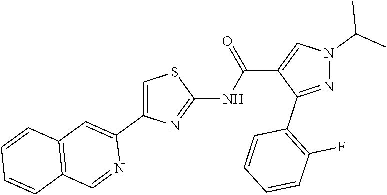 [SMILES: CC(C)n1cc(C(=O)Nc2nc(-c3cc4ccccc4cn3)cs2)c(-c2ccccc2F)n1]

Acid CAS 1306199-51-8
Amine CAS 2742888-53-3

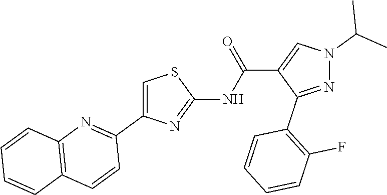 [SMILES: CC(C)n1cc(C(=O)Nc2nc(-c3ccc4ccccc4n3)cs2)c(-c2ccccc2F)n1]

Acid CAS 1306199-51-8
Amine CAS 30235-29-1

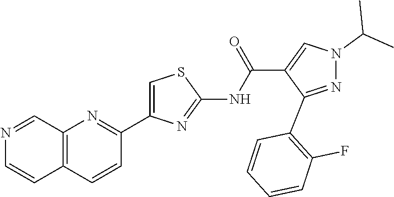 [SMILES: CC(C)n1cc(C(=O)Nc2nc(-c3ccc4ccncc4n3)cs2)c(-c2ccccc2F)n1]

Acid CAS 1306199-51-8
Amine CAS 2604301-23-5

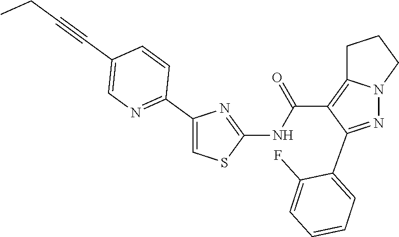 [SMILES: CCC#Cc1ccc(-c2csc(NC(=O)c3c(-c4ccccc4F)nn4c3CCC4)n2)nc1]

Acid CAS 1338582-57-2
Amine CAS 2538808-37-4

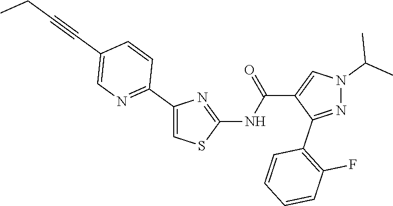 [SMILES: CCC#Cc1ccc(-c2csc(NC(=O)c3cn(C(C)C)nc3-c3ccccc3F)n2)nc1]

Acid CAS 1306199-51-8
Amine CAS 2538808-37-4

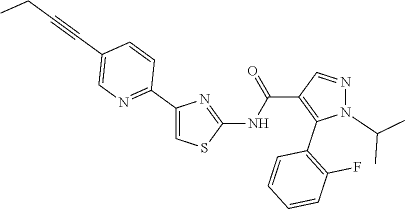 [SMILES: CCC#Cc1ccc(-c2csc(NC(=O)c3cnn(C(C)C)c3-c3ccccc3F)n2)nc1]

Acid CAS 1785583-64-3
Amine CAS 2538808-37-4

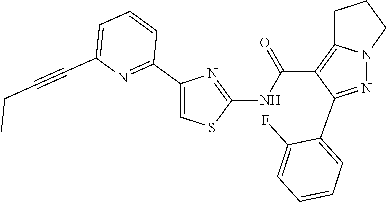 [SMILES: CCC#Cc1cccc(-c2csc(NC(=O)c3c(-c4ccccc4F)nn4c3CCC4)n2)n1]

Acid CAS 1338582-57-2
Amine CAS 2539204-54-9

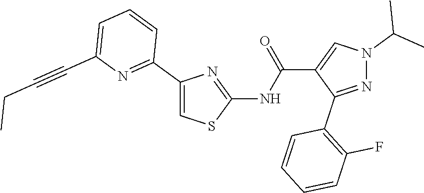 [SMILES: CCC#Cc1cccc(-c2csc(NC(=O)c3cn(C(C)C)nc3-c3ccccc3F)n2)n1]

Acid CAS 1306199-51-8
Amine CAS 2539204-54-9

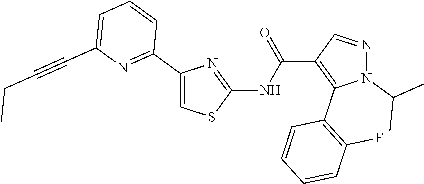 [SMILES: CCC#Cc1cccc(-c2csc(NC(=O)c3cnn(C(C)C)c3-c3ccccc3F)n2)n1]

Acid CAS 1785583-64-3
Amine CAS 2539204-54-9

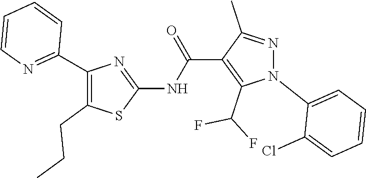 [SMILES: CCCc1sc(NC(=O)c2c(C)nn(-c3ccccc3Cl)c2C(F)F)nc1-c1ccccn1]

Acid CAS 1375473-93-0
Amine CAS 2042701-33-5

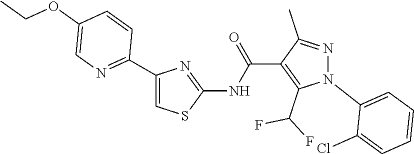 [SMILES: CCOc1ccc(-c2csc(NC(=O)c3c(C)nn(-c4ccccc4Cl)c3C(F)F)n2)nc1]

Acid CAS 1375473-93-0
Amine CAS 2768587-2 [symbol] 1

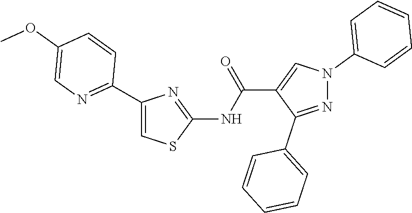 [SMILES: COc1ccc(-c2csc(NC(=O)c3cn(-c4ccccc4)nc3-c3ccccc3)n2)nc1]

Acid CAS 77169-12-1
Amine CAS 2228346-85-6

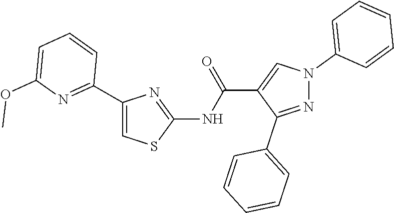 [SMILES: COc1cccc(-c2csc(NC(=O)c3cn(-c4ccccc4)nc3-c3ccccc3)n2)n1]

Acid CAS 77169-12-1
Amine CAS 1870028-77-5

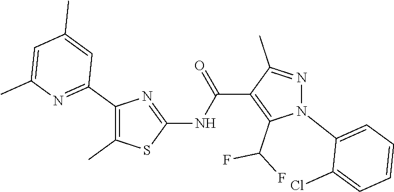 [SMILES: Cc1cc(C)nc(-c2nc(NC(=O)c3c(C)nn(-c4ccccc4Cl)c3C(F)F)sc2C)c1]

Acid CAS 1375473-93-0
Amine CAS 2287501-11-3

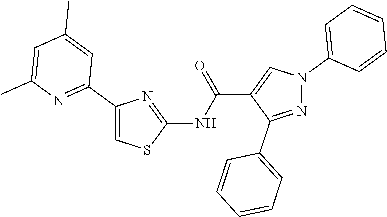 [SMILES: Cc1cc(C)nc(-c2csc(NC(=O)c3cn(-c4ccccc4)nc3-c3ccccc3)n2)c1]

Acid CAS 77169-12-1
Amine CAS 1865 [symbol] 56-88-7

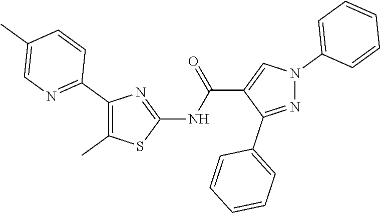 [SMILES: Cc1ccc(-c2nc(NC(=O)c3cn(-c4ccccc4)nc3-c3ccccc3)sc2C)nc1]

Acid CAS 77169-12-1
Amine CAS 2294596-48-6

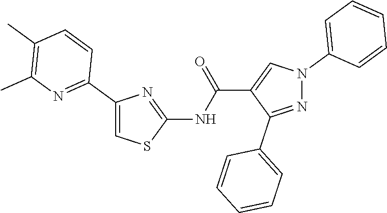 [SMILES: Cc1ccc(-c2csc(NC(=O)c3cn(-c4ccccc4)nc3-c3ccccc3)n2)nc1C]

Acid CAS 77169-12-1
Amine CAS 1934459-93-4

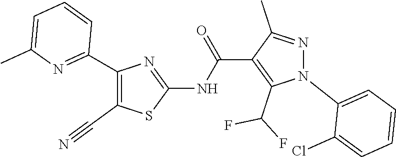 [SMILES: Cc1cccc(-c2nc(NC(=O)c3c(C)nn(-c4ccccc4Cl)c3C(F)F)sc2C#N)n1]

Acid CAS 1375473-93-0
Amine CAS 197566-69-3

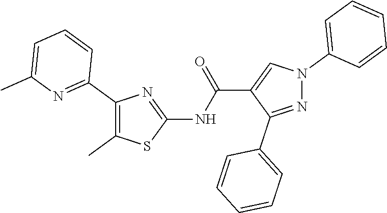 [SMILES: Cc1cccc(-c2nc(NC(=O)c3cn(-c4ccccc4)nc3-c3ccccc3)sc2C)n1]

Acid CAS 77169-12-1
Amine CAS 2169472-77-7

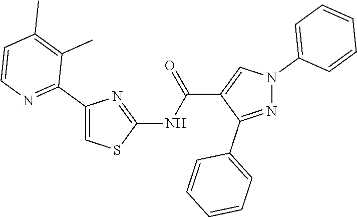 [SMILES: Cc1ccnc(-c2csc(NC(=O)c3cn(-c4ccccc4)nc3-c3ccccc3)n2)c1C]

Acid CAS 77169-12-1
Amine CAS 2169166-95-2

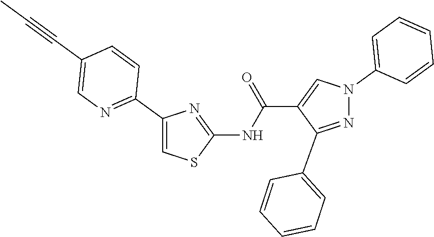 [SMILES: CC#Cc1ccc(-c2csc(NC(=O)c3cn(-c4ccccc4)nc3-c3ccccc3)n2)nc1]

Acid CAS 77169-12-1
Amine CAS 25928424-00-3

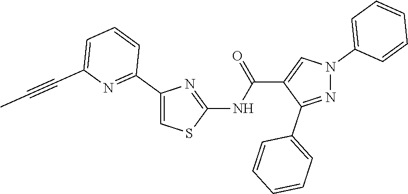 [SMILES: CC#Cc1cccc(-c2csc(NC(=O)c3cn(-c4ccccc4)nc3-c3ccccc3)n2)n1]

Acid CAS 77169-12-1
Amine CAS 2600157-36-4

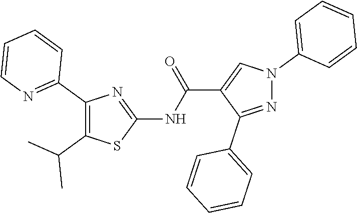 [SMILES: CC(C)c1sc(NC(=O)c2cn(-c3ccccc3)nc2-c2ccccc2)nc1-c1ccccn1]

Acid CAS 77169-12-1
Amine CAS 1225774-92-4

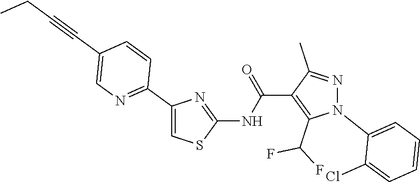 [SMILES: CCC#Cc1ccc(-c2csc(NC(=O)c3c(C)nn(-c4ccccc4Cl)c3C(F)F)n2)nc1]

Acid CAS 1375473-93-0
Amine CAS 2538808-37-4

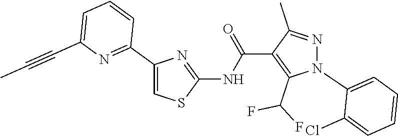 [SMILES: CC#Cc1cccc(-c2csc(NC(=O)c3c(C)nn(-c4ccccc4Cl)c3C(F)F)n2)n1]

Acid CAS 1375473-93-0
Amine CAS 2539204-54-9

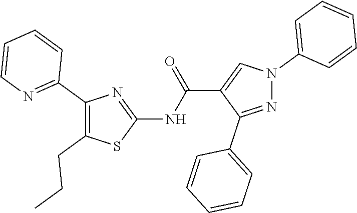 [SMILES: CCCc1sc(NC(=O)c2cn(-c3ccccc3)nc2-c2ccccc2)nc1-c1ccccn1]

Acid CAS 77169-12-1
Amine CAS 2042701-33-5

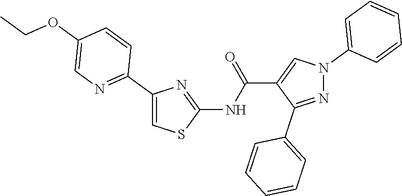 [SMILES: CCOc1ccc(-c2csc(NC(=O)c3cn(-c4ccccc4)nc3-c3ccccc3)n2)nc1]

Acid CAS 77169-12-1
Amine CAS 2766587-25-1

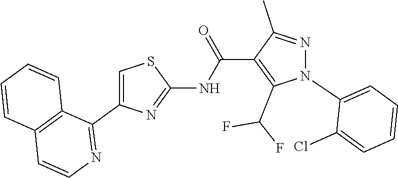 [SMILES: Cc1nn(-c2ccccc2Cl)c(C(F)F)c1C(=O)Nc1nc(-c2nccc3ccccc23)cs1]

Acid CAS 1375473-93-0
Amine CAS 651042-73-8

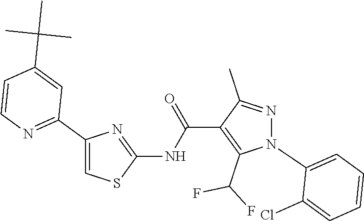 [SMILES: Cc1nn(-c2ccccc2Cl)c(C(F)F)c1C(=O)Nc1nc(-c2cc(C(C)(C)C)ccn2)cs1]

Acid CAS 1375473-93-0
Amine CAS 2253868-94-7

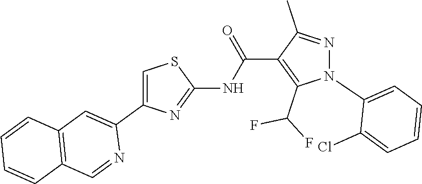 [SMILES: Cc1nn(-c2ccccc2Cl)c(C(F)F)c1C(=O)Nc1nc(-c2cc3ccccc3cn2)cs1]

Acid CAS 1375473-93-0
Amine CAS 274868-53-3

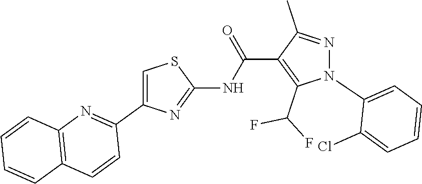 [SMILES: Cc1nn(-c2ccccc2Cl)c(C(F)F)c1C(=O)Nc1nc(-c2ccc3ccccc3n2)cs1]

Acid CAS 1375473-93-0
Amine CAS 30235-29-1

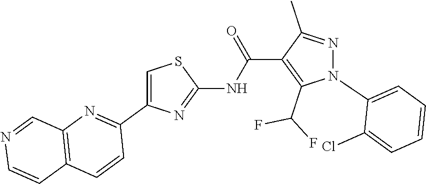 [SMILES: Cc1nn(-c2ccccc2Cl)c(C(F)F)c1C(=O)Nc1nc(-c2ccc3ccncc3n2)cs1]

Acid CAS 1375473-93-0
Amine CAS 2604301-23-5

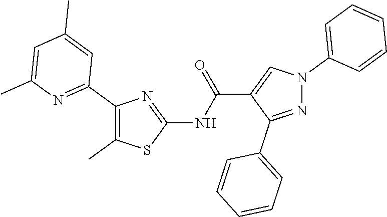 [SMILES: Cc1cc(C)nc(-c2nc(NC(=O)c3cn(-c4ccccc4)nc3-c3ccccc3)sc2C)c1]

Acid CAS 77169-12-1
Amine CAS 2287501-11-3

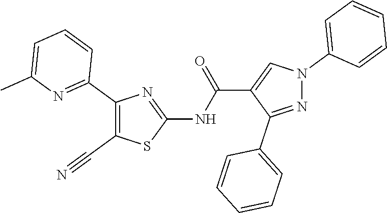 [SMILES: Cc1cccc(-c2nc(NC(=O)c3cn(-c4ccccc4)nc3-c3ccccc3)sc2C#N)n1]

Acid CAS 77169-12-1
Amine CAS 1975266-69-3

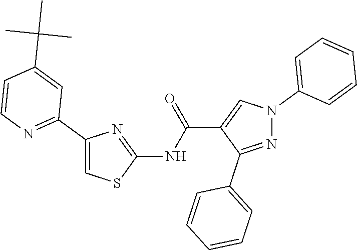 [SMILES: CC(C)(C)c1ccnc(-c2csc(NC(=O)c3cn(-c4ccccc4)nc3-c3ccccc3)n2)c1]

Acid CAS 77169-12-1
Amine CAS 2253868-94-7

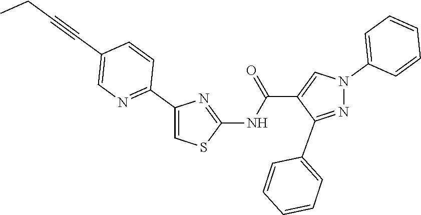 [SMILES: CCC#Cc1ccc(-c2csc(NC(=O)c3cn(-c4ccccc4)nc3-c3ccccc3)n2)nc1]

Acid CAS 77169-12-1
Amine CAS 2538 [symbol] 08-37-4

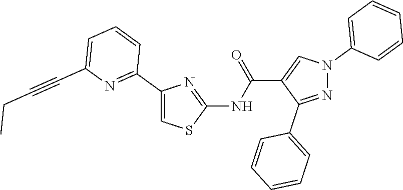 [SMILES: CCC#Cc1cccc(-c2csc(NC(=O)c3cn(-c4ccccc4)nc3-c3ccccc3)n2)n1]

Acid CAS 77169-12-1
Amine CAS 2539204-54-9

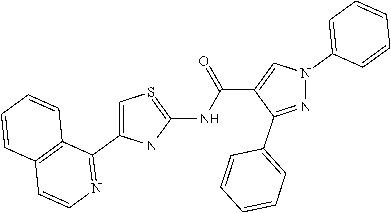

Acid CAS 77169-12-1
Amine CAS 651042-73-6

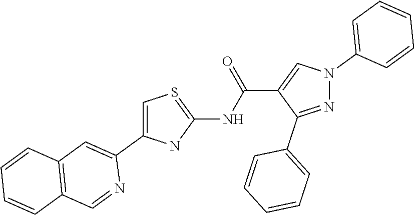

Acid CAS 77169-12-1
Amine CAS 2742886-53-3

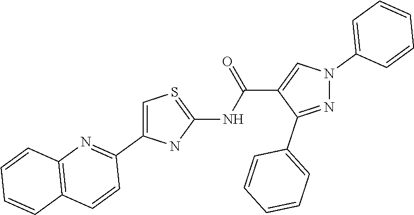

Acid CAS 77169-12-1
Amine CAS 302235-29-1

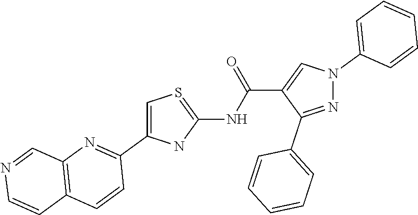

Acid CAS 77169-12-1
Amine CAS 2684301-23-5

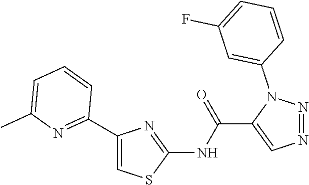 [SMILES: Cc1cccc(-c2csc(NC(=O)c3cnnn3-c3cccc(F)c3)n2)n1]

Acid CAS 1153490-92- [symbol]
Amine CAS 99358-99-3

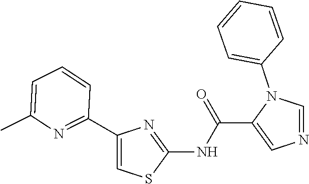 [SMILES: Cc1cccc(-c2csc(NC(=O)c3cncn3-c3ccccc3)n2)n1]

Acid CAS 135417-65-1
Amine CAS 99358-99-3

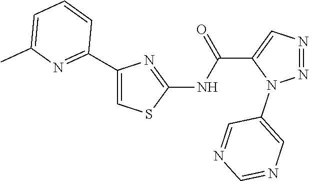 [SMILES: Cc1cccc(-c2csc(NC(=O)c3cnnn3-c3cncnc3)n2)n1]

Acid CAS 147 [symbol] 093-94-5
Amine CAS 99358-99-3

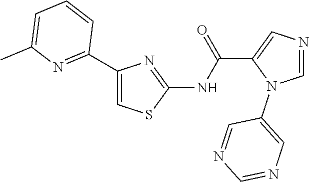 [SMILES: Cc1cccc(-c2csc(NC(=O)c3cncn3-c3cncnc3)n2)n1]

Acid CAS 1523297-19-9
Amine CAS 99358-99-3

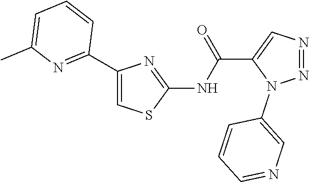 [SMILES: Cc1cccc(-c2csc(NC(=O)c3cnnn3-c3cccnc3)n2)n1]

Acid CAS 1476271-10-1
Amine CAS 99358-99-3

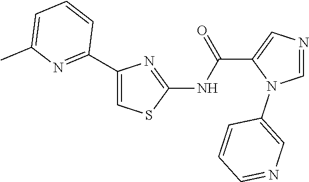 [SMILES: Cc1cccc(-c2csc(NC(=O)c3cncn3-c3cccnc3)n2)n1]

Acid CAS 1197398-67-6
Amine CAS 99358-99-3

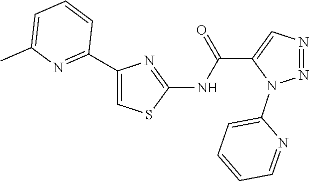 [SMILES: Cc1cccc(-c2csc(NC(=O)c3cnnn3-c3ccccn3)n2)n1]

Acid CAS 1480445-52-0
Amine CAS 99358-99-3

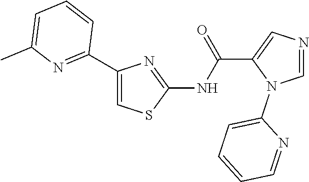 [SMILES: Cc1cccc(-c2csc(NC(=O)c3cncn3-c3ccccn3)n2)n1]

Acid CAS 1779128-15-2
Amine CAS 99358-99-3

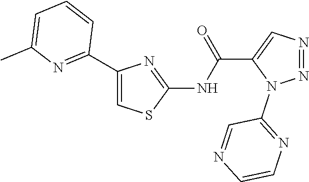 [SMILES: Cc1cccc(-c2csc(NC(=O)c3cnnn3-c3cnccn3)n2)n1]

Acid CAS 2946657-15-2
Amine CAS 99358-99-3

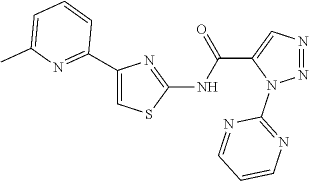 [SMILES: Cc1cccc(-c2csc(NC(=O)c3cnnn3-c3ncccn3)n2)n1]

Acid CAS 2946657-14-1
Amine CAS 99358-99-3

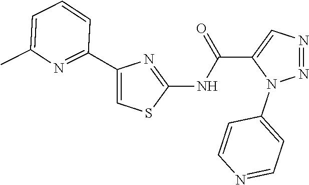 [SMILES: Cc1cccc(-c2csc(NC(=O)c3cnnn3-c3ccncc3)n2)n1]

Acid CAS 1525417-94-0
Amine CAS 99358-99-3

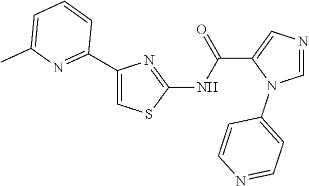 [SMILES: Cc1cccc(-c2csc(NC(=O)c3cncn3-c3ccncc3)n2)n1]

Acid CAS 1439960-49-8
Amine CAS 99358-99-3

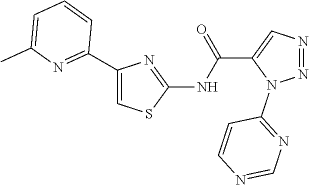 [SMILES: Cc1cccc(-c2csc(NC(=O)c3cnnn3-c3ccncn3)n2)n1]

Acid CAS 1482934-49-5
Amine CAS 99358-99-3

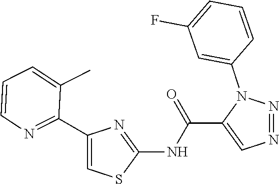 [SMILES: Cc1cccnc1-c1csc(NC(=O)c2cnnn2-c2cccc(F)c2)n1]

Acid CAS 1153490-92-6
Amine CAS 1044269-91-1

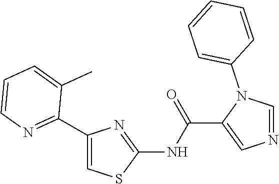 [SMILES: Cc1cccnc1-c1csc(NC(=O)c2cncn2-c2ccccc2)n1]

Acid CAS 135417-65-1
Amine CAS 1044269-91-1

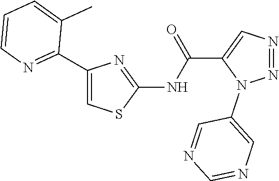 [SMILES: Cc1cccnc1-c1csc(NC(=O)c2cnnn2-c2cncnc2)n1]

Acid CAS 147 [symbol] 093-94-5
Amine CAS 1044269-91-1

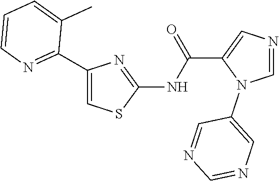 [SMILES: Cc1cccnc1-c1csc(NC(=O)c2cncn2-c2cncnc2)n1]

Acid CAS 1523297-19-9
Amine CAS 1044269-91-1

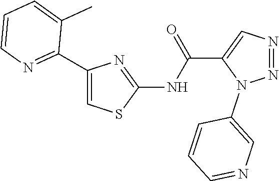 [SMILES: Cc1cccnc1-c1csc(NC(=O)c2cnnn2-c2cccnc2)n1]

Acid CAS 1478271-10-1
Amine CAS 1044269-91-1

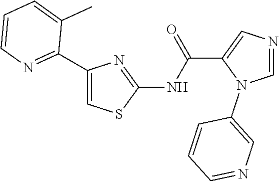 [SMILES: Cc1cccnc1-c1csc(NC(=O)c2cncn2-c2cccnc2)n1]

Acid CAS 1197398-67-6
Amine CAS 1044269-91-1

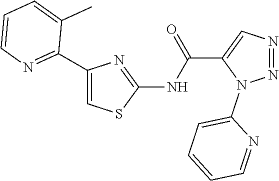 [SMILES: Cc1cccnc1-c1csc(NC(=O)c2cnnn2-c2ccccn2)n1]

Acid CAS 1480445-52-0
Amine CAS 1044269-91-1

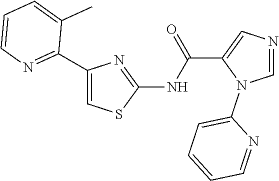 [SMILES: Cc1cccnc1-c1csc(NC(=O)c2cncn2-c2ccccn2)n1]

Acid CAS 1779128-15-2
Amine CAS 1044269-91-1

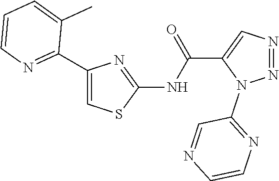 [SMILES: Cc1cccnc1-c1csc(NC(=O)c2cnnn2-c2cnccn2)n1]

Acid CAS 2946657-15-2
Amine CAS 1044269-91-1

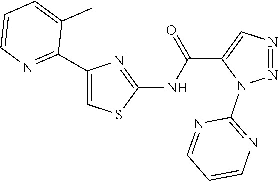 [SMILES: Cc1cccnc1-c1csc(NC(=O)c2cnnn2-c2ncccn2)n1]

Acid CAS 2946657-14-1
Amine CAS 1044269-91-1

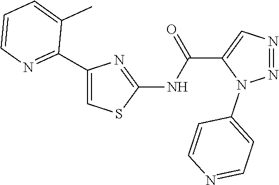 [SMILES: Cc1cccnc1-c1csc(NC(=O)c2cnnn2-c2ccncc2)n1]

Acid CAS 1525417-94-0
Amine CAS 1044269-91-1

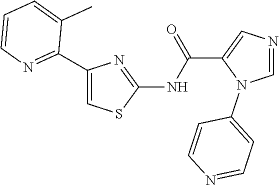 [SMILES: Cc1cccnc1-c1csc(NC(=O)c2cncn2-c2ccncc2)n1]

Acid CAS 1439900-49-8
Amine CAS 1044269-91-1

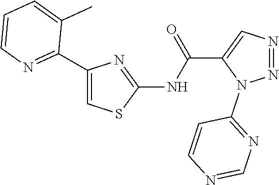 [SMILES: Cc1cccnc1-c1csc(NC(=O)c2cnnn2-c2ccncn2)n1]

Acid CAS 1482934-49-5
Amine CAS 1044269-91-1

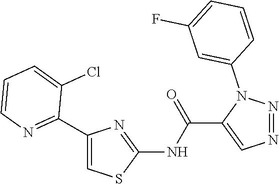 [SMILES: O=C(Nc1nc(-c2ncccc2Cl)cs1)c1cnnn1-c1cccc(F)c1]

Acid CAS 1153490-92-6
Amine CAS 2150665-06-6

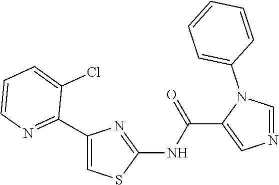 [SMILES: O=C(Nc1nc(-c2ncccc2Cl)cs1)c1cncn1-c1ccccc1]

Acid CAS 135417-65-1
Amine CAS 2150665-06-6

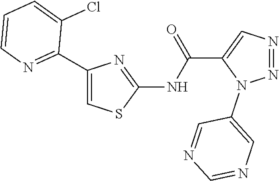 [SMILES: O=C(Nc1nc(-c2ncccc2Cl)cs1)c1cnnn1-c1cncnc1]

Acid CAS 147 [symbol] 093-94-5
Amine CAS 2150665-06-6

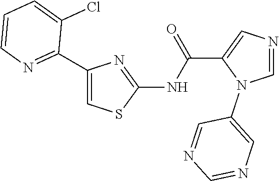 [SMILES: O=C(Nc1nc(-c2ncccc2Cl)cs1)c1cncn1-c1cncnc1]

Acid CAS 1523297-19-9
Amine CAS 2150665-06-6

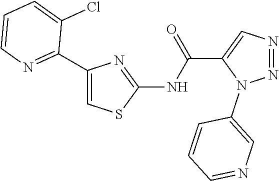 [SMILES: O=C(Nc1nc(-c2ncccc2Cl)cs1)c1cnnn1-c1cccnc1]

Acid CAS 147 [symbol] 271-10-1
Amine CAS 2150665-06-6

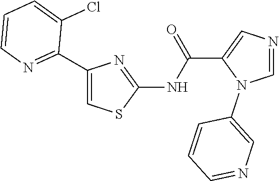 [SMILES: O=C(Nc1nc(-c2ncccc2Cl)cs1)c1cncn1-c1cccnc1]

Acid CAS 1197396-67-6
Amine CAS 2150665-06-6

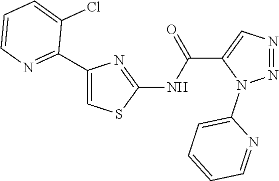 [SMILES: O=C(Nc1nc(-c2ncccc2Cl)cs1)c1cnnn1-c1ccccn1]

Acid CAS 1480445-52-0
Amine CAS 2150665-06-6

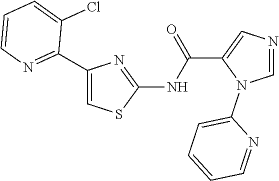 [SMILES: O=C(Nc1nc(-c2ncccc2Cl)cs1)c1cncn1-c1ccccn1]

Acid CAS 1779128-15-2
Amine CAS 2150665-06-6

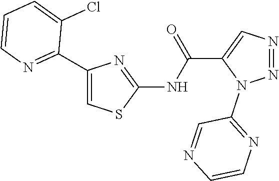 [SMILES: O=C(Nc1nc(-c2ncccc2Cl)cs1)c1cnnn1-c1cnccn1]

Acid CAS 2946657-15-2
Amine CAS 2150665-06-6

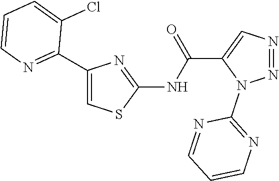 [SMILES: O=C(Nc1nc(-c2ncccc2Cl)cs1)c1cnnn1-c1ncccn1]

Acid CAS 2946657-14-1
Amine CAS 2150665-06-6

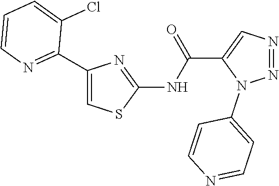 [SMILES: O=C(Nc1nc(-c2ncccc2Cl)cs1)c1cnnn1-c1ccncc1]

Acid CAS 1525417-94-0
Amine CAS 2150665-06-6

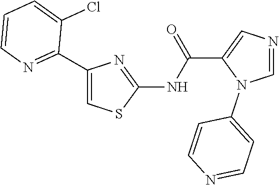 [SMILES: O=C(Nc1nc(-c2ncccc2Cl)cs1)c1cncn1-c1ccncc1]

Acid CAS 1439900-49-8
Amine CAS 2150665-06-6

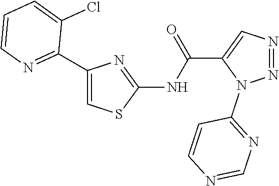 [SMILES: O=C(Nc1nc(-c2ncccc2Cl)cs1)c1cnnn1-c1ccncn1]

Acid CAS 1482934-49-5
Amine CAS 2150665-06-6

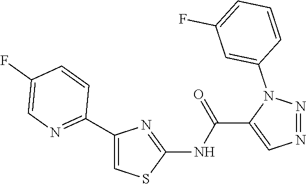 [SMILES: O=C(Nc1nc(-c2ccc(F)cn2)cs1)c1cnnn1-c1cccc(F)c1]

Acid CAS 1153490-92-6
Amine CAS 1551651-30-9

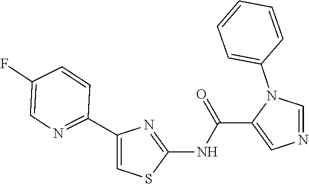 [SMILES: O=C(Nc1nc(-c2ccc(F)cn2)cs1)c1cncn1-c1ccccc1]

Acid CAS 135417-65-1
Amine CAS 1551651-30-9

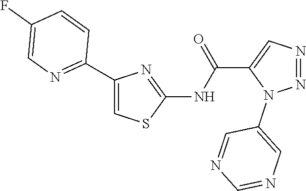 [SMILES: O=C(Nc1nc(-c2ccc(F)cn2)cs1)c1cnnn1-c1cncnc1]

Acid CAS 1476093-94-5
Amine CAS 1551651-30-9

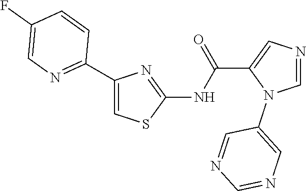 [SMILES: O=C(Nc1nc(-c2ccc(F)cn2)cs1)c1cncn1-c1cncnc1]

Acid CAS 1523297-19-9
Amine CAS 1551651-30-9

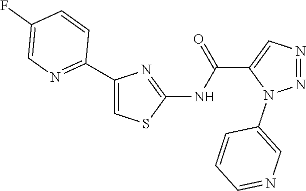 [SMILES: O=C(Nc1nc(-c2ccc(F)cn2)cs1)c1cnnn1-c1cccnc1]

Acid CAS 1476271-10-1
Amine CAS 1551651-30-9

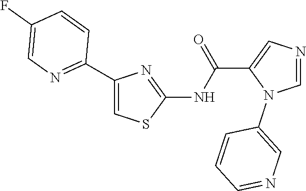 [SMILES: O=C(Nc1nc(-c2ccc(F)cn2)cs1)c1cncn1-c1cccnc1]

Acid CAS 1197398-67-6
Amine CAS 1551651-30-9

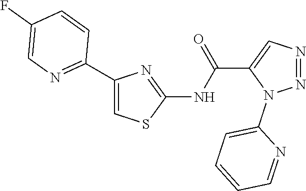 [SMILES: O=C(Nc1nc(-c2ccc(F)cn2)cs1)c1cnnn1-c1ccccn1]

Acid CAS 1480445-52-0
Amine CAS 1551651-30-9

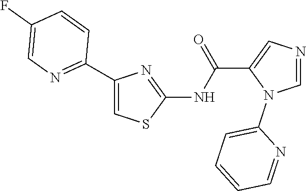 [SMILES: O=C(Nc1nc(-c2ccc(F)cn2)cs1)c1cncn1-c1ccccn1]

Acid CAS 1779128-15-2
Amine CAS 1551651-30-9

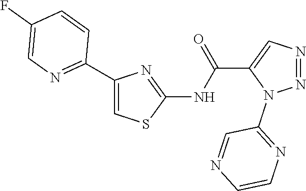 [SMILES: O=C(Nc1nc(-c2ccc(F)cn2)cs1)c1cnnn1-c1cnccn1]

Acid CAS 2946657-15-2
Amine CAS 1551651-30-9

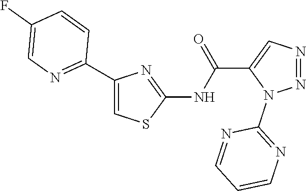 [SMILES: O=C(Nc1nc(-c2ccc(F)cn2)cs1)c1cnnn1-c1ncccn1]

Acid CAS 2946657-14-1
Amine CAS 1551651-30-9

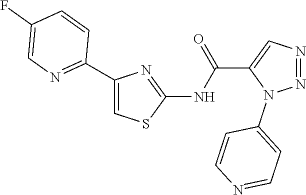 [SMILES: O=C(Nc1nc(-c2ccc(F)cn2)cs1)c1cnnn1-c1ccncc1]

Acid CAS 1525417-94-0
Amine CAS 1551651-30-9

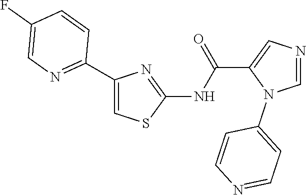 [SMILES: O=C(Nc1nc(-c2ccc(F)cn2)cs1)c1cncn1-c1ccncc1]

Acid CAS 1439900-49-8
Amine CAS 1551651-30-0

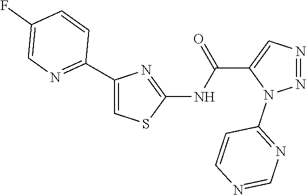 [SMILES: O=C(Nc1nc(-c2ccc(F)cn2)cs1)c1cnnn1-c1ccncn1]

Acid CAS 1482934-49-5
Amine CAS 1551651-30-9

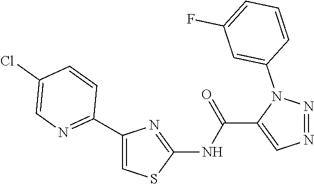 [SMILES: O=C(Nc1nc(-c2ccc(Cl)cn2)cs1)c1cnnn1-c1cccc(F)c1]

Acid CAS 1153490-92-6
Amine CAS 1185315-17-6

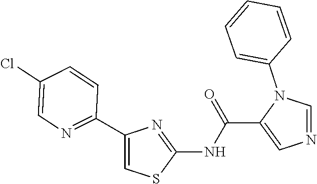 [SMILES: O=C(Nc1nc(-c2ccc(Cl)cn2)cs1)c1cncn1-c1ccccc1]

Acid CAS 135417-65-1
Amine CAS 1185315-17-6

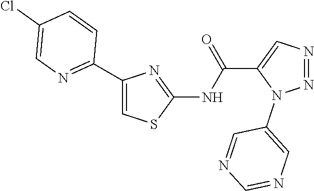 [SMILES: O=C(Nc1nc(-c2ccc(Cl)cn2)cs1)c1cnnn1-c1cncnc1]

Acid CAS 1478093-94-5
Amine CAS 1185315-17-6

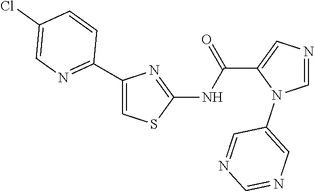 [SMILES: O=C(Nc1nc(-c2ccc(Cl)cn2)cs1)c1cncn1-c1cncnc1]

Acid CAS 1523297-19-9
Amine CAS 1185315-17-6

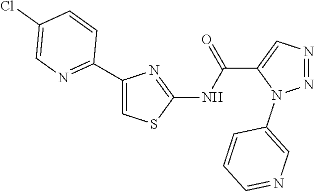 [SMILES: O=C(Nc1nc(-c2ccc(Cl)cn2)cs1)c1cnnn1-c1cccnc1]

Acid CAS 1476271-10-1
Amine CAS 1185315-17-6

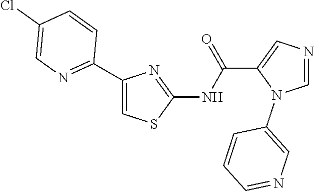 [SMILES: O=C(Nc1nc(-c2ccc(Cl)cn2)cs1)c1cncn1-c1cccnc1]

Acid CAS 1197398-67-6
Amine CAS 1185315-17-6

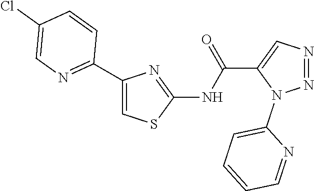 [SMILES: O=C(Nc1nc(-c2ccc(Cl)cn2)cs1)c1cnnn1-c1ccccn1]

Acid CAS 1480445-52-0
Amine CAS 1185315-17-6

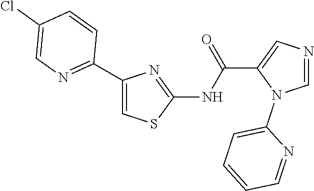 [SMILES: O=C(Nc1nc(-c2ccc(Cl)cn2)cs1)c1cncn1-c1ccccn1]

Acid CAS 1779128-15-2
Amine CAS 1185315-17-6

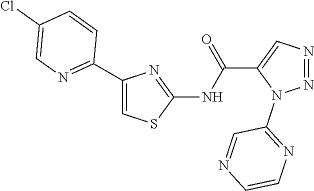 [SMILES: O=C(Nc1nc(-c2ccc(Cl)cn2)cs1)c1cnnn1-c1cnccn1]

Acid CAS 2946657-15-2
Amine CAS 1185315-17-6

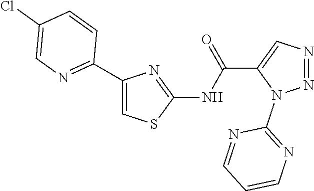 [SMILES: O=C(Nc1nc(-c2ccc(Cl)cn2)cs1)c1cnnn1-c1ncccn1]

Acid CAS 2946657-14-1
Amine CAS 1185315-17-6

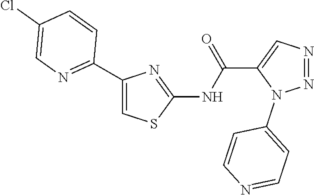 [SMILES: O=C(Nc1nc(-c2ccc(Cl)cn2)cs1)c1cnnn1-c1ccncc1]

Acid CAS 1525417-94-0
Amine CAS 1185315-17-6

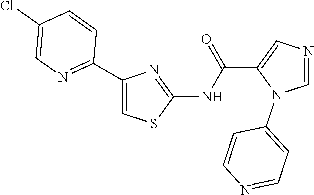 [SMILES: O=C(Nc1nc(-c2ccc(Cl)cn2)cs1)c1cncn1-c1ccncc1]

Acid CAS 1439900-49-8
Amine CAS 1185315-17-6

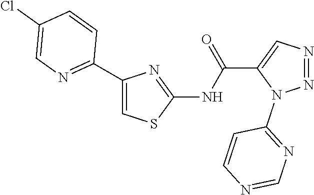 [SMILES: O=C(Nc1nc(-c2ccc(Cl)cn2)cs1)c1cnnn1-c1ccncn1]

Acid CAS 1482934-49-5
Amine CAS 1185315-17-6

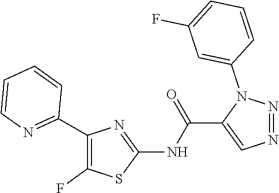 [SMILES: O=C(Nc1nc(-c2ccccn2)c(F)s1)c1cnnn1-c1cccc(F)c1]

Acid CAS 1153490-92-6
Amine CAS 1044270-93-0

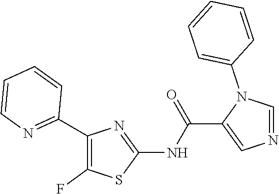 [SMILES: O=C(Nc1nc(-c2ccccn2)c(F)s1)c1cncn1-c1ccccc1]

Acid CAS 135417-65-1
Amine CAS 1044270-93-0

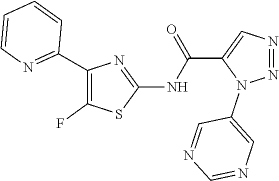 [SMILES: O=C(Nc1nc(-c2ccccn2)c(F)s1)c1cnnn1-c1cncnc1]

Acid CAS 1478093-94-5
Amine CAS 1044270-93-0

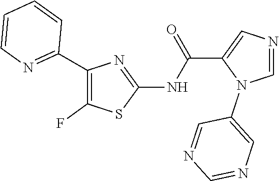 [SMILES: O=C(Nc1nc(-c2ccccn2)c(F)s1)c1cncn1-c1cncnc1]

Acid CAS 1523297-19-9
Amine CAS 1044270-93-0

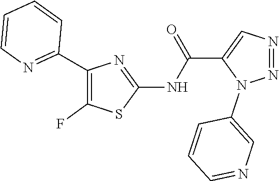 [SMILES: O=C(Nc1nc(-c2ccccn2)c(F)s1)c1cnnn1-c1cccnc1]

Acid CAS 1478271-10-1
Amine CAS 1044270-93-0

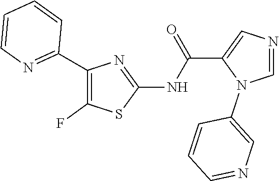 [SMILES: O=C(Nc1nc(-c2ccccn2)c(F)s1)c1cncn1-c1cccnc1]

Acid CAS 1197398-67-6
Amine CAS 1044270-93-0

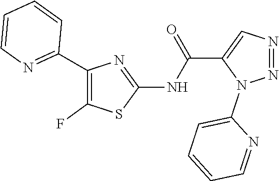 [SMILES: O=C(Nc1nc(-c2ccccn2)c(F)s1)c1cnnn1-c1ccccn1]

Acid CAS 1480445-52-0
Amine CAS 1044270-93-0

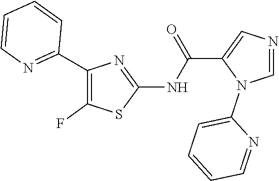 [SMILES: O=C(Nc1nc(-c2ccccn2)c(F)s1)c1cncn1-c1ccccn1]

Acid CAS 177912 [symbol] -15-2
Amine CAS 1044270-93-0

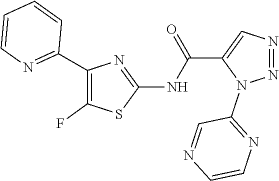 [SMILES: O=C(Nc1nc(-c2ccccn2)c(F)s1)c1cnnn1-c1cnccn1]

Acid CAS 2946657-15-2
Amine CAS 1044270-93-0

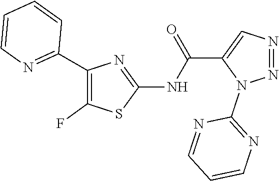 [SMILES: O=C(Nc1nc(-c2ccccn2)c(F)s1)c1cnnn1-c1ncccn1]

Acid CAS 2946657-14-1
Amine CAS 1044270-93-0

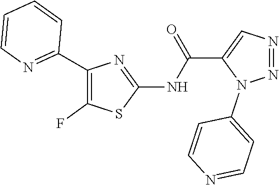 [SMILES: O=C(Nc1nc(-c2ccccn2)c(F)s1)c1cnnn1-c1ccncc1]

Acid CAS 1525417-94-0
Amine CAS 1044270-93-0

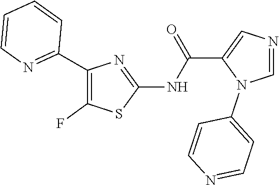 [SMILES: O=C(Nc1nc(-c2ccccn2)c(F)s1)c1cncn1-c1ccncc1]

Acid CAS 1439900-49-8
Amine CAS 1044270-93-0

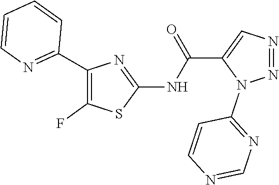 [SMILES: O=C(Nc1nc(-c2ccccn2)c(F)s1)c1cnnn1-c1ccncn1]

Acid CAS 1482934-49-5
Amine CAS 1044270-93-0

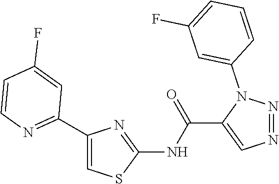 [SMILES: O=C(Nc1nc(-c2cc(F)ccn2)cs1)c1cnnn1-c1cccc(F)c1]

Acid CAS 1153490-92-6
Amine CAS 2228222-84-0

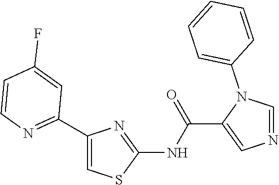 [SMILES: O=C(Nc1nc(-c2cc(F)ccn2)cs1)c1cncn1-c1ccccc1]

Acid CAS 135417-65-1
Amine CAS 2228222-84-0

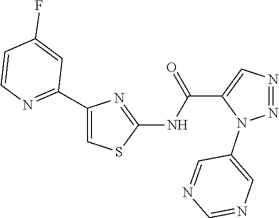 [SMILES: O=C(Nc1nc(-c2cc(F)ccn2)cs1)c1cnnn1-c1cncnc1]

Acid CAS 1478093-94-5
Amine CAS 2228222-84-0

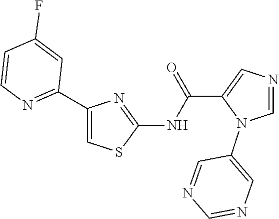 [SMILES: O=C(Nc1nc(-c2cc(F)ccn2)cs1)c1cncn1-c1cncnc1]

Acid CAS 1523297-19-9
Amine CAS 2228222-84-0

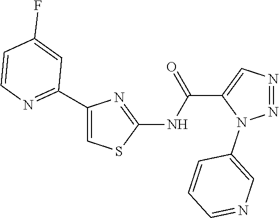 [SMILES: O=C(Nc1nc(-c2cc(F)ccn2)cs1)c1cnnn1-c1cccnc1]

Acid CAS 1478271-10-1
Amine CAS 2228222-84-0

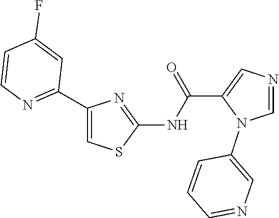 [SMILES: O=C(Nc1nc(-c2cc(F)ccn2)cs1)c1cncn1-c1cccnc1]

Acid CAS 119739 [symbol] -67-6
Amine CAS 2228222-84-0

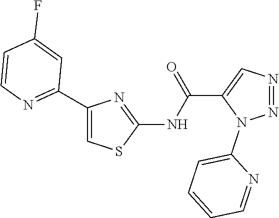 [SMILES: O=C(Nc1nc(-c2cc(F)ccn2)cs1)c1cnnn1-c1ccccn1]

Acid CAS 1480445-52-0
Amine CAS 2228222-84-0

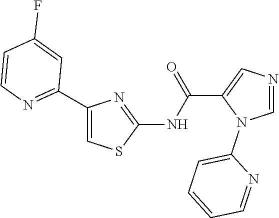 [SMILES: O=C(Nc1nc(-c2cc(F)ccn2)cs1)c1cncn1-c1ccccn1]

Acid CAS 1779128-15-2
Amine CAS 2228222-84-0

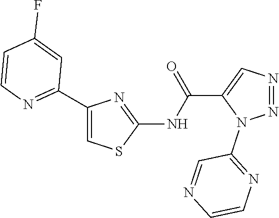 [SMILES: O=C(Nc1nc(-c2cc(F)ccn2)cs1)c1cnnn1-c1cnccn1]

Acid CAS 2946657-15-2
Amine CAS 2228222-84-0

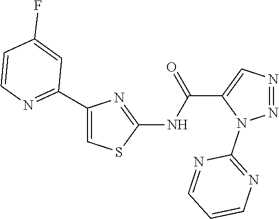 [SMILES: O=C(Nc1nc(-c2cc(F)ccn2)cs1)c1cnnn1-c1ncccn1]

Acid CAS 2946657-14-1
Amine CAS 2228222-84-0

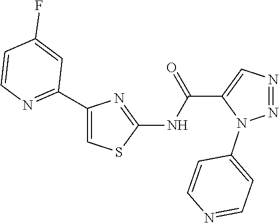 [SMILES: O=C(Nc1nc(-c2cc(F)ccn2)cs1)c1cnnn1-c1ccncc1]

Acid CAS 1525417-94-0
Amine CAS 2228222-84-0

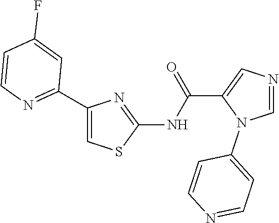 [SMILES: O=C(Nc1nc(-c2cc(F)ccn2)cs1)c1cncn1-c1ccncc1]

Acid CAS 1439900-49-8
Amine CAS 2228222-84-0

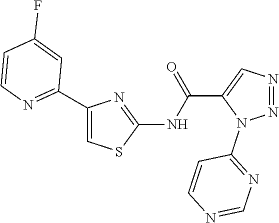 [SMILES: O=C(Nc1nc(-c2cc(F)ccn2)cs1)c1cnnn1-c1ccncn1]

Acid CAS 1482934-49-5
Amine CAS 2228222-84-0

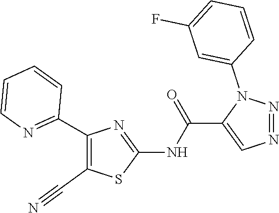 [SMILES: N#Cc1sc(NC(=O)c2cnnn2-c2cccc(F)c2)nc1-c1ccccn1]

Acid CAS 1153490-92-6
Amine CAS 1139743-06-8

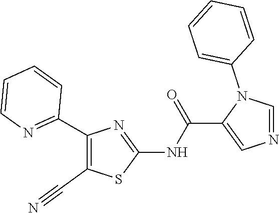 [SMILES: N#Cc1sc(NC(=O)c2cncn2-c2ccccc2)nc1-c1ccccn1]

Acid CAS 135417-65-1
Amine CAS 1139743-06-8

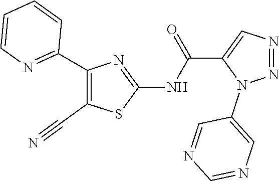 [SMILES: N#Cc1sc(NC(=O)c2cnnn2-c2cncnc2)nc1-c1ccccn1]

Acid CAS 1478093-94-5
Amine CAS 1139743-06-8

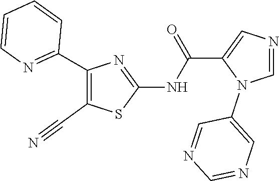 [SMILES: N#Cc1sc(NC(=O)c2cncn2-c2cncnc2)nc1-c1ccccn1]

Acid CAS 1523297-19-9
Amine CAS 1139743-06-8

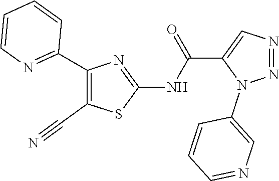 [SMILES: N#Cc1sc(NC(=O)c2cnnn2-c2cccnc2)nc1-c1ccccn1]

Acid CAS 1478271-10-1
Amine CAS 1139743-06-8

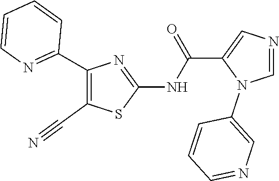 [SMILES: N#Cc1sc(NC(=O)c2cncn2-c2cccnc2)nc1-c1ccccn1]

Acid CAS 1197398-67-6
Amine CAS 1139743-06-8

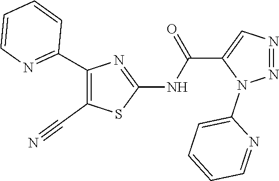 [SMILES: N#Cc1sc(NC(=O)c2cnnn2-c2ccccn2)nc1-c1ccccn1]

Acid CAS 1480445-52-0
Amine CAS 1139743-06-8

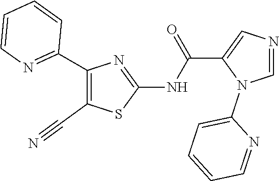 [SMILES: N#Cc1sc(NC(=O)c2cncn2-c2ccccn2)nc1-c1ccccn1]

Acid CAS 1779128-15-2
Amine CAS 1139743-06-8

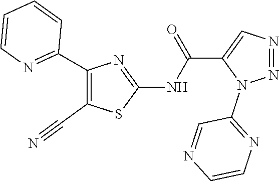 [SMILES: N#Cc1sc(NC(=O)c2cnnn2-c2cnccn2)nc1-c1ccccn1]

Acid CAS 2946657-15-2
Amine CAS 1139743-06-8

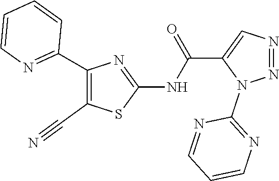 [SMILES: N#Cc1sc(NC(=O)c2cnnn2-c2ncccn2)nc1-c1ccccn1]

Acid CAS 2946657-14-1
Amine CAS 1139743-06-8

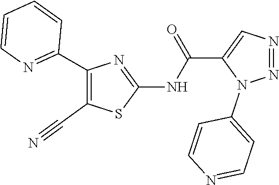 [SMILES: N#Cc1sc(NC(=O)c2cnnn2-c2ccncc2)nc1-c1ccccn1]

Acid CAS 1525417-94-0
Amine CAS 1139743-06-8

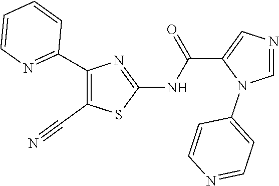 [SMILES: N#Cc1sc(NC(=O)c2cncn2-c2ccncc2)nc1-c1ccccn1]

Acid CAS 1439900-49-8
Amine CAS 1139743-06-8

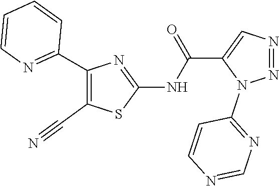 [SMILES: N#Cc1sc(NC(=O)c2cnnn2-c2ccncn2)nc1-c1ccccn1]

Acid CAS 1482934-49-5
Amine CAS 1139743-06-8

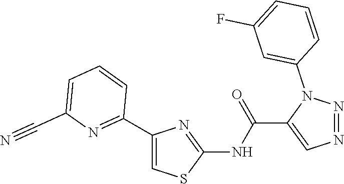 [SMILES: N#Cc1cccc(-c2csc(NC(=O)c3cnnn3-c3cccc(F)c3)n2)n1]

Acid CAS 1153490-92-6
Amine CAS 1185317-90-1

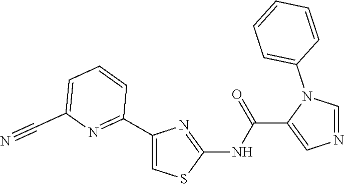 [SMILES: N#Cc1cccc(-c2csc(NC(=O)c3cncn3-c3ccccc3)n2)n1]

Acid CAS 135417-65-1
Amine CAS 1185317-90-1

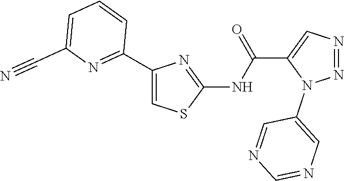 [SMILES: N#Cc1cccc(-c2csc(NC(=O)c3cnnn3-c3cncnc3)n2)n1]

Acid CAS 1478093-94-5
Amine CAS 1185317-90-1

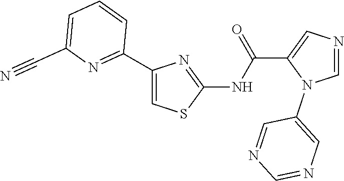 [SMILES: N#Cc1cccc(-c2csc(NC(=O)c3cncn3-c3cncnc3)n2)n1]

Acid CAS 1523297-19-9
Amine CAS 1185317-90-1

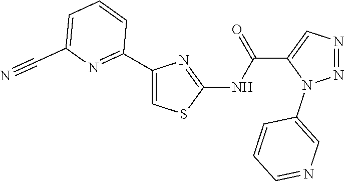 [SMILES: N#Cc1cccc(-c2csc(NC(=O)c3cnnn3-c3cccnc3)n2)n1]

Acid CAS 1478271-10-1
Amine CAS 1185317-90-1

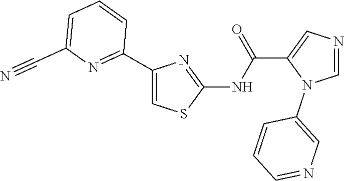 [SMILES: N#Cc1cccc(-c2csc(NC(=O)c3cncn3-c3cccnc3)n2)n1]

Acid CAS 1197398-67-6
Amine CAS 1185317-90-1

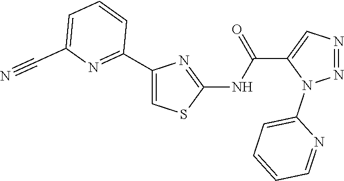 [SMILES: N#Cc1cccc(-c2csc(NC(=O)c3cnnn3-c3ccccn3)n2)n1]

Acid CAS 1480445-52-0
Amine CAS 1185317-90-1

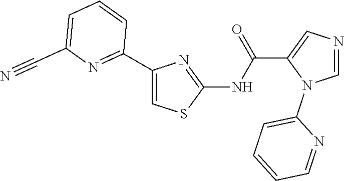 [SMILES: N#Cc1cccc(-c2csc(NC(=O)c3cncn3-c3ccccn3)n2)n1]

Acid CAS 1779128-15-2
Amine CAS 1185317-90-1

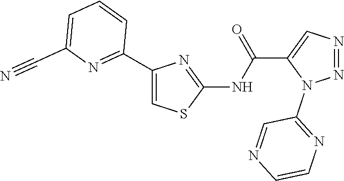 [SMILES: N#Cc1cccc(-c2csc(NC(=O)c3cnnn3-c3cnccn3)n2)n1]

Acid CAS 2946657-15-2
Amine CAS 1185317-90-1

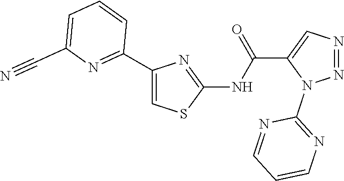 [SMILES: N#Cc1cccc(-c2csc(NC(=O)c3cnnn3-c3ncccn3)n2)n1]

Acid CAS 2946657-14-1
Amine CAS 1185317-90-1

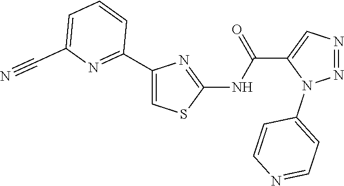 [SMILES: N#Cc1cccc(-c2csc(NC(=O)c3cnnn3-c3ccncc3)n2)n1]

Acid CAS 1525417-94-0
Amine CAS 1185317-90-1

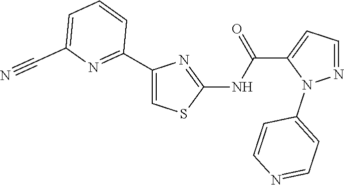 [SMILES: N#Cc1cccc(-c2csc(NC(=O)c3ccnn3-c3ccncc3)n2)n1]

Acid CAS 1439900-49-8
Amine CAS 1185317-90-1

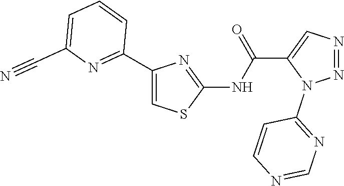 [SMILES: N#Cc1cccc(-c2csc(NC(=O)c3cnnn3-c3ccncn3)n2)n1]

Acid CAS 14 [symbol] 2934-49-5
Amine CAS 1185317-90-1

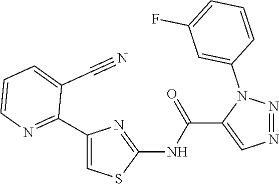 [SMILES: N#Cc1cccnc1-c1csc(NC(=O)c2cnnn2-c2cccc(F)c2)n1]

Acid CAS 1153490-92-6
Amine CAS 2253868-93-6

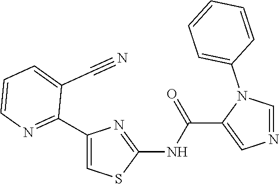 [SMILES: N#Cc1cccnc1-c1csc(NC(=O)c2cncn2-c2ccccc2)n1]

Acid CAS 135417-65-1
Amine CAS 2253868-93-6

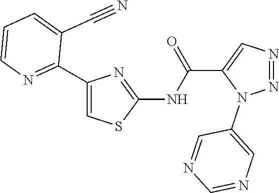 [SMILES: N#Cc1cccnc1-c1csc(NC(=O)c2cnnn2-c2cncnc2)n1]

Acid CAS 1478093-94-5
Amine CAS 2253868-93-6

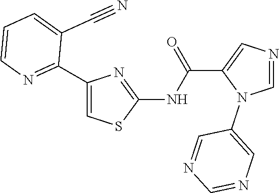 [SMILES: N#Cc1cccnc1-c1csc(NC(=O)c2cncn2-c2cncnc2)n1]

Acid CAS 1523297-19-9
Amine CAS 2253868-93-6

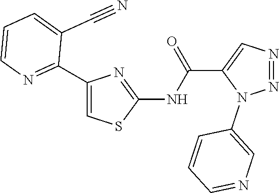 [SMILES: N#Cc1cccnc1-c1csc(NC(=O)c2cnnn2-c2cccnc2)n1]

Acid CAS 147827 [symbol] -10-1
Amine CAS 2253868-93-6

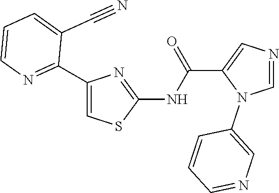 [SMILES: N#Cc1cccnc1-c1csc(NC(=O)c2cncn2-c2cccnc2)n1]

Acid CAS 1197398-67-6
Amine CAS 2253868-93-6

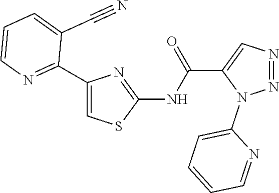 [SMILES: N#Cc1cccnc1-c1csc(NC(=O)c2cnnn2-c2ccccn2)n1]

Acid CAS 1480445-52-0
Amine CAS 2253868-93-6

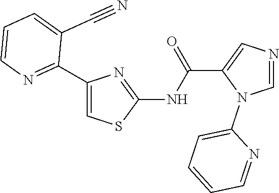 [SMILES: N#Cc1cccnc1-c1csc(NC(=O)c2cncn2-c2ccccn2)n1]

Acid CAS 1779128-15-2
Amine CAS 2253868-93-6

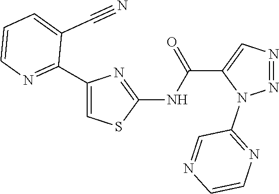 [SMILES: N#Cc1cccnc1-c1csc(NC(=O)c2cnnn2-c2cnccn2)n1]

Acid CAS 2946657-15-2
Amine CAS 2253868-93-6

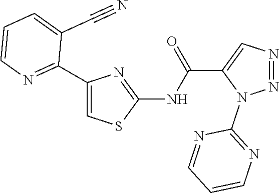 [SMILES: N#Cc1cccnc1-c1csc(NC(=O)c2cnnn2-c2ncccn2)n1]

Acid CAS 2946657-14-1
Amine CAS 2253868-93-6

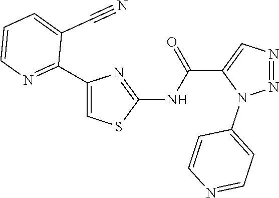 [SMILES: N#Cc1cccnc1-c1csc(NC(=O)c2cnnn2-c2ccncc2)n1]

Acid CAS 1525417-94-0
Amine CAS 2253868-93-6

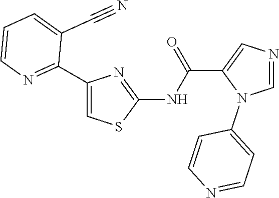 [SMILES: N#Cc1cccnc1-c1csc(NC(=O)c2cncn2-c2ccncc2)n1]

Acid CAS 1439900-499- [symbol]
Amine CAS 2253868-93-6

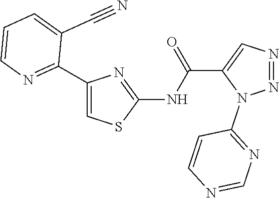 [SMILES: N#Cc1cccnc1-c1csc(NC(=O)c2cnnn2-c2ccncn2)n1]

Acid CAS 14 [symbol] 2934-49-5
Amine CAS 2253868-93-6

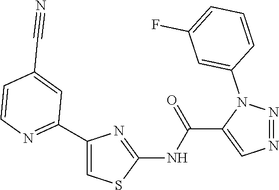 [SMILES: N#Cc1ccnc(-c2csc(NC(=O)c3cnnn3-c3cccc(F)c3)n2)c1]

Acid CAS 1153490-92-6
Amine CAS 2253868-97-0

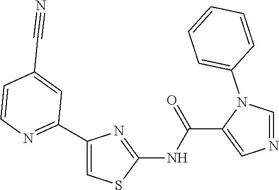 [SMILES: N#Cc1ccnc(-c2csc(NC(=O)c3cncn3-c3ccccc3)n2)c1]

Acid CAS 135417-65-1
Amine CAS 2253868-97-0

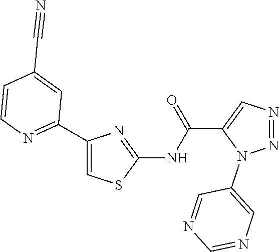 [SMILES: N#Cc1ccnc(-c2csc(NC(=O)c3cnnn3-c3cncnc3)n2)c1]

Acid CAS 1478093-94-5
Amine CAS 2253868-97-0

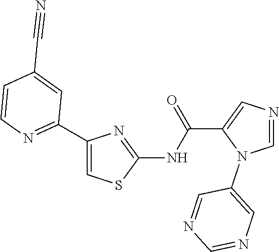 [SMILES: N#Cc1ccnc(-c2csc(NC(=O)c3cncn3-c3cncnc3)n2)c1]

Acid CAS 1523297-19-9
Amine CAS 2253868-97-0

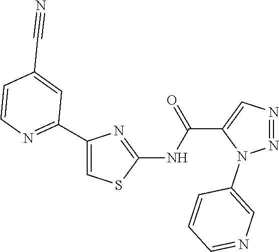 [SMILES: N#Cc1ccnc(-c2csc(NC(=O)c3cnnn3-c3cccnc3)n2)c1]

Acid CAS 147 [symbol] 271-10-1
Amine CAS 2253868-97-0

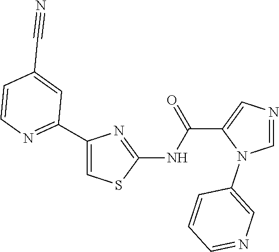 [SMILES: N#Cc1ccnc(-c2csc(NC(=O)c3cncn3-c3cccnc3)n2)c1]

Acid CAS 1197398-67-6
Amine CAS 2253868-97-0

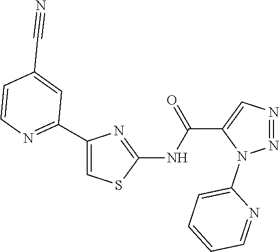 [SMILES: N#Cc1ccnc(-c2csc(NC(=O)c3cnnn3-c3ccccn3)n2)c1]

Acid CAS 1480445-52-0
Amine CAS 2253868-97-0

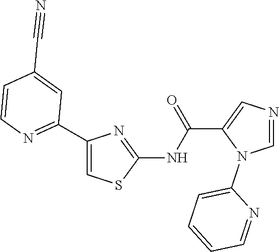 [SMILES: N#Cc1ccnc(-c2csc(NC(=O)c3cncn3-c3ccccn3)n2)c1]

Acid CAS 1779128-15-2
Amine CAS 2253868-97-0

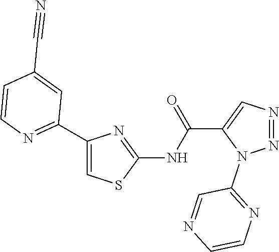 [SMILES: N#Cc1ccnc(-c2csc(NC(=O)c3cnnn3-c3cnccn3)n2)c1]

Acid CAS 2946657-15-2
Amine CAS 2253868-97-0

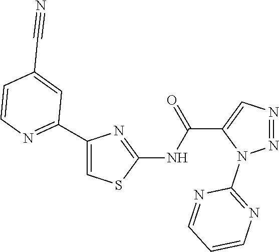 [SMILES: N#Cc1ccnc(-c2csc(NC(=O)c3cnnn3-c3ncccn3)n2)c1]

Acid CAS 2946657-14-1
Amine CAS 2253868-97-0

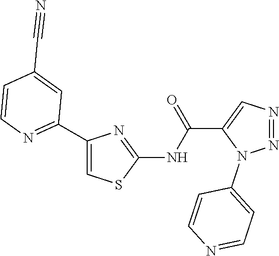 [SMILES: N#Cc1ccnc(-c2csc(NC(=O)c3cnnn3-c3ccncc3)n2)c1]

Acid CAS 1525417-94-0
Amine CAS 2253868-97-0

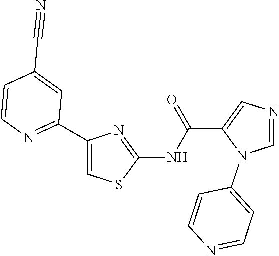 [SMILES: N#Cc1ccnc(-c2csc(NC(=O)c3cncn3-c3ccncc3)n2)c1]

Acid CAS 1439900-49-8
Amine CAS 2253868-97-0

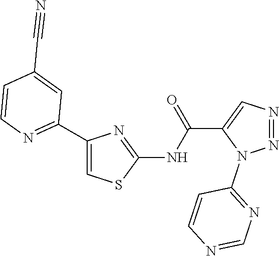 [SMILES: N#Cc1ccnc(-c2csc(NC(=O)c3cnnn3-c3ccncn3)n2)c1]

Acid CAS 1482934-49-5
Amine CAS 2253868-97-0

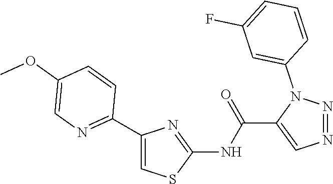 [SMILES: COc1ccc(-c2csc(NC(=O)c3cnnn3-c3cccc(F)c3)n2)nc1]

Acid CAS 1153490-92-6
Amine CAS 2228346-85-6

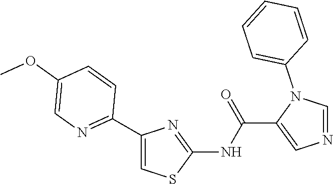 [SMILES: COc1ccc(-c2csc(NC(=O)c3cncn3-c3ccccc3)n2)nc1]

Acid CAS 135417-65-1
Amine CAS 2228346-85-6

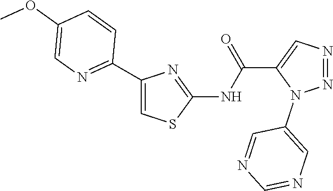 [SMILES: COc1ccc(-c2csc(NC(=O)c3cnnn3-c3cncnc3)n2)nc1]

Acid CAS 1478093-94-5
Amine CAS 2228346-65-6

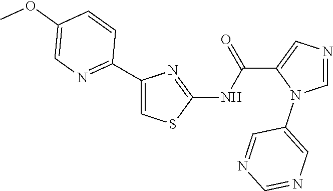 [SMILES: COc1ccc(-c2csc(NC(=O)c3cncn3-c3cncnc3)n2)nc1]

Acid CAS 1523297-19-9
Amine CAS 2226346-85-6

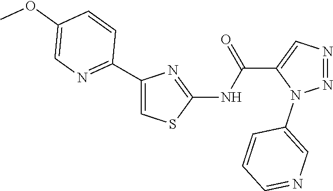 [SMILES: COc1ccc(-c2csc(NC(=O)c3cnnn3-c3cccnc3)n2)nc1]

Acid CAS 1476271-10-1
Amine CAS 2228346-85-6

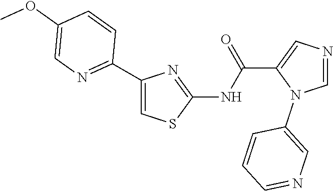 [SMILES: COc1ccc(-c2csc(NC(=O)c3cncn3-c3cccnc3)n2)nc1]

Acid CAS 1197396-67-6
Amine CAS 2228346-85-6

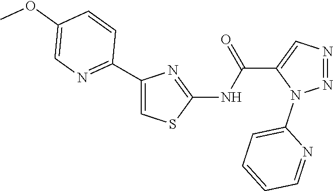 [SMILES: COc1ccc(-c2csc(NC(=O)c3cnnn3-c3ccccn3)n2)nc1]

Acid CAS 14 [symbol] 0445-52-0
Amine CAS 2228346-85-6

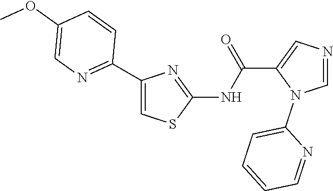 [SMILES: COc1ccc(-c2csc(NC(=O)c3cncn3-c3ccccn3)n2)nc1]

Acid CAS 1779128-15-2
Amine CAS 2226346-85-6

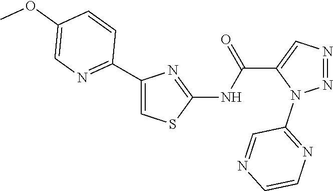 [SMILES: COc1ccc(-c2csc(NC(=O)c3cnnn3-c3cnccn3)n2)nc1]

Acid CAS 2946657-15-2
Amine CAS 2228346-85-6

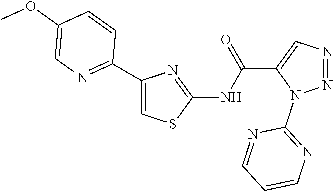 [SMILES: COc1ccc(-c2csc(NC(=O)c3cnnn3-c3ncccn3)n2)nc1]

Acid CAS 2946657-14-1
Amine CAS 2228346-85-6

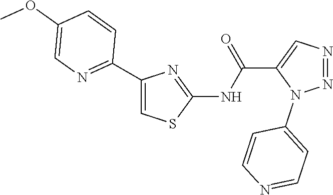 [SMILES: COc1ccc(-c2csc(NC(=O)c3cnnn3-c3ccncc3)n2)nc1]

Acid CAS 1525417-94-0
Amine CAS 2228346-85-6

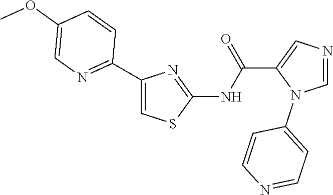 [SMILES: COc1ccc(-c2csc(NC(=O)c3cncn3-c3ccncc3)n2)nc1]

Acid CAS 1439900-49-8
Amine CAS 2228346-85-6

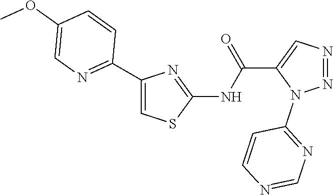 [SMILES: COc1ccc(-c2csc(NC(=O)c3cnnn3-c3ccncn3)n2)nc1]

Acid CAS 1482934-49-5
Amine CAS 2228346-85-6

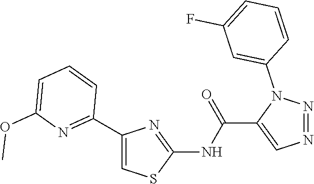 [SMILES: COc1cccc(-c2csc(NC(=O)c3cnnn3-c3cccc(F)c3)n2)n1]

Acid CAS 1153490-92-6
Amine CAS 1 [symbol] 70028-77-5

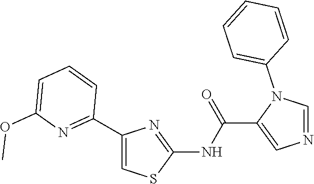 [SMILES: COc1cccc(-c2csc(NC(=O)c3cncn3-c3ccccc3)n2)n1]

Acid CAS 135417-65-1
Amine CAS 1 [symbol] 70028-77-5

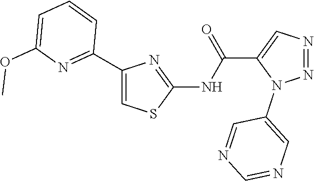 [SMILES: COc1cccc(-c2csc(NC(=O)c3cnnn3-c3cncnc3)n2)n1]

Acid CAS 1478093-94-5
Amine CAS 1 [symbol] 70028-77-5

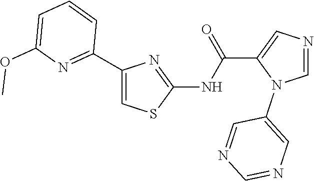 [SMILES: COc1cccc(-c2csc(NC(=O)c3cncn3-c3cncnc3)n2)n1]

Acid CAS 1523297-19-9
Amine CAS 1 [symbol] 70028-77-5

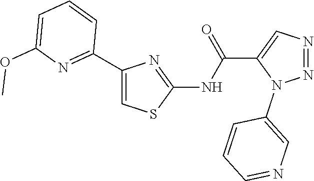 [SMILES: COc1cccc(-c2csc(NC(=O)c3cnnn3-c3cccnc3)n2)n1]

Acid CAS 1476271-10-1
Amine CAS 1 [symbol] 70028-77-5

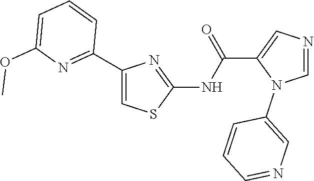 [SMILES: COc1cccc(-c2csc(NC(=O)c3cncn3-c3cccnc3)n2)n1]

Acid CAS 1197398-67-6
Amine CAS 1 [symbol] 70028-77-5

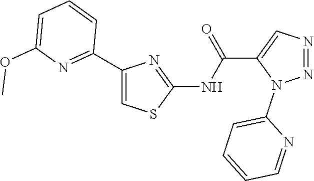 [SMILES: COc1cccc(-c2csc(NC(=O)c3cnnn3-c3ccccn3)n2)n1]

Acid CAS 1480445-52-0
Amine CAS 1 [symbol] 70028-77-5

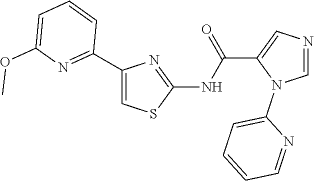 [SMILES: COc1cccc(-c2csc(NC(=O)c3cncn3-c3ccccn3)n2)n1]

Acid CAS 1779128-15-2
Amine CAS 1 [symbol] 70028-77-5

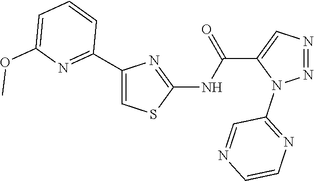 [SMILES: COc1cccc(-c2csc(NC(=O)c3cnnn3-c3cnccn3)n2)n1]

Acid CAS 2946657-15-2
Amine CAS 1 [symbol] 70028-77-5

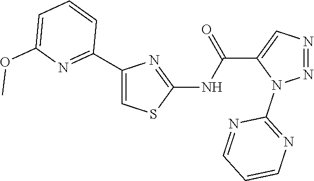 [SMILES: COc1cccc(-c2csc(NC(=O)c3cnnn3-c3ncccn3)n2)n1]

Acid CAS 2946657-15-2
Amine CAS 1 [symbol] 70028-77-5

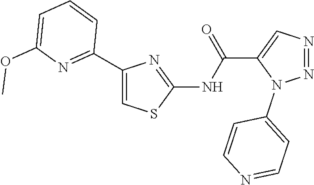 [SMILES: COc1cccc(-c2csc(NC(=O)c3cnnn3-c3ccncc3)n2)n1]

Acid CAS 1525417-94-0
Amine CAS 1870028-77-5

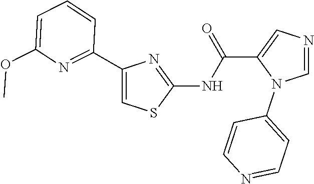 [SMILES: COc1cccc(-c2csc(NC(=O)c3cncn3-c3ccncc3)n2)n1]

Acid CAS 1439900-49-8
Amine CAS 1 [symbol] 70028-77-5

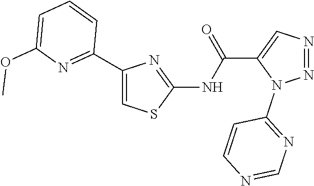 [SMILES: COc1cccc(-c2csc(NC(=O)c3cnnn3-c3ccncn3)n2)n1]

Acid CAS 14 [symbol] 2934-49-5
Amine CAS 1 [symbol] 70028-77-5

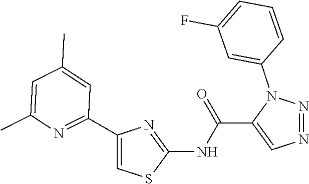 [SMILES: Cc1cc(C)nc(-c2csc(NC(=O)c3cnnn3-c3cccc(F)c3)n2)c1]

Acid CAS 1153490-92-6
Amine CAS 1865056-88-7

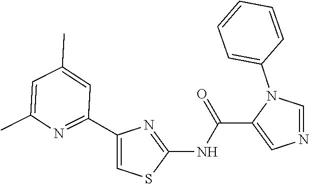 [SMILES: Cc1cc(C)nc(-c2csc(NC(=O)c3cncn3-c3ccccc3)n2)c1]

Acid CAS 135417-65-1
Amine CAS 1865056-88-7

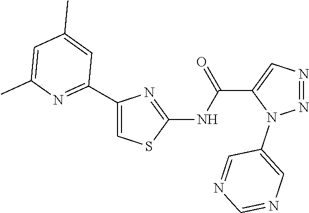 [SMILES: Cc1cc(C)nc(-c2csc(NC(=O)c3cnnn3-c3cncnc3)n2)c1]

Acid CAS 1478093-94-5
Amine CAS 1865056-88-7

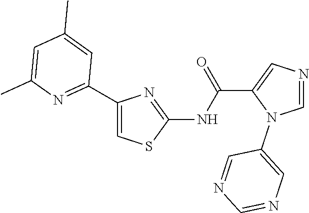 [SMILES: Cc1cc(C)nc(-c2csc(NC(=O)c3cncn3-c3cncnc3)n2)c1]

Acid CAS 1523297-19-9
Amine CAS 1865056-88-7

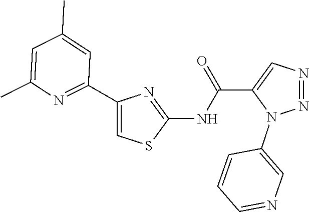 [SMILES: Cc1cc(C)nc(-c2csc(NC(=O)c3cnnn3-c3cccnc3)n2)c1]

Acid CAS 1478271-10-1
Amine CAS 1865056-88-7

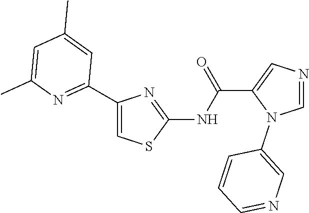 [SMILES: Cc1cc(C)nc(-c2csc(NC(=O)c3cncn3-c3cccnc3)n2)c1]

Acid CAS 1197398-67-6
Amine CAS 1865056-88-7

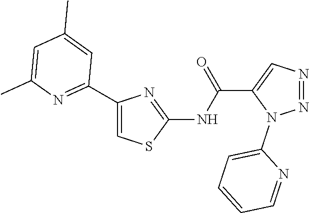 [SMILES: Cc1cc(C)nc(-c2csc(NC(=O)c3cnnn3-c3ccccn3)n2)c1]

Acid CAS 1480445-52-0
Amine CAS 1865056-88-7

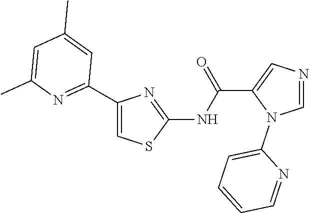 [SMILES: Cc1cc(C)nc(-c2csc(NC(=O)c3cncn3-c3ccccn3)n2)c1]

Acid CAS 1779128-15-2
Amine CAS 1865056-88-7

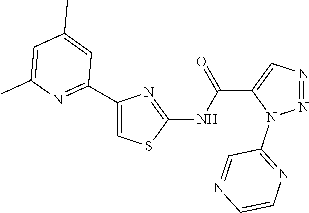 [SMILES: Cc1cc(C)nc(-c2csc(NC(=O)c3cnnn3-c3cnccn3)n2)c1]

Acid CAS 2946657-15-2
Amine CAS 1865056-88-7

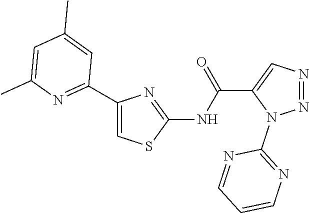 [SMILES: Cc1cc(C)nc(-c2csc(NC(=O)c3cnnn3-c3ncccn3)n2)c1]

Acid CAS 2946657-14-1
Amine CAS 1865056-88-7

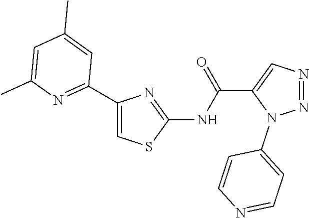 [SMILES: Cc1cc(C)nc(-c2csc(NC(=O)c3cnnn3-c3ccncc3)n2)c1]

Acid CAS 1525417-94-0
Amine CAS 1865056-88-7

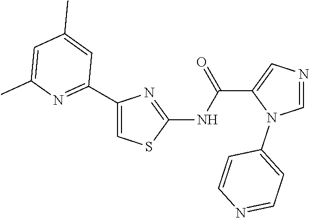 [SMILES: Cc1cc(C)nc(-c2csc(NC(=O)c3cncn3-c3ccncc3)n2)c1]

Acid CAS 1439900-49-8
Amine CAS 1865056-88-7

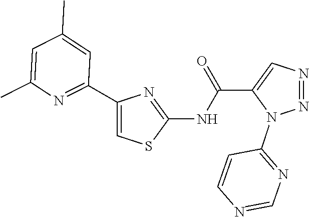 [SMILES: Cc1cc(C)nc(-c2csc(NC(=O)c3cnnn3-c3ccncn3)n2)c1]

Acid CAS 1482934-49-5
Amine CAS 1865056-88-7

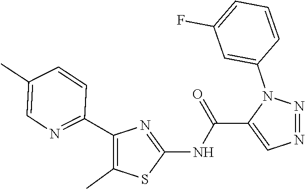 [SMILES: Cc1ccc(-c2nc(NC(=O)c3cnnn3-c3cccc(F)c3)sc2C)nc1]

Acid CAS 1153490-92-6
Amine CAS 2294596-48-6

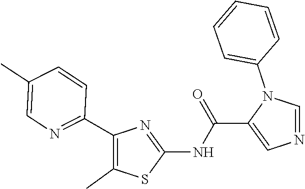 [SMILES: Cc1ccc(-c2nc(NC(=O)c3cncn3-c3ccccc3)sc2C)nc1]

Acid CAS 135417-65-1
Amine CAS 2294596-48-6

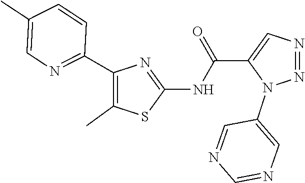 [SMILES: Cc1ccc(-c2nc(NC(=O)c3cnnn3-c3cncnc3)sc2C)nc1]

Acid CAS 1478093-94-5
Amine CAS 2294596-48-6

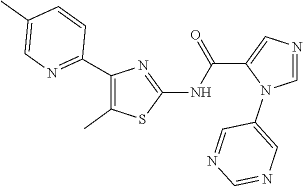 [SMILES: Cc1ccc(-c2nc(NC(=O)c3cncn3-c3cncnc3)sc2C)nc1]

Acid CAS 1523297-19-9
Amine CAS 2294596-48-6

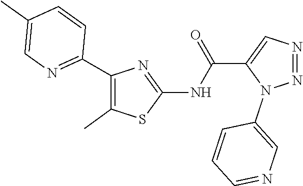 [SMILES: Cc1ccc(-c2nc(NC(=O)c3cnnn3-c3cccnc3)sc2C)nc1]

Acid CAS 1478271-10-1
Amine CAS 2294596-48-6

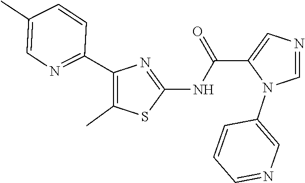 [SMILES: Cc1ccc(-c2nc(NC(=O)c3cncn3-c3cccnc3)sc2C)nc1]

Acid CAS 1197398-67-6
Amine CAS 2294596-48-6

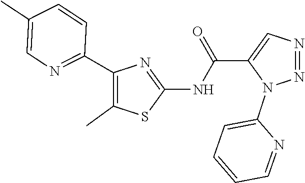 [SMILES: Cc1ccc(-c2nc(NC(=O)c3cnnn3-c3ccccn3)sc2C)nc1]

Acid CAS 1480445-52-0
Amine CAS 2294596-48-6

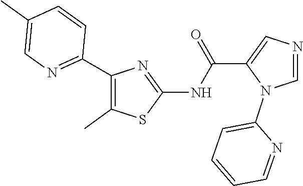 [SMILES: Cc1ccc(-c2nc(NC(=O)c3cncn3-c3ccccn3)sc2C)nc1]

Acid CAS 1779128-15-2
Amine CAS 2294596-48-6

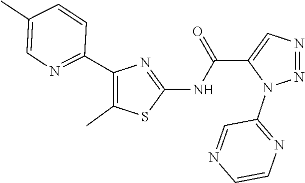 [SMILES: Cc1ccc(-c2nc(NC(=O)c3cnnn3-c3cnccn3)sc2C)nc1]

Acid CAS 2946657-15-2
Amine CAS 2294596-48-6

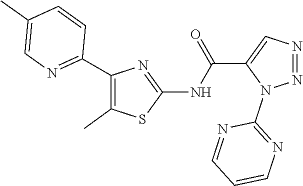 [SMILES: Cc1ccc(-c2nc(NC(=O)c3cnnn3-c3ncccn3)sc2C)nc1]

Acid CAS 2946657-14-1
Amine CAS 2294596-48-6

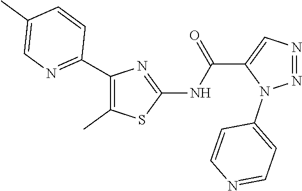 [SMILES: Cc1ccc(-c2nc(NC(=O)c3cnnn3-c3ccncc3)sc2C)nc1]

Acid CAS 1525417-94-0
Amine CAS 2294596-48-6

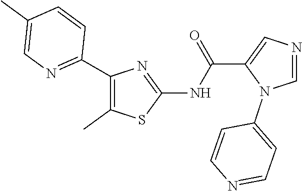 [SMILES: Cc1ccc(-c2nc(NC(=O)c3cncn3-c3ccncc3)sc2C)nc1]

Acid CAS 1439900-49-8
Amine CAS 2294598-48-6

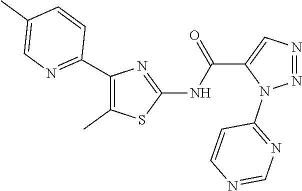 [SMILES: Cc1ccc(-c2nc(NC(=O)c3cnnn3-c3ccncn3)sc2C)nc1]

Acid CAS 1482934-49-5
Amine CAS 2294596-48-6

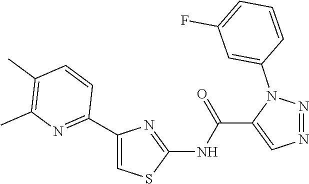 [SMILES: Cc1ccc(-c2csc(NC(=O)c3cnnn3-c3cccc(F)c3)n2)nc1C]

Acid CAS 1153490-92-6
Amine CAS 1934459-93-4

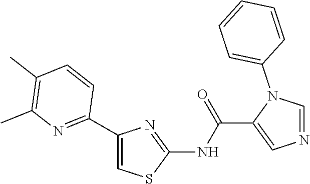 [SMILES: Cc1ccc(-c2csc(NC(=O)c3cncn3-c3ccccc3)n2)nc1C]

Acid CAS 135417-65-1
Amine CAS 1934459-93-4

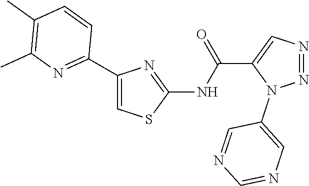 [SMILES: Cc1ccc(-c2csc(NC(=O)c3cnnn3-c3cncnc3)n2)nc1C]

Acid CAS 1478093-94-5
Amine CAS 1934459-93-4

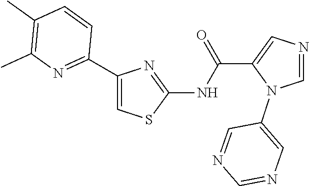 [SMILES: Cc1ccc(-c2csc(NC(=O)c3cncn3-c3cncnc3)n2)nc1C]

Acid CAS 1523297-19-9
Amine CAS 1934459-93-4

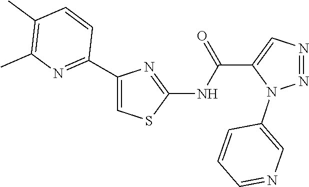 [SMILES: Cc1ccc(-c2csc(NC(=O)c3cnnn3-c3cccnc3)n2)nc1C]

Acid CAS 1478271-10-1
Amine CAS 1934459-93-4

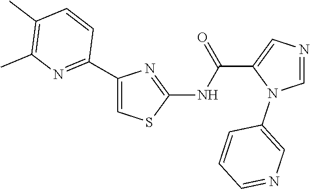 [SMILES: Cc1ccc(-c2csc(NC(=O)c3cncn3-c3cccnc3)n2)nc1C]

Acid CAS 1197396-67-6
Amine CAS 1934459-93-4

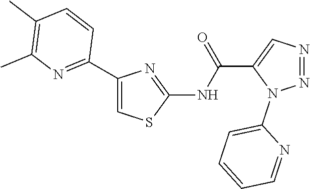 [SMILES: Cc1ccc(-c2csc(NC(=O)c3cnnn3-c3ccccn3)n2)nc1C]

Acid CAS 1460445-52-0
Amine CAS 1934459-93-4

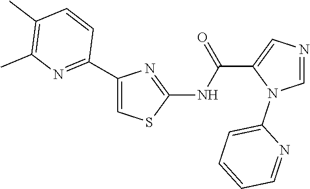 [SMILES: Cc1ccc(-c2csc(NC(=O)c3cncn3-c3ccccn3)n2)nc1C]

Acid CAS 177912 [symbol] -15-2
Amine CAS 1934459-93-4

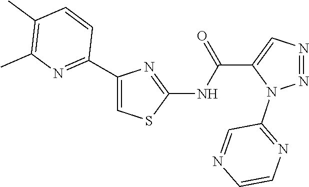 [SMILES: Cc1ccc(-c2csc(NC(=O)c3cnnn3-c3cnccn3)n2)nc1C]

Acid CAS 2946657-15-2
Amine CAS 1934459-93-4

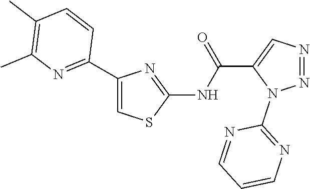 [SMILES: Cc1ccc(-c2csc(NC(=O)c3cnnn3-c3ncccn3)n2)nc1C]

Acid CAS 2946657-14-1
Amine CAS 1934459-93-4

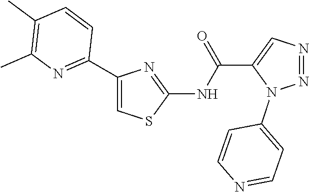 [SMILES: Cc1ccc(-c2csc(NC(=O)c3cnnn3-c3ccncc3)n2)nc1C]

Acid CAS 1525417-94-0
Amine CAS 1934459-93-4

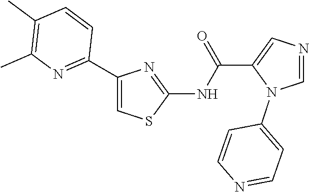 [SMILES: Cc1ccc(-c2csc(NC(=O)c3cncn3-c3ccncc3)n2)nc1C]

Acid CAS 1439900-49-8
Amine CAS 1934459-93-4

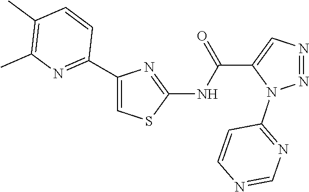 [SMILES: Cc1ccc(-c2csc(NC(=O)c3cnnn3-c3ccncn3)n2)nc1C]

Acid CAS 14 [symbol] 2934-49-5
Amine CAS 1934459-93-4

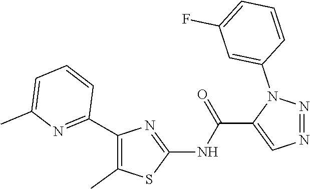 [SMILES: Cc1cccc(-c2nc(NC(=O)c3cnnn3-c3cccc(F)c3)sc2C)n1]

Acid CAS 1153490-92-6
Amine CAS 2169472-77-7

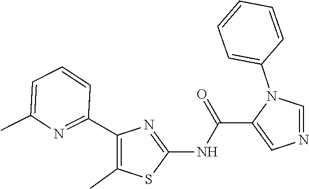 [SMILES: Cc1cccc(-c2nc(NC(=O)c3cncn3-c3ccccc3)sc2C)n1]

Acid CAS 135417-65-1
Amine CAS 2169472-77-7

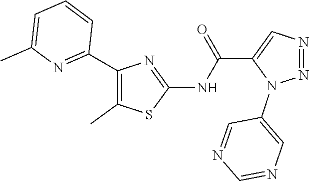 [SMILES: Cc1cccc(-c2nc(NC(=O)c3cnnn3-c3cncnc3)sc2C)n1]

Acid CAS 147 [symbol] 093-94-5
Amine CAS 2169472-77-7

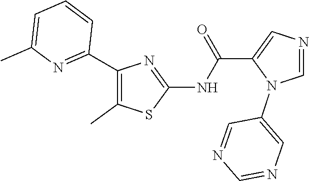 [SMILES: Cc1cccc(-c2nc(NC(=O)c3cncn3-c3cncnc3)sc2C)n1]

Acid CAS 1523297-19-9
Amine CAS 2169472-77-7

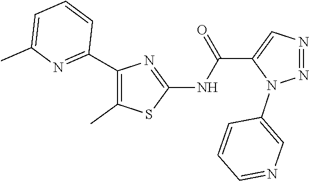 [SMILES: Cc1cccc(-c2nc(NC(=O)c3cnnn3-c3cccnc3)sc2C)n1]

Acid CAS 147 [symbol] 271-10-1
Amine CAS 2169472-77-7

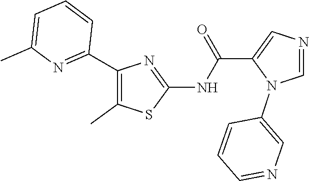 [SMILES: Cc1cccc(-c2nc(NC(=O)c3cncn3-c3cccnc3)sc2C)n1]

Acid CAS 1197398-67-6
Amine CAS 2169472-77-7

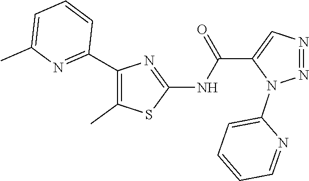 [SMILES: Cc1cccc(-c2nc(NC(=O)c3cnnn3-c3ccccn3)sc2C)n1]

Acid CAS 1480445-52-0
Amine CAS 2169472-77-7

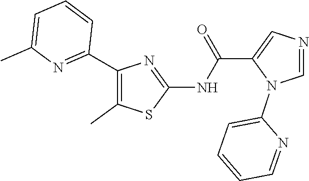 [SMILES: Cc1cccc(-c2nc(NC(=O)c3cncn3-c3ccccn3)sc2C)n1]

Acid CAS 1779128-15-2
Amine CAS 2169472-77-7

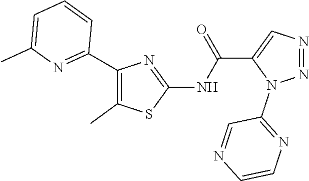 [SMILES: Cc1cccc(-c2nc(NC(=O)c3cnnn3-c3cnccn3)sc2C)n1]

Acid CAS 2946657-15-2
Amine CAS 2169472-77-7

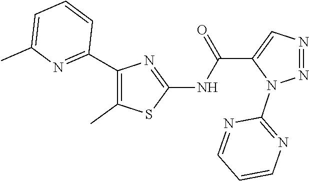 [SMILES: Cc1cccc(-c2nc(NC(=O)c3cnnn3-c3ncccn3)sc2C)n1]

Acid CAS 2946657-14-1
Amine CAS 2169472-77-7

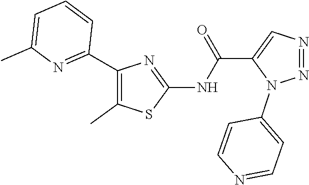 [SMILES: Cc1cccc(-c2nc(NC(=O)c3cnnn3-c3ccncc3)sc2C)n1]

Acid CAS 1525417-94-0
Amine CAS 2169472-77-7

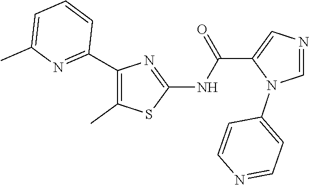 [SMILES: Cc1cccc(-c2nc(NC(=O)c3cncn3-c3ccncc3)sc2C)n1]

Acid CAS 1439900-49-6
Amine CAS 2169472-77-7

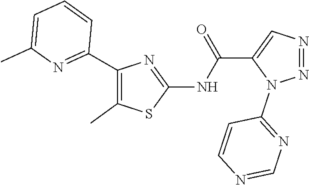 [SMILES: Cc1cccc(-c2nc(NC(=O)c3cnnn3-c3ccncn3)sc2C)n1]

Acid CAS 14829 [symbol] 4-49-5
Amine CAS 2169472-77-7

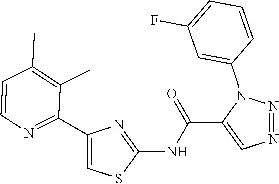 [SMILES: Cc1ccnc(-c2csc(NC(=O)c3cnnn3-c3cccc(F)c3)n2)c1C]

Acid CAS 1153490-92-6
Amine CAS 2169166-95-2

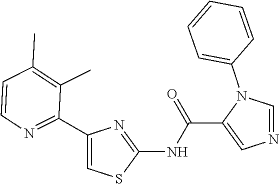 [SMILES: Cc1ccnc(-c2csc(NC(=O)c3cncn3-c3ccccc3)n2)c1C]

Acid CAS 135417-65-1
Amine CAS 2169166-95-2

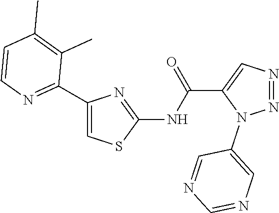 [SMILES: Cc1ccnc(-c2csc(NC(=O)c3cnnn3-c3cncnc3)n2)c1C]

Acid CAS 147 [symbol] 093-94-5
Amine CAS 2169166-95-2

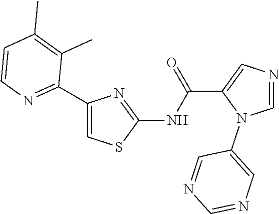 [SMILES: Cc1ccnc(-c2csc(NC(=O)c3cncn3-c3cncnc3)n2)c1C]

Acid CAS 1523297-19-9
Amine CAS 2169166-95-2

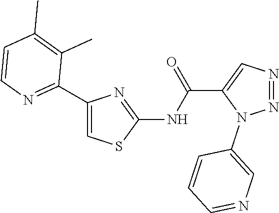 [SMILES: Cc1ccnc(-c2csc(NC(=O)c3cnnn3-c3cccnc3)n2)c1C]

Acid CAS 1478271-10-1
Amine CAS 2169166-95-2

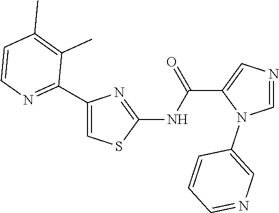 [SMILES: Cc1ccnc(-c2csc(NC(=O)c3cncn3-c3cccnc3)n2)c1C]

Acid CAS 119739 [symbol] -67-6
Amine CAS 2169166-95-2

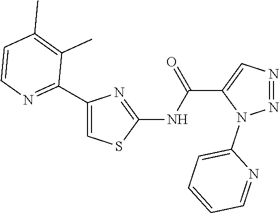 [SMILES: Cc1ccnc(-c2csc(NC(=O)c3cnnn3-c3ccccn3)n2)c1C]

Acid CAS 14 [symbol] 0445-52-0
Amine CAS 2169166-95-2

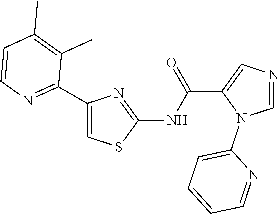 [SMILES: Cc1ccnc(-c2csc(NC(=O)c3cncn3-c3ccccn3)n2)c1C]

Acid CAS 1779128-15-2
Amine CAS 2169166-95-2

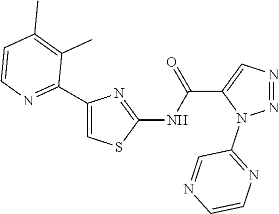 [SMILES: Cc1ccnc(-c2csc(NC(=O)c3cnnn3-c3cnccn3)n2)c1C]

Acid CAS 2946657-15-2
Amine CAS 2169166-95-2

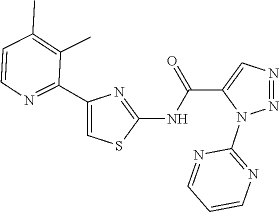 [SMILES: Cc1ccnc(-c2csc(NC(=O)c3cnnn3-c3ncccn3)n2)c1C]

Acid CAS 2946657-14-1
Amine CAS 2169166-95-2

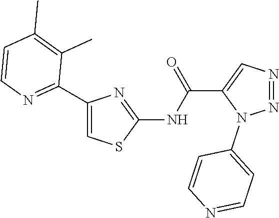 [SMILES: Cc1ccnc(-c2csc(NC(=O)c3cnnn3-c3ccncc3)n2)c1C]

Acid CAS 1525417-94-0
Amine CAS 2169166-95-2

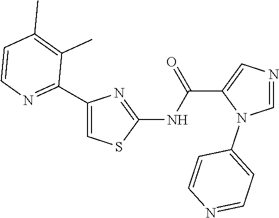 [SMILES: Cc1ccnc(-c2csc(NC(=O)c3cncn3-c3ccncc3)n2)c1C]

Acid CAS 1439900-49-8
Amine CAS 2169166-95-2

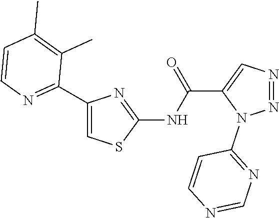 [SMILES: Cc1ccnc(-c2csc(NC(=O)c3cnnn3-c3ccncn3)n2)c1C]

Acid CAS 14 [symbol] 2934-49-5
Amine CAS 2169166-95-2

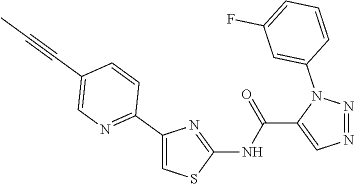 [SMILES: CC#Cc1ccc(-c2csc(NC(=O)c3cnnn3-c3cccc(F)c3)n2)nc1]

Acid CAS 1153490-92-6
Amine CAS 2592824-00-3

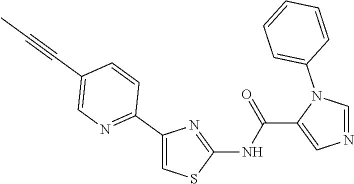 [SMILES: CC#Cc1ccc(-c2csc(NC(=O)c3cncn3-c3ccccc3)n2)nc1]

Acid CAS 135417-65-1
Amine CAS 2592824-00-3

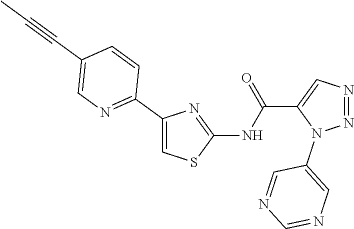 [SMILES: CC#Cc1ccc(-c2csc(NC(=O)c3cnnn3-c3cncnc3)n2)nc1]

Acid CAS 1476093-94-5
Amine CAS 2592824-00-3

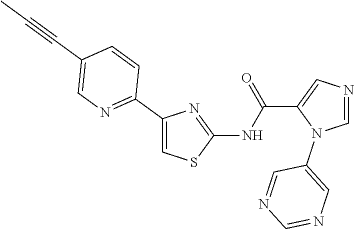 [SMILES: CC#Cc1ccc(-c2csc(NC(=O)c3cncn3-c3cncnc3)n2)nc1]

Acid CAS 1523297-19-9
Amine CAS 2592824-00-3

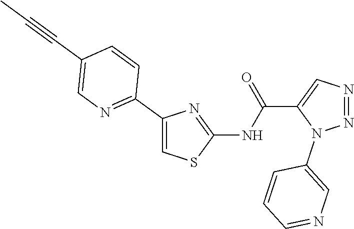 [SMILES: CC#Cc1ccc(-c2csc(NC(=O)c3cnnn3-c3cccnc3)n2)nc1]

Acid CAS 147 [symbol] 271-10-1
Amine CAS 2592824-00-3

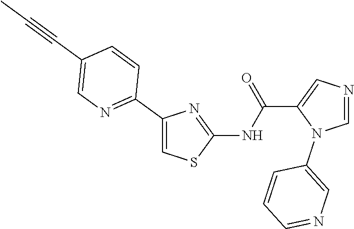 [SMILES: CC#Cc1ccc(-c2csc(NC(=O)c3cncn3-c3cccnc3)n2)nc1]

Acid CAS 1197398-67-6
Amine CAS 2592824-00-3

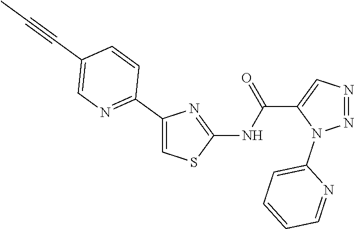 [SMILES: CC#Cc1ccc(-c2csc(NC(=O)c3cnnn3-c3ccccn3)n2)nc1]

Acid CAS 1480445-52-0
Amine CAS 2592824-00-3

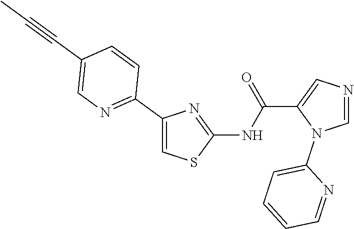 [SMILES: CC#Cc1ccc(-c2csc(NC(=O)c3cncn3-c3ccccn3)n2)nc1]

Acid CAS 1779128-15-2
Amine CAS 2592824-00-3

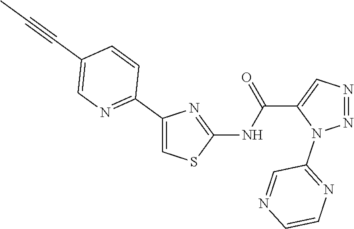 [SMILES: CC#Cc1ccc(-c2csc(NC(=O)c3cnnn3-c3cnccn3)n2)nc1]

Acid CAS 2946657-15-2
Amine CAS 2592824-00-3

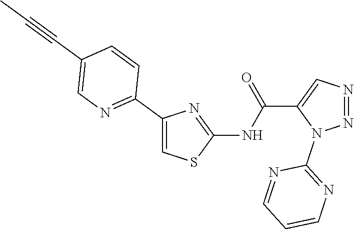 [SMILES: CC#Cc1ccc(-c2csc(NC(=O)c3cnnn3-c3ncccn3)n2)nc1]

Acid CAS 2946657-14-1
Amine CAS 2592824-00-3

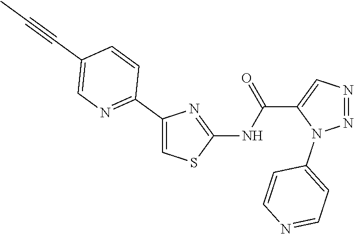 [SMILES: CC#Cc1ccc(-c2csc(NC(=O)c3cnnn3-c3ccncc3)n2)nc1]

Acid CAS 1525417-94-0
Amine CAS 2592824-00-3

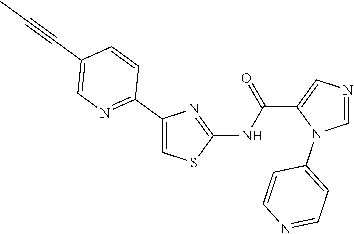 [SMILES: CC#Cc1ccc(-c2csc(NC(=O)c3cncn3-c3ccncc3)n2)nc1]

Acid CAS 1439900-49-8
Amine CAS 2592824-00-3

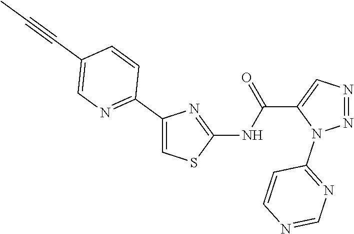 [SMILES: CC#Cc1ccc(-c2csc(NC(=O)c3cnnn3-c3ccncn3)n2)nc1]

Acid CAS 1482934-49-5
Amine CAS 2592824-00-3

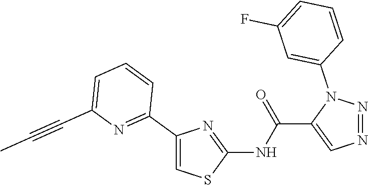 [SMILES: CC#Cc1cccc(-c2csc(NC(=O)c3cnnn3-c3cccc(F)c3)n2)n1]

Acid CAS 1153490-92-6
Amine CAS 2600157-36-4

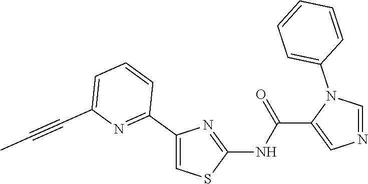 [SMILES: CC#Cc1cccc(-c2csc(NC(=O)c3cncn3-c3ccccc3)n2)n1]

Acid CAS 135417-65-1
Amine CAS 2600157-36-4

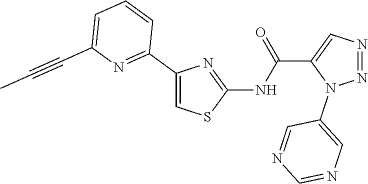 [SMILES: CC#Cc1cccc(-c2csc(NC(=O)c3cnnn3-c3cncnc3)n2)n1]

Acid CAS 147 [symbol] 093-94-5
Amine CAS 2600157-36-4

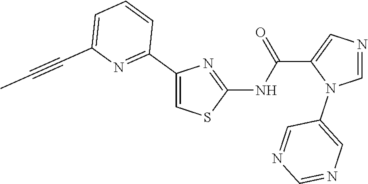 [SMILES: CC#Cc1cccc(-c2csc(NC(=O)c3cncn3-c3cncnc3)n2)n1]

Acid CAS 1523297-19-9
Amine CAS 2600157-36-4

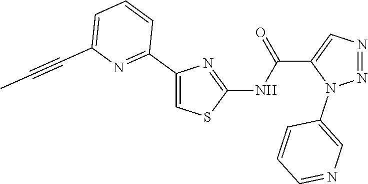 [SMILES: CC#Cc1cccc(-c2csc(NC(=O)c3cnnn3-c3cccnc3)n2)n1]

Acid CAS 147 [symbol] 271-10-1
Amine CAS 2600157-36-4

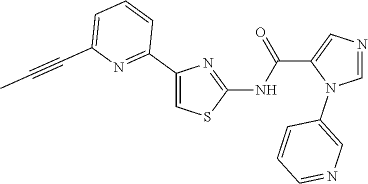 [SMILES: CC#Cc1cccc(-c2csc(NC(=O)c3cncn3-c3cccnc3)n2)n1]

Acid CAS 119739 [symbol] -67-6
Amine CAS 2600157-36-4

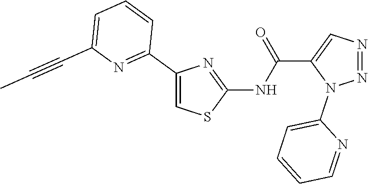 [SMILES: CC#Cc1cccc(-c2csc(NC(=O)c3cnnn3-c3ccccn3)n2)n1]

Acid CAS 14 [symbol] 445-52-0
Amine CAS 2600157-36-4

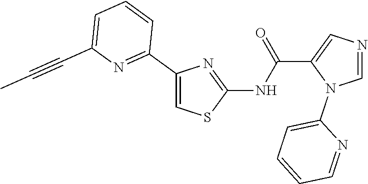 [SMILES: CC#Cc1cccc(-c2csc(NC(=O)c3cncn3-c3ccccn3)n2)n1]

Acid CAS 1779128-15-2
Amine CAS 2600157-36-4

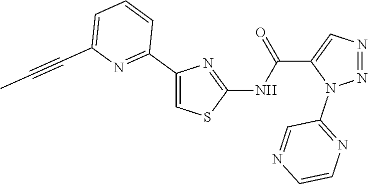 [SMILES: CC#Cc1cccc(-c2csc(NC(=O)c3cnnn3-c3cnccn3)n2)n1]

Acid CAS 2946657-15-2
Amine CAS 2600157-36-4

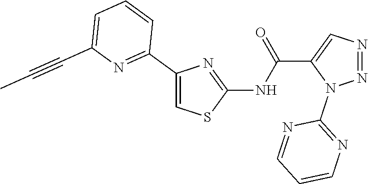 [SMILES: CC#Cc1cccc(-c2csc(NC(=O)c3cnnn3-c3ncccn3)n2)n1]

Acid CAS 2946657-14-1
Amine CAS 2600157-36-4

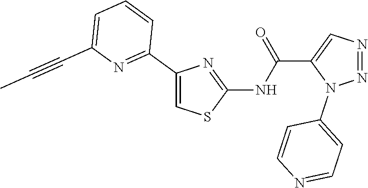 [SMILES: CC#Cc1cccc(-c2csc(NC(=O)c3cnnn3-c3ccncc3)n2)n1]

Acid CAS 1525417-94-0
Amine CAS 2600157-36-4

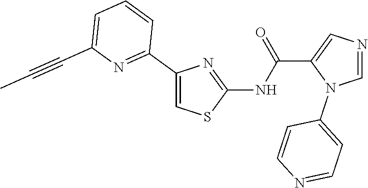 [SMILES: CC#Cc1cccc(-c2csc(NC(=O)c3cncn3-c3ccncc3)n2)n1]

Acid CAS 14 [symbol] 9900-49-8
Amine CAS 2600157-36-4

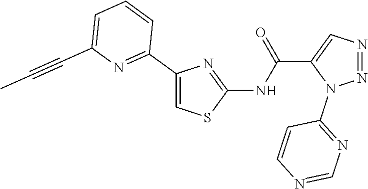 [SMILES: CC#Cc1cccc(-c2csc(NC(=O)c3cnnn3-c3ccncn3)n2)n1]

Acid CAS 1482934-49-5
Amine CAS 2600157-36-4

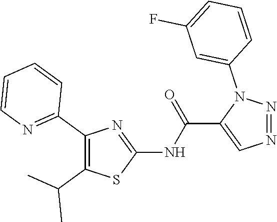 [SMILES: CC(C)c1sc(NC(=O)c2cnnn2-c2cccc(F)c2)nc1-c1ccccn1]

Acid CAS 1153490-92-6
Amine CAS 1225774-92-4

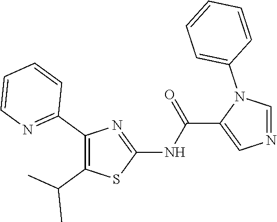 [SMILES: CC(C)c1sc(NC(=O)c2cncn2-c2ccccc2)nc1-c1ccccn1]

Acid CAS 135417-65-1
Amine CAS 1225774-92-4

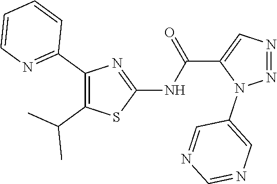 [SMILES: CC(C)c1sc(NC(=O)c2cnnn2-c2cncnc2)nc1-c1ccccn1]

Acid CAS 147 [symbol] 093-94-5
Amine CAS 1225774-92-4

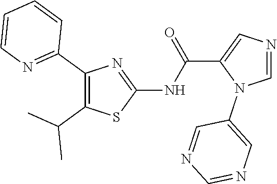 [SMILES: CC(C)c1sc(NC(=O)c2cncn2-c2cncnc2)nc1-c1ccccn1]

Acid CAS 1523297-19-9
Amine CAS 1225774-92-4

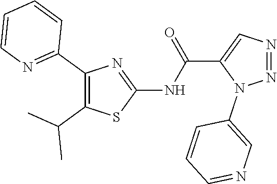 [SMILES: CC(C)c1sc(NC(=O)c2cnnn2-c2cccnc2)nc1-c1ccccn1]

Acid CAS 147 [symbol] 271-10-1
Amine CAS 1225774-92-4

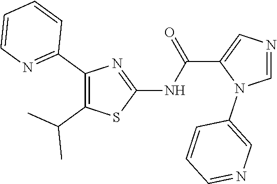 [SMILES: CC(C)c1sc(NC(=O)c2cncn2-c2cccnc2)nc1-c1ccccn1]

Acid CAS 1197398-67-6
Amine CAS 1225774-92-4

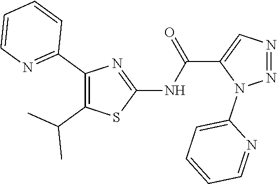 [SMILES: CC(C)c1sc(NC(=O)c2cnnn2-c2ccccn2)nc1-c1ccccn1]

Acid CAS 1480445-52-0
Amine CAS 1225774-92-4

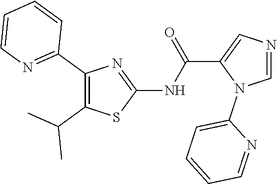 [SMILES: CC(C)c1sc(NC(=O)c2cncn2-c2ccccn2)nc1-c1ccccn1]

Acid CAS 1779128-15-2
Amine CAS 1225774-92-4

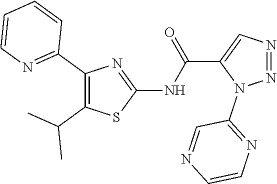 [SMILES: CC(C)c1sc(NC(=O)c2cnnn2-c2cnccn2)nc1-c1ccccn1]

Acid CAS 2946657-15-2
Amine CAS 1225774-92-4

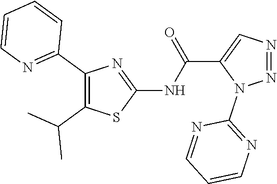 [SMILES: CC(C)c1sc(NC(=O)c2cnnn2-c2ncccn2)nc1-c1ccccn1]

Acid CAS 2946657-14-1
Amine CAS 1225774-92-4

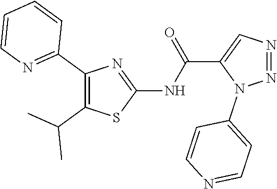 [SMILES: CC(C)c1sc(NC(=O)c2cnnn2-c2ccncc2)nc1-c1ccccn1]

Acid CAS 1525417-94-0
Amine CAS 1225774-92-4

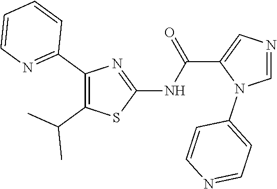 [SMILES: CC(C)c1sc(NC(=O)c2cncn2-c2ccncc2)nc1-c1ccccn1]

Acid CAS 1439900-49-8
Amine CAS 1225774-92-4

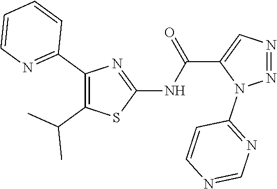 [SMILES: CC(C)c1sc(NC(=O)c2cnnn2-c2ccncn2)nc1-c1ccccn1]

Acid CAS 1482934-49-5
Amine CAS 1225774-92-4

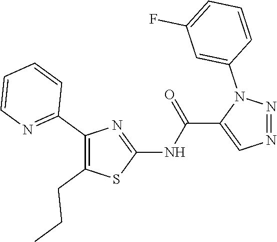 [SMILES: CCCc1sc(NC(=O)c2cnnn2-c2cccc(F)c2)nc1-c1ccccn1]

Acid CAS 1153490-92-6
Amine CAS 2042701-33-5

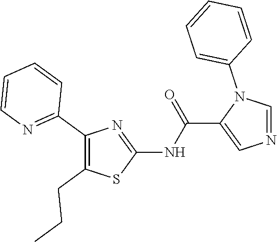 [SMILES: CCCc1sc(NC(=O)c2cncn2-c2ccccc2)nc1-c1ccccn1]

Acid CAS 135417-65-1
Amine CAS 2042701-33-5

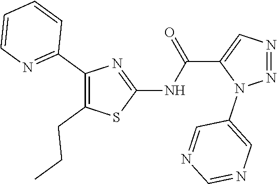 [SMILES: CCCc1sc(NC(=O)c2cnnn2-c2cncnc2)nc1-c1ccccn1]

Acid CAS 147 [symbol] 093-94-5
Amine CAS 2042701-33-5

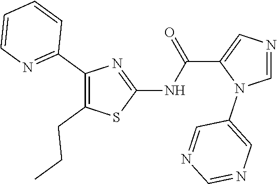 [SMILES: CCCc1sc(NC(=O)c2cncn2-c2cncnc2)nc1-c1ccccn1]

Acid CAS 1523297-19-9
Amine CAS 2042701-33-5

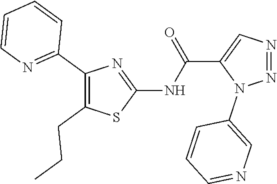 [SMILES: CCCc1sc(NC(=O)c2cnnn2-c2cccnc2)nc1-c1ccccn1]

Acid CAS 1476271-10-1
Amine CAS 2042701-33-5

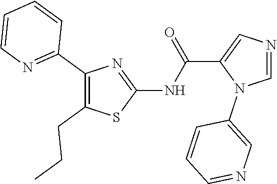 [SMILES: CCCc1sc(NC(=O)c2cncn2-c2cccnc2)nc1-c1ccccn1]

Acid CAS 1197398-67-6
Amine CAS 2042701-33-5

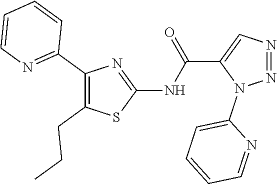 [SMILES: CCCc1sc(NC(=O)c2cnnn2-c2ccccn2)nc1-c1ccccn1]

Acid CAS 1480445-52-0
Amine CAS 2042701-33-5

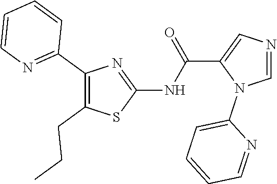 [SMILES: CCCc1sc(NC(=O)c2cncn2-c2ccccn2)nc1-c1ccccn1]

Acid CAS 1779128-15-2
Amine CAS 2042701-33-5

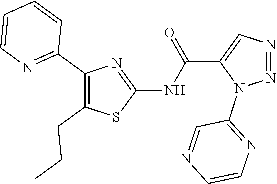 [SMILES: CCCc1sc(NC(=O)c2cnnn2-c2cnccn2)nc1-c1ccccn1]

Acid CAS 2946657-15-2
Amine CAS 2042701-33-5

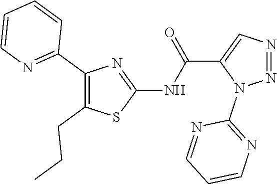 [SMILES: CCCc1sc(NC(=O)c2cnnn2-c2ncccn2)nc1-c1ccccn1]

Acid CAS 2946657-14-1
Amine CAS 2042701-33-5

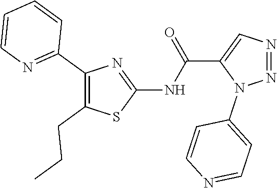 [SMILES: CCCc1sc(NC(=O)c2cnnn2-c2ccncc2)nc1-c1ccccn1]

Acid CAS 1525417-94-0
Amine CAS 2042701-33-5

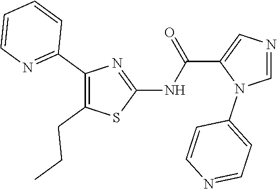 [SMILES: CCCc1sc(NC(=O)c2cncn2-c2ccncc2)nc1-c1ccccn1]

Acid CAS 1439900-49-8
Amine CAS 2042701-33-5

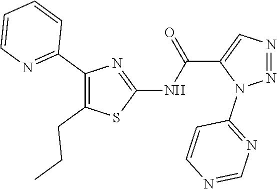 [SMILES: CCCc1sc(NC(=O)c2cnnn2-c2ccncn2)nc1-c1ccccn1]

Acid CAS 1482934-49-5
Amine CAS 2042701-33-5

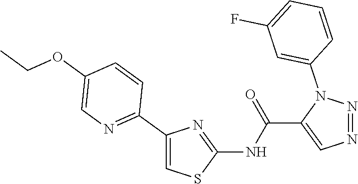 [SMILES: CCOc1ccc(-c2csc(NC(=O)c3cnnn3-c3cccc(F)c3)n2)nc1]

Acid CAS 1153490-92-6
Amine CAS 2768587-25-1

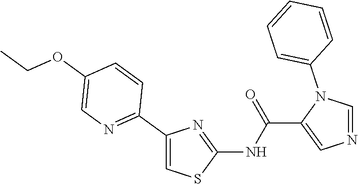 [SMILES: CCOc1ccc(-c2csc(NC(=O)c3cncn3-c3ccccc3)n2)nc1]

Acid CAS 135417-65-1
Amine CAS 2768587-25-1

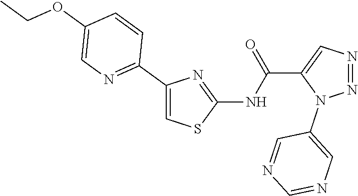 [SMILES: CCOc1ccc(-c2csc(NC(=O)c3cnnn3-c3cncnc3)n2)nc1]

Acid CAS 1478096-94-5
Amine CAS 2768587-25-1

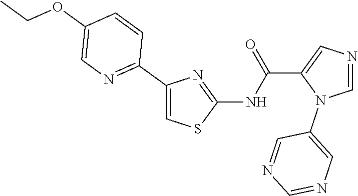 [SMILES: CCOc1ccc(-c2csc(NC(=O)c3cncn3-c3cncnc3)n2)nc1]

Acid CAS 1523297-19-9
Amine CAS 2768587-25-1

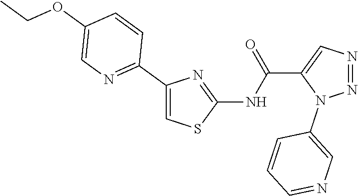 [SMILES: CCOc1ccc(-c2csc(NC(=O)c3cnnn3-c3cccnc3)n2)nc1]

Acid CAS 1478271-10-1
Amine CAS 2768587-25-1

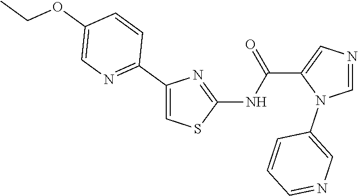 [SMILES: CCOc1ccc(-c2csc(NC(=O)c3cncn3-c3cccnc3)n2)nc1]

Acid CAS 1197398-67-6
Amine CAS 2768587-25-1

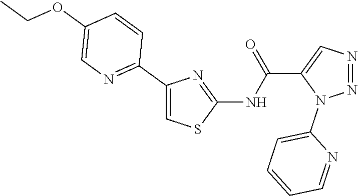 [SMILES: CCOc1ccc(-c2csc(NC(=O)c3cnnn3-c3ccccn3)n2)nc1]

Acid CAS 1480445-52-0
Amine CAS 2768587-25-1

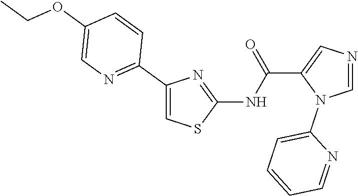 [SMILES: CCOc1ccc(-c2csc(NC(=O)c3cncn3-c3ccccn3)n2)nc1]

Acid CAS 1779128-15-2
Amine CAS 2768587-25-1

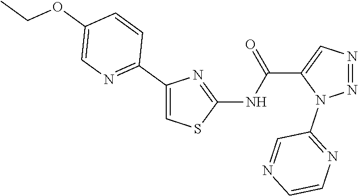 [SMILES: CCOc1ccc(-c2csc(NC(=O)c3cnnn3-c3cnccn3)n2)nc1]

Acid CAS 2946657-15-2
Amine CAS 2768587-25-1

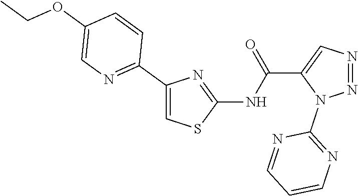 [SMILES: CCOc1ccc(-c2csc(NC(=O)c3cnnn3-c3ncccn3)n2)nc1]

Acid CAS 2946657-14-1
Amine CAS 2768587-25-1

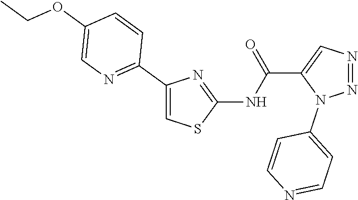 [SMILES: CCOc1ccc(-c2csc(NC(=O)c3cnnn3-c3ccncc3)n2)nc1]

Acid CAS 1525417-94-0
Amine CAS 2768587-25-1

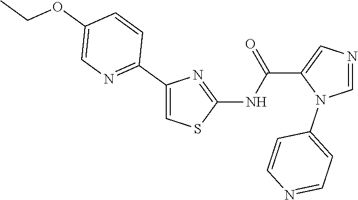 [SMILES: CCOc1ccc(-c2csc(NC(=O)c3cncn3-c3ccncc3)n2)nc1]

Acid CAS 1439900-49-8
Amine CAS 2768587-25-1

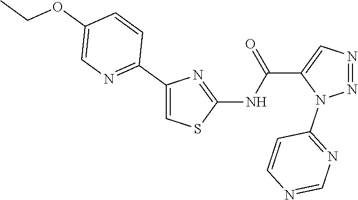 [SMILES: CCOc1ccc(-c2csc(NC(=O)c3cnnn3-c3ccncn3)n2)nc1]

Acid CAS 1482934-49-5
Amine CAS 2768587-25-1

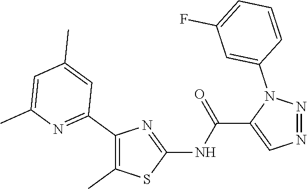 [SMILES: Cc1cc(C)nc(-c2nc(NC(=O)c3cnnn3-c3cccc(F)c3)sc2C)c1]

Acid CAS 1153490-92-6
Amine CAS 2267501-11-3

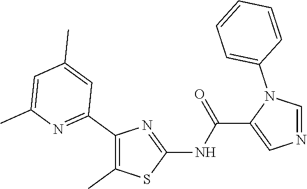 [SMILES: Cc1cc(C)nc(-c2nc(NC(=O)c3cncn3-c3ccccc3)sc2C)c1]

Acid CAS 135417-65-1
Amine CAS 2287501-11-3

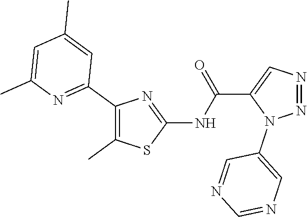 [SMILES: Cc1cc(C)nc(-c2nc(NC(=O)c3cnnn3-c3cncnc3)sc2C)c1]

Acid CAS 1478093-94-5
Amine CAS 2287501-11-3

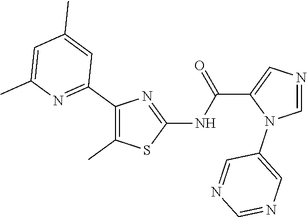 [SMILES: Cc1cc(C)nc(-c2nc(NC(=O)c3cncn3-c3cncnc3)sc2C)c1]

Acid CAS 1523297-19-9
Amine CAS 2287501-11-3

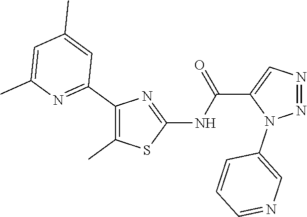 [SMILES: Cc1cc(C)nc(-c2nc(NC(=O)c3cnnn3-c3cccnc3)sc2C)c1]

Acid CAS 1478271-10-1
Amine CAS 2287501-11-3

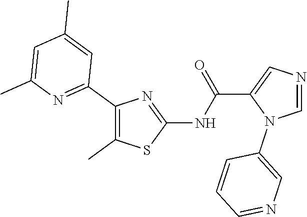 [SMILES: Cc1cc(C)nc(-c2nc(NC(=O)c3cncn3-c3cccnc3)sc2C)c1]

Acid CAS 1197398-67-6
Amine CAS 2287501-11-3

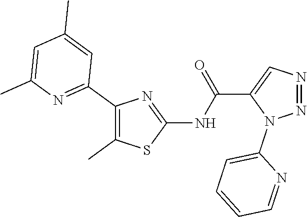 [SMILES: Cc1cc(C)nc(-c2nc(NC(=O)c3cnnn3-c3ccccn3)sc2C)c1]

Acid CAS 1480445-52-0
Amine CAS 2287501-11-3

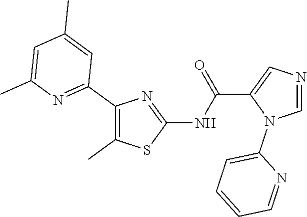 [SMILES: Cc1cc(C)nc(-c2nc(NC(=O)c3cncn3-c3ccccn3)sc2C)c1]

Acid CAS 1779128-15-2
Amine CAS 2287501-11-3

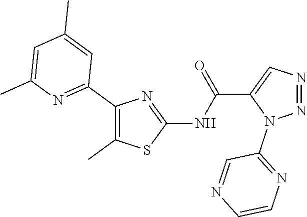 [SMILES: Cc1cc(C)nc(-c2nc(NC(=O)c3cnnn3-c3cnccn3)sc2C)c1]

Acid CAS 2946657-15-2
Amine CAS 2287501-11-3

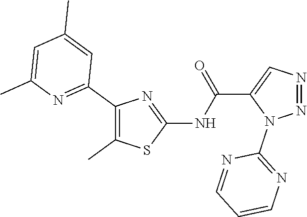 [SMILES: Cc1cc(C)nc(-c2nc(NC(=O)c3cnnn3-c3ncccn3)sc2C)c1]

Acid CAS 2946657-14-1
Amine CAS 2287501-11-3

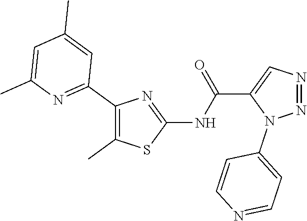 [SMILES: Cc1cc(C)nc(-c2nc(NC(=O)c3cnnn3-c3ccncc3)sc2C)c1]

Acid CAS 1525417-94-0
Amine CAS 2287501-11-3

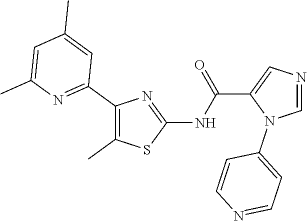 [SMILES: Cc1cc(C)nc(-c2nc(NC(=O)c3cncn3-c3ccncc3)sc2C)c1]

Acid CAS 1439900-49-8
Amine CAS 2287501-11-3

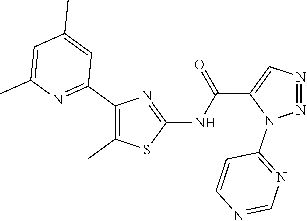 [SMILES: Cc1cc(C)nc(-c2nc(NC(=O)c3cnnn3-c3ccncn3)sc2C)c1]

Acid CAS 1482934-49-5
Amine CAS 2287501-11-3

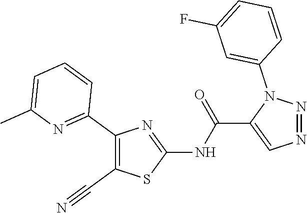 [SMILES: Cc1cccc(-c2nc(NC(=O)c3cnnn3-c3cccc(F)c3)sc2C#N)n1]

Acid CAS 1153490-92-6
Amine CAS 1975266-69-3

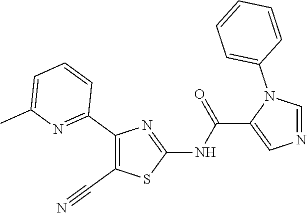 [SMILES: Cc1cccc(-c2nc(NC(=O)c3cncn3-c3ccccc3)sc2C#N)n1]

Acid CAS 135417-65-1
Amine CAS 1975266-69-3

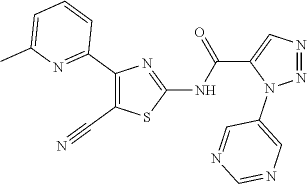 [SMILES: Cc1cccc(-c2nc(NC(=O)c3cnnn3-c3cncnc3)sc2C#N)n1]

Acid CAS 1478093-94-5
Amine CAS 1975266-69-3

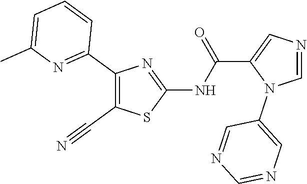 [SMILES: Cc1cccc(-c2nc(NC(=O)c3cncn3-c3cncnc3)sc2C#N)n1]

Acid CAS 1523297-19-9
Amine CAS 1975266-69-3

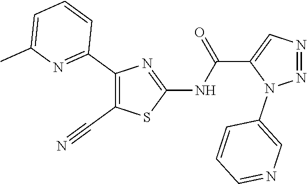 [SMILES: Cc1cccc(-c2nc(NC(=O)c3cnnn3-c3cccnc3)sc2C#N)n1]

Acid CAS 1478271-10-1
Amine CAS 1975266-69-3

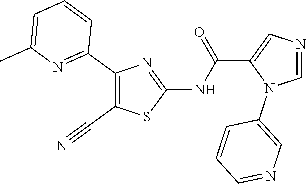 [SMILES: Cc1cccc(-c2nc(NC(=O)c3cncn3-c3cccnc3)sc2C#N)n1]

Acid CAS 119739 [symbol] -67-6
Amine CAS 1975266-69-3

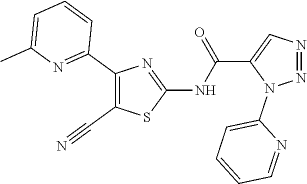 [SMILES: Cc1cccc(-c2nc(NC(=O)c3cnnn3-c3ccccn3)sc2C#N)n1]

Acid CAS 14 [symbol] 0445-52-0
Amine CAS 1975266-69-3

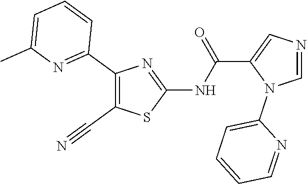 [SMILES: Cc1cccc(-c2nc(NC(=O)c3cncn3-c3ccccn3)sc2C#N)n1]

Acid CAS 1779128-15-2
Amine CAS 1975266-69-3

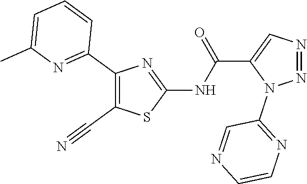 [SMILES: Cc1cccc(-c2nc(NC(=O)c3cnnn3-c3cnccn3)sc2C#N)n1]

Acid CAS 2946657-15-2
Amine CAS 1975266-69-3

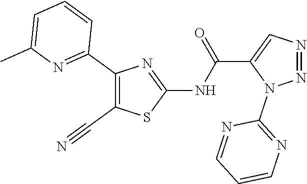 [SMILES: Cc1cccc(-c2nc(NC(=O)c3cnnn3-c3ncccn3)sc2C#N)n1]

Acid CAS 2946657-14-1
Amine CAS 1975266-69-3

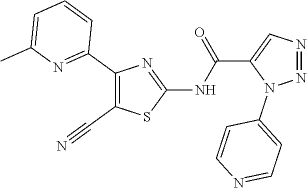 [SMILES: Cc1cccc(-c2nc(NC(=O)c3cnnn3-c3ccncc3)sc2C#N)n1]

Acid CAS 1525417-94-0
Amine CAS 1975268-69-3

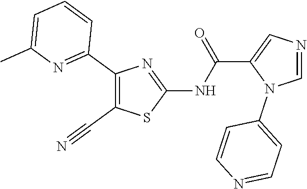 [SMILES: Cc1cccc(-c2nc(NC(=O)c3cncn3-c3ccncc3)sc2C#N)n1]

Acid CAS 1439900-49-8
Amine CAS 1975266-69-3

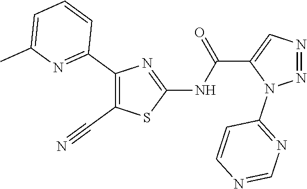 [SMILES: Cc1cccc(-c2nc(NC(=O)c3cnnn3-c3ccncn3)sc2C#N)n1]

Acid CAS 1482934-49-5
Amine CAS 1975266-69-3

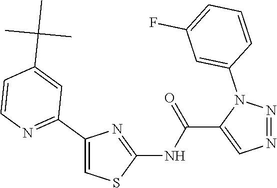 [SMILES: CC(C)(C)c1ccnc(-c2csc(NC(=O)c3cnnn3-c3cccc(F)c3)n2)c1]

Acid CAS 1153490-92-6
Amine CAS 2253868-94-7

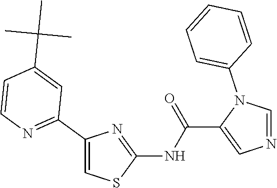 [SMILES: CC(C)(C)c1ccnc(-c2csc(NC(=O)c3cncn3-c3ccccc3)n2)c1]

Acid CAS 135417-65-1
Amine CAS 2253868-94-7

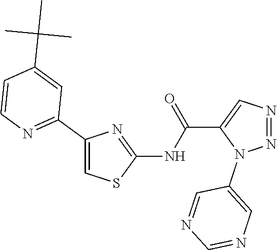 [SMILES: CC(C)(C)c1ccnc(-c2csc(NC(=O)c3cnnn3-c3cncnc3)n2)c1]

Acid CAS 1478093-94-5
Amine CAS 2253868-94-7

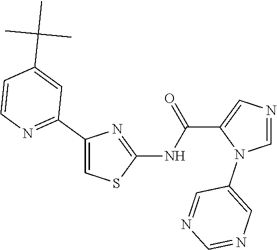 [SMILES: CC(C)(C)c1ccnc(-c2csc(NC(=O)c3cncn3-c3cncnc3)n2)c1]

Acid CAS 1523297-19-9
Amine CAS 2253868-94-7

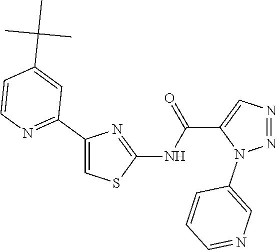 [SMILES: CC(C)(C)c1ccnc(-c2csc(NC(=O)c3cnnn3-c3cccnc3)n2)c1]

Acid CAS 147 [symbol] 271-10-1
Amine CAS 2253868-94-7

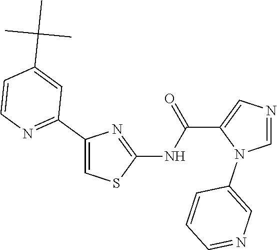 [SMILES: CC(C)(C)c1ccnc(-c2csc(NC(=O)c3cncn3-c3cccnc3)n2)c1]

Acid CAS 1197398-67-6
Amine CAS 2253868-94-7

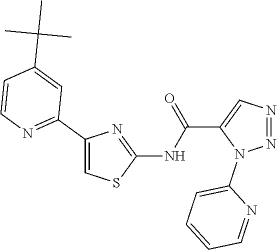 [SMILES: CC(C)(C)c1ccnc(-c2csc(NC(=O)c3cnnn3-c3ccccn3)n2)c1]

Acid CAS 14 [symbol] 0445-52-0
Amine CAS 2253868-94-7

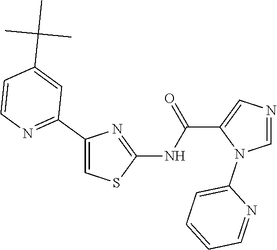 [SMILES: CC(C)(C)c1ccnc(-c2csc(NC(=O)c3cncn3-c3ccccn3)n2)c1]

Acid CAS 1779128-15-2
Amine CAS 2253868-94-7

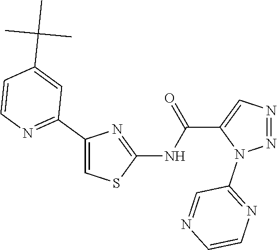 [SMILES: CC(C)(C)c1ccnc(-c2csc(NC(=O)c3cnnn3-c3cnccn3)n2)c1]

Acid CAS 2946657-15-2
Amine CAS 2253868-94-7

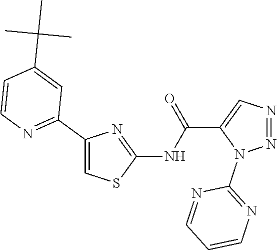 [SMILES: CC(C)(C)c1ccnc(-c2csc(NC(=O)c3cnnn3-c3ncccn3)n2)c1]

Acid CAS 2946657-14-1
Amine CAS 2253868-94-7

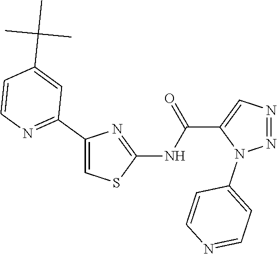 [SMILES: CC(C)(C)c1ccnc(-c2csc(NC(=O)c3cnnn3-c3ccncc3)n2)c1]

Acid CAS 1525417-94-0
Amine CAS 2253868-94-7

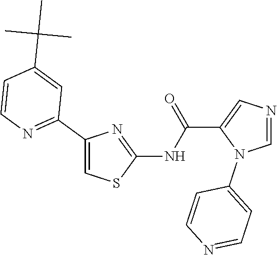 [SMILES: CC(C)(C)c1ccnc(-c2csc(NC(=O)c3cncn3-c3ccncc3)n2)c1]

Acid CAS 1439900-49-8
Amine CAS 2253868-94-7

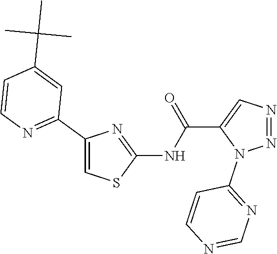 [SMILES: CC(C)(C)c1ccnc(-c2csc(NC(=O)c3cnnn3-c3ccncn3)n2)c1]

Acid CAS 1462934-49-5
Amine CAS 2253868-94-7

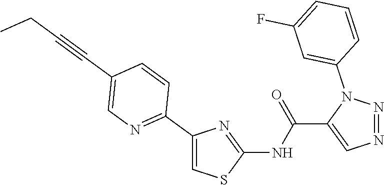 [SMILES: CCC#Cc1ccc(-c2csc(NC(=O)c3cnnn3-c3cccc(F)c3)n2)nc1]

Acid CAS 1153490-92- [symbol]
Amine CAS 2538808-37-4

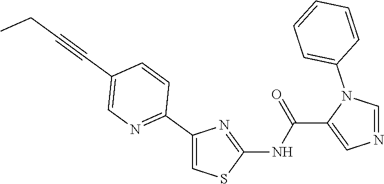 [SMILES: CCC#Cc1ccc(-c2csc(NC(=O)c3cncn3-c3ccccc3)n2)nc1]

Acid CAS 135417-65-1
Amine CAS 2538808-37-4

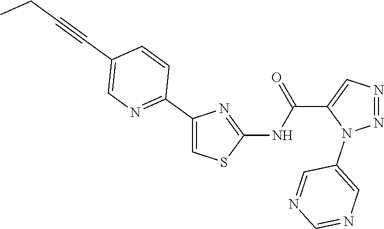 [SMILES: CCC#Cc1ccc(-c2csc(NC(=O)c3cnnn3-c3cncnc3)n2)nc1]

Acid CAS 1478093-94-5
Amine CAS 2538808-37-4

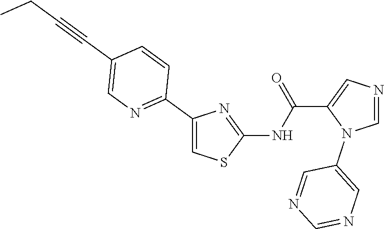 [SMILES: CCC#Cc1ccc(-c2csc(NC(=O)c3cncn3-c3cncnc3)n2)nc1]

Acid CAS 1523297-19-9
Amine CAS 2538808-37-4

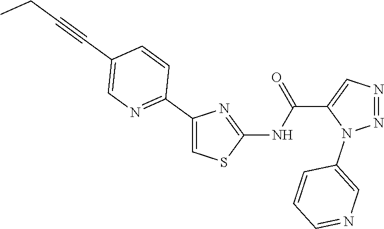 [SMILES: CCC#Cc1ccc(-c2csc(NC(=O)c3cnnn3-c3cccnc3)n2)nc1]

Acid CAS 1478271-10-1
Amine CAS 2538808-37-4

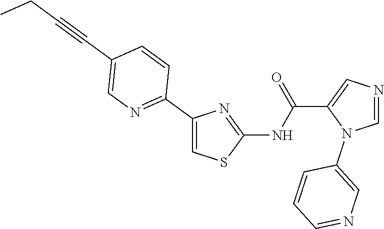 [SMILES: CCC#Cc1ccc(-c2csc(NC(=O)c3cncn3-c3cccnc3)n2)nc1]

Acid CAS 1197398-67-6
Amine CAS 2538808-37-4

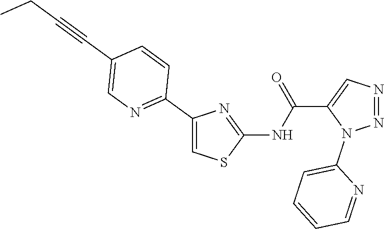 [SMILES: CCC#Cc1ccc(-c2csc(NC(=O)c3cnnn3-c3ccccn3)n2)nc1]

Acid CAS 1480445-52-0
Amine CAS 2538808-37-4

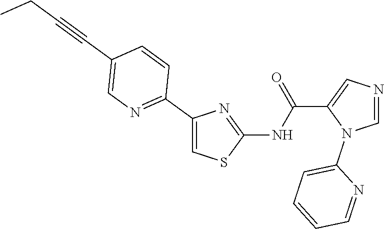 [SMILES: CCC#Cc1ccc(-c2csc(NC(=O)c3cncn3-c3ccccn3)n2)nc1]

Acid CAS 1179128-15-2
Amine CAS 2538808-37-4

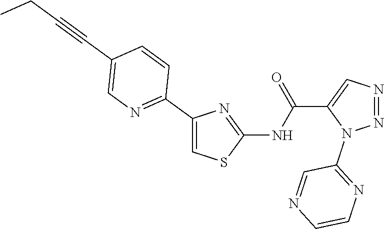 [SMILES: CCC#Cc1ccc(-c2csc(NC(=O)c3cnnn3-c3cnccn3)n2)nc1]

Acid CAS 2946657-15-2
Amine CAS 2538808-37-4

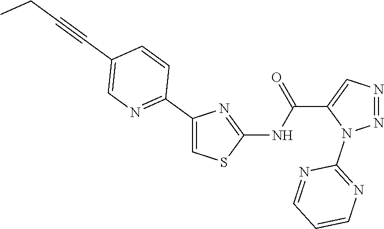 [SMILES: CCC#Cc1ccc(-c2csc(NC(=O)c3cnnn3-c3ncccn3)n2)nc1]

Acid CAS 2946657-14-1
Amine CAS 2538808-37-4

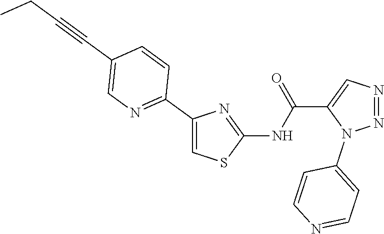 [SMILES: CCC#Cc1ccc(-c2csc(NC(=O)c3cnnn3-c3ccncc3)n2)nc1]

Acid CAS 1525417-94-0
Amine CAS 2538808-37-4

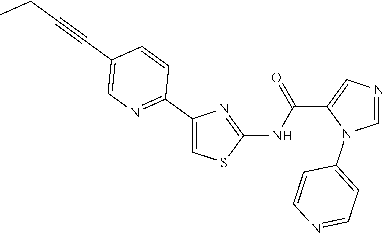 [SMILES: CCC#Cc1ccc(-c2csc(NC(=O)c3cncn3-c3ccncc3)n2)nc1]

Acid CAS 1439900-48-8
Amine CAS 2538808-37-4

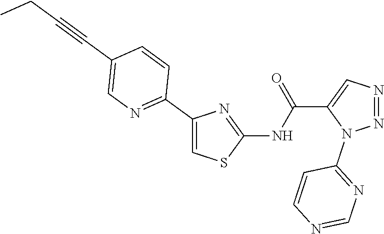 [SMILES: CCC#Cc1ccc(-c2csc(NC(=O)c3cnnn3-c3ccncn3)n2)nc1]

Acid CAS 1482934-49-5
Amine CAS 2538808-37-4

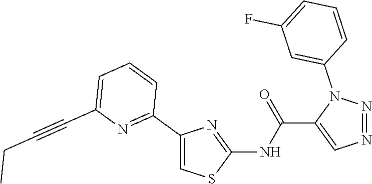 [SMILES: CCC#Cc1cccc(-c2csc(NC(=O)c3cnnn3-c3cccc(F)c3)n2)n1]

Acid CAS 1153490-92-6
Amine CAS 2539204-54-9

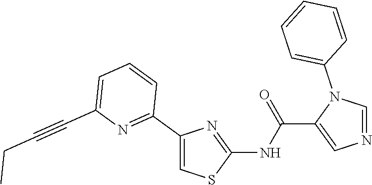 [SMILES: CCC#Cc1cccc(-c2csc(NC(=O)c3cncn3-c3ccccc3)n2)n1]

Acid CAS 135417-65-1
Amine CAS 2539204-54-9

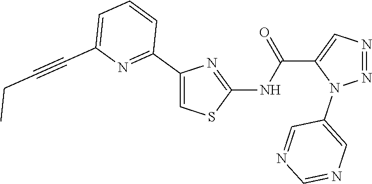 [SMILES: CCC#Cc1cccc(-c2csc(NC(=O)c3cnnn3-c3cncnc3)n2)n1]

Acid CAS 1478093-94-5
Amine CAS 2539204-54-9

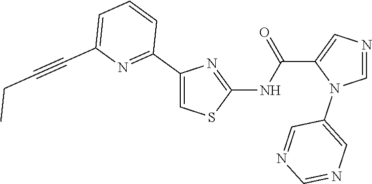 [SMILES: CCC#Cc1cccc(-c2csc(NC(=O)c3cncn3-c3cncnc3)n2)n1]

Acid CAS 1523297-19-9
Amine CAS 2539204-54-9

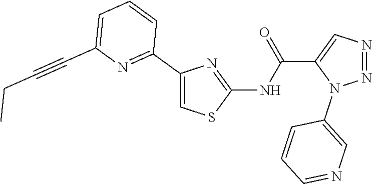 [SMILES: CCC#Cc1cccc(-c2csc(NC(=O)c3cnnn3-c3cccnc3)n2)n1]

Acid CAS 1478271-10-1
Amine CAS 2539204-54-9

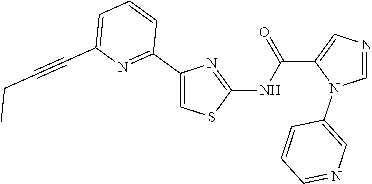 [SMILES: CCC#Cc1cccc(-c2csc(NC(=O)c3cncn3-c3cccnc3)n2)n1]

Acid CAS 1197398-67-6
Amine CAS 2539204-54-9

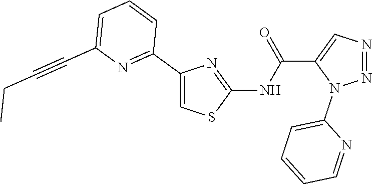 [SMILES: CCC#Cc1cccc(-c2csc(NC(=O)c3cnnn3-c3ccccn3)n2)n1]

Acid CAS 14 [symbol] 0445-52-0
Amine CAS 2539204-54-9

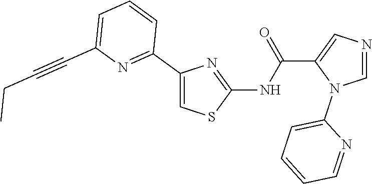 [SMILES: CCC#Cc1cccc(-c2csc(NC(=O)c3cncn3-c3ccccn3)n2)n1]

Acid CAS 1779128-15-2
Amine CAS 2539204-54-9

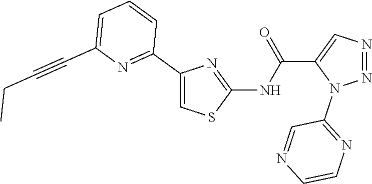 [SMILES: CCC#Cc1cccc(-c2csc(NC(=O)c3cnnn3-c3cnccn3)n2)n1]

Acid CAS 2946657-15-2
Amine CAS 2539204-54-9

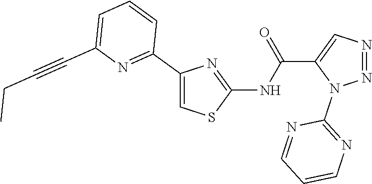 [SMILES: CCC#Cc1cccc(-c2csc(NC(=O)c3cnnn3-c3ncccn3)n2)n1]

Acid CAS 2946657-14-1
Amine CAS 2539204-54-9

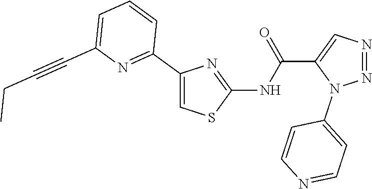 [SMILES: CCC#Cc1cccc(-c2csc(NC(=O)c3cnnn3-c3ccncc3)n2)n1]

Acid CAS 1525417-94-0
Amine CAS 2539204-54-9

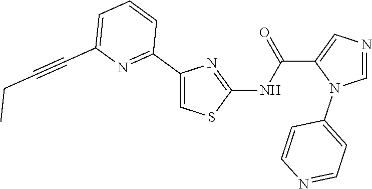 [SMILES: CCC#Cc1cccc(-c2csc(NC(=O)c3cncn3-c3ccncc3)n2)n1]

Acid CAS 1439900-49-8
Amine CAS 2539204-54-9

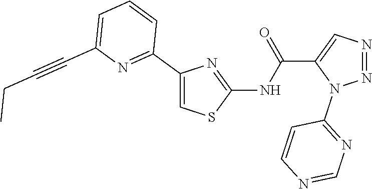 [SMILES: CCC#Cc1cccc(-c2csc(NC(=O)c3cnnn3-c3ccncn3)n2)n1]

Acid CAS 1482934-49-5
Amine CAS 2539204-54-9

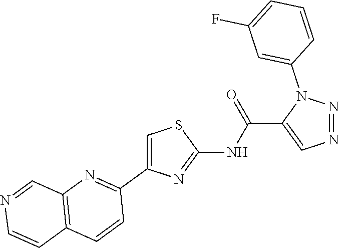 [SMILES: O=C(Nc1nc(-c2ccc3ccncc3n2)cs1)c1cnnn1-c1cccc(F)c1]

Acid CAS 1153490-92-6
Amine CAS 2604301-23-5

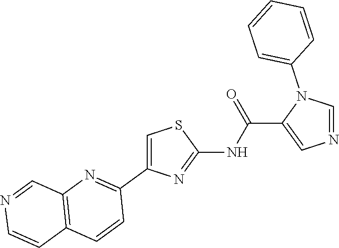 [SMILES: O=C(Nc1nc(-c2ccc3ccncc3n2)cs1)c1cncn1-c1ccccc1]

Acid CAS 135417-65-1
Amine CAS 2604301-23-5

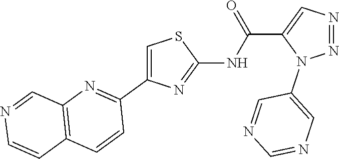 [SMILES: O=C(Nc1nc(-c2ccc3ccncc3n2)cs1)c1cnnn1-c1cncnc1]

Acid CAS 1478093-94-5
Amine CAS 2604301-23-5

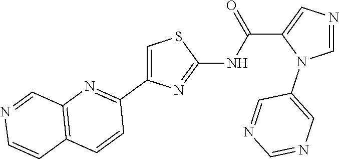 [SMILES: O=C(Nc1nc(-c2ccc3ccncc3n2)cs1)c1cncn1-c1cncnc1]

Acid CAS 1523297-19-9
Amine CAS 2604301-23-5

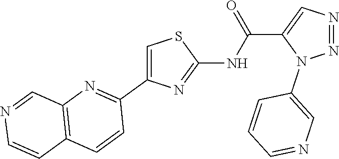 [SMILES: O=C(Nc1nc(-c2ccc3ccncc3n2)cs1)c1cnnn1-c1cccnc1]

Acid CAS 1478271-10-1
Amine CAS 2604301-23-5

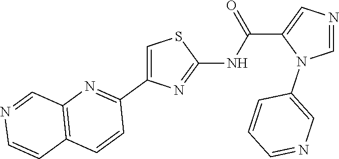 [SMILES: O=C(Nc1nc(-c2ccc3ccncc3n2)cs1)c1cncn1-c1cccnc1]

Acid CAS 1197398-67-6
Amine CAS 2604301-23-5

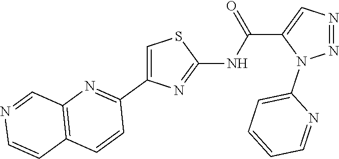 [SMILES: O=C(Nc1nc(-c2ccc3ccncc3n2)cs1)c1cnnn1-c1ccccn1]

Acid CAS 1480445-52-0
Amine CAS 2604301-23-5

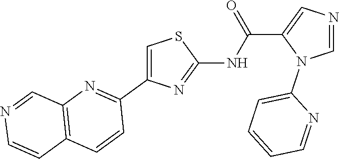 [SMILES: O=C(Nc1nc(-c2ccc3ccncc3n2)cs1)c1cncn1-c1ccccn1]

Acid CAS 1779128-15-2
Amine CAS 2604301-23-5

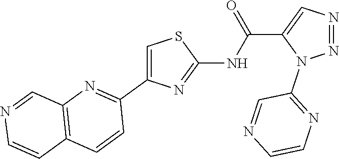 [SMILES: O=C(Nc1nc(-c2ccc3ccncc3n2)cs1)c1cnnn1-c1cnccn1]

Acid CAS 2946657-15-2
Amine CAS 2604301-23-5

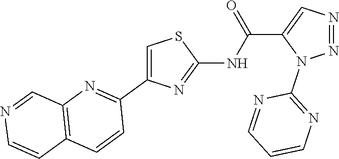 [SMILES: O=C(Nc1nc(-c2ccc3ccncc3n2)cs1)c1cnnn1-c1ncccn1]

Acid CAS 2946657-14-1
Amine CAS 2604301-23-5

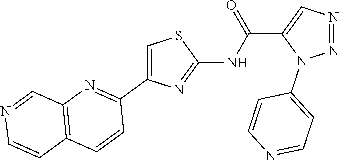 [SMILES: O=C(Nc1nc(-c2ccc3ccncc3n2)cs1)c1cnnn1-c1ccncc1]

Acid CAS 1525417-94-0
Amine CAS 2604301-23-5

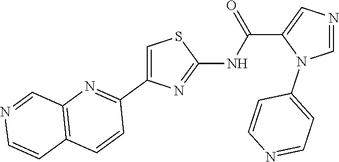 [SMILES: O=C(Nc1nc(-c2ccc3ccncc3n2)cs1)c1cncn1-c1ccncc1]

Acid CAS 1439900-49- [symbol]
Amine CAS 2604301-23-5

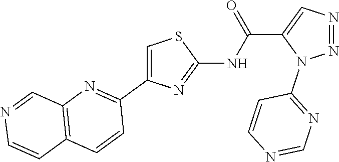 [SMILES: O=C(Nc1nc(-c2ccc3ccncc3n2)cs1)c1cnnn1-c1ccncn1]

Acid CAS 1482934-49-5
Amine CAS 2604301-23-5

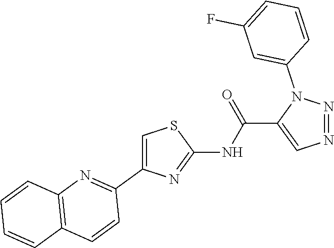 [SMILES: O=C(Nc1nc(-c2ccc3ccccc3n2)cs1)c1cnnn1-c1cccc(F)c1]

Acid CAS 1153490-92-6
Amine CAS 30235-29-1

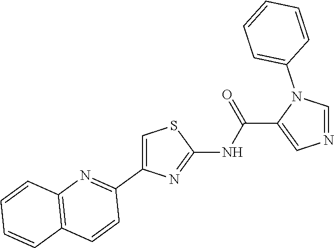 [SMILES: O=C(Nc1nc(-c2ccc3ccccc3n2)cs1)c1cncn1-c1ccccc1]

Acid CAS 135417-65-1
Amine CAS 30235-29-1

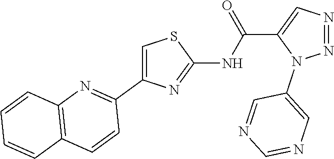 [SMILES: O=C(Nc1nc(-c2ccc3ccccc3n2)cs1)c1cnnn1-c1cncnc1]

Acid CAS 1478093-94-5
Amine CAS 30235-29-1

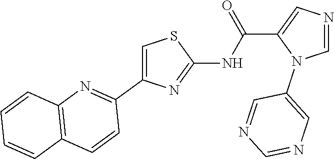 [SMILES: O=C(Nc1nc(-c2ccc3ccccc3n2)cs1)c1cncn1-c1cncnc1]

Acid CAS 1523297-19-9
Amine CAS 30235-29-1

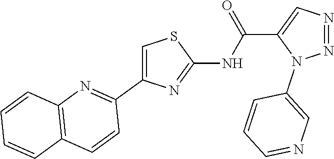 [SMILES: O=C(Nc1nc(-c2ccc3ccccc3n2)cs1)c1cnnn1-c1cccnc1]

Acid CAS 1478271-10-1
Amine CAS 30235-29-1

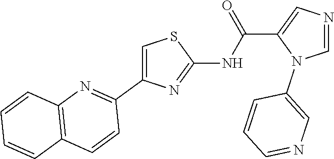 [SMILES: O=C(Nc1nc(-c2ccc3ccccc3n2)cs1)c1cncn1-c1cccnc1]

Acid CAS 1197398-67-6
Amine CAS 30235-29-1

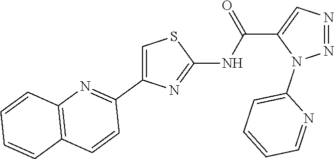 [SMILES: O=C(Nc1nc(-c2ccc3ccccc3n2)cs1)c1cnnn1-c1ccccn1]

Acid CAS 1480445-52-0
Amine CAS 30235-29-1

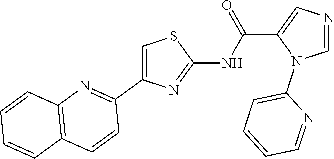 [SMILES: O=C(Nc1nc(-c2ccc3ccccc3n2)cs1)c1cncn1-c1ccccn1]

Acid CAS 1779128-15-2
Amine CAS 30235-29-1

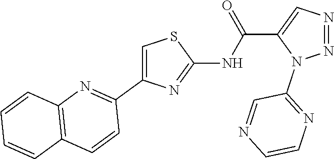 [SMILES: O=C(Nc1nc(-c2ccc3ccccc3n2)cs1)c1cnnn1-c1cnccn1]

Acid CAS 2946657-15-2
Amine CAS 30235-29-1

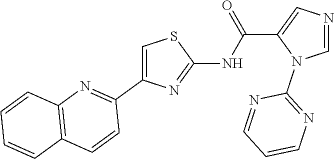 [SMILES: O=C(Nc1nc(-c2ccc3ccccc3n2)cs1)c1cncn1-c1ncccn1]

Acid CAS 2946657-14-1
Amine CAS 30235-29-1

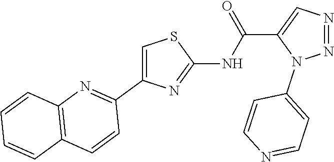 [SMILES: O=C(Nc1nc(-c2ccc3ccccc3n2)cs1)c1cnnn1-c1ccncc1]

Acid CAS 1525417-94-0
Amine CAS 30235-29-1

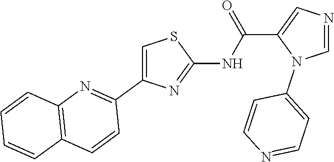 [SMILES: O=C(Nc1nc(-c2ccc3ccccc3n2)cs1)c1cncn1-c1ccncc1]

Acid CAS 1439900-49-6
Amine CAS 30235-29-1

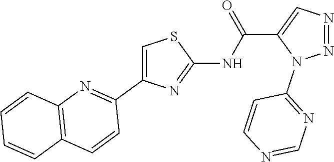 [SMILES: O=C(Nc1nc(-c2ccc3ccccc3n2)cs1)c1cnnn1-c1ccncn1]

Acid CAS 1482934-49-5
Amine CAS 30235-29-1

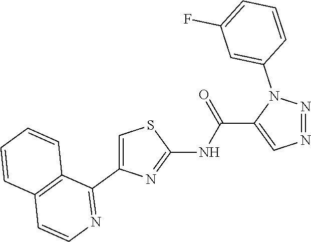 [SMILES: O=C(Nc1nc(-c2nccc3ccccc23)cs1)c1cnnn1-c1cccc(F)c1]

Acid CAS 1153490-92-6
Amine CAS 651042-73-8

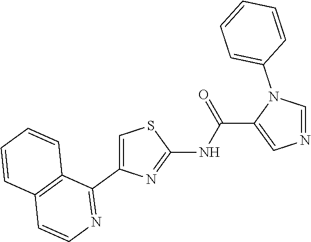 [SMILES: O=C(Nc1nc(-c2nccc3ccccc23)cs1)c1cncn1-c1ccccc1]

Acid CAS 135417-65-1
Amine CAS 651042-73-8

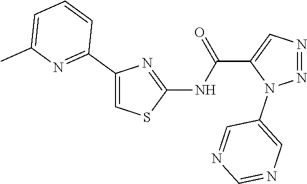 [SMILES: Cc1cccc(-c2csc(NC(=O)c3cnnn3-c3cncnc3)n2)n1]

Acid CAS 147 [symbol] 093-94-5
Amine CAS 99358-99-3

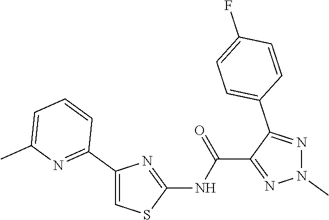

Acid CAS 483281-63-6
Amine CAS 99358-99-3

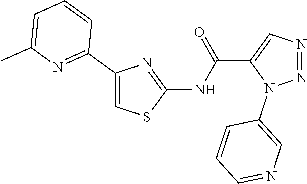 [SMILES: Cc1cccc(-c2csc(NC(=O)c3cnnn3-c3cccnc3)n2)n1]

Acid CAS 1478271-10-1
Amine CAS 99358-99-3

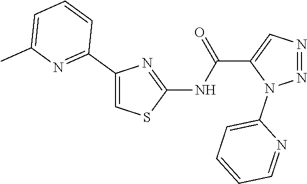 [SMILES: Cc1cccc(-c2csc(NC(=O)c3cnnn3-c3ccccn3)n2)n1]

Acid CAS 1480445-52-0
Amine CAS 99358-99-3

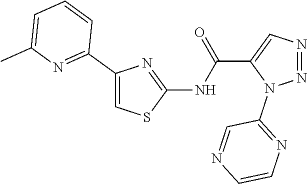 [SMILES: Cc1cccc(-c2csc(NC(=O)c3cnnn3-c3cnccn3)n2)n1]

Acid CAS 2946657-15-2
Amine CAS 99358-99-3

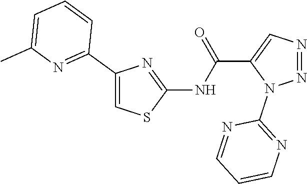 [SMILES: Cc1cccc(-c2csc(NC(=O)c3cnnn3-c3ncccn3)n2)n1]

Acid CAS 2946657-14-1
Amine CAS 99358-99-3

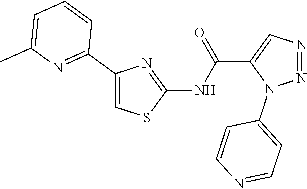 [SMILES: Cc1cccc(-c2csc(NC(=O)c3cnnn3-c3ccncc3)n2)n1]

Acid CAS 1525417-94-0
Amine CAS 99358-99-3

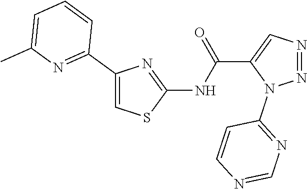 [SMILES: Cc1cccc(-c2csc(NC(=O)c3cnnn3-c3ccncn3)n2)n1]

Acid CAS 1482934-49-5
Amine CAS 99358-99-3

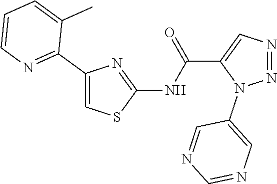 [SMILES: Cc1cccnc1-c1csc(NC(=O)c2cnnn2-c2cncnc2)n1]

Acid CAS 1478093-94-5
Amine CAS 1044269-91-1

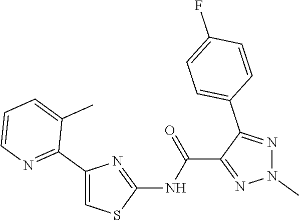 [SMILES: Cc1cccnc1-c1csc(NC(=O)c2nn(C)nc2-c2ccc(F)cc2)n1]

Acid CAS 483281-63-6
Amine CAS 1044269-91-1

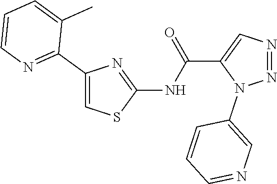 [SMILES: Cc1cccnc1-c1csc(NC(=O)c2cnnn2-c2cccnc2)n1]

Acid CAS 1478271-10-1
Amine CAS 1044269-91-1

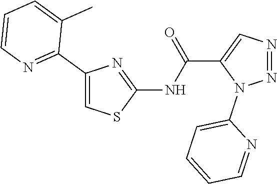 [SMILES: Cc1cccnc1-c1csc(NC(=O)c2cnnn2-c2ccccn2)n1]

Acid CAS 1480445-52-0
Amine CAS 1044269-91-1

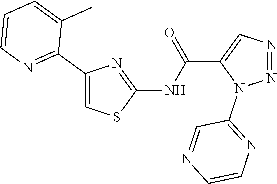 [SMILES: Cc1cccnc1-c1csc(NC(=O)c2cnnn2-c2cnccn2)n1]

Acid CAS 2946657-15-2
Amine CAS 1044269-91-1

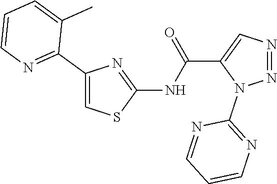 [SMILES: Cc1cccnc1-c1csc(NC(=O)c2cnnn2-c2ncccn2)n1]

Acid CAS 2946657-14-1
Amine CAS 1044269-91-1

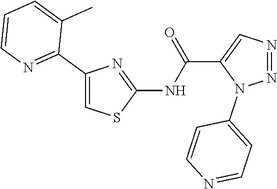 [SMILES: Cc1cccnc1-c1csc(NC(=O)c2cnnn2-c2ccncc2)n1]

Acid CAS 1525417-94-0
Amine CAS 1044269-91-1

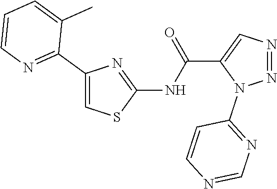 [SMILES: Cc1cccnc1-c1csc(NC(=O)c2cnnn2-c2ccncn2)n1]

Acid CAS 1482934-49-5
Amine CAS 1044269-91-1

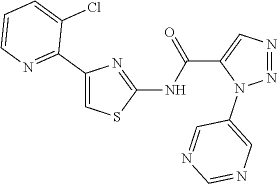 [SMILES: O=C(Nc1nc(-c2ncccc2Cl)cs1)c1cnnn1-c1cncnc1]

Acid CAS 1476093-94-5
Amine CAS 2150665-06-6

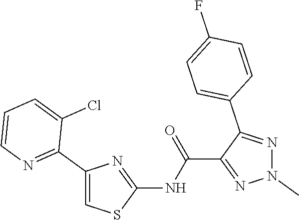 [SMILES: Cn1nc(C(=O)Nc2nc(-c3ncccc3Cl)cs2)c(-c2ccc(F)cc2)n1]

Acid CAS 483281-63-6
Amine CAS 2150665-06-6

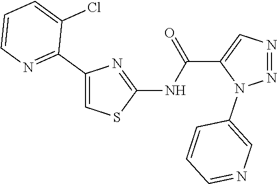 [SMILES: O=C(Nc1nc(-c2ncccc2Cl)cs1)c1cnnn1-c1cccnc1]

Acid CAS 147 [symbol] 271-10-1
Amine CAS 2150665-06-6

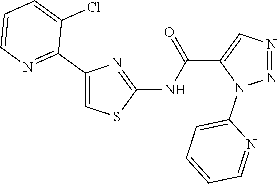 [SMILES: O=C(Nc1nc(-c2ncccc2Cl)cs1)c1cnnn1-c1ccccn1]

Acid CAS 148445-52-0
Amine CAS 2150665-06-6

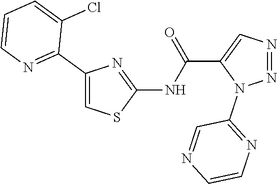 [SMILES: O=C(Nc1nc(-c2ncccc2Cl)cs1)c1cnnn1-c1cnccn1]

Acid CAS 2946657-15-2
Amine CAS 2150665-06-6

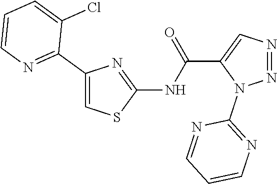 [SMILES: O=C(Nc1nc(-c2ncccc2Cl)cs1)c1cnnn1-c1ncccn1]

Acid CAS 2946657-14-1
Amine CAS 2150665-06-6

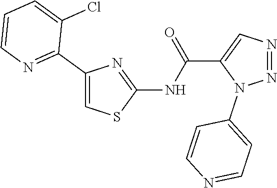 [SMILES: O=C(Nc1nc(-c2ncccc2Cl)cs1)c1cnnn1-c1ccncc1]

Acid CAS 1525417-94-0
Amine CAS 2150665-06-6

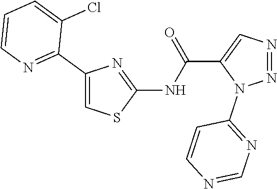 [SMILES: O=C(Nc1nc(-c2ncccc2Cl)cs1)c1cnnn1-c1ccncn1]

Acid CAS 1482934-49-5
Amine CAS 2150665-06-6

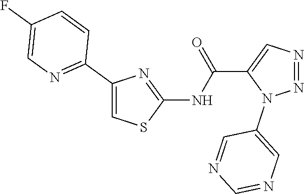 [SMILES: O=C(Nc1nc(-c2ccc(F)cn2)cs1)c1cnnn1-c1cncnc1]

Acid CAS 1478093-94-5
Amine CAS 1551651-30-9

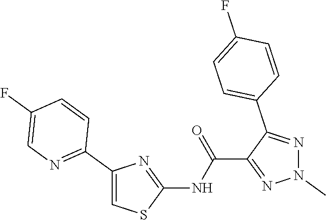 [SMILES: Cn1nc(C(=O)Nc2nc(-c3ccc(F)cn3)cs2)c(-c2ccc(F)cc2)n1]

Acid CAS 483281-63-6
Amine CAS 1551651-30-9

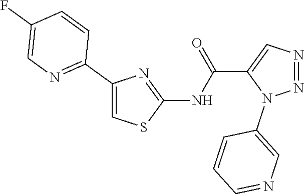 [SMILES: O=C(Nc1nc(-c2ccc(F)cn2)cs1)c1cnnn1-c1cccnc1]

Acid CAS 1478271-10-1
Amine CAS 1551651-30-9

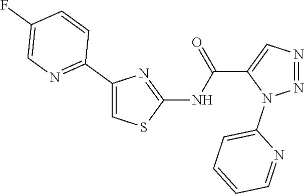 [SMILES: O=C(Nc1nc(-c2ccc(F)cn2)cs1)c1cnnn1-c1ccccn1]

Acid CAS 1480445-52-0
Amine CAS 1551651-30-9

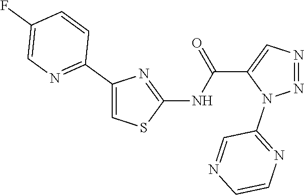 [SMILES: O=C(Nc1nc(-c2ccc(F)cn2)cs1)c1cnnn1-c1cnccn1]

Acid CAS 2946657-15-2
Amine CAS 1551651-30-9

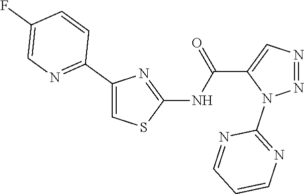 [SMILES: O=C(Nc1nc(-c2ccc(F)cn2)cs1)c1cnnn1-c1ncccn1]

Acid CAS 2946657-14-1
Amine CAS 1551651-30-9

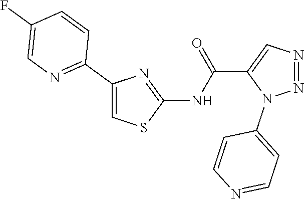 [SMILES: O=C(Nc1nc(-c2ccc(F)cn2)cs1)c1cnnn1-c1ccncc1]

Acid CAS 1525417-94-0
Amine CAS 1551651-30-9

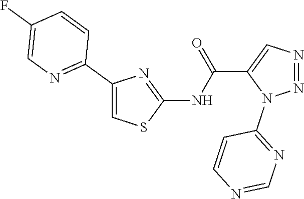 [SMILES: O=C(Nc1nc(-c2ccc(F)cn2)cs1)c1cnnn1-c1ccncn1]

Acid CAS 1462934-49-5
Amine CAS 1551651-30-9

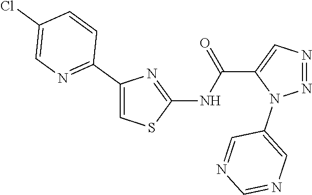 [SMILES: O=C(Nc1nc(-c2ccc(Cl)cn2)cs1)c1cnnn1-c1cncnc1]

Acid CAS 1478093-94-5
Amine CAS 1185315-17-6

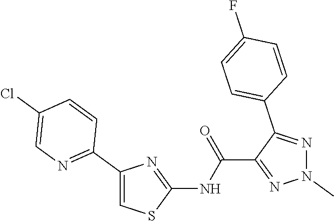 [SMILES: Cn1nc(C(=O)Nc2nc(-c3ccc(Cl)cn3)cs2)c(-c2ccc(F)cc2)n1]

Acid CAS 483281-63- [symbol]
Amine CAS 1185315-17-6

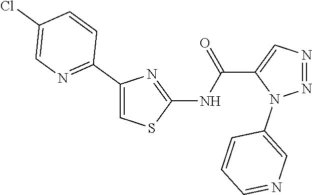 [SMILES: O=C(Nc1nc(-c2ccc(Cl)cn2)cs1)c1cnnn1-c1cccnc1]

Acid CAS 1478271-10-1
Amine CAS 1185315-17-6

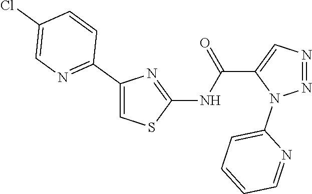 [SMILES: O=C(Nc1nc(-c2ccc(Cl)cn2)cs1)c1cnnn1-c1ccccn1]

Acid CAS 1480445-52-0
Amine CAS 1185315-17-6

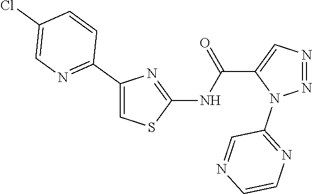 [SMILES: O=C(Nc1nc(-c2ccc(Cl)cn2)cs1)c1cnnn1-c1cnccn1]

Acid CAS 2946657-15-2
Amine CAS 1185315-17-6

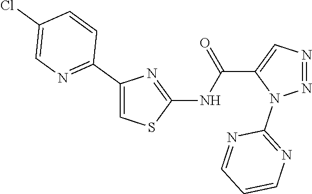 [SMILES: O=C(Nc1nc(-c2ccc(Cl)cn2)cs1)c1cnnn1-c1ncccn1]

Acid CAS 2946657-14-1
Amine CAS 1185315-17-6

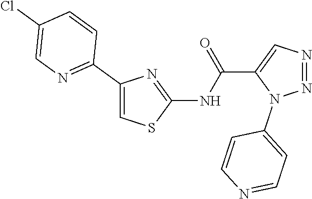 [SMILES: O=C(Nc1nc(-c2ccc(Cl)cn2)cs1)c1cnnn1-c1ccncc1]

Acid CAS 1525417-94-0
Amine CAS 1185315-17-6

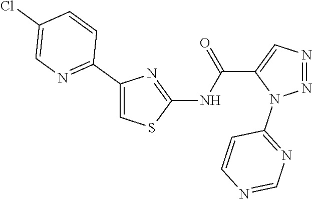 [SMILES: O=C(Nc1nc(-c2ccc(Cl)cn2)cs1)c1cnnn1-c1ccncn1]

Acid CAS 1482934-49-5
Amine CAS 1185315-17-6

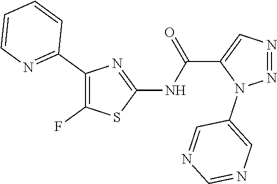 [SMILES: O=C(Nc1nc(-c2ccccn2)c(F)s1)c1cnnn1-c1cncnc1]

Acid CAS 1478093-94-5
Amine CAS 1044270-93-0

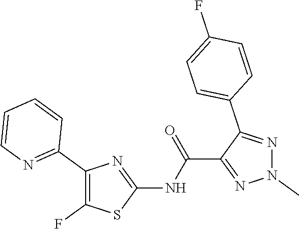 [SMILES: Cn1nc(C(=O)Nc2nc(-c3ccccn3)c(F)s2)c(-c2ccc(F)cc2)n1]

Acid CAS 483281-63-6
Amine CAS 1044270-93-0

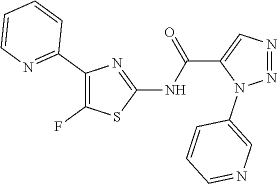 [SMILES: O=C(Nc1nc(-c2ccccn2)c(F)s1)c1cnnn1-c1cccnc1]

Acid CAS 1478271-10-1
Amine CAS 1044270-93-0

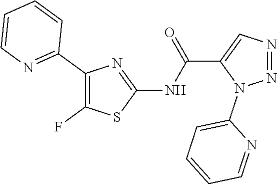 [SMILES: O=C(Nc1nc(-c2ccccn2)c(F)s1)c1cnnn1-c1ccccn1]

Acid CAS 1480445-52-0
Amine CAS 1044270-93-0

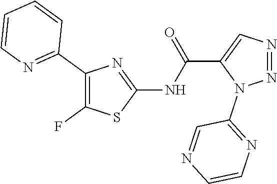 [SMILES: O=C(Nc1nc(-c2ccccn2)c(F)s1)c1cnnn1-c1cnccn1]

Acid CAS 2946657-15-2
Amine CAS 1044270-93-0

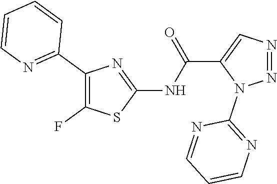 [SMILES: O=C(Nc1nc(-c2ccccn2)c(F)s1)c1cnnn1-c1ncccn1]

Acid CAS 2946657-14-1
Amine CAS 1044270-93-0

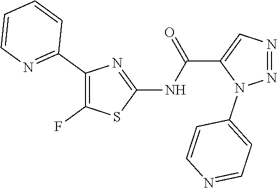 [SMILES: O=C(Nc1nc(-c2ccccn2)c(F)s1)c1cnnn1-c1ccncc1]

Acid CAS 1525417-94-0
Amine CAS 1044270-93-0

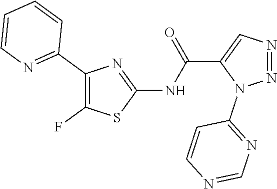 [SMILES: O=C(Nc1nc(-c2ccccn2)c(F)s1)c1cnnn1-c1ccncn1]

Acid CAS 1482934-49-5
Amine CAS 1044270-93-0

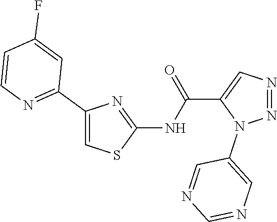 [SMILES: O=C(Nc1nc(-c2cc(F)ccn2)cs1)c1cnnn1-c1cncnc1]

Acid CAS 147 [symbol] 093-94-5
Amine CAS 2228222-84-0

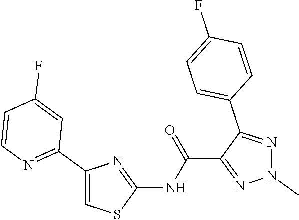 [SMILES: Cn1nc(C(=O)Nc2nc(-c3cc(F)ccn3)cs2)c(-c2ccc(F)cc2)n1]

Acid CAS 483281-63-6
Amine CAS 2228222-84-0

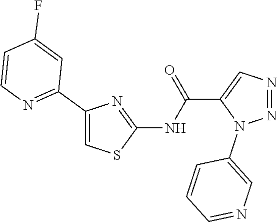 [SMILES: O=C(Nc1nc(-c2cc(F)ccn2)cs1)c1cnnn1-c1cccnc1]

Acid CAS 147 [symbol] 271-10-1
Amine CAS 2228222-84-0

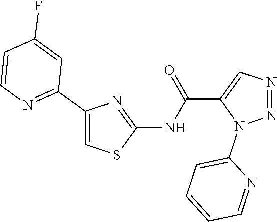 [SMILES: O=C(Nc1nc(-c2cc(F)ccn2)cs1)c1cnnn1-c1ccccn1]

Acid CAS 14 [symbol] 0445-52-0
Amine CAS 2228222-84-0

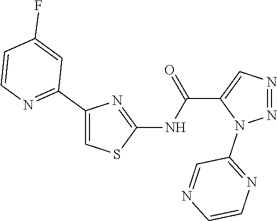 [SMILES: O=C(Nc1nc(-c2cc(F)ccn2)cs1)c1cnnn1-c1cnccn1]

Acid CAS 2946657-15-2
Amine CAS 2228222-84-0

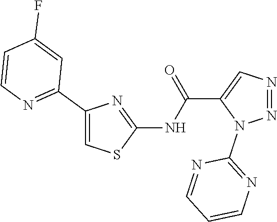 [SMILES: O=C(Nc1nc(-c2cc(F)ccn2)cs1)c1cnnn1-c1ncccn1]

Acid CAS 2946657-14-1
Amine CAS 2228222-84-0

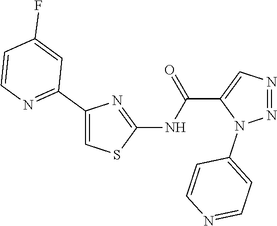 [SMILES: O=C(Nc1nc(-c2cc(F)ccn2)cs1)c1cnnn1-c1ccncc1]

Acid CAS 1525417-94-0
Amine CAS 2228222-84-0

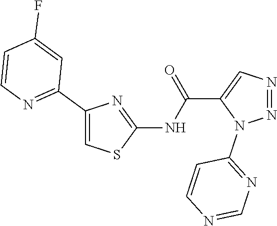 [SMILES: O=C(Nc1nc(-c2cc(F)ccn2)cs1)c1cnnn1-c1ccncn1]

Acid CAS 148934-49-5
Amine CAS 2228222-84-0

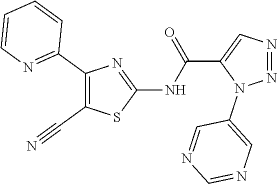 [SMILES: N#Cc1sc(NC(=O)c2cnnn2-c2cncnc2)nc1-c1ccccn1]

Acid CAS 1478093-94-5
Amine CAS 1139743-06-8

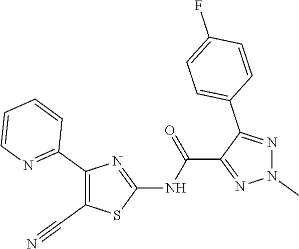 [SMILES: Cn1nc(C(=O)Nc2nc(-c3ccccn3)c(C#N)s2)c(-c2ccc(F)cc2)n1]

Acid CAS 483281-63-6
Amine CAS 1139743-06-8

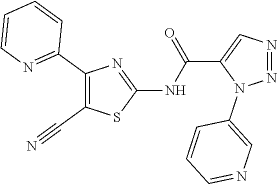 [SMILES: N#Cc1sc(NC(=O)c2cnnn2-c2cccnc2)nc1-c1ccccn1]

Acid CAS 1478271-10-1
Amine CAS 1139743-06-8

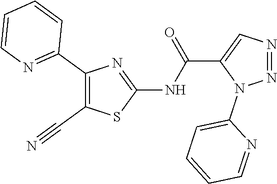 [SMILES: N#Cc1sc(NC(=O)c2cnnn2-c2ccccn2)nc1-c1ccccn1]

Acid CAS 1480445-52-0
Amine CAS 1139743-06-8

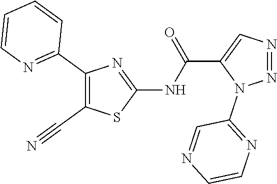 [SMILES: N#Cc1sc(NC(=O)c2cnnn2-c2cnccn2)nc1-c1ccccn1]

Acid CAS 2946657-15-2
Amine CAS 1139743-06-8

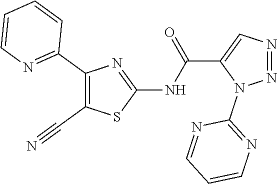 [SMILES: N#Cc1sc(NC(=O)c2cnnn2-c2ncccn2)nc1-c1ccccn1]

Acid CAS 2946657-14-1
Amine CAS1139743-06-8

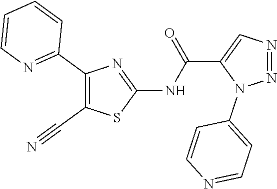 [SMILES: N#Cc1sc(NC(=O)c2cnnn2-c2ccncc2)nc1-c1ccccn1]

Acid CAS 1525417-94-0
Amine CAS 1139743-06-8

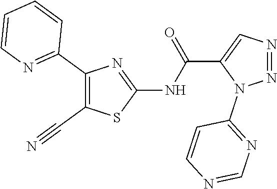 [SMILES: N#Cc1sc(NC(=O)c2cnnn2-c2ccncn2)nc1-c1ccccn1]

Acid CAS 1482934-49-5
Amine CAS 1139743-06-8

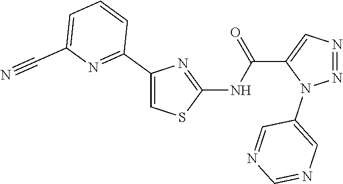 [SMILES: N#Cc1cccc(-c2csc(NC(=O)c3cnnn3-c3cncnc3)n2)n1]

Acid CAS 1478093-94-5
Amine CAS 1185317-90-1

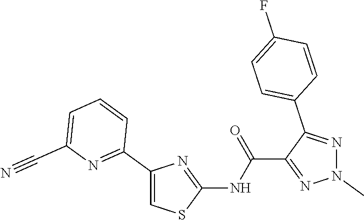 [SMILES: Cn1nc(C(=O)Nc2nc(-c3cccc(C#N)n3)cs2)c(-c2ccc(F)cc2)n1]

Acid CAS 483281-63-6
Amine CAS 1185317-90-1

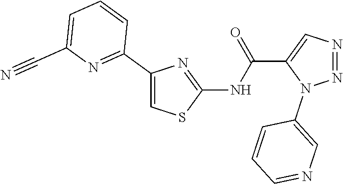 [SMILES: N#Cc1cccc(-c2csc(NC(=O)c3cnnn3-c3cccnc3)n2)n1]

Acid CAS 1478271-10-1
Amine CAS 1185317-90-1

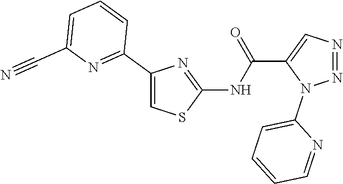 [SMILES: N#Cc1cccc(-c2csc(NC(=O)c3cnnn3-c3ccccn3)n2)n1]

Acid CAS 11480445-52-0
Amine CAS 1185317-90-1

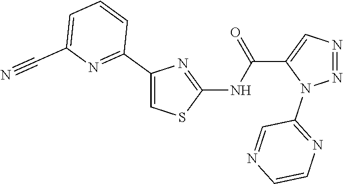 [SMILES: N#Cc1cccc(-c2csc(NC(=O)c3cnnn3-c3cnccn3)n2)n1]

Acid CAS 2946657-15-2
Amine CAS 1185317-90-1

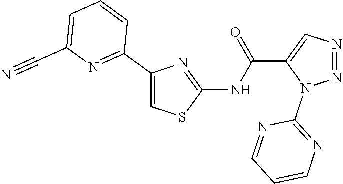 [SMILES: N#Cc1cccc(-c2csc(NC(=O)c3cnnn3-c3ncccn3)n2)n1]

Acid CAS 2946657-14-1
Amine CAS 1185317-90-1

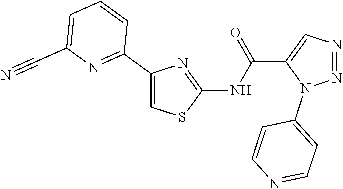 [SMILES: N#Cc1cccc(-c2csc(NC(=O)c3cnnn3-c3ccncc3)n2)n1]

Acid CAS 1525417-94-0
Amine CAS 1185317-90-1

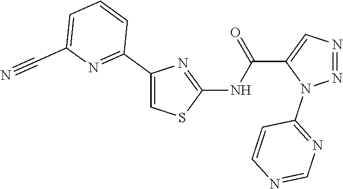 [SMILES: N#Cc1cccc(-c2csc(NC(=O)c3cnnn3-c3ccncn3)n2)n1]

Acid CAS 1482934-49-5
Amine CAS 1185317-90-1

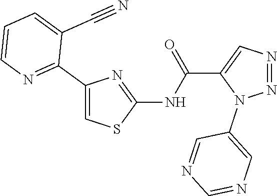 [SMILES: N#Cc1cccnc1-c1csc(NC(=O)c2cnnn2-c2cncnc2)n1]

Acid CAS 1478093-94-5
Amine CAS 2253868-93-6

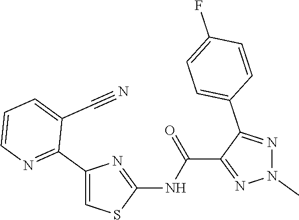 [SMILES: Cn1nc(C(=O)Nc2nc(-c3ncccc3C#N)cs2)c(-c2ccc(F)cc2)n1]

Acid CAS 483281-63-6
Amine CAS 2253868-93-6

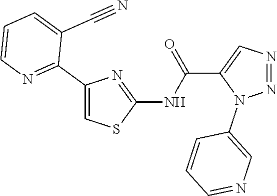 [SMILES: N#Cc1cccnc1-c1csc(NC(=O)c2cnnn2-c2cccnc2)n1]

Acid CAS 1478271-10-1
Amine CAS 2253868-93-6

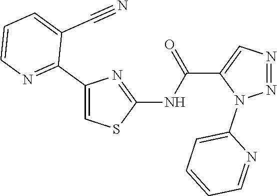 [SMILES: N#Cc1cccnc1-c1csc(NC(=O)c2cnnn2-c2ccccn2)n1]

Acid CAS 1480445-52-0
Amine CAS 2253868-93-6

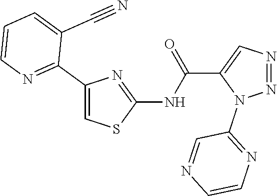 [SMILES: N#Cc1cccnc1-c1csc(NC(=O)c2cnnn2-c2cnccn2)n1]

Acid CAS 2946657-15-2
Amine CAS 2253868-93-6

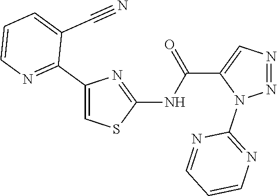 [SMILES: N#Cc1cccnc1-c1csc(NC(=O)c2cnnn2-c2ncccn2)n1]

Acid CAS 2946657-14-1
Amine CAS 2253868-93-6

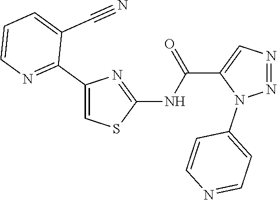 [SMILES: N#Cc1cccnc1-c1csc(NC(=O)c2cnnn2-c2ccncc2)n1]

Acid CAS 1525417-94-0
Amine CAS 2253868-93-6

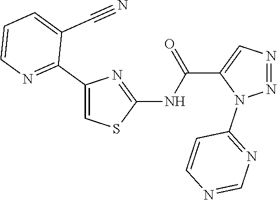 [SMILES: N#Cc1cccnc1-c1csc(NC(=O)c2cnnn2-c2ccncn2)n1]

Acid CAS 1482934-49-5
Amine CAS 2253868-93-6

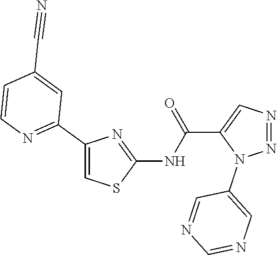 [SMILES: N#Cc1ccnc(-c2csc(NC(=O)c3cnnn3-c3cncnc3)n2)c1]

Acid CAS 1478093-94-5
Amine CAS 2253868-97-0

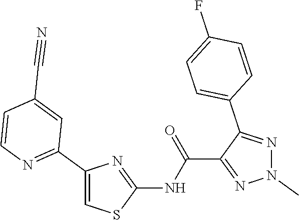 [SMILES: Cn1nc(C(=O)Nc2nc(-c3cc(C#N)ccn3)cs2)c(-c2ccc(F)cc2)n1]

Acid CAS 483281-63-6
Amine CAS 2253868-97-0

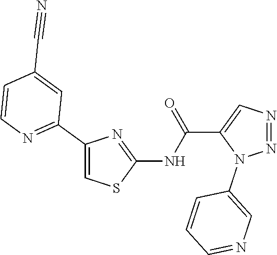 [SMILES: N#Cc1ccnc(-c2csc(NC(=O)c3cnnn3-c3cccnc3)n2)c1]

Acid CAS 1478271-10-1
Amine CAS 2253868-97-0

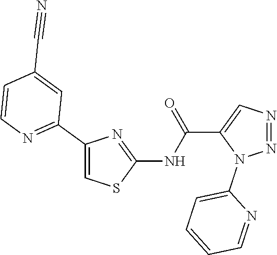 [SMILES: N#Cc1ccnc(-c2csc(NC(=O)c3cnnn3-c3ccccn3)n2)c1]

Acid CAS 1480445-52-0
Amine CAS 2253868-97-0

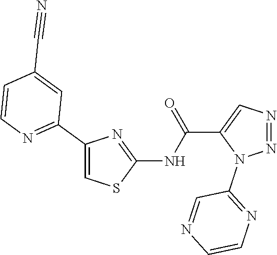 [SMILES: N#Cc1ccnc(-c2csc(NC(=O)c3cnnn3-c3cnccn3)n2)c1]

Acid CAS 2946657-15-2
Amine CAS 2253868-97-0

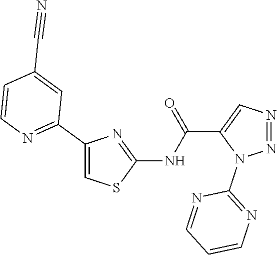 [SMILES: N#Cc1ccnc(-c2csc(NC(=O)c3cnnn3-c3ncccn3)n2)c1]

Acid CAS 2946657-14-1
Amine CAS 2253868-97-0

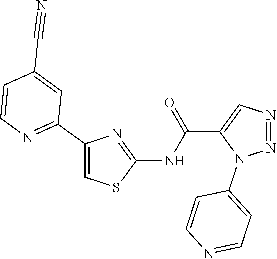 [SMILES: N#Cc1ccnc(-c2csc(NC(=O)c3cnnn3-c3ccncc3)n2)c1]

Acid CAS 1525417-94-0
Amine CAS 2253868-97-0

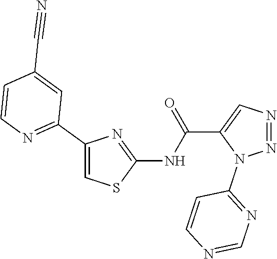 [SMILES: N#Cc1ccnc(-c2csc(NC(=O)c3cnnn3-c3ccncn3)n2)c1]

Acid CAS 1482934-49-5
Amine CAS 2253868-97-0

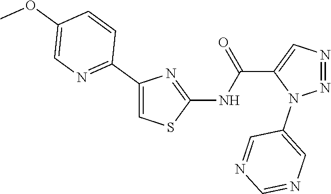 [SMILES: COc1ccc(-c2csc(NC(=O)c3cnnn3-c3cncnc3)n2)nc1]

Acid CAS 1478093-94-5
Amine CAS 228346-85-6

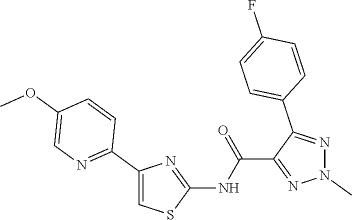

Acid CAS 483281-63-6
Amine CAS 2228346-85-6

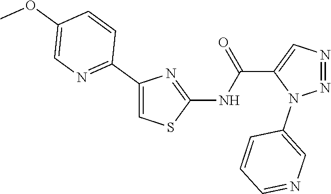 [SMILES: COc1ccc(-c2csc(NC(=O)c3cnnn3-c3cccnc3)n2)nc1]

Acid CAS 1478271-10-1
Amine CAS 2228346-85-6

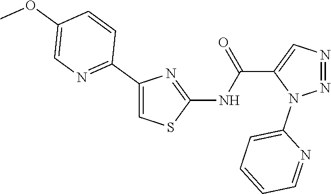 [SMILES: COc1ccc(-c2csc(NC(=O)c3cnnn3-c3ccccn3)n2)nc1]

Acid CAS 1480445-52-0
Amine CAS 2228346-85-0

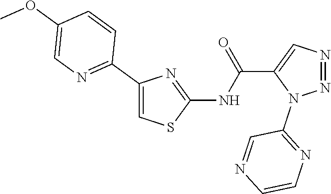 [SMILES: COc1ccc(-c2csc(NC(=O)c3cnnn3-c3cnccn3)n2)nc1]

Acid CAS 2946657-15-2
Amine CAS 2228346-85-6

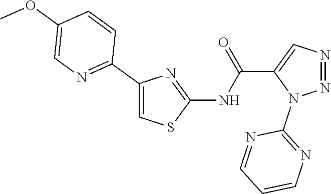 [SMILES: COc1ccc(-c2csc(NC(=O)c3cnnn3-c3ncccn3)n2)nc1]

Acid CAS 2946657-14-1
Amine CAS 2228346-85-6

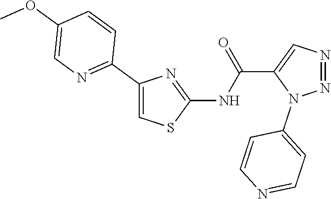 [SMILES: COc1ccc(-c2csc(NC(=O)c3cnnn3-c3ccncc3)n2)nc1]

Acid CAS 1525417-94-0
Amine CAS 2228346-85-6

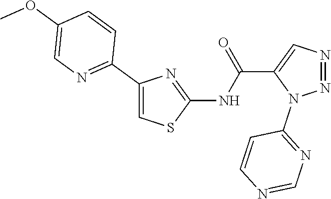 [SMILES: COc1ccc(-c2csc(NC(=O)c3cnnn3-c3ccncn3)n2)nc1]

Acid CAS 1482934-49-5
Amine CAS 2228346-85-0

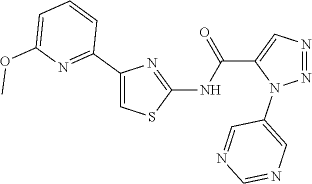 [SMILES: COc1cccc(-c2csc(NC(=O)c3cnnn3-c3cncnc3)n2)n1]

Acid CAS 1478093-94-5
Amine CAS 1870028-77-5

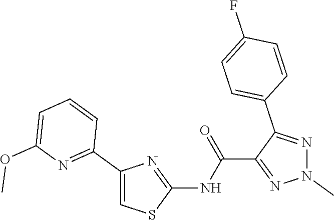

Acid CAS 483281-63-6
Amine CAS 1870028-77-5

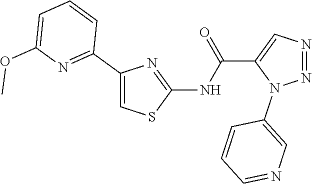 [SMILES: COc1cccc(-c2csc(NC(=O)c3cnnn3-c3cccnc3)n2)n1]

Acid CAS 147 [symbol] 271-10-1
Amine CAS 1870028-77-5

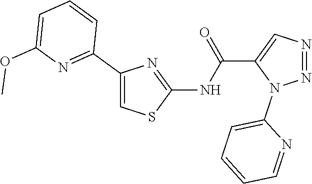 [SMILES: COc1cccc(-c2csc(NC(=O)c3cnnn3-c3ccccn3)n2)n1]

Acid CAS 1480445-52-0
Amine CAS 1870028-77-5

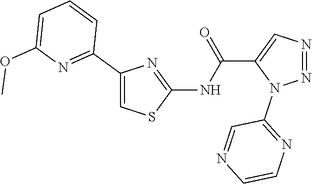 [SMILES: COc1cccc(-c2csc(NC(=O)c3cnnn3-c3cnccn3)n2)n1]

Acid CAS 2946657-15-2
Amine CAS 1870028-77-5

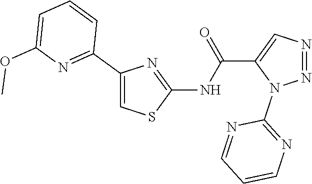 [SMILES: COc1cccc(-c2csc(NC(=O)c3cnnn3-c3ncccn3)n2)n1]

Acid CAS 2946657-14-1
Amine CAS 1870028-77-5

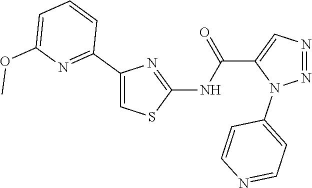 [SMILES: COc1cccc(-c2csc(NC(=O)c3cnnn3-c3ccncc3)n2)n1]

Acid CAS 1525417-94-0
Amine CAS 1870028-77-5

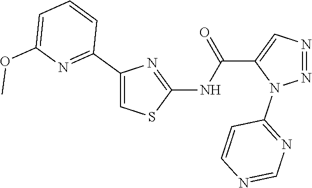 [SMILES: COc1cccc(-c2csc(NC(=O)c3cnnn3-c3ccncn3)n2)n1]

Acid CAS 1482934-49-5
Amine CAS 1870028-77-5

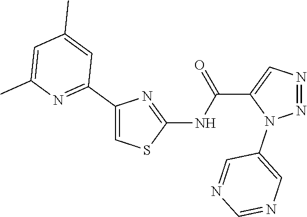 [SMILES: Cc1cc(C)nc(-c2csc(NC(=O)c3cnnn3-c3cncnc3)n2)c1]

Acid CAS 1478093-94-5
Amine CAS 1865056-88-7

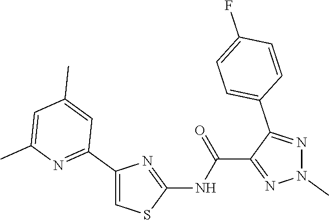

Acid CAS 483281-63-6
Amine CAS 1865056-88-7

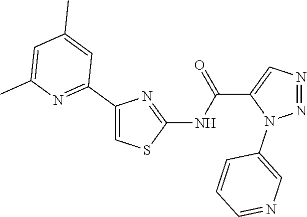 [SMILES: Cc1cc(C)nc(-c2csc(NC(=O)c3cnnn3-c3cccnc3)n2)c1]

Acid CAS 147 [symbol] 271-10-1
Amine CAS 1865058-88-7

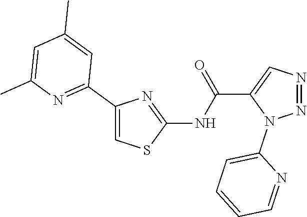 [SMILES: Cc1cc(C)nc(-c2csc(NC(=O)c3cnnn3-c3ccccn3)n2)c1]

Acid CAS 1480445-52-0
Amine CAS 1865056-88-7

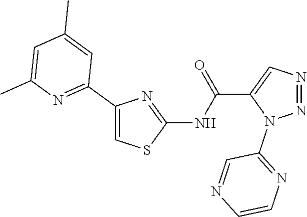 [SMILES: Cc1cc(C)nc(-c2csc(NC(=O)c3cnnn3-c3cnccn3)n2)c1]

Acid CAS 2946657-15-2
Amine CAS 1865056-88-7

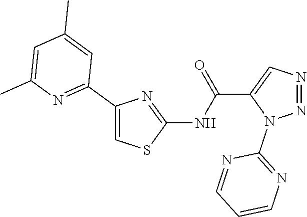 [SMILES: Cc1cc(C)nc(-c2csc(NC(=O)c3cnnn3-c3ncccn3)n2)c1]

Acid CAS 2946657-14-1
Amine CAS 1865056-88-7

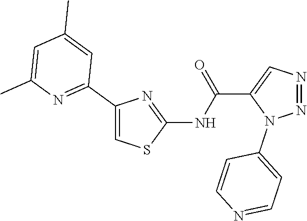 [SMILES: Cc1cc(C)nc(-c2csc(NC(=O)c3cnnn3-c3ccncc3)n2)c1]

Acid CAS 1525417-94-0
Amine CAS 1865056-88-7

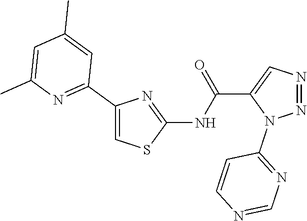 [SMILES: Cc1cc(C)nc(-c2csc(NC(=O)c3cnnn3-c3ccncn3)n2)c1]

Acid CAS 1482934-49-5
Amine CAS 1865056-88-7

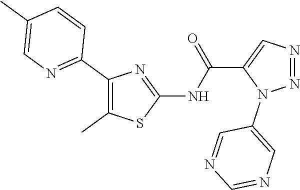 [SMILES: Cc1ccc(-c2nc(NC(=O)c3cnnn3-c3cncnc3)sc2C)nc1]

Acid CAS 1478093-94-5
Amine CAS 2294596-48-6

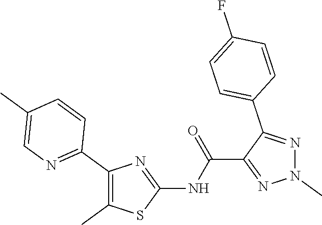

Acid CAS 483281-63-6
Amine CAS 2294596-48-6

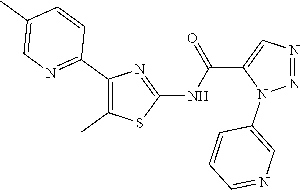 [SMILES: Cc1ccc(-c2nc(NC(=O)c3cnnn3-c3cccnc3)sc2C)nc1]

Acid CAS 1478271-10-1
Amine CAS 2294596-48-6

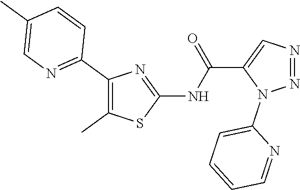 [SMILES: Cc1ccc(-c2nc(NC(=O)c3cnnn3-c3ccccn3)sc2C)nc1]

Acid CAS 1480445-52-0
Amine CAS 2294596-48-6

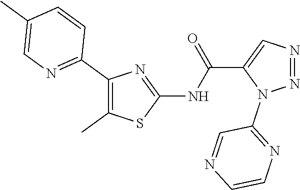 [SMILES: Cc1ccc(-c2nc(NC(=O)c3cnnn3-c3cnccn3)sc2C)nc1]

Acid CAS 2946657-15-2
Amine CAS 2294596-48-6

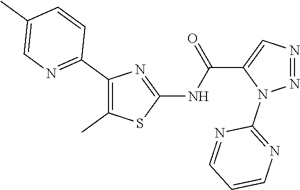 [SMILES: Cc1ccc(-c2nc(NC(=O)c3cnnn3-c3ncccn3)sc2C)nc1]

Acid CAS 2946657-14-1
Amine CAS 2294596-48-6

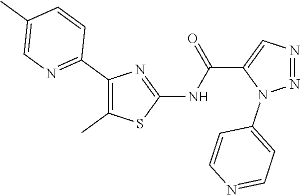 [SMILES: Cc1ccc(-c2nc(NC(=O)c3cnnn3-c3ccncc3)sc2C)nc1]

Acid CAS 1525417-94-0
Amine CAS 2294596-48-6

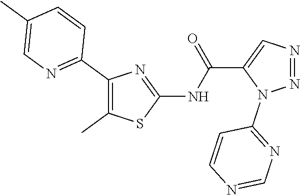 [SMILES: Cc1ccc(-c2nc(NC(=O)c3cnnn3-c3ccncn3)sc2C)nc1]

Acid CAS 1482934-49-5
Amine CAS 2294596-48-6

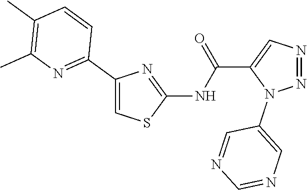 [SMILES: Cc1ccc(-c2csc(NC(=O)c3cnnn3-c3cncnc3)n2)nc1C]

Acid CAS 1476093-94-5
Amine CAS 1934459-93-4

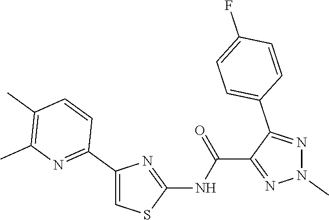

Acid CAS 483281-63-6
Amine CAS 1934459-93-4

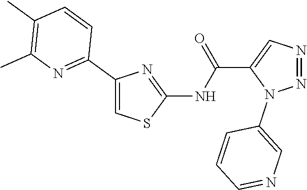 [SMILES: Cc1ccc(-c2csc(NC(=O)c3cnnn3-c3cccnc3)n2)nc1C]

Acid CAS 1478271-10-1
Amine CAS 1934459-93-4

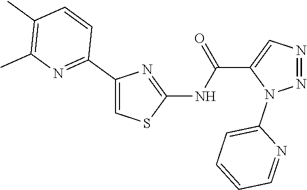 [SMILES: Cc1ccc(-c2csc(NC(=O)c3cnnn3-c3ccccn3)n2)nc1C]

Acid CAS 1480445-52-0
Amine CAS 1934459-93-4

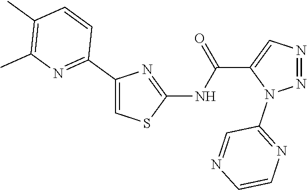 [SMILES: Cc1ccc(-c2csc(NC(=O)c3cnnn3-c3cnccn3)n2)nc1C]

Acid CAS 2946657-15-2
Amine CAS 1934459-93-4

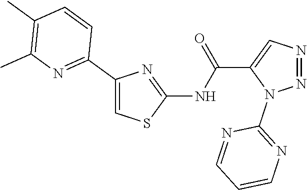 [SMILES: Cc1ccc(-c2csc(NC(=O)c3cnnn3-c3ncccn3)n2)nc1C]

Acid CAS 2946657-14-1
Amine CAS 1934459-93-4

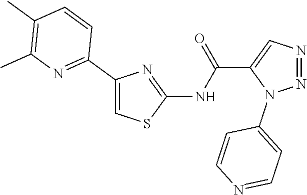 [SMILES: Cc1ccc(-c2csc(NC(=O)c3cnnn3-c3ccncc3)n2)nc1C]

Acid CAS 1525417-94-0
Amine CAS 1934459-93-4

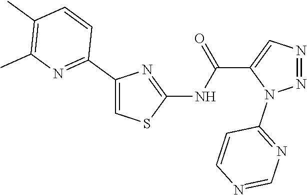 [SMILES: Cc1ccc(-c2csc(NC(=O)c3cnnn3-c3ccncn3)n2)nc1C]

Acid CAS 1482934-49-5
Amine CAS 1934459-93-4

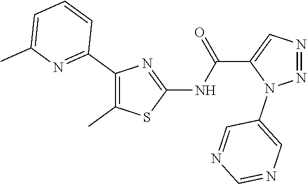 [SMILES: Cc1cccc(-c2nc(NC(=O)c3cnnn3-c3cncnc3)sc2C)n1]

Acid CAS 1478093-94-5
Amine CAS 2169472-77-7

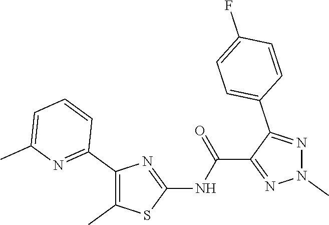

Acid CAS 483281-63-6
Amine CAS 2169472-77-7

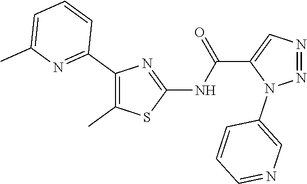 [SMILES: Cc1cccc(-c2nc(NC(=O)c3cnnn3-c3cccnc3)sc2C)n1]

Acid CAS 1478271-10-1
Amine CAS 2169472-77-7

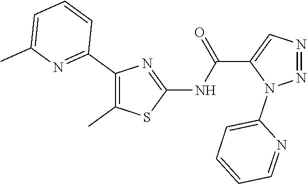 [SMILES: Cc1cccc(-c2nc(NC(=O)c3cnnn3-c3ccccn3)sc2C)n1]

Acid CAS 1480445-52-0
Amine CAS 2169472-77-7

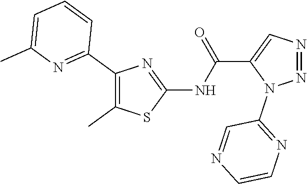 [SMILES: Cc1cccc(-c2nc(NC(=O)c3cnnn3-c3cnccn3)sc2C)n1]

Acid CAS 2946657-15-2
Amine CAS 2169472-77-7

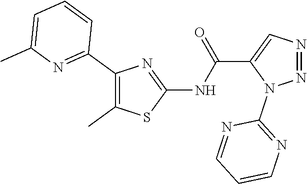 [SMILES: Cc1cccc(-c2nc(NC(=O)c3cnnn3-c3ncccn3)sc2C)n1]

Acid CAS 2946657-14-1
Amine CAS 2169472-77-7

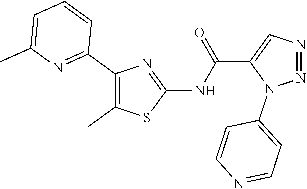 [SMILES: Cc1cccc(-c2nc(NC(=O)c3cnnn3-c3ccncc3)sc2C)n1]

Acid CAS 1525417-94-0
Amine CAS 2169472-77-7

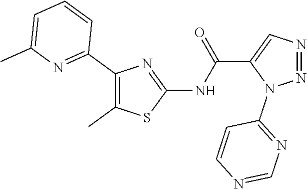 [SMILES: Cc1cccc(-c2nc(NC(=O)c3cnnn3-c3ccncn3)sc2C)n1]

Acid CAS 1482934-49-5
Amine CAS 2169472-77-7

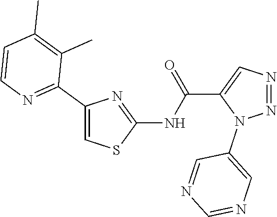 [SMILES: Cc1ccnc(-c2csc(NC(=O)c3cnnn3-c3cncnc3)n2)c1C]

Acid CAS 1478093-94-5
Amine CAS 2169166-95-2

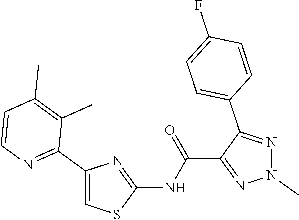 [SMILES: Cc1ccnc(-c2csc(NC(=O)c3nn(C)nc3-c3ccc(F)cc3)n2)c1C]

Acid CAS 483281-63-6
Amine CAS 2169166-95-2

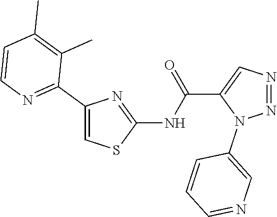 [SMILES: Cc1ccnc(-c2csc(NC(=O)c3cnnn3-c3cccnc3)n2)c1C]

Acid CAS 1478271-10-1
Amine CAS 2169166-95-2

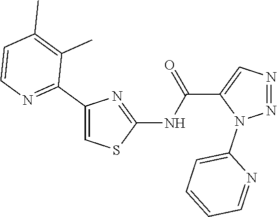 [SMILES: Cc1ccnc(-c2csc(NC(=O)c3cnnn3-c3ccccn3)n2)c1C]

Acid CAS 1480445-52-0
Amine CAS 2169166-95-2

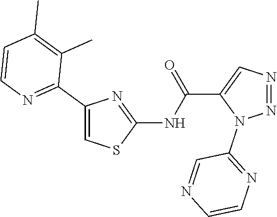 [SMILES: Cc1ccnc(-c2csc(NC(=O)c3cnnn3-c3cnccn3)n2)c1C]

Acid CAS 2946657-15-2
Amine CAS 2169166-95-2

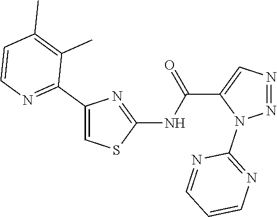 [SMILES: Cc1ccnc(-c2csc(NC(=O)c3cnnn3-c3ncccn3)n2)c1C]

Acid CAS 2946657-14-1
Amine CAS 2169166-95-2

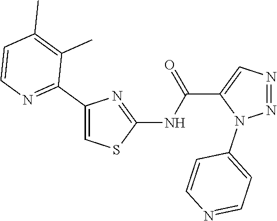 [SMILES: Cc1ccnc(-c2csc(NC(=O)c3cnnn3-c3ccncc3)n2)c1C]

Acid CAS 1525417-94-0
Amine CAS 2169166-95-2

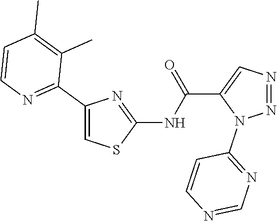 [SMILES: Cc1ccnc(-c2csc(NC(=O)c3cnnn3-c3ccncn3)n2)c1C]

Acid CAS 1482934-49-5
Amine CAS 2169168-95-2

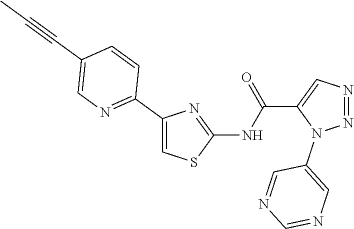 [SMILES: CC#Cc1ccc(-c2csc(NC(=O)c3cnnn3-c3cncnc3)n2)nc1]

Acid CAS 1478093-94-5
Amine CAS 2592824-00-3

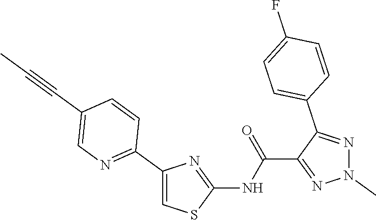 [SMILES: CC#Cc1ccc(-c2csc(NC(=O)c3nn(C)nc3-c3ccc(F)cc3)n2)nc1]

Acid CAS 483281-63-6
Amine CAS 2592824-00-3

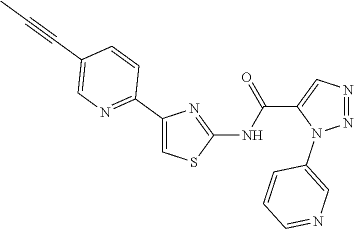 [SMILES: CC#Cc1ccc(-c2csc(NC(=O)c3cnnn3-c3cccnc3)n2)nc1]

Acid CAS 1478271-10-1
Amine CAS 2592824-00-3

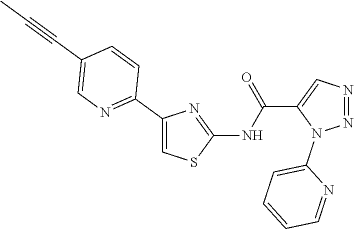 [SMILES: CC#Cc1ccc(-c2csc(NC(=O)c3cnnn3-c3ccccn3)n2)nc1]

Acid CAS 1480445-52-0
Amine CAS 2592824-00-3

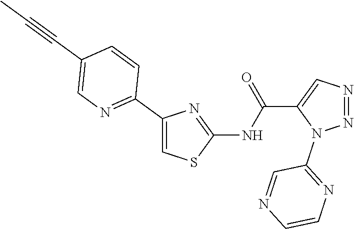 [SMILES: CC#Cc1ccc(-c2csc(NC(=O)c3cnnn3-c3cnccn3)n2)nc1]

Acid CAS 2946657-15-2
Amine CAS 2592824-00-3

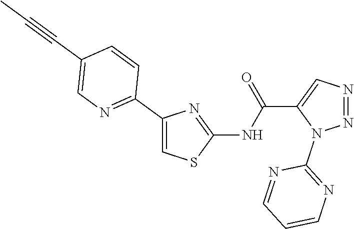 [SMILES: CC#Cc1ccc(-c2csc(NC(=O)c3cnnn3-c3ncccn3)n2)nc1]

Acid CAS 2946657-14-1
Amine CAS 2592824-00-3

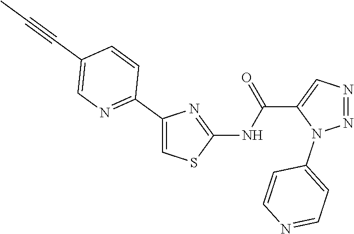 [SMILES: CC#Cc1ccc(-c2csc(NC(=O)c3cnnn3-c3ccncc3)n2)nc1]

Acid CAS 1525417-94-0
Amine CAS 2592824-00-3

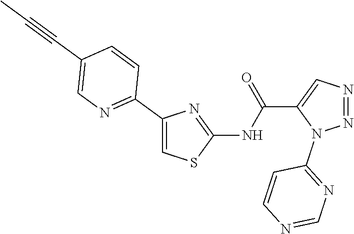 [SMILES: CC#Cc1ccc(-c2csc(NC(=O)c3cnnn3-c3ccncn3)n2)nc1]

Acid CAS 1482934-49-5
Amine CAS 2592824-00-3

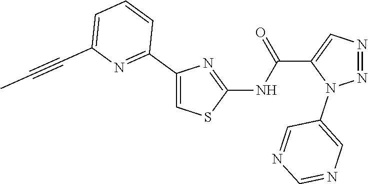 [SMILES: CC#Cc1cccc(-c2csc(NC(=O)c3cnnn3-c3cncnc3)n2)n1]

Acid CAS 1478093-94-5
Amine CAS 2600157-36-4

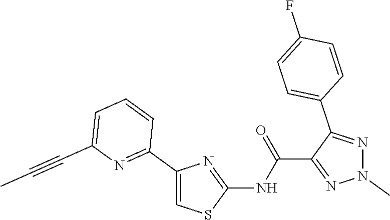 [SMILES: CC#Cc1cccc(-c2csc(NC(=O)c3nn(C)nc3-c3ccc(F)cc3)n2)n1]

Acid CAS 483281-63-6
Amine CAS 2600157-36-4

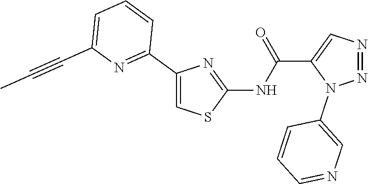 [SMILES: CC#Cc1cccc(-c2csc(NC(=O)c3cnnn3-c3cccnc3)n2)n1]

Acid CAS 1478271-10-1
Amine CAS 2600157-36-4

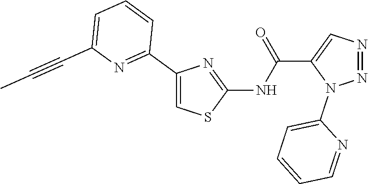 [SMILES: CC#Cc1cccc(-c2csc(NC(=O)c3cnnn3-c3ccccn3)n2)n1]

Acid CAS 1480445-52-0
Amine CAS 2600157-36-4

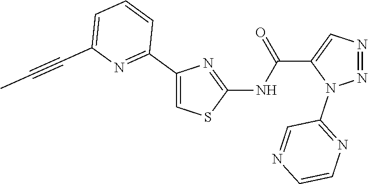 [SMILES: CC#Cc1cccc(-c2csc(NC(=O)c3cnnn3-c3cnccn3)n2)n1]

Acid CAS 2946657-15-2
Amine CAS 2600157-36-4

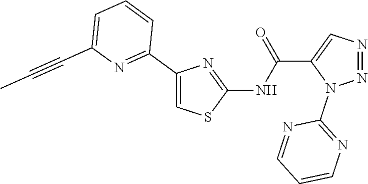 [SMILES: CC#Cc1cccc(-c2csc(NC(=O)c3cnnn3-c3ncccn3)n2)n1]

Acid CAS 2946657-14-1
Amine CAS 2600157-36-4

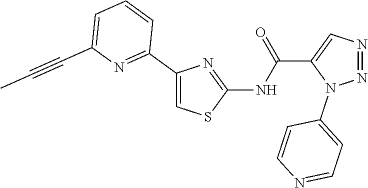 [SMILES: CC#Cc1cccc(-c2csc(NC(=O)c3cnnn3-c3ccncc3)n2)n1]

Acid CAS 1525417-94-0
Amine CAS 2600157-36-4

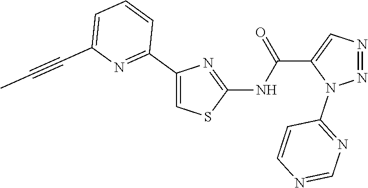 [SMILES: CC#Cc1cccc(-c2csc(NC(=O)c3cnnn3-c3ccncn3)n2)n1]

Acid CAS 1482934-49-5
Amine CAS 2600157-36-4

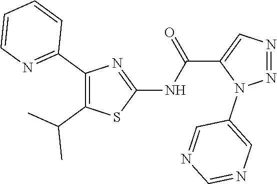 [SMILES: CC(C)c1sc(NC(=O)c2cnnn2-c2cncnc2)nc1-c1ccccn1]

Acid CAS 1478093-94-5
Amine CAS 1225774-92-4

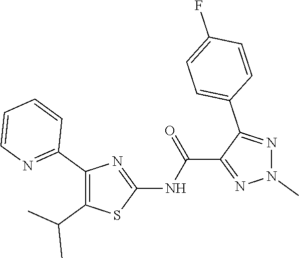 [SMILES: CC(C)c1sc(NC(=O)c2nn(C)nc2-c2ccc(F)cc2)nc1-c1ccccn1]

Acid CAS 4 [symbol] 3281-63-6
Amine CAS 1225774-92-4

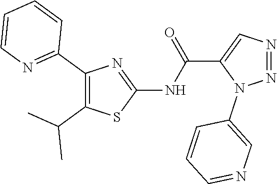 [SMILES: CC(C)c1sc(NC(=O)c2cnnn2-c2cccnc2)nc1-c1ccccn1]

Acid CAS 1478271-10-1
Amine CAS 1225774-92-4

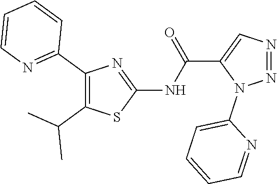 [SMILES: CC(C)c1sc(NC(=O)c2cnnn2-c2ccccn2)nc1-c1ccccn1]

Acid CAS 14 [symbol] 0445-52-0
Amine CAS 1225774-92-4

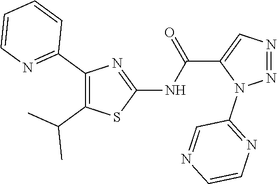 [SMILES: CC(C)c1sc(NC(=O)c2cnnn2-c2cnccn2)nc1-c1ccccn1]

Acid CAS 2946657-15-2
Amine CAS 1225774-92-4

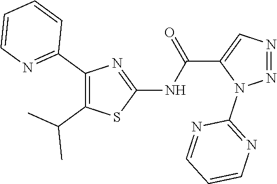 [SMILES: CC(C)c1sc(NC(=O)c2cnnn2-c2ncccn2)nc1-c1ccccn1]

Acid CAS 2946657-14-1
Amine CAS 1225774-92-4

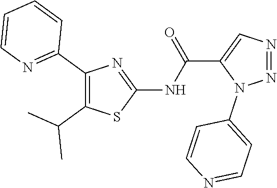 [SMILES: CC(C)c1sc(NC(=O)c2cnnn2-c2ccncc2)nc1-c1ccccn1]

Acid CAS 1525417-94-0
Amine CAS 1225774-92-4

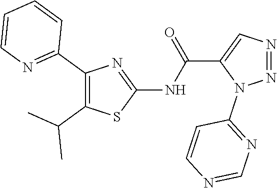 [SMILES: CC(C)c1sc(NC(=O)c2cnnn2-c2ccncn2)nc1-c1ccccn1]

Acid CAS 14 [symbol] 2934-49-5
Amine CAS 1225774-92-4

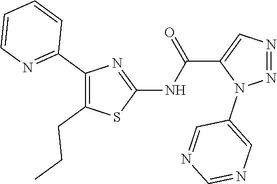 [SMILES: CCCc1sc(NC(=O)c2cnnn2-c2cncnc2)nc1-c1ccccn1]

Acid CAS 1478093-94-5
Amine CAS 2042701-33-5

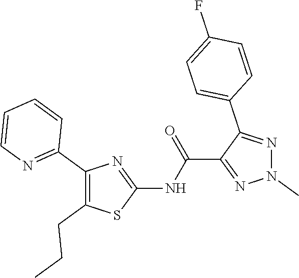 [SMILES: CCCc1sc(NC(=O)c2nn(C)nc2-c2ccc(F)cc2)nc1-c1ccccn1]

Acid CAS 483281-63-6
Amine CAS 2042701-33-5

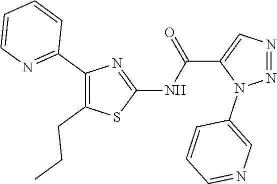 [SMILES: CCCc1sc(NC(=O)c2cnnn2-c2cccnc2)nc1-c1ccccn1]

Acid CAS 1478271-10-1
Amine CAS 2042701-33-5

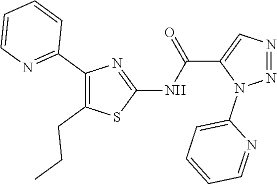 [SMILES: CCCc1sc(NC(=O)c2cnnn2-c2ccccn2)nc1-c1ccccn1]

Acid CAS 1480445-52-0
Amine CAS 2042701-33-5

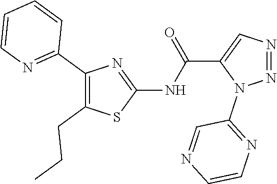 [SMILES: CCCc1sc(NC(=O)c2cnnn2-c2cnccn2)nc1-c1ccccn1]

Acid CAS 2946657-15-2
Amine CAS 2042701-33-5

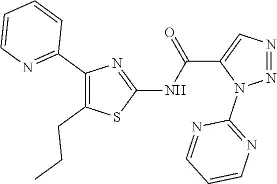 [SMILES: CCCc1sc(NC(=O)c2cnnn2-c2ncccn2)nc1-c1ccccn1]

Acid CAS 2946657-14-1
Amine CAS 2042701-33-5

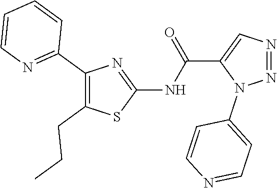 [SMILES: CCCc1sc(NC(=O)c2cnnn2-c2ccncc2)nc1-c1ccccn1]

Acid CAS 1525417-94-0
Amine CAS 2042701-33-5

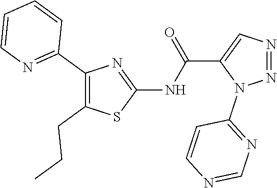 [SMILES: CCCc1sc(NC(=O)c2cnnn2-c2ccncn2)nc1-c1ccccn1]

Acid CAS 1482934-49-5
Amine CAS 2042701-33-5

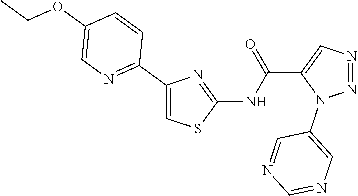 [SMILES: CCOc1ccc(-c2csc(NC(=O)c3cnnn3-c3cncnc3)n2)nc1]

Acid CAS 1478093-94-5
Amine CAS 2768587-25-1

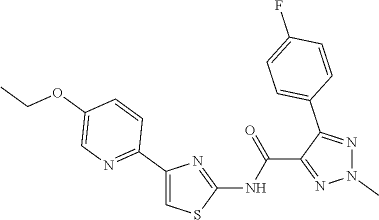 [SMILES: CCOc1ccc(-c2csc(NC(=O)c3nn(C)nc3-c3ccc(F)cc3)n2)nc1]

Acid CAS 483281-63-6
Amine CAS 2768587-25-1

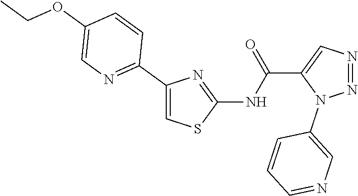 [SMILES: CCOc1ccc(-c2csc(NC(=O)c3cnnn3-c3cccnc3)n2)nc1]

Acid CAS 1478271-10-1
Amine CAS 2768587-25-1

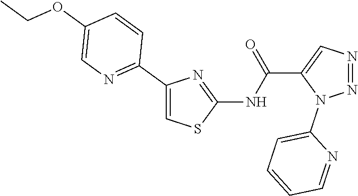 [SMILES: CCOc1ccc(-c2csc(NC(=O)c3cnnn3-c3ccccn3)n2)nc1]

Acid CAS 1480445-52-0
Amine CAS 2768587-25-1

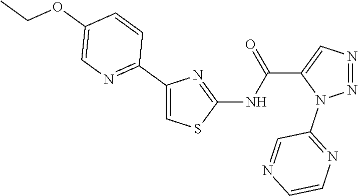 [SMILES: CCOc1ccc(-c2csc(NC(=O)c3cnnn3-c3cnccn3)n2)nc1]

Acid CAS 2946657-15-2
Amine CAS 2768587-25-1

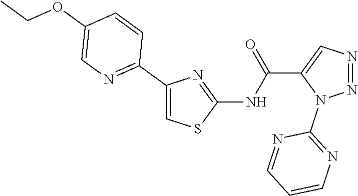 [SMILES: CCOc1ccc(-c2csc(NC(=O)c3cnnn3-c3ncccn3)n2)nc1]

Acid CAS 2946657-14-1
Amine CAS 2768587-25-1

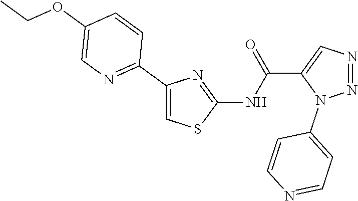 [SMILES: CCOc1ccc(-c2csc(NC(=O)c3cnnn3-c3ccncc3)n2)nc1]

Acid CAS 1525417-94-0
Amine CAS 2768587-25-1

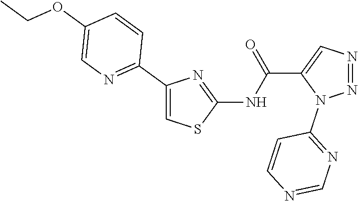 [SMILES: CCOc1ccc(-c2csc(NC(=O)c3cnnn3-c3ccncn3)n2)nc1]

Acid CAS 1482934-49-5
Amine CAS 2768587-25-1

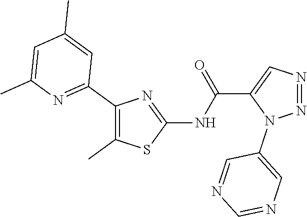 [SMILES: Cc1cc(C)nc(-c2nc(NC(=O)c3cnnn3-c3cncnc3)sc2C)c1]

Acid CAS 1478093-94-5
Amine CAS 2287501-11-3

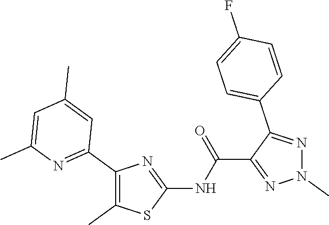

Acid CAS 483281-63-6
Amine CAS 2287501-11-3

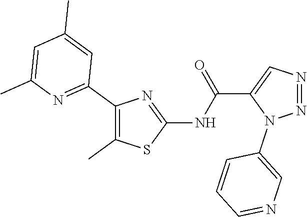 [SMILES: Cc1cc(C)nc(-c2nc(NC(=O)c3cnnn3-c3cccnc3)sc2C)c1]

Acid CAS 1478271-10-1
Amine CAS 2287501-11-3

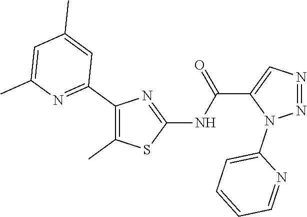 [SMILES: Cc1cc(C)nc(-c2nc(NC(=O)c3cnnn3-c3ccccn3)sc2C)c1]

Acid CAS 1480445-52-0
Amine CAS 2287501-11-3

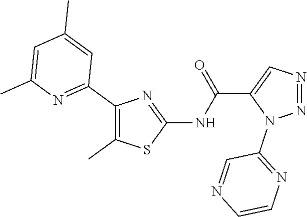 [SMILES: Cc1cc(C)nc(-c2nc(NC(=O)c3cnnn3-c3cnccn3)sc2C)c1]

Acid CAS 2946657-15-2
Amine CAS 2287501-11-3

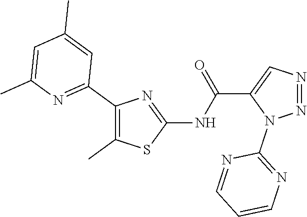 [SMILES: Cc1cc(C)nc(-c2nc(NC(=O)c3cnnn3-c3ncccn3)sc2C)c1]

Acid CAS 2946657-14-1
Amine CAS 2287501-11-3

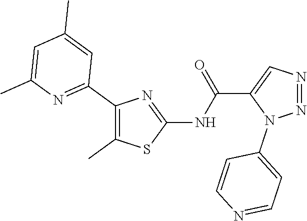 [SMILES: Cc1cc(C)nc(-c2nc(NC(=O)c3cnnn3-c3ccncc3)sc2C)c1]

Acid CAS 1525417-94-0
Amine CAS 2287501-11-3

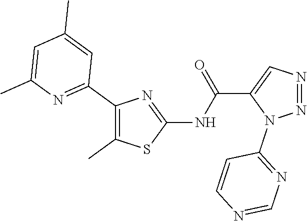 [SMILES: Cc1cc(C)nc(-c2nc(NC(=O)c3cnnn3-c3ccncn3)sc2C)c1]

Acid CAS 1482934-49-5
Amine CAS 2287501-11-3

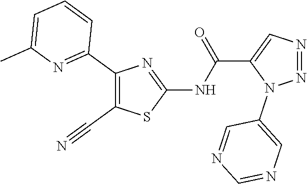 [SMILES: Cc1cccc(-c2nc(NC(=O)c3cnnn3-c3cncnc3)sc2C#N)n1]

Acid CAS 147 [symbol] 093-94-5
Amine CAS 1975266-69-3

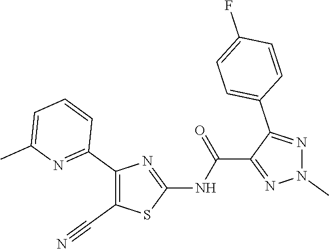

Acid CAS 483281-63-6
Amine CAS 1975266-69-3

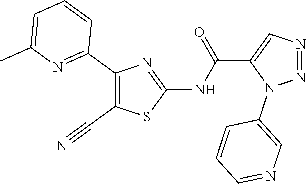 [SMILES: Cc1cccc(-c2nc(NC(=O)c3cnnn3-c3cccnc3)sc2C#N)n1]

Acid CAS 1478271-10-1
Amine CAS 1975266-69-3

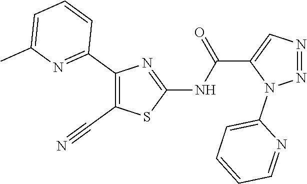 [SMILES: Cc1cccc(-c2nc(NC(=O)c3cnnn3-c3ccccn3)sc2C#N)n1]

Acid CAS 1480445-52-0
Amine CAS 1975266-69-3

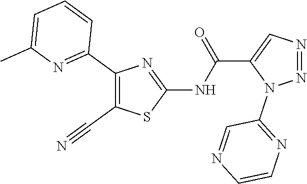 [SMILES: Cc1cccc(-c2nc(NC(=O)c3cnnn3-c3cnccn3)sc2C#N)n1]

Acid CAS 2946657-15-2
Amine CAS 1975266-69-3

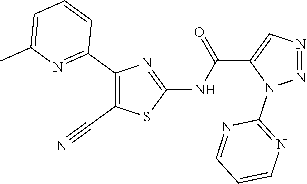 [SMILES: Cc1cccc(-c2nc(NC(=O)c3cnnn3-c3ncccn3)sc2C#N)n1]

Acid CAS 2946657-14-1
Amine CAS 1975266-69-3

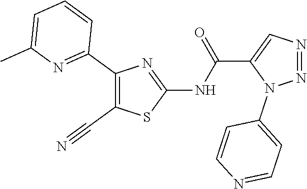 [SMILES: Cc1cccc(-c2nc(NC(=O)c3cnnn3-c3ccncc3)sc2C#N)n1]

Acid CAS 1525417-94-0
Amine CAS 1975266-69-3

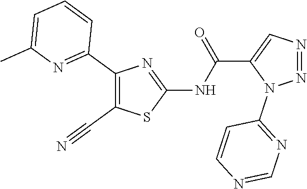 [SMILES: Cc1cccc(-c2nc(NC(=O)c3cnnn3-c3ccncn3)sc2C#N)n1]

Acid CAS 1482934-49-5
Amine CAS 1975266-69-3

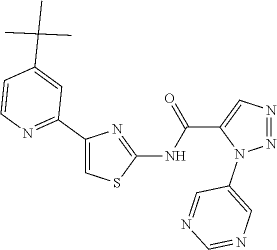 [SMILES: CC(C)(C)c1ccnc(-c2csc(NC(=O)c3cnnn3-c3cncnc3)n2)c1]

Acid CAS 1478093-94-5
Amine CAS 22538668-94-7

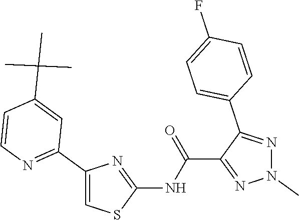 [SMILES: Cn1nc(C(=O)Nc2nc(-c3cc(C(C)(C)C)ccn3)cs2)c(-c2ccc(F)cc2)n1]

Acid CAS 463281-63-6
Amine CAS 2253868-94-7

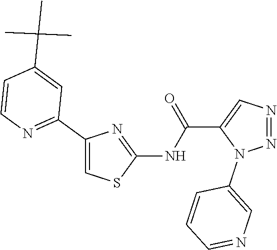 [SMILES: CC(C)(C)c1ccnc(-c2csc(NC(=O)c3cnnn3-c3cccnc3)n2)c1]

Acid CAS 1478271-10-1
Amine CAS 2253868-94-7

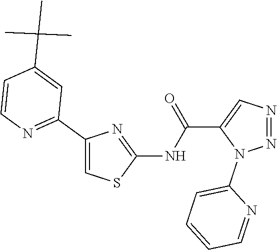 [SMILES: CC(C)(C)c1ccnc(-c2csc(NC(=O)c3cnnn3-c3ccccn3)n2)c1]

Acid CAS 14 [symbol] 0445-52-0
Amine CAS 2253868-94-7

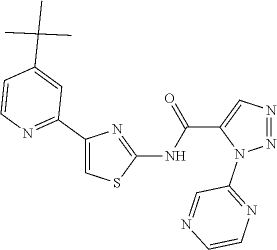 [SMILES: CC(C)(C)c1ccnc(-c2csc(NC(=O)c3cnnn3-c3cnccn3)n2)c1]

Acid CAS 2946657-15-2
Amine CAS 2253868-94-7

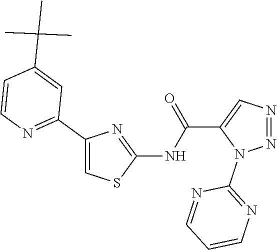 [SMILES: CC(C)(C)c1ccnc(-c2csc(NC(=O)c3cnnn3-c3ncccn3)n2)c1]

Acid CAS 2946657-14-1
Amine CAS 2253868-94-7

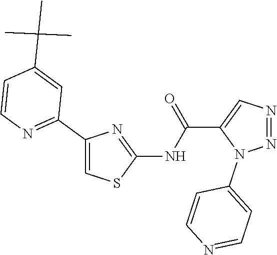 [SMILES: CC(C)(C)c1ccnc(-c2csc(NC(=O)c3cnnn3-c3ccncc3)n2)c1]

Acid CAS 1525417-94-0
Amine CAS 2253868-94-7

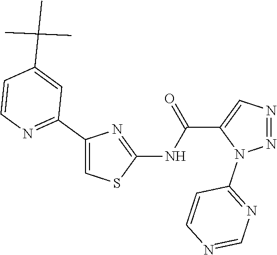 [SMILES: CC(C)(C)c1ccnc(-c2csc(NC(=O)c3cnnn3-c3ccncn3)n2)c1]

Acid CAS 1482934-49-5
Amine CAS 2253868-94-7

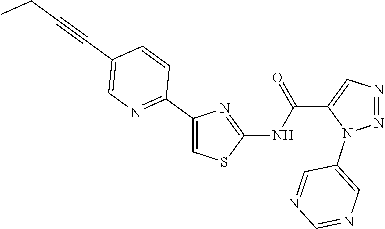 [SMILES: CCC#Cc1ccc(-c2csc(NC(=O)c3cnnn3-c3cncnc3)n2)nc1]

Acid CAS 1478093-94-5
Amine CAS 2538808-37-4

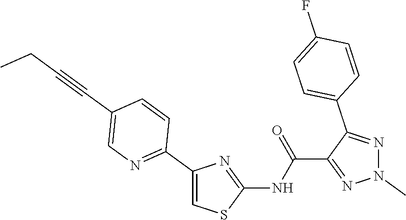 [SMILES: CCC#Cc1ccc(-c2csc(NC(=O)c3nn(C)nc3-c3ccc(F)cc3)n2)nc1]

Acid CAS 4 [symbol] 3281-63-6
Amine CAS 2538808-37-4

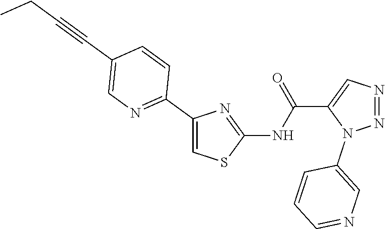 [SMILES: CCC#Cc1ccc(-c2csc(NC(=O)c3cnnn3-c3cccnc3)n2)nc1]

Acid CAS 147 [symbol] 271-10-1
Amine CAS 2538808-37-4

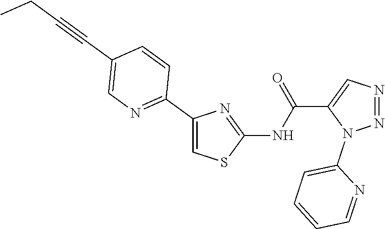 [SMILES: CCC#Cc1ccc(-c2csc(NC(=O)c3cnnn3-c3ccccn3)n2)nc1]

Acid CAS 1480445-52-0
Amine CAS 2538808-37-4

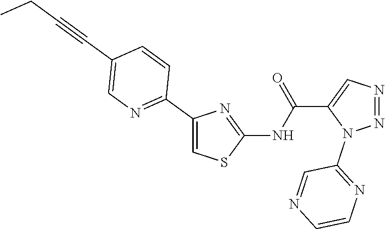 [SMILES: CCC#Cc1ccc(-c2csc(NC(=O)c3cnnn3-c3cnccn3)n2)nc1]

Acid CAS 2946657-15-2
Amine CAS 2538808-37-4

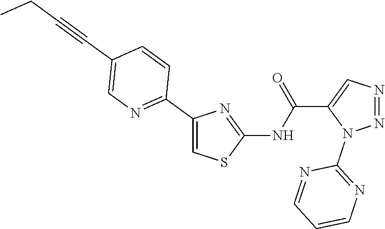 [SMILES: CCC#Cc1ccc(-c2csc(NC(=O)c3cnnn3-c3ncccn3)n2)nc1]

Acid CAS 2946657-14-1
Amine CAS 2538808-37-4

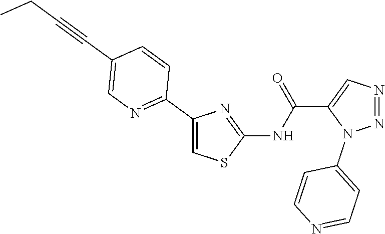 [SMILES: CCC#Cc1ccc(-c2csc(NC(=O)c3cnnn3-c3ccncc3)n2)nc1]

Acid CAS 1525417-94-0
Amine CAS 2538808-37-4

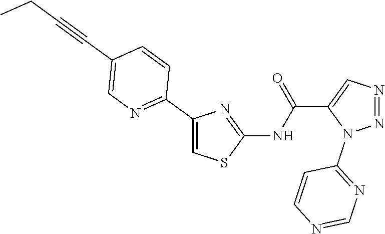 [SMILES: CCC#Cc1ccc(-c2csc(NC(=O)c3cnnn3-c3ccncn3)n2)nc1]

Acid CAS 1482934-49-5
Amine CAS 2538808-37-4

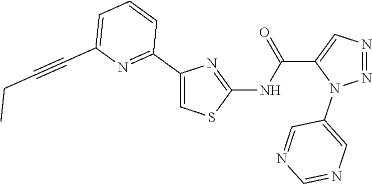 [SMILES: CCC#Cc1cccc(-c2csc(NC(=O)c3cnnn3-c3cncnc3)n2)n1]

Acid CAS 1478093-94-5
Amine CAS 2539204-54-9

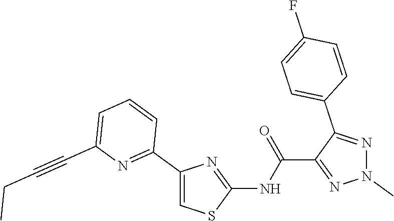 [SMILES: CCC#Cc1cccc(-c2csc(NC(=O)c3nn(C)nc3-c3ccc(F)cc3)n2)n1]

Acid CAS 483281-63-6
Amine CAS 2539204-54-9

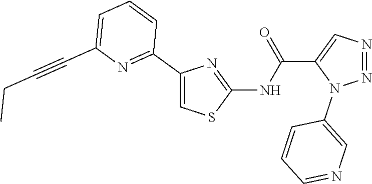 [SMILES: CCC#Cc1cccc(-c2csc(NC(=O)c3cnnn3-c3cccnc3)n2)n1]

Acid CAS 1478271-10-1
Amine CAS 2539204-54-9

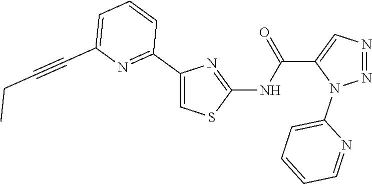 [SMILES: CCC#Cc1cccc(-c2csc(NC(=O)c3cnnn3-c3ccccn3)n2)n1]

Acid CAS 1480445-52-0
Amine CAS 2539204-54-9

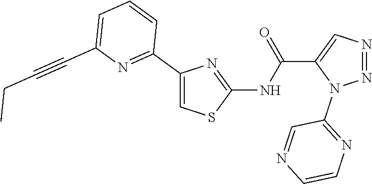 [SMILES: CCC#Cc1cccc(-c2csc(NC(=O)c3cnnn3-c3cnccn3)n2)n1]

Acid CAS 2946657-15-2
Amine CAS 2539204-54-9

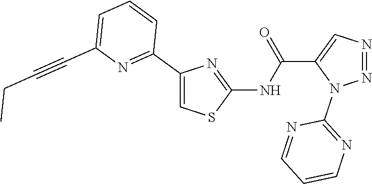 [SMILES: CCC#Cc1cccc(-c2csc(NC(=O)c3cnnn3-c3ncccn3)n2)n1]

Acid CAS 2946657-14-1
Amine CAS 2539204-54-9

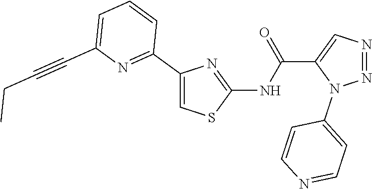 [SMILES: CCC#Cc1cccc(-c2csc(NC(=O)c3cnnn3-c3ccncc3)n2)n1]

Acid CAS 1525417-94-0
Amine CAS 2539204-54-9

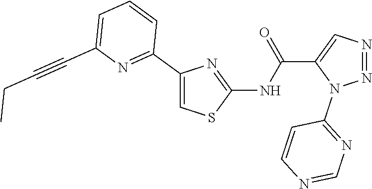 [SMILES: CCC#Cc1cccc(-c2csc(NC(=O)c3cnnn3-c3ccncn3)n2)n1]

Acid CAS 1482934-49-5
Amine CAS 2539204-54-9 [symbol]
indicates data missing or illegible when filed

### Activity in Reporter Assays

All compounds were tested using the human cell line HEK STF293. This cell line carries a Wnt reporter (TCF/LEF1 promoter), which drives expression of the firefly luciferase protein. The level of Wnt activity is directly correlated with the level of luciferase activity (determined using a simple assay). Compounds that inhibit Wnt signaling by reducing luciferase activity in these two cell lines are further tested biochemically. Biochemical confirmation that compounds inhibit Wnt signaling is obtained by immunoblotting for beta-catenin in HEK STF293 cells and demonstrating that its levels are reduced. Compounds were were further tested in a Viability assay using Wnt-dependent HCT116 cell line.

Compounds were prepared as 10 mM stocks for each compound in DMSO. Dilutions were prepared in a 96-well plate in DMSO. The stock dilutions are as follows: 10 mM, 1 mM, 100 μM, 10 μM, 100 nM, and 10 nM. Plates were sealed and stored at −20° C.

HEK STF293 cells were seeded at approximately 25,000-30,000 cells/well in a 96-well (100 μL volume). On the first day, Wnt3a-conditioned media (1:1) were added along with diluted compounds (1:100). For example, for 100 μL of STF293 cells, 100 μL of Wnt3a-conditioned media and 2 μL of drug was added to each well. The final concentrations should therefore be 100 μM, 10 μM, 1 μM, 100 nM, 10 nM, and 1 nM. On the second day, the media was removed and 75 μL of Passive Lysis Buffer (Promega) is added to each well. The plate was shaken at 130 rpm for 15 minutes. For the Steady Glo assay, 45 μL of the lysis was removed and added to a white 96-well plate containing 45 μL/well of Steady Glo solution (Promega). For the Cell Titer assay, 25 μL of the lysis was transferred to a white 96-well plate containing 25 μL/well of Cell Titer solution (Promega). Both Steady Glo and Cell Titer assays were read with a luminescence plate reader. When determining EC50, the Steady Glo values were divided by the Cell Titer values to normalize for cell number.

The control CMV driven cell line assay was performed as recited above for the STF293 assay except that no Wnt3a-conditioned media was added to the plated cells and 1 μL of diluted compound was added instead of 2 μL.

Three concentrations were chosen based on the EC50 curves from the STF293 assay. From the original 10 mM stocks, the following dilutions were prepared in DMSO and stored at −20° C.: 100 μM, 50 μM, and 10 μM.

HEK293 cells were seeded in a 6-well plate at approximately 8.0×10⁵ cells (2 mL per well). On the first day, Wnt3a-conditioned media (1:1) and compounds (1:100) were added to the plated cells. The final concentrations of compounds were 1 μM, 500 nM, and 100 nM. Vehicle (DMSO) and a Wnt3a-conditioned media plus Vehicle samples were also prepared as controls. Lysates were collected (with non-denaturing lysis buffer) after 24 hours incubation, and protein concentrations determined by Bradford Assay. Immunoblotting with an anti-beta-catenin antibody (equivalent amounts of protein/lane for each condition) were subsequently performed to determine beta-catenin levels. HCT116 cells were seeded at 2,500 cells/well in a 96-well dish (Volume: 100 μL/well). On the second day, the 10 mM stock solution of each test compound was thawed at room temperature. Dilutions of the test compound were prepared in DMSO in a V-bottom 96-well plate. The stock compound solution, DMSO-diluted compound solutions, and DMSO were further diluted 500-fold in cell culture media, and 100 μL of the compound- or DMSO-containing cell culture media was added to each well of the cells. The final compound concentrations, therefore, were 10 μM, 3.3.3 μM, 1.11 μM, 370.37 nM, 123.46 nM, 41.15 nM, 13.72 nM, 4.57 nM and zero. The final DMSO concentration was 0.1% in all wells. On the fifth day, 150 μL of media was removed from each well. 50 μL/well Cell Titer Glo reagent (Promega) was added to each well. The plate was shaken at 130 rpm for 15 minutes at room temperature, and the cell viability was determined using a luminescence plate reader.

Results from the HEK STF293 assay disclosed above are shown in Table 3 below. Results from the Viability assay disclosed above are shown in Table 3 below. Potency categories are defined as follows: A: <200 nM, B: 200-500 nM, C: 500-1000 nM, D: 1,000

TABLE 3

| Example number | TOPFlash IC₅₀ category | Viability IC₅₀ category |
|---|---|---|
| 1 | D | D |
| 2 | D | C |
| 3 | D | D |
| 4 | C | D |
| 5 | D | D |
| 6 | B | B |
| 7 | C | C |
| 8 | C | C |
| 9 | C | C |
| 10 | C | D |
| 11 | C | C |
| 12 | B | C |
| 13 | C | C |
| 14 | A | B |
| 15 | C | D |
| 16 | C | C |
| 17 | D | D |
| 18 | B | B |
| 19 | B | B |

### Examples for Formulae (IV), (V), (VI), Ant) (VII)

### Experimental Procedures and Characterization Data

### General Procedure a (Amide Coupling)

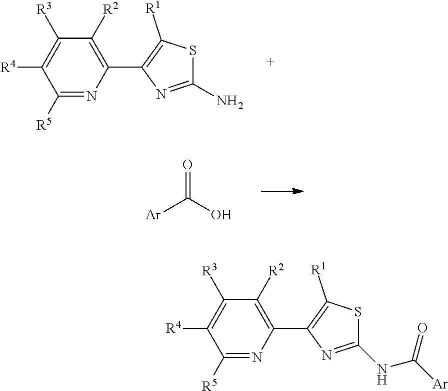

To a stirred solution of an appropriately substituted aromatic carboxylic acid in a suitable organic solvent (e.g., DMF) at room temperature was added 1-2 equivalents of a suitable amide coupling reagent (e.g., HATU) followed by 2-3 equivalents of a suitable organic base (e.g., diisopropylethylamine). A solution of an appropriately substituted aromatic amine in a suitable organic solvent (e.g., DMF) was added to the reaction mixture. The resultant reaction mixture was stirred at room temperature with progress monitored periodically by TLC and/or LCMS. Upon satisfactory conversion of the amine to the desired amide, the reaction mixture was quenched with water and extracted 2-3× with a suitable, water immiscible organic solvent (e.g., EtOAc). The combined organic extracts were washed with aqueous sodium bicarbonate and/or other aqueous media, then dried over a suitable drying agent (e.g., anhydrous MgSO₄). Filtration followed by removal of solvent via rotary evaporation provided a residue that was subsequently purified by normal or reverse phase chromatography to yield the purified amide product.

The following Examples were synthesized using General Procedure A, starting with commercially available or known amine and carboxylic acid building blocks, as shown in Table 4.

TABLE 4

| | | | | Proton NMR | | |
|---|---|---|---|---|---|---|
| | | % | m/z | (400 MHz, | LHS BB | RHS BB |
| Example | Structure | Purity | (predicted) | DMSO-d6) | CAS | CAS |
| 1 | 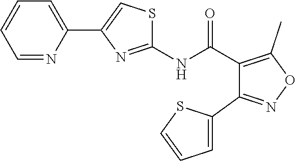 [SMILES: Cc1onc(-c2cccs2)c1C(=O)Nc1nc(-c2ccccn2)cs1] | 99.6 | 369.0 (369.0) | 12.94 (s, 1H), 8.62 (d, J = 4.4 Hz, 1H), 7.95-7.93 (m, 2H), 7.90 (t, J = 8.0 Hz, 1H), 7.78 (d, J = 5.2 Hz, 1H), 7.59 (bs, 1H), 7.36 (t, J = 5.6 Hz, 1H), 7.36 (t, J = 4.4 Hz, 1H), 2.62 (s, 3H) | 30235-26-8 | 83817-53-2 |
| 2 | 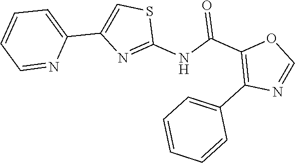 [SMILES: O=C(Nc1nc(-c2ccccn2)cs1)c1ocnc1-c1ccccc1] | >95 | 349.1 (349.1) | 8.78 (s, 1H), 8.62 (ddd, J = 4.8, 1.8, 0.9 Hz, 1H), 8.18- 8.11 (m, 2H), 8.01 (dt, J = 7.9, 1.2 Hz, 1H), 7.95-7.86 (m, 2H), 7.56-7.43 (m, 3H), 7.35 (ddd, J = 7.5, 4.8, 1.3 Hz, 1H), NH not observed | 30235-26-8 | 1240599-64-7 |
| 3 | 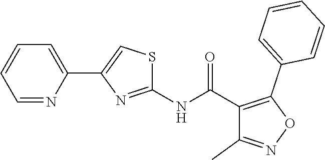 [SMILES: Cc1noc(-c2ccccc2)c1C(=O)Nc1nc(-c2ccccn2)cs1] | 97.4 | 363.0 (363.1) | 12.94 (s, 1H), 8.61 (d, J = 4.4 Hz, 1H), 7.94-7.84 (m, 3H), 7.60-7.50 (m, 2H), 7.58-7.56 (m, 3H), 7.35 (t, J = 5.6 Hz, 1H), 2.40 (s, 3H) | 30235-26-8 | 17153-21-8 |
| 4 | 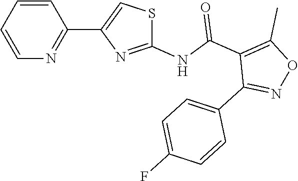 [SMILES: Cc1onc(-c2ccc(F)cc2)c1C(=O)Nc1nc(-c2ccccn2)cs1] | 98.9 | 381.0 (381.1) | 12.83 (s, 1H), 8.61 (d, J = 4.4 Hz, 1H), 7.91-7.85 (m, 3H), 7.71-7.68 (m, 2H), 7.38-7.32 (m, 3H), 2.64 (s, 3H) | 30235-26-8 | 1736-21-6 |
| 5 | 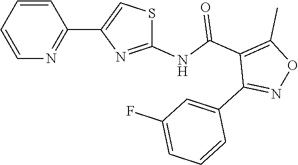 [SMILES: Cc1onc(-c2cccc(F)c2)c1C(=O)Nc1nc(-c2ccccn2)cs1] | 98.7 | 381.1 (381.1) | 12.85 (s, 1H), 8.61 (bs, 1H), 7.93-7.91 (m, 2H), 7.89 (t, J = 7.2 Hz, 1H), 7.59-7.54 (m, 1H), 7.50-7.46 (m, 2H), 7.41-7.32 (m, 2H), 2.65 (s, 3H) | 30235-26-8 | 1736-18-1 |
| 6 | 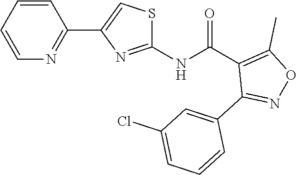 [SMILES: Cc1onc(-c2cccc(Cl)c2)c1C(=O)Nc1nc(-c2ccccn2)cs1] | 98.9 | 397.0 (397.0) | 13.12 (s, 1H), 8.57 (d, J = 4.0 Hz, 1H), 7.93-7.83 (m, 3H), 7.63 (s, 1H), 7.59- 7.49 (m, 3H), 7.34 (t, J = 6.0 Hz, 1H), 2.62 (s, 3H) | 30235-26-8 | 92545-95-4 |
| 7 | 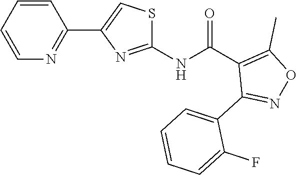 [SMILES: Cc1onc(-c2ccccc2F)c1C(=O)Nc1nc(-c2ccccn2)cs1] | 98.8 | 381.0 (381.1) | 12.73 (s, 1H), 8.61 (bs, 1H), 7.97-7.93 (m, 3H), 7.68-7.61 (m, 2H), 7.41-7.39 (m, 3H), 2.77 (s, 3H) | 30235-26-8 | 1736-20-5 |
| 8 | 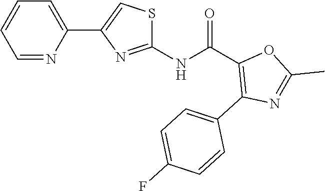 [SMILES: Cc1nc(-c2ccc(F)cc2)c(C(=O)Nc2nc(-c3ccccn3)cs2)o1] | 99.2 | 381.0 (381.1) | 12.77 (s, 1H), 8.63 (d, J = 4.0 Hz, 1H), 8.28-8.24 (m, 2H), 8.02 (d, J = 8.0 Hz, 1H), 7.92-7.89 (m, 2H), 7.38-7.34 (m, 3H), 2.53 (s, 3H) | 30235-26-8 | 914287-73-3 |
| 9 | 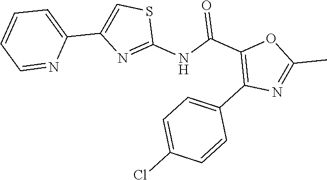 [SMILES: Cc1nc(-c2ccc(Cl)cc2)c(C(=O)Nc2nc(-c3ccccn3)cs2)o1] | 99.3 | 397.1 (397.0) | 12.79 (s, 1H), 8.62 (d, J = 4.4 Hz, 1H), 8.21 (d, J = 8.8 Hz, 2H), 8.01 (d, J = 8.0 Hz, 1H), 7.92- 7.88 (m, 2H), 7.56 (d, J = 8.4 Hz, 2H), 7.36-7.33 (m, 1H), 2.59 (s, 3H) | 30235-26-8 | 1368745-66-7 |
| 10 | 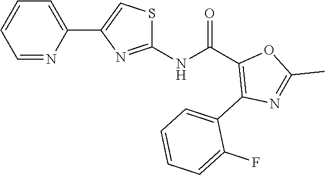 [SMILES: Cc1nc(-c2ccccc2F)c(C(=O)Nc2nc(-c3ccccn3)cs2)o1] | 99.1 | 380.9 (381.1) | 12.88 (s, 1H), 8.61 (bs, 1H), 7.99 (d, J = 4.8 Hz, 1H), 8.92-8.89 (m, 2H), 7.68 (t, J = 8.0 Hz, 1H), 7.54-7.52 (m, 1H), 7.35-7.30 (m, 3H), 2.90 (s, 3H) | 30235-26-8 | 1368655-22-4 |
| 11 | 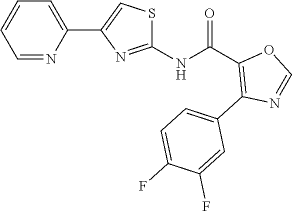 [SMILES: O=C(Nc1nc(-c2ccccn2)cs1)c1ocnc1-c1ccc(F)c(F)c1] | >95 | 385.0 (385.0) | 8.83 (s, 1H), 8.62 (ddd, J = 4.8, 1.8, 0.9 Hz, 1H), 8.29 (ddd, J = 12.4, 8.0, 2.2 Hz, 1H), 8.11- 8.04 (m, 1H), 8.01 (dt, J = 7.9, 1.1 Hz, 1H), 7.94 (s, 1H), 7.90 (td, J = 7.7, 1.8 Hz, 1H), 7.61 (dt, J = 10.7, 8.6 Hz, 1H), 7.35 (ddd, J = 7.5, 4.8, 1.3 Hz, | 30235-26-8 | 1368424-75-2 |
| | | | | 1H), NH not | | |
| | | | | observed | | |
| 12 | 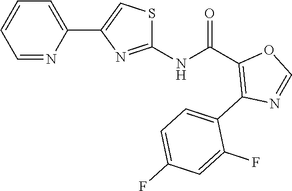 [SMILES: O=C(Nc1nc(-c2ccccn2)cs1)c1ocnc1-c1ccc(F)cc1F] | >95 | 385.0 (385.0) | 8.63-8.60 (m, 1H), 8.02-7.72 (m, 5H), 7.45-7.38 (m, 1H), 7.37-7.32 (m, 1H), 7.29-7.23 (m, 1H), NH not observed | 30235-26-8 | 1368645-00-4 |
| 13 | 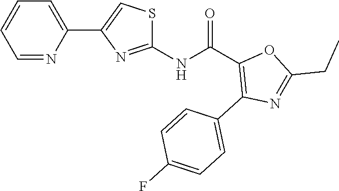 [SMILES: CCc1nc(-c2ccc(F)cc2)c(C(=O)Nc2nc(-c3ccccn3)cs2)o1] | 97.4 | 395.0 (395.1) | 12.88 (s, 1H), 8.62- 8.61 (m, 1H), 8.28- 8.24 (m, 2H), 8.04 (d, J = 8.0 Hz, 1H), 7.92-7.88 (m, 2H), 7.37-7.32 (m, 3H), 2.95 (q, J = 8.0 Hz, 2H), 1.41 (t, J = 7.6 Hz, 3H) | 30235-26-8 | 1489246-42-5 |
| 14 | 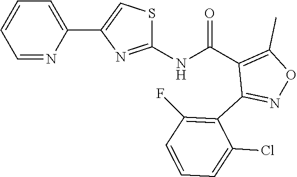 [SMILES: Cc1onc(-c2c(F)cccc2Cl)c1C(=O)Nc1nc(-c2ccccn2)cs1] | 99.1 | 415.0 (415.0) | 12.76 (s, 1H), 8.61 (d, J = 4.4 Hz, 1H), 7.95 (t, J = 7.6 Hz, 1H), 7.90-7.86 (m, 2H), 7.65-7.59 (m, 1H), 7.51 (d, J = 7.6 Hz, 1H), 7.45 (t, J = 8.8 Hz, 1H), 7.35 (t, J = 6.0 Hz, 1H), 2.76 (s, 3H) | 30235-26-8 | 3919-74-2 |
| 15 | 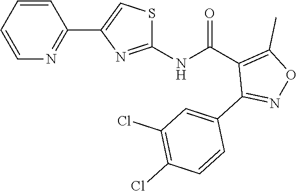 [SMILES: Cc1onc(-c2ccc(Cl)c(Cl)c2)c1C(=O)Nc1nc(-c2ccccn2)cs1] | 99.8 | 430.9 (431.0) | 12.85 (s, 1H), 8.61 (bs, 1H), 8.93-8.85 (m, 4H), 7.81 (d, J = 8.4 Hz, 1H), 7.63 (d, J = 8.4 Hz, 1H), 7.35 (t, J = 6.0 Hz, 1H), 2.67 (s, 3H) | 30235-26-8 | 1778948-18-7 |
| 16 | 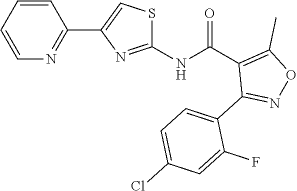 [SMILES: Cc1onc(-c2ccc(Cl)cc2F)c1C(=O)Nc1nc(-c2ccccn2)cs1] | 99.6 | 414.9 (415.0) | 12.67 (s, 1H), 8.61 (bs, 1H), 7.93 (d, J = 7.6 Hz, 1H), 7.88-7.85 (m, 2H), 7.77-7.63 (m, 2H), 7.51 (d, J = 7.6 Hz, 1H), 7.35 (t, J = 7.6 Hz, 1H), 2.64 (s, 3H) | 30235-26-8 | 1778948-18-7 |
| 17 | 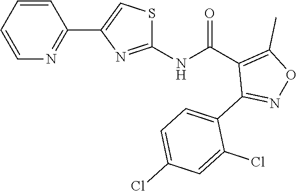 [SMILES: Cc1onc(-c2ccc(Cl)cc2Cl)c1C(=O)Nc1nc(-c2ccccn2)cs1] | 99.7 | 430.8 (431.0) | 12.88 (s, 1H), 8.61 (bs, 1H), 7.93-7.85 (m, 2H), 7.71-7.68 (m, 2H), 7.38-7.32 (m, 3H), 2.64 (s, 3H) | 30235-26-8 | 4402-78-2 |
| 18 | 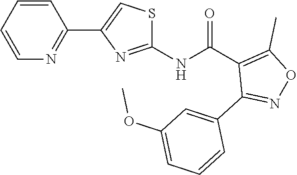 [SMILES: COc1cccc(-c2noc(C)c2C(=O)Nc2nc(-c3ccccn3)cs2)c1] | 98.3 | 393.1 (393.1) | 12.83 (s, 1H), 8.61(bs, 1H), 7.92- 7.91 (m, 2H), 7.89 (t, J = 7.2 Hz, 1H), 7.44 (t, J = 7.6 Hz, 1H), 7.21-7.18 (m, 3H), 7.11-7.08 (m, 1H), 3.75 (s, 3H), 2.63 (s, 3H) | 30235-26-8 | 93002-09-6 |
| 19 | 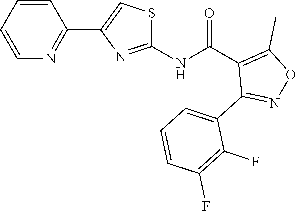 [SMILES: Cc1onc(-c2cccc(F)c2F)c1C(=O)Nc1nc(-c2ccccn2)cs1] | 99.9 | 399.0 (399.1) | 12.89 (s, 1H), 8.61 (bs, 1H), 7.94-7.89 (m, 3H), 7.68-7.64 (m, 1H), 7.49 7.33 (m, 3H), 2.70 (s, 3H) | 30235-26-8 | 1494042-42-0 |
| 20 | 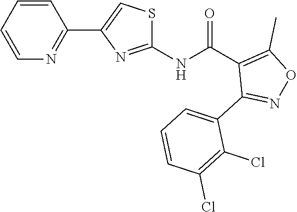 [SMILES: Cc1onc(-c2cccc(Cl)c2Cl)c1C(=O)Nc1nc(-c2ccccn2)cs1] | 99.4 | 431.0 (431.0) | 12.75 (s, 1H), 8.61 (bs, 1H), 7.94-7.83 (m, 4H), 7.56-7.53 (m, 2H), 7.35 (t, J = 6.0 Hz, 1H), 2.73 (s, 3H) | 30235-26-8 | 4402-75-9 |
| 21 | 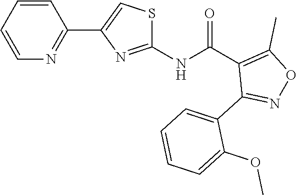 [SMILES: COc1ccccc1-c1noc(C)c1C(=O)Nc1nc(-c2ccccn2)cs1] | 95.1 | 393.1 (393.1) | 12.56 (s, 1H), 8.60 (d, J = 4.4 Hz, 1H), 7.93-7.84 (m, 3H), 7.56 (d, J = 7.2 Hz, 1H), 7.50 (t, J = 8.0 Hz, 1H), 7.33 (t, J = 6.0 Hz, 1H), 7.13-7.07 (m, 2H), 3.50 (s, 3H), 2.62 (s, 3H) | 30235-26-8 | 93041-44-2 |
| 22 | 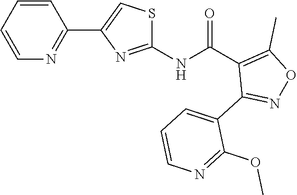 [SMILES: COc1ncccc1-c1noc(C)c1C(=O)Nc1nc(-c2ccccn2)cs1] | 99.5 | 394.2 (394.1) | 12.67 (s, 1H), 8.61 (bs, 1H), 8.33 (t, J = 7.6 Hz, 1H), 8.00 (d, J = 7.6 Hz, 1H), 7.93-7.84 (m, 3H), 7.35 (t, J = 7.6 Hz, 1H), 7.20 (t, J = 8.4 Hz, 1H), 3.61 (s, 3H), 2.64 (s, 3H) | 30235-26-8 | 1370418-93-1 |
| 23 | 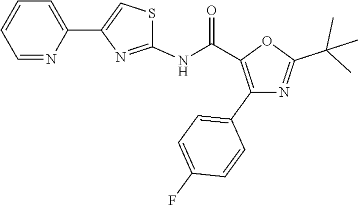 [SMILES: CC(C)(C)c1nc(-c2ccc(F)cc2)c(C(=O)Nc2nc(-c3ccccn3)cs2)o1] | 99.3 | 423.1 (423.1) | 12.79 (s, 1H), 8.56 (d, J = 4.4 Hz, 1H), 8.16-8.12 (m, 2H), 8.05 (d, J = 7.6 Hz, 1H), 7.91-7.86 (m, 2H), 7.36-7.27 (m, 3H), 1.43 (s, 9H) | 30235-26-8 | 936128-27-7 |
| 24 | 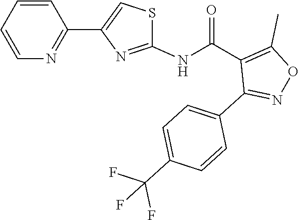 [SMILES: Cc1onc(-c2ccc(C(F)(F)F)cc2)c1C(=O)Nc1nc(-c2ccccn2)cs1] | 99.4 | 431.0 (431.1) | 12.88 (s, 1H), 8.61 (d, J = 4.4 Hz, 1H), 7.92-7.84 (m, 7H), 7.35 (t, J = 5.6 Hz, 1H), 2.67 (s, 3H) | 30235-26-8 | 943130-82-3 |
| 25 | 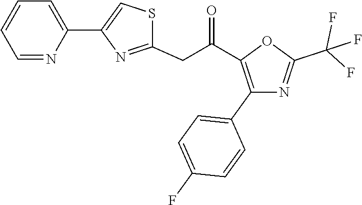 [SMILES: O=C(Cc1nc(-c2ccccn2)cs1)c1oc(C(F)(F)F)nc1-c1ccc(F)cc1] | 99.3 | 435.4 (435.0) | 13.40 (bs, 1H), 8.63 (d, J = 4.0 Hz, 1H), 8.25-8.19 (m, 2H), 8.03 (d, J = 7.6 Hz, 1H), 7.97 (s, 1H), 7.92 (d, J = 8.0 Hz, 1H) 7.44- 7.34 (m, 3H) | 30235-26-8 | 1422175-36-7 |
| 26 | 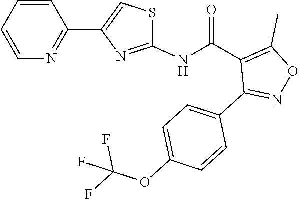 [SMILES: Cc1onc(-c2ccc(OC(F)(F)F)cc2)c1C(=O)Nc1nc(-c2ccccn2)cs1] | 99.5 | 447.0 (447.1) | 12.85 (s, 1H), 8.60 (bs, 1H), 7.91-7.85 (m, 3H), 7.79 (d, J = 7.6 Hz, 2H), 7.54 (d, J = 7.6 Hz, 2H), 7.34 (t, J = 8.4 Hz, 1H), 2.66 (s, 3H) | 30235-26-8 | 1402881-82-6 |
| 27 | 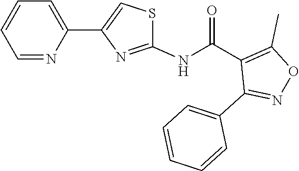 [SMILES: Cc1onc(-c2ccccc2)c1C(=O)Nc1nc(-c2ccccn2)cs1] | 99.1 | 363.0 (363.1) | 12.85 (s, 1H), 8.61 (bs, 1H), 7.92-7.84 (m, 3H), 7.64-7.62 (m, 2H), 7.52-7.50 (m, 3H), 7.35 (t, J = 5.2 Hz, 1H), 2.63 (s, 3H) | 30235-26-8 | 1136-45-4 |
| 28 | 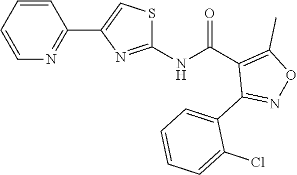 [SMILES: Cc1onc(-c2ccccc2Cl)c1C(=O)Nc1nc(-c2ccccn2)cs1] | 98.5 | 397.0 (397.0) | 12.67 (s, 1H), 8.60 (d, J = 4.4 Hz, 1H), 7.93-7.86 (m, 3H), 7.60-7.50 (m, 4H), 7.35 (t, J = 6.0 Hz, 1H), 2.71 (s, 3H) | 30235-26-8 | 23598-72-3 |
| 29 | 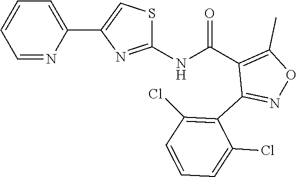 [SMILES: Cc1onc(-c2c(Cl)cccc2Cl)c1C(=O)Nc1nc(-c2ccccn2)cs1] | 99.2 | 431.0 (431.0) | 12.72 (s, 1H), 8.61 (bs, 1H), 7.97 (d, J = 8 Hz, 1H), 7.90- 7.85 (m, 2H), 7.65- 7.55 (m, 3H), 7.35 (t, J = 6.4 Hz, 1H), 2.79 (s, 3H) | 30235-26-8 | 3919-76-4 |

### Synthesis of Example 30

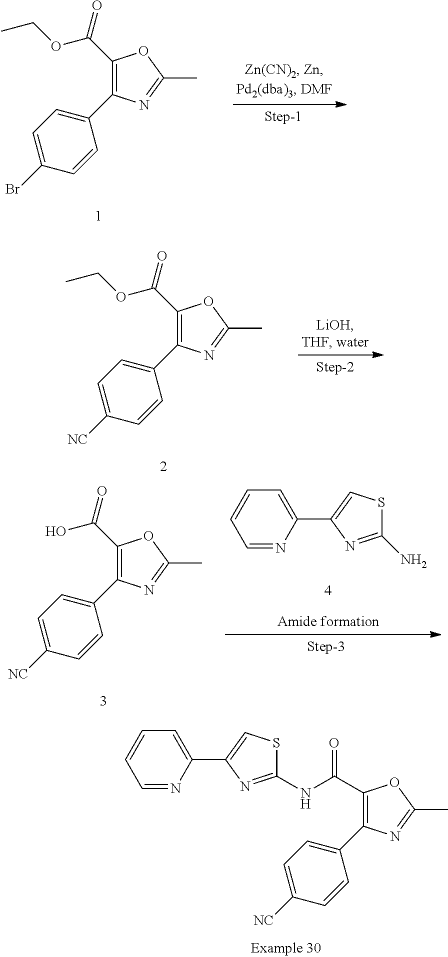 [SMILES: CCOC(=O)c1oc(C)nc1-c1ccc(Br)cc1 ; CCOC(=O)c1oc(C)nc1-c1ccc(C#N)cc1 ; Cc1nc(-c2ccc(C#N)cc2)c(C(=O)O)o1 ; Nc1nc(-c2ccccn2)cs1 ; Cc1nc(-c2ccc(C#N)cc2)c(C(=O)Nc2nc(-c3ccccn3)cs2)o1]

### Step-1: Ethyl 4-(4-cyanophenyl)-2-methyloxazole-5-carboxylate (2)

To a stirred mixture of ethyl 4-(4-bromophenyl)-2-methyloxazole-5-carboxylate (1) (CAS #1423705-99-0) (1 g, 3.32 mmol, 1 eq) in DMF (10 mL), Zn(CN)₂ (226 mg, 1.93 mol, 1.1 eq), Zn (54 mg, 0.83 mol, 0.2 eq), Pd₂(dba)₃ (60 mg, 0.06 mmol, 0.02 eq) and (PPh₃)₂ferrocene (70 mg, 0.12 mmol, 0.04 eq) were and the reaction mixture was degassed for 15 min. The mixture was stirred at 120° C. for 16 h. The progress of the reaction was monitored by TLC (M.Ph: 10% EtOAc in n-hexane). After completion of reaction, the reaction mixture was diluted with water. The aqueous layer was extracted with EtOAc (2×150 mL). The organic layer was washed with brine, dried over anhydrous Na₂SO₄ and concentrated under reduced pressure. The crude compound was purified by flash column chromatography (0-10% EtOAc in n-hexane) to afford 2 (780 mg, 94%) as a viscous mass. ¹H NMR (400 MHz, DMSO-d₆) δ 8.23 (d, J=6.8 Hz, 2H), 7.94 (d, J=7.2 Hz, 2H), 2.45 (q, J=6.8 Hz, 2H); LC-MS: m/z 257.0 [M+H]⁺.

### Step-2: 4-(4-Cyanophenyl)-2-methyloxazole-5-carboxylic acid (3)

To a stirred mixture of ethyl 4-(4-cyanophenyl)-2-methyloxazole-5-carboxylate (2) (150 mg, 0.585 mmol, 1 eq) in THF:H₂O (3:1, 4 mL), LiOH·H₂O (49.2 mg, 1.17 mmol, 2 eq) was added at RT. The reaction mixture was stirred at RT for 3 h. The progress of the reaction was monitored by TLC (M.Ph: 50% EtOAc in n-hexane). After completion of reaction, the reaction mixture was concentrated under reduced pressure. The crude residue was diluted with water and pH was adjusted to ˜3 by adding 1N HCl solution. The precipitated solid was filtered, washed with water and dried under reduced pressure to afford compound 3 (92 mg, 69%) as an off-white solid. ¹H NMR (400 MHz, DMSO-d₆) δ 8.19 (d, J=8.8 Hz, 2H), 7.96 (d, J=8.4 Hz, 2H), 4.35 (q, J=6.8 Hz, 2H), 3.31 (s, 3H), 1.30 (t, J=6.8 Hz, 3H); LC-MS: m/z 229.05 [M+H]⁺.

### Step-3: Example 30

In line with the General Procedure A, 4-(4-cyanophenyl)-2-methyloxazole-5-carboxylic acid (3) (90 mg, 0.396 mmol, 1 eq) was coupled with 4-(pyridin-2-yl)thiazol-2-amine (4) (70 mg, 0.396 mmol, 1.0 eq) to afford Example 30 (4 mg, 2.6%) as an off-white. ¹H NMR (400 MHz, DMSO-d₆) δ 12.95 (bs, 1H), 8.63 (bs, 1H), 8.36 (d, J=8.4 Hz, 2H), 8.03-7.92 (m, 5H), 7.38-7.35 (m, 1H), 2.61 (s, 3H); LC-MS: m/z 388.0 [M+H]⁺; HPLC: 98.2%.

### Synthesis of Example 31

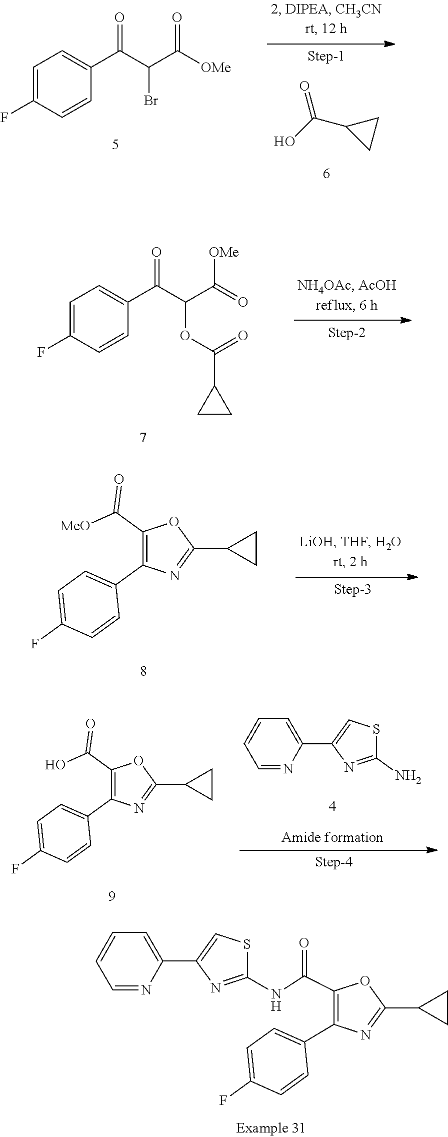

### Example 31

### Step-1: 1-(4-fluorophenyl)-3-methoxy-1,3-dioxopropan-2-yl cyclopropane carboxylate (7)

A solution of methyl 2-bromo-3-(4-fluorophenyl)-3-oxopropanoate (5) (CAS #1001922-15-1) (400 mg, 1.45 mmol, 1 eq), cyclopropanecarboxylic acid (6) (250 mg, 2.91 mmol, 2 eq) and DIPEA (0.5 mL, 2.91 mmol, 2 eq) in CH₃CN (6 mL) was stirred at rt for 12 h. Progress of reaction was monitored by TLC. After completion, the reaction mixture was diluted with EtOAc (25 mL) and organic layer was washed with H₂O (15 mL) and brine solution (15 mL). The crude obtained was purified by column chromatography (silica 100-200 mesh; 22% EtOAc in Hexanes) to afford 7 (305 mg, 75%). ¹H NMR (400 MHz, CDCl₃) δ 8.11-8.02 (m, 2H), 7.19 (t, J=8.5 Hz, 2H), 6.31 (s, 1H), 3.82 (s, 3H), 1.88-1.77 (m, 1H), 1.15-1.08 (m, 2H), 1.03-0.96 (m, 2H).

### Step-2: methyl 2-cyclopropyl-4-(4-fluorophenyl)oxazole-5-carboxylate (8)

A solution of 1-(4-fluorophenyl)-3-methoxy-1,3-dioxopropan-2-yl cyclopropanecarboxylate (7) (300 mg, 1.07 mmol, 1 eq), NH₄OAc (165 mg, 2.14 mmol, 2 eq) in AcOH (4 mL) was heated at reflux for 6 h. Progress of reaction was monitored by TLC. After completion, the reaction mixture was diluted with EtOAc (30 mL) and organic layer was washed with H₂O (3×10 mL) and brine solution (2×10 mL). The crude obtained was purified by column chromatography (silica 100-200 mesh; 30% EtOAc in Hexanes) to afford (8) (258 mg, 92%). ¹H NMR (400 MHz, DMSO-d₆) δ 8.10-8.01 (m, 2H), 7.36-7.24 (m, 2H), 3.82 (s, 3H), 2.31-2.17 (m, 1H), 1.20-1.14 (m, 2H), 1.12-1.06 (m, 2H); LC-MS: m/z 262.0 [M+H]⁺.

### Step-3: 2-cyclopropyl-4-(4-fluorophenyl)oxazole-5-carboxylic acid (9)

To a solution of methyl 2-cyclopropyl-4-(4-fluorophenyl)oxazole-5-carboxylate (8) (250 mg, 0.95 mmol, 1 eq) in THF:H₂O (4:1; 4 mL) was added LiGH (46 mg, 1.91 mmol, 2 eq) and the reaction mixture was stirred at rt for 2 h. Progress of reaction was monitored by TLC.

After completion, the reaction mixture was concentrated, and crude was washed with Et₂O (5 mL) and acidified with 2N HCl. The solid obtained was filtered and washed with Pentane (5 mL) to afford (9) (208 mg, crude). This was used as such for the next reaction. ¹H NMR (400 MHz, DMSO-d₆) δ =13.53 (d, J=2.0 Hz, 1H), 8.20-7.97 (m, 2H), 7.29 (t, J=8.7 Hz, 2H), 2.29-2.14 (m, 1H), 1.18-1.12 (m, 2H), 1.10-1.04 (m, 2H).

### Step-4: Example 31

In line with General Procedure A, compound 9 was coupled with 4-(pyridin-2-yl)thiazol-2-amine (4) to afford Example 31 as an off-white solid. ¹H NMR (400 MHz, DMSO-d₆) δ 12.98 (s, 1H), 8.56 (d, J=4.4 Hz, 1H), 8.16-8.12 (m, 2H), 8.04 (d, J=8.0 Hz, 1H), 7.90-7.84 (m, 2H), 7.35-7.26 (m, 3H), 2.26-2.22 (m, 1H), 1.29-1.24 (m, 2H), 1.19-1.16 (m, 2H); LC-MS: m/z 407.1 [M+H]⁺; HPLC: 99.2%.

### Synthesis of Example 32

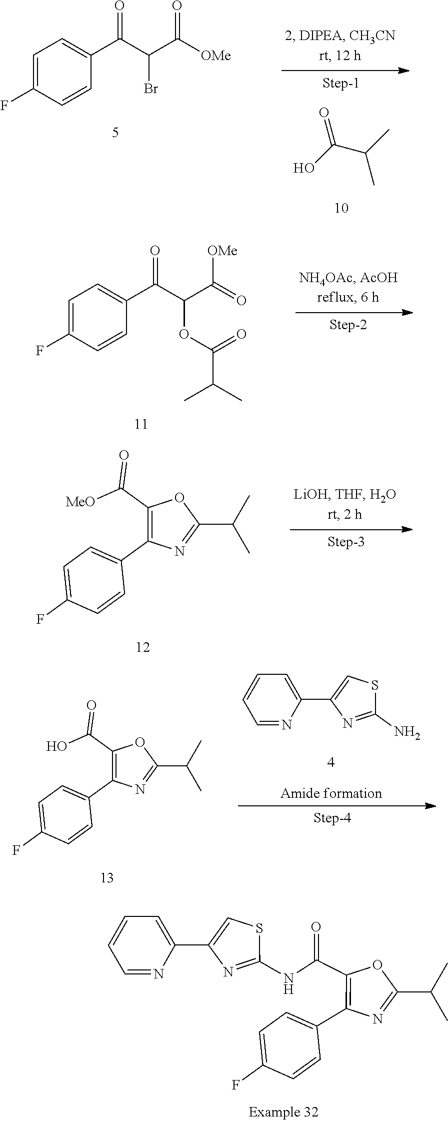

### Step-1: methyl 3-(4-fluorophenyl)-2-(isobutyryloxy)-3-oxopropanoate (11)

A solution of methyl 2-bromo-3-(4-fluorophenyl)-3-oxopropanoate (5) (300 mg, 1.09 mmol, 1 eq), isobutyric acid (10) (192 mg, 2.18 mmol, 2 eq) and DIPEA (0.4 mL, 2.18 mmol, 2 eq) in CH₃CN (4 mL) was stirred at rt for 12 h. Progress of reaction was monitored by TLC.

After completion, the reaction mixture was diluted with EtOAc (20 mL) and organic layer was washed with H₂O (10 mL) and brine solution (10 mL). The crude obtained was purified by column chromatography (silica 100-200 mesh; 20% EtOAc in Hexanes) to afford (11) (230 mg, 74%). ¹H NMR (400 MHz, CHLOROFORM-d) 6=8.05 (dd, J=5.9, 7.8 Hz, 2H), 7.18 (t, J=8.6 Hz, 2H), 6.27 (s, 1H), 3.81 (s, 3H), 2.82-2.70 (m, 1H), 1.24 (dd, J=7.1, 13.0 Hz, 6H); LC-MS: m/z 282.9 [M+H]⁺.

### Step-2: methyl 4-(4-fluorophenyl)-2-isopropyloxazole-5-carboxylate (12)

A solution of methyl 3-(4-fluorophenyl)-2-(isobutyryloxy)-3-oxopropanoate (11) (330 mg, 1.17 mmol, 1 eq), NH₄OAc (182 mg, 2.34 mmol, 2 eq) in AcOH (4 mL) was heated at reflux for 6 h. Progress of reaction was monitored by TLC. After completion, the reaction mixture was diluted with EtOAc (30 mL) and organic layer was washed with H₂O (3×10 mL) and brine solution (2×10 mL). The crude obtained was purified by column chromatography (silica 100-200 mesh; 30% EtOAc in Hexanes) to afford (12) (300 mg, 97%). LC-MS: m/z 264 [M+H]⁺.

### Step-3: 4-(4-fluorophenyl)-2-isopropyloxazole-5-carboxylic acid (13)

To a solution of methyl 4-(4-fluorophenyl)-2-isopropyloxazole-5-carboxylate (12) (295 mg, 1.12 mmol, 1 eq) in THF:H₂O (4:1; 6 mL) was added LiGH (54 mg, 2.24 mmol, 2 eq) and reaction mixture was stirred at rt for 2 h. Progress of reaction was monitored by TLC. After completion, the reaction mixture was concentrated, and crude was washed with Et₂O (5 mL) and acidified with 2N HCl. The solid obtained was filtered and washed with Pentane (5 mL) to afford (12) (250 mg, crude). This was used as such for the next reaction. ¹H NMR (400 MHz, DMSO-d₆) δ=13.68-13.40 (m, 1H), 8.10 (dd, J=5.7, 8.7 Hz, 2H), 7.30 (t, J=9.0 Hz, 2H), 3.25-3.11 (m, 1H), 1.33 (d, J=7.0 Hz, 6H); LC-MS: m/z 250.0 [M+H]⁺.

### Step-4: Example 32

In line with General Procedure A, compound 13 was coupled with 4-(pyridin-2-yl)thiazol-2-amine (4) to afford Example 32 as an off-white solid. ¹H NMR (400 MHz, DMSO-d₆) δ 13.39 (s, 1H), 8.56 (d, J=3.6 Hz, 1H), 8.17-8.14 (m, 2H), 8.05 (d, J=7.2 Hz, 1H), 7.91-7.86 (m, 2H), 7.36-7.28 (m, 3H), 3.24-3.14 (m, 1H), 1.39-1.37 (m, 6H); LC-MS: m/z 409.1 [M+H]⁺; HPLC: 99.2%.

### Synthesis of Example 33

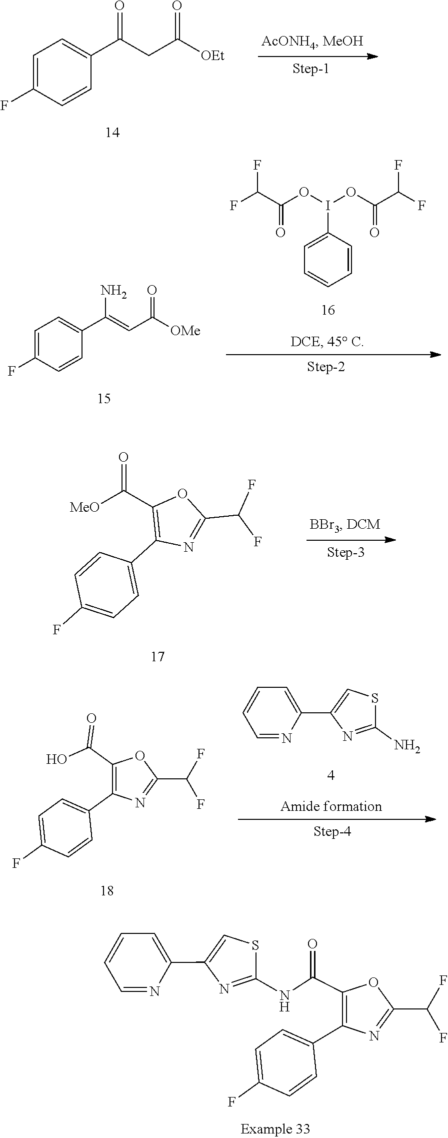

### Step-1: Ethyl(Z)-3-amino-3-(4-fluorophenyl)acrylate (15)

To a stirred solution of ethyl 3-(4-fluorophenyl)-3-oxopropanoate (14) (5 g, 25.4 mmol, 1 eq) in MeOH (50 mL, 10 vol.), NH₄OAc (9.8 g, 127.4 mmol, 5 eq) was added and reaction mixture was refluxed for 16 h. The progress of the reaction was monitored by TLC. After completion of reaction, the mixture was concentrated to dryness and purified through silica gel column chromatography to afford 15 (2 g, 60%) as an off-white solid. ¹H NMR (400 MHz, DMSO-d₆) δ 8.13 (bs, 1H), 7.68 (dd, J=5.6 Hz, 2.8 Hz, 2H), 7.30-7.26 (m, 2H), 4.77 (s, 1H), 3.57 (s, 3H); LC-MS: m/z 196.6 [M+H]⁺.

### Step-2: Methyl 2-(difluoromethyl)-4-(4-fluorophenyl)oxazole-5-carboxylate (17)

To a stirred solution of ethyl(Z)-3-amino-3-(4-fluorophenyl)acrylate (15) (3 g, 15.36 mmol, 1 eq) in 1,2-dichloroethane (50 mL), (2,2-Difluoroacetato-κO)phenyliodine (16) (7.2 g, 18.44 mmol, 1.2 eq) was added and reaction mixture was stirred at 45° C. for 16 h. The progress of the reaction was monitored by TLC. After completion of reaction, the mixture was concentrated to dryness and purified through silica gel column chromatography to afford 17 (3.10 g, 61%) as an oily mass. ¹H NMR (400 MHz, DMSO-d₆) δ 8.10-8.06 (m, 2H), 7.48-7.23 (m, 3H), 3.87 (s, 3H); LC-MS: m/z 257.78 [M+H]⁺.

### Step-3: 2-(Difluoromethyl)-4-(4-fluorophenyl)oxazole-5-carboxylic acid (18)

To a stirred solution of methyl 2-(difluoromethyl)-4-(4-fluorophenyl)oxazole-5-carboxylate (4) (1.1 g, 4.05 mmol, 1 eq) in DCM (20 mL), BBr₃ (1M in DCM, 10.1 mL, 40.5 mmol, 10 eq) was added at 0° C. and reaction mixture was stirred at RT for 16 h. The progress of the reaction was monitored by TLC. After completion of reaction, the mixture was quenched with MeOH (5 mL) at 0° C. The resulting mixture was concentrated to dryness and the crude was diluted with ice water. The aqueous layer was extracted with EtOAc (2×75 mL). The organic layer was washed thoroughly with cold water and brine, dried over anhydrous Na₂SO₄and concentrated under reduced pressure to afford 18 (700 mg, 70%) as brown solid. ¹H NMR (400 MHz, DMSO-d₆) δ 14.13 (s, 1H), 8.12-8.09 (m, 2H), 7.48-7.22 (m, 3H); LC-MS: m/z 257.78 [M+H]⁺.

### Step-4: Example 33

To a stirred solution of 2-(difluoromethyl)-4-(4-fluorophenyl)oxazole-5-carboxylic acid (18) (0.7 g, 2.7 mmol, 1 eq) in DMF (5 mL), DIPEA (0.52 g, 4.08 mmol, 1.5 eq) and HATU (1.5 g, 4.08 mmol, 1.5 eq) were added and the reaction mixture was stirred for 5 min. To the resulting reaction mixture, 4-(pyridin-2-yl)thiazol-2-amine (4) (0.53 g, 2.9 mmol, 1.1 eq) was added and the reaction mixture was stirred at RT for 12 h. The progress of the reaction was monitored by TLC (M.Ph: 30% EtOAc in n-hexane). After completion of reaction, the reaction mixture was diluted with ice cold water. The aqueous layer was extracted with EtOAc (2×100 mL). The organic layer was washed thoroughly with cold water and brine, dried over anhydrous Na₂SO₄ and concentrated under reduced pressure. The crude compound was purified through prep HPLC to afford Example 33 (0.6 g, 54%) as an off-white solid. ¹H NMR (400 MHz, DMSO-d₆) δ 12.79 (s, 1H), 8.64 (d, J=4.0 Hz, 1H), 8.24 (t, J=6.4 Hz, 2H), 8.03 (d, J=8.0 Hz, 1H), 7.95-7.90 (m, 2H), 7.42-7.38 (m, 4H); LC-MS: m/z 417.07 [M+H]⁺; HPLC: 99.3%.

### Synthesis of Example 34

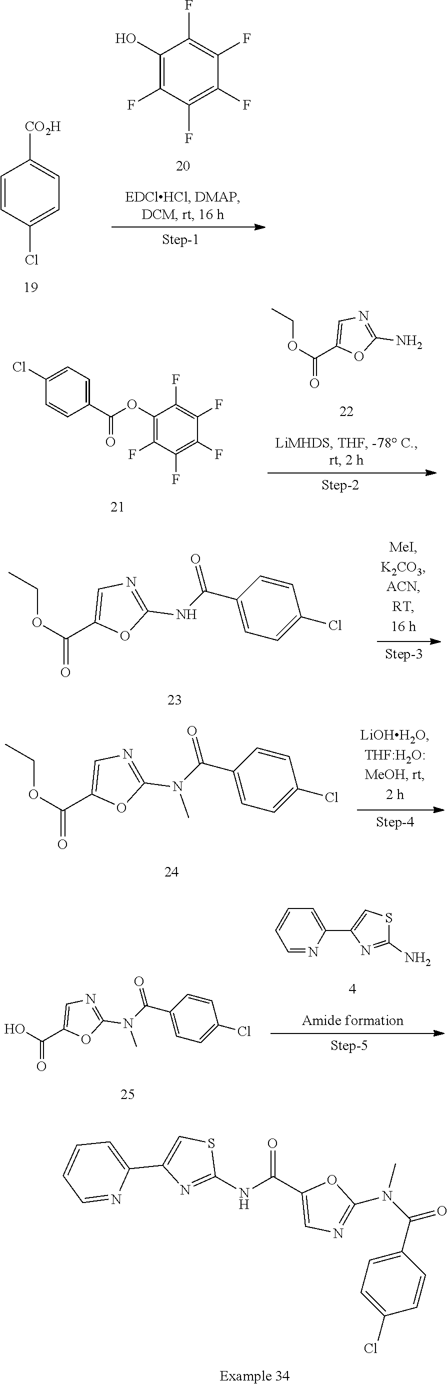

### Step-1: Perfluorophenyl 4-chlorobenzoate (21)

To a stirred solution of 4-chlorobenzoic acid (19, 1.0 g, 6.39, 1.0 eq) in dry DCM (50 mL) was added 2,3,4,5,6-pentafluorophenol (20, 1.30 g, 7.03 mmol, 1.1 eq) followed by EDCI.HCl (1.80 g, 9.5 mmol, 1.5 eq) and DMAP (156 mg, 1.28 mmol, 0.2 eq) at RT. The reaction mixture was stirred for 16 h. After the completion of reaction, the mixture was diluted with water, extracted with DCM. The organic layer was dried over Na₂SO₄ and solvent was evaporated under vacuum. The crude compound was purified through Silica gel column chromatography column chromatography by eluting with 5-10% EtOAc in heptane to afford perfluorophenyl 4-chlorobenzoate (21, 1.70 g, 82%) as a light-yellow sticky oil. ¹H NMR (400 MHz, DMSO-d₆): δ 8.20 (d, J=8.80 Hz, 2H), 7.75 (d, J=8.80 Hz, 2H); LC-MS: m/z 364.85 [M+H]⁺.

### Step-2: Ethyl 2-(4-chlorobenzamido)oxazole-5-carboxylate (23)

To a stirred solution of ethyl 2-aminooxazole-5-carboxylate (22, 300 mg, 1.92 mmol, 1.0 eq) in dry THE was added LiHMDS (1M soln in THF) (3.0 mL) at −78° C. After stirring for 15 min, then a solution of perfluorophenyl 4-chlorobenzoate (21, 743 mg, 2.31 mmol, 1.2 eq) was added dropwise over a period of 10 min. The reaction mixture was slowly warmed to RT and stirred for 2 h. After completion of the reaction, the reaction mixture was quenched with saturated NH₄Cl at 0° C. and extracted with EtOAc, washed with brine, dried over Na₂SO₄ and solvent was evaporated under reduced pressure. The crude compound was purified through column chromatography using 5% MeOH in DCM to afford ethyl 2-(4-chlorobenzamido)oxazole-5-carboxylate (23, 450 mg, 80%) as a light yellow solid. ¹H NMR (400 MHz, DMSO-d₆): δ 12.21 (br s, 1H), 7.99 (d, J=8.8 Hz, 2H), 7.92 (s, 1H), 7.57 (d, J=8.4 Hz, 2H), 4.32 (q, J=6.80 Hz, 2H), 1.28 (t, J=6.80 Hz, 3H); LC-MS: m/z 294.80 [M+H]⁺.

### Step-3: Ethyl 2-(4-chloro-N-methylbenzamido)oxazole-5-carboxylate (24)

To a solution of ethyl 2-(4-chlorobenzamido)oxazole-5-carboxylate (23, 400 mg, 1.36 mmol, 1.0 eq) in ACN (50 mL) was added K₂CO₃ (563 mg, 4.0 mmol, 3.0 eq) followed by Mel (0.85 mL, 13.6 mmol, 10.0 eq) at RT. The reaction mixture was stirred for 16 h. After the completion of the reaction, mixture was filtered through celite and filtrate was concentrated to dryness. The crude mixture was purified by Silica gel column chromatography by eluting with 1-2% MeOH in DCM to afford ethyl 2-(4-chloro-N-methylbenzamido)oxazole-5-carboxylate (24, 20 mg, 5%) as an off-white solid. ¹H NMR (400 MHz, DMSO-d₆): δ 8.38 (s, 1H), 8.13 (d, J=8.4 Hz, 2H), 7.52 (d, J=8.0 Hz, 2H), 4.32 (q, J=6.80 Hz, 2H), 3.47 (s, 3H), 1.28 (t, J=7.20 Hz, 3H); LC-MS: m/z 308.95 [M+H]⁺.

### Step-4: 2-(4-chloro-N-methylbenzamido)oxazole-5-carboxylic acid (25)

To a solution of ethyl 2-(4-chloro-N-methylbenzamido)oxazole-5-carboxylate (24, 10 mg, 0.032 mmol, 1.0 eq) in a mixture THF:H₂O (2.5 mL) (4:1 mL) was added aqueous solution of LiOH·H₂O (2 mg, 0.05 mmol, 1.5 eq) at 0° C. The reaction mixture was stirred for 2 h at RT.

After completion of the reaction, solvent was evaporated and then diluted with water (0.5 mL) and acidified with 1N HCl to bring pH-2 and extracted with EtOAc (2.0 mL) and concentrated to give 25 as an off-white powder. LC-MS: m/z 280.80 [M+H]⁺.

### Step-5: Example 34

In line with General Procedure A, compound 25 was coupled with 4-(pyridin-2-yl)thiazol-2-amine (4) to afford Example 34 as an off-white solid. ¹H NMR (400 MHz, DMSO-d₆) δ 13.00 (s, 1H), 8.61 (bs, 1H), 8.03 (s, 1H), 7.99 (d, J=7.8 Hz, 1H), 7.91-7.88 (m, 2H), 7.49 (bs, 4H), 7.34 (t, J=8.8 Hz, 2H), 3.52 (s, 3H); LC-MS: m/z 440.0 [M+H]⁺; HPLC: 96.2%.

### Synthesis of Example 35

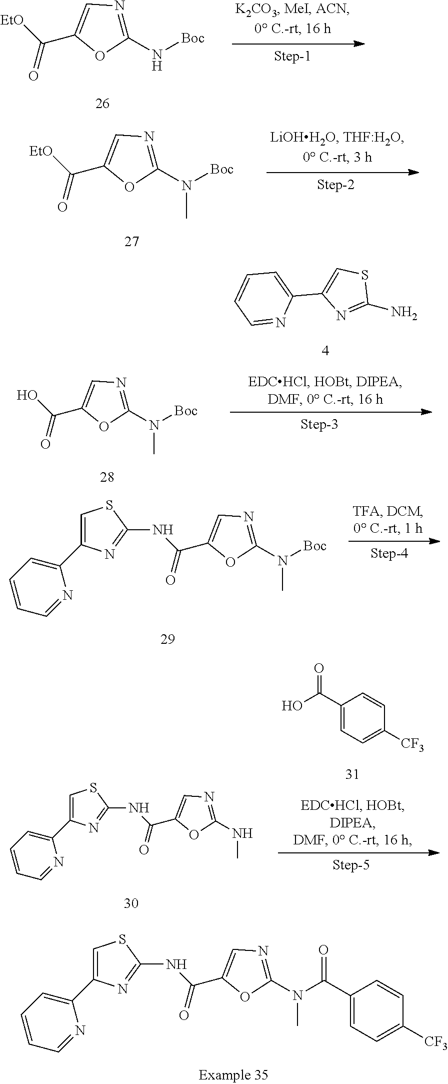

### Step-1: Ethyl 2-((tert-butoxycarbonyl)(methyl)amino)oxazole-5-carboxylate (27)

To a solution of ethyl 2-((tert-butoxycarbonyl)amino)oxazole-5-carboxylate (26, 2.0, 7.80 mmol, 1.0 eq) in ACN (20 mL) was added K₂CO₃ (3.23 g, 23.40 mmol, 3.0 eq) at RT. After stirring for 15 min, Mel (5.50 g, 39 mmol, 5.0 eq) was added to the reaction mixture at 0° C. and stirred for 16 h at RT. After the completion of reaction, the reaction mixture was filtered, and filtrate was concentrated. The resulting crude compound was purified through Silica gel column chromatography using 10% EtOAc in Hexane to afford ethyl 2-((tert-butoxycarbonyl)(methyl)amino)oxazole-5-carboxylate (27, 1.70 g, 83%) as a colorless liquid. ¹H NMR (DMSO-d₆, 400 MHz): δ 7.64 (s, 1H), 4.36 (q, J=6.80 Hz, 2H), 3.41 (s, 3H), 1.38 (t, J=5.20 Hz, 5 3H), 1.55 (s, 9H); LC-MS: m/z 215.15 [M-56]⁺.

Step-2: 2-((tert-butoxycarbonyl)(methyl)amino)oxazole-5-carboxylic acid (28)

To a solution of ethyl 2-((tert-butoxycarbonyl)(methyl)amino)oxazole-5-carboxylate (28, 1.0 g, 3.70 mmol, 1.0 eq) in a mixture of THF:H₂O (2:1) (15 mL) was added an aqueous solution of LiOH·H₂O (0.23 g, 5.55 mmol, 1.50 eq) at 0° C. The reaction mixture was stirred at 10 RT for 3 h. After completion of the reaction, solvent was evaporated under rotatory evaporation.

The resulting crude residue was diluted with water and acidified with aq. HCl (pH˜4) and extracted with 5% MeOH in DCM. The combined organic layer was dried over Na₂SO₄ and concentrated. The crude compound was purified through trituration with n-pentane to afford 2-((tert-butoxycarbonyl)(methyl)amino)oxazole-5-carboxylic acid (28, 0.55 g, 62%) as an off-white solid. ¹H NMR (DMSO-d₆, 400 MHz): δ 13.45 (br s, 1H), 7.75 (s, 1H), 3.28 (s, 3H), 1.47 (s, 9H); LC-MS: m/z 241.05 [M+H]⁺.

### Step-3: Tert-butyl methyl(5-((4-(pyridin-2-yl)thiazol-2-yl)carbamoyl)oxazol-2-yl)carbamate (29)

To a solution of 2-((tert-butoxycarbonyl)(methyl)amino)oxazole-5-carboxylic acid (28, 550 mg, 2.27 mmol, 1.0 eq) in DMF (10 mL) was added EDC.HCl (652 mg, 3.4 mmol, 1.5 eq), HOBt (459 mg, 3.4 mmol, 1.5 eq) and DIPEA (1.18 mL, 6.81 mmol, 3.0 eq) at 0° C. under nitrogen atmosphere. After stirring for 15 min, 4-(pyridin-2-yl)thiazol-2-amine (4, 401 mg, 2.27 mmol, 1.0 eq) was added to the reaction mixture and stirred at RT for 16 h. Progress of the reaction was monitored by TLC and LCMS. After completion of the reaction, solvent was evaporated. The crude mixture was diluted with ice-cold water and extracted 10% MeOH in DCM. The combined organic layer was dried over Na₂S04 and concentrated under reduced pressure. The resulting crude compound was purified through Silica gel column chromatography (10% MeOH in DCM) to afford tert-butyl methyl(5-((4-(pyridin-2-yl)thiazol-2-yl)carbamoyl)oxazol-2-yl)carbamate (29, 400 mg, 44%) as an off-white solid. ¹H NMR (DMSO-d₆, 400 MHz): δ 12.93 (s, 1H), 8.60 (d, J=4.40 Hz, 1H), 8.23 (s, 1H), 8.0 (d, J=7.60 Hz, 1H), 7.92-7.90 (m, 1H), 7.89 (s, 1H), 7.37-7.34 (m, 1H), 3.3.3 (s, 3H), 1.51 (s, 9H); LC-MS: m/z 402.05 [M+H]⁺.

### Step-4: 2-(Methylamino)-N-(4-(pyridin-2-yl)thiazol-2-yl)oxazole-5-carboxamide (30)

To a solution of tert-butyl methyl(5-((4-(pyridin-2-yl)thiazol-2-yl)carbamoyl)oxazol-2-yl)carbamate (29, 400 mg, 0.99 mmol, 1.0 eq) in DCM (5 mL) wad added TFA (5 mL) at 0° C. under nitrogen atmosphere. The reaction mixture was stirred at RT for 1 h. The progress of the reaction was monitored by TLC. After the completion of reaction, excess of TFA was removed under reduced pressure. The crude mixture was dissolved into DCM and washed with saturated NaHCO₃ solution, dried over Na₂SO₄ and concentrated under rotatory to get 2-(methylamino)-N-(4-(pyridin-2-yl)thiazol-2-yl)oxazole-5-carboxamide (30, 280 mg, 93%) as a white solid. ¹H NMR (DMSO-d₆, 400 MHz): δ 12.50 (s, 1H), 8.59 (d, J=4.80 Hz, 1H), 8.06 (s, 1H), 8.02 (d, J=5.20 Hz, 1H), 7.97 (d, J=8.80 Hz, 1H), 7.90-7.85 (m, 1H), 7.82 (s, 1H), 7.35-7.33 (m, 1H), 2.85 (d, J=4.40 Hz, 3H); LC-MS: m/z 301.90 [M+H]⁺.

### Step-5: Example 35

A solution of 4-trifluoromethylbenzoic acid (30) (1 eq), EDC-HCl (1.5 eq), HOBt (1.5 eq) and DIPEA (3 eq) in DMF was stirred at rt for 10 min. To this was added a solution of 30 in DMF and reaction mixture was stirred at rt for 16 h. Progress of reaction was monitored by TLC.

After completion, the reaction mixture was quenched with H₂O and aqueous layer was extracted with EtOAc. Combined organic layers were washed with H₂O and brine solution, dried over anhydrous Na₂S₂O₄ and concentrated. The crude obtained was purified by column chromatography to afford Example 35. ¹H NMR (400 MHz, DMSO-d6) δ 12.54 (s, 1H), 8.61 (bs, 1H), 8.03 (s, 1H), 7.95 (d, J=7.8 Hz, 1H), 7.94-7.88 (m, 2H), 7.80 (d, J=6.8 Hz, 2H), 7.69 (d, J=8.8 Hz, 2H), 7.34 (t, J=8.8 Hz, 2H), 3.54 (s, 3H); LC-MS: m/z 474.1 [M+H]⁺; HPLC: 99.5%.

### Synthesis of Example 36

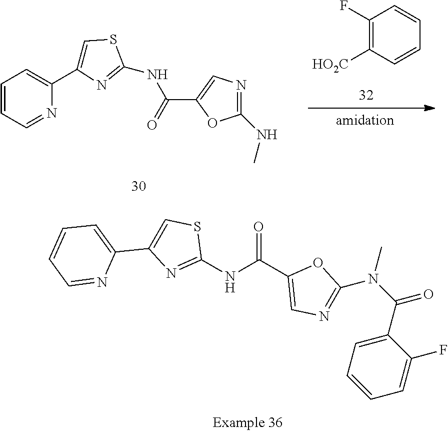 [SMILES: CNc1ncc(C(=O)Nc2nc(-c3ccccn3)cs2)o1 ; O=C(O)c1ccccc1F ; CN(C(=O)c1ccccc1F)c1ncc(C(=O)Nc2nc(-c3ccccn3)cs2)o1]

Using reaction conditions similar to that used for Step-3 (Example 35), intermediate 30 was coupled with 2-fluorobenzoic acid (32) to provide Example 36. ¹H NMR (400 MHz, DMSO-d6) δ 12.98 (s, 1H), 8.62 (d, J=4.4 Hz, 1H), 8.03 (s, 1H) 7.99 (d, J=8.0 Hz, 1H), 7.91-7.87 (m, 2H), 7.58-7.53 (m, 2H), 7.36 (t, J=6.4 Hz, 1H), 7.31-7.21 (m, 2H), 3.56 (s, 3H); LC-MS: m/z 424.0 [M+H]⁺; HPLC: 99.2%.

### Synthesis of Example 37

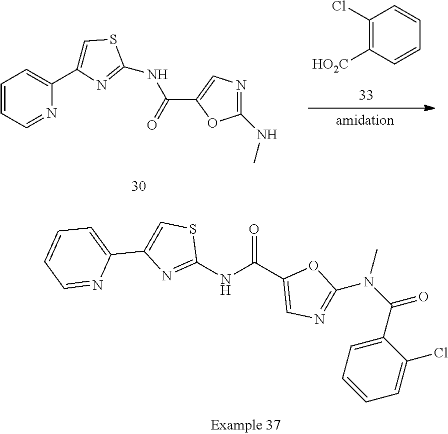 [SMILES: CNc1ncc(C(=O)Nc2nc(-c3ccccn3)cs2)o1 ; O=C(O)c1ccccc1Cl ; CN(C(=O)c1ccccc1Cl)c1ncc(C(=O)Nc2nc(-c3ccccn3)cs2)o1]

Using reaction conditions similar to that used for Step-5 (Example 35), intermediate 30 was coupled with 2-chlorobenzoic acid (33) to provide Example 37. ¹H NMR (400 MHz, DMSO-d6) δ 12.93 (s, 1H), 8.62 (d, J=4.4 Hz, 1H), 8.00-7.91 (m, 2H), 7.89 (t, J=7.8 Hz, 2H) 7.50-7.33 (m, 5H), 3.56 (s, 3H); LC-MS: m/z 440.0 [M+H]⁺; HPLC: 98.7%.

### Synthesis of Example 38

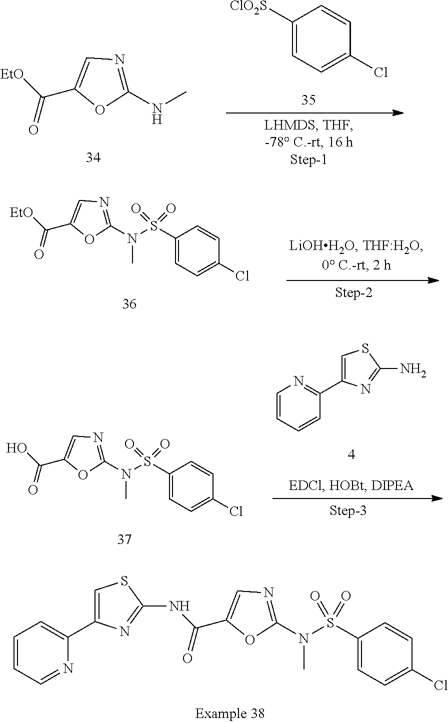 [SMILES: CCOC(=O)c1cnc(NC)o1 ; O=S(=O)(Cl)c1ccc(Cl)cc1 ; CCOC(=O)c1cnc(N(C)S(=O)(=O)c2ccc(Cl)cc2)o1 ; Nc1nc(-c2ccccn2)cs1 ; CN(c1ncc(C(=O)O)o1)S(=O)(=O)c1ccc(Cl)cc1 ; CN(c1ncc(C(=O)Nc2nc(-c3ccccn3)cs2)o1)S(=O)(=O)c1ccc(Cl)cc1]

### Step-1: Ethyl 2-((4-chloro-N-methylphenyl)sulfonamido)oxazole-5-carboxylate (36)

To a solution of ethyl 2-(methylamino)oxazole-5-carboxylate (34, 150 mg, 0.88 mmol, 1.0 eq) in anhydrous THE (5 mL) was added 1M solution of LiHMDS (0.4 mL, 1.05 mmol, 1.2 eq) over a period of 15 min at −78° C. under nitrogen atmosphere. Then, a solution of 4-chlorobenzenesulfonyl chloride (35, 75 mg, 1.05 mmol, 1.2 eq) in THE was added dropwise at the same temperature and stirred for 2 h at −78° C. followed by slowly warming to RT. After the completion of reaction, the reaction mixture was cool to 0° C. and quenched with saturated NH₄Cl and extracted with EtOAc. The combined organic layer was dried over Na₂SO₄ and concentrated. The resulting crude compound was purified through Silica gel column chromatography (15% EtOAc in Hexane) to afford ethyl 2-((4-chloro-N-methylphenyl)sulfonamido)oxazole-5-carboxylate (36, 290 mg, 95%) as a colorless thick liquid. ¹H NMR (DMSO-d₆, 400 MHz): δ 8.02 (d, J=8.40 Hz, 2H), 7.90 (s, 1H), 7.75 (d, J=8.40 Hz, 2H), 4.30 (q, J=7.20 Hz, 2H), 3.44 (s, 3H), 1.29 (t, J=7.20 Hz, 3H); LC-MS: m/z 344.80 [M+H]⁺.

### Step-2: 2-((4-chloro-N-methylphenyl)sulfonamido)oxazole-5-carboxylic acid (37)

To a solution of ethyl 2-((4-chloro-N-methylphenyl)sulfonamido)oxazole-5-carboxylate (36, 340 mg, 0.98 mmol, 1.0 eq) in a mixture of THF:H₂O (2:1) (15 mL) was added an aqueous solution of LiOH·H₂O (46 mg, 1.08 mmol, 1.10 eq) at 0° C. The reaction mixture was stirred at RT for 2 h. After completion of the reaction, solvent was evaporated under rotatory. The resulting crude residue was diluted with water and acidified with aq. HCl (pH˜4) to get white precipitate which was filtered and dried under vacuum to afford 2-((4-chloro-N-methylphenyl)sulfonamido)oxazole-5-carboxylic acid (37, 240 mg, 83%) as a white solid. ¹H NMR (DMSO-d₆, 400 MHz): δ 13.48 (br s, 1H), 7.97 (d, J=8.80 Hz, 2H), 7.76 (s, 1H), 7.75 (d, J=8.80 Hz, 2H), 3.41 (s, 3H); LC-MS: m/z 316.85 [M+H]⁺.

### Step-3: Example 38

To a stirred mixture of 2-((4-chloro-N-methylphenyl)sulfonamido)oxazole-5-carboxylic acid (37) (100 mg, 0.31 mmol, 1 eq) in DCM (10 mL), EDCl (90.5 mg, 0.47 mmol, 1.5 eq), HOBt (63.7 mg, 0.47 mmol, 1.5 eq) and DIPEA (0.16 mL, 0.94 mmol, 3 eq) were added at RT. The mixture was stirred at same temperature for 30 min. Then 4-(pyridin-2-yl)thiazol-2-amine (4) (55.8 mg, 0.31 mmol, 1 eq) was added into the reaction mixture at 0-5° C. and allowed to stirred at RT for 12 h. The progress of the reaction was monitored by TLC (M.Ph: 5% MeOH in DCM). The reaction mixture was diluted with water (70 mL) and extracted with EtOAc (2×50 mL). The combined organic layers were washed with brine, dried over sodium sulfate, filtered and concentrated to dryness. The crude was purified through silica gel column chromatography (elution: 10-50% EtOAc in n-hexane) to afford Example 38 (40 mg, 11%) as an off-white solid. ¹H NMR (DMSO-d₆, 400 MHz): δ 12.98 (s, 1H), 8.61 (bs, 1H), 8.13 (s, 1H), 8.06 (d, J=7.8 Hz, 1H), 8.04 (d, J=8.8 Hz, 1H), 7.97-7.78 (m, 3H), 7.79 (d, J=6.8 Hz, 2H), 7.34 (t, J=8.8 Hz, 2H), 3.50 (s, 3H); LC-MS: m/z 476.00 [M+H]⁺; HPLC: 99.6%.

### Synthesis of Example 39

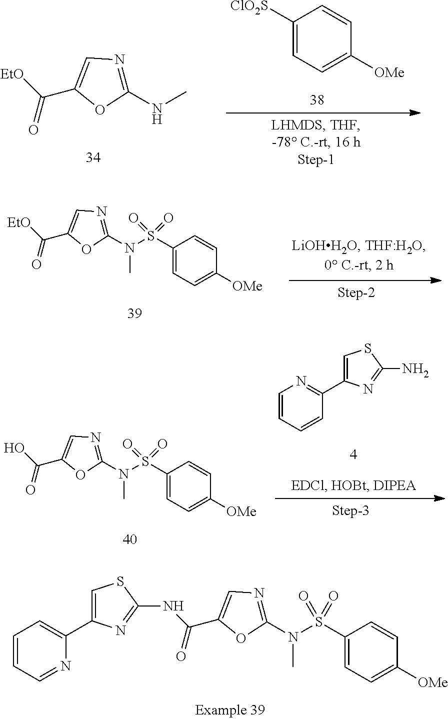 [SMILES: CCOC(=O)c1cnc(NC)o1 ; COc1ccc(S(=O)(=O)Cl)cc1 ; CCOC(=O)c1cnc(N(C)S(=O)(=O)c2ccc(OC)cc2)o1 ; Nc1nc(-c2ccccn2)cs1 ; COc1ccc(S(=O)(=O)N(C)c2ncc(C(=O)O)o2)cc1 ; COc1ccc(S(=O)(=O)N(C)c2ncc(C(=O)Nc3nc(-c4ccccn4)cs3)o2)cc1]

### Step-1: Ethyl 2-((4-methoxy-N-methylphenyl)sulfonamido)oxazole-5-carboxylate (39)

To a solution of ethyl 2-(methylamino)oxazole-5-carboxylate (34, 380 mg, 2.23 mmol, 1.0 eq) was added LiHMDS (1M in THF) (3.30 mL3.35 mmol, 1.5 eq) at −78° C. After stirring for 30 min, a stock solution of 4-methoxybenzenesulfonyl chloride (38, 460 mg, 2.23 mmol, 1.0 eq) was added dropwise over a period of 20 min and slowly allowed to be stirred for 16 h at RT.

After completion of reaction, the reaction mixture was cooled to 0° C. and to this, saturated NH₄Cl was added slowly and extracted with EtOAc and dried over Na₂SO₄ and concentrated.

The crude mixture was isolated through combi-flash column chromatography by eluting with 40% EtOAc in heptane to afford ethyl 2-((4-methoxy-N-methylphenyl)sulfonamido)oxazole-5-carboxylate (39, 270 mg, 36%) as a colorless sticky compound. ¹H NMR (400 MHz, DMSO-d₆): δ 8.01 (d, J=8.80 Hz, 2H), 7.57 (s, 1H), 7.00 (d, J=8.8 Hz, 2H), 4.34 (q, J=7.2 Hz, 2H), 3.89 (s, 3H), 3.53 (s, 3H), 1.39 (t, J=7.2 Hz, 3H); LC-MS: m/z 341.0 [M+H]⁺.

### Step-2: 2-((4-methoxy-N-methylphenyl)sulfonamido)oxazole-5-carboxylic acid (40)

To a solution of ethyl 2-((4-methoxy-N-methylphenyl)sulfonamido)oxazole-5-carboxylate (39, 270 mg, 0.79 mmol, 1.0 eq) in a mixture THF:H₂O:MeOH (10:1:1) was added aqueous solution of LiOH·H₂O (66 mg, 1.6 mmol, 2.0 eq) at 0° C. The reaction mixture was stirred for 3 h at RT. Progress of reaction was monitored by TLC. After completion of reaction, solvent was evaporated and residue was diluted with water and acidified with 6N HCl to bring pH-3 which was extracted with EtOAc, washed with brine, dried over Na₂SO₄ and solvent was evaporated to get 2-((4-methoxy-N-methylphenyl)sulfonamido)oxazole-5-carboxylic acid (40, 220 mg crude) as a white powder. ¹H NMR (400 MHz, DMSO-d₆): δ 13.42 (br s, 1H), 7.89 (d, J=8.80 Hz, 2H), 7.70 (s, 1H), 7.16 (d, J=8.8 Hz, 2H), 3.85 (s, 3H), 2.67 (s, 3H); LC-MS: m/z 313.0 [M+H]⁺.

### Step-3: Example 39

Using reaction conditions similar to those described above for Example 38/Step-3, intermediate acid 40 was coupled with amine 4 to afford Example 39 as an off-white solid. ¹H NMR (DMSO-d₆, 400 MHz): δ 12.95 (s, 1H), 8.60 (d, J=4.4 Hz, 1H), 8.14 (s, 1H), 7.95-7.99 (m, 3H), 7.88-7.91 (m, 2H), 7.35 (t, J=6.0 Hz, 1H), 7.19 (d, J=8.8 Hz, 2H), 3.80 (s, 3H), 3.46 (s, 3H); LC-MS: m/z 472.1 [M+H]⁺; HPLC: 98.8%.

### Synthesis of Example 40

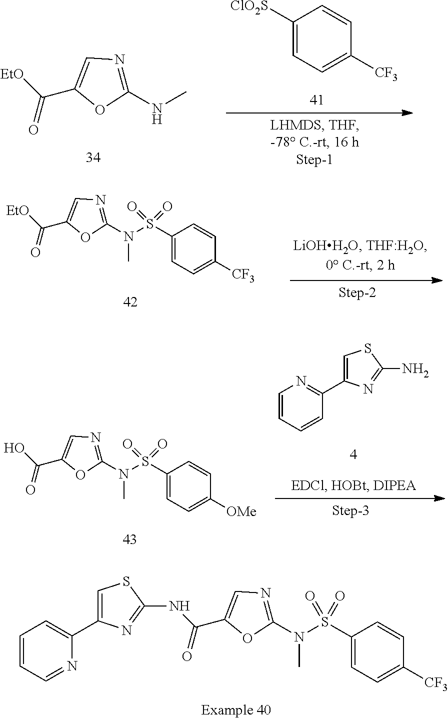 [SMILES: CCOC(=O)c1cnc(NC)o1 ; O=S(=O)(Cl)c1ccc(C(F)(F)F)cc1 ; CCOC(=O)c1cnc(N(C)S(=O)(=O)c2ccc(C(F)(F)F)cc2)o1 ; Nc1nc(-c2ccccn2)cs1 ; COc1ccc(S(=O)(=O)N(C)c2ncc(C(=O)O)o2)cc1 ; CN(c1ncc(C(=O)Nc2nc(-c3ccccn3)cs2)o1)S(=O)(=O)c1ccc(C(F)(F)F)cc1]

### Step-1: Ethyl 2-((N-methyl-4-(trifluoromethyl)phenyl)sulfonamido)oxazole-5-carboxylate (42)

To a solution of ethyl 2-(methylamino)oxazole-5-carboxylate (34, 200 mg, 1.18 mmol, 1.0 eq) in anhydrous THE (20 mL) was added 4-(trifluoromethyl)benzenesulfonyl chloride (41, 3.23 g, 1.53 mmol, 1.3 eq) and 1M solution of LiHMDS (2 mL, 1.53 mmol, 1.8 eq) over a period of 30 min at −78° C. under inert atmosphere. The reaction mixture was stirred for 2 h at −78° C. followed by slowly warming to RT. After the completion of reaction, the reaction mixture was cooled to 0° C. and quenched with saturated NH₄Cl and extracted with EtOAc. The combined organic layer was dried over Na₂SO₄ and concentrated. The resulting crude compound was purified through Silica gel column chromatography (5% EtOAc in Hexane) to afford ethyl 2-((N-methyl-4-(trifluoromethyl)phenyl)sulfonamido)oxazole-5-carboxylate (42, 310 mg, 70%) as a pale yellow oil. ¹H NMR (DMSO-d₆, 400 MHz): δ 8.22 (d, J=11.60 Hz, 2H), 8.07 (d, J=8.80 Hz, 2H), 7.89 (s, 1H), 4.30 (q, J=7.20 Hz, 2H), 3.47 (s, 3H), 1.28 (t, J=6.80 Hz, 3H); LC-MS: m/z 378.90 [M+H]⁺.

### Step-2: 2-((N-methyl-4-(trifluoromethyl)phenyl)sulfonamido)oxazole-5-carboxylic acid (43)

To a solution of ethyl 2-((N-methyl-4-(trifluoromethyl)phenyl)sulfonamido)oxazole-5-carboxylate (42, 320 mg, 0.85 mmol, 1.0 eq) in a mixture of THF:H₂O (2:1) (20 mL) was added an aqueous solution of LiOH·H₂O (71 mg, 1.69 mmol, 2.0 eq) at 0° C. The reaction mixture was stirred at RT for 2 h. After completion of the reaction, solvent was evaporated under rotatory. The resulting crude residue was diluted with water and acidified with aq. HCl (pH˜4) and extracted with 5% MeOH in DCM. The combined organic layer was dried over Na₂SO₄ and concentrated. The crude compound was purified through trituration with n-pentane to afford 2-((N-methyl-4-(trifluoromethyl)phenyl)sulfonamido)-oxazole-5-carboxylic acid (43, 240 mg, 81%) as an off-white solid. ¹H NMR (DMSO-d₆, 400 MHz): δ 13.49 (br s, 1H), 8.20 (d, J=8.0 Hz, 2H), 8.05 (d, J=7.60 Hz, 2H), 7.80 (s, 1H), 3.45 (s, 3H); LC-MS: m/z 350.90 [M+H]⁺.

### Step-3: Example 40

Using reaction conditions similar to those described above for Example 38/Step-3, intermediate acid 40 was coupled with amine 4 to afford Example 40 as an off-white solid. ¹H NMR (DMSO-d₆, 400 MHz): δ 13.01 (s, 1H), 8.61 (bs, 1H), 8.27 (d, J=7.8 Hz, 2H), 8.20-7.87 (m, 6H), 7.35 (m, 1H), 3.55 (s, 3H); LC-MS: m/z 510.1 [M+H]⁺; HPLC: 99.0%.

### Synthesis of Example 41

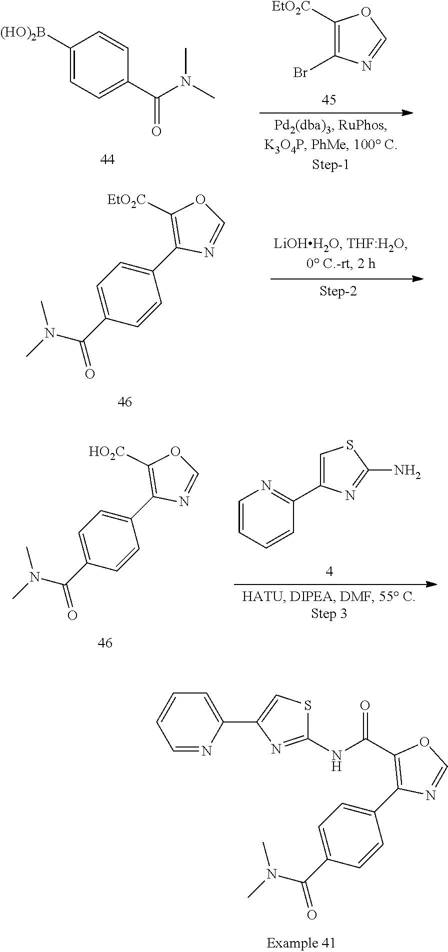 [SMILES: CN(C)C(=O)c1ccc(B(O)O)cc1 ; CCOC(=O)c1ocnc1Br ; CCOC(=O)c1ocnc1-c1ccc(C(=O)N(C)C)cc1 ; CN(C)C(=O)c1ccc(-c2ncoc2C(=O)O)cc1 ; Nc1nc(-c2ccccn2)cs1 ; CN(C)C(=O)c1ccc(-c2ncoc2C(=O)Nc2nc(-c3ccccn3)cs2)cc1]

### Step-1: Ethyl 4-(4-(dimethylamino-formyl)-phenyl)-oxazole-5-carboxylate (46)

In a microwave vial equipped with a magnetic stir bar was charged with ethyl 4-bromo-1,3-oxazole-5-carboxylate (45) (500 mg, 0.002 mol, 1.0 eq), Pd₂(dba)₃ (83.2 mg, 0.04 eq), RuPhos (84.8 mg, 0.08 eq), and (4-boronic acid-phenyl)-dimethylamino-methanone (44, 1.2 eq), anhydrous crushed K₃PO₄ (1.447 g, 0.007 mol, 3.0 eq). Dry toluene (11.4 mL) was added, and the vial capped with a rubber septum and purged with argon for 10 min. The reaction mixture was heated in an oil bath at 100° C. with vigorous stirring for 18 h. The reaction mixture was then allowed to cool to room temperature, diluted with EtOAc (50 mL), filtered through a pad of Celite, and the pad washed with EtOAc and the filtrate dried under reduced pressure. The crude material obtained was purified by normal phase chromatography to afford intermediate 46 which was used directly in the next step.

### Step-2: 4-(4-(dimethylamino-formyl)-phenyl)-oxazole-5-carboxylic acid (47)

To a solution of intermediate ester (46, 400 mg, 0.001 mol, 1.0 eq) in a mixture of THF (6.6 mL) and MeOH (0.31 mL) was added a solution of LiOH·H₂O (42 mg, 4.0 eq) in water (8.5 mL) at rt. The reaction mixture was stirred at RT for 16 h. After completion of the reaction the solution was cooled to 0° C. and acidified with aq. HCl (pH-3) and extracted with EtOAc. The combined organic layer was dried over Na₂SO₄ and concentrated to afford 47. The crude compound was used directly in the next step.

### Step-3: Example 41

In line with the General Procedure A, acid 47 (160 mg, 1 eqv) was coupled with 4-(pyridin-2-yl)thiazol-2-amine (4) (177 mg, 1.05 eq) to afford Example 41. ¹H NMR (400 MHz, DMSO-d₆) δ 8.82 (s, 1H), 8.62 (ddd, J=4.8, 1.8, 0.9 Hz, 1H), 8.25-8.18 (m, 2H), 8.01 (dt, J=7.9, 1.1 Hz, 1H), 7.96-7.86 (m, 2H), 7.58-7.50 (m, 2H), 7.35 (ddd, J=7.5, 4.7, 1.3 Hz, 1H), 3.00 (s, 3H), 2.94 (s, 3H), NH not observed; LC-MS: m/z 420.1 [M+H]⁺.

### Synthesis of Example 42

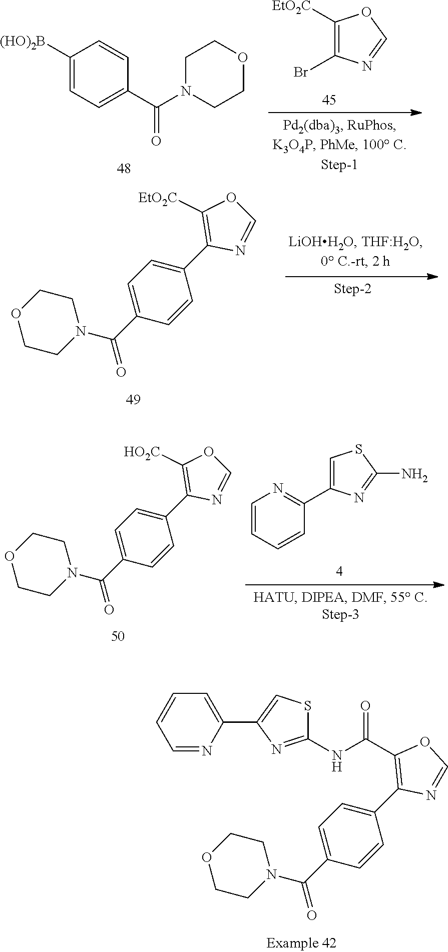

### Step-1: Ethyl 4-(4-(dimethylamino-formyl)-phenyl)-oxazole-5-carboxylate (49)

In a microwave vial equipped with a magnetic stir bar was charged with ethyl 4-bromo-1,3-oxazole-5-carboxylate (45) (300 mg, 0.002 mol, 1.0 eq), Pd₂(dba)₃ (50 mg, 0.04 eq), RuPhos (50.8 mg, 0.08 eq) and (4-boronic acid-phenyl)-(morpholin-4-yl)-methanone (48, 1.2 eqv) and anhydrous crushed K₃PO₄ (868 mg, 4.09 mmol, 3.0 eq). Dry toluene (3 mL) was added, and the vial capped with a rubber septum and purged with argon for 10 min. The reaction mixture was heated in an oil bath at 100° C. with vigorous stirring for 18 h. The reaction mixture was then allowed to cool to room temperature, diluted with EtOAc (50 mL), filtered through a pad of Celite, and the pad washed with EtOAc and the filtrate dried under reduced pressure. The crude material obtained was purified by normal phase chromatography using 0-50% EtOAc in n-hexane to afford intermediate 49 which was used directly in the next step.

### Step-2: 4-(4-(dimethylamino-formyl)-phenyl)-oxazole-5-carboxylic acid (50)

To a solution of intermediate ester (49, 259 mg, 0.784 mmol, 1.0 eq) in a mixture of THF (2 mL) was added a solution of LiOH·H₂O (132 mg, 4.0 eq) in water (4 mL) at rt. The reaction mixture was stirred at RT for 16 h. After completion of the reaction the solution was cooled to 0° C. and acidified with aq. HCl (pH-3) and extracted with EtOAc. The combined organic layer was dried over Na₂SO₄ and concentrated to afford 50. The crude compound was used directly in the next step.

### Step-3: Example 42

In line with the General Procedure A, acid 50 (160 mg, 1 eq) was coupled with 4-(pyridin-2-yl)thiazol-2-amine (4) (177 mg, 1.05 eq) to afford Example 42. ¹H NMR (400 MHz, DMSO-d₆) δ 8.82 (s, 1H), 8.62 (ddd, J=4.8, 1.8, 0.9 Hz, 1H), 8.24-8.22 (m, 1H), 8.04-7.98 (m, 1H), 7.95-7.87 (m, 3H), 7.58-7.52 (m, 2H), 7.35 (ddd, J=7.5, 4.8, 1.3 Hz, 1H), 3.70-3.52 (m, 8H), NH not observed; LC-MS: m/z 462.1 [M+H]⁺.

### Synthesis of Example 43

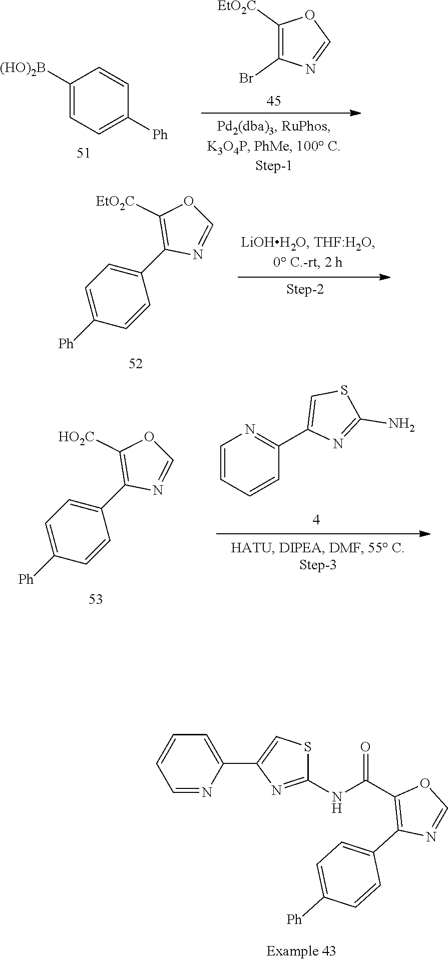 [SMILES: OB(O)c1ccc(-c2ccccc2)cc1 ; CCOC(=O)c1ocnc1Br ; CCOC(=O)c1ocnc1-c1ccc(-c2ccccc2)cc1 ; O=C(O)c1ocnc1-c1ccc(-c2ccccc2)cc1 ; Nc1nc(-c2ccccn2)cs1 ; O=C(Nc1nc(-c2ccccn2)cs1)c1ocnc1-c1ccc(-c2ccccc2)cc1]

### Step-1: Ethyl 4-(4-(dimethylamino-formyl)-phenyl)-oxazole-5-carboxylate (52)

In a microwave vial equipped with a magnetic stir bar was charged with ethyl 4-bromo-1,3-oxazole-5-carboxylate (45) (300 mg, 0.002 mol, 1.0 eq), Pd₂(dba)₃ (50 mg, 0.04 eq), RuPhos (50.8 mg, 0.08 eq) and 4-phenyl-phenyl boronic acid (51, 381 mg, 1.2 eq) anhydrous crushed K₃PO₄ (868 mg, 4.09 mmol, 3.0 eq). Dry toluene (3 mL) was added, and the vial capped with a rubber septum and purged with argon for 10 min. The reaction mixture was heated in an oil bath at 100° C. with vigorous stirring for 1.5 h. The reaction mixture was then allowed to cool to room temperature, diluted with EtOAc (50 mL), filtered through a pad of Celite, and the pad washed with EtOAc and the filtrate dried under reduced pressure. The crude material obtained was purified by normal phase chromatography using 0-50% EtOAc in n-hexane to afford intermediate 52 which was used directly in the next step.

### Step-2: 4-(4-(dimethylamino-formyl)-phenyl)-oxazole-5-carboxylic acid (53)

To a solution of intermediate ester (52, 260 mg, 0.793 mmol, 1.0 eq) in a mixture of THF (2 mL) was added a solution of LiOH·H₂O (133 mg, 4.0 eq) in water (4 mL) at rt. The reaction mixture was stirred at RT for 16 h. After completion of the reaction the solution was cooled to 0° C. and acidified with aq. HCl (pH-2) and extracted with EtOAc. The combined organic layer was dried over Na₂SO₄ and concentrated to afford 53. The crude compound was used directly in the next step.

### Step-3: Example 43

In line with the General Procedure A, acid 53 (236 mg, 1 eq) was coupled with 4-(pyridin-2-yl)thiazol-2-amine (4) (154 mg, 1.1 eq) to afford Example 43. ¹H NMR (400 MHz, DMSO-d₆) δ 8.82 (s, 1H), 8.81 (s, 1H), 8.62 (ddd, J=4.8, 1.8, 0.9 Hz, 1H), 8.31-8.25 (m, 2H), 8.02 (dt, J=8.0, 1.1 Hz, 1H), 7.96-7.81 (m, 4H), 7.84-7.73 (m, 2H), 7.58-7.48 (m, 2H), 7.35 (ddd, J=7.5, 4.7, 1.2 Hz, 1H), NH not observed; LC-MS: m/z 459.1 [M+H]⁺.

### Synthesis of Example 44

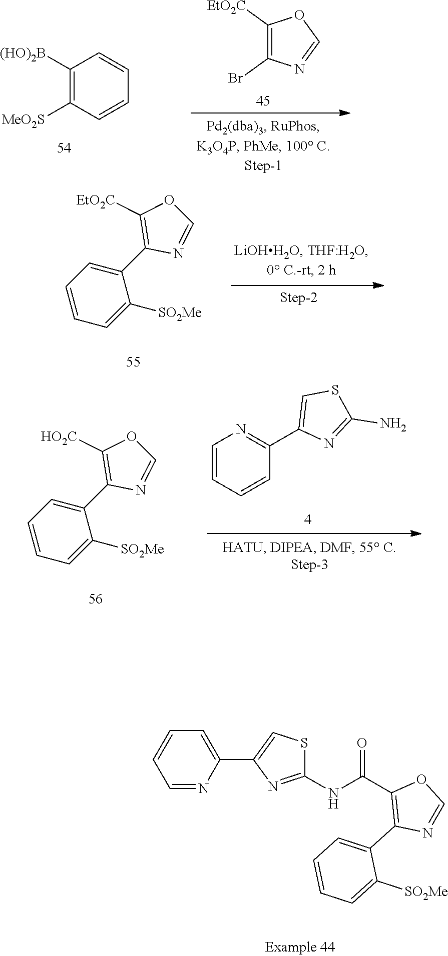 [SMILES: CS(=O)(=O)c1ccccc1B(O)O ; CCOC(=O)c1ocnc1Br ; CCOC(=O)c1ocnc1-c1ccccc1S(C)(=O)=O ; CS(=O)(=O)c1ccccc1-c1ncoc1C(=O)O ; Nc1nc(-c2ccccn2)cs1 ; CS(=O)(=O)c1ccccc1-c1ncoc1C(=O)Nc1nc(-c2ccccn2)cs1]

### Step-1: Ethyl 4-(2-methylsulfonyl-phenyl)-oxazole-5-carboxylate (55)

In a microwave vial equipped with a magnetic stir bar was charged with ethyl 4-bromo-1,3-oxazole-5-carboxylate (45) (250 mg, 1.14 mmol, 1.0 eq), Pd₂(dba)₃ (41.6 mg, 0.04 eq), RuPhos (42.4 mg, 0.08 eq) and 2-methylsulfonyl-phenyl boronic acid (54, 273 mg, 1.364 mmol, 1.2 eq) anhydrous crushed K₃PO₄ (724 mg, 3.41 mmol, 3.0 eq). Dry toluene (2.5 mL) was added, and the vial capped with a rubber septum and purged with argon for 10 min. The reaction mixture was heated in an oil bath at 100° C. with vigorous stirring for 1.5 h. The reaction mixture was then allowed to cool to room temperature, diluted with EtOAc (50 mL), filtered through a pad of Celite, and the pad washed with EtOAc and the filtrate dried under reduced pressure. The crude material obtained was purified by normal phase chromatography using 0-50% EtOAc in n-hexane to afford intermediate 55 which was used directly in the next step.

### Step-2: 4-(2-methylsulfonyl-phenyl)-oxazole-5-carboxylic acid (56)

To a solution of intermediate ester (55, 290 mg, 0.982 mmol, 1.0 eq) in a mixture of THF (4.6 mL) was added a solution of LiOH·H₂O (165 mg, 3.93 mmol, 4.0 eq) in water (4.6 mL) at rt. The reaction mixture was stirred at RT for 16 h. After completion of the reaction the solution was cooled to 0° C. and acidified with aq. HCl (pH-2) and extracted with EtOAc. The combined organic layer was dried over Na₂SO₄ and concentrated to afford 56. The crude compound was used directly in the next step.

### Step-3: Example 44

In line with the General Procedure A, acid 56 (255 mg, 1 eq) was coupled with 4-(pyridin-2-yl)thiazol-2-amine (4) (186 mg, 1.1 eq) to afford Example 44. ¹H NMR (400 MHz, DMSO-d₆) δ 8.86 (s, 1H), 8.60 (ddd, J=4.8, 1.8, 1.0 Hz, 1H), 8.13-8.06 (m, 1H), 8.01 (dt, J=7.9, 1.1 Hz, 1H), 7.94-7.75 (m, 4H), 7.70-7.64 (m, 1H), 7.34 (ddd, J=7.6, 4.8, 1.3 Hz, 1H), CH3 hidden under water peak, NH not observed; LC-MS: m/z 427.0 [M+H]⁺.

### Synthesis of Example 45

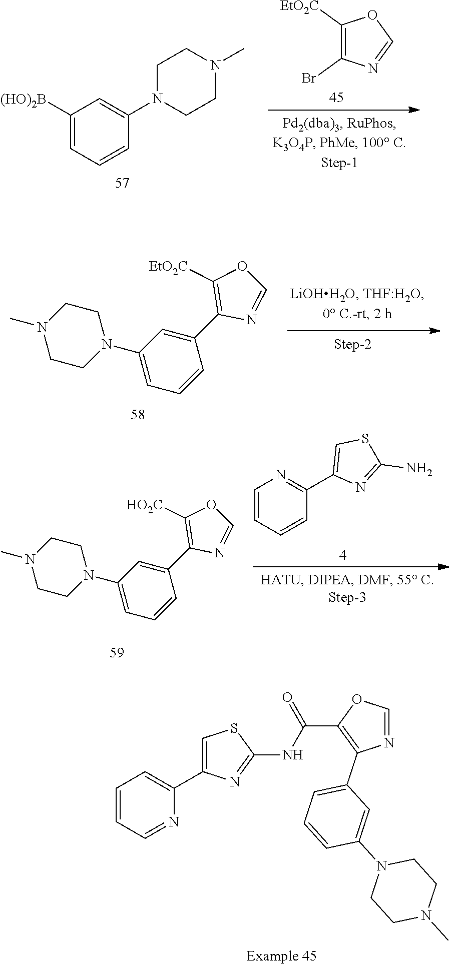

### Step-1: Ethyl 4-(3-(4-methyl-piperazin-1-yl)-phenyl)-oxazole-5-carboxylate (58)

In a microwave vial equipped with a magnetic stir bar was charged with ethyl 4-bromo-1,3-oxazole-5-carboxylate (45) (400 mg, 1.82 mmol, 1.0 eq), Pd₂(dba)₃ (66.6 mg, 0.04 eq), RuPhos (67.8 mg, 0.08 eq) and 1-(3-boronic acid-phenyl)-4-methyl-piperazine (57, 480 mg, 2.18 mmol, 1.2 eqv) anhydrous crushed K₃PO₄ (1.26 g, 3.0 eq). Dry toluene (7.3 mL) was added, and the vial capped with a rubber septum and purged with argon for 10 min. The reaction mixture was heated in an oil bath at 100° C. with vigorous stirring for 18 h. The reaction mixture was then allowed to cool to room temperature, diluted with EtOAc (50 mL), filtered through a pad of Celite, and the pad washed with EtOAc and the filtrate dried under reduced pressure. The crude material obtained was purified by normal phase chromatography using 0-50% EtOAc in n-hexane to afford intermediate 58 which was used directly in the next step.

### Step-2: 4-(3-(4-methyl-piperazin-1-yl)-phenyl)-oxazole-5-carboxylic acid (59)

To a solution of intermediate ester (58, 400 mg, 1.0 eqv) in a mixture of THF (6.3 mL) was added MeOH (0.29 mL) and a solution of LiOH·H₂O (213 mg, 4.0 eq) in water (6 mL) at rt.

The reaction mixture was stirred at RT for 16 h. After completion of the reaction the solution was cooled to 0° C. and acidified with aq. HCl (pH-6) and extracted with EtOAc. The combined organic layer was dried over Na₂SO₄ and concentrated to afford 59. The crude compound was used directly in the next step.

### Step-3: Example 45

In line with the General Procedure A, acid 59 (150 mg, 1 eq) was coupled with 4-(pyridin-2-yl)thiazol-2-amine (4) (177 mg, 1.05 eq) to afford Example 45. ¹H NMR (400 MHz, DMSO-d₆) δ 8.77 (s, 1H), 8.65-8.58 (m, 1H), 8.04-7.97 (m, 1H), 7.96-7.86 (m, 2H), 7.77 (d, J=2.2 Hz, 1H), 7.61 (d, J=7.8 Hz, 1H), 7.42-7.31 (m, 2H), 7.14-7.07 (m, 1H), 11 aliphatic H's hidden under solvent peaks, NH not observed; LC-MS: m/z 447.2 [M+H]⁺.

### Synthesis of Example 46

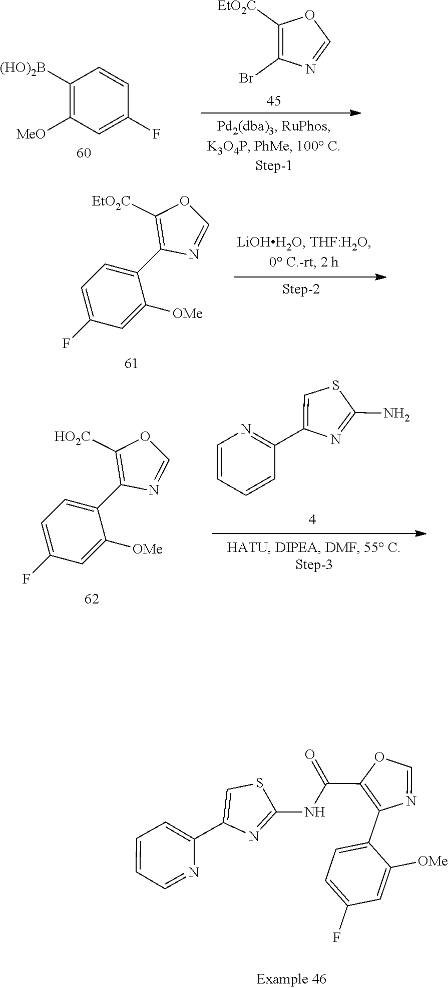 [SMILES: COc1cc(F)ccc1B(O)O ; CCOC(=O)c1ocnc1Br ; CCOC(=O)c1ocnc1-c1ccc(F)cc1OC ; COc1cc(F)ccc1-c1ncoc1C(=O)O ; Nc1nc(-c2ccccn2)cs1 ; COc1cc(F)ccc1-c1ncoc1C(=O)Nc1nc(-c2ccccn2)cs1]

### Step-1: Ethyl 4-(4-fluoro-2-methoxy-phenyl)-oxazole-5-carboxylate (61)

In a microwave vial equipped with a magnetic stir bar was charged with ethyl 4-bromo-1,3-oxazole-5-carboxylate (45) (200 mg, 0.91 mmol, 1.0 eq), Pd₂(dba)₃ (33.3 mg, 0.04 eq), RuPhos (33.9 mg, 0.08 eq) and 4-fluoro-2-methoxy-phenyl boronic acid (60, 170 mg, 1.09 mmol, 1.2 eq) anhydrous crushed K₃PO₄ (579 mg, 3.0 eq). Dry toluene (2 mL) was added, and the vial capped with a rubber septum and purged with argon for 10 min. The reaction mixture was heated in an oil bath at 100° C. with vigorous stirring for 18 h. The reaction mixture was then allowed to cool to room temperature, diluted with EtOAc (50 mL), filtered through a pad of Celite, and the pad washed with EtOAc and the filtrate dried under reduced pressure. The crude material obtained was purified by normal phase chromatography using 0-50% EtOAc in n-hexane to afford intermediate 61 (210 mg, 87%) which was used directly in the next step.

### Step-2: 4-(5-fluoro-2-methylsulfonyl-phenyl)-oxazole-5-carboxylic acid (62)

To a solution of intermediate ester (61, 210 mg, 1.0 eqv) in a mixture of THF (4.1 mL) was added a solution of LiOH·H₂O (133 mg, 4.0 eq) in water (4.1 mL) at rt. The reaction mixture was stirred at RT for 16 h. After completion of the reaction the solution was cooled to 0° C. and acidified with aq. HCl (pH-2) and extracted with EtOAc. The combined organic layer was dried over Na₂SO₄ and concentrated to afford 62 (172 mg, 92%). The crude compound was used directly in the next step.

### Step-3: Example 46

In line with the General Procedure A, acid 62 (172 mg, 1 eq) was coupled with 4-(pyridin-2-yl)thiazol-2-amine (4) (142 mg, 1.1 eq) to afford Example 46 (143 mg, 50%). ¹H NMR (400 MHz, DMSO-d₆) δ 8.70 (s, 1H), 8.60 (ddd, J=4.8, 1.8, 0.9 Hz, 1H), 7.96 (dt, J=8.0, 1.2 Hz, 1H), 7.90 (s, 1H), 7.92-7.84 (m, 1H), 7.59 (dd, J=8.5, 6.9 Hz, 1H), 7.34 (ddd, J=7.5, 4.8, 1.3 Hz, 1H), 7.03 (dd, J=11.4, 2.5 Hz, 1H), 6.92 (td, J=8.4, 2.5 Hz, 1H), 3.63 (s, 3H), NH not observed; LC-MS: m/z 397.1 [M+H]⁺.

### Synthesis of Example 47

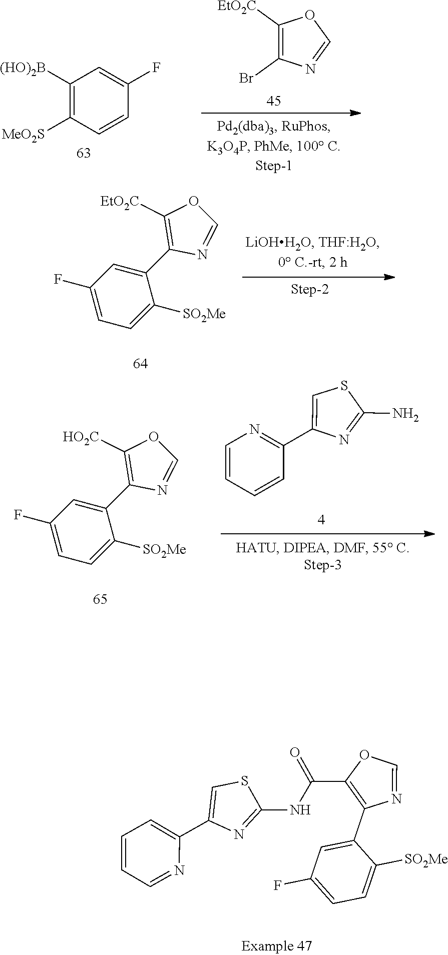 [SMILES: CS(=O)(=O)c1ccc(F)cc1B(O)O ; CCOC(=O)c1ocnc1Br ; CCOC(=O)c1ocnc1-c1cc(F)ccc1S(C)(=O)=O ; CS(=O)(=O)c1ccc(F)cc1-c1ncoc1C(=O)O ; Nc1nc(-c2ccccn2)cs1 ; CS(=O)(=O)c1ccc(F)cc1-c1ncoc1C(=O)Nc1nc(-c2ccccn2)cs1]

### Step-1: Ethyl 4-(5-fluoro-2-methylsulfonyl-phenyl)-oxazole-5-carboxylate (64)

In a microwave vial equipped with a magnetic stir bar was charged with ethyl 4-bromo-1,3-oxazole-5-carboxylate (45) (250 mg, 1.136 mmol, 1.0 eq), Pd₂(dba)₃ (41.6 mg, 0.04 eq), RuPhos (42.4 mg, 0.08 eq) and 5-fluoro-2-methylsulfonyl-phenyl boronic acid (63, 297 mg, 1.364 mmol, 1.2 eq) anhydrous crushed K₃PO₄ (724 mg, 3.0 eqv). Dry toluene (2.5 mL) was added, and the vial capped with a rubber septum and purged with argon for 10 min. The reaction mixture was heated in an oil bath at 100° C. with vigorous stirring for 1.5 h. The reaction mixture was then allowed to cool to room temperature, diluted with EtOAc (50 mL), filtered through a pad of Celite, and the pad washed with EtOAc and the filtrate dried under reduced pressure. The crude material obtained was purified by normal phase chromatography using 0-50% EtOAc in n-hexane to afford intermediate 64 (200 mg, 56%) which was used directly in the next step.

### Step-2: 4-(4-fluoro-2-methoxy-phenyl)-oxazole-5-carboxylic acid (65)

To a solution of intermediate ester (64, 200 mg, 1.0 eqv) in a solution of THF (3 mL) was added a solution of LiOH·H₂O (107 mg, 4.0 eq) in water (3 mL) at rt. The reaction mixture was stirred at RT for 16 h. After completion of the reaction the solution was cooled to 0° C. and acidified with aq. HCl (pH-2) and extracted with EtOAc. The combined organic layer was dried over Na₂SO₄ and concentrated to afford 65 (169 mg, 93%). The crude compound was used directly in the next step.

### Step-3: Example 47

In line with the General Procedure A, acid 65 (169 mg, 1 eq) was coupled with 4-(pyridin-2-yl)thiazol-2-amine (4) (116 mg, 1.1 eq) to afford Example 47 (100 mg, 38%). ¹H NMR (400 MHz, DMSO-d₆) δ 8.88 (s, 1H), 8.61 (ddd, J=4.8, 1.8, 0.9 Hz, 1H), 8.16 (dd, J=9.6, 5.6 Hz, 1H), 8.01 (dt, J=7.9, 1.1 Hz, 1H), 7.90 (td, J=7.7, 1.8 Hz, 1H), 7.89 (s, 1H), 7.71-7.61 (m, 2H), 7.34 (ddd, J=7.5, 4.8, 1.2 Hz, 1H), 3.3.3 (s, 3H), NH not observed; LC-MS: m/z 445.0 [M+H]⁺.

### Synthesis of Example 48

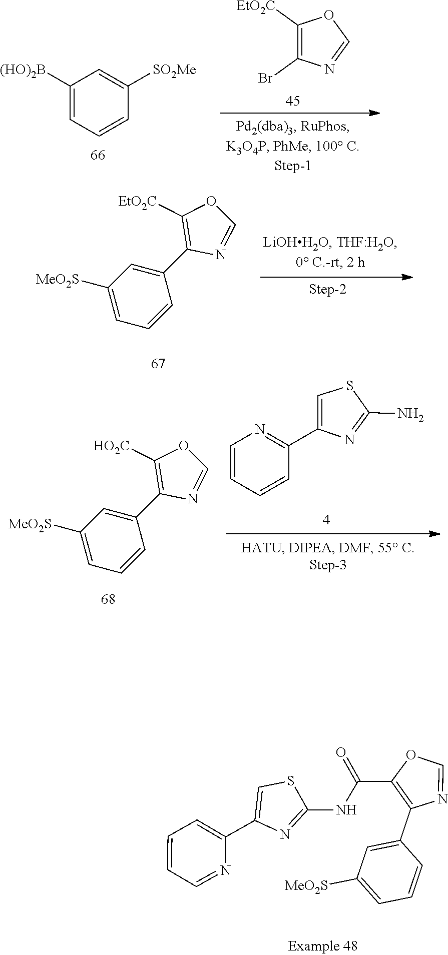

### Step-1: Ethyl 4-(3-methylsulfonyl-phenyl)-oxazole-5-carboxylate (67)

In a microwave vial equipped with a magnetic stir bar was charged with ethyl 4-bromo-1,3-oxazole-5-carboxylate (45) (300 mg, 1.363 mmol, 1.0 eq), Pd₂(dba)₃ (50 mg, 0.04 eq), RuPhos (50.8 mg, 0.08 eq) and 3-methylsulfonyl-phenyl boronic acid (66, 327 mg, 1.637 mmol, 1.2 eq) anhydrous crushed K₃PO₄ (868 mg, 3.0 eq). Dry toluene (3 mL) was added, and the vial capped with a rubber septum and purged with argon for 10 min. The reaction mixture was heated in an oil bath at 100° C. with vigorous stirring for 1.5 h. The reaction mixture was then allowed to cool to room temperature, diluted with EtOAc (50 mL), filtered through a pad of Celite, and the pad washed with EtOAc and the filtrate dried under reduced pressure. The crude material obtained was purified by normal phase chromatography using 0-50% EtOAc in n-hexane to afford intermediate 67 (240 mg, 60%) which was used directly in the next step.

### Step-2: 4-(3-methylsulfonyl-phenyl)-oxazole-5-carboxylic acid (68)

To a solution of intermediate ester (67, 240 mg, 1.0 eqv) in a solution of THF (3.8 mL) was added a solution of LiOH·H₂O (136 mg, 4.0 eq) in water (3 mL) at rt. The reaction mixture was stirred at RT for 16 h. After completion of the reaction the solution was cooled to 0° C. and acidified with aq. HCl (pH-2) and extracted with EtOAc. The combined organic layer was dried over Na₂SO₄ and concentrated to afford 68 (186 mg). The crude compound was used directly in the next step.

### Step-3: Example 48

In line with the General Procedure A, acid 68 (186 mg, 1 eq) was coupled with 4-(pyridin-2-yl)thiazol-2-amine (4) (136 mg, 1.1 eq) to afford Example 48 (200 mg, 67%). ¹H NMR (400 MHz, DMSO-d₆) δ 8.87 (s, 1H), 8.69-8.58 (m, 2H), 8.52 (dt, J=7.9, 1.4 Hz, 1H), 8.02 (ddt, J=8.1, 7.2, 1.0 Hz, 2H), 7.94 (s, 1H), 7.90 (td, J=7.7, 1.8 Hz, 1H), 7.82 (t, J=7.8 Hz, 1H), 7.35 (ddd, J=7.5, 4.8, 1.2 Hz, 1H), 3.27 (s, 3H), NH not observed; LC-MS: m/z 427.0 [M+H]⁺.

### Synthesis of Example 49

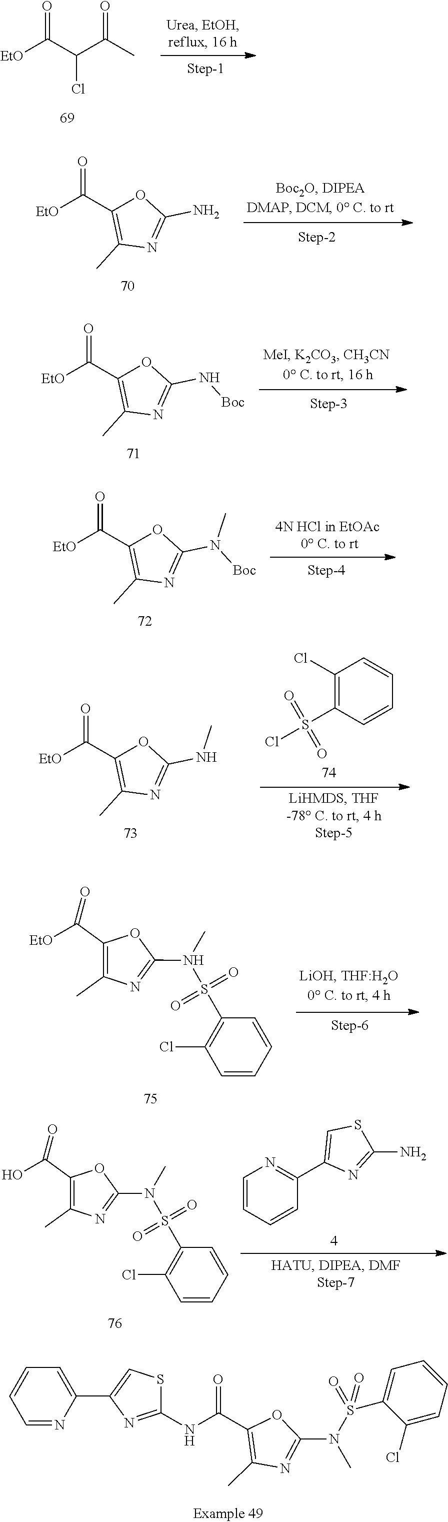

### Step-1: Ethyl 2-amino-4-methyloxazole-5-carboxylate (70)

A suspension of ethyl 2-chloro-3-oxobutanoate (69) (7.0 g, 42.53 mmol, 1 eq) and urea (7.66 g, 20.57 mmol, 3 eq) in EtOH (90 mL) was refluxed for 16 h. Progress of reaction was monitored by TLC. After completion, the reaction mixture was concentrated, and crude obtained was washed with H₂O (2×50 mL) and triturated with Et₂O (2×50 mL) to afford 70 (3.50 g, 48%). ¹H NMR (400 MHz, DMSO-d6) δ=7.42 (s, 2H), 4.18 (q, J=7.0 Hz, 2H), 2.22 (s, 3H), 1.24 (t, J=7.1 Hz, 3H); LC-MS: m/z 170.8 [M+H]⁺.

### Step-2: Ethyl 2-((tert-butoxycarbonyl)amino)-4-methyloxazole-5-carboxylate (71)

To a solution of ethyl 2-amino-4-methyloxazole-5-carboxylate (70) (1.0 g, 5.88 mmol, 1 eq) in DCM (15 mL) maintained at 0° C. was added DIPEA (2.5 mL, 17.63 mmol, 3 eq), DMAP (72 mg, 0.58 mmol, 0.1 eq) followed by Boc₂O (2.0 mL, 8.81 mmol, 1.5 eq). Reaction mixture was stirred at rt for 16 h. Progress of reaction was monitored by TLC. After completion, the reaction mixture was concentrated, and crude obtained was dissolved in EtOAc (100 mL).

The organic layer was washed with H₂O (2×100 mL), dried over anhydrous Na₂S₂O₄ and concentrated. The crude obtained was purified by column purification (silica 100-200 mesh; 18% EtOAc in Hexanes) to afford (71) (500 mg, 31%). LC-MS: m/z 214.9 [M-tBu]⁺

### Step-3: Ethyl 2-((tert-butoxycarbonyl)(methyl)amino)-4-methyloxazole-5-carboxylate (72)

To a stirred solution of ethyl 2-((tert-butoxycarbonyl)amino)-4-methyloxazole-5-carboxylate (71) (500 mg, 1.85 mmol, 1 eq) and K₂CO₃ (640 mg, 4.62 mmol, 2.5 eq) in CH₃CN (5 mL) maintained at 0° C. was added Mel (790 mg, 5.55 mmol, 3 eq). Reaction mixture was stirred at rt for 16 h. The progress of the reaction was monitored by TLC. After completion, the reaction mixture was quenched H₂O (10 mL) and extracted with EtOAc (2×10 mL). The combined organic layer was washed with brine, dried over anhydrous Na₂S₂O₄ and concentrated under reduced pressure. The crude obtained was purified by solvent trituration using Et₂O (10 mL) to afford 72 (510 mg). This was used as such for the next reaction. ¹H NMR (400 MHz, DMSO-d6) δ=4.26 (q, J=7.3 Hz, 2H), 3.28 (s, 3H), 2.35 (s, 3H), 1.49 (s, 9H), 1.28 (t, J=7.1 Hz, 3H); LC-MS: m/z 228.9 [M-^{t}Bu]⁺.

### Step-4: Ethyl 4-methyl-2-(methylamino)oxazole-5-carboxylate (73)

To a solution of ethyl 2-((tert-butoxycarbonyl)(methyl)amino)-4-methyloxazole-5-carboxylate (72) (500 mg, 1.76 mmol, 1 eq) in EtOAc (5 mL) maintained at 0° C. was added 4N HCl in EtOAc (2.5 mL). Reaction was stirred at rt for 16 h. Progress of reaction was monitored by TLC. After completion, the reaction mixture was concentrated, and crude obtained was washed with Et₂O (10 mL) to afford 73 (480 mg as HCl salt). This was used as such for the next reaction. ¹H NMR (400 MHz, DMSO-d6) δ=4.18 (q, J=6.8 Hz, 2H), 2.80 (s, 3H), 2.25 (s, 3H), 1.22 (t, J=7.1 Hz, 3H)[NH was not observed]; LC-MS: m/z 184.9 [M+H]⁺.

### Step-5: Ethyl 2-((2-chloro-N-methylphenyl)sulfonamido)-4-(trifluoromethyl)oxazole-5-carboxylate (75)

To a solution of ethyl 4-methyl-2-(methylamino)oxazole-5-carboxylate (73) (450 mg, 2.44 mmol, 1 eq) in THE (5 mL) maintained at −78° C. was added LiHMDS (1.5 mL, 6.11 mmol, 2.5 eq). Reaction mixture was stirred at same temperature for 10 min. To this was added 2-chlorobenzenesulfonyl chloride (74) (774 mg, 3.66 mmol, 1.5 eq) and the reaction was allowed to warm at rt. The reaction mixture was stirred at ambient temperature for 4 h. Progress of reaction was monitored by TLC and LCMS. After completion, reaction mixture was quenched with H₂O (10 mL) and diluted with EtOAc (50 mL). Organic layer was washed with H₂O (2×20 mL) and brine solution (2×20 mL), dried over anhydrous Na₂S₂O₄ and concentrated. The crude obtained was purified by column chromatography (silica 100-200 mesh; 40% EtOAc in Hexanes) to afford 75 (500 mg, 50%). ¹H NMR (400 MHz, DMSO-d₆) δ=8.21 (d, J=8.3 Hz, 1H), 7.78-7.68 (m, 2H), 7.67-7.60 (m, 1H), 4.18 (q, J=7.3 Hz, 2H), 3.52 (s, 3H), 2.22 (s, 3H).

### Step-6: 2-((2-chloro-N-methylphenyl)sulfonamido)-4-(trifluoromethyl)oxazole-5-carboxylic acid (76)

To a solution of ethyl 2-((2-chloro-N-methylphenyl)sulfonamido)-4-(trifluoromethyl)oxazole-5-carboxylate (75) (500 mg, 1.21 mmol. 1 eq) in THF:H₂O (4:1; 8 mL) maintained at 0° C. was added LiGH (73 mg, 3.03 mmol, 2.5 eq). Reaction mixture was stirred at rt for 12 h. Progress of reaction was monitored by TLC. After completion, reaction mixture was concentrated and washed with Et₂O. The crude obtained was acidified with 2N HCl to afford 76 (350 mg). This was triturated using Et₂O and used for the next reaction. ¹H NMR (400 MHz, DMSO-d₆) δ=13.37-13.15 (m, 1H), 8.21 (d, J=7.8 Hz, 1H), 7.80-7.72 (m, 2H), 7.70-7.57 (m, 1H), 3.53 (s, 3H).

### Step-7: Example 49

In line with the General Procedure A, acid 76 was coupled with 4-(pyridin-2-yl)thiazol-2-amine (4) to afford Example 49. ¹H NMR (400 MHz, DMSO-d₆) δ 12.66 (s, 1H), 8.62 (bs, 1H), 8.27 (d, J=8.0 Hz, 1H), 8.02 (d, J=8.0 Hz, 1H), 7.91-7.88 (m, 2H), 7.79-7.73 (m, 2H), 7.68 (t, J=7.6 Hz, 1H), 7.36 (t, J=6.4 Hz, 1H), 3.26 (s, 3H), 2.27 (s, 3H); LC-MS: m/z 490.0 [M+H]⁺.

### Synthesis of Example 50

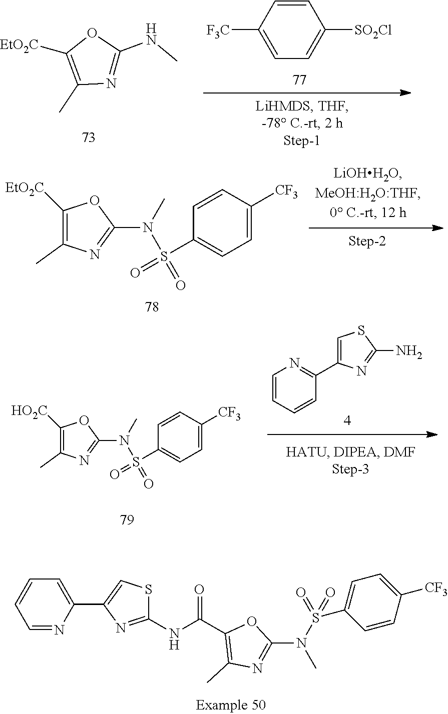 [SMILES: CCOC(=O)c1oc(NC)nc1C ; O=S(=O)(Cl)c1ccc(C(F)(F)F)cc1 ; CCOC(=O)c1oc(N(C)S(=O)(=O)c2ccc(C(F)(F)F)cc2)nc1C ; Cc1nc(N(C)S(=O)(=O)c2ccc(C(F)(F)F)cc2)oc1C(=O)O ; Nc1nc(-c2ccccn2)cs1 ; Cc1nc(N(C)S(=O)(=O)c2ccc(C(F)(F)F)cc2)oc1C(=O)Nc1nc(-c2ccccn2)cs1]

### Step-1: Ethyl 4-methyl-2-((N-methyl-4-(trifluoromethyl)phenyl)sulfonamido)oxazole-5-carboxylate (78)

To a stirred solution of ethyl 4-methyl-2-(methylamino)oxazole-5-carboxylate (73, 150 mg, 0.82 mmol, 1.0 eq) in dry THE (25 mL) was added LiHMDS (1M in THF) (11.2 mL, 1.23 5 mmol, 1.5 eq) (1M in THF) at 0° C. After stirring for 10 min, a solution of 4-(trifluoromethyl)benzenesulfonyl chloride (77, 240 mg, 0.98 mmol, 1.2 eq) was dissolved into THF (5 mL) was added dropwise over 10 min. Then, slowly warmed at RT and stirred for 2 h.

The progress of the reaction was monitored by TLC. After completion of reaction, the reaction mixture was quenched with saturated NH₄Cl at 0° C. and extracted with ethyl acetate. The combined organic layer was dried over anhydrous Na₂SO₄ and concentrated under reduced pressure. The crude compound was purified by Silica gel (100-200) column chromatography (5-10% EtOAc/hexane) to afford ethyl 4-methyl-2-((N-methyl-4-(trifluoromethyl)phenyl)sulfonamido)oxazole-5-carboxylate (78, 300 mg, 94%) as a white solid. LC-MS: m/z 392.90 [M+H]⁺.

### Step-2: 4-Methyl-2-((N-methyl-4 (trifluoromethyl)phenyl)sulfonamido)oxazole-5-carboxylic acid (79)

To a stirred solution of ethyl 4-methyl-2-((N-methyl-4-(trifluoromethyl)phenyl)sulfonamido)oxazole-5-carboxylate (78, 300 mg, 0.76 mmol, 1.0 eq) in a mixture of THF:MeO:H₂O (2:1:0.5) was added LiOH·H₂O (64.30 mg, 1.53 mmol, 2.0 eq) at 0° C. The RM was stirred at RT for 2 h. After the completion of the reaction, the RM was concentrated. The residue was diluted with water and acidified with 6N HCl at 0° C. The white precipitate was extracted with 5% MeOH in DCM. The organic layer was dried and concentrated. The crude compound was washed with 10% ether and n-Hexane to afford The crude compound was purified through prep-HPLC to afford 4-methyl-2-((N-methyl-4-(trifluoromethyl)phenyl)sulfonamido)oxazole-5-carboxylic acid (79, 278 mg, 65%) as a white solid. ¹H NMR (400 MHz, DMSO-d₆): δ 13.45 (br s, 1H), 8.21 (d, J=8.40 Hz, 1H), 8.06 (d, J=8.0 Hz, 1H), 7.98 (d, J=7.6 Hz, 1H), 7.79-7.70 (m, 1H), 3.43 (s, 3H), 2.87 (s, 3H); LC-MS: m/z 364.85 [M+H]⁺.

### Step-3: Example 50

In line with the General Procedure A, acid 79 was coupled with 4-(pyridin-2-yl)thiazol-2-amine (4) to afford Example 50. ¹H NMR (400 MHz, DMSO-d₆) δ 12.71 (s, 1H), 8.60 (d, J=4.4 Hz, 1H), 8.30 (d, J=7.6 Hz, 2H), 8.08 (d, J=8.4 Hz, 2H), 8.00 (d, J=8.4 Hz, 1H), 7.89 (t, J=6.4 Hz, 2H), 7.34 (t, J=6.0 Hz, 1H), 3.62 (s, 3H), 2.39 (s, 3H); LC-MS: m/z 524.0 [M+H]⁺; HPLC: 96.8%.

### Synthesis of Example 51

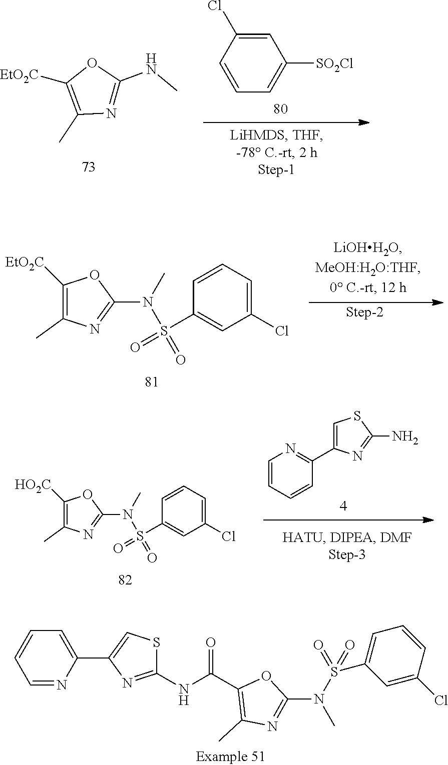 [SMILES: CCOC(=O)c1oc(NC)nc1C ; O=S(=O)(Cl)c1cccc(Cl)c1 ; CCOC(=O)c1oc(N(C)S(=O)(=O)c2cccc(Cl)c2)nc1C ; Cc1nc(N(C)S(=O)(=O)c2cccc(Cl)c2)oc1C(=O)O ; Nc1nc(-c2ccccn2)cs1 ; Cc1nc(N(C)S(=O)(=O)c2cccc(Cl)c2)oc1C(=O)Nc1nc(-c2ccccn2)cs1]

### Step-1: Ethyl 2-((3-chloro-N-methylphenyl)sulfonamido)-4-methyloxazole-5-carboxylate (81)

To a stirred mixture of ethyl 4-methyl-2-(methylamino)oxazole-5-carboxylate (73) (500 mg, 2.71 mmol, 1 eq) in THF (50 mL), LiHMDS (1M in THF, 6.7 mL, 6.79 mmol, 2.5 eq) was added at 78° C. and the mixture was stirred for 30 min at same temperature. To the resulting mixture, 3-chlorobenzenesulfonyl chloride (80) (0.86 g, 4.07 mmol, 1.5 eq) was added into the reaction mixture and allowed to stirred at RT for 12 h. The progress of the reaction was monitored by TLC (M.Ph: 30% EtOAc in n-hexane). The reaction mixture diluted with water (75 mL) and extracted with EtOAc (2×150 mL). The combined organic layers was washed with brine, dried over sodium sulfate, filtered and concentrated to dryness. The crude was purified through silica gel column chromatography (elution: 15% EtOAc in n-hexane) to afford 81 (0.4 g, 41%) as an off-white solid. ¹H NMR (400 MHz, DMSO-d₆) δ 8.06 (s, 1H), 7.98 (d, J=8.0 Hz, 1H), 7.89 (d, J=7.6 Hz, 1H), 7.73 (t, J=8.0 Hz, 1H), 4.31 (q, J=6.8 Hz, 2H), 3.45 (s, 3H), 2.30 (s, 3H), 1.32 (t, J=7.2 Hz, 3H); LC-MS: m/z 358.9 [M+H]⁺.

### Step-2: 2-((3-Chloro-N-methylphenyl)sulfonamido)-4-methyloxazole-5-carboxylic acid (82)

To a stirred solution of ethyl 2-((3-chloro-N-methylphenyl)sulfonamido)-4-methyloxazole-5-carboxylate (81) (400 mg, 1.11 mmol, 1 eq) in THE (5 mL), MeOH (5 mL), H₂O (2 mL), LiOH·H₂O (137 mg, 2.79 mmol, 2.5 eq) was added at 0° C. and the reaction mixture was stirred at RT for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 82 (250 mg, 68%) as an off-white solid. ¹H NMR (400 MHz, DMSO-d₆) δ 13.33 (s, 1H), 8.05 (s, 1H), 7.96 (d, J=8.0 Hz, 1H), 7.88 (d, J=7.6 Hz, 1H), 7.72 (t, J=8.0 Hz, 1H), 3.37 (s, 3H), 2.28 (s, 3H); LC-MS: m/z 331.1 [M+H]⁺.

### Step-3: Example 51

In line with the General procedure A, 2-((4-chloro-N-methylphenyl)sulfonamido)-4-methyloxazole-5-carboxylic acid (82) (250 mg, 0.75 mmol, 1 eq) was coupled with 4-(pyridin-2-yl)thiazol-2-amine (4) (147 mg, 0.99 mmol, 1.1 eq) to afford Example 51 (70 mg, 19%) as an off-white solid. ¹H NMR (400 MHz, DMSO-d₆) δ 12.70 (s, 1H), 8.61 (d, J=4.4 Hz, 1H), 8.15 (s, 1H), 8.05-8.00 (m, 2H), 7.90-7.87 (m, 3H), 7.75-7.70 (m, 1H), 7.36 (t, J=8.4 Hz, 1H), 3.61 (s, 3H), 2.39 (s, 3H); LC-MS: m/z 490.00 [M+H]⁺; HPLC: 99.7%.

### Synthesis of Example 52

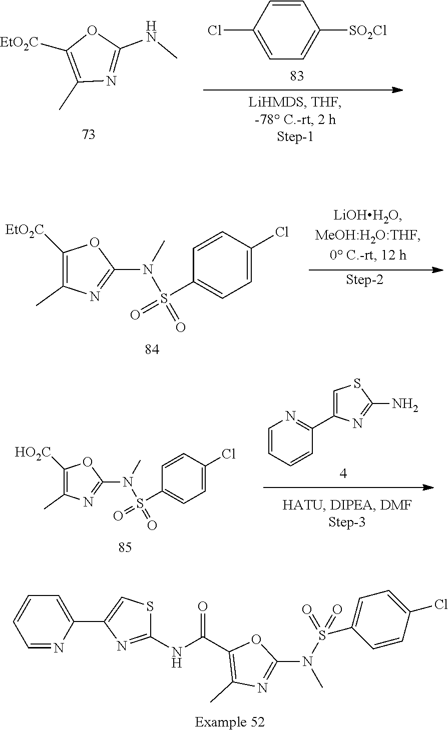

### Step-1: Ethyl 2-((4-chloro-N-methylphenyl)sulfonamido)-4-methyloxazole-5-carboxylate (84)

To a stirred mixture of ethyl 4-methyl-2-(methylamino)oxazole-5-carboxylate (73) (500 mg, 2.71 mmol, 1 eq) in THF (50 mL), LiHMDS (1M in THF, 6.7 mL, 6.79 mmol, 2.5 eq) was added at 78° C. and the mixture was stirred for 30 min at same temperature. To the resulting mixture, 4-chlorobenzenesulfonyl chloride (83) (0.86 g, 4.07 mmol, 1.5 eq) was added into the reaction mixture and allowed to stirred at RT for 12 h. The progress of the reaction was monitored by TLC (M.Ph: 30% EtOAc in n-hexane). The reaction mixture diluted with water (75 mL) and extracted with EtOAc (2×150 mL). The combined organic layers was washed with brine, dried over sodium sulfate, filtered and concentrated to dryness. The crude was purified through silica gel column chromatography (elution: 15% EtOAc in n-hexane) to afford 84 (0.8 g, 82%) as an off-white solid. ¹H NMR (400 MHz, DMSO-d₆) δ 8.01 (d, J=8.4 Hz, 2H), 7.76 (d, J=8.4 Hz, 2H), 4.30 (q, J=6.8 Hz, 2H), 3.41 (s, 3H), 2.30 (s, 3H), 1.31 (t, J=7.2 Hz, 3H); LC-MS: m/z 358.9 [M+H]⁺.

### Step-2: 2-((4-Chloro-N-methylphenyl)sulfonamido)-4-methyloxazole-5-carboxylic acid (85)

To a stirred solution of ethyl 2-((4-chloro-N-methylphenyl)sulfonamido)-4-methyloxazole-5-carboxylate (84) (560 mg, 1.39 mmol, 1 eq) in THE (5 mL), MeOH (5 mL), H₂O (2 mL), LiOH·H₂O (146 mg, 3.49 mmol, 2.5 eq) was added at 0° C. and the reaction mixture was stirred at RT for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 85 (408 mg, 89%) as an off-white solid. ¹H NMR (400 MHz, DMSO-d₆) δ 8.00 (d, J=6.8 Hz, 2H), 7.76 (d, J=8.48 Hz, 2H), 3.39 (s, 3H), 2.28 (s, 3H); LC-MS: m/z 331.1 [M+H]⁺.

### Step-3: Example 52

In line with the General procedure A, 2-((4-chloro-N-methylphenyl)sulfonamido)-4-methyloxazole-5-carboxylic acid (85) (300 mg, 0.90 mmol, 1 eq) was coupled with 4-(pyridin-2-yl)thiazol-2-amine (4) (176 mg, 0.99 mmol, 1.1 eq) to afford Example 52 (30 mg, 7%) as an off-white solid. ¹H NMR (400 MHz, DMSO-d₆) δ 12.87 (s, 1H), 8.61 (d, J=4.4 Hz, 1H), 8.08 (d, J=8.8 Hz, 2H), 8.01 (d, J=8.0 Hz, 1H), 7.92-7.86 (m, 2H), 7.77 (d, J=8.8 Hz, 2H), 7.36-7.32 (m, 1H), 3.59 (s, 3H), 2.39 (s, 3H); LC-MS: m/z 490.00 [M+H]⁺; HPLC: 99.7%.

### Synthesis of Example 53

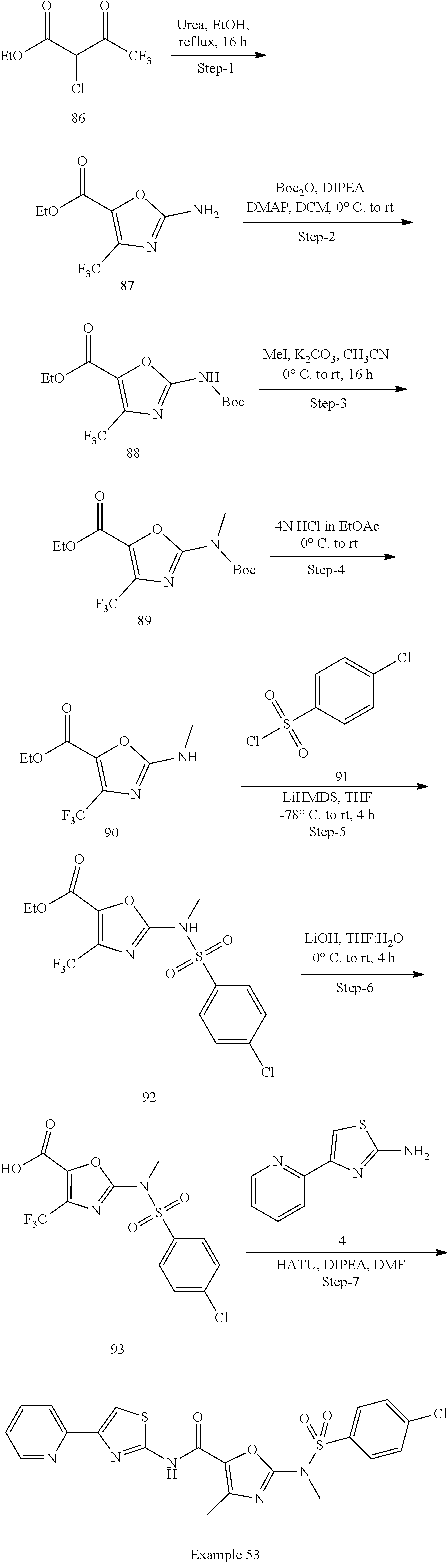

### Step-1: Ethyl 2-amino-4-(trifluoromethyl)oxazole-5-carboxylate (87)

A suspension of ethyl 2-chloro-4,4,4-trifluoro-3-oxobutanoate (86) (5.0 g, 22.88 mmol, 1 eq) and urea (4.12 g, 68.63 mmol, 3 eq) in EtOH (80 mL) was refluxed for 16 h. Progress of reaction was monitored by TLC. After completion, the reaction mixture was concentrated, and crude obtained was washed with H₂O (2×50 mL) and triturated with Et₂O (2×50 mL) to afford (87) (2.20 g, 42%). ¹H NMR (400 MHz, DMSO-d6) δ 8.01 (s, 2H), 4.26 (q, J=6.8 Hz, 2H), 1.26 (t, J=7.1 Hz, 3H); LC-MS: m/z 224.9 [M+H]⁺.

### Step-2: Ethyl 2-((tert-butoxycarbonyl)amino)-4-(trifluoromethyl)oxazole-5-carboxylate (88)

To a solution of ethyl 2-amino-4-(trifluoromethyl)oxazole-5-carboxylate (87) (4.4 g, 19.63 mmol, 1 eq) in DCM (60 mL) maintained at 0° C. was added DIPEA (8.0 mL, 58.89 mmol, 3 eq), DMAP (240 mg, 1.96 mmol, 0.1 eq) followed by Boc₂O (6.43 g, 29.45 mmol, 1.5 eq).

Reaction mixture was stirred at rt for 16 h. Progress of reaction was monitored by TLC. After completion, the reaction mixture was concentrated, and crude obtained was dissolved in EtOAc (100 mL). The organic layer was washed with H₂O (2×100 mL), dried over anhydrous Na₂S₂O₄ and concentrated. The crude obtained was purified by column purification (silica 100-200 mesh; 25% EtOAc in Hexanes) to afford (88) (4.50 g, 71%). ¹H NMR (400 MHz, DMSO-d₆) δ=11.66-11.13 (m, 1H), 4.32 (q, J=7.3 Hz, 2H), 1.46 (s, 9H), 1.28 (t, J=7.1 Hz, 3H) [NH was not observed]; LC-MS: m/z 268.9 [M-tBu]⁺.

### Step-3: Ethyl 2-((tert-butoxycarbonyl)(methyl)amino)-4-(trifluoromethyl)oxazole-5-carboxylate (90)

To a stirred solution of ethyl 2-((tert-butoxycarbonyl)amino)-4-(trifluoromethyl)oxazole-5-carboxylate (88) (4.30 g, 13.26 mmol, 1 eq) and K₂CO₃ (4.58 g, 33.15 mmol, 2.50 eq) in CH₃CN (60 mL) maintained at 0° C. was added Mel (5.65 g, 39.78 mmol, 3 eq). Reaction mixture was stirred at rt for 16 h. The progress of the reaction was monitored by TLC. After completion, the reaction mixture was quenched H₂O (100 mL) and extracted with EtOAc (2×100 mL). The combined organic layer was washed with brine, dried over anhydrous Na₂S₂O₄ and concentrated under reduced pressure. The crude obtained was purified by column chromatography (silica-100-200 mesh; 30% EtOAc in Hexane) to afford 89 (3.60 g, 80%). LC-MS: m/z 282.9 [M-tBu]⁺.

### Step-4: Ethyl 2-(methylamino)-4-(trifluoromethyl)oxazole-5-carboxylate (90)

To a solution of ethyl 2-((tert-butoxycarbonyl)(methyl)amino)-4-(trifluoromethyl)oxazole-5-carboxylate (89) (3.60 g, 10.64 mmol, 1 eq) in EtOAc (10 mL) maintained at 0° C. was added 4N HCl in EtOAc (5 mL). Reaction was stirred at rt for 16 h.

Progress of reaction was monitored by TLC. After completion, reaction mixture was concentrated, and crude obtained was washed with Et₂O (10 mL) to afford 90 (2.55 g as HCl salt). This was used as such for the next reaction. ¹H NMR (400 MHz, DMSO-d6) δ=8.44 (d, J=4.4 Hz, 1H), 4.27 (q, J=6.8 Hz, 2H), 2.85 (d, J=4.9 Hz, 3H), 1.26 (t, J=7.1 Hz, 3H); LC-MS: m/z 238.9 [M+H]⁺.

### Step-5: Ethyl 2-((4-chloro-N-methylphenyl)sulfonamido)-4-(trifluoromethyl)oxazole-5-carboxylate (92)

To a solution of ethyl 2-(methylamino)-4-(trifluoromethyl)oxazole-5-carboxylate (90) (800 mg, 3.36 mmol, 1 eq) in THF (5 mL) maintained at −78° C. was added LiHMDS (1.3 mL, 8.40 mmol, 2.5 eq). Reaction mixture was stirred at same temperature for 10 min. To this was added 4-chlorobenzenesulfonyl chloride (91) (1.06 g, 5.04 mmol, 1.5 eq) and the reaction was allowed to warm at rt. The reaction mixture was stirred at ambient temperature for 4 h. Progress of reaction was monitored by TLC and LCMS. After completion, reaction mixture was quenched with H₂O (10 mL) and diluted with EtOAc (75 mL). Organic layer was washed with H₂O (2×50 mL) and brine solution (2×50 mL), dried over anhydrous Na₂S₂O₄ and concentrated. The crude obtained was purified by column chromatography (silica 100-200 mesh; 30% EtOAc in Hexanes) to afford 92 (225 mg, 15%). ¹H NMR (400 MHz, DMSO-d₆) δ 8.22 (d, J=7.8 Hz, 1H), 8.15 (d, J=7.8 Hz, 1H), 8.05 (brs, 2H), 4.38-4.26 (m, 2H), 3.64 (brs, 1H), 3.46 (s, 1H), 3.37 (brs, 1H), 1.27 (q, J=7.2 Hz, 3H).

### Step-6: 2-((4-chloro-N-methylphenyl)sulfonamido)-4-(trifluoromethyl)oxazole-5-carboxylic acid (93)

To a solution of ethyl 2-((4-chloro-N-methylphenyl)sulfonamido)-4-(trifluoromethyl)oxazole-5-carboxylate (92) (200 mg, 5.32 mmol, 1 eq) in THF:H₂O (4:1; 5 mL) maintained at 0° C. was added LiGH (32 mg, 1.33 mmol, 2.5 eq). Reaction mixture was stirred at rt for 12 h. Progress of reaction was monitored by TLC. After completion, reaction mixture was concentrated and washed with Et₂O. The crude obtained was acidified with 2N HCl to afford 93 (155 mg with 85% LCMS purity). This was used for the next reaction. LC-MS: m/z 384.6 [M+H]⁺.

### Step-7: Example 53

In line with the General procedure A, acid 93 was coupled with 4-(pyridin-2-yl)thiazol-2-amine (4) to afford Example 53 as an off-white solid. ¹H NMR (400 MHz, DMSO-d₆) δ 13.28 (s, 1H), 8.63 (d, J=4.0 Hz, 1H), 8.09 (d, J=8.0 Hz, 2H), 8.01 (d, J=8.0 Hz, 1H), 7.96 (s, 1H), 7.90 (t, J=8.0 Hz, 1H), 7.79 (d, J=8.4 Hz, 2H), 7.37 (t, J=6.0 Hz, 1H), 3.61 (s, 3H); LC-MS: m/z 544.0 [M+H]⁺; HPLC: 97.4%.

### Synthesis of Example 54

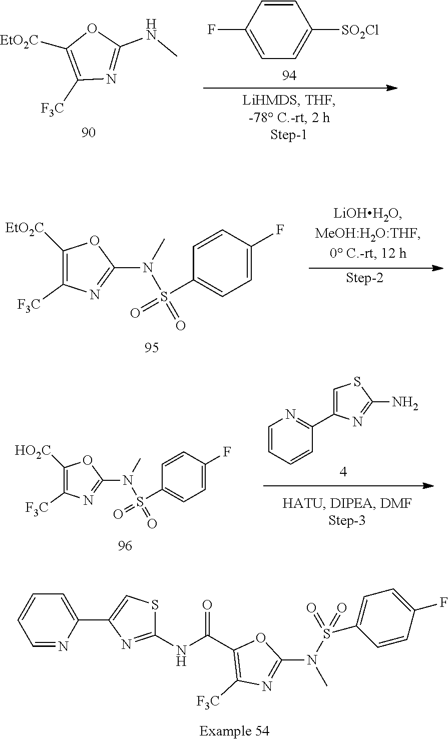

### Step-1: Ethyl 2-((4-fluoro-N-methylphenyl)sulfonamido)-4-(trifluoromethyl)oxazole-5-carboxylate (95)

To a solution of ethyl 2-(methylamino)-4-(trifluoromethyl)oxazole-5-carboxylate (90) (800 mg, 3.36 mmol, 1 eq) in THF (8 mL) maintained at −78° C. was added LiHMDS (1.5 mL, 8.60 mmol, 2.5 eq). Reaction mixture was stirred at same temperature for 10 min. To this was added 4-fluorobenzenesulfonyl chloride (94) (981 mg, 5.04 mmol, 1.5 eq) and the reaction was allowed to warm at rt. The reaction mixture was stirred at ambient temperature for 4 h. Progress of reaction was monitored by TLC and LCMS. After completion, reaction mixture was quenched with H₂O (10 mL) and diluted with EtOAc (75 mL). Organic layer was washed with H₂O (2×50 mL) and brine solution (2×50 mL), dried over anhydrous Na₂S₂O₄ and concentrated.

The crude obtained was purified by column chromatography (silica 100-200 mesh; 30% EtOAc in Hexanes) to afford 95 (155 mg, 12%). ¹H NMR (400 MHz, DMSO-d₆): δ 8.08 (dd, J=4.9, 8.8 Hz, 1H), 7.98 (dd, J=5.1, 9.0 Hz, 1H), 7.53-7.48 (m, 2H), 4.32 (q, 2H), 3.42 (s, 3H), 1.26 (t, 3H); LC-MS: m/z 396.9 [M+H]⁺.

### Step-2: 2-((4-fluoro-N-methylphenyl)sulfonamido)-4-(trifluoromethyl)oxazole-5-carboxylic acid (96)

To a solution of ethyl 2-((4-fluoro-N-methylphenyl)sulfonamido)-4-(trifluoromethyl)oxazole-5-carboxylate (95) (150 mg, 3.78 mmol, 1 eq) in THF:H₂O (4:1; 4 mL) maintained at 0° C. was added LiGH (23 mg, 9.46 mmol, 2.5 eq). Reaction mixture was stirred at rt for 12 h. Progress of reaction was monitored by TLC. After completion, reaction mixture was concentrated and washed with Et₂O. The crude obtained was acidified with 2N HCl to afford 96 (105 mg crude). This was used for the next reaction. ¹H NMR (400 MHz, DMSO-d₆): δ 8.10 (dd, J=5.4, 8.3 Hz, 2H), 7.54 (t, J=8.6 Hz, 2H), 3.44 (s, 3H); LC-MS: m/z 368.8 [M+H]⁺.

### Step-3: Example 54

In line with the General procedure A, acid 96 was coupled with 4-(pyridin-2-yl)thiazol-2-amine (4) to afford Example 53 as an off-white solid. ¹H NMR (400 MHz, DMSO-d₆) δ 13.27 (s, 1H), 8.63 (d, J=4.4 Hz, 1H), 8.18 (m, 2H), 8.02 (d, J=8.0 Hz, 1H), 7.95 (s, 1H), 7.92 (t, J=8.0 Hz, 1H), 7.58 (t, J=8.8 Hz, 2H), 7.37 (t, J=5.6 Hz, 1H), 3.61 (s, 3H); LC-MS: m/z 528.0 [M+H]⁺; HPLC: 98.4%.

### Synthesis of Example 55

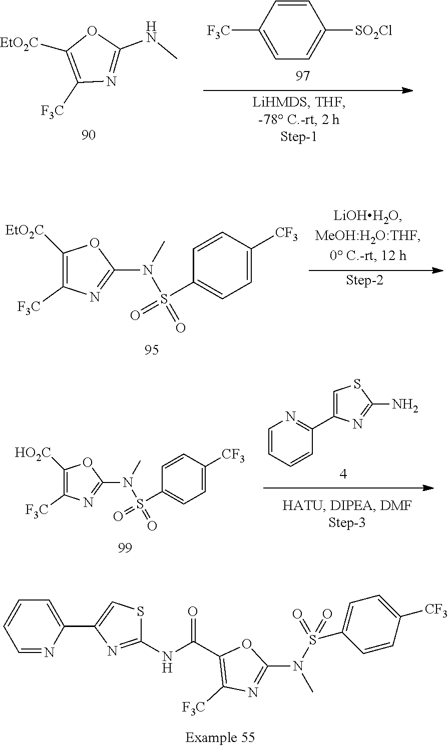

### Step-1: Ethyl 2-((N-methyl-4-(trifluoromethyl)phenyl)sulfonamido)-4-(trifluoromethyl)oxazole-5-carboxylate (98)

To a stirred solution of ethyl 2-(methylamino)-4-(trifluoromethyl)oxazole-5-carboxylate (90) (800 mg, 3.36 mmol, 1 eq) in THF (40 mL), LiHMDS (1M in THF, 8.4 mL, 8.4 mmol, 2.5 eq) was added slowly over a period of 30 min at −78° C. To the resulting mixture, a solution of 4-(trifluoromethyl)benzenesulfonyl chloride (97) (1.22 g, 5.4 mmol, 1.5 eq) was added over a period of 10 min at same temperature and the reaction mixture was stirred at RT for 12 h. The progress of the reaction was monitored by TLC (M.Ph: 20% EtOAc in n-hexane). After completion of reaction, the reaction mixture was quenched with saturated solution of NH₄Cl at −20° C. and extracted with EtOAc (2×250 mL). The combined organic layers were washed with brine, dried over sodium sulfate, filtered and concentrated to dryness. The crude was purified through combi-flash (elution: 10% EtOAc in n-hexane) to afford 98 (320 mg, 19%) as an off-white solid. ¹H NMR (400 MHz, DMSO-d₆) δ 8.23 (d, J=8.0 Hz, 1H), 8.15 (d, J=8.0 Hz, 1H), 8.04 (bs, 2H), 4.34 (q, J=6.8 Hz, 2H), 3.46 (s, 3H), 1.27 (t, J=6.8 Hz, 3H); LC-MS: m/z 445.9 [M+H]⁺.

### Step-2: 2-((N-methyl-4-(trifluoromethyl)phenyl)sulfonamido)-4-(trifluoromethyl)oxazole-5-carboxylic acid (99)

To a stirred solution of ethyl 2-((N-methyl-4-(trifluoromethyl)phenyl)sulfonamido)-4-(trifluoromethyl)oxazole-5-carboxylate (98) (320 mg, 0.71 mmol, 1 eq) in THF:water (1:1, 20 mL), LiOH·H₂O (75 mg, 1.79 mmol, 2.5 eq) was added at RT. The reaction mixture was stirred at RT for 12 h. The progress of the reaction was monitored by TLC (M.Ph: 30% EtOAc in n-hexane). After completion of reaction, the reaction mixture was concentrated to dryness and quenched with ice chilled water. The mixture was extracted with diethyl ether (2×50 mL) and pH of aqueous layer was adjusted to ˜3 by adding 6N HCl solution. The precipitated solid was filtered and dried to afford 99 (150 mg, 51%) as an off-white solid. ¹H NMR (400 MHz, DMSO-d₆) δ 8.25 (d, J=8.4 Hz, 2H), 8.09 (d, J=8.4 Hz, 2H), 3.47 (s, 3H); LC-MS: m/z 418.9 [M+H]⁺.

### Step-3: Example 55

In line with the General procedure A, 2-((N-methyl-4-(trifluoromethyl)phenyl)sulfonamido)-4-(trifluoromethyl)oxazole-5-carboxylic acid (99) (200 mg, 0.47 mmol, 1 eq) was reacted with 4-(pyridin-2-yl)thiazol-2-amine (4) (101 mg, 0.57 mmol, 1.2 eq) to afford Example 55 (120 mg, 44%) as an off-white solid. ¹H NMR (400 MHz, DMSO-d₆) δ 13.29 (s, 1H), 8.61 (d, J=4.4 Hz, 1H), 8.32 (d, J=8.4 Hz, 2H), 8.11 (d, J=8.4 Hz, 2H), 8.02 (d, J=8.0 Hz, 1H), 7.96 (s, 1H), 7.92 (t, J=7.6 Hz, 1H), 7.37 (t, J=6.0 Hz, 1H), 3.65 (s, 3H); LC-MS: m/z 578.05 [M+H]⁺; HPLC: 98.6%.

### Synthesis of Example 56

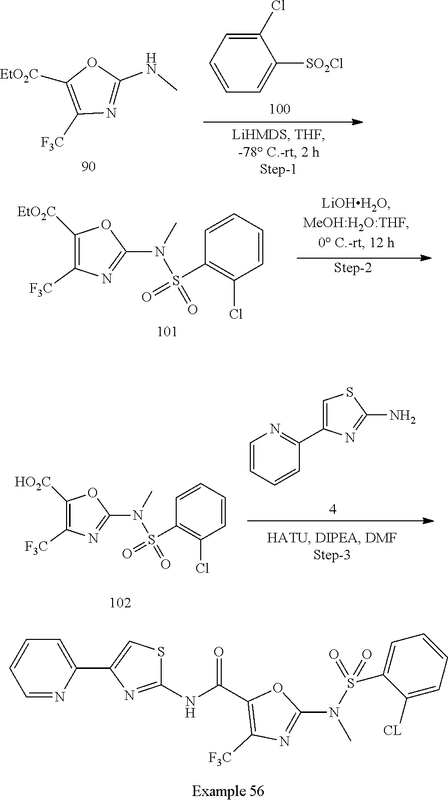

### Step-1: Ethyl 2-((2-chloro-N-methylphenyl)sulfonamido)-4-(trifluoromethyl)oxazole-5-carboxylate (101)

To a stirred mixture of ethyl 2-(methylamino)-4-(trifluoromethyl)oxazole-5-carboxylate (90) (300 mg, 1.26 mmol, 1 eq) in THF (30 mL), LiHMDS (1M in THF, 3.1 mL, 3.15 mmol, 2.5 eq) was added at −78° C. and the mixture was stirred for 30 min at same temperature. To the resulting mixture, 2-chlorobenzenesulfonyl chloride (100) (398 mg, 1.89 mmol, 1.5 eq) was added into the reaction mixture and allowed to warm at RT for 12 h. The progress of the reaction was monitored by TLC (M.Ph: 30% EtOAc in n-hexane). The reaction mixture diluted with water (75 mL) and extracted with EtOAc (2×150 mL). The combined organic layers were washed with brine, dried over sodium sulfate, filtered and concentrated to dryness. The crude was purified through silica gel column chromatography (elution: 15% EtOAc in n-hexane) to afford 101 (0.4 g, 77%) as an off-white solid. ¹H NMR (400 MHz, DMSO-d₆) δ 8.26 (d, J=7.6 Hz, 1H), 7.81-7.75 (m, 2H), 7.67 (t, J=8.0 Hz, 1H), 4.24 (q, J=7.2 Hz, 2H), 3.57 (s, 3H), 1.28 (t, J=7.2 Hz, 3H); LC-MS: m/z 412.9 [M+H]⁺.

### Step-2: 2-((2-Chloro-N-methylphenyl)sulfonamido)-4-(trifluoromethyl)oxazole-5-carboxylic acid (102)

To a stirred solution of ethyl 2-((2-chloro-N-methylphenyl)sulfonamido)-4-(trifluoromethyl)oxazole-5-carboxylate (101) (300 mg, 0.72 mmol, 1 eq) in THF (5 mL), MeOH 20 (5 mL), H₂O (2 mL), LiOH·H₂O (76 mg, 1.82 mmol, 2.5 eq) was added at 0° C. and the reaction mixture was stirred at RT for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 102 (258 mg, 95%) as an off-white solid. ¹H NMR (400 MHz, DMSO-d₆) δ 8.24 (d, J=8.4 Hz, 1H), 7.78-7.75 (m, 2H), 7.66 (t, J=7.6 Hz, 1H), 3.54 (s, 3H); LC-MS: m/z 384.8 [M+H]⁺.

### Step-3: Example 56

In line with the General Procedure A, 2-((2-chloro-N-methylphenyl)sulfonamido)-4-(trifluoromethyl)oxazole-5-carboxylic acid (102) (300 mg, 1.02 mmol, 1 eq) was coupled with 4-(pyridin-2-yl)thiazol-2-amine (4) (216 mg, 1.22 mmol, 1.2 eq) to afford Example 56 (130 mg, 28%) as an off-white solid. ¹H NMR (400 MHz, DMSO-d₆) δ 12.79 (s, 1H), 8.63 (d, J=4.4 Hz, 1H), 8.29 (d, J=8.0 Hz, 1H), 8.02 (d, J=8.0 Hz, 1H), 7.95 (s, 1H), 7.92 (t, J=8.4 Hz, 1H), 7.81-7.75 (m, 2H), 7.68 (t, J=8.4 Hz, 1H), 7.37 (t, J=8.4 Hz, 1H), 3.64 (s, 3H); LC-MS: m/z 544.0 [M+H]⁺; HPLC: 99.7%.

### Synthesis of Example 57

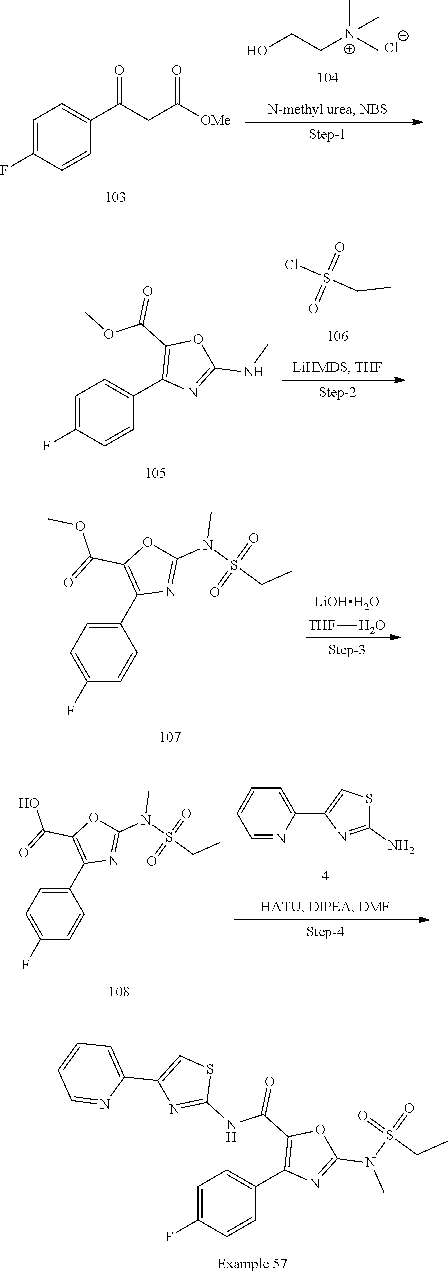

### Step-1: Methyl 4-(4-fluorophenyl)-2-(methylamino)oxazole-5-carboxylate (105)

A mixture of choline chloride (104, 6.80 g, 48.70 mmol) and N methyl urea (3.78 g, 51.02 mmol) was stirred at 60° C. for 30 min in a sealed tube to prepare choline reagent. To the resulting reaction mixture NBS (4.4 g, 24.72 mmol) was added followed by methyl 3-(4-fluorophenyl)-3-oxopropanoate (103, 5.00 g, 25.51 mmol) and N methyl urea (3.21 g, 43.36 mmol) and the reaction mixture was stirred at 65° C. for 18 h. The progress of the reaction was monitored by TLC. After completion of reaction, the reaction mixture was diluted with water. The aqueous layer was extracted with ethyl acetate. The organic layer was dried over anhydrous Na₂SO₄ and concentrated under reduced pressure. The crude compound was purified by 100-200 silica gel column chromatography (30% EtOAc/hexane) to afford compound 105 (1.60 g, 25%) as an off white solid. ¹H NMR (400 MHz, DMSO-d₆) δ 8.02-8.15 (m, 3H), 7.23-7.32 (m, 2H), 3.75 (s, 3H), 2.88 (d, J=4.89 Hz, 3H); LC-MS: m/z 251.5 [M+H]⁺.

### Step-2: Methyl 4-(4-fluorophenyl)-2-(N-methylethylsulfonamido)oxazole-5-carboxylate (107)

To a stirred solution of compound 105 (0.25 g, 1.00 mmol) in THE (10 mL), LiHMDS (1M in THF, 2.0 mL) was added dropwise at −78° C. and the reaction mixture was stirred at that temperature for 30 min. To the resulting reaction mixture ethanesulfonyl chloride (106, 0.19 g, 1.50 mmol) was added slowly and the reaction mixture was stirred at −78° C. for 30 min followed by at RT for 3 h. The progress of the reaction was monitored by TLC. After completion of reaction, the reaction mixture was quenched by saturated NH₄Cl solution at 0° C. The aqueous layer was extracted with ethyl acetate. The organic layer was dried over anhydrous Na₂SO₄ and concentrated under reduced pressure. The crude compound was purified by flash column chromatography (10% EtOAc/hexane) to afford compound 107 (0.27 g, 79%) as an off white solid. ¹H NMR (400 MHz, DMSO-d₆) δ 8.11 (dd, J=6.11, 8.07 Hz, 2H), 7.34 (t, J=8.80 Hz, 2H), 3.84 (s, 3H), 3.67-3.78 (m, 2H), 3.45 (s, 3H), 1.31 (t, J=7.34 Hz, 3H); LC-MS: m/z 343.07 [M+H]⁺.

### Step-3: 4-(4-Fluorophenyl)-2-(N-methylpropan-2-ylsulfonamido)oxazole-5-carboxylic acid (108)

To a stirred solution of compound 107 (0.27 g, 0.79 mmol) in THE (3 mL) and H₂O (2 mL), LiOH·H₂O (0.07 g, 1.58 mmol) was added and the reaction mixture was stirred at 45° C. for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 108 (0.32 g, 84%) as an off white solid. ¹H NMR (400 MHz, DMSO-d₆) δ 13.60 (brs, 1H), 8.13 (dd, J=5.87, 8.80 Hz, 2H), 7.33 (t, J=9.05 Hz, 2H), 3.73 (q, J=7.34 Hz, 2H), 3.44 (s, 3H), 1.31 (t, J=7.34 Hz, 3H); LC-MS: m/z 329.2 [M+H]⁺.

### Step-4: Example 57

In line with the General Procedure A, compound 108 (0.24 g, 0.73 mmol) was coupled with 4-(pyridin-2-yl)thiazol-2-amine (4, 0.13 g, 0.73 mmol) to afford Example 57 (0.195 g, 55%) as an off white solid. ¹H NMR (400 MHz, DMSO-d₆) δ 12.88 (brs, 1H), 8.63 (d, J=3.91 Hz, 1H), 8.24-8.31 (m, 2H), 8.04 (d, J=7.83 Hz, 1H), 7.87-7.94 (m, 2H), 7.33-7.41 (m, 3H), 3.88 (q, J=7.34 Hz, 2H), 3.63 (s, 3H), 1.34 (t, J=7.34 Hz, 3H); LC-MS: m/z 488.15 [M+H]⁺; HPLC: 95.6%.

### Synthesis of Example 58

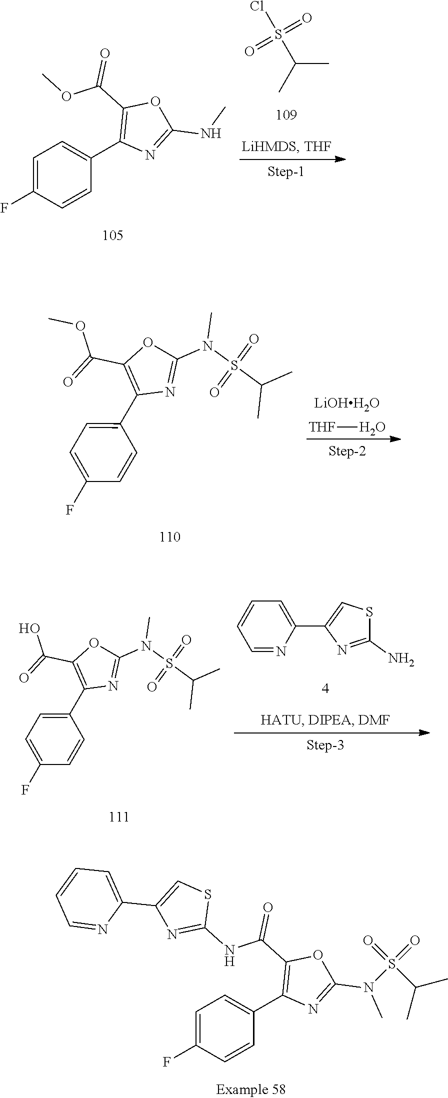

### Step-1: Methyl 4-(4-fluorophenyl)-2-(N-methylpropan-2-ylsulfonamido)oxazole-5-carboxylate (110)

To a stirred solution of compound 105 (0.25 g, 1.00 mmol) in THE (10 mL), LiHMDS (1M in THF, 2.0 mL) was added dropwise at −78° C. and the reaction mixture was stirred at that temperature for 30 min. To the resulting reaction mixture propane-2-sulfonyl chloride (109, 0.21 g, 1.50 mmol) was added slowly and the reaction mixture was stirred at −78° C. for 30 min followed by at RT for 3 h. The progress of the reaction was monitored by TLC. After completion of reaction, the reaction mixture was quenched by saturated NH₄Cl solution at 0° C. The aqueous layer was extracted with ethyl acetate. The organic layer was dried over anhydrous Na₂SO₄ and concentrated under reduced pressure. The crude compound was purified by flash column chromatography (12% EtOAc/hexane) to afford compound 110 (0.39 g, 100%) as an off white solid. ¹H NMR (400 MHz, DMSO-d₆) δ 8.11 (dd, J=5.62, 8.07 Hz, 2H), 7.34 (t, J=8.80 Hz, 2H), 4.04-4.13 (m, 1H), 3.83 (s, 3H), 3.46 (s, 3H), 1.37 (d, J=6.85 Hz, 6H); LC-MS: m/z 357.0 [M+H]⁺.

### Step-2:4-(4-Fluorophenyl)-2-(N-methylpropan-2-ylsulfonamido)oxazole-5-carboxylic acid (111)

To a stirred solution of compound 110 (0.38 g, 1.06 mmol) in THE (3 mL) and H₂O (2 mL), LiOH·H₂O (0.09 g, 2.13 mmol) was added and the reaction mixture was stirred at 45° C. for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 111 (0.32 g, 84%) as an off white solid. ¹H NMR (400 MHz, DMSO-d₆) δ 13.50 (brs, 1H), 8.12 (dd, J=5.87, 8.80 Hz, 2H), 7.32 (t, J=8.80 Hz, 2H), 4.01-4.14 (m, 1H), 3.45 (s, 3H), 1.37 (d, J=6.85 Hz, 6H); LC-MS: m/z 342.9 [M+H]⁺.

### Step-3: Example 58

In line with the General Procedure A, compound 111 (0.30 g, 0.87 mmol) was coupled with 4-(pyridin-2-yl)thiazol-2-amine (7, 0.15 g, 0.87 mmol) to afford Example 58 (0.29 g, 69%) as an off-white solid. ¹H NMR (400 MHz, DMSO-d₆) δ 12.86 (brs, 1H), 8.59-8.66 (m, 1H), 8.27 (dd, J=5.87, 7.34 Hz, 2H), 8.04 (d, J=7.83 Hz, 1H), 7.85-7.95 (m, 2H), 7.31-7.42 (m, 3H), 4.29-4.41 (m, 1H), 3.63 (s, 3H), 1.40 (d, J=6.85 Hz, 6H); LC-MS: m/z 502.45 [M+H]⁺; HPLC: 96.1%.

### Synthesis of Example 59

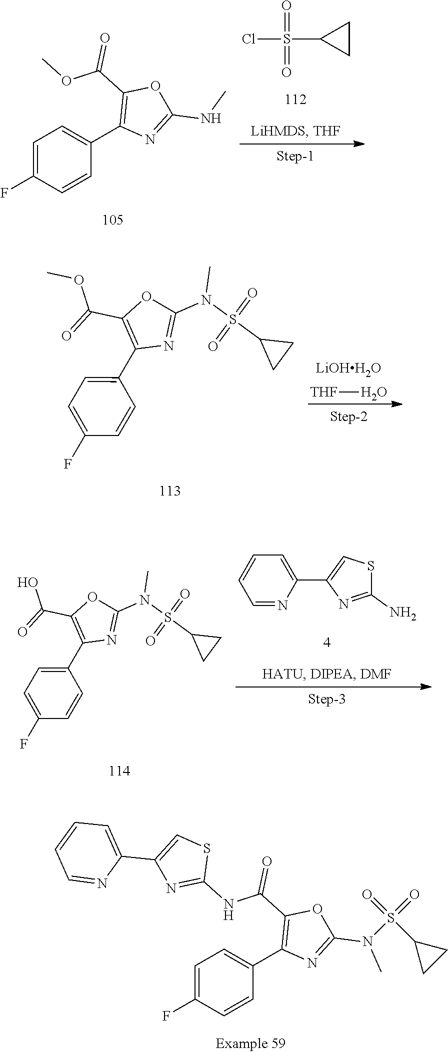

### Step-1: Methyl 4-(4-fluorophenyl)-2-(N-methylcyclopropanesulfonamido)oxazole-5-carboxylate (113)

To a solution of methyl 4-(4-fluorophenyl)-2-(methylamino)oxazole-5-carboxylate (105) (0.500 mg, 2.0 mmol, 1 eq) in THE (4 mL) maintained at −60° C. was added LDA (0.3 mL, 4.0 mmol, 2 eq) and reaction mixture was stirred at the same temperature for 15 min. To this was added cyclopropanesulfonyl chloride (112) and the reaction was stirred at the same temperature for 2 h. Progress of reaction was monitored by TLC. After completion reaction mixture was allowed to warm at room temperature and quenched with H₂O (20 mL). Organic layer was extracted with EtOAc (2×25 mL) washed with brine (20 mL) and concentrated. Crude obtained was purified by column chromatography (silica 100-200 mesh; 20% EtOAc in Hexanes) to afford 113 (155 mg, 22%).

### Step-2: 4-(4-fluorophenyl)-2-(N-methylcyclopropanesulfonamido)oxazole-5-carboxylic acid (114)

To a solution of methyl 4-(4-fluorophenyl)-2-(N-methylcyclopropanesulfonamido) oxazole-5-carboxylate (113) (150 mg, 0.42 mmol, 1 eq) in THF:H₂O:MeOH (2:2:1; 4 mL) was added LiGH (23 mg, 1.06 mmol, 2.5 eq). The reaction mixture was stirred at rt for 12 h. Progress of reaction was monitored by TLC. After completion, the reaction mixture was concentrated, diluted with H₂O and aqueous layer was washed with Et₂O and acidified with 2N HCl solution to afford 114 (135 mg, crude). This was used for the next reaction as such. ¹H NMR (400 MHz, DMSO-d₆) δ=13.62 (brs, 1H), 8.14 (dd, J=5.9, 8.8 Hz, 2H), 7.33 (t, J=9.0 Hz, 3H), 3.46 (s, 3H), 3.27-3.20 (m, 1H), 1.30-1.14 (m, 4H); LCMS: [M+H]⁺=341.0; HPLC: 98%.

### Step-3: Example 59

In line with the General Procedure A, compound 114 was coupled with 4-(pyridin-2-yl)thiazol-2-amine (4) to afford Example 59 (0.29 g, 69%) as an off-white solid. ¹H NMR (400 MHz, DMSO-d₆) δ 12.86 (brs, 1H), 8.59-8.66 (m, 1H), 8.27 (dd, J=5.87, 7.34 Hz, 2H), 8.04 (d, J=7.83 Hz, 1H), 7.85-7.95 (m, 2H), 7.31-7.42 (m, 3H), 4.29-4.41 (m, 1H), 3.63 (s, 3H), 1.40 (d, J=6.85 Hz, 6H); LC-MS: m/z 502.45 [M+H]⁺; HPLC: 96.1%.

### Synthesis of Example 60

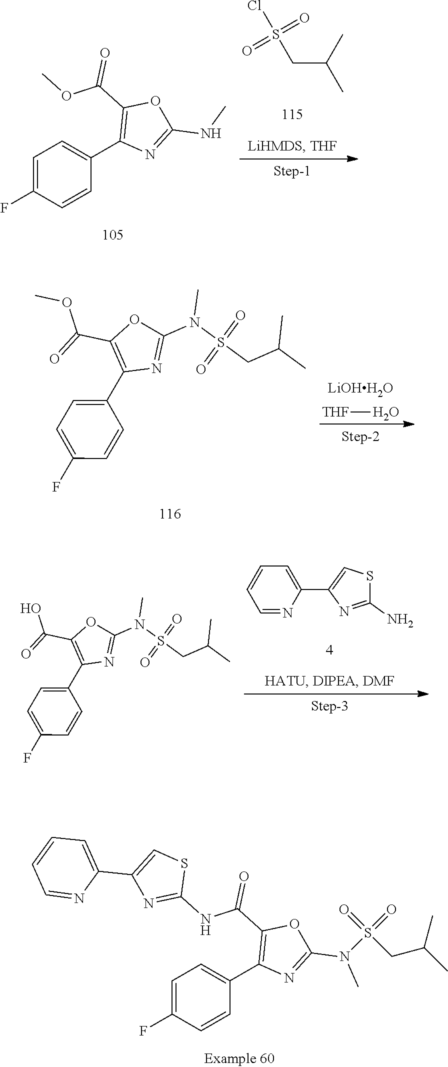

### Step-1: Methyl 2-((N,2-dimethylpropyl)sulfonamido)-4-(4-fluorophenyl)oxazole-5-carboxylate (116)

To a stirred solution of compound 105 (0.25 g, 0.99 mmol, 1 eq) in THF (10 mL), LiHMDS (1M in THF, 2.0 mL, 2 eq) was added dropwise at −78° C. and the reaction mixture was stirred at that temperature for 30 min. To the resulting reaction mixture 2-methylpropane-1-sulfonyl chloride (115, 0.187 g, 1.19 mmol, 1.2 eq) was added slowly and the reaction mixture was stirred at −78° C. for 30 min followed by at RT for 3 h. The progress of the reaction was monitored by TLC. After completion of reaction, the reaction mixture was quenched by saturated NH₄Cl solution at 0° C. The aqueous layer was extracted with ethyl acetate. The organic layer was dried over anhydrous Na₂SO₄ and concentrated under reduced pressure. The crude compound was purified by flash column chromatography (10% EtOAc/hexane) to afford compound 116 (0.27 g, 73%) as an off white solid. LC-MS: m/z 370.95 [M+H]⁺.

### Step-2: 2-((N,2-Dimethylpropyl)sulfonamido)-4-(4-fluorophenyl)oxazole-5-carboxylic acid (117)

To a stirred solution of compound 116 (0.185 g, 0.5 mmol, 1 eq) in THF (3 mL) and H₂O (2 mL), LiOH·H₂O (0.045 g, 1.0 mmol, 2 eq) was added and the reaction mixture was stirred at 45° C. for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 117 (0.130 g, 73%) as an off white solid. LC-MS: m/z 357.1 [M+H]⁺.

### Step-3: Example 60

To a stirred solution of compound 117 (0.125 g, 0.34 mmol, 1 eq) in DMF (5 mL), DIPEA (0.18 mL, 1.02 mmol, 3 eq) and HATU (0.265 g, 0.69 mmol, 2 eq) were added followed by 4-(pyridin-2-yl)thiazol-2-amine (4, 0.074 g, 0.41 mmol, 1.2 eq) and the reaction mixture was stirred at RT for 10 h. The progress of the reaction was monitored by TLC. After completion of reaction, the reaction mixture was diluted with ice cold water. The aqueous layer was extracted with ethyl acetate. The organic layer was washed with water, saturated NaHCO₃ solution and brine, dried over anhydrous Na₂SO₄ and concentrated under reduced pressure. The crude compound was purified by 100-200 silica gel column chromatography (2% MeOH/DCM) to afford Example 60 (0.038 g, 21%) as an off white solid. ¹H NMR (400 MHz, DMSO-d₆) δ 12.88 (brs, 1H), 8.63 (d, J=3.91 Hz, 1H), 8.29-8.25 (m, 2H), 8.04 (d, J=8.0 Hz, 1H), 7.92-7.88 (m, 2H), 7.41-7.34 (m, 3H), 3.76 (d, J=6 Hz, 2H), 3.62 (s, 3H), 3.30-3.23 (m, 1H), 1.10 (t, J=3.6 Hz, 6H); LC-MS: m/z 516.20 [M+H]⁺; HPLC: 99.3%.

### Synthesis of Example 61

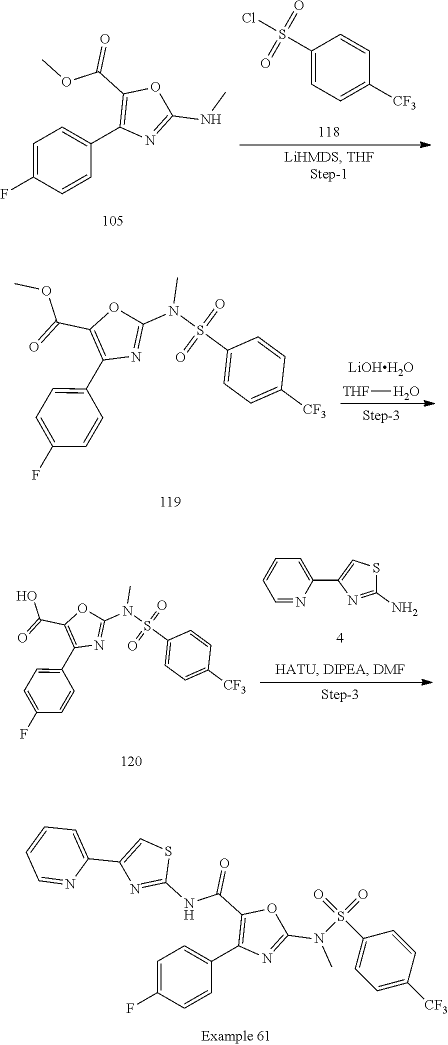

### Step-1: Methyl 4-(4-fluorophenyl)-2-((N-methyl-4-(trifluoromethyl)phenyl)sulfonamido)oxazole-5-carboxylate (119)

To a stirred solution of methyl 4-(4-fluorophenyl)-2-(methylamino)oxazole-5-carboxylate (105) (250 mg, 0.99 mmol, 1 eq) in THE (10 mL), LiHMDS (1M in THF, 1.9 mL, 1.99 mmol, 2 eq) was added at −78° C. After stirring for 5 min at same temperature, 4-(trifluoromethyl)benzenesulfonyl chloride (118) (293 mg, 1.19 mmol, 1.2 eq) was added slowly at −78° C. and the reaction mixture was stirred at RT for 12 h. The progress of the reaction was monitored by TLC (M.Ph: 50% EtOAc in n-hexane). After completion of reaction, the reaction mixture was quenched with sat. NH₄Cl solution. The mixture was extracted with EtOAc (2×100 mL). The combined organic layers were washed with brine, dried over anhydrous Na₂SO₄ and concentrated under reduced pressure and the crude compound was purified by silica gel column chromatography (0-20% EtOAc in n-hexane) to afford 119 (308 mg, 67%) as a white solid. ¹H NMR (400 MHz, DMSO-d₆) δ 8.31 (d, J=8.0 Hz, 2H), 8.11 (d, J=8.4 Hz, 2H), 8.07-8.03 (m, 2H), 7.34 (t, J=8.8 Hz, 2H), 3.85 (s, 3H), 3.54 (s, 3H); LC-MS: m/z 459.10 [M+H]⁺.

### Step-3: 4-(4-Fluorophenyl)-2-((N-methyl-4-(trifluoromethyl)phenyl)sulfonamido)oxazole-5-carboxylic acid (120)

To a stirred mixture of methyl 4-(4-fluorophenyl)-2-((N-methyl-4-(trifluoromethyl)phenyl)sulfonamido)oxazole-5-carboxylate (119) (300 mg, 0.65 mmol, 1 eqv) in THF:water (1.5:1, 10 mL), LiOH·H₂O (55 mg, 1.31 mmol, 2 eq) was added at RT. The reaction mixture was stirred at RT for 2 h. The progress of the reaction was monitored by TLC (M.Ph: 50% EtOAc in n-hexane). After completion of reaction, the solvent was removed under reduced pressure. The residue was diluted with water and adjusted pH to 2-3 with 1N HCl. The solid so obtained was filtered and dried to afford 120 (175 mg, 60%) as an off-white solid. LC-MS: m/z 444.95 [M+H]⁺.

### Step-4: Example 61

In line with the General procedure A, 4-(4-fluorophenyl)-2-((N-methyl-4-(trifluoromethyl)phenyl)sulfonamido)oxazole-5-carboxylic acid (120) (170 mg, 0.67 mmol, 1 eq) was coupled with 4-(pyridin-2-yl)thiazol-2-amine (4) (179 mg, 1.01 mmol, 1.5 eq) to afford Example 61 (66 mg, 29%) as an off-white solid. ¹H NMR (400 MHz, DMSO-d₆) δ 12.89 (s, 1H), 8.61 (bs, 1H), 8.40 (d, J=8.4 Hz, 2H), 8.25 (t, J=8.8 Hz, 2H), 8.13 (d, J=8.8 Hz, 2H), 8.04 (d, J=8.8 Hz, 1H), 7.91-7.88 (m, 2H), 7.40-7.35 (m, 3H), 3.73 (s, 3H); LC-MS: m/z 604.15 [M+H]⁺; HPLC: 98.9%.

### Synthesis of Example 62

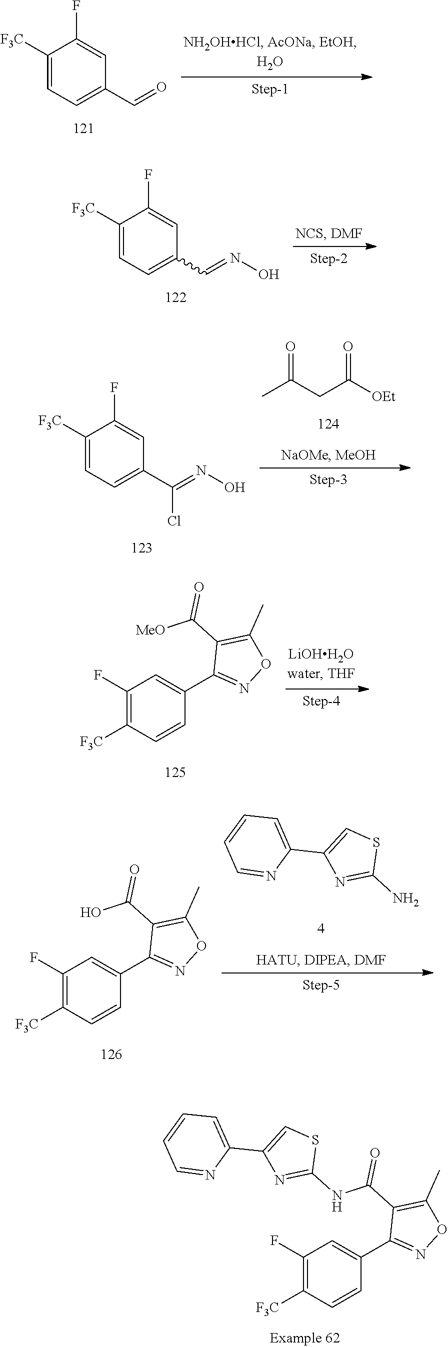

### Step-1: (E/Z)-3-Fluoro-4-(trifluoromethyl)benzaldehyde oxime (122)

To a stirred solution of 3-fluoro-4-(trifluoromethyl)benzaldehyde (121) (1 g, 5.20 mmol, 1 eq) in EtOH:water (1:1, 20 mL), NaOAc (0.51 g, 6.24 mmol, 1.2 eq) was added and stirred for 5 min at RT. To the resulting mixture, NH₂OH·HCl (0.64 g, 6.24 mmol, 1.2 eq) was 20 added and the reaction mixture was stirred for 2 h at 90° C. The progress of the reaction was monitored by TLC (M.Ph: 30% EtOAc in n-hexane). After completion of reaction, the reaction mixture was diluted with water and extracted with EtOAc (2×100 mL). The combined organic layers were washed with brine, dried over anhydrous Na₂SO₄ and concentrated under reduced pressure to afford 122 (1.1 g, 94%) as a white solid. LC-MS: m/z 207.95 [M+H]⁺.

### Step-2: (Z)-3-Fluoro-N-hydroxy-4-(trifluoromethyl)benzimidoyl chloride (123)

To a stirred solution of (E Z)-3-fluoro-4-(trifluoromethyl)benzaldehyde oxime (122) (1 g, 4.8 mmol, 1 eq) in DMF (30 mL), NCS (0.7 g, 5.3 mmol, 1.1 eq) was added at 0° C. and the mixture was stirred at RT for 2 h. The progress of the reaction was monitored by TLC (M.Ph: 30% EtOAc in n-hexane). After completion of reaction, the reaction mixture was diluted with water and extracted with EtOAc (2×150 mL). The combined organic layers was washed with brine, dried over anhydrous Na₂SO₄ and concentrated under reduced pressure to afford 123 (0.3 g, 88%) as a colourless liquid. LC-MS: m/z 242.1 [M+H]⁺.

### Step-3: Methyl 3-(3-fluoro-4-(trifluoromethyl)phenyl)-5-methylisoxazole-4-carboxylate (125)

A mixture of compound 123 (100 mg, 0.76 mmol, 1.0 eq), MeOH (5 mL), NaOMe (30% in MeOH, 103 mg, 1.92 mmol, 2.5 eq) was added at 0° C. followed by ethyl 3-oxobutanoate (124) (200 mg, 0.84 mmol, 1.1 eq). The resulting mixture was stirred at RT for 16 h. After completion of reaction, the reaction mixture was concentrated under reduced pressure to afford 125 (200 mg, 87%) as an off-white solid. ¹H NMR (400 MHz, DMSO-d₆) δ 7.94-7.84 (m, 2H), 7.68 (d, J=7.6 Hz, 1H), 3.73 (s, 3H), 2.73 (s, 3H); LC-MS: m/z 304.10 [M+H]⁻.

### Step-4: 3-(3-Fluoro-4-(trifluoromethyl)phenyl)-5-methylisoxazole-4-carboxylic acid (126)

To a stirred solution of ethyl 3-(3-fluoro-4-(trifluoromethyl)phenyl)-5-methylisoxazole-4-carboxylate (125) (200 mg, 0.66 mmol, 1 eq) in THE (5 mL) and H₂O (5 mL), LiOH·H₂O (22 mg, 0.99 mmol, 1.5 eq) was added at 0° C. and the reaction mixture was stirred at RT for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 126 (180 mg, 95%) as an off-white solid. ¹H NMR (400 MHz, DMSO-d₆) δ 13.29 (s, 1H), 7.93 (t, J=8.0 Hz, 1H), 7.85 (d, J=11.6 Hz, 1H), 7.67 (d, J=8.4 Hz, 2H), 2.67 (s, 3H); LC-MS: m/z 290.0 [M+H]⁻.

### Step-5: Example 62

In line with the General procedure A, 3-(3-fluoro-4-(trifluoromethyl)phenyl)-5-methylisoxazole-4-carboxylic acid (126) (200 mg, 0.69 mmol, 1 eq) was coupled with 4-(pyridin-2-yl)thiazol-2-amine (4) (134 mg, 0.76 mmol, 1.1 eq) to afford Example 62 (80 mg, 26%) as an off-white solid. ¹H NMR (400 MHz, DMSO-d₆) δ 12.67 (s, 1H), 8.61 (bs, 1H), 7.98 (d, J=7.6 Hz, 1H), 7.92-7.88 (m, 2H), 7.87-7.85 (m, 1H), 7.69 (d, J=7.6 Hz, 1H), 7.35 (t, J=8.4 Hz, 1H), 7.40 (s, 1H), 2.69 (s, 3H); LC-MS: m/z 449.15 [M+H]⁺; HPLC: 99.7%.

### Synthesis of Example 63

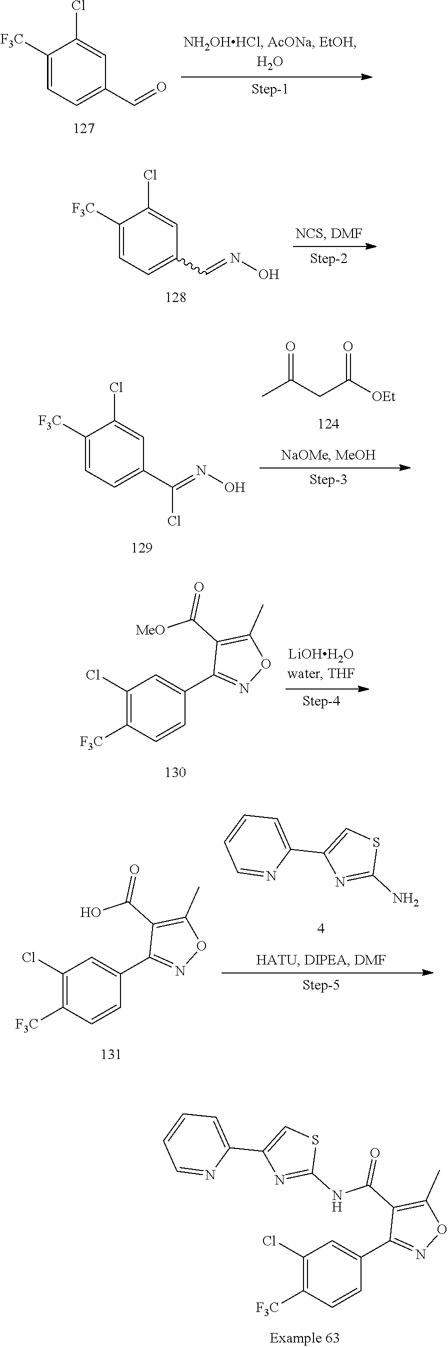

### Step-1: (E/Z)-3-chloro-4-(trifluoromethyl)benzaldehyde oxime (128)

To a stirred solution of 3-chloro-4-(trifluoromethyl)benzaldehyde (127) (500 mg, 2.39 mmol, 1 eq) in EtOH (10 mL), pyridine (0.31 mL, 2.63 mmol, 1.1 eq) was added. After stirring for 5 min at RT, NH₂OH·HCl (180 mg, 2.63 mmol, 1.1 eq) was added and the reaction mixture was stirred for 2 h at RT. The progress of the reaction was monitored by TLC (M.Ph: 30% EtOAc in n-hexane). After completion of reaction, the reaction mixture was diluted with water and extracted with EtOAc (2×100 mL). The combined organic layers were washed with brine, dried over anhydrous Na₂SO₄ and concentrated under reduced pressure to afford 128 (500 mg, 94%) as a white solid. LC-MS: m/z 223.95 [M+H]⁺.

Step-2: (Z)-3-Chloro-N-hydroxy-4-(trifluoromethyl)benzimidoyl chloride (129)

To a stirred solution of (EZ)-3-chloro-4-(trifluoromethyl)benzaldehyde oxime (128) (500 mg, 2.23 mmol, 1 eq) in DMF (5 mL), NCS (330 mg, 2.46 mmol, 1.1 eq) was added at 0° C. and the mixture was stirred at RT for 2 h. The progress of the reaction was monitored by TLC (M.Ph: 30% EtOAc in n-hexane). After completion of reaction, the reaction mixture was diluted with water and extracted with EtOAc (2×100 mL). The combined organic layers was washed with brine, dried over anhydrous Na₂SO₄ and concentrated under reduced pressure to afford 129 (550 mg, 96%) as a colourless liquid. LC-MS: m/z 258.1 [M+H]⁺.

### Step-3: Methyl 3-(3-chloro-4-(trifluoromethyl)phenyl)-5-methylisoxazole-4-carboxylate (130)

A mixture of (Z)-3-chloro-N-hydroxy-4-(trifluoromethyl)benzimidoyl chloride (129) (450 mg, 1.74 mmol, 1.0 eq), MeOH (10 mL), NaOMe (30% in MeOH, 300 mg, 5.23 mmol, 3 eq) was added at 0° C. followed by ethyl 3-oxobutanoate (124) (410 mg, 3.48 mmol, 2 eq). The resulting mixture was stirred at RT for 16 h. After completion of reaction, the reaction mixture was concentrated under reduced pressure to afford 130 (260 mg, 46%) as an off-white solid. LC-MS: m/z 319.8 [M+H]⁻.

### Step-4: 3-(3-Chloro-4-(trifluoromethyl)phenyl)-5-methylisoxazole-4-carboxylic acid (131)

To a stirred solution of methyl 3-(3-chloro-4-(trifluoromethyl)phenyl)-5-methylisoxazole-4-carboxylate (130) (260 mg, 0.81 mmol, 1 eq) in THE (5 mL) and H₂O (2 mL), LiOH·H₂O (68 mg, 1.6 mmol, 2.0 eq) was added at 0° C. and the reaction mixture was stirred at RT for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 131 (200 mg, 80%) as an off-white solid. ¹H NMR (400 MHz, DMSO-d₆) δ 13.33 (s, 1H), 7.95-7.92 (m, 2H), 7.74 (t, J=8.0 Hz, 1H), 2.67 (s, 3H); LC-MS: m/z 303.9 [M−H]⁻.

### Step-5: Example 63

In line with the General procedure A, 3-(3-chloro-4-(trifluoromethyl)phenyl)-5-methylisoxazole-4-carboxylic acid (131) (200 mg, 0.65 mmol, 1 eq) was coupled with 4-(pyridin-2-yl)thiazol-2-amine (4) (140 mg, 0.78 mmol, 1.2 eq) to afford Example 63 (56 mg, 19%) as an off-white solid. ¹H NMR (400 MHz, DMSO-d₆) δ 12.88 (s, 1H), 8.61 (bs, 1H), 8.03-8.01 (m, 2H), 7.93-7.89 (m, 2H) 7.87 (t, J=7.6 Hz, 1H), 7.82 (d, J=8.6 Hz, 1H), 7.35 (t, J=8.0 Hz, 1H), 2.66 (s, 3H); LC-MS: m/z 464.95 [M+H]⁺; HPLC: 99.4%.

### Synthesis of Example 64

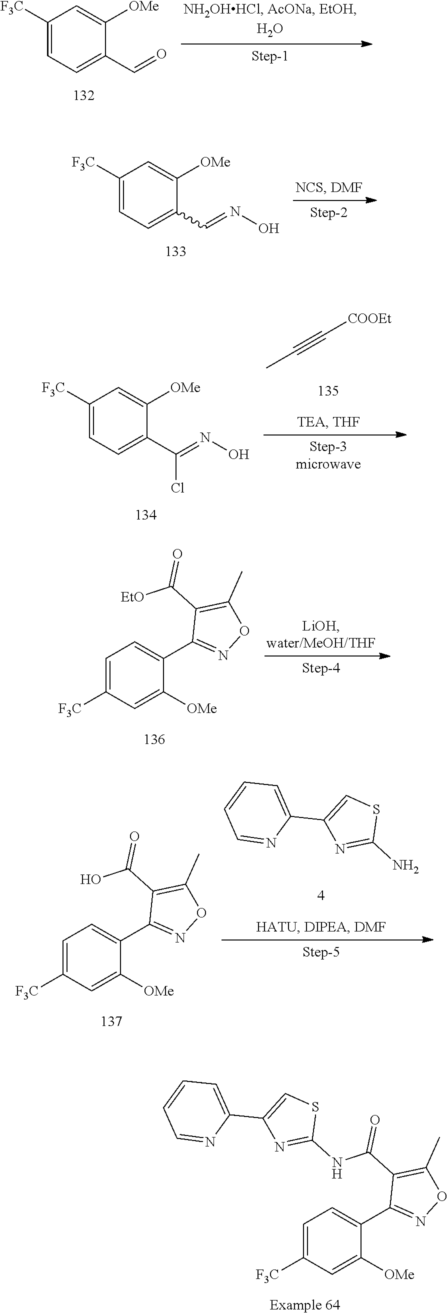

### Step-1: (E/Z)-2-Methoxy-4-(trifluoromethyl)benzaldehyde oxime (133)

To a stirred solution of 2-methoxy-4-(trifluoromethyl)benzaldehyde (132) (500 mg, 2.3 mmol, 1 eq) in EtOH:water (1:1, 20 mL), NaOAc (226 mg, 2.75 mmol, 1.2 eq) was added. After stirring for 5 min at RT, NH₂OH·HCl (193 mg, 2.75 mmol, 1.2 eq) was added and the reaction mixture was stirred for 2 h at 90° C. The progress of the reaction was monitored by TLC (M.Ph: 30% EtOAc in n-hexane). After completion of reaction, the reaction mixture was diluted with water and extracted with EtOAc (2×100 mL). The combined organic layers was washed with brine, dried over anhydrous Na₂SO₄ and concentrated under reduced pressure to afford 133 (500 mg, 99%) as a white solid. LC-MS: m/z 219.95 [M+H]⁺.

### Step-2: (Z)—N-Hydroxy-2-methoxy-4-(trifluoromethyl)benzimidoyl chloride (134)

To a stirred solution of (E Z)-2-methoxy-4-(trifluoromethyl)benzaldehyde oxime (133) (500 mg, 2.28 mmol, 1 eq) in DMF (5 mL), NCS (360 mg, 2.74 mmol, 1.2 eq) was added at 0° C. and the mixture was stirred at RT for 2 h. The progress of the reaction was monitored by TLC (M.Ph: 30% EtOAc in n-hexane). After completion of reaction, the reaction mixture was diluted with water and extracted with EtOAc (2×150 mL). The combined organic layers were washed with brine, dried over anhydrous Na₂SO₄ and concentrated under reduced pressure to afford 134 (500 mg, 87%) as a colourless liquid. LC-MS: m/z 254.0 [M+H]⁺.

### Step-3: Ethyl 3-(2-methoxy-4-(trifluoromethyl)phenyl)-5-methylisoxazole-4-carboxylate (136)

A mixture of (Z)-N-hydroxy-2-methoxy-4-(trifluoromethyl)benzimidoyl chloride (134) (248 mg, 0.98 mmol, 1.2 eq), THE (5 mL), TEA (446 mg, 4.41 mmol, 4.5 eq) and ethyl but-2-ynoate (135) (100 mg, 0.89 mmol, 1.0 eq) was irradiated in a microwave synthesizer at 60° C. for 1 h. The progress of the reaction was monitored by TLC. After completion of reaction, the reaction mixture was concentrated under reduced pressure. The crude compound was purified by combi-flash (30% EtOAc/hexane) to afford 136 (60 mg, 19%) as an off-white solid. LC-MS: m/z 329.95 [M+H]⁻.

### Step-4: 3-(2-Methoxy-4-(trifluoromethyl)phenyl)-5-methylisoxazole-4-carboxylic acid (137)

To a stirred solution of ethyl 3-(2-methoxy-4-(trifluoromethyl)phenyl)-5-methylisoxazole-4-carboxylate (136) (60 mg, 0.18 mmol, 1 eq) in THE (5 mL) and H₂O (2 mL), LiOH·H₂O (19 mg, 0.45 mmol, 2.5 eq) was added at 0° C. and the reaction mixture was stirred at RT for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 137 (30.1 mg, 55%) as an off-white solid. ¹H NMR (400 MHz, DMSO-d₆) δ 12.89 (s, 1H), 7.55 (d, J=8.0 Hz, 1H), 7.41 (d, J=10.8 Hz, 2H), 2.67 (s, 3H); LC-MS: m/z 301.95 [M+H]⁻.

### Step-5: Example 64

In line with the General procedure A, 3-(2-methoxy-4-(trifluoromethyl)phenyl)-5-methylisoxazole-4-carboxylic acid (137) (30 mg, 0.099 mmol, 1 eq) in DCM (5 mL) was coupled with 4-(pyridin-2-yl)thiazol-2-amine (4) (19 mg, 0.10 mmol, 1.1 eq) to afford Example 64 (25 mg, 56%) as an off-white solid. ¹H NMR (400 MHz, DMSO-d₆) δ 12.67 (s, 1H), 8.60 (bs, 1H), 7.93 (d, J=7.6 Hz, 1H), 7.88-7.84 (m, 2H), 7.78 (d, J=7.6 Hz, 1H), 7.49 (d, J=8.4 Hz, 1H), 7.40 (s, 1H), 7.34 (t, J=6.0 Hz, 1H), 3.61 (s, 3H), 2.65 (s, 3H); LC-MS: m/z 461.15 [M+H]⁺; HPLC: 99.7%.

### Synthesis of Example 65

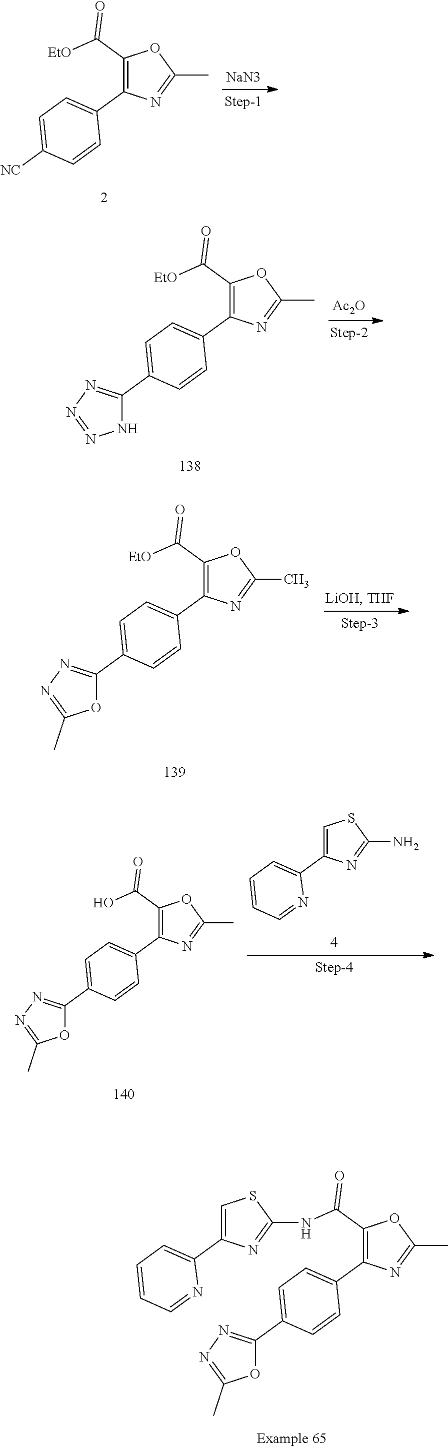

### Step-1: Ethyl 4-(4-(1H-tetrazol-5-yl)phenyl)-2-methyloxazole-5-carboxylate (138)

To a stirred mixture of ethyl 4-(4-cyanophenyl)-2-methyloxazole-5-carboxylate (2; aka Example 30, Step-1) (800 mg, 3.12 mmol, 1 eq) in DMF (12 mL), NaN₃ (609 mg, 9.37 mol, 3 eq) and NH₄Cl (501.4 mg, 9.37 mol, 3 eq) were added at RT and the mixture was stirred at 120° C. for 16 h. The progress of the reaction was monitored by TLC (M.Ph: 5% MeOH in DCM). After completion of reaction, the reaction mixture was diluted with water. The aqueous layer was extracted with EtOAc (2×150 mL). The organic layer was washed with brine, dried over anhydrous Na₂SO₄ and concentrated under reduced pressure. The crude compound was purified by flash column chromatography (0-50% EtOAc in n-hexane) to afford 138 (700 mg, 75%) as a brown solid. LC-MS: m/z 300.60 [M+H]⁺.

### Step-2: Ethyl 2-methyl-4-(4-(5-methyl-1,3,4-oxadiazol-2-yl)phenyl)oxazole-5-carboxylate (139)

A mixture of ethyl 4-(4-(1H-tetrazol-5-yl)phenyl)-2-methyloxazole-5-carboxylate (138) (200 mg, 0.668 mmol, 1 eq) and Ac₂O (50 mL) was stirred at 140° C. for 5 h. The progress of the reaction was monitored by TLC (M.Ph: 5% MeOH in DCM). After completion of reaction, the reaction mixture concentrated under reduced pressure and the crude compound was purified by flash column chromatography (0-80% EtOAc in n-hexane) to afford 139 (186 mg, 89%) as a brown solid. LC-MS: m/z 314.3 [M+H]⁺.

### Step-3: 2-Methyl-4-(4-(5-methyl-1,3,4-oxadiazol-2-yl)phenyl)oxazole-5-carboxylic acid (140)

To a stirred solution of ethyl 2-methyl-4-(4-(5-methyl-1,3,4-oxadiazol-2-yl)phenyl)oxazole-5-carboxylate (139) (180 mg, 0.57 mmol, 1 eq) in THF:water (1:1, 10 mL), LiOH·H₂O (48.2 mg, 1.15 mmol, 2 eq) was added at RT. The reaction mixture was stirred at RT for 2 h. The progress of the reaction was monitored by TLC (M.Ph: 10% MeOH in DCM). After completion of reaction, the reaction mixture was concentrated to dryness and quenched with ice chilled water and pH of aqueous layer was adjusted to ˜3 by adding 6N HCl solution. The precipitated solid was filtered and dried to afford 140 (160 mg, 98%) as an off-white solid. ¹H NMR (400 MHz, DMSO-d₆) δ 13.1 (s, 1H), 8.26 (d, J=8.4 Hz, 2H), 8.06 (d, J=9.2 Hz, 2H), 2.60 (s, 3H), 2.55 (s, 3H); LC-MS: m/z 285.9 [M+H]⁺.

### Step-4: Example 65

In line with the General Procedure A, 2-methyl-4-(4-(5-methyl-1,3,4-oxadiazol-2-yl)phenyl)oxazole-5-carboxylic acid (140) (160 mg, 0.56 mmol, 1 eq) was coupled with 4-(pyridin-2-yl)thiazol-2-amine (4) (99.3 mg, 0.56 mmol, 1.0 eq) to afford Example 65 (29 mg, 11%) as an off-white solid. ¹H NMR (400 MHz, DMSO-d₆) δ 12.83 (s, 1H), 8.63 (bs, 1H), 8.40 (d, J=6.8 Hz, 2H), 8.01 (bs, 1H), 8.11 (d, J=7.2 Hz, 2H), 8.01 (bs, 1H), 7.94-7.91 (m, 2H), 2.60 (s, 3H), 2.55 (s, 3H); LC-MS: m/z 445.2 [M+H]⁺; HPLC: 99.8%.

### Synthesis of Example 66

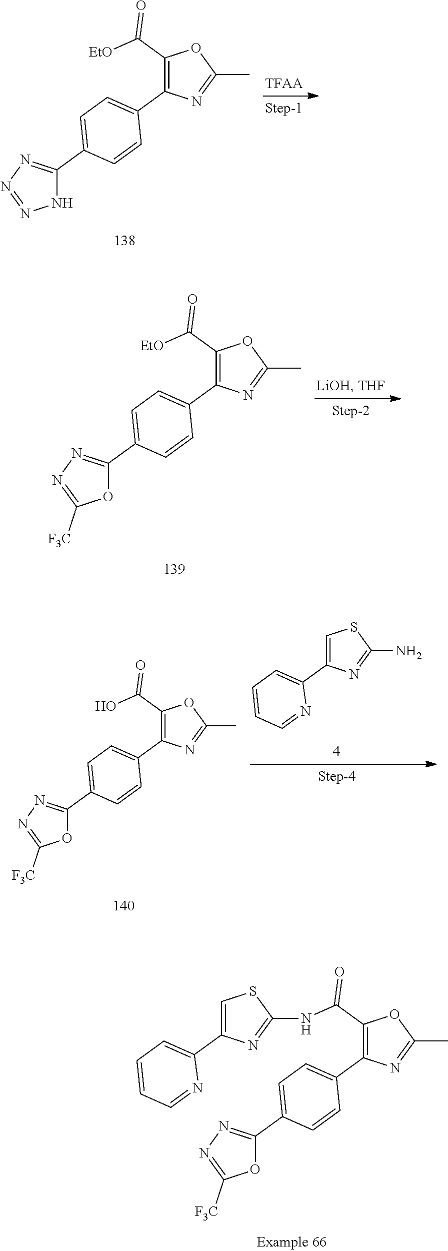

### Step-1: 4-(4-(1H-Tetrazol-5-yl)phenyl)-2-methyloxazole-5-carboxylic acid (139)

To a stirred solution of methyl 4-(4-(1H-tetrazol-5-yl)phenyl)-2-methyloxazole-5-carboxylate (138) (210 mg, 0.73 mmol, 1 eq) in THF:MeOH:H₂O (1:1:1, 9 mL), LiOH·H₂O (61.8 mg, 1.47 mmol, 2 eq) was added at 0° C. and the reaction mixture was stirred at 50° C. for 2 h. The progress of the reaction was monitored by TLC (M.Ph: 15% MeOH in DCM). After completion of reaction, the reaction mixture was concentrated under reduced pressure. The solid residue was diluted with water and pH was adjusted to ˜3 by adding 1N HCl solution and extracted with 20% MeOH in DCM. The combined organic layers was dried over anhydrous Na₂SO₄ and concentrated under reduced pressure to afford compound 139 (144 mg, 72%) as an off-white solid. ¹H NMR (400 MHz, DMSO-d₆) δ 8.26 (d, J=8.4, 2H), 8.18 (d, J=8.4, 2H, 2.55 (s, 3H); LC-MS: m/z 271.9 [M+H]⁺.

### Step-2: 2-Methyl-4-(4-(5-(trifluoromethyl)-1,3,4-oxadiazol-2-yl)phenyl)oxazole-5-carboxylic acid (140)

A mixture of 4-(4-(1H-tetrazol-5-yl)phenyl)-2-methyloxazole-5-carboxylic acid (139) (50 mg, 0.18 mmol, 1 eq) and TFAA (2 mL) was stirred at 80° C. for 2 h. The progress of the reaction was monitored by TLC (M.Ph: 5% MeOH in DCM). After completion of reaction, the reaction mixture was diluted with water and extracted with EtOAc (2×50 mL). The combined organic layers was washed with brine, dried over anhydrous Na₂SO₄ and concentrated under reduced pressure to afford 140 (35 mg, 56%) as a white solid. LC-MS: m/z 339.95 [M+H]⁻.

### Step-3: Example 66

In line with the General Procedure A, 2-methyl-4-(4-(5-(trifluoromethyl)-1,3,4-oxadiazol-2-yl)phenyl)oxazole-5-carboxylic acid (140) (110 mg, 0.32 mmol, 1 eq) was coupled with 4-(pyridin-2-yl)thiazol-2-amine (4) (63 mg, 0.35 mmol, 1.1 eq) to afford Example 66 (30 mg, 19%) as an off-white solid. ¹H NMR (400 MHz, DMSO-d₆) δ 12.97 (s, 1H), 8.63 (bs, 1H), 8.45 (d, J=8.0 Hz, 2H), 8.24 (d, J=8.4 Hz, 2H), 8.06 (d, J=7.6 Hz, 1H), 7.98-7.96 (m, 2H), 7.40 (bs, 1H), 2.63 (s, 3H); LC-MS: m/z 498.9 [M+H]⁺; HPLC: 98.3%.

### Synthesis of Example 67

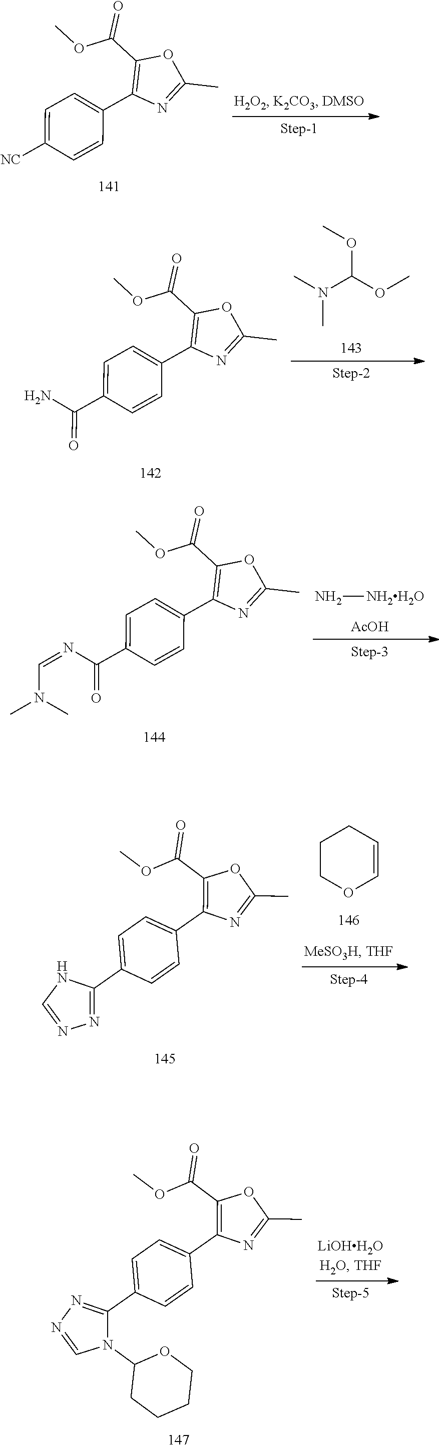

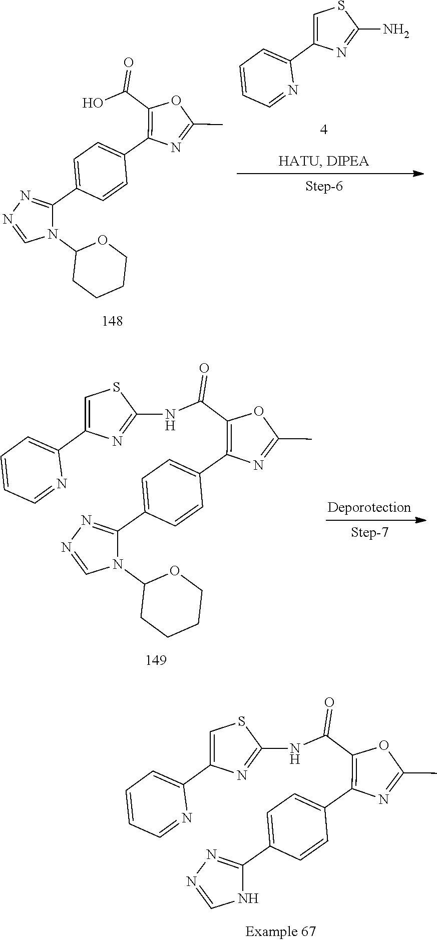

### Step-1: Methyl 4-(4-carbamoylphenyl)-2-methyloxazole-5-carboxylate (142)

A mixture of methyl 4-(4-cyanophenyl)-2-methyloxazole-5-carboxylate (141; synthesized using methodology to that for Step-1, Example 30) (0.5 g, 2.06 mmol, 1 eq) in DMSO (15 mL), K₂CO₃ (0.71 g, 5.16 mmol, 2.5 eq) and H₂O₂(30%, 1.17 mL, 10.3 mmol, 5 eq) was added at RT and the mixture was stirred at 90° C. for 3 h. The progress of the reaction was monitored by TLC (M.Ph: 50% EtOAc in n-hexane). After completion of reaction, the reaction mixture was diluted with ice cold water. The aqueous layer was extracted with EtOAc (2×75 mL). The organic layer was washed thoroughly with cold water and brine, dried over anhydrous Na₂SO₄ and concentrated under reduced pressure. The crude compound was purified by silica gel column chromatography (30% EtOAc/hexane) to afford 142 (0.37 g, 69%) as an off-white solid. LC-MS: m/z 260.9 [M+H]⁺.

### Step-2: Methyl (Z)-4-(4-(((dimethylamino)methylene)carbamoyl)phenyl)-2-methyloxazole-5-carboxylate (144)

A mixture of methyl 4-(4-carbamoylphenyl)-2-methyloxazole-5-carboxylate (142) (0.3 g, 1.15 mmol, 1 eq) and N,N-DMFDMA (143, 10 mL) was stirred at 95° C. for 2 h. The progress of the reaction was monitored by TLC (M.Ph: 50% EtOAc in n-hexane). After completion of reaction, the reaction mixture was concentrated under reduced pressure to afford crude compound (144) (0.32 g) as a brown solid, which was used in the next step without further purification. LC-MS: m/z 316.2 [M+H]⁺.

### Step-3: Methyl 4-(4-(4H-1,2,4-triazol-3-yl)phenyl)-2-methyloxazole-5-carboxylate (145)

To a stirred solution of methyl (Z)-4-(4-(((dimethylamino)methylene)carbamoyl)phenyl)-2-methyloxazole-5-carboxylate (144) (0.3 g, 0.95 mmol, 1 eq) in AcOH (10 mL), N₂H₄.H₂O (0.32 g, 4.7 mmol, 5 eq) was added at RT and the mixture was stirred at 95° C. for 2 h. The progress of the reaction was monitored by TLC (M.Ph: 50% EtOAc in n-hexane). After completion of reaction, the reaction mixture was concentrated under reduced pressure to afford 145 (0.4 g, 68%) as yellow solid. LC-MS: m/z 284.95 [M+H]⁺.

### Step-4: Methyl 2-methyl-4-(4-(4-(tetrahydro-2H-pyran-2-yl)-4H-1,2,4-triazol-3-yl)phenyl)oxazole-5-carboxylate (147)

To a stirred solution of methyl 4-(4-(4H-1,2,4-triazol-3-yl)phenyl)-2-methyloxazole-5-carboxylate (145) (0.4 g, 1.4 mmol, 1 eq) in THF (10 mL), CH₃SO₃H (67 mg, 0.69 mmol, 0.5 eq) and 3,4-dihydro-2H-pyran (146) (0.59 g, 7.04 mmol, 5 eq) were added at RT and the reaction mixture was stirred at 60° C. for 20 h. The progress of the reaction was monitored by TLC (M.Ph: 50% EtOAc in n-hexane). After completion of reaction, the reaction mixture was diluted with ice cold water. The aqueous layer was extracted with EtOAc (2×75 mL). The organic layer was washed thoroughly with cold water and brine, dried over anhydrous Na₂SO₄ and concentrated under reduced pressure. The crude compound was purified by silica gel column chromatography (30% EtOAc/hexane) to afford 147 (0.3 g, 59%) as an off-white solid. LC-MS: m/z 369.0 [M+H]⁻.

### Step-5: 2-Methyl-4-(4-(4-(tetrahydro-2H-pyran-2-yl)-4H-1,2,4-triazol-3-yl)phenyl)oxazole-5-carboxylic acid (148)

To a stirred solution of methyl 2-methyl-4-(4-(4-(tetrahydro-2H-pyran-2-yl)-4H-1,2,4-triazol-3-yl)phenyl)oxazole-5-carboxylate (147) (300 mg, 0.81 mmol, 1 eq) in THF:water (1:1, 10 mL), LiOH·H₂O (28 mg, 1.22 mmol, 1.5 eq) was added at RT. The reaction mixture was stirred at RT for 12 h. The progress of the reaction was monitored by TLC (M.Ph: 30% EtOAc in n-hexane). After completion of reaction, the reaction mixture was concentrated to dryness and quenched with ice chilled water. The mixture was extracted with diethyl ether (2×50 mL) and pH of aqueous layer was adjusted to ˜3 by adding 6N HCl solution. The precipitated solid was filtered and dried to afford 148 (250 mg, 92%) as an off-white solid. LC-MS: m/z 355.1 [M+H]⁺.

### Step-6: 2-methyl-N-(4-(pyridin-2-yl)thiazol-2-yl)-4-(4-(4-(tetrahydro-2H-pyran-2-yl)-4H-1,2,4-triazol-3-yl)phenyl)oxazole-5-carboxamide (149)

In line with the General Procedure A, 2-methyl-4-(4-(4-(tetrahydro-2H-pyran-2-yl)-4H-1,2,4-triazol-3-yl)phenyl)oxazole-5-carboxylic acid (148) (0.25 g, 0.73 mmol, 1 eq) was coupled with 4-(pyridin-2-yl)thiazol-2-amine (4, 0.157 g, 0.88 mmol, 1.2 eq) to afford 149 (0.250 g, 92%) as an off-white solid. LC-MS: m/z 514.1 [M+H]⁺.

### Step-7: Example 67

A mixture of 2-methyl-N-(4-(pyridin-2-yl)thiazol-2-yl)-4-(4-(4-(tetrahydro-2H-pyran-2-yl)-4H-1,2,4-triazol-3-yl)phenyl)oxazole-5-carboxamide (149) (0.2 g, 0.38 mmol, 1 eq) and 4M dioxane HCl (10 mL) was stirred at RT for 12 h. The progress of the reaction was monitored by TLC (M.Ph: 5% DCM in MeOH). After completion of reaction, the reaction mixture was concentrated to dryness under reduced pressure and the residue was basified with saturated NaHCO₃ solution. The aqueous layer was extracted with ethyl acetate (2×50 mL). The organic layer was washed thoroughly with cold water and brine, dried over anhydrous Na₂SO₄ and concentrated under reduced pressure. The crude compound was purified by flash column chromatography (5% MeOH/DCM) to afford Example 167 (10 mg, 6%) as an off-white solid. ¹H NMR (400 MHz, DMSO-d₆) δ 14.15 (s, 1H), 12.87 (s, 1H), 8.63 (bs, 1H), 8.31 (d, J=6.8 Hz, 2H), 8.14 (d, J=8.0 Hz, 2H), 8.03 (d, J=7.6 Hz, 1H), 7.92-7.89 (m, 2H), 7.31 (t, J=7.6 Hz, 1H), 2.61 (s, 3H); LC-MS: m/z 430.0 [M+H]⁺; HPLC: 98.0%.

### Synthesis of Example 68

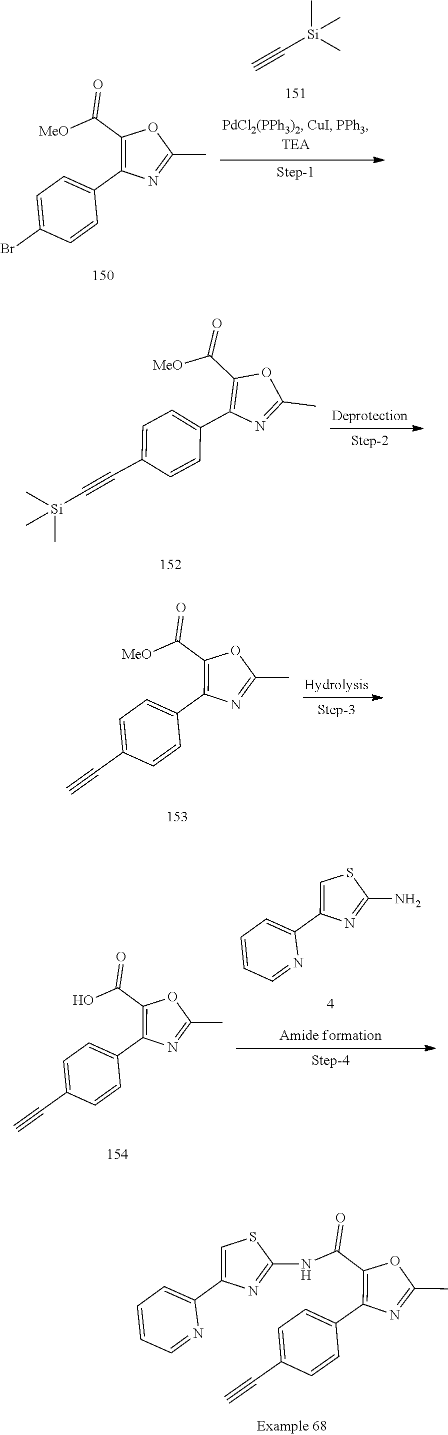

### Step-1: Methyl 2-methyl-4-(4-((trimethylsilyl)ethynyl)phenyl)oxazole-5-carboxylate (152)

To a stirred solution of methyl 4-(4-bromophenyl)-2-methyloxazole-5-carboxylate (150) (3 g, 10.6 mmol, 1 eq) in TEA (90 mL, 30 vol.), CuI (201 mg, 1.06 mmol, 0.1 eq) and PPh₃ (194 mg, 0.74 mmol, 0.07 eq) were added and degassed the mixture for 10 min with the help of nitrogen. Then PdCl₂(PPh₃)₂(2.2 g, 3.13 mmol, 0.3 eq) and trimethylsilyl acetylene (151) (1.4 g, 15.9 mmol, 1.5 eq) were added into the solution at RT. After the reaction mixture being degassed for 10 min with the help of nitrogen, it was stirred for 2 h at 90° C. The progress of the reaction was monitored by TLC (M.Ph: 30% EtOAc in n-hexane). After completion of reaction, the reaction mixture was diluted with water and extracted with EtOAc (2×200 mL). The combined organic layers was washed with brine, dried over anhydrous Na₂SO₄ and concentrated under reduced pressure and the crude compound was purified by silica gel column chromatography (0-10% EtOAc in n-hexane) to afford 152 (2.8 g, 88%) as a white solid. LC-MS: m/z 313.95 [M+H]⁺.

### Step-2: Methyl 4-(4-ethynylphenyl)-2-methyloxazole-5-carboxylate (153)

To a stirred solution of methyl 2-methyl-4-(4-((trimethylsilyl)ethynyl)phenyl)oxazole-5-carboxylate (152) (2.8 g, 8.93 mmol, 1 eq) in MeOH (45 mL), K₂CO₃ (2.46 g, 17.86 mmol, 2 eq) was added at 0° C. and the mixture was stirred at RT for 2 h. The progress of the reaction was monitored by TLC (M.Ph: 30% EtOAc in n-hexane). After completion of reaction, the reaction mixture was diluted with water and extracted with EtOAc (2×150 mL). The combined organic layers was washed with brine, dried over anhydrous Na₂SO₄ and concentrated under reduced pressure and the crude compound was purified by silica gel column chromatography (0-10% EtOAc in n-hexane) to afford 153 (1.5 g, 70%) as a colourless liquid. LC-MS: m/z 242.0 [M+H]⁺.

### Step-3: 4-(4-Ethynylphenyl)-2-methyloxazole-5-carboxylic acid (154)

To a stirred solution of methyl 4-(4-ethynylphenyl)-2-methyloxazole-5-carboxylate (153) (0.4 g, 1.66 mmol, 1 eq) in THE (5 mL) and H₂O (2 mL), LiOH·H₂O (0.112 g, 2.48 mmol, 1.5 eq) was added at 0° C. and the reaction mixture was stirred at RT for 2 h. The progress of the reaction was monitored by TLC. After completion of reaction, the reaction mixture was concentrated under reduced pressure. The solid residue was diluted with water and pH was adjusted to ˜3 by adding 1N HCl solution. The precipitated solid was filtered, washed with water and dried under reduced pressure to afford compound 154 (0.34 g, 90%) as an off-white solid. LC-MS: m/z 228.1 [M+H]⁻.

### Step-4: Example 68

In line with the General Procedure A, 4-(4-ethynylphenyl)-2-methyloxazole-5-carboxylic acid (154) (170 mg, 0.74 mmol, 1 eq) was coupled with 4-(pyridin-2-yl)thiazol-2-amine (4) (145 mg, 0.82 mmol, 1.1 eq) to afford Example 68 (175 mg, 61%) as an off-white solid. ¹H NMR (400 MHz, DMSO-d₆) δ 12.86 (s, 1H), 8.63 (bs, 1H), 8.22 (d, J=8.4 Hz, 2H), 8.03 (d, J=7.6 Hz, 1H), 7.92-7.89 (m, 2H), 7.62 (d, J=8.4 Hz, 2H), 7.37 (t, J=5.6 Hz, 1H), 4.34 (s, 1H), 2.60 (s, 3H): LC-MS: m/z 386.95 [M+H]⁺; HPLC: 97.5%.

### Synthesis of Example 69

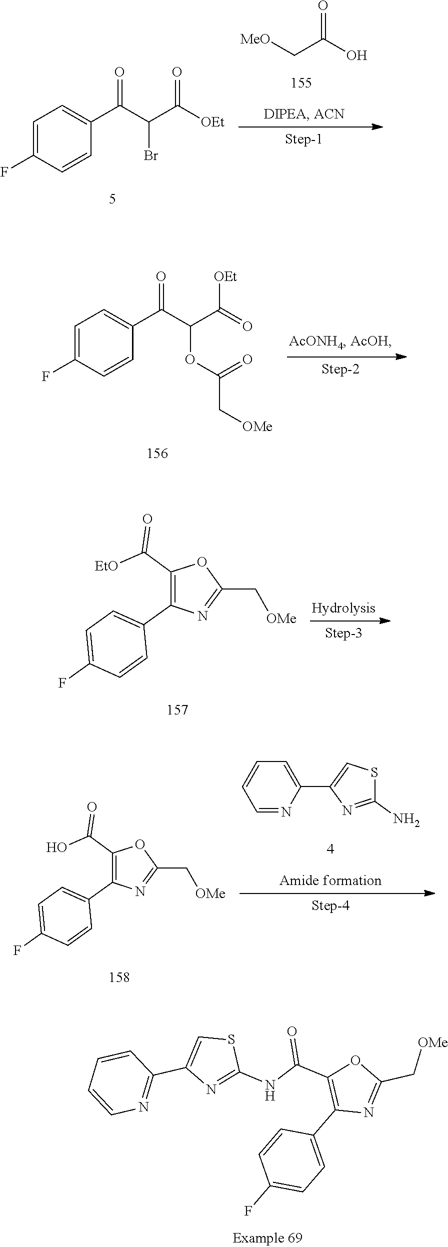

### Step-1: Ethyl 3-(4-fluorophenyl)-2-(2-methoxyacetoxy)-3-oxopropanoate (156)

To a stirred solution of ethyl 2-bromo-3-(4-fluorophenyl)-3-oxopropanoate (5) (500 mg, 1.73 mmol, 1 eq) in ACN (20 mL), DIPEA (0.589 mg, 3.45 mmol, 2 eq) and methoxy acetic acid (155) (311 mg, 3.45 mmol, 2 eq) were added at RT. The reaction mixture was refluxed for 12 h. The progress of the reaction was monitored by TLC (M.Ph: 20% EtOAc in n-hexane).

After completion of reaction, the reaction mixture was concentrated under reduced pressure. The crude compound was purified by combi-flash (0-15% EtOAc in n-hexane) to afford 156 (320 mg, 62%) as a viscous mass. ¹H NMR (400 MHz, CDCl₃) δ 8.02-8.09 (m, 2H), 7.15-7.22 (m, 2H), 6.36 (s, 1H), 4.28-4.18 (m, 4H), 3.47 (s, 3H), 1.23 (t, J=7.6 Hz, 3H).

### Step-2: Ethyl 4-(4-fluorophenyl)-2-(methoxymethyl)oxazole-5-carboxylate (157)

To a stirred solution of ethyl 3-(4-fluorophenyl)-2-(2-methoxyacetoxy)-3-oxopropanoate (156) (310 mg, 1.04 mmol, 1 eq) in AcOH (10 mL), ammonium acetate (160 mg, 2.08 mmol, 2 eq) was added at 0° C. The reaction mixture was stirred at 110° C. for 6 h. The progress of the reaction was monitored by TLC (M.Ph: 30% EtOAc in n-hexane). After completion of reaction, the reaction mixture was concentrated to dryness under reduced pressure.

The resulting residue was diluted with water and extracted with diethyl ether (2×75 mL). The combined organic layers was washed with brine, dried over anhydrous Na₂SO₄ and concentrated under reduced pressure. The crude compound was purified by combi-flash (0-10% EtOAc in n-hexane) to afford 157 (162 mg, 56%) as a viscous mass. ¹H NMR (400 MHz, CDCl₃) δ 8.15-8.11 (m, 2H), 7.17 (t, J=8.6 Hz, 2H), 4.64 (s, 2H), 4.45 (q, J=7.6 Hz, 2H), 3.55 (s, 3H), 1.43 (t, J=6.8 Hz, 3H); LC-MS: m/z 280.00 [M+H]⁺.

### Step-3: 4-(4-Fluorophenyl)-2-(methoxymethyl)oxazole-5-carboxylic acid (158)

To a stirred mixture of methyl 4-(4-fluorophenyl)-2-(methoxymethyl)oxazole-5-carboxylate (157) (160 mg, 0.57 mmol, 1 eq) in THF:H₂O (2:1, 15 mL), LiOH·H₂O (60 mg, 1.42 mmol, 2.0 eq) was added at RT. The reaction mixture was stirred at RT for 12 h. The progress of the reaction was monitored by TLC (M.Ph: 20% EtOAc in n-hexane). The reaction mixture was concentrated under reduced pressure. The resulting residue was diluted with water (50 mL). The aqueous layer was acidified with 6N HCl and extracted with EtOAc (2×75 mL). The combined organic layers were washed with brine, dried over sodium sulfate, filtered and concentrated to dryness to afford titled compound 158 (143 mg, 71%) as an off-white solid. ¹H NMR (400 MHz, DMSO-d₆) δ 8.02-7.99 (m, 2H), 7.28 (t, J=8.8 Hz, 2H), 4.56 (s, 2H), 3.34 (s, 3H); LC-MS: m/z 251.95 [M+H]⁺.

### Step-4: Example 69

In line with General Procedure A, 4-(4-fluorophenyl)-2-(methoxymethyl)oxazole-5-carboxylic acid (158) (100 mg, 0.39 mmol, 1 eq) was coupled with 4-(pyridin-2-yl)thiazol-2-amine (4) (77.5 mg, 0.43 mmol, 1.1 eq) to afford Example 69 (35 mg, 21%) as an off-white solid. ¹H NMR (400 MHz, DMSO-d₆) δ 12.96 (bs, 1H), 8.65-8.60 (m, 1H), 8.29-8.22 (m, 2H), 8.02 (d, J=8.0 Hz, 1H), 7.76-7.88 (m, 2H), 7.42-7.32 (m, 3H), 4.67 (s, 2H), 3.47 (s, 3H); LC-MS: m/z 411.50 [M+H]⁺, HPLC: 99.8%.

### Synthesis of Example 70

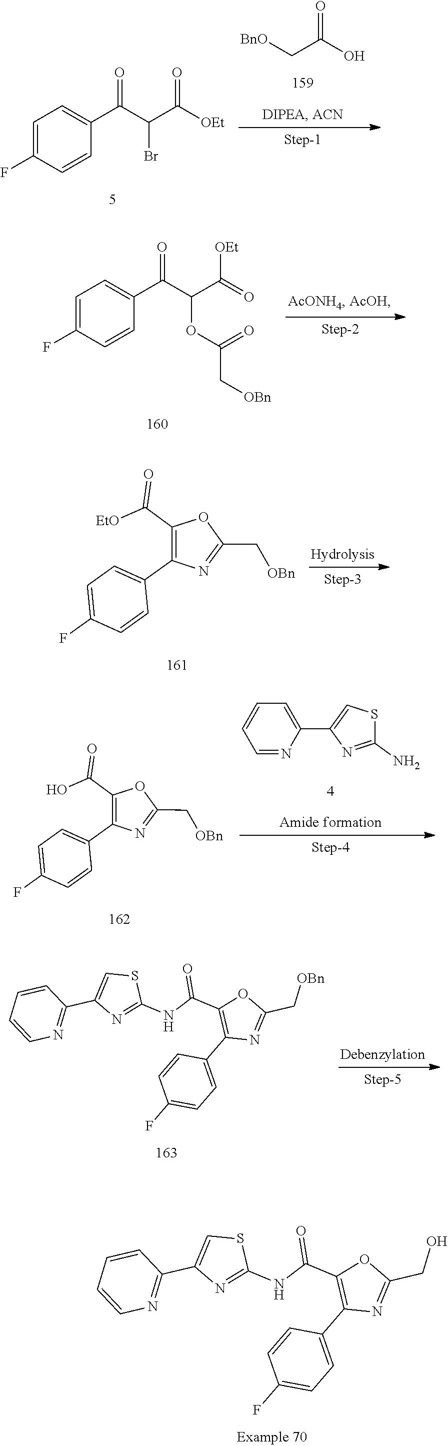

### Step-1: Ethyl 2-(2-(benzyloxy)acetoxy)-3-(4-fluorophenyl)-3-oxopropanoate (160)

To a stirred solution of ethyl 2-bromo-3-(4-fluorophenyl)-3-oxopropanoate (5) (500 mg, 1.73 mmol, 1 eq) in ACN (20 mL), DIPEA (0.58 mL, 3.45 mmol, 2 eq) and benzyloxy acetic acid (159) (574 mg, 3.45 mmol, 2 eq) were added at RT. The reaction mixture was refluxed for 12 h. The progress of the reaction was monitored by TLC (M.Ph: 20% EtOAc in n-hexane). After completion of reaction, the reaction mixture was concentrated under reduced pressure. The crude compound was purified by combi-flash (0-10% EtOAc in n-hexane) to afford 160 (320 mg, 49%) as a viscous mass. ¹H NMR (400 MHz, DMSO-d₆) δ 8.15-8.12 (m, 2H), 7.44 (t, J=8.8 Hz, 2H), 7.35-7.32 (m, 5H), 6.71 (s, 1H), 4.55 (s, 2H), 4.35 (s, 2H), 4.20 (q, J=6.8 Hz, 2H), 1.13 (t, J=6.8 Hz, 3H); LC-MS: m/z 397.0 [M+H]⁺.

### Step-2: Ethyl 2-((benzyloxy)methyl)-4-(4-fluorophenyl)oxazole-5-carboxylate (161)

To a stirred solution of ethyl 2-(2-(benzyloxy)acetoxy)-3-(4-fluorophenyl)-3-oxopropanoate (160) (320 mg, 0.85 mmol, 1 eq) in AcOH (5 mL), ammonium acetate (131 mg, 1.70 mmol, 2 eq) was added at 0° C. The reaction mixture was stirred at 110° C. for 6 h. The progress of the reaction was monitored by TLC (M.Ph: 30% EtOAc in n-hexane). After completion of reaction, the reaction mixture was concentrated to dryness under reduced pressure.

The resulting residue was diluted with water and extracted with diethyl ether (2×75 mL). The combined organic layers was washed with brine, dried over anhydrous Na₂SO₄ and concentrated under reduced pressure and triturated by n-pentane, filtered to afford 161 (120 mg, 42%) as a low melting brown solid. ¹H NMR (400 MHz, DMSO-d₆) δ 8.10-8.06 (m, 2H), 7.38-7.31 (m, 7H), 4.74 (s, 2H), 4.36 (q, J=7.2 Hz, 2H), 1.31 (t, J=7.2 Hz, 3H); LC-MS: m/z 356.20 [M+H]⁺.

### Step-3: 2-((Benzyloxy)methyl)-4-(4-fluorophenyl)oxazole-5-carboxylic acid (162)

To a stirred mixture of ethyl 2-((benzyloxy)methyl)-4-(4-fluorophenyl)oxazole-5-carboxylate (161) (120 mg, 0.34 mmol, 1 eq) in THF:H₂O (2:1, 7.5 mL), LiOH·H₂O (28 mg, 0.67 mmol, 2.0 eq) was added at RT. The reaction mixture was stirred at RT for 12 h. The progress of the reaction was monitored by TLC (M.Ph: 20% EtOAc in n-hexane). The reaction mixture was concentrated under reduced pressure. The resulting residue was diluted with water (50 mL). The aqueous layer was acidified with 6N HCl and extracted with EtOAc (2×75 mL). The combined organic layers was washed with brine, dried over sodium sulfate, filtered and concentrated to dryness to afford titled compound 162 (86 mg, 78%) as an off-white solid. ¹H NMR (400 MHz, DMSO-d₆) δ 8.02-7.99 (m, 2H), 7.34-7.24 (m, 7H), 4.67 (s, 2H), 4.59 (s, 2H); LC-MS: m/z 327.95 [M+H]⁺.

### Step-4: 2-((Benzyloxy)methyl)-4-(4-fluorophenyl)-N-(4-(pyridin-2-yl)thiazol-2-yl)oxazole-5-carboxamide (163)

In line with General Procedure A, 2-((benzyloxy)methyl)-4-(4-fluorophenyl)oxazole-5-carboxylic acid (5) (80 mg, 0.24 mmol, 1 eq) was coupled with 4-(pyridin-2-yl)thiazol-2-amine (6) (47 mg, 0.26 mmol, 1.1 eq) to afford 163 (40 mg, 34%) as an off-white solid. ¹H NMR (400 MHz, DMSO-d₆) δ 8.56 (bs, 1H), 8.17 (bs, 2H), 8.05 (d, J=7.6 Hz, 1H) 7.88 (bs, 2H), 7.37-7.30 (m, 8H), 4.76 (s, 2H), 4.68 (s, 2H); LC-MS: m/z 487.15 [M+H]⁺; HPLC: 97.9%.

### Step-5: Example 70

To a stirred solution of 2-((benzyloxy)methyl)-4-(4-fluorophenyl)-N-(4-(pyridin-2-yl)thiazol-2-yl)oxazole-5-carboxamid (163) (20 mg, 0.04 mmol, 1 eq) in DCM (5 mL) maintained at 0° C. was added TFA (0.4 mL, 0.41 mmol, 10 eq).The reaction mixture was stirred at 45° C. for 12 h. Progress of the reaction was monitored by TLC. After completion of reaction, the reaction mixture was concentrated under reduced pressure. The crude obtained was triturated using Et₂O and Pentane to afford Example 70 (10 mg, 63%) as an off-white solid. ¹H NMR (400 MHz, DMSO-d₆) δ 12.79 (s, 1H), 8.63-8.61 (m, 1H), 8.25-8.22 (m, 2H), 8.03 (d, J=8.4 Hz, 1H), 7.95-7.89 (m, 2H), 7.41-7.34 (m, 3H), 5.80-5.70 (m, 1H), 4.70 (d, J=5.6 Hz, 2H); LC-MS: m/z 397.20 [M+H]⁺; HPLC: 98.3%.

### Synthesis of Example 71

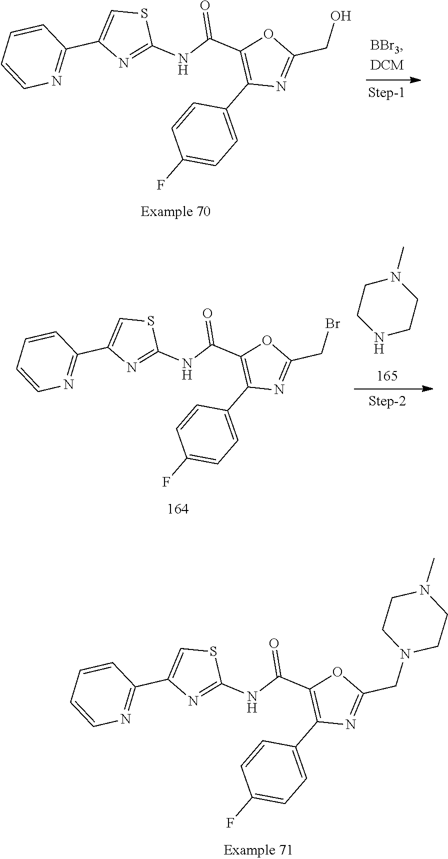

### Step-1:2-(Bromomethyl)-4-(4-fluorophenyl)-N-(4-(pyridin-2-yl)thiazol-2-yl)oxazole-5-carboxamide (164)

To a stirred solution of 4-(4-fluorophenyl)-2-(hydroxymethyl)-N-(4-(pyridin-2-yl)thiazol-2-yl)oxazole-5-carboxamide (Example 70) (100 mg, 0.25 mmol, 1 eq) in DCM (2 mL), BBr₃ (0.04 mL, 0.37 mmol, 1.5 eq) was added and the reaction mixture was stirred at RT for 2 h. The progress of the reaction was monitored by TLC (M.Ph: 20% EtOAc in n-hexane). After completion of reaction, the reaction mixture was diluted with ice cold water. The aqueous layer was extracted with EtOAc. The organic layer was washed thoroughly with cold water and brine, dried over anhydrous Na₂SO₄ and concentrated under reduced pressure. The crude compound was purified by combi-flash (30% EtOAc/hexane) to afford 164 (22 mg, 19%) as an off-white solid. ¹H NMR (400 MHz, DMSO-d₆) δ 13.34 (s, 1H), 8.64 (bs, 1H), 8.24 (d, J=6.4 Hz, 1H), 8.06-7.99 (m, 3H), 7.40-7.36 (m, 3H), 5.01 (s, 1H), 4.69 (s, 1H); LC-MS: m/z 459.10 [M+H]⁺.

### Step-2: Example 71

To a stirred solution of 2-(bromomethyl)-4-(4-fluorophenyl)-N-(4-(pyridin-2-yl)thiazol-2-yl)oxazole-5-carboxamide (164) (25 mg, 0.054 mmol, 1 eq) in ACN (2 mL), N-methyl piperazine (165) (6.5 mg, 0.065 mmol, 1.2 eq) and K₂CO₃ (11.2 mg, 0.081 mmol, 1.5 eq) were added and the reaction mixture was stirred at RT for 16 h. The progress of the reaction was monitored by TLC (M.Ph: 30% EtOAc in n-hexane). After completion of reaction, the reaction mixture was concentrated to dryness under reduced pressure. The resulting residue was diluted with water and extracted with EtOAc (2×25 mL). The combined organic layers was washed with brine, dried over anhydrous Na₂SO₄ and concentrated under reduced pressure and The crude compound was purified by combi-flash (30% EtOAc/hexane) to afford Example 71 (13 mg, 50%) as an off-white solid. ¹H NMR (400 MHz, DMSO-d₆) δ 12.56 (s, 1H), 8.62 (bs, 1H), 8.27-8.24 (m, 2H), 8.03 (d, J=8 Hz, 1H), 7.92-7.88 (m, 2H), 7.37-7.33 (m, 3H), 3.81 (s, 2H), 2.62 (bs, 4H), 2.44 (bs, 4H), 2.20 (s, 3H); LC-MS: m/z 479.20 [M+H]⁺, HPLC: 98.7%.

### Synthesis of Example 72

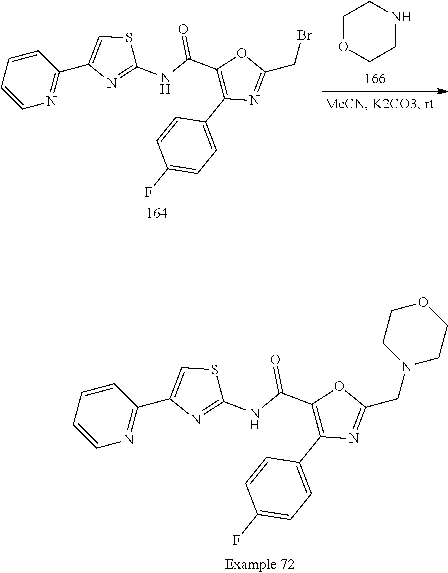

To a stirred solution of 2-(bromomethyl)-4-(4-fluorophenyl)-N-(4-(pyridin-2-yl)thiazol-2-yl)oxazole-5-carboxamide (164) (20 mg, 0.045 mmol, 1 eq) in ACN (2 mL), morpholine (166) (4.55 mg, 0.054 mmol, 1.2 eq) and K₂CO₃ (9 mg, 0.065 mmol, 1.5 eq) were added and the reaction mixture was stirred at RT for 16 h. The progress of the reaction was monitored by TLC (M.Ph: 30% EtOAc in n-hexane). After completion of reaction, the reaction mixture was concentrated to dryness under reduced pressure. The resulting residue was diluted with water and extracted with EtOAc (2×25 mL). The combined organic layers was washed with brine, dried over anhydrous Na₂SO₄ and concentrated under reduced pressure and The crude compound was purified by combi-flash (30% EtOAc/hexane) to afford Example 72 (13 mg, 65%) as an off-white solid. ¹H NMR (400 MHz, DMSO-d₆) δ 12.91 (s, 1H), 8.63 (bs, 1H), 8.26-8.22 (m, 2H), 8.03 (d, J=7.6 Hz, 1H), 7.93-7.89 (m, 2H), 7.38-7.34 (m, 3H), 3.83 (s, 2H), 3.63 (bs, 4H), 2.60 (bs, 4H); LC-MS: m/z 466.10 [M+H]⁺, HPLC: 98.9%.

### Synthesis of Example 73

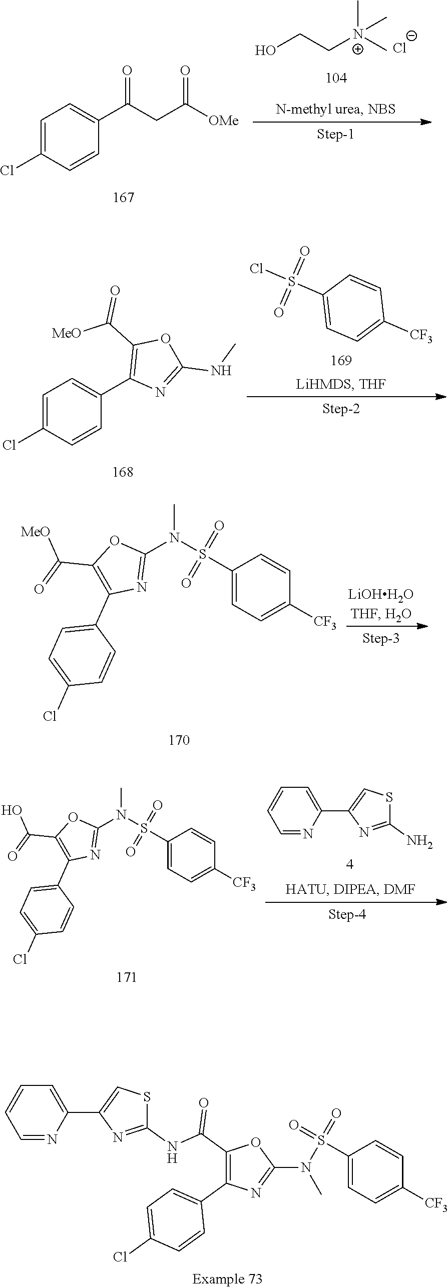

### Step-1: Methyl 4-(4-chlorophenyl)-2-(methylamino)oxazole-5-carboxylate (168)

A mixture of choline chloride (104) (2 g, 14.32 mmol, 1.1 eq) and N-methyl urea (2.1 g, 28.64 mmol, 2 eq) was heated to 60° C. (at this temperature, mixture became homogeneous) and stirred at same temperature for 30 min. To this resulting mixture, methyl 3-(4-chlorophenyl)-3-oxopropanoate (167) (2 g, 12.85 mmol, 1 eq) and NBS (2.2 g, 12.85 mmol, 1 eq) were added at 60° C. and stirred at 80° C. for 24 h. The progress of the reaction was monitored by TLC (M.Ph: 50% EtOAc in n-hexane). After completion of reaction, the reaction mixture was diluted with water and extracted with EtOAc (2×100 mL). The combined organic layers were washed with brine, dried over sodium sulfate, concentrated under reduced pressure. The crude compound was purified by silica gel column chromatography (0-30% EtOAc in n-hexane) to afford 168 (310 mg, 9%) as an off-white solid. LC-MS: m/z 267.0 [M+H]⁺.

### Step-2: Methyl 4-(4-chlorophenyl)-2-((N-methyl-4-(trifluoromethyl)phenyl)sulfonamido)oxazole-5-carboxylate (170)

To a stirred solution of methyl 4-(4-chlorophenyl)-2-(methylamino)oxazole-5-carboxylate (168) (150 mg, 0.56 mmol, 1 eq) in THE (10 mL), LiHMDS (1M in THF, 1.12 mL, 1.127 mmol, 2 eq) was added at −78° C. After stirring for 5 min at same temperature, 4-(trifluoromethyl)benzenesulfonyl chloride (169) (266 mg, 0.84 mmol, 1.2 eq) was added slowly at −78° C. and the reaction mixture was stirred at RT for 12 h. The progress of the reaction was monitored by TLC (M.Ph: 50% EtOAc in n-hexane). After completion of reaction, the reaction mixture was quenched with sat. NH₄Cl solution. The mixture was extracted with EtOAc (2×100 mL). The combined organic layers was washed with brine, dried over anhydrous Na₂SO₄ and concentrated under reduced pressure and the crude compound was purified by silica gel column chromatography (0-20% EtOAc in n-hexane) to afford 170 (210 mg, 79%) as a white solid. LC-MS: m/z 474.95 [M+H]⁺.

### Step-3: 4-(4-Chlorophenyl)-2-((N-methyl-4-(trifluoromethyl)phenyl)sulfonamido)oxazole-5-carboxylic acid (171)

To a stirred mixture of methyl 4-(4-chlorophenyl)-2-((N-methyl-4-(trifluoromethyl)phenyl)sulfonamido)oxazole-5-carboxylate (170) (200 mg, 0.42 mmol, 1 eqv) in THF:water (1.5:1, 10 mL), LiOH·H₂O (35 mg, 0.84 mmol, 2 eq) was added at RT. The reaction mixture was stirred at RT for 2 h. The progress of the reaction was monitored by TLC (M.Ph: 50% EtOAc in n-hexane). After completion of reaction, the solvent was removed under reduced pressure. The residue was diluted with water and adjusted pH to 2-3 with 1N HCl. The solid so obtained was filtered and dried to afford 171 (158 mg, 82%) as an off-white solid. LC-MS: m/z 460.95 [M+H]⁺.

### Step-4: Example 73

In line with the General procedure A, 4-(4-chlorophenyl)-2-((N-methyl-4-(trifluoromethyl)phenyl)sulfonamido)oxazole-5-carboxylic acid (171) (150 mg, 0.32 mmol, 1 eq) was coupled with 4-(pyridin-2-yl)thiazol-2-amine (4) (69 mg, 0.39 mmol, 1.2 eq) to afford Example 73 (30 mg, 15%) as an off-white solid. ¹H NMR (400 MHz, DMSO-d₆) δ 12.99 (s, 1H), 8.61 (bs, 1H), 8.39 (d, J=8.4 Hz, 2H), 8.20 (d, J=8.8 Hz, 2H), 8.13 (d, J=8.8 Hz, 2H), 8.03 (d, J=8.8 Hz, 1H), 7.92-7.88 (m, 2H), 7.62 (d, J=8.0 Hz, 2H), 7.36 (t, J=8.0 Hz, 1H), 3.73 (s, 3H); LC-MS: m/z 620.10 [M+H]⁺; HPLC: 97.0%.

### Synthesis of Example 74

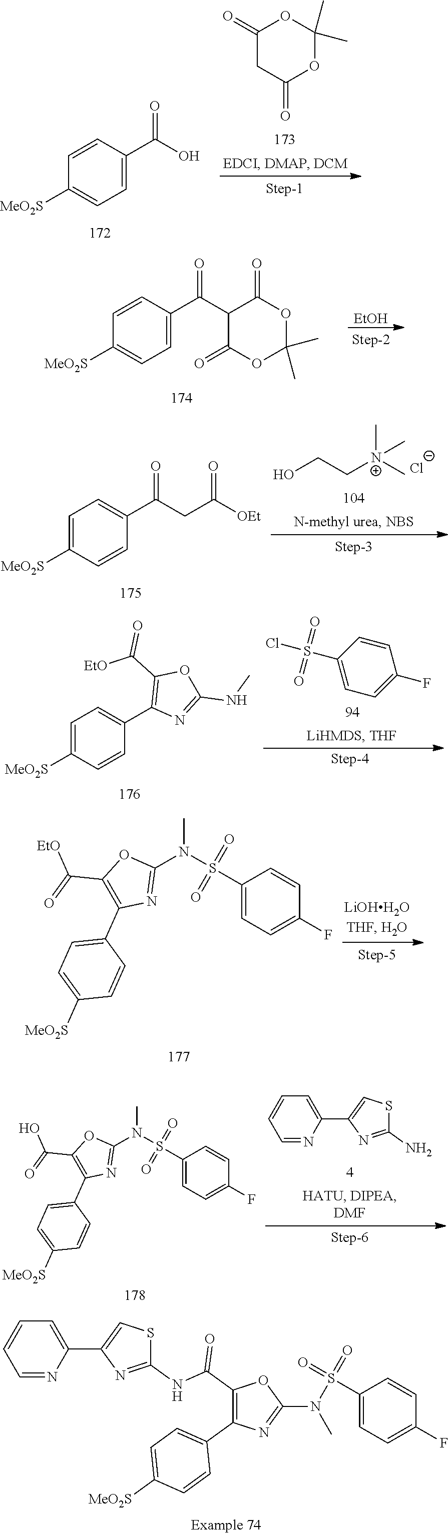

### Step-1: 2,2-Dimethyl-5-(4-(methylsulfonyl)benzoyl)-1,3-dioxane-4,6-dione (174)

To a stirred solution of 4-(methylsulfonyl)benzoic acid (172) (5 g, 24.9 mmol, 1 eq) in DCM (100 mL), Meldrum's acid (173) (4.32 g, 29.88 mmol, 1.2 eq), DMAP (4.58 g, 37.35 mmol, 1.5 eq) and EDCI.HCl (6.24 g, 32.5 mmol, 1.3 eq) was added slowly at RT. The reaction mixture was stirred at RT for 12 h. The progress of the reaction was monitored by TLC (M.Ph: 25% EtOAc in n-hexane). The reaction mass was diluted with DCM (100 mL) and washed with 1N HCl (50 mL). The organic layer was washed with brine, dried over sodium sulfate, filtered and concentrated to dryness to afford 174 (4.2 g, 51%) as a colorless liquid. ¹H NMR (400 MHz, DMSO-d₆) δ 7.94 (d, J=7.6, Hz, 2H), 7.70 (d, J=7.6 Hz, 2H), 4.02 (s, 3H), 1.71 (s, 6H).

### Step-2: Ethyl 3-(4-(methylsulfonyl)phenyl)-3-oxopropanoate (175)

A solution of methyl 2,2-dimethyl-5-(4-(methylsulfonyl)benzoyl)-1,3-dioxane-4,6-dione (174) (4.1 g, 12.5 mmol, 1 eq) and EtOH (50 mL) was refluxed for 12 h. The progress of the reaction was monitored by TLC (M.Ph: 5% MeOH in DCM). After completion of reaction, the reaction mixture was concentrated to dryness under reduced pressure. The residue was purified through silica gel column chromatography (elution: 0-5% EtOAc in n-hexane) to afford 175 (1.17 g, 34%) as an off-white solid. ¹H NMR (400 MHz, DMSO-d₆) δ 8.19 (d, J=8.4 Hz, 2H), 8.10 (d, J=8.4 Hz, 2H), 4.28 (s, 2H), 4.19 (q, J=6.8 Hz. 2H), 3.27 (s, 3H), 1.19 (t, J=6.8 Hz, 3H).

### Step-3: Ethyl 2-(methylamino)-4-(4-(methylsulfonyl)phenyl)oxazole-5-carboxylate (176)

A mixture of choline chloride (104) (1.08 g, 77.07 mmol, 1.9 eq) and N-methyl urea (1.1 g, 150.74 mmol, 3.7 eq) was heated to 60° C. (at this temperature, mixture became homogeneous) and stirred at same temperature for 30 min. To this resulting mixture, ethyl 3-(4-(methylsulfonyl)phenyl)-3-oxopropanoate (175) (1.1 g, 40.74 mmol, 1 eq) and NBS (650 mg, 36.66 mmol, 0.9 eq) were added at 60° C. and stirred at 80° C. for 24 h. The progress of the reaction was monitored by TLC (M.Ph: 50% EtOAc in n-hexane). After completion of reaction, the reaction mixture was diluted with water and extracted with EtOAc (2×100 mL). The combined organic layers was washed with brine, dried over sodium sulfate, concentrated under reduced pressure. The crude compound was purified by silica gel column chromatography (0-30% EtOAc in n-hexane) to afford 176 (400 mg, 30%) as an off-white solid. ¹H NMR (400 MHz, DMSO-d₆) δ 8.13 (d, J=8.0 Hz, 2H), 7.95 (d, J=8.4 Hz, 2H), 4.20 (q, J=6.8 Hz. 2H), 3.19 (s, 3H), 2.85 (s, 3H), 1.24 (t, J=6.8 Hz, 3H); LC-MS: m/z 324.6 [M+H]⁺.

### Step-4: Ethyl 2-((4-fluoro-N-methylphenyl)sulfonamido)-4-(4-(methylsulfonyl)phenyl)oxazole-5-carboxylate (177)

To a stirred solution of ethyl 2-(methylamino)-4-(4-(methylsulfonyl)phenyl)oxazole-5-carboxylate (176) (400 mg, 1.23 mmol, 1 eq) in THE (10 mL), LiHMDS (1M in THF, 3.08 mL, 3.07 mmol, 2.5 eq) was added at −78° C. After stirring for 5 min at same temperature, 4-fluorobenzenesulfonyl chloride (94) (359 mg, 1.84 mmol, 1.5 eq) was added slowly at −78° C. and the reaction mixture was stirred at RT for 12 h. The progress of the reaction was monitored by TLC (M.Ph: 50% EtOAc in n-hexane). After completion of reaction, the reaction mixture was quenched with sat. NH₄Cl solution. The mixture was extracted with EtOAc (2×100 mL). The combined organic layers was washed with brine, dried over anhydrous Na₂SO₄ and concentrated under reduced pressure and the crude compound was purified by silica gel column chromatography (0-20% EtOAc in n-hexane) to afford 177 (180 mg, 30%) as a white solid. ¹H NMR (400 MHz, DMSO-d₆) δ 8.19-8.16 (m, 4H), 8.04 (d, J=8.4 Hz, 2H), 7.59 (t, J=8.8 Hz, 2H), 4.35 (q, J=6.8 Hz. 2H), 3.53 (s, 3H), 3.26 (s, 3H), 1.32 (t, J=6.8 Hz, 3H); LC-MS: m/z 482.82 [M+H]⁺.

### Step-5: 2-((4-Fluoro-N-methylphenyl)sulfonamido)-4-(4-(methylsulfonyl)phenyl)oxazole-5-carboxylic acid (178)

To a stirred mixture of ethyl 2-((4-fluoro-N-methylphenyl)sulfonamido)-4-(4-(methylsulfonyl)phenyl)oxazole-5-carboxylate (177) (170 mg, 0.35 mmol, 1 eq) in THF:water (1.5:1, 5 mL), LiOH·H₂O (23 mg, 0.51 mmol, 1.5 eq) was added at RT. The reaction mixture was stirred at RT for 2 h. The progress of the reaction was monitored by TLC (M.Ph: 50% EtOAc in n-hexane). After completion of reaction, the solvent was removed under reduced pressure. The residue was diluted with water and adjusted pH upto 2-3 with 1N HCl. The solid so obtained was filtered and dried to afford 178 (120 mg, 75%) as an off-white solid. ¹H NMR (400 MHz, DMSO-d₆) δ 13.88 (bs, 1H), 8.22-8.14 (m, 4H), 8.02 (d, J=8.0 Hz, 2H), 7.57 (t, J=8.8 Hz, 2H), 3.49 (s, 3H), 3.26 (s, 3H); LC-MS: m/z 454.9 [M+H]⁺.

### Step-6: Example 74

In line with the General Procedure A, 2-((4-fluoro-N-methylphenyl)sulfonamido)-4-(4-(methylsulfonyl)phenyl)oxazole-5-carboxylic acid (178) (110 mg, 0.24 mmol, 1 eq) was coupled with 4-(pyridin-2-yl)thiazol-2-amine (4) (38 mg, 0.24 mmol, 1 eq) to afford Example 74 (30 mg, 20%) as an off-white solid. ¹H NMR (400 MHz, DMSO-d₆) δ 12.95 (s, 1H), 8.48 (bs, 1H), 8.30 (d, J=8.4 Hz, 2H), 8.22-8.21 (m, 2H), 8.05-8.02 (m, 3H), 7.88-7.85 (m, 2H), 7.53 (t, J=7.0 Hz, 2H), 7.37 (bs, 1H), 3.64 (s, 3H), 3.23 (s, 3H); LC-MS: m/z 613.90 [M+H]⁺; HPLC: 96.9%.

### Synthesis of Example 75

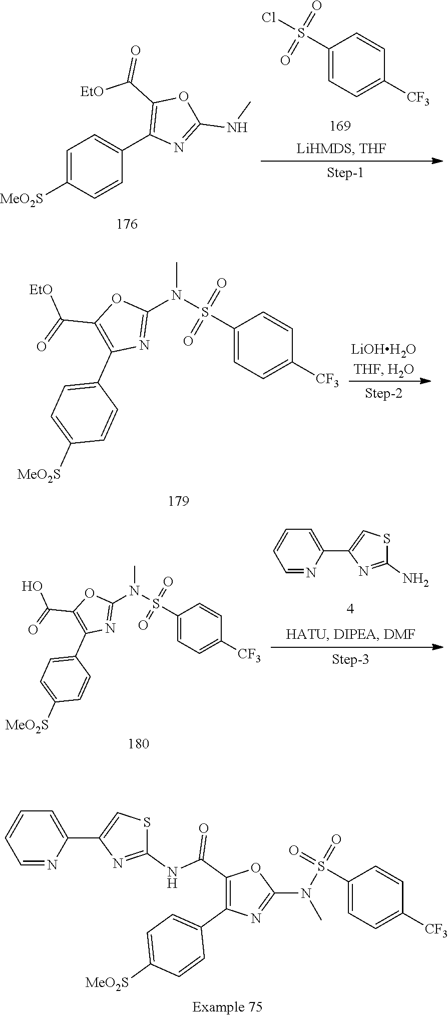

### Step-1: Ethyl 2-((N-methyl-4-(trifluoromethyl)phenyl)sulfonamido)-4-(4-(methylsulfonyl)phenyl)oxazole-5-carboxylate (179)

To a stirred solution of ethyl 2-(methylamino)-4-(4-(methylsulfonyl)phenyl)oxazole-5-carboxylate (176) (200 mg, 0.64 mmol, 1 eq) in THF (10 mL), LiHMDS (1M in THF, 1.2 mL, 1.29 mmol, 2 eq) was added at −78° C. After stirring for 5 min at same temperature, 4-trifluoromethylbenzenesulfonyl chloride (169) (189 mg, 0.77 mmol, 1.2 eq) was added slowly at −78° C. and the reaction mixture was stirred at RT for 12 h. The progress of the reaction was monitored by TLC (M.Ph: 50% EtOAc in n-hexane). After completion of reaction, the reaction mixture was quenched with sat. NH₄Cl solution. The mixture was extracted with EtOAc (2×100 mL). The combined organic layers was washed with brine, dried over anhydrous Na₂SO₄ and concentrated under reduced pressure and the crude compound was purified by silica gel column chromatography (0-20% EtOAc in n-hexane) to afford 179 (150 mg, 22%) as a white solid. ¹H NMR (400 MHz, DMSO-d₆) δ 8.35 (d, J=8.4 Hz, 2H), 8.22 (d, J=8.4 Hz, 2H), 8.14 (d, J=8.4 Hz, 2H), 8.06 (d, J=8.0 Hz, 2H), 4.37 (q, J=7.2 Hz. 2H), 3.58 (s, 3H), 3.28 (s, 3H), 1.33 (t, J=7.6 Hz, 3H); LC-MS: m/z 532.9 [M+H]⁺.

### Step-2: 2-((N-methyl-4-(trifluoromethyl)phenyl)sulfonamido)-4-(4-(methylsulfonyl)phenyl)oxazole-5-carboxylic acid (180)

To a stirred mixture of ethyl 2-((N-methyl-4-(trifluoromethyl)phenyl)sulfonamido)-4-(4-(methylsulfonyl)phenyl)oxazole-5-carboxylate (179) (150 mg, 0.28 mmol, 1 eqv) in THF:water (1.5:1, 5 mL), LiOH·H₂O (29 mg, 0.70 mmol, 2.5 eq) was added at RT. The reaction mixture was stirred at RT for 2 h. The progress of the reaction was monitored by TLC (M.Ph: 50% EtOAc in n-hexane). After completion of reaction, the solvent was removed under reduced pressure. The residue was diluted with water and adjusted to pH 2-3 with 1N HCl. The solid so obtained was filtered and dried to afford 180 (105 mg, 74%) as an off-white solid. ¹H NMR (400 MHz, DMSO-d₆) δ 13.88 (bs, 1H), 8.22-8.14 (m, 4H), 8.02 (d, J=8.0 Hz, 2H), 7.57 (t, J=8.8 Hz, 2H), 3.49 (s, 3H), 3.26 (s, 3H); LC-MS: m/z 505.1 [M+H]⁺.

### Step-3: Example 75

To a stirred solution of 2-((N-methyl-4-(trifluoromethyl)phenyl)sulfonamido)-4-(4-(methylsulfonyl)phenyl)oxazole-5-carboxylic acid (180) (100 mg, 0.19 mmol, 1 eq) in DMF (10 mL), DIPEA (0.1 mL, 0.59 mmol, 3 eq) and HATU (150 mg, 0.38 mmol, 2 eq) were added and the reaction mixture was stirred for 5 min. To the resulting reaction mixture, 4-(pyridin-2-yl)thiazol-2-amine (4) (52 mg, 0.29 mmol, 1.5 eq) was added and the reaction mixture was stirred at RT for 10 h. The progress of the reaction was monitored by TLC (M.Ph: 5% MeOH in DCM). After completion of reaction, the reaction mixture was diluted with ice cold water. The aqueous layer was extracted with EtOAc (2×50 mL). The organic layer was washed thoroughly with cold water and brine, dried over anhydrous Na₂SO₄ and concentrated under reduced pressure. The crude compound was purified by combi-flash (30% EtOAc/hexane) to afford Example 75 (27 mg, 21%) as an off-white solid. ¹H NMR (400 MHz, DMSO-d₆) δ 12.99 (s, 1H), 8.62 (bs, 1H), 8.41 (d, J=8.4 Hz, 2H), 8.34 (d, J=8.8 Hz, 2H), 8.16 (d, J=8.8 Hz, 2H), 8.10 (d, J=8.8 Hz, 2H), 8.03 (d, J=8.0 Hz, 1H), 7.93-7.87 (m, 2H), 7.37-7.34 (m, 1H), 3.74 (s, 3H), 3.29 (s, 3H); LC-MS: m/z 664.16 [M+H]⁺; HPLC: 98.0%.

### Synthesis of Example 76

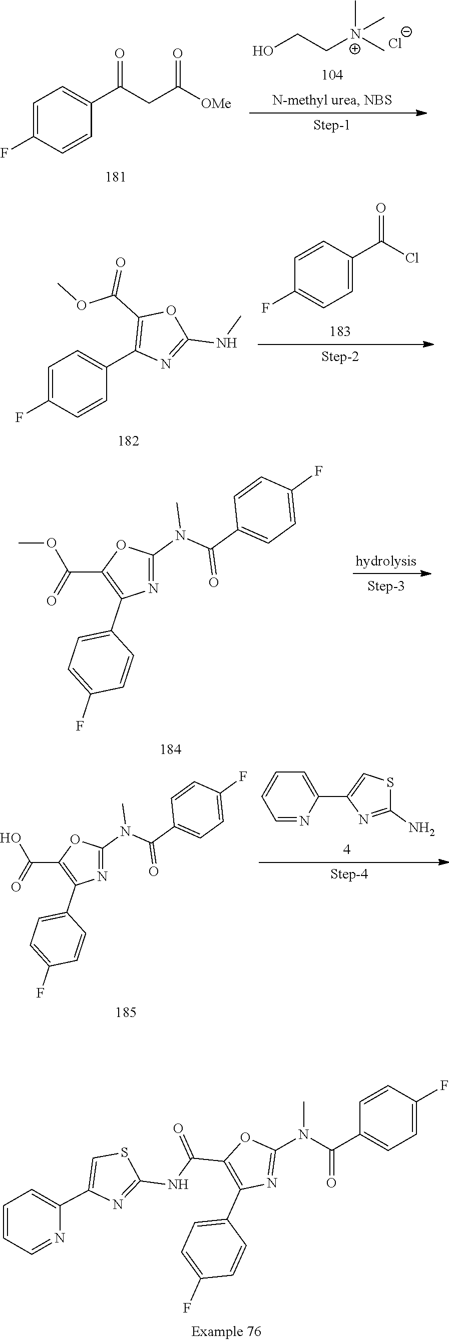

### Step-1: Methyl 4-(4-fluorophenyl)-2-(methylamino)oxazole-5-carboxylate (182)

A mixture of choline chloride (104) (6.8 g, 48.7 mol, 1.9 eq) and N-methyl urea (7.1 g, 95.94 mol, 3.7 eq) was heated to 60° C. (at this temperature, mixture became homogeneous) and stirred at same temperature for 30 min. To this resulting mixture, methyl 3-(4-fluorophenyl)-3-oxopropanoate (181) (5 g, 25.51 mol, 1 eq) and NBS were added at 60° C. and stirred at 80° C. for 48 h. The progress of the reaction was monitored by TLC (M.Ph: 20% EtOAc in n-hexane). After completion of reaction, the reaction mixture was diluted with water and extracted with EtOAc (2×250 mL). The combined organic layers was washed with brine, dried over sodium sulfate, concentrated under reduced pressure. The crude compound was purified by silica gel column chromatography (0-10% EtOAc in n-hexane) to afford 182 (1.5 g, 24%) as a white solid. ¹H NMR (400 MHz, DMSO-d₆) δ 8.12-8.09 (m, 2H), 7.29 (t, J=8.8 Hz, 2H), 3.75 (s, 3H), 2.87 (s, 3H); LC-MS: m/z 251.4 [M+H]⁺.

### Step-2: Methyl 2-(4-fluoro-N-methylbenzamido)-4-(4-fluorophenyl)oxazole-5-carboxylate (184)

To a stirred solution of methyl 4-(4-fluorophenyl)-2-(methylamino)oxazole-5-carboxylate (182) (700 mg, 2.8 mmol, 1 eq) in EDC (10 mL), DIPEA (1.4 mL, 8.4 mmol, 3 eq) was added at 0° C. After stirring for 10 min at same temperature, 4-fluoro benzoylchloride (183) (0.5 mL, 3.0 mmol, 1.1 eq) was added and the reaction mixture was stirred at reflux temperature for 8 h. The progress of the reaction was monitored by TLC (M.Ph: 30% EtOAc in n-hexane). After completion of reaction, the reaction mixture was diluted with water and extracted with DCM (2×200 mL). The combined organic layers was washed with brine, dried over anhydrous Na₂SO₄ and concentrated under reduced pressure and the crude compound was purified by silica gel column chromatography (0-10% EtOAc in n-hexane) to afford 184 (640 mg, 49%) as a white solid. ¹H NMR (400 MHz, DMSO-d₆) δ 8.02-7.98 (m, 1H), 7.95-7.981 (m, 2H), 7.62-7.58 (m, 2H), 7.34-7.25 (m, 3H), 3.73 (s, 3H), 3.51 (s, 3H); LC-MS: m/z 373.4 [M+H]⁺.

### Step-3: 2-(4-Fluoro-N-methylbenzamido)-4-(4-fluorophenyl)oxazole-5-carboxylic acid (185)

To a stirred mixture of methyl 2-(4-fluoro-N-methylbenzamido)-4-(4-fluorophenyl)oxazole-5-carboxylate (184) (300 mg, 0.80 mmol, 1 eq) in DCM (10 mL), BBr₃ (3.2 mL, 1.6 mmol, 2.0 eq) was added at 0° C. The reaction mixture was stirred at RT for 48 h.

The progress of the reaction was monitored by TLC (M.Ph: 70% EtOAc in n-hexane). After completion of reaction, the excess of BBr₃ was decomposed by adding few drops of MeOH.

Then the reaction mixture was diluted with water and extracted with DCM (2×100 mL). The combined organic layers were washed with water, dried over sodium sulfate, filtered and concentrated to dryness to afford 185 (150 mg, 52%) as an off-white solid. ¹H NMR (400 MHz, DMSO-d₆) δ 7.83-7.80 (m, 2H), 7.52-7.49 (m, 2H), 7.20 (t, J=8.4 Hz, 4H), 3.45 (s, 3H); LC-MS: m/z 356.9 [M+H]⁻.

### Step-4: Example 76

In line with the General Procedure A, 2-(4-fluoro-N-methylbenzamido)-4-(4-fluorophenyl)oxazole-5-carboxylic acid (185) (140 mg, 0.39 mmol, 1 eq) was coupled with 4-(pyridin-2-yl)thiazol-2-amine (4) (76 mg, 0.43 mmol, 1.1 eq) to afford Example 76 (24 mg, 12%) as an off-white solid. ¹H NMR (400 MHz, DMSO-d₆) δ 12.71 (s, 1H), 8.56 (bs, 1H), 8.06 (d, J=7.6 Hz, 1H), 7.92 (t, J=7.6 Hz, 2H), 7.84 (s, 1H), 7.62 (t, J=8.4 Hz, 3H), 7.36 (t, J=6.0 Hz, 1H), 7.24 (t, J=8.4 Hz, 4H), 3.57 (s, 3H); LC-MS: m/z 518.05 [M+H]⁺; HPLC: 99.7%.

### Synthesis of Example 77

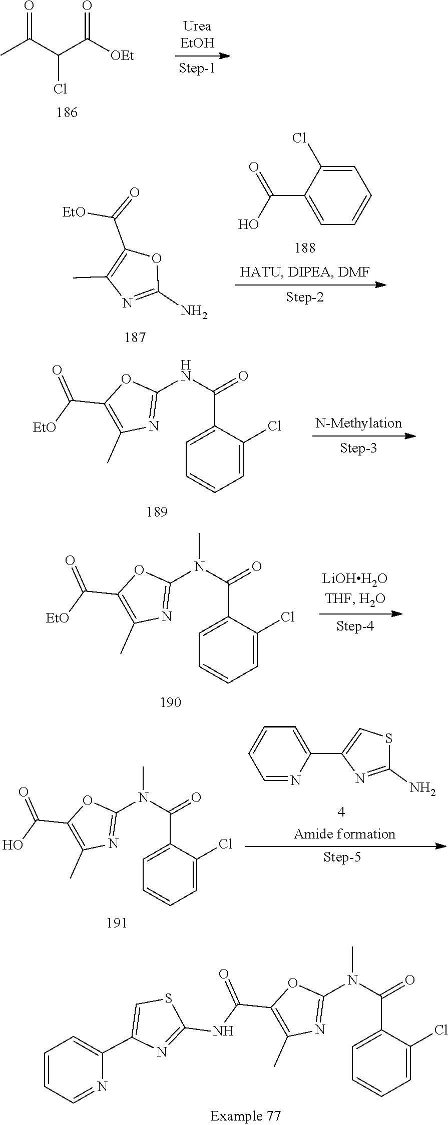

### Step-1: Ethyl 2-amino-4-methyloxazole-5-carboxylate (187)

To a stirred mixture of ethyl 2-chloro-3-oxobutanoate (186) (7 g, 42.68 mmol, 1 eq) in EtOH (35 mL), urea (7.6 g, 128.04 mmol, 3 eq) was added at RT. The mixture was stirred at 95° C. for 12 h. The progress of the reaction was monitored by TLC (M.Ph: 5% MeOH in DCM).

The obtained solid was filtered and the wet cake was diluted with water (75 mL) and extracted with EtOAc (2×150 mL). The combined organic extract was washed with brine, dried over sodium sulfate, filtered and concentrated to dryness to afford 187 (3.5 g, 48%) as white solid. ¹H NMR (400 MHz, DMSO-d₆) δ 8.00 (s, 2H), 4.29 (q, J=7.2 Hz, 2H), 1.27 (t, J=7.2 Hz, 3H); LC-MS: m/z 170.95 [M+H]⁺.

### Step-2: Ethyl 2-(2-chlorobenzamido)-4-methyloxazole-5-carboxylate (189)

To a stirred mixture of ethyl 2-amino-4-methyloxazole-5-carboxylate (187) (1.26 g, 0.47 mmol, 1 eq) in DMF (20 mL), HATU (5.36 g, 14.82 mmol, 2 eq) and DIPEA (3.5 mL, 22.23 mmol, 3.0 eq) were added at RT. The mixture was stirred at same temperature for 30 min.

Then 2-chlorobenzoic acid (188) (1.3 g, 8.89 mmol, 1.2 eq) was added into the reaction mixture and allowed to stirred at RT for 12 h. The progress of the reaction was monitored by TLC (M.Ph: 5% MeOH in DCM). The reaction mixture diluted with water (75 mL) and extracted with EtOAc (2×150 mL). The combined organic extract was washed with brine, dried over sodium sulfate, filtered and concentrated to dryness. The crude was purified through silica gel column chromatography (elution: 10-40% EtOAc in n-hexane) to afford 189 (1.1 g, 48%) as an off-white solid. ¹H NMR (400 MHz, DMSO-d₆) δ 7.60-7.56 (m, 1H), 7.54-7.50 (m, 2H), 7,45-7.42 (m, 2H), 4.29 (q, J=7.2 Hz, 2H), 2.67 (s, 3H), 1.27 (t, J=7.2 Hz, 3H); LC-MS: m/z 309.1 [M+H]⁺.

### Step-3: Ethyl 2-(2-chloro-N-methylbenzamido)-4-methyloxazole-5-carboxylate (190)

To a stirred solution of ethyl 2-(2-chlorobenzamido)-4-methyloxazole-5-carboxylate (189) (1.0 g, 3.24 mmol, 1 eq) in ACN (20 mL), K₂CO₃ (1.12 g, 8.11 mmol, 2.5 eq) and Mel (0.62 mL, 9.74 mmol, 3 eq) were added at 0° C. The reaction mixture was stirred at RT for 12 h. The progress of the reaction was monitored by TLC (M.Ph: 20% EtOAc in n-hexane). After completion of reaction, the reaction mixture was filtered through hyflo by giving EtOAc wash. The filtrate was concentrated to dryness. The crude was diluted with water and extracted with diethyl ether (2×150 mL). The organic mass was concentrated to dryness to afford 190 (0.8 g, 77%) as a brown viscous oil. LC-MS: m/z 322.95 [M+H]⁺.

### Step-4: 2-(2-Chloro-N-methylbenzamido)-4-methyloxazole-5-carboxylic acid (191)

To a stirred solution of ethyl 2-(2-chloro-N-methylbenzamido)-4-methyloxazole-5-carboxylate (190) (500 mg, 1.55 mmol, 1 eq) in DCM (5 mL), BBr₃ (1 M in DCM, 15.5 mL, 15.52 mmol, 10 eq) was added at 0° C. The reaction mixture was stirred at RT for 12 h. The progress of the reaction was monitored by TLC (M.Ph: 30% EtOAc in n-hexane). After completion of reaction, the reaction mixture was quenched with MeOH (1 mL) followed by ice chilled water. The mixture was extracted with DCM (2×50 mL). The layers were separated and the combined organic extract was concentered to dryness. The residue was triturated with diethyl ether and filtered to afford 191 (380 mg, 83%) as an off-white solid. ¹H NMR (400 MHz, DMSO-d₆) δ 13.33 (s, 1H), 7.46-7.41 (m, 3H), 7.40-7.37 (m, 1H), 3.45 (s, 3H), 2.20 (s, 3H); LC-MS: m/z 294.9 [M+H]⁺.

### Step-5: Example 77

In line with the General procedure A, 2-(2-chloro-N-methylbenzamido)-4-methyloxazole-5-carboxylic acid (191) (300 mg, 1.02 mmol, 1 eq) was coupled with 4-(pyridin-2-yl)thiazol-2-amine (4) (216 mg, 1.22 mmol, 1.2 eqv) to afford Example 77 (130 mg, 28%) as an off-white solid. ¹H NMR (400 MHz, DMSO-d₆) δ 12.54 (s, 1H), 8.61 (d, J=4.4 Hz, 1H), 8.04 (d, J=8.4 Hz, 1H), 7.93-7.89 (m, 2H), 7.51-7.48 (m, 3H), 7.43-7.40 (m, 1H), 7.37 (t, J=8.4 Hz, 1H), 3.61 (s, 3H), 2.18 (s, 3H); LC-MS: m/z 454.00 [M+H]⁺; HPLC: 98.7%.

### Synthesis of Example 78

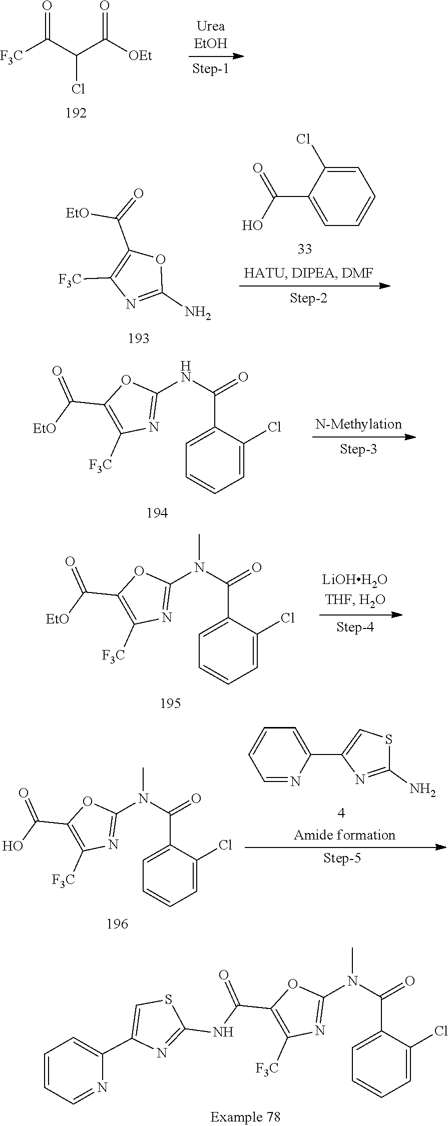

### Step-1: Ethyl 2-amino-4-(trifluoromethyl)oxazole-5-carboxylate (193)

To a stirred mixture of ethyl 2-chloro-4,4,4-trifluoro-3-oxobutanoate (192) (5 g, 23.14 mmol, 1 eq) in EtOH (25 mL), urea (4.1 g, 69.44 mmol, 3 eq) was added at RT. The mixture was stirred at 95° C. for 12 h. The progress of the reaction was monitored by TLC (M.Ph: 5% MeOH in DCM). The obtained solid was filtered and the wet cake was diluted with water (75 mL) and extracted with EtOAc (2×150 mL). The combined organic extract was washed with brine, dried over sodium sulfate, filtered and concentrated to dryness to afford 193 (2.2 g, 42%) as white solid. ¹H NMR (400 MHz, DMSO-d₆) δ 8.00 (s, 2H), 4.29 (q, J=7.2 Hz, 2H), 1.27 (t, J=7.2 Hz, 3H); LC-MS: m/z 224.9 [M+H]⁺.

### Step-2: Ethyl 2-(2-chlorobenzamido)-4-(trifluoromethyl)oxazole-5-carboxylate (194)

To a stirred mixture of ethyl 2-amino-4-(trifluoromethyl)oxazole-5-carboxylate (193) (650 mg, 2.90 mmol, 1 eq) in DMF (10 mL), HATU (2.2 g, 5.80 mmol, 2 eq) and DIPEA (1.47 mL, 8.7 mmol, 3.0 eq) were added at RT. The mixture was stirred at same temperature for 30 min. Then 2-chlorobenzoic acid (33) (543 mg, 3.48 mmol, 1.2 eq) was added into the reaction mixture and allowed to stirred at RT for 12 h. The progress of the reaction was monitored by TLC (M.Ph: 5% MeOH in DCM). The reaction mixture diluted with water (75 mL) and extracted with EtOAc (2×150 mL). The combined organic extract was washed with brine, dried over sodium sulfate, filtered and concentrated to dryness. The crude was purified by silica gel column chromatography (elution: 10-40% EtOAc in n-hexane) to afford 194 (458 mg, 44%) as an off-white solid. ¹H NMR (400 MHz, DMSO-d₆) δ 12.77 (s, 1H), 8.00 (s, 1H), 7.65 (d, J=8 Hz, 1H), 7.59-7.53 (m, 2H), 7.49-7.43 (m, 1H), 4.39 (q, J=7.2 Hz, 2H), 1.32 (t, J=7.2 Hz, 3H); LC-MS: m/z 362.95 [M+H]⁺.

### Step-3: Ethyl 2-(2-chloro-N-methylbenzamido)-4-(trifluoromethyl)oxazole-5-carboxylate (195)

To a stirred solution of ethyl 2-(2-chlorobenzamido)-4-(trifluoromethyl)oxazole-5-carboxylate (194) (450 mg, 1.24 mmol, 1 eq) in ACN (10 mL), K₂CO₃ (428 mg, 3.10 mmol, 2.5 eq) and Mel (0.24 mL, 3.72 mmol, 3 eq) were added at 0° C. The reaction mixture was stirred at RT for 12 h. The progress of the reaction was monitored by TLC (M.Ph: 20% EtOAc in n-hexane). After completion of reaction, the reaction mixture was filtered through hyflo by giving EtOAc wash. The filtrate was concentrated to dryness. The crude was diluted with water and extracted with diethyl ether (2×150 mL). The organic mass was concentrated to dryness to afford 195 (308 mg, 66%) as a brown viscous oil. ¹H NMR (400 MHz, DMSO-d₆) δ 7.53-7.51 (m, 3H), 7.45-7.40 (m, 1H), 4.25 (q, J=7.2 Hz, 2H), 3.50 (s, 3H), 1.21 (t, J=7.6 Hz, 3H); LC-MS: m/z 377.0 [M+H]⁺.

### Step-4: 2-(2-Chloro-N-methylbenzamido)-4-(trifluoromethyl)oxazole-5-carboxylic acid (196)

To a stirred solution of ethyl 2-(2-chloro-N-methylbenzamido)-4-(trifluoromethyl)oxazole-5-carboxylate (195) (300 mg, 0.79 mmol, 1 eq) in DCM (5 mL), BBr₃ (1 M in DCM, 7.9 mL, 7.9 mmol, 10 eq) was added at 0° C. The reaction mixture was stirred at RT for 12 h. The progress of the reaction was monitored by TLC (M.Ph: 30% EtOAc in n-hexane). After completion of reaction, the reaction mixture was quenched with MeOH (1 mL) followed by ice chilled water. The mixture was extracted with DCM (2×50 mL). The layers were separated and the combined organic extract was concentered to dryness. The residue was triturated with diethyl ether and filtered to afford 196 (158 mg, 57%) as an off-white solid. ¹H NMR (400 MHz, DMSO-d₆) δ 13.44 (s, 1H), 7.78 (d, J=7.6 Hz, 1H), 7.54-7.49 (m, 2H), 7.47-7.44 (m, 2H) 3.48 (s, 3H); LC-MS: m/z 348.9 [M+H]⁺.

### Step-5: Example 78

In line with the General procedure A, 2-(2-chloro-N-methylbenzamido)-4-(trifluoromethyl)oxazole-5-carboxylic acid (196) (150 mg, 0.43 mmol, 1 eq) was coupled with 4-(pyridin-2-yl)thiazol-2-amine (4) (91 mg, 0.51 mmol, 1.2 eq) to afford Example 78 (29 mg, 13%) as an off-white solid. ¹H NMR (400 MHz, DMSO-d₆) δ 13.32 (s, 1H), 8.63 (d, J=4.4 Hz, 1H), 8.03 (d, J=8.4 Hz, 1H), 7.97 (s, 1H), 7.93 (t, J=8.4 Hz, 1H), 7.53-7.50 (m, 3H), 7.49-7.35 (m, 2H), 3.64 (s, 3H); LC-MS: m/z 508.00 [M+H]⁺; HPLC: 98.7%.

The following Examples can be synthesized using General Procedure A, starting with commercially available or known amine and carboxylic acid building blocks. CAS Registry numbers for each amine and carboxylic acid block is indicated in the Table 5.

TABLE 5

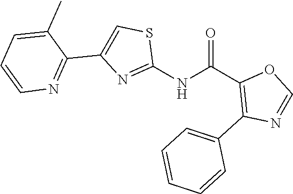 [SMILES: Cc1cccnc1-c1csc(NC(=O)c2ocnc2-c2ccccc2)n1]

| | |
|---|---|
| Acid CAS | 1240599-647 |
| Amino CAS | 1044269-91-1 |

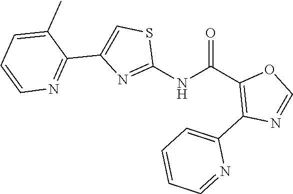 [SMILES: Cc1cccnc1-c1csc(NC(=O)c2ocnc2-c2ccccn2)n1]

| | |
|---|---|
| Acid CAS | 1780365-39-0 |
| Amino CAS | 1044269-91-1 |

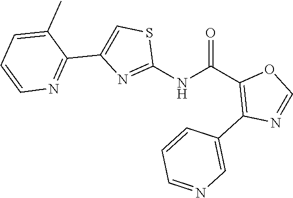 [SMILES: Cc1cccnc1-c1csc(NC(=O)c2ocnc2-c2cccnc2)n1]

| | |
|---|---|
| Acid CAS | 1256811-50-3 |
| Amino CAS | 1044269-91-1 |

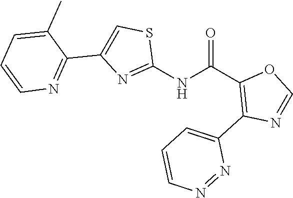 [SMILES: Cc1cccnc1-c1csc(NC(=O)c2ocnc2-c2cccnn2)n1]

| | |
|---|---|
| Acid CAS | 1500565-57-0 |
| Amino CAS | 1044269-91-1 |

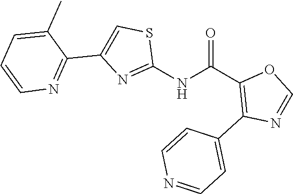 [SMILES: Cc1cccnc1-c1csc(NC(=O)c2ocnc2-c2ccncc2)n1]

| | |
|---|---|
| Acid CAS | 1781745-87-6 |
| Amino CAS | 1044269-91-1 |

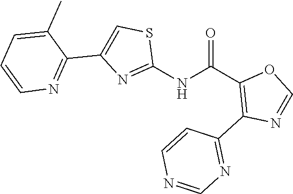 [SMILES: Cc1cccnc1-c1csc(NC(=O)c2ocnc2-c2ccncn2)n1]

| | |
|---|---|
| Acid CAS | 1509416-93-6 |
| Amino CAS | 1044269-91-1 |

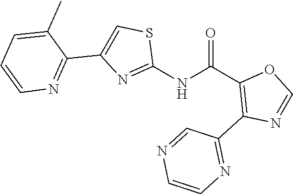 [SMILES: Cc1cccnc1-c1csc(NC(=O)c2ocnc2-c2cnccn2)n1]

| | |
|---|---|
| Acid CAS | 2946657-22-1 |
| Amino CAS | 1044269-91-1 |

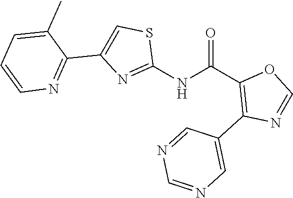 [SMILES: Cc1cccnc1-c1csc(NC(=O)c2ocnc2-c2cncnc2)n1]

| | |
|---|---|
| Acid CAS | 1522470-87-6 |
| Amino CAS | 1044269-91-1 |

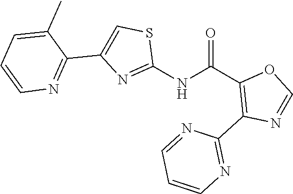 [SMILES: Cc1cccnc1-c1csc(NC(=O)c2ocnc2-c2ncccn2)n1]

| | |
|---|---|
| Acid CAS | 2946657-21-0 |
| Amino CAS | 1044269-91-1 |

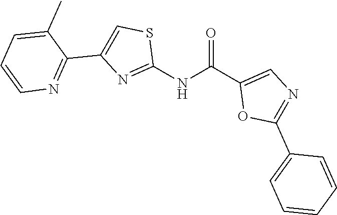 [SMILES: Cc1cccnc1-c1csc(NC(=O)c2cnc(-c3ccccc3)o2)n1]

| | |
|---|---|
| Acid CAS | 199296-83-8 |
| Amino CAS | 1044269-91-1 |

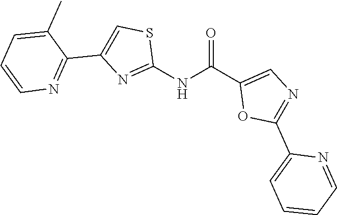 [SMILES: Cc1cccnc1-c1csc(NC(=O)c2cnc(-c3ccccn3)o2)n1]

| | |
|---|---|
| Acid CAS | 1211521-26-4 |
| Amino CAS | 1044269-91-1 |

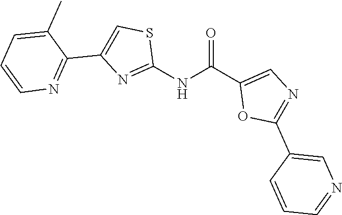 [SMILES: Cc1cccnc1-c1csc(NC(=O)c2cnc(-c3cccnc3)o2)n1]

| | |
|---|---|
| Acid CAS | 1267456-45-0 |
| Amino CAS | 1044269-91-1 |

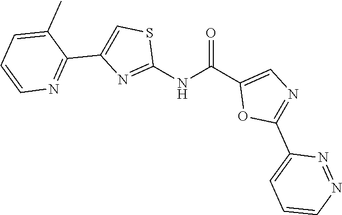 [SMILES: Cc1cccnc1-c1csc(NC(=O)c2cnc(-c3cccnn3)o2)n1]

| | |
|---|---|
| Acid CAS | 2275065-10-4 |
| Amino CAS | 1044269-91-1 |

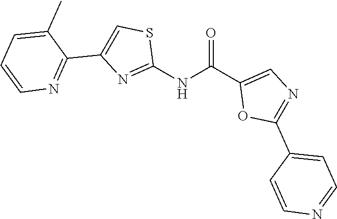 [SMILES: Cc1cccnc1-c1csc(NC(=O)c2cnc(-c3ccncc3)o2)n1]

| | |
|---|---|
| Acid CAS | 1268140-84-6 |
| Amino CAS | 1044269-91-1 |

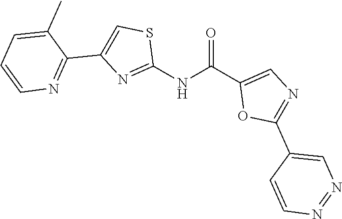 [SMILES: Cc1cccnc1-c1csc(NC(=O)c2cnc(-c3ccnnc3)o2)n1]

| | |
|---|---|
| Acid CAS | 1820884-55-6 |
| Amino CAS | 1044289-91-1 |

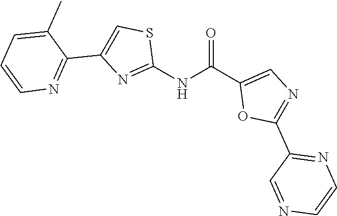 [SMILES: Cc1cccnc1-c1csc(NC(=O)c2cnc(-c3cnccn3)o2)n1]

| | |
|---|---|
| Acid CAS | 2105029-45-4 |
| Amino CAS | 1044269-91-1 |

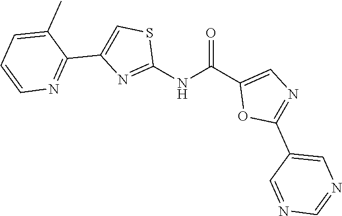 [SMILES: Cc1cccnc1-c1csc(NC(=O)c2cnc(-c3cncnc3)o2)n1]

| | |
|---|---|
| Acid CAS | 1391828-85-5 |
| Amino CAS | 1044269-91-1 |

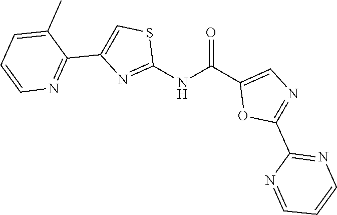 [SMILES: Cc1cccnc1-c1csc(NC(=O)c2cnc(-c3ncccn3)o2)n1]

| | |
|---|---|
| Acid CAS | 2355897-61-7 |
| Amino CAS | 1044269-91-1 |

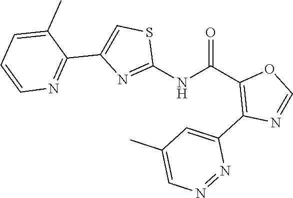 [SMILES: Cc1cnnc(-c2ncoc2C(=O)Nc2nc(-c3ncccc3C)cs2)c1]

| | |
|---|---|
| Acid CAS | 1519469-00-1 |
| Amino CAS | 1044269-91-1 |

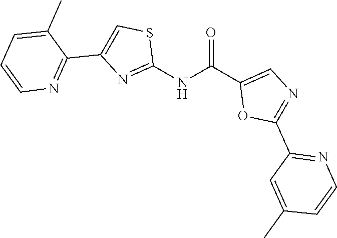 [SMILES: Cc1ccnc(-c2ncc(C(=O)Nc3nc(-c4ncccc4C)cs3)o2)c1]

| | |
|---|---|
| Acid CAS | 2111460-15-0 |
| Amino CAS | 1044269-91-1 |

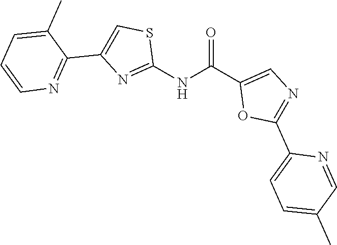 [SMILES: Cc1ccc(-c2ncc(C(=O)Nc3nc(-c4ncccc4C)cs3)o2)nc1]

| | |
|---|---|
| Acid CAS | 2299198-14-2 |
| Amino CAS | 1044269-91-1 |

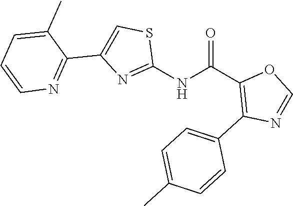 [SMILES: Cc1ccc(-c2ncoc2C(=O)Nc2nc(-c3ncccc3C)cs2)cc1]

| | |
|---|---|
| Acid CAS | 1368803-58-0 |
| Amino CAS | 1044269-91-1 |

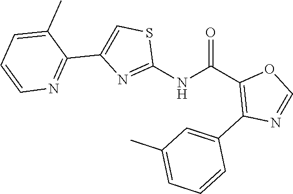 [SMILES: Cc1cccc(-c2ncoc2C(=O)Nc2nc(-c3ncccc3C)cs2)c1]

| | |
|---|---|
| Acid CAS | 1782341-23-4 |
| Amino CAS | 1044269-91-1 |

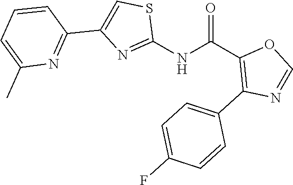 [SMILES: Cc1cccc(-c2csc(NC(=O)c3ocnc3-c3ccc(F)cc3)n2)n1]

| | |
|---|---|
| Acid CAS | 1368699-73-3 |
| Amino CAS | 99358-99-3 |

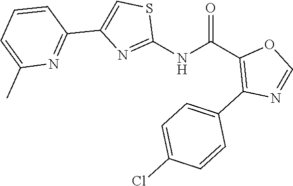 [SMILES: Cc1cccc(-c2csc(NC(=O)c3ocnc3-c3ccc(Cl)cc3)n2)n1]

| | |
|---|---|
| Acid CAS | 1368997-23-2 |
| Amino CAS | 99358-99-3 |

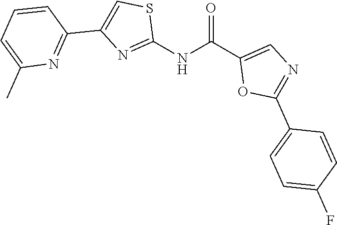 [SMILES: Cc1cccc(-c2csc(NC(=O)c3cnc(-c4ccc(F)cc4)o3)n2)n1]

| | |
|---|---|
| Acid CAS | 1063245-96-0 |
| Amino CAS | 99358-99-3 |

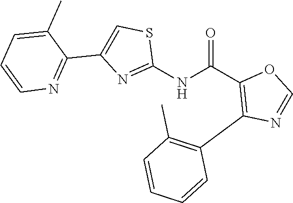 [SMILES: Cc1ccccc1-c1ncoc1C(=O)Nc1nc(-c2ncccc2C)cs1]

| | |
|---|---|
| Acid CAS | 1391739-95-9 |
| Amino CAS | 1044269-91-1 |

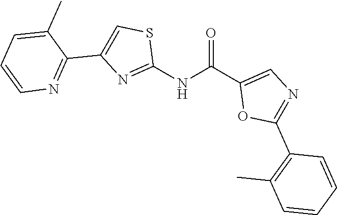 [SMILES: Cc1ccccc1-c1ncc(C(=O)Nc2nc(-c3ncccc3C)cs2)o1]

| | |
|---|---|
| Acid CAS | 1267982-14-8 |
| Amino CAS | 1044269-91-1 |

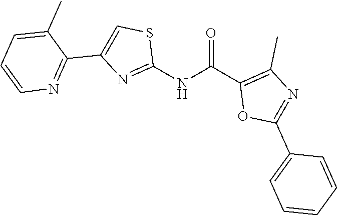 [SMILES: Cc1cccnc1-c1csc(NC(=O)c2oc(-c3ccccc3)nc2C)n1]

| | |
|---|---|
| Acid CAS | 91137-55-2 |
| Amino CAS | 1522975-37-6 |

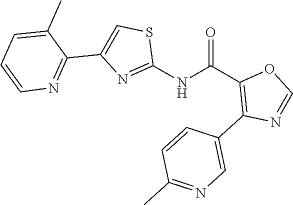 [SMILES: Cc1ccc(-c2ncoc2C(=O)Nc2nc(-c3ncccc3C)cs2)cn1]

| | |
|---|---|
| Acid CAS | 1522975-37-6 |
| Amino CAS | 1044269-91-1 |

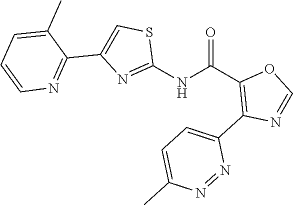 [SMILES: Cc1ccc(-c2ncoc2C(=O)Nc2nc(-c3ncccc3C)cs2)nn1]

| | |
|---|---|
| Acid CAS | 1521338-17-9 |
| Amino CAS | 1044269-91-1 |

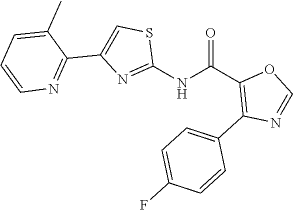 [SMILES: Cc1cccnc1-c1csc(NC(=O)c2ocnc2-c2ccc(F)cc2)n1]

| | |
|---|---|
| Acid CAS | 1368699-73-3 |
| Amino CAS | 1044269-91-1 |

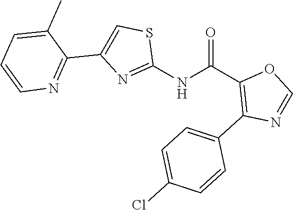 [SMILES: Cc1cccnc1-c1csc(NC(=O)c2ocnc2-c2ccc(Cl)cc2)n1]

| | |
|---|---|
| Acid CAS | 1368997-23-2 |
| Amino CAS | 1044269-91-1 |

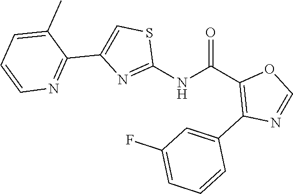 [SMILES: Cc1cccnc1-c1csc(NC(=O)c2ocnc2-c2cccc(F)c2)n1]

| | |
|---|---|
| Acid CAS | 2091101-47-0 |
| Amino CAS | 1044269-91-1 |

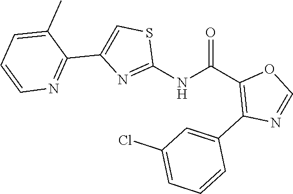 [SMILES: Cc1cccnc1-c1csc(NC(=O)c2ocnc2-c2cccc(Cl)c2)n1]

| | |
|---|---|
| Acid CAS | 2092744-95-9 |
| Amino CAS | 1044269-91-1 |

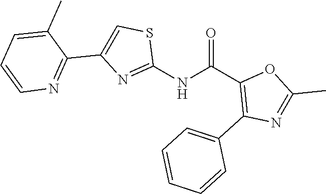 [SMILES: Cc1nc(-c2ccccc2)c(C(=O)Nc2nc(-c3ncccc3C)cs2)o1]

| | |
|---|---|
| Acid CAS | 51143-21-6 |
| Amino CAS | 1044269-91-1 |

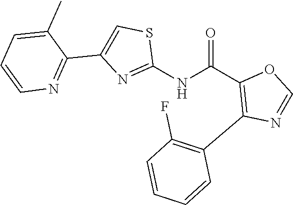 [SMILES: Cc1cccnc1-c1csc(NC(=O)c2ocnc2-c2ccccc2F)n1]

| | |
|---|---|
| Acid CAS | 2092570-24-4 |
| Amino CAS | 1044289-91-1 |

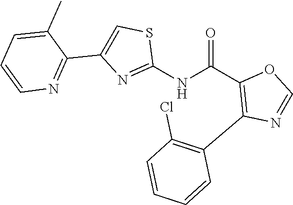 [SMILES: Cc1cccnc1-c1csc(NC(=O)c2ocnc2-c2ccccc2Cl)n1]

| | |
|---|---|
| Acid CAS | 2090608-57-2 |
| Amino CAS | 1044269-91-1 |

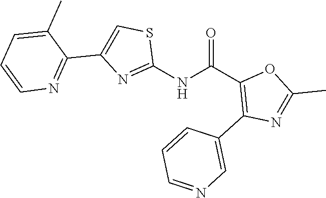 [SMILES: Cc1nc(-c2cccnc2)c(C(=O)Nc2nc(-c3ncccc3C)cs2)o1]

| | |
|---|---|
| Acid CAS | 1368893-76-8 |
| Amino CAS | 1044269-91-1 |

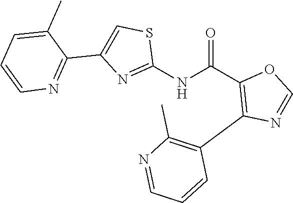 [SMILES: Cc1cccnc1-c1csc(NC(=O)c2ocnc2-c2cccnc2C)n1]

| | |
|---|---|
| Acid CAS | 1526012-57-6 |
| Amino CAS | 1044269-91-1 |

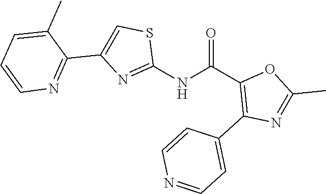 [SMILES: Cc1nc(-c2ccncc2)c(C(=O)Nc2nc(-c3ncccc3C)cs2)o1]

| | |
|---|---|
| Acid CAS | 1368967-55-8 |
| Amino CAS | 1044269-91-1 |

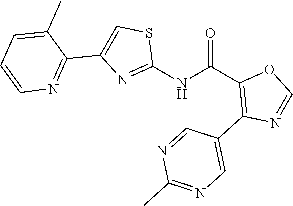 [SMILES: Cc1ncc(-c2ncoc2C(=O)Nc2nc(-c3ncccc3C)cs2)cn1]

| | |
|---|---|
| Acid CAS | 1503017-40-0 |
| Amino CAS | 1044269-91-1 |

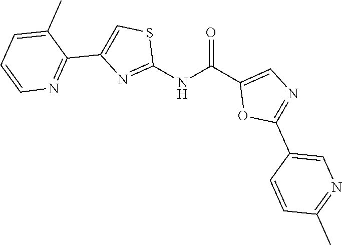 [SMILES: Cc1ccc(-c2ncc(C(=O)Nc3nc(-c4ncccc4C)cs3)o2)cn1]

| | |
|---|---|
| Acid CAS | 2111043-94-6 |
| Amino CAS | 1044269-91-1 |

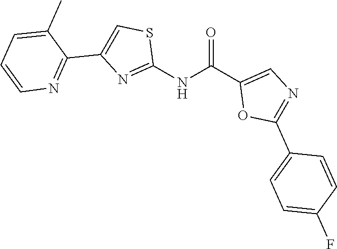 [SMILES: Cc1cccnc1-c1csc(NC(=O)c2cnc(-c3ccc(F)cc3)o2)n1]

| | |
|---|---|
| Acid CAS | 1083245-98-0 |
| Amino CAS | 1044269-91-1 |

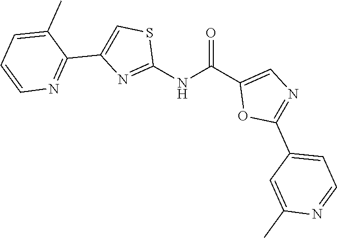 [SMILES: Cc1cc(-c2ncc(C(=O)Nc3nc(-c4ncccc4C)cs3)o2)ccn1]

| | |
|---|---|
| Acid CAS | 1844896-06-5 |
| Amino CAS | 1044269-91-1 |

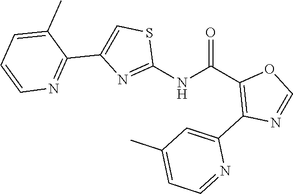 [SMILES: Cc1ccnc(-c2ncoc2C(=O)Nc2nc(-c3ncccc3C)cs2)c1]

| | |
|---|---|
| Acid CAS | 1518655-16-7 |
| Amino CAS | 1044269-91-1 |

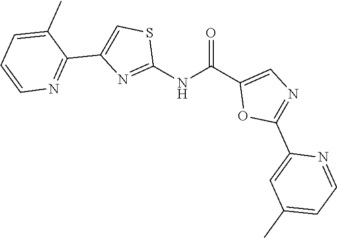 [SMILES: Cc1ccnc(-c2ncc(C(=O)Nc3nc(-c4ncccc4C)cs3)o2)c1]

| | |
|---|---|
| Acid CAS | 2354857-83-1 |
| Amino CAS | 1044269-91-1 |

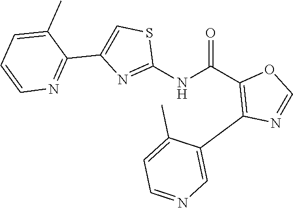 [SMILES: Cc1ccncc1-c1ncoc1C(=O)Nc1nc(-c2ncccc2C)cs1]

| | |
|---|---|
| Acid CAS | 1500316-09-5 |
| Amino CAS | 1044269-91-1 |

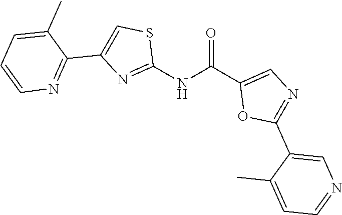 [SMILES: Cc1ccncc1-c1ncc(C(=O)Nc2nc(-c3ncccc3C)cs2)o1]

| | |
|---|---|
| Acid CAS | 2109728-01-8 |
| Amino CAS | 1044269-91-1 |

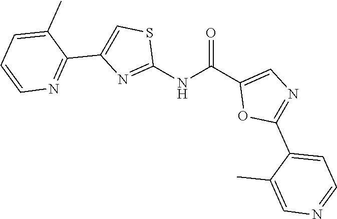 [SMILES: Cc1cnccc1-c1ncc(C(=O)Nc2nc(-c3ncccc3C)cs2)o1]

| | |
|---|---|
| Acid CAS | 2105585-61-1 |
| Amino CAS | 1044269-91-1 |

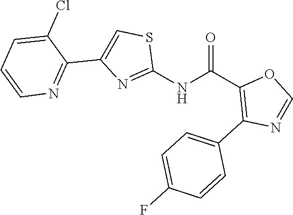 [SMILES: O=C(Nc1nc(-c2ncccc2Cl)cs1)c1ocnc1-c1ccc(F)cc1]

| | |
|---|---|
| Acid CAS | 1368699-73-3 |
| Amino CAS | 2150665-06-0 |

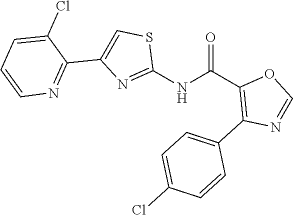 [SMILES: O=C(Nc1nc(-c2ncccc2Cl)cs1)c1ocnc1-c1ccc(Cl)cc1]

| | |
|---|---|
| Acid CAS | 1368997-23-2 |
| Amino CAS | 2150665-06-6 |

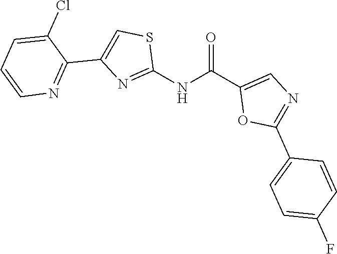 [SMILES: O=C(Nc1nc(-c2ncccc2Cl)cs1)c1cnc(-c2ccc(F)cc2)o1]

| | |
|---|---|
| Acid CAS | 1083245-98-0 |
| Amino CAS | 2150665-06-6 |

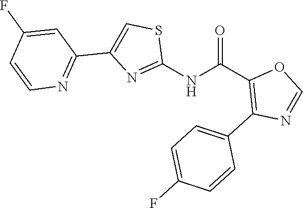 [SMILES: O=C(Nc1nc(-c2cc(F)ccn2)cs1)c1ocnc1-c1ccc(F)cc1]

| | |
|---|---|
| Acid CAS | 1368699-73-3 |
| Amino CAS | 2226222-84-0 |

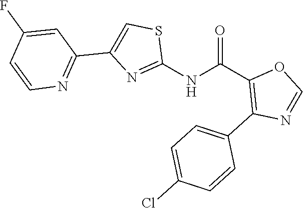 [SMILES: O=C(Nc1nc(-c2cc(F)ccn2)cs1)c1ocnc1-c1ccc(Cl)cc1]

| | |
|---|---|
| Acid CAS | 1368997-23-2 |
| Amino CAS | 2228222-84-0 |

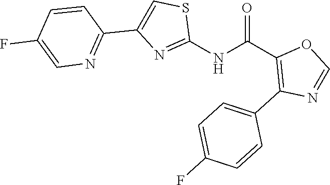 [SMILES: O=C(Nc1nc(-c2ccc(F)cn2)cs1)c1ocnc1-c1ccc(F)cc1]

| | |
|---|---|
| Acid CAS | 1368699-73-3 |
| Amino CAS | 1551651-30-9 |

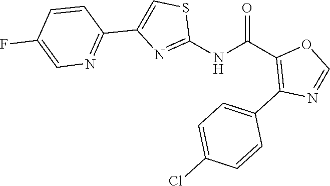 [SMILES: O=C(Nc1nc(-c2ccc(F)cn2)cs1)c1ocnc1-c1ccc(Cl)cc1]

| | |
|---|---|
| Acid CAS | 1368997-23-2 |
| Amino CAS | 1551651-30-9 |

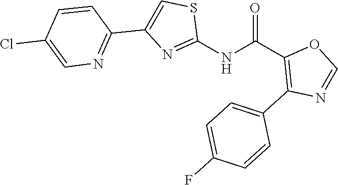 [SMILES: O=C(Nc1nc(-c2ccc(Cl)cn2)cs1)c1ocnc1-c1ccc(F)cc1]

| | |
|---|---|
| Acid CAS | 1368699-73-3 |
| Amino CAS | 1185315-17-6 |

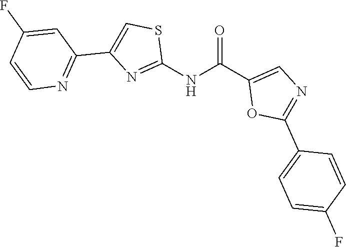 [SMILES: O=C(Nc1nc(-c2cc(F)ccn2)cs1)c1cnc(-c2ccc(F)cc2)o1]

| | |
|---|---|
| Acid CAS | 1083245-98-0 |
| Amino CAS | 2228222-84-0 |

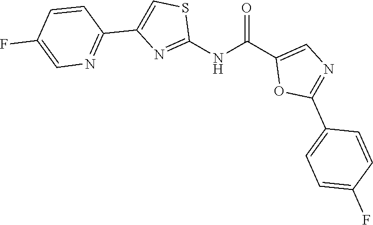 [SMILES: O=C(Nc1nc(-c2ccc(F)cn2)cs1)c1cnc(-c2ccc(F)cc2)o1]

| | |
|---|---|
| Acid CAS | 1083245-98-0 |
| Amino CAS | 1551651-30-9 |

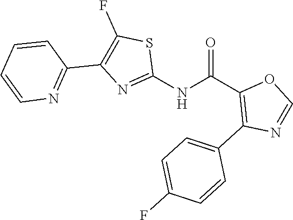 [SMILES: O=C(Nc1nc(-c2ccccn2)c(F)s1)c1ocnc1-c1ccc(F)cc1]

| | |
|---|---|
| Acid CAS | 1368699-73-3 |
| Amino CAS | 1044270-93-0 |

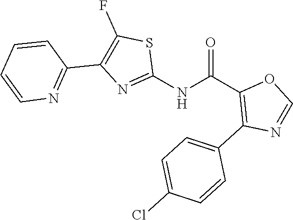 [SMILES: O=C(Nc1nc(-c2ccccn2)c(F)s1)c1ocnc1-c1ccc(Cl)cc1]

| | |
|---|---|
| Acid CAS | 1368997-23-2 |
| Amino CAS | 1044270-93-0 |

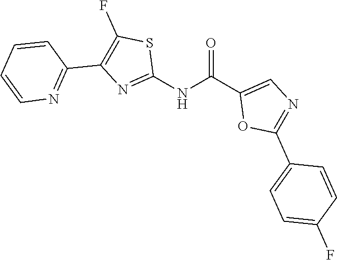 [SMILES: O=C(Nc1nc(-c2ccccn2)c(F)s1)c1cnc(-c2ccc(F)cc2)o1]

| | |
|---|---|
| Acid CAS | 1083245-98-0 |
| Amino CAS | 1044270-93-0 |

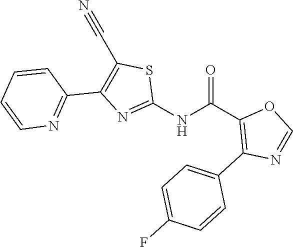 [SMILES: N#Cc1sc(NC(=O)c2ocnc2-c2ccc(F)cc2)nc1-c1ccccn1]

| | |
|---|---|
| Acid CAS | 1368699-73-3 |
| Amino CAS | 1139743-06-8 |

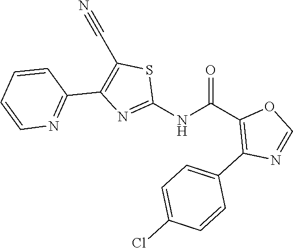 [SMILES: N#Cc1sc(NC(=O)c2ocnc2-c2ccc(Cl)cc2)nc1-c1ccccn1]

| | |
|---|---|
| Acid CAS | 1368997-23-2 |
| Amino CAS | 1139743-06-8 |

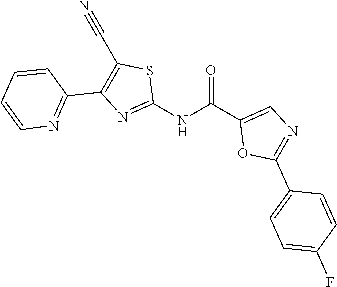 [SMILES: N#Cc1sc(NC(=O)c2cnc(-c3ccc(F)cc3)o2)nc1-c1ccccn1]

| | |
|---|---|
| Acid CAS | 1083245-98-0 |
| Amino CAS | 1139743-06-8 |

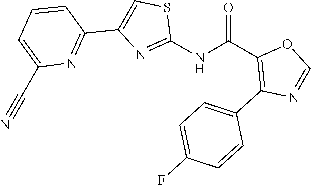 [SMILES: N#Cc1cccc(-c2csc(NC(=O)c3ocnc3-c3ccc(F)cc3)n2)n1]

| | |
|---|---|
| Acid CAS | 1388699-73-3 |
| Amino CAS | 1185317-90-1 |

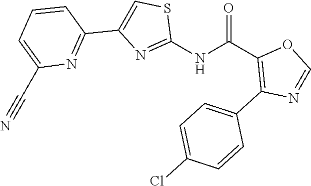 [SMILES: N#Cc1cccc(-c2csc(NC(=O)c3ocnc3-c3ccc(Cl)cc3)n2)n1]

| | |
|---|---|
| Acid CAS | 1368997-23-2 |
| Amino CAS | 1185317-90-1 |

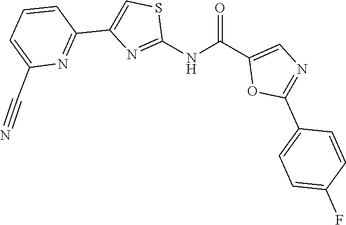 [SMILES: N#Cc1cccc(-c2csc(NC(=O)c3cnc(-c4ccc(F)cc4)o3)n2)n1]

| | |
|---|---|
| Acid CAS | 1083245-98-0 |
| Amino CAS | 1185317-90-1 |

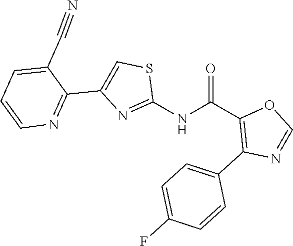 [SMILES: N#Cc1cccnc1-c1csc(NC(=O)c2ocnc2-c2ccc(F)cc2)n1]

| | |
|---|---|
| Acid CAS | 1368699-73-3 |
| Amino CAS | 2253868-93-6 |

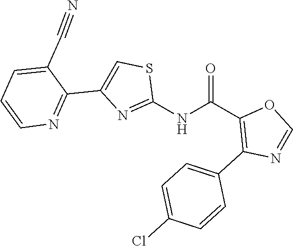 [SMILES: N#Cc1cccnc1-c1csc(NC(=O)c2ocnc2-c2ccc(Cl)cc2)n1]

| | |
|---|---|
| Acid CAS | 1368997-23-2 |
| Amino CAS | 2253868-93-6 |

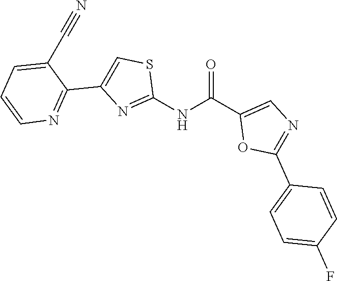 [SMILES: N#Cc1cccnc1-c1csc(NC(=O)c2cnc(-c3ccc(F)cc3)o2)n1]

| | |
|---|---|
| Acid CAS | 1083245-98-0 |
| Amino CAS | 2253868-93-6 |

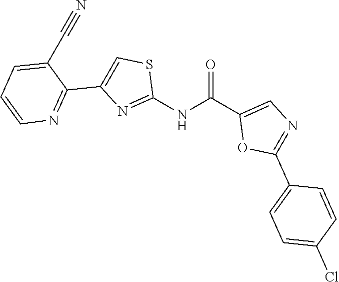 [SMILES: N#Cc1cccnc1-c1csc(NC(=O)c2cnc(-c3ccc(Cl)cc3)o2)n1]

| | |
|---|---|
| Acid CAS | 1082076-32-1 |
| Amino CAS | 2253368-93-6 |

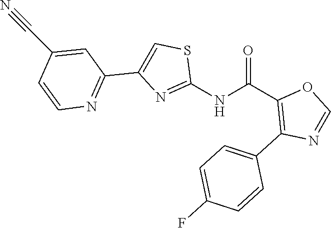 [SMILES: N#Cc1ccnc(-c2csc(NC(=O)c3ocnc3-c3ccc(F)cc3)n2)c1]

| | |
|---|---|
| Acid CAS | 1368699-73-3 |
| Amino CAS | 2253868-97-0 |

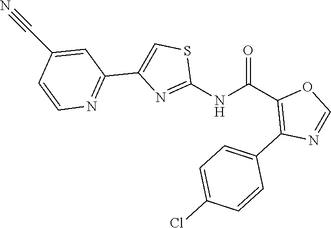 [SMILES: N#Cc1ccnc(-c2csc(NC(=O)c3ocnc3-c3ccc(Cl)cc3)n2)c1]

| | |
|---|---|
| Acid CAS | 1368997-23-2 |
| Amino CAS | 2253868-97-0 |

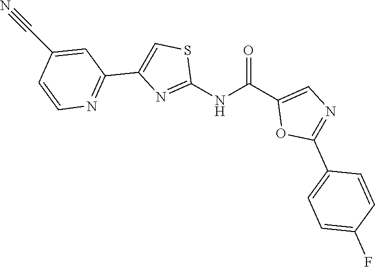 [SMILES: N#Cc1ccnc(-c2csc(NC(=O)c3cnc(-c4ccc(F)cc4)o3)n2)c1]

| | |
|---|---|
| Acid CAS | 1083245-98-0 |
| Amino CAS | 2253868-97-0 |

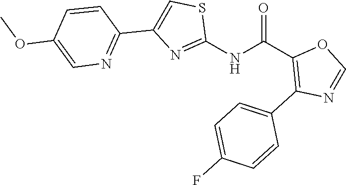 [SMILES: COc1ccc(-c2csc(NC(=O)c3ocnc3-c3ccc(F)cc3)n2)nc1]

| | |
|---|---|
| Acid CAS | 1368699-73-3 |
| Amino CAS | 2228346-85-6 |

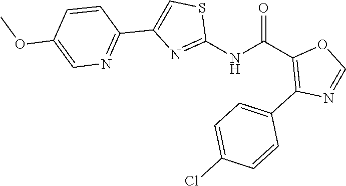 [SMILES: COc1ccc(-c2csc(NC(=O)c3ocnc3-c3ccc(Cl)cc3)n2)nc1]

| | |
|---|---|
| Acid CAS | 1368997-23-2 |
| Amino CAS | 2228346-85-6 |

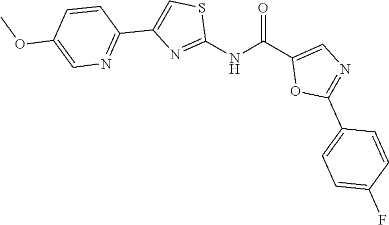 [SMILES: COc1ccc(-c2csc(NC(=O)c3cnc(-c4ccc(F)cc4)o3)n2)nc1]

| | |
|---|---|
| Acid CAS | 1083245-98-0 |
| Amino CAS | 2228348-85-0 |

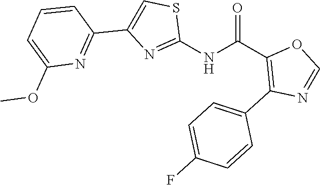 [SMILES: COc1cccc(-c2csc(NC(=O)c3ocnc3-c3ccc(F)cc3)n2)n1]

| | |
|---|---|
| Acid CAS | 1366699-73-3 |
| Amino CAS | 1870028-77-5 |

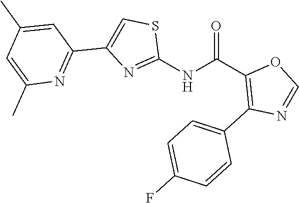 [SMILES: Cc1cc(C)nc(-c2csc(NC(=O)c3ocnc3-c3ccc(F)cc3)n2)c1]

| | |
|---|---|
| Acid CAS | 1368099-73-3 |
| Amino CAS | 1865056-88-7 |

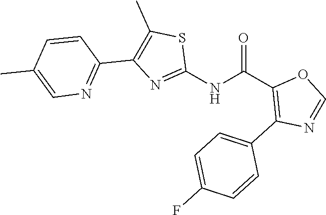 [SMILES: Cc1ccc(-c2nc(NC(=O)c3ocnc3-c3ccc(F)cc3)sc2C)nc1]

| | |
|---|---|
| Acid CAS | 1368699-73-3 |
| Amino CAS | 2294596-48-6 |

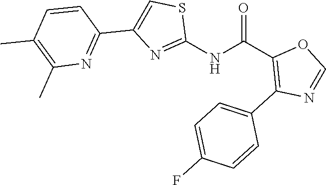 [SMILES: Cc1ccc(-c2csc(NC(=O)c3ocnc3-c3ccc(F)cc3)n2)nc1C]

| | |
|---|---|
| Acid CAS | 1368699-73-3 |
| Amino CAS | 1934459-93-4 |

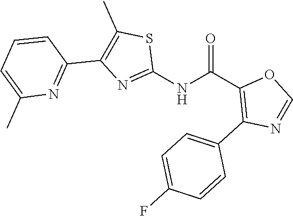 [SMILES: Cc1cccc(-c2nc(NC(=O)c3ocnc3-c3ccc(F)cc3)sc2C)n1]

| | |
|---|---|
| Acid CAS | 1368699-73-3 |
| Amino CAS | 2169472-77-7 |

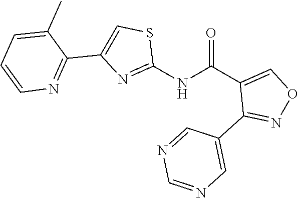 [SMILES: Cc1cccnc1-c1csc(NC(=O)c2conc2-c2cncnc2)n1]

| | |
|---|---|
| Acid CAS | 2023266-77-3 |
| Amino CAS | 1044269-91-1 |

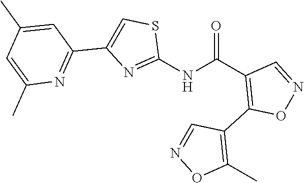 [SMILES: Cc1cc(C)nc(-c2csc(NC(=O)c3cnoc3-c3cnoc3C)n2)c1]

| | |
|---|---|
| Acid CAS | 2091354-55-9 |
| Amino CAS | 1665056-86-7 |

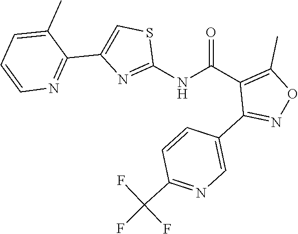 [SMILES: Cc1cccnc1-c1csc(NC(=O)c2c(-c3ccc(C(F)(F)F)nc3)noc2C)n1]

| | |
|---|---|
| Acid CAS | 2230011-77-3 |
| Amino CAS | 1044269-91-1 |

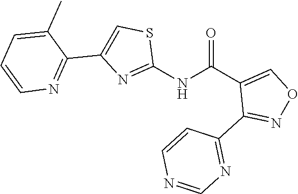 [SMILES: Cc1cccnc1-c1csc(NC(=O)c2conc2-c2ccncn2)n1]

| | |
|---|---|
| Acid CAS | 1083369-25-8 |
| Amino CAS | 1044269-91-1 |

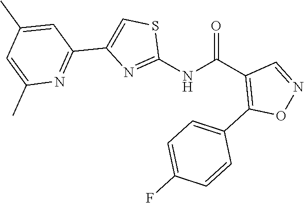 [SMILES: Cc1cc(C)nc(-c2csc(NC(=O)c3cnoc3-c3ccc(F)cc3)n2)c1]

| | |
|---|---|
| Acid CAS | 618383-51-0 |
| Amino CAS | 1865056-88-7 |

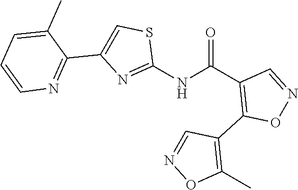 [SMILES: Cc1cccnc1-c1csc(NC(=O)c2cnoc2-c2cnoc2C)n1]

| | |
|---|---|
| Acid CAS | 2091354-55-9 |
| Amino CAS | 1044269-91-1 |

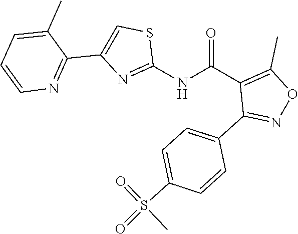 [SMILES: Cc1cccnc1-c1csc(NC(=O)c2c(-c3ccc(S(C)(=O)=O)cc3)noc2C)n1]

| | |
|---|---|
| Acid CAS | 1187543-80-1 |
| Amino CAS | 1044269-91-1 |

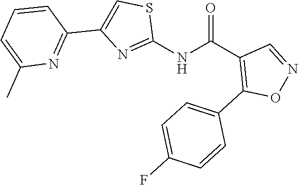 [SMILES: Cc1cccc(-c2csc(NC(=O)c3cnoc3-c3ccc(F)cc3)n2)n1]

| | |
|---|---|
| Acid CAS | 618383-51-0 |
| Amino CAS | 99358-99-3 |

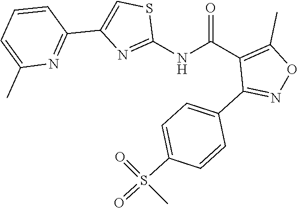 [SMILES: Cc1cccc(-c2csc(NC(=O)c3c(-c4ccc(S(C)(=O)=O)cc4)noc3C)n2)n1]

| | |
|---|---|
| Acid CAS | 1187543-80-1 |
| Amino CAS | 99358-99-3 |

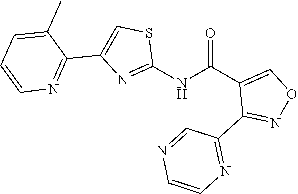 [SMILES: Cc1cccnc1-c1csc(NC(=O)c2conc2-c2cnccn2)n1]

| | |
|---|---|
| Acid CAS | 1083246-23-4 |
| Amino CAS | 1044269-91-1 |

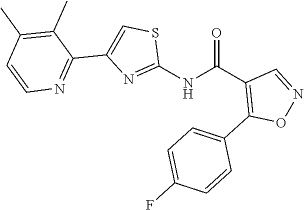 [SMILES: Cc1ccnc(-c2csc(NC(=O)c3cnoc3-c3ccc(F)cc3)n2)c1C]

| | |
|---|---|
| Acid CAS | 618363-51-0 |
| Amino CAS | 2169166-95-2 |

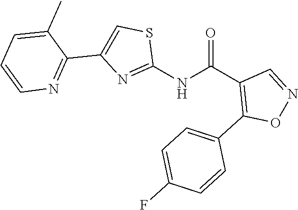 [SMILES: Cc1cccnc1-c1csc(NC(=O)c2cnoc2-c2ccc(F)cc2)n1]

| | |
|---|---|
| Acid CAS | 618383-51-0 |
| Amino CAS | 1044269-91-1 |

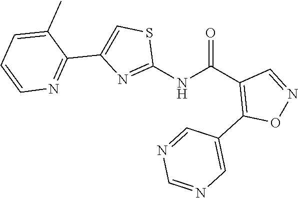 [SMILES: Cc1cccnc1-c1csc(NC(=O)c2cnoc2-c2cncnc2)n1]

| | |
|---|---|
| Acid CAS | 1498038-23-5 |
| Amino CAS | 1044269-91-1 |

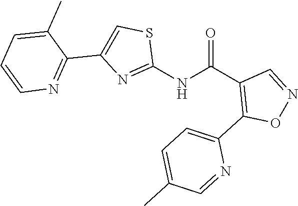 [SMILES: Cc1ccc(-c2oncc2C(=O)Nc2nc(-c3ncccc3C)cs2)nc1]

| | |
|---|---|
| Acid CAS | 2229072-01-7 |
| Amino CAS | 1044269-91-1 |

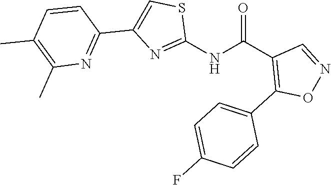 [SMILES: Cc1ccc(-c2csc(NC(=O)c3cnoc3-c3ccc(F)cc3)n2)nc1C]

| | |
|---|---|
| Acid CAS | 618363-51-0 |
| Amino CAS | 1934459-93-4 |

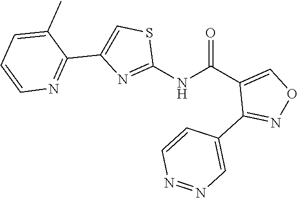 [SMILES: Cc1cccnc1-c1csc(NC(=O)c2conc2-c2ccnnc2)n1]

| | |
|---|---|
| Acid CAS | 1083246-21-2 |
| Amino CAS | 1044269-91-1 |

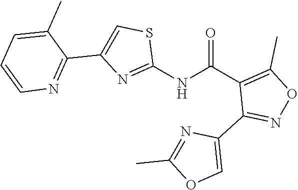 [SMILES: Cc1nc(-c2noc(C)c2C(=O)Nc2nc(-c3ncccc3C)cs2)co1]

| | |
|---|---|
| Acid CAS | 2215069-38-6 |
| Amino CAS | 1044269-91-1 |

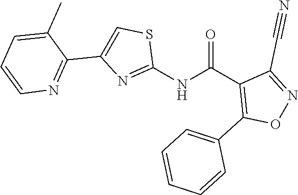 [SMILES: Cc1cccnc1-c1csc(NC(=O)c2c(C#N)noc2-c2ccccc2)n1]

| | |
|---|---|
| Acid CAS | 2092849-57-3 |
| Amino CAS | 1044269-91-1 |

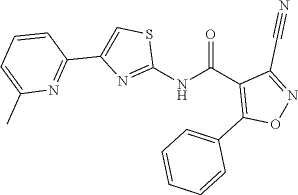 [SMILES: Cc1cccc(-c2csc(NC(=O)c3c(C#N)noc3-c3ccccc3)n2)n1]

| | |
|---|---|
| Acid CAS | 2092649-57-3 |
| Amino CAS | 99358-99-3 |

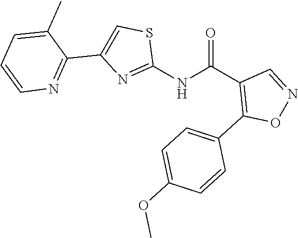 [SMILES: COc1ccc(-c2oncc2C(=O)Nc2nc(-c3ncccc3C)cs2)cc1]

| | |
|---|---|
| Acid CAS | 618383-50-9 |
| Amino CAS | 1044269-91-1 |

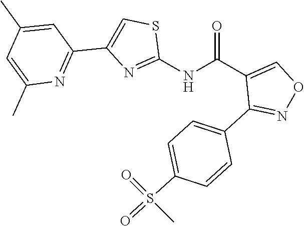 [SMILES: Cc1cc(C)nc(-c2csc(NC(=O)c3conc3-c3ccc(S(C)(=O)=O)cc3)n2)c1]

| | |
|---|---|
| Acid CAS | 681454-62-6 |
| Amino CAS | 1865056-88-7 |

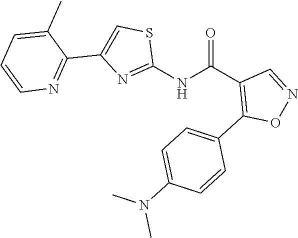 [SMILES: Cc1cccnc1-c1csc(NC(=O)c2cnoc2-c2ccc(N(C)C)cc2)n1]

| | |
|---|---|
| Acid CAS | 2106631-68-7 |
| Amino CAS | 1044269-91-1 |

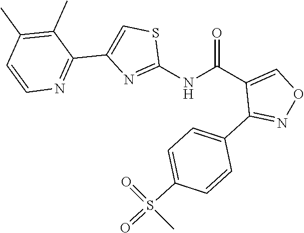 [SMILES: Cc1ccnc(-c2csc(NC(=O)c3conc3-c3ccc(S(C)(=O)=O)cc3)n2)c1C]

| | |
|---|---|
| Acid CAS | 681454-62-6 |
| Amino CAS | 2169186-95-2 |

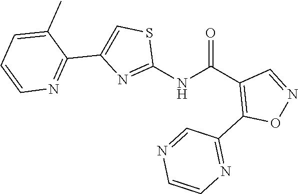 [SMILES: Cc1cccnc1-c1csc(NC(=O)c2cnoc2-c2cnccn2)n1]

| | |
|---|---|
| Acid CAS | 1498959-01-5 |
| Amino CAS | 1044269-91-1 |

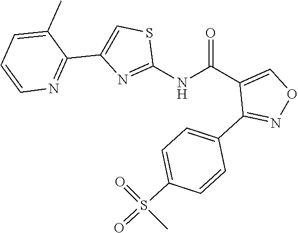 [SMILES: Cc1cccnc1-c1csc(NC(=O)c2conc2-c2ccc(S(C)(=O)=O)cc2)n1]

| | |
|---|---|
| Acid CAS | 681454-62-6 |
| Amino CAS | 1044269-91-1 |

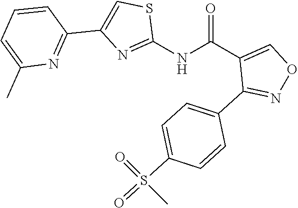 [SMILES: Cc1cccc(-c2csc(NC(=O)c3conc3-c3ccc(S(C)(=O)=O)cc3)n2)n1]

| | |
|---|---|
| Acid CAS | 681454-62-6 |
| Amino CAS | 99358-99-3 |

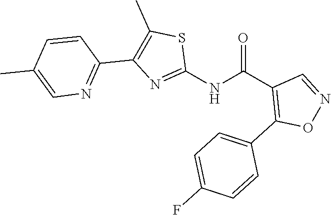 [SMILES: Cc1ccc(-c2nc(NC(=O)c3cnoc3-c3ccc(F)cc3)sc2C)nc1]

| | |
|---|---|
| Acid CAS | 618383-51-0 |
| Amino CAS | 2294596-48-6 |

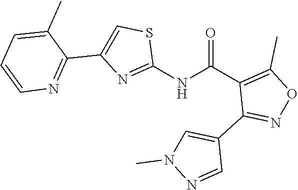 [SMILES: Cc1cccnc1-c1csc(NC(=O)c2c(-c3cnn(C)c3)noc2C)n1]

| | |
|---|---|
| Acid CAS | 1408897-81-1 |
| Amino CAS | 1044269-91-1 |

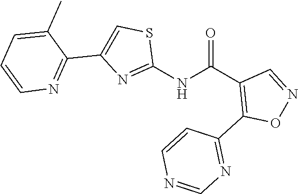 [SMILES: Cc1cccnc1-c1csc(NC(=O)c2cnoc2-c2ccncn2)n1]

| | |
|---|---|
| Acid CAS | 1498675-70-7 |
| Amino CAS | 1044269-91-1 |

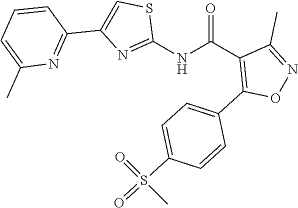 [SMILES: Cc1cccc(-c2csc(NC(=O)c3c(C)noc3-c3ccc(S(C)(=O)=O)cc3)n2)n1]

| | |
|---|---|
| Acid CAS | 91570-24-0 |
| Amino CAS | 99358-99-3 |

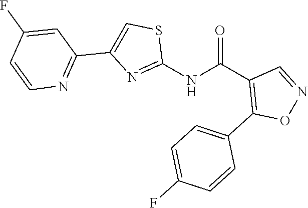 [SMILES: O=C(Nc1nc(-c2cc(F)ccn2)cs1)c1cnoc1-c1ccc(F)cc1]

| | |
|---|---|
| Acid CAS | 618383-51-0 |
| Amino CAS | 2228222-84-0 |

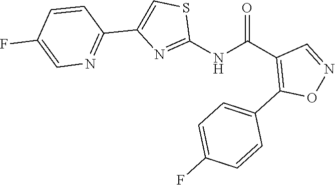 [SMILES: O=C(Nc1nc(-c2ccc(F)cn2)cs1)c1cnoc1-c1ccc(F)cc1]

| | |
|---|---|
| Acid CAS | 618383-51-0 |
| Amino CAS | 1551651-30-9 |

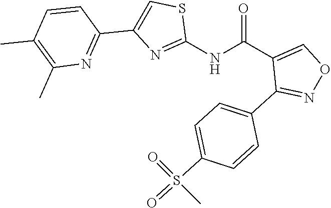 [SMILES: Cc1ccc(-c2csc(NC(=O)c3conc3-c3ccc(S(C)(=O)=O)cc3)n2)nc1C]

| | |
|---|---|
| Acid CAS | 681454-62-6 |
| Amino CAS | 1934459-93-4 |

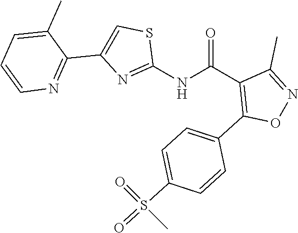 [SMILES: Cc1cccnc1-c1csc(NC(=O)c2c(C)noc2-c2ccc(S(C)(=O)=O)cc2)n1]

| | |
|---|---|
| Acid CAS | 91570-24-0 |
| Amino CAS | 1044289-91-1 |

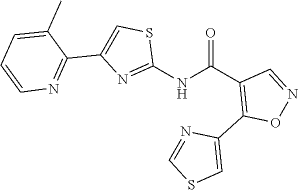 [SMILES: Cc1cccnc1-c1csc(NC(=O)c2cnoc2-c2cscn2)n1]

| | |
|---|---|
| Acid CAS | 2090238-04-1 |
| Amino CAS | 1044269-91-1 |

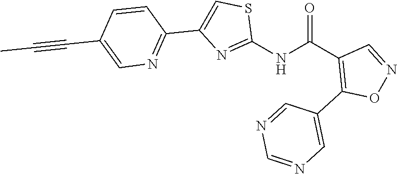 [SMILES: CC#Cc1ccc(-c2csc(NC(=O)c3cnoc3-c3cncnc3)n2)nc1]

| | |
|---|---|
| Acid CAS | 1498038-23-5 |
| Amino CAS | 2592824-00-3 |

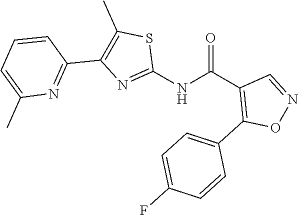 [SMILES: Cc1cccc(-c2nc(NC(=O)c3cnoc3-c3ccc(F)cc3)sc2C)n1]

| | |
|---|---|
| Acid CAS | 618383-51-0 |
| Amino CAS | 2189472-77-7 |

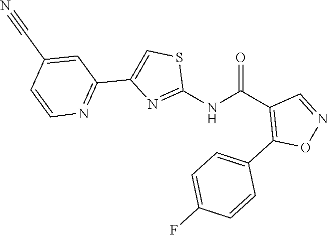 [SMILES: N#Cc1ccnc(-c2csc(NC(=O)c3cnoc3-c3ccc(F)cc3)n2)c1]

| | |
|---|---|
| Acid CAS | 618383-51-0 |
| Amino CAS | 2253868-97-0 |

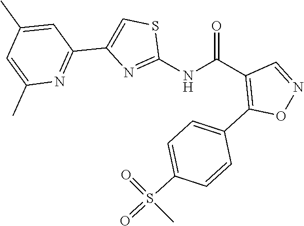 [SMILES: Cc1cc(C)nc(-c2csc(NC(=O)c3cnoc3-c3ccc(S(C)(=O)=O)cc3)n2)c1]

| | |
|---|---|
| Acid CAS | 1368404-20-9 |
| Amino CAS | 1865056-88-7 |

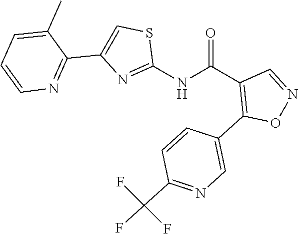 [SMILES: Cc1cccnc1-c1csc(NC(=O)c2cnoc2-c2ccc(C(F)(F)F)nc2)n1]

| | |
|---|---|
| Acid CAS | 2228927-02-2 |
| Amino CAS | 1044269-91-1 |

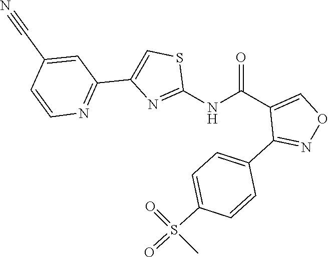 [SMILES: CS(=O)(=O)c1ccc(-c2nocc2C(=O)Nc2nc(-c3cc(C#N)ccn3)cs2)cc1]

| | |
|---|---|
| Acid CAS | 681454-02-6 |
| Amino CAS | 2253868-97-0 |

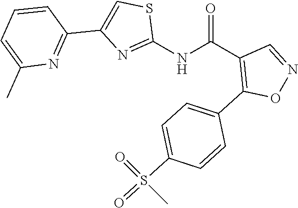 [SMILES: Cc1cccc(-c2csc(NC(=O)c3cnoc3-c3ccc(S(C)(=O)=O)cc3)n2)n1]

| | |
|---|---|
| Acid CAS | 1368404-20-9 |
| Amino CAS | 99358-99-3 |

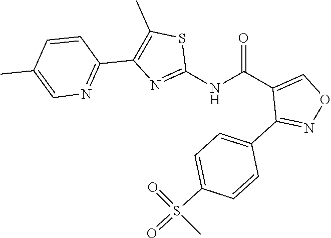 [SMILES: Cc1ccc(-c2nc(NC(=O)c3conc3-c3ccc(S(C)(=O)=O)cc3)sc2C)nc1]

| | |
|---|---|
| Acid CAS | 681454-52-6 |
| Amino CAS | 2294596-48-6 |

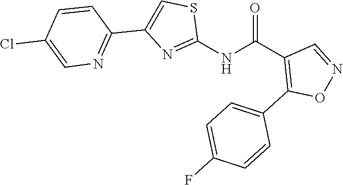 [SMILES: O=C(Nc1nc(-c2ccc(Cl)cn2)cs1)c1cnoc1-c1ccc(F)cc1]

| | |
|---|---|
| Acid CAS | 618383-51-0 |
| Amino CAS | 1185315-17-6 |

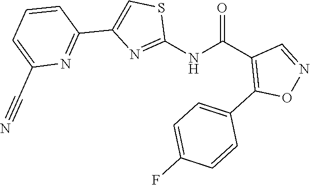 [SMILES: N#Cc1cccc(-c2csc(NC(=O)c3cnoc3-c3ccc(F)cc3)n2)n1]

| | |
|---|---|
| Acid CAS | 618383-51-0 |
| Amino CAS | 1185317-90-1 |

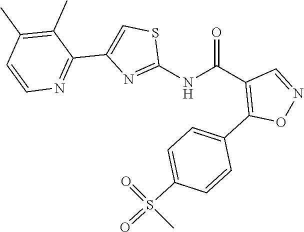 [SMILES: Cc1ccnc(-c2csc(NC(=O)c3cnoc3-c3ccc(S(C)(=O)=O)cc3)n2)c1C]

| | |
|---|---|
| Acid CAS | 1368404-20-9 |
| Amino CAS | 2169166-95-2 |

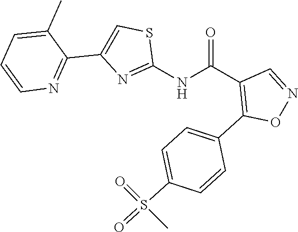 [SMILES: Cc1cccnc1-c1csc(NC(=O)c2cnoc2-c2ccc(S(C)(=O)=O)cc2)n1]

| | |
|---|---|
| Acid CAS | 1368404-20-9 |
| Amino CAS | 1044269-91-1 |

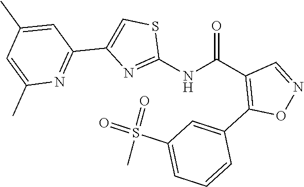 [SMILES: Cc1cc(C)nc(-c2csc(NC(=O)c3cnoc3-c3cccc(S(C)(=O)=O)c3)n2)c1]

| | |
|---|---|
| Acid CAS | 2228628-28-0 |
| Amino CAS | 1865056-88-7 |

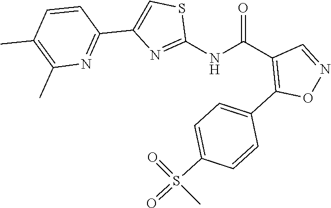 [SMILES: Cc1ccc(-c2csc(NC(=O)c3cnoc3-c3ccc(S(C)(=O)=O)cc3)n2)nc1C]

| | |
|---|---|
| Acid CAS | 1368404-20-9 |
| Amino CAS | 1934459-93-4 |

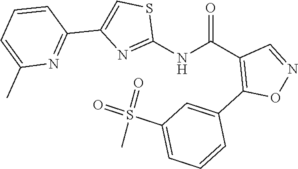 [SMILES: Cc1cccc(-c2csc(NC(=O)c3cnoc3-c3cccc(S(C)(=O)=O)c3)n2)n1]

| | |
|---|---|
| Acid CAS | 2228828-28-0 |
| Amino CAS | 99358-99-3 |

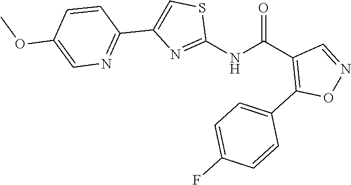 [SMILES: COc1ccc(-c2csc(NC(=O)c3cnoc3-c3ccc(F)cc3)n2)nc1]

| | |
|---|---|
| Acid CAS | 618383-51-0 |
| Amino CAS | 2228346-85-6 |

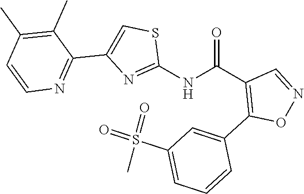 [SMILES: Cc1ccnc(-c2csc(NC(=O)c3cnoc3-c3cccc(S(C)(=O)=O)c3)n2)c1C]

| | |
|---|---|
| Acid CAS | 2228628-28-0 |
| Amino CAS | 2169166-95-2 |

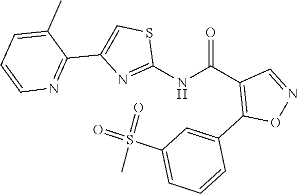 [SMILES: Cc1cccnc1-c1csc(NC(=O)c2cnoc2-c2cccc(S(C)(=O)=O)c2)n1]

| | |
|---|---|
| Acid CAS | 2228628-28-0 |
| Amino CAS | 1044269-91-1 |

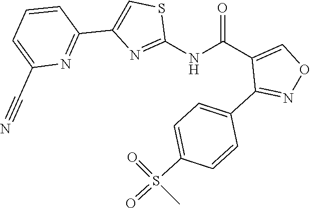 [SMILES: CS(=O)(=O)c1ccc(-c2nocc2C(=O)Nc2nc(-c3cccc(C#N)n3)cs2)cc1]

| | |
|---|---|
| Acid CAS | 681454-62-6 |
| Amino CAS | 1185317-90-1 |

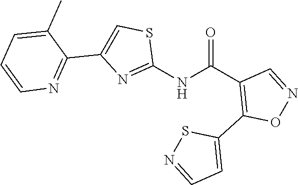 [SMILES: Cc1cccnc1-c1csc(NC(=O)c2cnoc2-c2ccns2)n1]

| | |
|---|---|
| Acid CAS | 2295086-60-9 |
| Amino CAS | 1044269-91-1 |

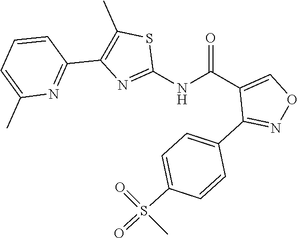 [SMILES: Cc1cccc(-c2nc(NC(=O)c3conc3-c3ccc(S(C)(=O)=O)cc3)sc2C)n1]

| | |
|---|---|
| Acid CAS | 681454-62-6 |
| Amino CAS | 2169472-77-7 |

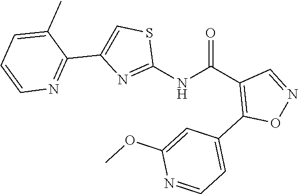 [SMILES: COc1cc(-c2oncc2C(=O)Nc2nc(-c3ncccc3C)cs2)ccn1]

| | |
|---|---|
| Acid CAS | 2228552-56-3 |
| Amino CAS | 1044269-91-1 |

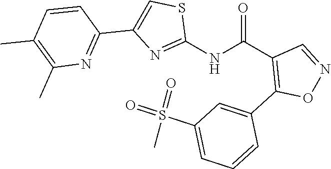 [SMILES: Cc1ccc(-c2csc(NC(=O)c3cnoc3-c3cccc(S(C)(=O)=O)c3)n2)nc1C]

| | |
|---|---|
| Acid CAS | 2228628-28-0 |
| Amino CAS | 1934459-93-4 |

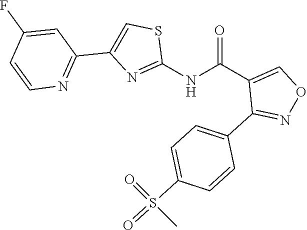 [SMILES: CS(=O)(=O)c1ccc(-c2nocc2C(=O)Nc2nc(-c3cc(F)ccn3)cs2)cc1]

| | |
|---|---|
| Acid CAS | 681454-62-6 |
| Amino CAS | 2228222-84-0 |

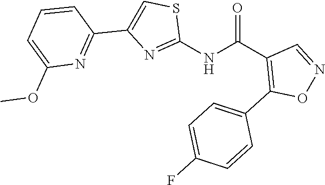 [SMILES: COc1cccc(-c2csc(NC(=O)c3cnoc3-c3ccc(F)cc3)n2)n1]

| | |
|---|---|
| Acid CAS | 618383-51-0 |
| Amino CAS | 1870028-77-5 |

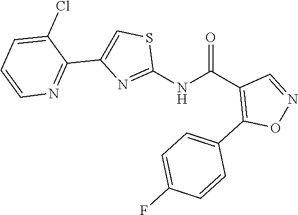 [SMILES: O=C(Nc1nc(-c2ncccc2Cl)cs1)c1cnoc1-c1ccc(F)cc1]

| | |
|---|---|
| Acid CAS | 618383-51-0 |
| Amino CAS | 2150665-06-6 |

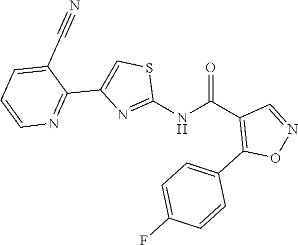 [SMILES: N#Cc1cccnc1-c1csc(NC(=O)c2cnoc2-c2ccc(F)cc2)n1]

| | |
|---|---|
| Acid CAS | 615383-51-0 |
| Amino CAS | 2253868-93-6 |

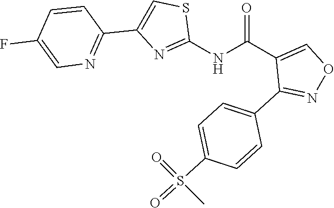 [SMILES: CS(=O)(=O)c1ccc(-c2nocc2C(=O)Nc2nc(-c3ccc(F)cn3)cs2)cc1]

| | |
|---|---|
| Acid CAS | 681454-62-6 |
| Amino CAS | 1551651-30-9 |

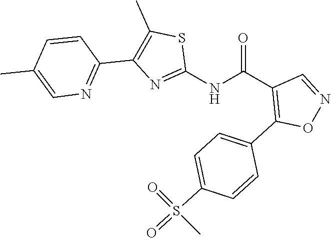 [SMILES: Cc1ccc(-c2nc(NC(=O)c3cnoc3-c3ccc(S(C)(=O)=O)cc3)sc2C)nc1]

| | |
|---|---|
| Acid CAS | 1368404-20-9 |
| Amino CAS | 2294596-48-6 |

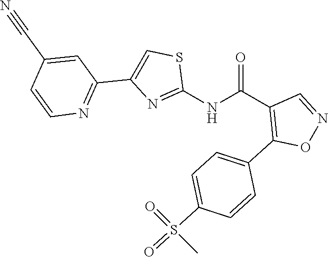 [SMILES: CS(=O)(=O)c1ccc(-c2oncc2C(=O)Nc2nc(-c3cc(C#N)ccn3)cs2)cc1]

| | |
|---|---|
| Acid CAS | 1368404-20-9 |
| Amino CAS | 2253868-97-0 |

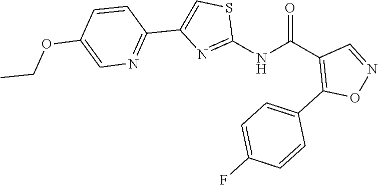 [SMILES: CCOc1ccc(-c2csc(NC(=O)c3cnoc3-c3ccc(F)cc3)n2)nc1]

| | |
|---|---|
| Acid CAS | 618383-51-0 |
| Amino CAS | 2788587-25-1 |

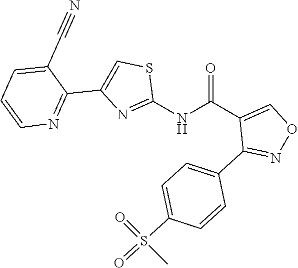 [SMILES: CS(=O)(=O)c1ccc(-c2nocc2C(=O)Nc2nc(-c3ncccc3C#N)cs2)cc1]

| | |
|---|---|
| Acid CAS | 681454-62-6 |
| Amino CAS | 2253868-93-6 |

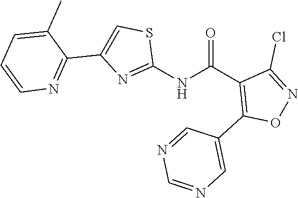 [SMILES: Cc1cccnc1-c1csc(NC(=O)c2c(Cl)noc2-c2cncnc2)n1]

| | |
|---|---|
| Acid CAS | 1481551-61-4 |
| Amino CAS | 1044269-91-1 |

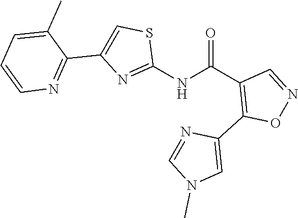 [SMILES: Cc1cccnc1-c1csc(NC(=O)c2cnoc2-c2cn(C)cn2)n1]

| | |
|---|---|
| Acid CAS | 2142487-62-3 |
| Amino CAS | 1044269-91-1 |

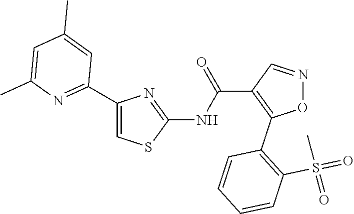 [SMILES: Cc1cc(C)nc(-c2csc(NC(=O)c3cnoc3-c3ccccc3S(C)(=O)=O)n2)c1]

| | |
|---|---|
| Acid CAS | 2228267-03-4 |
| Amino CAS | 1865056-88-7 |

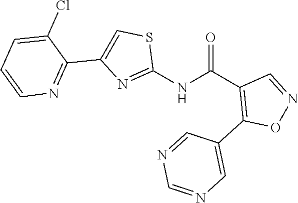 [SMILES: O=C(Nc1nc(-c2ncccc2Cl)cs1)c1cnoc1-c1cncnc1]

| | |
|---|---|
| Acid CAS | 1498038-23-5 |
| Amino CAS | 2150665-06-6 |

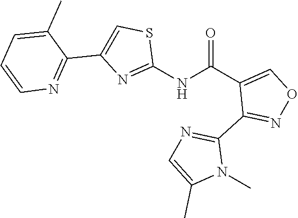 [SMILES: Cc1cccnc1-c1csc(NC(=O)c2conc2-c2ncc(C)n2C)n1]

| | |
|---|---|
| Acid CAS | 2167238-82-4 |
| Amino CAS | 1044269-91-1 |

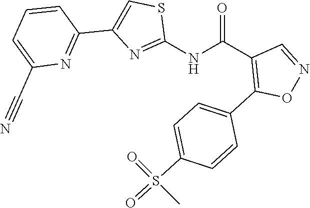 [SMILES: CS(=O)(=O)c1ccc(-c2oncc2C(=O)Nc2nc(-c3cccc(C#N)n3)cs2)cc1]

| | |
|---|---|
| Acid CAS | 1368404-20-9 |
| Amino CAS | 1185317-90-1 |

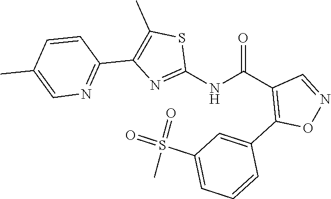 [SMILES: Cc1ccc(-c2nc(NC(=O)c3cnoc3-c3cccc(S(C)(=O)=O)c3)sc2C)nc1]

| | |
|---|---|
| Acid CAS | 2228628-28-0 |
| Amino CAS | 2294596-48-6 |

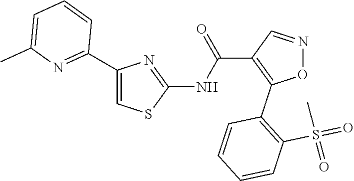 [SMILES: Cc1cccc(-c2csc(NC(=O)c3cnoc3-c3ccccc3S(C)(=O)=O)n2)n1]

| | |
|---|---|
| Acid CAS | 2228267-03-4 |
| Amino CAS | 99358-99-3 |

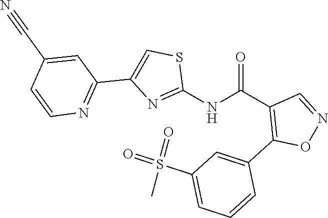 [SMILES: CS(=O)(=O)c1cccc(-c2oncc2C(=O)Nc2nc(-c3cc(C#N)ccn3)cs2)c1]

| | |
|---|---|
| Acid CAS | 2228628-28-0 |
| Amino CAS | 2253868-97-0 |

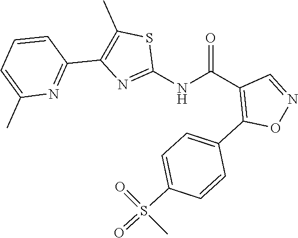 [SMILES: Cc1cccc(-c2nc(NC(=O)c3cnoc3-c3ccc(S(C)(=O)=O)cc3)sc2C)n1]

| | |
|---|---|
| Acid CAS | 1368404-20-9 |
| Amino CAS | 2169472-77-7 |

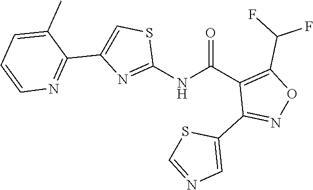 [SMILES: Cc1cccnc1-c1csc(NC(=O)c2c(-c3cncs3)noc2C(F)F)n1]

| | |
|---|---|
| Acid CAS | 2137989-37-6 |
| Amino CAS | 1044269-91-1 |

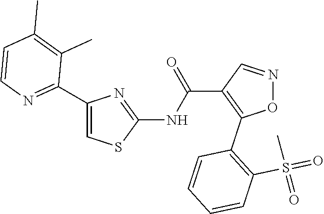 [SMILES: Cc1ccnc(-c2csc(NC(=O)c3cnoc3-c3ccccc3S(C)(=O)=O)n2)c1C]

| | |
|---|---|
| Acid CAS | 2228267-03-4 |
| Amino CAS | 2169166-95-2 |

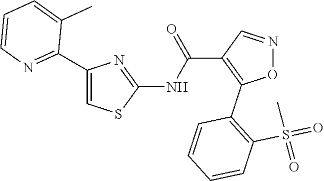 [SMILES: Cc1cccnc1-c1csc(NC(=O)c2cnoc2-c2ccccc2S(C)(=O)=O)n1]

| | |
|---|---|
| Acid CAS | 2228267-03-4 |
| Amino CAS | 1044269-91-1 |

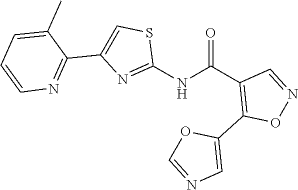 [SMILES: Cc1cccnc1-c1csc(NC(=O)c2cnoc2-c2cnco2)n1]

| | |
|---|---|
| Acid CAS | 1487235-37-6 |
| Amino CAS | 1044269-91-1 |

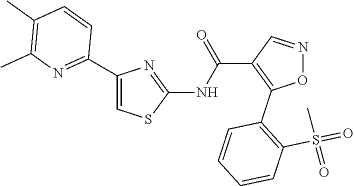 [SMILES: Cc1ccc(-c2csc(NC(=O)c3cnoc3-c3ccccc3S(C)(=O)=O)n2)nc1C]

| | |
|---|---|
| Acid CAS | 2228267-03-4 |
| Amino CAS | 1934459-93-4 |

### Activity in Reporter Assays

All compounds were tested using the human cell line HEK STF293. This cell line carries a Wnt reporter (TCF/LEF1 promoter), which drives expression of the firefly luciferase protein. The level of Wnt activity is directly correlated with the level of luciferase activity (determined using a simple assay). Compounds that inhibit Wnt signaling by reducing luciferase activity in these two cell lines are further tested biochemically. Biochemical confirmation that compounds inhibit Wnt signaling is obtained by immunoblotting for beta-catenin in HEK STF293 cells and demonstrating that its levels are reduced. Compounds were were further tested in a Viability assay using Wnt-dependent HCT116 cell line.

Compounds were prepared as 10 mM stocks for each compound in DMSO. Dilutions were prepared in a 96-well plate in DMSO. The stock dilutions are as follows: 10 mM, 1 mM, 100 μM, 10 μM, 100 nM, and 10 nM. Plates were sealed and stored at −20° C.

HEK STF293 cells were seeded at approximately 25,000-30,000 cells/well in a 96-well (100 μL volume). On the first day, Wnt3a-conditioned media (1:1) were added along with diluted compounds (1:100). For example, for 100 μL of STF293 cells, 100 μL of Wnt3a-conditioned media and 2 μL of drug was added to each well. The final concentrations should therefore be 100 μM, 10 μM, 1 μM, 100 nM, 10 nM, and 1 nM. On the second day, the media was removed and 75 μL of Passive Lysis Buffer (Promega) is added to each well. The plate was shaken at 130 rpm for 15 minutes. For the Steady Glo assay, 45 μL of the lysis was removed and added to a white 96-well plate containing 45 μL/well of Steady Glo solution (Promega). For the Cell Titer assay, 25 μL of the lysis was transferred to a white 96-well plate containing 25 μL/well of Cell Titer solution (Promega). Both Steady Glo and Cell Titer assays were read with a luminescence plate reader. When determining EC50, the Steady Glo values were divided by the Cell Titer values to normalize for cell number.

The control CMV driven cell line assay was performed as recited above for the STF293 assay except that no Wnt3a-conditioned media was added to the plated cells and 1 μL of diluted compound was added instead of 2 μL.

Three concentrations were chosen based on the EC50 curves from the STF293 assay. From the original 10 mM stocks, the following dilutions were prepared in DMSO and stored at −20° C.: 100 μM, 50 μM, and 10 μM.

HEK293 cells were seeded in a 6-well plate at approximately 8.0×10⁵ cells (2 mL per well). On the first day, Wnt3a-conditioned media (1:1) and compounds (1:100) were added to the plated cells. The final concentrations of compounds were 1 μM, 500 nM, and 100 nM. Vehicle (DMSO) and a Wnt3a-conditioned media plus Vehicle samples were also prepared as controls. Lysates were collected (with non-denaturing lysis buffer) after 24 hours incubation, and protein concentrations determined by Bradford Assay. Immunoblotting with an anti-beta-catenin antibody (equivalent amounts of protein/lane for each condition) were subsequently performed to determine beta-catenin levels. HCT116 cells were seeded at 2,500 cells/well in a 96-well dish (Volume: 100 μL/well). On the second day, the 10 mM stock solution of each test compound was thawed at room temperature. Dilutions of the test compound were prepared in DMSO in a V-bottom 96-well plate. The stock compound solution, DMSO-diluted compound solutions, and DMSO were further diluted 500-fold in cell culture media, and 100 μL of the compound- or DMSO-containing cell culture media was added to each well of the cells. The final compound concentrations, therefore, were 10 μM, 3.3.3 μM, 1.11 μM, 370.37 nM, 123.46 nM, 41.15 nM, 13.72 nM, 4.57 nM and zero. The final DMSO concentration was 0.1% in all wells. On the fifth day, 150 μL of media was removed from each well. 50 μL/well Cell Titer Glo reagent (Promega) was added to each well. The plate was shaken at 130 rpm for 15 minutes at room temperature, and the cell viability was determined using a luminescence plate reader.

Results from the HEK STF293 assay disclosed above are shown in Table 3 below. Results from the Viability assay disclosed above are shown in Table 6 below. Potency categories are defined as follows: A: <200 nM, B: 200-500 nM, C: 500-1000 nM, D: 1,000-10,000 nM.

TABLE 6

| Example number | TOPFlash IC₅₀ category | Viability IC₅₀ category |
|---|---|---|
| 1 | A | B |
| 2 | A | B |
| 3 | B | B |
| 4 | A | B |
| 5 | B | B |
| 6 | B | B |
| 7 | B | B |
| 8 | A | A |
| 9 | A | A |
| 10 | A | B |
| 11 | B | B |
| 12 | B | B |
| 13 | A | A |
| 14 | A | B |
| 15 | B | B |
| 16 | B | B |
| 17 | B | B |
| 18 | B | C |
| 19 | B | B |
| 20 | B | B |
| 21 | D | D |
| 22 | B | B |
| 23 | B | B |
| 24 | A | A |
| 25 | B | C |
| 26 | B | A |
| 27 | B | B |
| 28 | B | B |
| 29 | A | A |
| 30 | A | B |
| 31 | A | B |
| 32 | A | B |
| 33 | A | B |
| 34 | D | D |
| 35 | C | D |
| 36 | B | C |
| 37 | A | B |
| 38 | B | C |
| 39 | C | C |
| 40 | B | B |
| 41 | C | C |
| 42 | C | C |
| 43 | A | A |
| 44 | D | C |
| 45 | D | D |
| 46 | C | C |
| 47 | C | B |
| 48 | C | B |
| 49 | B | C |
| 50 | B | B |
| 51 | C | C |
| 52 | C | C |
| 53 | C | C |
| 54 | D | D |
| 55 | B | B |
| 56 | C | C |
| 57 | A | A |
| 58 | A | A |
| 59 | A | A |
| 60 | A | A |
| 61 | A | A |
| 62 | B | A |
| 63 | B | B |
| 64 | C | B |
| 65 | A | A |
| 66 | A | A |
| 67 | B | B |
| 68 | A | A |
| 69 | B | B |
| 70 | C | B |
| 71 | B | B |
| 72 | B | B |
| 73 | A | A |
| 74 | A | A |
| 75 | B | A |
| 76 | A | A |
| 77 | B | C |
| 78 | C | D |

### Examples for Formulae (VIII), (IX), and (X)

### Experimental Procedures and Characterization Data

### General Procedure a (Amide Coupling)

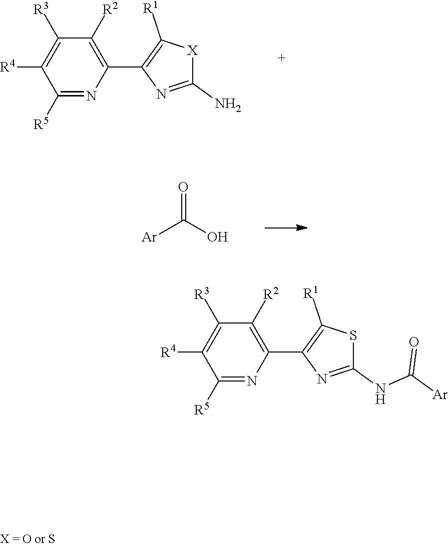

To a stirred solution of an appropriately substituted aromatic carboxylic acid in a suitable organic solvent (e.g., DMF) at room temperature was added 1-2 equivalents of a suitable amide coupling reagent (e.g., HATU) followed by 2-3 equivalents of a suitable organic base (e.g., diisopropylethylamine). A solution of an appropriately substituted aromatic amine in a suitable organic solvent (e.g., DMF) was added to the reaction mixture. The resultant reaction mixture was stirred at room temperature with progress monitored periodically by TLC and/or LCMS. Upon satisfactory conversion of the amine to the desired amide, the reaction mixture was quenched with water and extracted 2-3× with a suitable, water immiscible organic solvent (e.g., EtOAc). The combined organic extracts were washed with aqueous sodium bicarbonate and/or other aqueous media, then dried over a suitable drying agent (e.g., anhydrous MgSO₄). Filtration followed by removal of solvent via rotary evaporation provided a residue that was subsequently purified by normal or reverse phase chromatography to yield the purified amide product.

The following Examples were synthesized using General Procedure A, starting with commercially available or known amine and carboxylic acid building blocks, as shown in Table 7.

TABLE 7

| | | Proton NMR | LHS BB | RHS BB |
|---|---|---|---|---|
| Example | Structure | (400 MHz, DMSO-d6) | CAS | CAS |
| 1 | 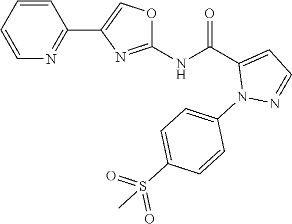 [SMILES: CS(=O)(=O)c1ccc(-n2nccc2C(=O)Nc2nc(-c3ccccn3)co2)cc1] | 12.18 (s, 1H), 8.59-8.57 (m, 1H), 8.46 (s, 1H), 8.05-8.02 (m, 2H), 7.94 (d, J = 2.0 Hz, 1H), 7.91-7.86 (m, 1H), 7.79-7.74 (m, 3H), 7.37-7.32 (m, 2H), 3.30 (s, 3H) | 1014629-84-5 | 681454-61-5 |
| 2 | 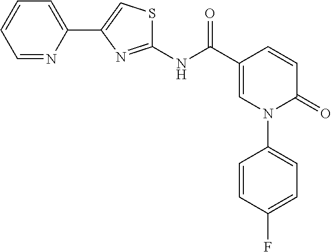 [SMILES: O=C(Nc1nc(-c2ccccn2)cs1)c1ccc(=O)n(-c2ccc(F)cc2)c1] | 8.76 (d, J = 2.7 Hz, 1H), 8.60 (ddd, J = 4.8, 1.8, 0.9 Hz, 1H), 8.07 (dd, J = 9.6, 2.7 Hz, 1H), 7.95 (dt, J = 7.9, 1.2 Hz, 1H), 7.91-7.83 (m, 2H), 7.66-7.56 (m, 2H), 7.47-7.37 (m, 2H), 7.33 (ddd, J = 7.5, 4.7, 1.3 Hz, 1H), 6.61 (d, J = 9.7 Hz, 1H), NH not observed | 30235-26-8 | 1221423-61-5 |
| 3 | 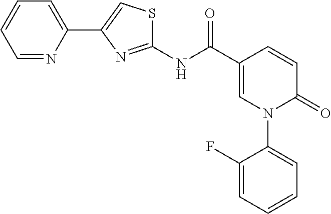 [SMILES: O=C(Nc1nc(-c2ccccn2)cs1)c1ccc(=O)n(-c2ccccc2F)c1] | 8.78 (d, J = 2.7 Hz, 1H), 8.60 (ddd, J = 4.8, 1.8, 0.9 Hz, 1H), 8.11 (dd, J = 9.7, 2.7 Hz, 1H), 7.95 (dt, J = 7.9, 1.2 Hz, 1H), 7.87 (td, J = 7.7, 1.8 Hz, 1H), 7.86 (s, 1H), 7.69-7.55 (m, 2H), 7.48-7.44 (m, 2H), 7.33 (ddd, J = 7.5, 4.8, 1.3 Hz, 1H), 6.64 (d, J = 9.7 Hz, 1H), NH not observed | 30235-26-8 | 1284640-83-0 |
| 4 | 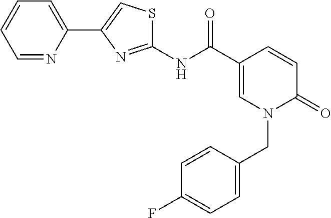 [SMILES: O=C(Nc1nc(-c2ccccn2)cs1)c1ccc(=O)n(Cc2ccc(F)cc2)c1] | 8.90 (d, J = 2.7 Hz, 1H), 8.61 (ddd, J = 4.8, 1.8, 0.9 Hz, 1H), 8.06 (dd, J = 9.6, 2.7 Hz, 1H), 7.99 (dt, J = 7.9, 1.2 Hz, 1H), 7.93-7.85 (m, 1H), 7.86 (s, 1H), 7.51-7.42 (m, 2H), 7.34 (ddd, J = 7.5, 4.8, 1.2 Hz, 1H), 7.25- 7.15 (m, 2H), 6.51 (d, J = 9.6 Hz, 1H), 5.15 (s, 2H), NH not observed | 30235-26-8 | 941869-20-1 |
| 5 | 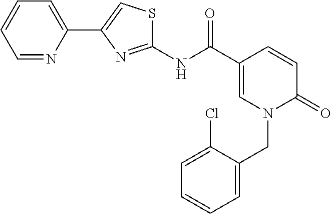 [SMILES: O=C(Nc1nc(-c2ccccn2)cs1)c1ccc(=O)n(Cc2ccccc2Cl)c1] | 8.78 (d, J = 2.7 Hz, 1H), 8.60 (ddd, J = 4.8, 1.8, 0.9 Hz, 1H), 8.15 (dd, J = 9.6, 2.7 Hz, 1H), 8.00-7.83 (m, 3H), 7.56-7.49 (m, 1H), 7.36-7.32 (m, 3H), 7.06-6.99 (m, 1H), 6.56 (d, J = 9.6 Hz, 1H), 5.27 (s, 2H), NH not observed | 30235-26-8 | 4399-77-3 |
| 6 | 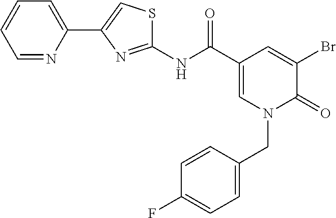 [SMILES: O=C(Nc1nc(-c2ccccn2)cs1)c1cc(Br)c(=O)n(Cc2ccc(F)cc2)c1] | 8.95 (d, J = 2.4 Hz, 1H), 8.64- 8.56 (m, 2H), 7.99 (dt, J = 7.9, 1.1 Hz, 1H), 7.89 (td, J = 7.7, 1.8 Hz, 1H), 7.88 (s, 1H), 7.53- 7.45 (m, 2H), 7.34 (ddd, J = 7.5, 4.8, 1.3 Hz, 1H), 7.28-7.12 (m, 2H), 5.23 (s, 2H), NH not observed | 30235-26-8 | 1128105-65-6 |
| 7 | 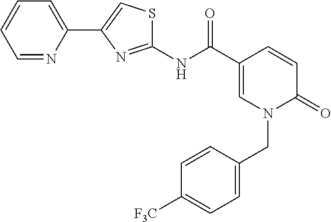 [SMILES: O=C(Nc1nc(-c2ccccn2)cs1)c1ccc(=O)n(Cc2ccc(C(F)(F)F)cc2)c1] | 8.92 (d, J = 2.7 Hz, 1H), 8.61 (ddd, J = 4.8, 1.8, 0.9 Hz, 1H), 8.09 (dd, J = 9.6, 2.7 Hz, 1H), 7.98 (dt, J = 7.9, 1.2 Hz, 1H), 7.93-7.84 (m, 1H), 7.87 (s, 1H), 7.74 (d, J = 8.1 Hz, 2H), 7.58 (d, J = 8.1 Hz, 2H), 7.33 (ddd, J= 7.5, 4.8, 1.3 Hz, 1H), 6.54 (d, J = 9.6 Hz, 1H), 5.27 (s, 2H), NH not observed | 30235-26-8 | 338783-75-8 |
| 8 | 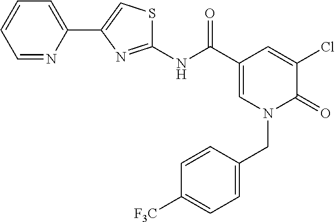 [SMILES: O=C(Nc1nc(-c2ccccn2)cs1)c1cc(Cl)c(=O)n(Cc2ccc(C(F)(F)F)cc2)c1] | 8.94 (d, J = 2.4 Hz, 1H), 8.61 (ddd, J = 4.8, 1.8, 0.9 Hz, 1H), 8.45 (d, J = 2.4 Hz, 1H), 7.98 (dt, J = 7.9, 1.1 Hz, 1H), 7.93- 7.85 (m, 2H), 7.75 (d, J = 8.1 Hz, 2H), 7.60 (d, J = 8.1 Hz, 2H), 7.34 (ddd, J = 7.5, 4.8, 1.3 Hz, 1H), 5.35 (s, 2H), NH not observed | 30235-26-8 | 886761-94-0 |
| 9 | 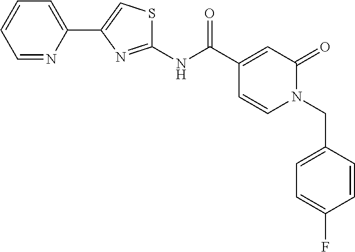 [SMILES: O=C(Nc1nc(-c2ccccn2)cs1)c1ccn(Cc2ccc(F)cc2)c(=O)c1] | 8.61 (ddd, J = 4.8, 1.8, 0.9 Hz, 1H), 8.03-7.96 (m, 2H), 7.92 (s, 1H), 7.89 (td, J = 7.7, 1.8 Hz, 1H), 7.46-7.37 (m, 2H), 7.34 (ddd, J = 7.5, 4.8, 1.2 Hz, 1H), 7.24-7.11 (m, 3H), 6.75 (dd, J= 7.1, 2.0 Hz, 1H), 5.12 (s, 2H), NH not observed | 30235-26-8 | 1368927-27-8 |
| 10 | 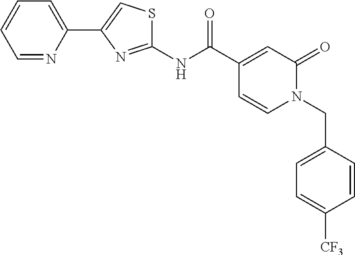 [SMILES: O=C(Nc1nc(-c2ccccn2)cs1)c1ccn(Cc2ccc(C(F)(F)F)cc2)c(=O)c1] | 8.61 (ddd, J = 4.8, 1.8, 0.9 Hz, 1H), 8.06-7.96 (m, 2H), 7.95- 7.85 (m, 2H), 7.73 (d, J = 8.1 Hz, 2H), 7.52 (d, J = 8.0 Hz, 2H), 7.34 (ddd, J = 7.5, 4.8, 1.3 Hz, 1H), 7.17 (d, J = 2.0 Hz, 1H), 6.79 (dd, J = 7.1, 2.0 Hz, 1H), 5.24 (s, 2H), NH not observed | 30235-26-8 | 1556050-28-2 |
| 11 | 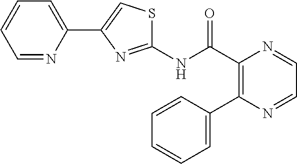 [SMILES: O=C(Nc1nc(-c2ccccn2)cs1)c1nccnc1-c1ccccc1] | 8.93 (d, J = 2.5 Hz, 1H), 8.75 (d, J = 2.5 Hz, 1H), 8.60 (ddd, J= 4.8, 1.8, 1.0 Hz, 1H), 7.95-7.83 (m, 3H), 7.72-7.62 (m, 2H), 7.53-7.42 (m, 3H), 7.33 (ddd, J = 7.3, 4.8, 1.5 Hz, 1H), NH not observed | 30235-26-8 | 2881-85-8 |
| 12 | 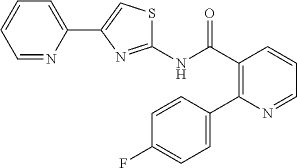 [SMILES: O=C(Nc1nc(-c2ccccn2)cs1)c1cccnc1-c1ccc(F)cc1] | 8.79 (dd, J = 4.8, 1.7 Hz, 1H), 8.59 (dt, J = 4.7, 1.4 Hz, 1H), 8.10 (dd, J = 7.7, 1.7 Hz, 1H), 7.88 (s, 2H), 7.93-7.81 (m, 1H), 7.68-7.58 (m, 2H), 7.52 (dd, J= 7.8, 4.8 Hz, 1H), 7.32 (ddd, J= 6.8, 4.8, 1.8 Hz, 1H), 7.31-7.21 (m, 2H), NH not observed | 30235-26-8 | 101419-78-7 |
| 13 | 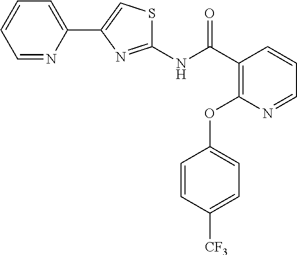 [SMILES: O=C(Nc1nc(-c2ccccn2)cs1)c1cccnc1Oc1ccc(C(F)(F)F)cc1] | 8.60 (ddd, J = 4.8, 1.8, 0.9 Hz, 1H), 8.29 (dd, J = 4.9, 1.9 Hz, 1H), 8.23 (dd, J = 7.5, 1.9 Hz, 1H), 7.98-7.90 (m, 2H), 7.86 (td, J = 7.7, 1.8 Hz, 1H), 7.75- 7.56 (m, 5H), 7.37-7.28 (m, 2H) | 30235-26-8 | 1053982-51-6 |

### Synthesis of Example 14

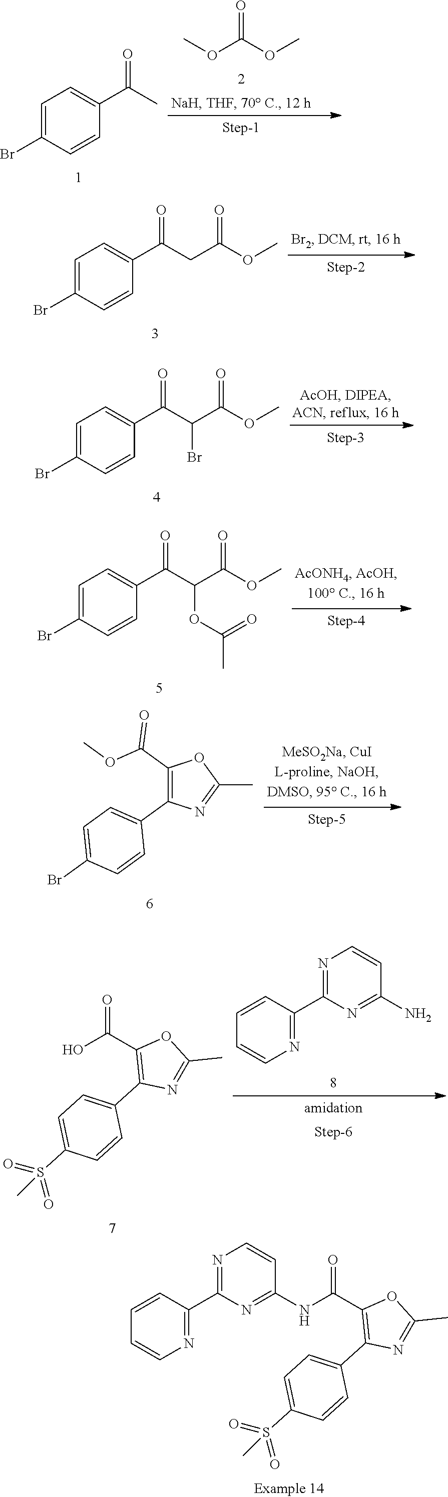

### Step-1: Methyl 3-(4-bromophenyl)-3-oxopropanoate (3)

To a stirred solution of 1-(4-bromophenyl)ethan-1-one (1, 120 g, 602.86 mmol, 1.0 eq) in THF (1200 mL) were added dimethyl carbonate (2, 162.80 g, 1808 mmol, 3.0 eq) and 60% NaH (48.2 g, 1204 mmol, 2.0 eq) at 0° C. The reaction mixture was refluxed for 12 h. After completion of the reaction, the mixture was cooled to RT and poured into ice-cold water and acidified with aq. HCl to pH 2-3 and extracted with EtOAc (3×200 mL), washed with H₂O (200 mL), brine (100 mL). The combined organic layer was dried over Na₂SO₄ and concentrated under reduced pressure to afford the crude compound which was purified through combi-flash column chromatography (10% EtOAc in heptane) to give methyl 3-(4-bromophenyl)-3-oxopropanoate (3, 110 g, 71%) as a yellow color solid. LC-MS: m/z 257.0 [M+H]⁺.

### Step-2: Methyl 2-bromo-3-(4-bromophenyl)-3-oxopropanoate (4)

To a stirred solution of methyl 3-(4-bromophenyl)-3-oxopropanoate (3, 37 g, 144 mmol, 1.0 eq) in DCM (400 mL) was added Br₂ (9.60 mL, 1.3 eq) at 0° C. and stirred at RT for 16 h. After completion of the reaction, solvent was evaporated to dryness and the residue was purified by combi-flash column chromatography (5-8% EtOAc in hexane) to afford methyl 2-bromo-3-(4-bromophenyl)-3-oxopropanoate (4, 25 g, 52%) as a light brown solid. The product was confirmed by LC-MS: m/z 335.10 [M+H]⁺.

### Step-3: Methyl 2-acetoxy-3-(4-bromophenyl)-3-oxopropanoate (5)

To a stirred solution of methyl 2-bromo-3-(4-bromophenyl)-3-oxopropanoate (4, 32 g, 95.23 mmol, 1.0 eq) in ACN (320 mL) was added AcOH (5.71 g, 19 mmol, 0.2 eq) and DIPEA (25 mL, 143.0 mmol, 1.50 eq) at RT and reflux for 16 h. Progress of the reaction was monitored by TLC. After completion of the reaction, solvent was evaporated under rotatory to get the crude compound which was extracted with EtOAc (3×200 mL), washed with H₂O (100 mL), brine and dried over Na₂SO₄. The combined organic layer was concentrated in vacuo to get the crude methyl 2-acetoxy-3-(4-bromophenyl)-3-oxopropanoate (5, 30 g crude) as a light brown oil which was used as such into next step without purification. ¹H NMR (400 MHz, DMSO-d6): δ7.97 (d, J=8.80 Hz, 2H), 7.68 (d, J=8.80 Hz, 2H), 3.84 (s, 3H), 2.55 (s, 3H). LC-MS: m/z 314.30 [M+H]⁺.

### Step-4: Methyl 4-(4-bromophenyl)-2-methyloxazole-5-carboxylate (6)

To a stirred solution of methyl 2-acetoxy-3-(4-bromophenyl)-3-oxopropanoate (5, 30.40 g, 96.50 mmol, 1.0 eq) in AcOH (300 mL) was added NH₄OAc (5.16 g, 483.35 mmol, 5.0 eq) at RT. The resulting reaction mixture was heated at 100° C. for 16 h. Progress of the reaction was monitored by TLC. After completion of reaction, the excess acetic acid was distilled out and the resulting crude was poured into ice-cold water (500 mL) and extracted with EtOAc (3×200 mL), washed with H₂O (3×100 mL), brine (150 mL) and dried over sodium sulfate, filtered and concentrated under vacuum to get crude material which was purified through combi-flash column chromatography (15% EtOAc in hexane) to afford methyl 4-(4-bromophenyl)-2-methyloxazole-5-carboxylate (6, 6.50 g, 23%) as a light yellow liquid. ¹H NMR (400 MHz, DMSO-d6): δ 7.98 (d, J=8.80 Hz, 2H), 7.68 (d, J=8.80 Hz, 2H), 3.84 (s, 3H), 2.54 (s, 3H); LC-MS: m/z 296.0 [M+H]⁺.

### Step-5: 2-Methyl-4-(4-(methylsulfonyl)phenyl)oxazole-5-carboxylic acid (7)

To a stirred solution of methyl 4-(4-bromophenyl)-2-methyloxazole-5-carboxylate (6, 2.0 g, 6.75 mmol, 1.0 eq.) in anhydrous DMSO (20 mL) was added NaOH (0.66 g, 16.2 mmol, 2.4 eq), MeSO₂Na (1.83 g, 15.53 mmol, 2.3 eq) followed by L-proline (1.5 g, 13.53 mmol, 2.0 eq) at RT. The reaction mixture was degassed with argon for 15 min, then CuI (1.41 g, 47.42 mmol, 1.10 eq.) was added and the resulting reaction mixture was stirred at 95° C. for 12 h. Progress of reaction was monitored by TLC and LCMS. The reaction mixture was diluted with cold H₂O, acidified with 1N HCl to bring pH-2 and extracted with 10% MeOH in DCM. The combined organic layer was washed with brine solution, dried over Na₂SO₄ and concentrated under vacuum to afford 2-methyl-4-(4-(methylsulfonyl)phenyl)oxazole-5-carboxylic acid, (7) (2.20 g crude) which was used as such into next step without further purification. ¹H NMR (400 MHz, DMSO-d6): δ 13.80 (br s, 1H), 8.25 (d, J=8.80 Hz, 2H), 8.01 (d, J=8.40 Hz, 2H), 3.26 (s, 3H), 2.55 (s, 3H); LC-MS: m/z 282.20 [M+H]⁺.

### Step-6: Example 14

In line with General Procedure A, compound 7 was coupled with 2-(pyridin-2-yl)-pyrimidin-4-amine (8) to afford Example 14. ¹H NMR (400 MHz, DMSO-d₆) δ 11.22 (br s, 1H), 8.93 (d, J=4.89 Hz, 1H), 8.77-8.79 (m, 1H), 8.50 (d, J=7.2 Hz, 1H), 8.35 (d, J=8.31 Hz, 2H), 8.13-8.21 (m, 2H), 8.04 (d, J=7.6 Hz, 2H), 7.66-7.68 (m, 1H), 3.27 (s, 3H), 2.63 (s, 3H); LC-MS: m/z 436.2 [M+H]⁺; HPLC: 99.6%.

### Synthesis of Example 15

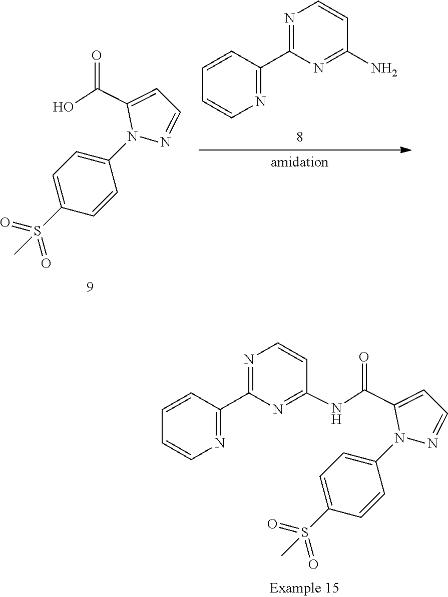

In line with General Procedure A, compound 9 was coupled with 2-(pyridin-2-yl)-pyrimidin-4-amine (8) to afford Example 15. ¹H NMR (400 MHz, DMSO-d₆) δ 11.81 (s, 1H), 8.86 (d, J=5.8 Hz, 1H), 8.77 (d, J=4.0 Hz, 1H), 8.43 (d, J=7.8 Hz, 1H), 8.11-8.02 (m, 4H), 7.94 (d, J=2.0 Hz, 1H), 7.80-7.77 (m, 2H), 7.65-7.61 (m, 1H), 7.46 (d, J=2.0 Hz, 1H), 3.29 (s, 3H); LC-MS: m/z 421.2 [M+H]⁺; HPLC: 98.1%.

### Synthesis of Example 16

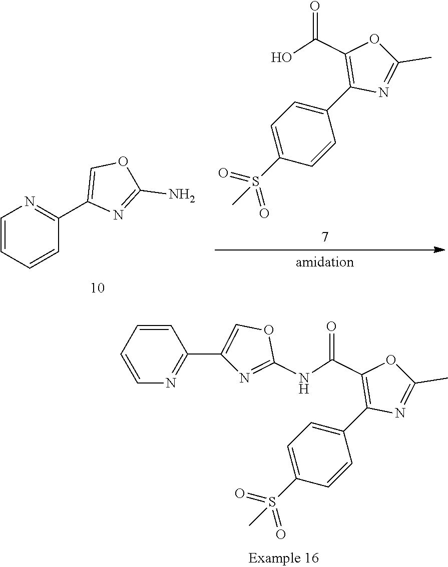 [SMILES: Nc1nc(-c2ccccn2)co1 ; Cc1nc(-c2ccc(S(C)(=O)=O)cc2)c(C(=O)O)o1 ; Cc1nc(-c2ccc(S(C)(=O)=O)cc2)c(C(=O)Nc2nc(-c3ccccn3)co2)o1]

In line with General Procedure A, 2-methyl-4-(4-(methylsulfonyl)phenyl)oxazole-5-carboxylic acid (7) was coupled with 4-(pyridin-2-yl)-oxazol-2-amine (10; CAS #1014629-84-5) to afford Example 16. ¹H NMR (400 MHz, DMSO-d₆) δ 11.96 (br s, 1H), 8.59-8.61 (m, 1H), 8.53 (s, 1H), 8.37-8.41 (m, 2H), 8.02-8.04 (m, 2H), 7.90-7.95 (m, 1H), 7.81 (d, J=7.8 Hz, 1H), 7.36-7.39 (m, 1H), 3.27 (s, 3H), 2.62 (s, 3H); LC-MS: m/z 425.0 [M+H]⁺; HPLC: 99.5%.

### Synthesis of Example 17

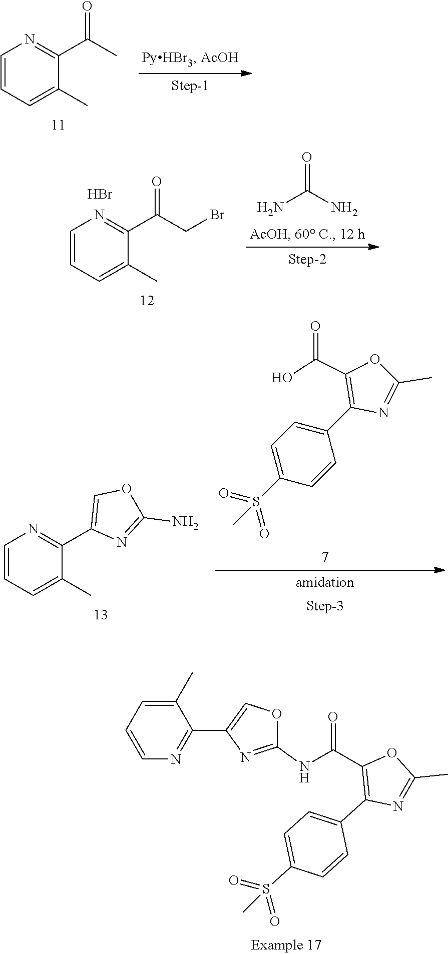

### Step-1: 2-Bromo-1-(3-methylpyridin-2-yl)ethan-1-one HBr salt (12)

To a stirred solution of 1-(3-methylpyridin-2-yl)ethan-1-one (11, 50 g, 369.9 mmol, 1 eq) in acetic acid (200 mL) was added 30% HBr in acetic acid (50.0 mL) at 0° C. within 15 min followed by the addition of Py.HBr₃ (118.08 g, 369.9 mmol, 1.0 eq), in portion wise at same temperature. The mixture was stirred at RT for 16 h. The reaction mix was concentrated under reduced pressure to get brown color solid which was washed with heptane and dried under reduced pressure to afford 12 (55 g, 51%) as brown color solid which was as such into next step without purification.

### Step-2: 4-(3-methylpyridin-2-yl)oxazol-2-amine (13)

To a stirred solution of 2-bromo-1-(3-methylpyridin-2-yl)ethan-1-one HBr (12, 30 g, 101.70 mmol, 1.0 eq) in AcOH (200 mL) was added urea (15.27 g, 254.20 mmol, 2.5 eq) at RT and heated at 60° C. for 12 h. After the completion of reaction, excess of AcOH was concentrated and treated with saturated NaHCO₃ and extracted with 10% MeOH in DCM. The combined organic layer was washed with brine, dried over Na₂SO₄ and concentrated. The crude compound was purified through Silica gel column chromatography using 3-5% MeOH in DCM to afford 4-(3-methylpyridin-2-yl)oxazol-2-amine (4.5 g, 26%) as a dark brown solid. LC-MS: m/z 176.2 [M+H]⁺.

### Step-3: Example 17

In line with General Procedure A, 2-methyl-4-(4-(methylsulfonyl)phenyl)oxazole-5-carboxylic acid (7) was coupled with 4-(3-methylpyridin-2-yl)oxazol-2-amine (13) to afford Example 17. ¹H NMR (400 MHz, DMSO-d₆) δ 11.92 (br s, 1H), 8.51 (d, J=4.8 Hz, 1H), 8.49 10 (s, 1H), 8.38 (d, J=8.8 Hz, 2H), 8.03 (d, J=8.6 Hz, 2H), 7.85 (d, J=7.1 Hz, 1H), 7.39-7.42 (m, 1H), 3.27 (s, 3H), 2.62 (s, 3H), 2.57 (s, 3H); LC-MS: m/z 438.9 [M+H]⁺; HPLC: 98.0%.

### Synthesis of Example 18

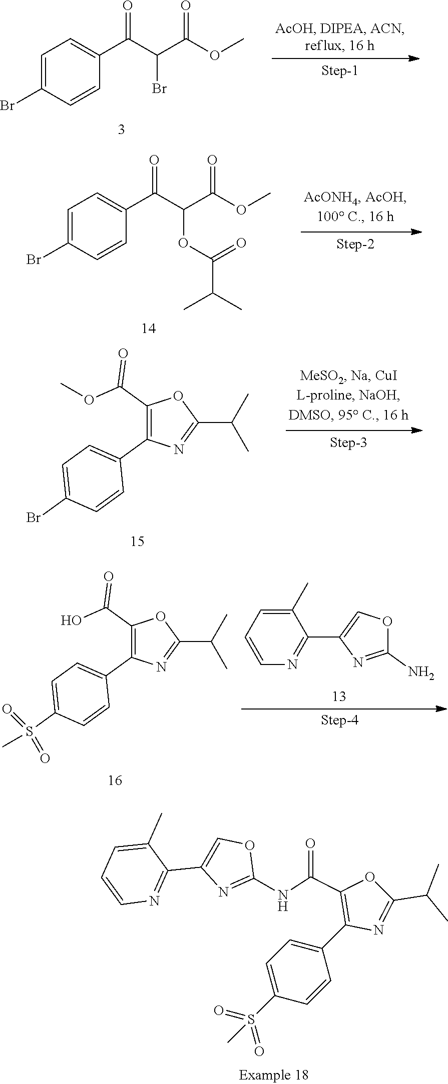

### Step-1: Methyl 3-(4-bromophenyl)-2-(isobutyryloxy)-3-oxopropanoate (14)

To a stirred solution of methyl 2-bromo-3-(4-bromophenyl)-3-oxopropanoate (3, 13 g, 38 mmol, 1.0 eq) in ACN (130 mL) was added AcOH (3.52 mL, 38 mmol, 1.0 eq) and DIPEA (10 mL, 58 mmol, 1.5 eq) at RT and heated at 90° C. for 12 h. Progress of the reaction was monitored by TLC. After completion of the reaction, solvent was evaporated under rotatory to get the crude compound which was extracted with EtOAc (3×200 mL), washed with H₂O (100 mL), brine and dried over Na₂SO₄. The combined organic layer was concentrated in vacuo to get methyl 3-(4-bromophenyl)-2-(isobutyryloxy)-3-oxopropanoate (14, 12.8 g crude) which was used as such into next step without purification. LC-MS: m/z 343.0 [M+H]⁺.

### Step-2: Methyl 4-(4-bromophenyl)-2-isopropyloxazole-5-carboxylate (15)

To a stirred solution of methyl 3-(4-bromophenyl)-2-(isobutyryloxy)-3-oxopropanoate (14, 12.80 g, 37 mmol, 1.0 eq) in AcOH (130 mL) was added NH₄OAc (14.3 g, 186 mmol, 5.0 eq) at RT. The resulting reaction mixture was heated at 100° C. for 12 h. Progress of the reaction was monitored by TLC. After completion of reaction, the excess of acetic acid was distilled out and the resulting crude was poured into ice-cold water (500 mL) and extracted with EtOAc (3×200 mL), washed with saturated NaHCO₃ (3×100 mL), brine (150 mL), dried over sodium sulfate and concentrated under vacuum to get crude material which was purified through combi-flash column chromatography (20% EtOAc in hexane) to afford methyl 4-(4-bromophenyl)-2-isopropyloxazole-5-carboxylate (15, 7.65 g, 63%) as a light brown liquid. ¹H NMR (400 MHz, DMSO-d6): δ 7.97 (d, J=8.8 Hz, 2H), 7.66 (d, J=6.0 Hz, 2H), 3.83 (s, 3H), 3.22-3.18 (m, 1H), 1.33 (d, J=6.80 Hz, 6H); LC-MS: m/z 324.0 [M+H]⁺.

### Step-3: 2-Isopropyl-4-(4-(methylsulfonyl)phenyl)oxazole-5-carboxylic acid (16)

To a stirred solution of methyl 4-(4-bromophenyl)-2-isopropyloxazole-5-carboxylate (15, 2.0 g, 6.75 mmol, 1.0 eq) in anhydrous DMSO (20 mL) was added NaOH (0.66 g, 16.2 mmol, 2.4 eq), MeSO₂Na (1.83 g, 15.53 mmol, 2.3 eq) followed by L-proline (1.5 g, 13.53 mmol, 2.0 eq) at RT. The reaction mixture was degassed with argon for 15 min, then CuI (1.41 g, 47.42 mmol, 1.10 eq) was added and the resulting reaction mixture was stirred at 95° C. for 12 h. Progress of reaction was monitored by TLC and LCMS. The reaction mixture was diluted with cold H₂O, acidified with 1N HCl to bring pH-2 and extracted with 10% MeOH in DCM. The combined organic layer was washed with brine solution, dried over Na₂SO₄ and concentrated under vacuum to get 2-isopropyl-4-(4-(methylsulfonyl)phenyl)oxazole-5-carboxylic acid (16, 0.9 g, 47%) the crude compound which was used as such into next step without any purification. LC-MS: m/z 310.15 [M+H]⁺.

### Step-3: Example 18

In line with General Procedure A, 2-isopropyl-4-(4-(methylsulfonyl)phenyl)oxazole-5-carboxylic acid (16) was coupled with 4-(3-methylpyridin-2-yl)oxazol-2-amine (13) to afford Example 18. ¹H NMR (400 MHz, DMSO-d₆) δ 8.50 (s, 2H), 8.38 (d, J=8.4 Hz, 2H), 8.03 (d, J=8.8 Hz, 2H), 7.83-7.88 (m, 1H), 7.41 (m, 1H), 3.26 (s, 3H), 2.66 (s, 1H), 2.57 (s, 3H), 1.42 (s, 6H), NH not observed; LC-MS: m/z 467.5 [M+H]⁺; HPLC: 98.4%.

### Synthesis of Example 19

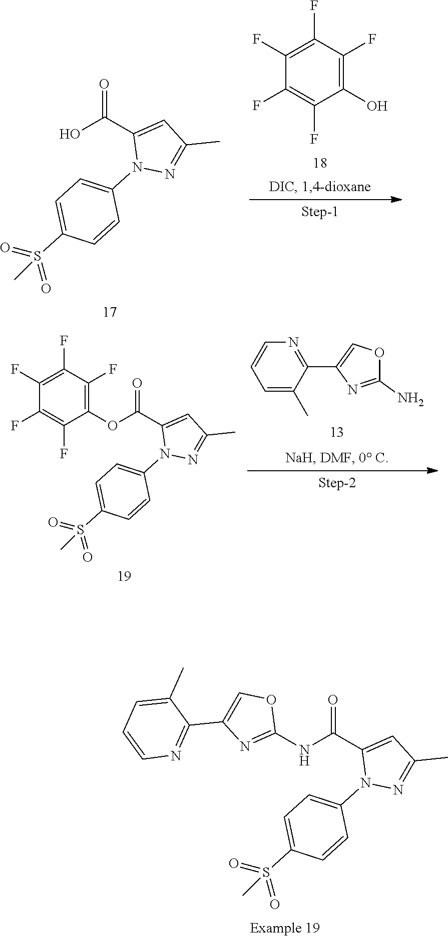 [SMILES: Cc1cc(C(=O)O)n(-c2ccc(S(C)(=O)=O)cc2)n1 ; Oc1c(F)c(F)c(F)c(F)c1F ; Cc1cc(C(=O)Oc2c(F)c(F)c(F)c(F)c2F)n(-c2ccc(S(C)(=O)=O)cc2)n1 ; Cc1cccnc1-c1coc(N)n1 ; Cc1cc(C(=O)Nc2nc(-c3ncccc3C)co2)n(-c2ccc(S(C)(=O)=O)cc2)n1]

### Step-1: Perfluorophenyl 3-methyl-1-(4-(methylsulfonyl)phenyl)-1H-pyrazole-5-carboxylate (19)

To a solution of compound (17, 0.7 g, 2.50 mmol) in 1,4-dioxane (50 mL) was added N, N-diisopropylcarbodiimide (0.472 g, 3.75 mmol) and the reaction mixture was stirred at 0° C. for 10 min. Pentafluorophenol (18, 0.92 g, 5.0 eq) was added and the reaction mixture was allowed to warm to room temperature and stirred overnight. The progress of reaction was monitored by TLC and LCMS. After the completion of reaction, the mixture was concentrated, the residue diluted with ethyl acetate and washed with water, brine solution and dried over Na₂SO₄. The combined organic layer was concentrated on rotavap and the resulting crude compound was purified by silica gel column chromatography (20% EtOAc in Hexane) to afford perfluorophenyl 3-methyl-1-(4-(methyl sulfonyl)phenyl)-1H-pyrazole-5-carboxylate (19, 0.85 g, 76%) as an off-white solid. ¹H NMR (400 MHz, DMSO-d₆): δ 8.04 (d, J=8.0 Hz, 2H), 7.85 (d, J=8.40 Hz, 2H), 7.43 (s, 1H), 3.31 (s, 3H), 2.37 (s, 3H); LC-MS: m/z 447.0 [M+H]⁺.

### Step-2: Example 19

To a solution of 4-(3-methylpyridin-2-yl)oxazol-2-amine (13, 0.286 g, 1.63 mmol) in dry THF (50 mL) was added 60% NaH (98 mg, 2.44 mmol) under nitrogen atmosphere. The reaction mixture was stirred for 10 min at the same temperature. Then, a solution of perfluorophenyl 3-methyl-1-(4-(methylsulfonyl)phenyl)-1H-pyrazole-5-carboxylate (19, 0.8 g, 1.79 mmol) in dry THE was added to the reaction mixture and stirred at room temperature for 1 h. Progress of the reaction was monitored by TLC and LCMS. After the completion of reaction, the mixture was cooled to 0° C. and quenched with ice cold water and extracted with ethyl acetate, washed with brine and dried over Na₂SO₄. The combined organic layer was concentrated under rotatory and the resulting crude compound was purified through reversed phase prep-HPLC to afford 3-methyl-N-(4-(3-methylpyridin-2-yl)oxazol-2-yl)-1-(4-(methylsulfonyl)phenyl)-1H-pyrazole-5-carboxamide (Example 19, 0.21 g, 27%) as an off-white solid. ¹H NMR (400 MHz, DMSO-d₆): δ 12.40 (br s, 1H), 8.49-8.45 (m, 2H), 8.03-8.04 (m, 2H), 7.88-7.86 (m, 1H), 7.73 (d, J=8.80 Hz, 2H), 7.43-7.40 (m, 1H), 7.10 (s, 1H), 3.29 (s, 3H), 2.54 (s, 3H), 2.32 (s, 3H); 438.20 [M+H]⁺; LC-MS: m/z 438.2 [M+H]⁺. HPLC: 97.2%.

### Synthesis of Example 20

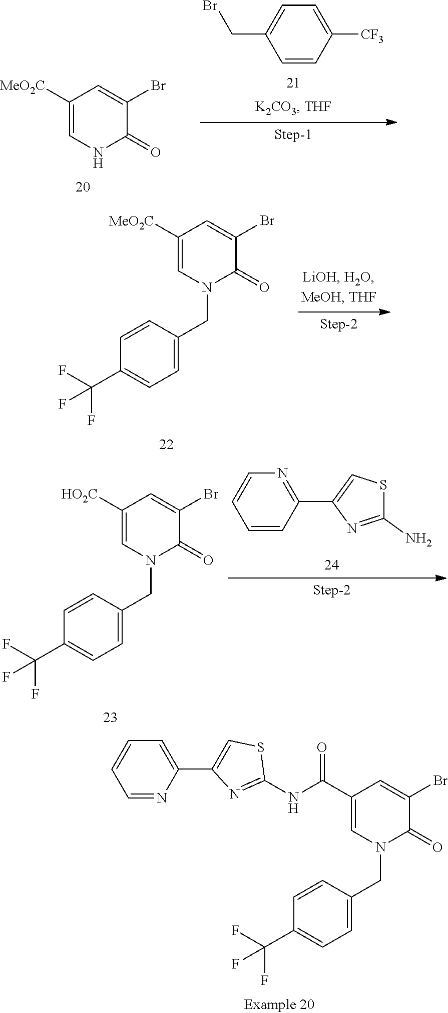

### Step-1: Methyl 5-bromo-6-oxo-1-(4-(trifluoro-methyl)-benzyl)-1,6-dihydro-pyridine-3-carboxylate (22)

To a solution of methyl 5-bromo-6-oxo-1,6-dihydro-pyridine-3-carboxylate (20) (800 mg, 3.45 mmol, 1 eq) in THF (5 mL) at room temperature was added potassium carbonate (714 mg, 5.17 mmol, 1.5 eq) and 4-(trifluoromethyl)benzyl bromide (21) (906 mg, 3.79 mmol, 1.1 eq). The reaction mixture was stirred for 16 hr then quenched with water (20 mL) and extracted with EtOAc (3×20 mL). The combined organic extracts were washed with water (50 mL) and brine (50 mL) and dried over anhydrous magnesium sulfate then evaporated to dryness to afford crude 22 (95% purity) which was used as such in the next step.

### Step-2: 5-bromo-6-oxo-1-(4-(trifluoro-methyl)-benzyl)-1,6-dihydro-pyridine-3-carboxylic acid (22)

To a stirred solution of crude 22 (987 mg, 1 eq) in THE (11.4 mL) at room temperature was added dropwise a solution of 1M LiGH (13.3 mL, 4 eq). MeOH (3.4 mL) was then added at the reaction mixture was stirred overnight then cooled in an ice bath and acidified to pH 3 with dropwise addition of 1M HCl. The resultant yellow precipitate was isolated by filtration, washed with water and dried to afford 23 (800 mg) as a yellow solid, which was used directly in the next step.

### Step-3: Example 20

In line with General Procedure A, 5-bromo-6-oxo-1-(4-(trifluoro-methyl)-benzyl)-1,6-dihydro-pyridine-3-carboxylic acid (22) (376 mg, 1 mmol, 1 eq) was coupled with 4-(pyridin-2-yl)-thiazol-2-amine (24; CAS #30235-26-8) (196 mg, 1.1 mmol, 1.1 eq) to afford Example 20 (291 mg). ¹H NMR (400 MHz, DMSO-d₆) δ 8.98 (d, J=2.4 Hz, 1H), 8.64-8.58 (m, 2H), 7.98 (dt, J=7.9, 1.2 Hz, 1H), 7.93-7.85 (m, 2H), 7.75 (d, J=8.1 Hz, 2H), 7.60 (d, J=8.1 Hz, 2H), 7.34 (ddd, J=7.5, 4.8, 1.3 Hz, 1H), 5.35 (s, 2H), NH not observed; LC-MS: m/z 535.0 [M+H]⁺.

The following Examples can be synthesized using General Procedure A, starting with commercially available or known amine and carboxylic acid building blocks. CAS Registry numbers for each amine and carboxylic acid block is indicated in the Table 8.

TABLE 8 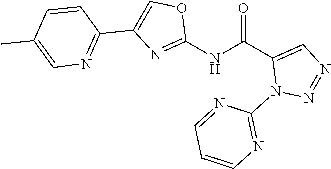 [SMILES: Cc1ccc(-c2coc(NC(=O)c3cnnn3-c3ncccn3)n2)nc1] [symbol]
2946657-14-1 [symbol] 2752153-86-7 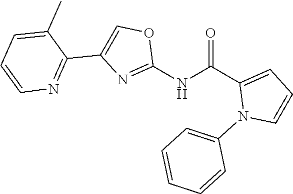 [SMILES: Cc1cccnc1-c1coc(NC(=O)c2cccn2-c2ccccc2)n1] [symbol]
described above [symbol] 78540-03-1 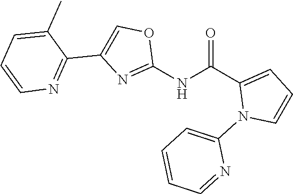 [SMILES: Cc1cccnc1-c1coc(NC(=O)c2cccn2-c2ccccn2)n1] [symbol]
described above [symbol] 896036-05-8 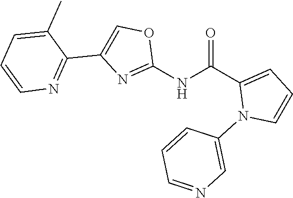 [SMILES: Cc1cccnc1-c1coc(NC(=O)c2cccn2-c2cccnc2)n1] [symbol]
described above [symbol] 896035-98-6 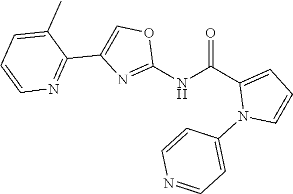 [SMILES: Cc1cccnc1-c1coc(NC(=O)c2cccn2-c2ccncc2)n1] [symbol]
described above [symbol] 1188434-76-5 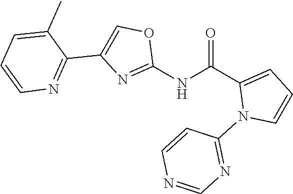 [SMILES: Cc1cccnc1-c1coc(NC(=O)c2cccn2-c2ccncn2)n1] [symbol]
described above [symbol] 1780304-10-0 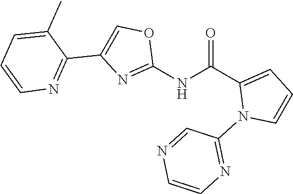 [SMILES: Cc1cccnc1-c1coc(NC(=O)c2cccn2-c2cnccn2)n1] [symbol]
described above [symbol] 896049-56-2 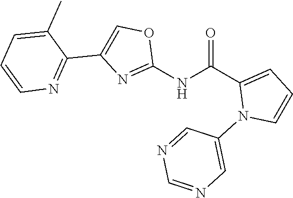 [SMILES: Cc1cccnc1-c1coc(NC(=O)c2cccn2-c2cncnc2)n1] [symbol]
described above [symbol] 1699083-36-7 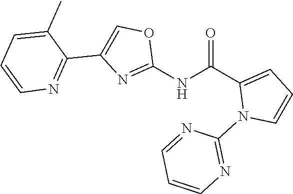 [SMILES: Cc1cccnc1-c1coc(NC(=O)c2cccn2-c2ncccn2)n1] [symbol]
described above [symbol] 896049-60-8 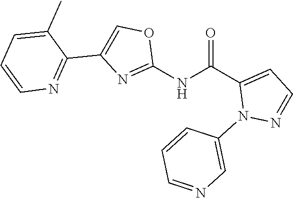 [SMILES: Cc1cccnc1-c1coc(NC(=O)c2ccnn2-c2cccnc2)n1] [symbol]
described above [symbol] 1884400-80-9 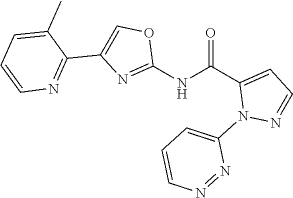 [SMILES: Cc1cccnc1-c1coc(NC(=O)c2ccnn2-c2cccnn2)n1] [symbol]
described above [symbol] 1935682-49-7 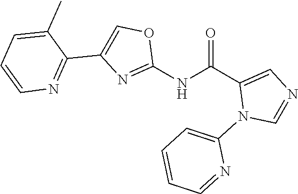 [SMILES: Cc1cccnc1-c1coc(NC(=O)c2cncn2-c2ccccn2)n1] [symbol]
described above [symbol] 1779128-15-2 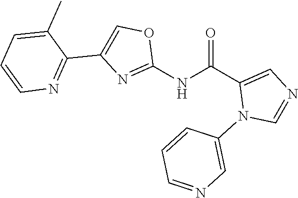 [SMILES: Cc1cccnc1-c1coc(NC(=O)c2cncn2-c2cccnc2)n1] [symbol]
described above [symbol] 1197398-67-6 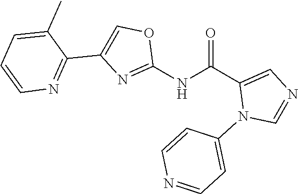 [SMILES: Cc1cccnc1-c1coc(NC(=O)c2cncn2-c2ccncc2)n1] [symbol]
described above [symbol] 1439900-49-8 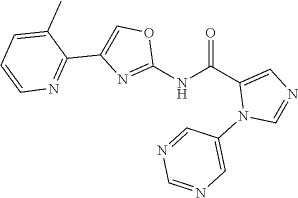 [SMILES: Cc1cccnc1-c1coc(NC(=O)c2cncn2-c2cncnc2)n1] [symbol]
described above [symbol] 1523297-19-9 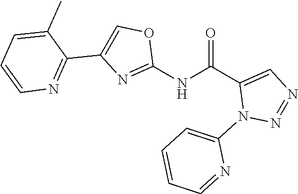 [SMILES: Cc1cccnc1-c1coc(NC(=O)c2cnnn2-c2ccccn2)n1] [symbol]
described above [symbol] 1480445-52-0 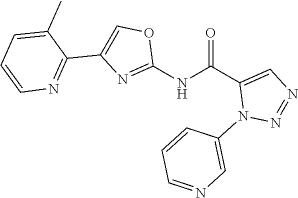 [SMILES: Cc1cccnc1-c1coc(NC(=O)c2cnnn2-c2cccnc2)n1] [symbol]
described above [symbol] 1478271-10-1 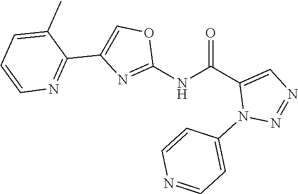 [SMILES: Cc1cccnc1-c1coc(NC(=O)c2cnnn2-c2ccncc2)n1] [symbol]
described above [symbol] 1525417-94-0 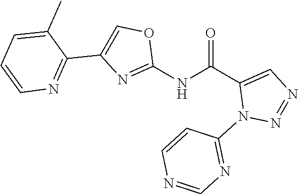 [SMILES: Cc1cccnc1-c1coc(NC(=O)c2cnnn2-c2ccncn2)n1] [symbol]
described above [symbol] 1482934-49-5 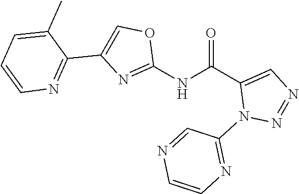 [SMILES: Cc1cccnc1-c1coc(NC(=O)c2cnnn2-c2cnccn2)n1] [symbol]
described above [symbol] 2946657-15-2 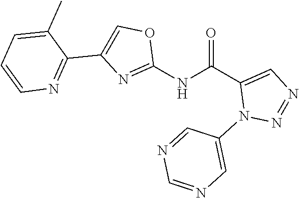 [SMILES: Cc1cccnc1-c1coc(NC(=O)c2cnnn2-c2cncnc2)n1] [symbol]
described above [symbol] 1478093-94-5 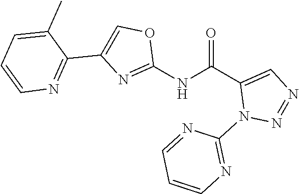 [SMILES: Cc1cccnc1-c1coc(NC(=O)c2cnnn2-c2ncccn2)n1] [symbol]
described above [symbol] 2946657-141 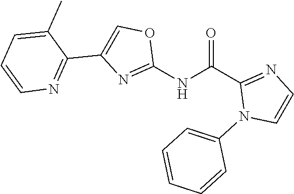 [SMILES: Cc1cccnc1-c1coc(NC(=O)c2nccn2-c2ccccc2)n1] [symbol]
described above [symbol] 518492-57-2 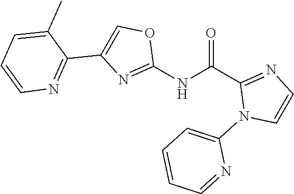 [SMILES: Cc1cccnc1-c1coc(NC(=O)c2nccn2-c2ccccn2)n1] [symbol]
described above [symbol] 1780304-58-6 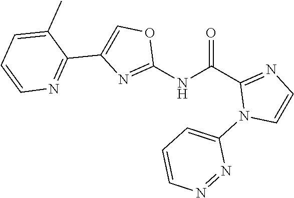 [SMILES: Cc1cccnc1-c1coc(NC(=O)c2nccn2-c2cccnn2)n1] [symbol]
described above [symbol] 1935963-11-3 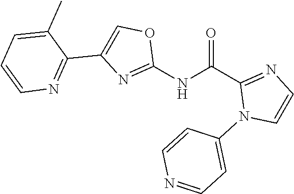 [SMILES: Cc1cccnc1-c1coc(NC(=O)c2nccn2-c2ccncc2)n1] [symbol]
described above [symbol] 1503339-47-6 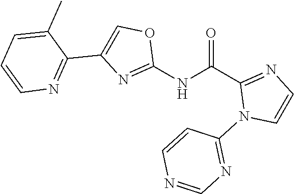 [SMILES: Cc1cccnc1-c1coc(NC(=O)c2nccn2-c2ccncn2)n1] [symbol]
described above [symbol] 1936002-41-3 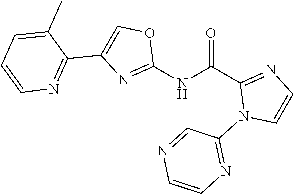 [SMILES: Cc1cccnc1-c1coc(NC(=O)c2nccn2-c2cnccn2)n1] [symbol]
described above [symbol] 1936883-80-5 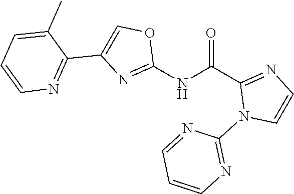 [SMILES: Cc1cccnc1-c1coc(NC(=O)c2nccn2-c2ncccn2)n1] [symbol]
described above [symbol] 1934774-12-5 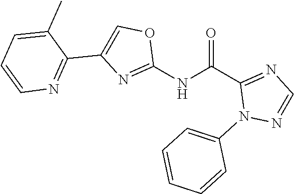 [SMILES: Cc1cccnc1-c1coc(NC(=O)c2ncnn2-c2ccccc2)n1] [symbol]
described above [symbol] 500865-95-2 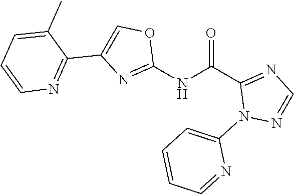 [SMILES: Cc1cccnc1-c1coc(NC(=O)c2ncnn2-c2ccccn2)n1] [symbol]
described above [symbol] 2169404-06-2 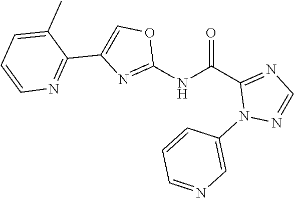 [SMILES: Cc1cccnc1-c1coc(NC(=O)c2ncnn2-c2cccnc2)n1] [symbol]
described above [symbol] 2167545-89-1 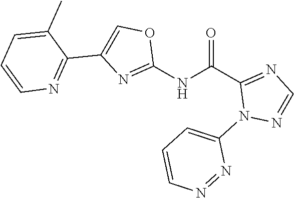 [SMILES: Cc1cccnc1-c1coc(NC(=O)c2ncnn2-c2cccnn2)n1] [symbol]
described above [symbol] 2946657-04-9 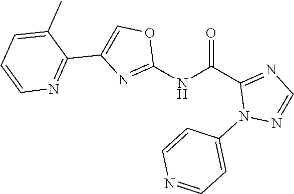 [SMILES: Cc1cccnc1-c1coc(NC(=O)c2ncnn2-c2ccncc2)n1] [symbol]
described above [symbol] 1934935-63-3 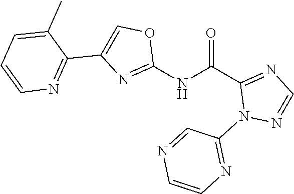 [SMILES: Cc1cccnc1-c1coc(NC(=O)c2ncnn2-c2cnccn2)n1] [symbol]
described above [symbol] 2948657-01-5 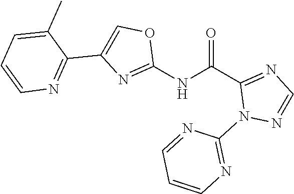 [SMILES: Cc1cccnc1-c1coc(NC(=O)c2ncnn2-c2ncccn2)n1] [symbol]
described above [symbol] 2741044-70-0 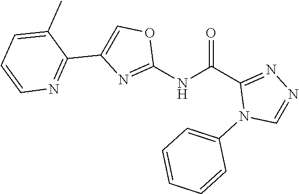 [SMILES: Cc1cccnc1-c1coc(NC(=O)c2nncn2-c2ccccc2)n1] [symbol]
described above [symbol] 1526547-27-2 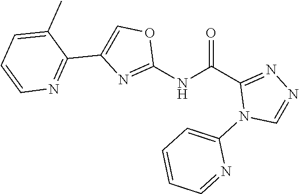 [SMILES: Cc1cccnc1-c1coc(NC(=O)c2nncn2-c2ccccn2)n1] [symbol]
described above [symbol] 2167196-30-5 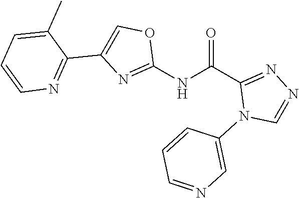 [SMILES: Cc1cccnc1-c1coc(NC(=O)c2nncn2-c2cccnc2)n1] [symbol]
described above [symbol] 2137878-13-6 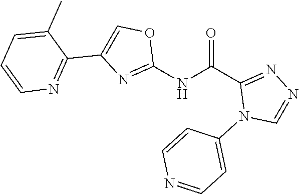 [SMILES: Cc1cccnc1-c1coc(NC(=O)c2nncn2-c2ccncc2)n1] [symbol]
described above [symbol] 2168395-35-3 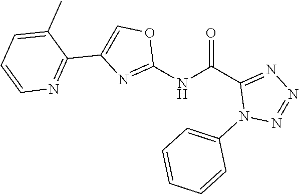 [SMILES: Cc1cccnc1-c1coc(NC(=O)c2nnnn2-c2ccccc2)n1] [symbol]
described above [symbol] 214980-86-6 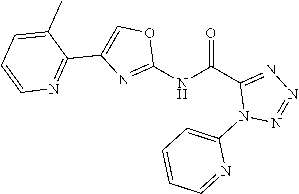 [SMILES: Cc1cccnc1-c1coc(NC(=O)c2nnnn2-c2ccccn2)n1] [symbol]
described above [symbol] 1903206-04-1 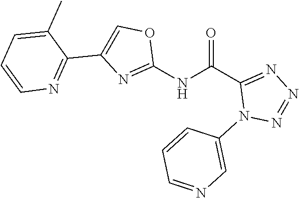 [SMILES: Cc1cccnc1-c1coc(NC(=O)c2nnnn2-c2cccnc2)n1] [symbol]
described above [symbol] 1903020-36-9 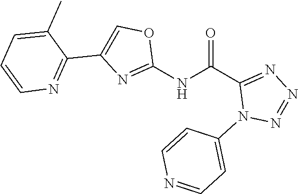 [SMILES: Cc1cccnc1-c1coc(NC(=O)c2nnnn2-c2ccncc2)n1] [symbol]
described above [symbol] 1903870-12-1 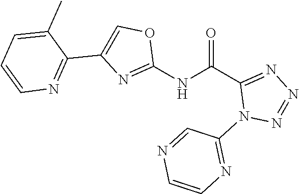 [SMILES: Cc1cccnc1-c1coc(NC(=O)c2nnnn2-c2cnccn2)n1] [symbol]
described above [symbol] 2946657-12-9 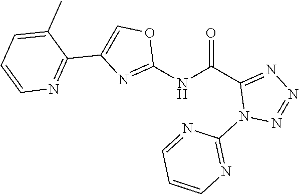 [SMILES: Cc1cccnc1-c1coc(NC(=O)c2nnnn2-c2ncccn2)n1] [symbol]
described above [symbol] 2946657-11-8 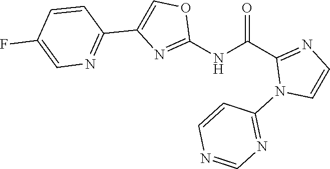 [SMILES: O=C(Nc1nc(-c2ccc(F)cn2)co1)c1nccn1-c1ccncn1] [symbol]
1936002-41-3 [symbol] 1544907-72-3 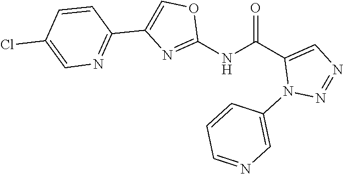 [SMILES: O=C(Nc1nc(-c2ccc(Cl)cn2)co1)c1cnnn1-c1cccnc1] [symbol]
1478271-10-1 [symbol] 2749711-67-7 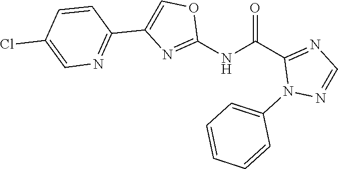 [SMILES: O=C(Nc1nc(-c2ccc(Cl)cn2)co1)c1ncnn1-c1ccccc1] [symbol]
500865-95-2 [symbol] 2749711-67-7 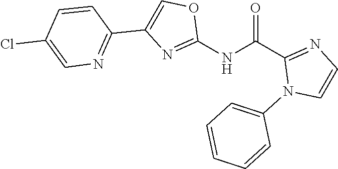 [SMILES: O=C(Nc1nc(-c2ccc(Cl)cn2)co1)c1nccn1-c1ccccc1] [symbol]
516492-57-2 [symbol] 2749711-67-7 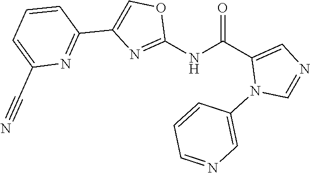 [SMILES: N#Cc1cccc(-c2coc(NC(=O)c3cncn3-c3cccnc3)n2)n1] [symbol]
1197398-67-6 [symbol] 2091017-32-0 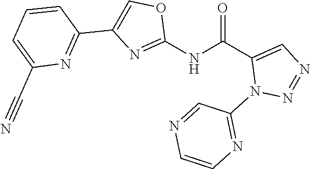 [SMILES: N#Cc1cccc(-c2coc(NC(=O)c3cnnn3-c3cnccn3)n2)n1] [symbol]
2946657-15-2 [symbol] 2091017-32-0 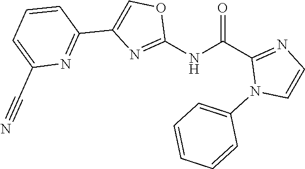 [SMILES: N#Cc1cccc(-c2coc(NC(=O)c3nccn3-c3ccccc3)n2)n1] [symbol]
516492-57-2 [symbol] 2091017-32-0 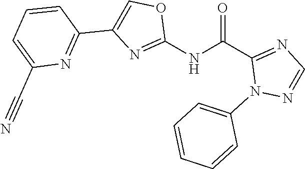 [SMILES: N#Cc1cccc(-c2coc(NC(=O)c3ncnn3-c3ccccc3)n2)n1] [symbol]
500865-95-2 [symbol] 2091017-32-0 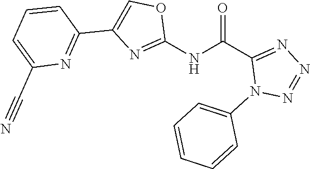 [SMILES: N#Cc1cccc(-c2coc(NC(=O)c3nnnn3-c3ccccc3)n2)n1] [symbol]
214980-86-6 [symbol] 2091017-32-0 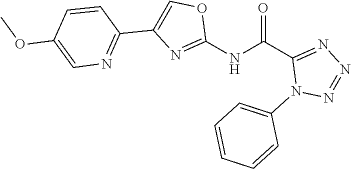 [SMILES: COc1ccc(-c2coc(NC(=O)c3nnnn3-c3ccccc3)n2)nc1] [symbol]
214980-86-6 [symbol] 2749735-50-8 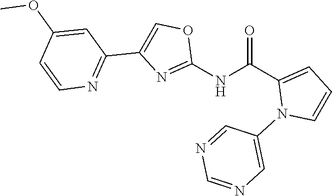 [SMILES: COc1ccnc(-c2coc(NC(=O)c3cccn3-c3cncnc3)n2)c1] [symbol]
1699083-36-7 [symbol] 2740357-86-0 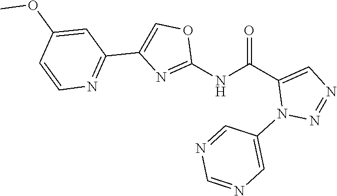 [SMILES: COc1ccnc(-c2coc(NC(=O)c3cnnn3-c3cncnc3)n2)c1] [symbol]
1478093-94-5 [symbol] 2740357-86-0 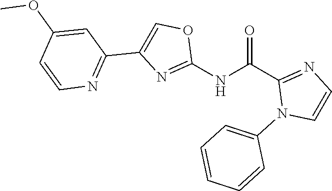 [SMILES: COc1ccnc(-c2coc(NC(=O)c3nccn3-c3ccccc3)n2)c1] [symbol]
516492-57-2 [symbol] 2740357-86-0 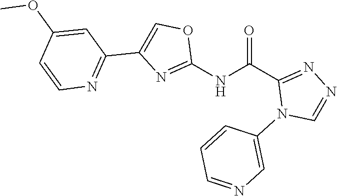 [SMILES: COc1ccnc(-c2coc(NC(=O)c3nncn3-c3cccnc3)n2)c1] [symbol]
2137878-13-6 [symbol] 2740357-86-0 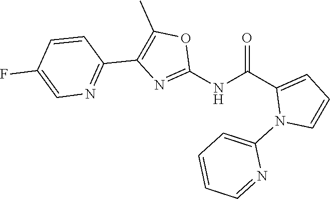 [SMILES: Cc1oc(NC(=O)c2cccn2-c2ccccn2)nc1-c1ccc(F)cn1] [symbol]
896036-05-8 [symbol] 1566302-96-2 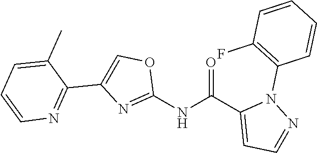 [SMILES: Cc1cccnc1-c1coc(NC(=O)c2ccnn2-c2ccccc2F)n1] [symbol]
described above [symbol] 1501515-08-7 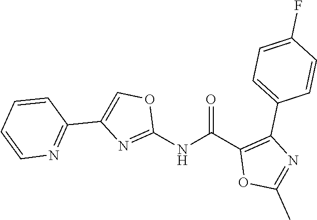 [SMILES: Cc1nc(-c2ccc(F)cc2)c(C(=O)Nc2nc(-c3ccccn3)co2)o1] [symbol]
914287-73-3 [symbol] 1014629-84-5 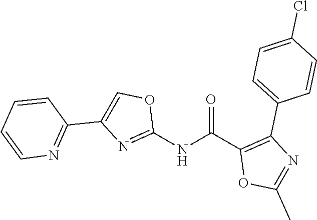 [SMILES: Cc1nc(-c2ccc(Cl)cc2)c(C(=O)Nc2nc(-c3ccccn3)co2)o1] [symbol]
1368745-66-7 [symbol] 1014629-84-5 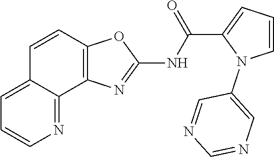 [SMILES: O=C(Nc1nc2c(ccc3cccnc32)o1)c1cccn1-c1cncnc1] [symbol]
1699083-36-7 [symbol] 2519840-67-4 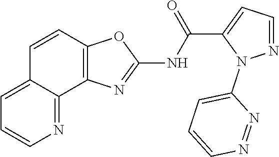 [SMILES: O=C(Nc1nc2c(ccc3cccnc32)o1)c1ccnn1-c1cccnn1] [symbol]
1935682-49-7 [symbol] 2519640-67-4 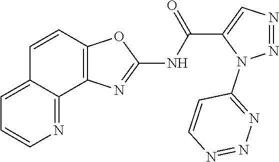 [SMILES: O=C(Nc1nc2c(ccc3cccnc32)o1)c1cnnn1-c1ccnnn1] [symbol]
1482934-49-5 [symbol] 2519640-67-4 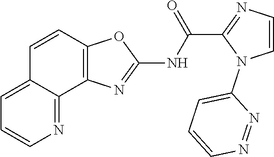 [SMILES: O=C(Nc1nc2c(ccc3cccnc32)o1)c1nccn1-c1cccnn1] [symbol]
1935963-11-3 [symbol] 2519640-67-4 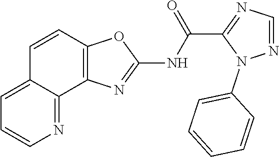 [SMILES: O=C(Nc1nc2c(ccc3cccnc32)o1)c1ncnn1-c1ccccc1] [symbol]
500865-95-2 [symbol] 2519640-67-4 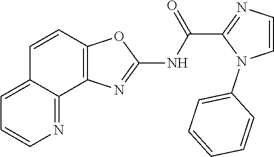 [SMILES: O=C(Nc1nc2c(ccc3cccnc32)o1)c1nccn1-c1ccccc1] [symbol]
516492-57-2 [symbol] 2519640-67-4 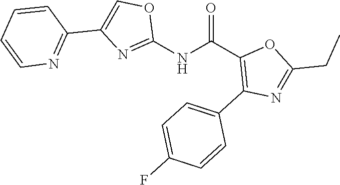 [SMILES: CCc1nc(-c2ccc(F)cc2)c(C(=O)Nc2nc(-c3ccccn3)co2)o1] [symbol]
1489246-42-5 [symbol] 1014629-84-5 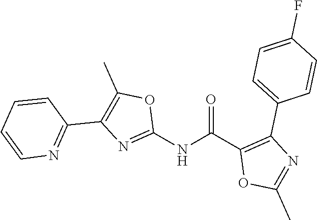 [SMILES: Cc1nc(-c2ccc(F)cc2)c(C(=O)Nc2nc(-c3ccccn3)c(C)o2)o1] [symbol]
914287-73-3 [symbol] 1903307-93-6 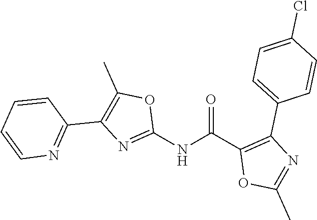 [SMILES: Cc1nc(-c2ccc(Cl)cc2)c(C(=O)Nc2nc(-c3ccccn3)c(C)o2)o1] [symbol]
1368745-66-7 [symbol] 1903307-93-6 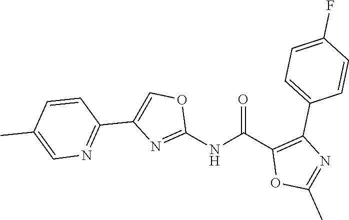 [SMILES: Cc1ccc(-c2coc(NC(=O)c3oc(C)nc3-c3ccc(F)cc3)n2)nc1] [symbol]
914287-73-3 [symbol] 2752153-86-7 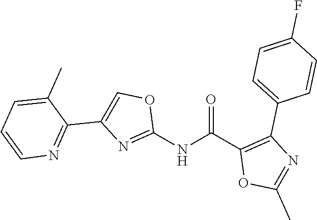 [SMILES: Cc1nc(-c2ccc(F)cc2)c(C(=O)Nc2nc(-c3ncccc3C)co2)o1] [symbol]
described above [symbol] 914287-73-3 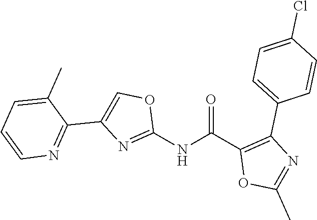 [SMILES: Cc1nc(-c2ccc(Cl)cc2)c(C(=O)Nc2nc(-c3ncccc3C)co2)o1] [symbol]
described above [symbol] 1368745-66-7 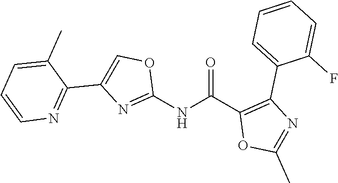 [SMILES: Cc1nc(-c2ccccc2F)c(C(=O)Nc2nc(-c3ncccc3C)co2)o1] [symbol]
described above [symbol] 1368655-22-4 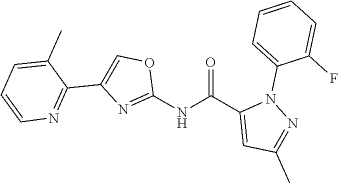 [SMILES: Cc1cc(C(=O)Nc2nc(-c3ncccc3C)co2)n(-c2ccccc2F)n1] [symbol]
described above [symbol] 948293-26-3 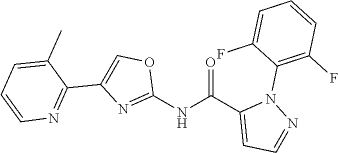 [SMILES: Cc1cccnc1-c1coc(NC(=O)c2ccnn2-c2c(F)cccc2F)n1] [symbol]
described above [symbol] in SSTI patent 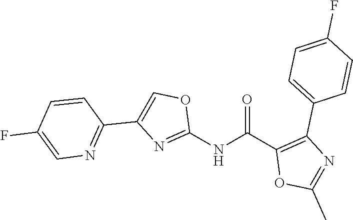 [SMILES: Cc1nc(-c2ccc(F)cc2)c(C(=O)Nc2nc(-c3ccc(F)cn3)co2)o1] [symbol]
914287-73-3 [symbol] 1544907-72-3 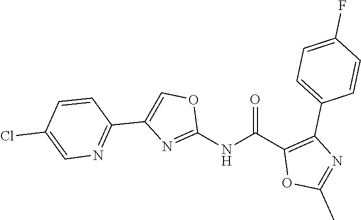 [SMILES: Cc1nc(-c2ccc(F)cc2)c(C(=O)Nc2nc(-c3ccc(Cl)cn3)co2)o1] [symbol]
914287-73-3 [symbol] 2749711-67-7 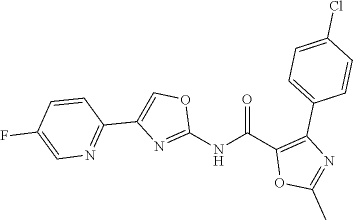 [SMILES: Cc1nc(-c2ccc(Cl)cc2)c(C(=O)Nc2nc(-c3ccc(F)cn3)co2)o1] [symbol]
1368745-66-7 [symbol] 1544907-72-3 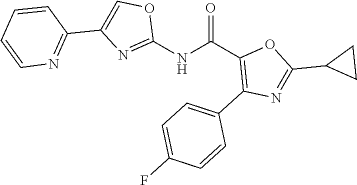 [SMILES: O=C(Nc1nc(-c2ccccn2)co1)c1oc(C2CC2)nc1-c1ccc(F)cc1] [symbol]
described above [symbol] 1014629-84-5 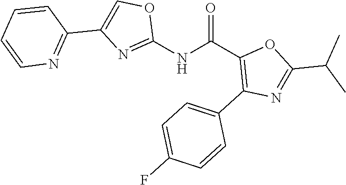 [SMILES: CC(C)c1nc(-c2ccc(F)cc2)c(C(=O)Nc2nc(-c3ccccn3)co2)o1] [symbol]
described above [symbol] 1014629-84-5 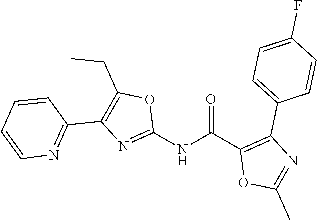 [SMILES: CCc1oc(NC(=O)c2oc(C)nc2-c2ccc(F)cc2)nc1-c1ccccn1] [symbol]
914287-73-3 [symbol] 1904169-47-6 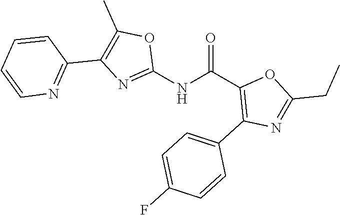 [SMILES: CCc1nc(-c2ccc(F)cc2)c(C(=O)Nc2nc(-c3ccccn3)c(C)o2)o1] [symbol]
1489246-42-5 [symbol] 1903307-93-6 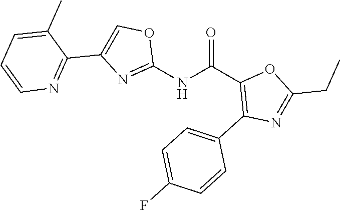 [SMILES: CCc1nc(-c2ccc(F)cc2)c(C(=O)Nc2nc(-c3ncccc3C)co2)o1] [symbol]
described above [symbol] 1489246-42-5 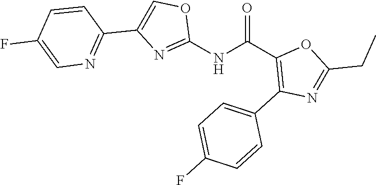 [SMILES: CCc1nc(-c2ccc(F)cc2)c(C(=O)Nc2nc(-c3ccc(F)cn3)co2)o1] [symbol]
1489246-42-5 [symbol] 1544907-72-3 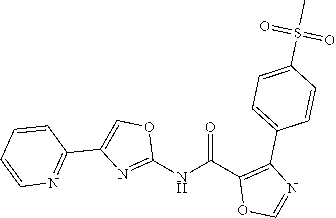 [SMILES: CS(=O)(=O)c1ccc(-c2ncoc2C(=O)Nc2nc(-c3ccccn3)co2)cc1] [symbol]
1519246-15-1 [symbol] 1014629-84-5 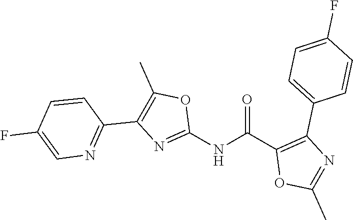 [SMILES: Cc1nc(-c2ccc(F)cc2)c(C(=O)Nc2nc(-c3ccc(F)cn3)c(C)o2)o1] [symbol]
914287-73-3 [symbol] 1566302-96-2 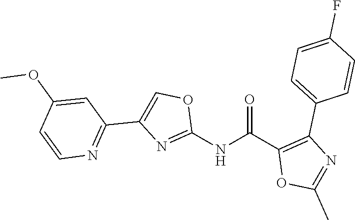 [SMILES: COc1ccnc(-c2coc(NC(=O)c3oc(C)nc3-c3ccc(F)cc3)n2)c1] [symbol]
914267-73-3 [symbol] 2740357-86-0 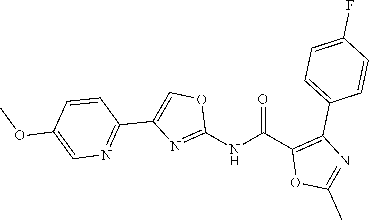 [SMILES: COc1ccc(-c2coc(NC(=O)c3oc(C)nc3-c3ccc(F)cc3)n2)nc1] [symbol]
914287-73-3 [symbol] 2749735-50-8 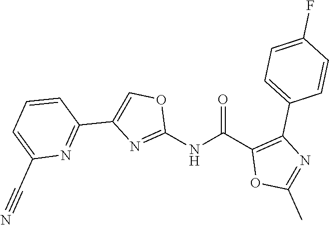 [SMILES: Cc1nc(-c2ccc(F)cc2)c(C(=O)Nc2nc(-c3cccc(C#N)n3)co2)o1] [symbol]
914287-73-3 [symbol] 2091017-32-0 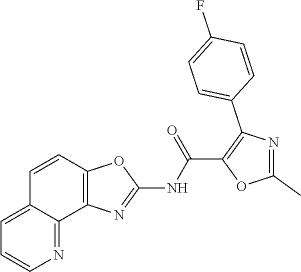 [SMILES: Cc1nc(-c2ccc(F)cc2)c(C(=O)Nc2nc3c(ccc4cccnc43)o2)o1] [symbol]
914287-73-3 [symbol] 2519640-67-4 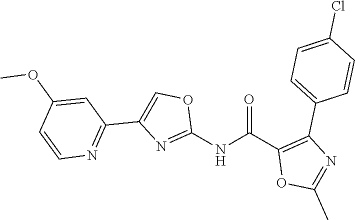 [SMILES: COc1ccnc(-c2coc(NC(=O)c3oc(C)nc3-c3ccc(Cl)cc3)n2)c1] [symbol]
1368745-66-7 [symbol] 2740357-66-0 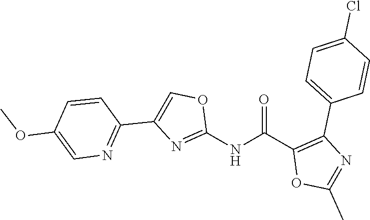 [SMILES: COc1ccc(-c2coc(NC(=O)c3oc(C)nc3-c3ccc(Cl)cc3)n2)nc1] [symbol]
1368745-66-7 [symbol] 2749735-50-8 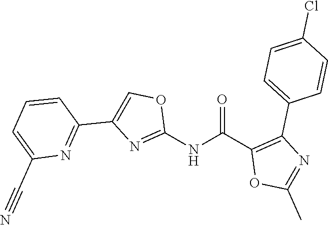 [SMILES: Cc1nc(-c2ccc(Cl)cc2)c(C(=O)Nc2nc(-c3cccc(C#N)n3)co2)o1] [symbol]
1368745-66-7 [symbol] 2091017-32-0 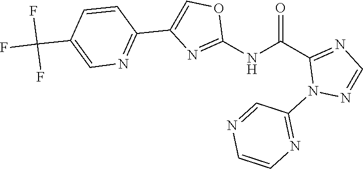 [SMILES: O=C(Nc1nc(-c2ccc(C(F)(F)F)cn2)co1)c1ncnn1-c1cnccn1] [symbol]
2946657-01-6 [symbol] 2748631-89-0 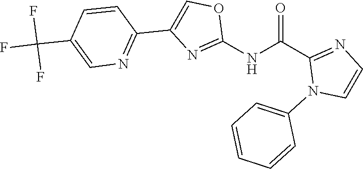 [SMILES: O=C(Nc1nc(-c2ccc(C(F)(F)F)cn2)co1)c1nccn1-c1ccccc1] [symbol]
516492-57-2 [symbol] 2748631-89-0 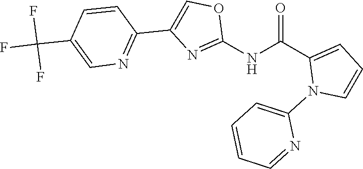 [SMILES: O=C(Nc1nc(-c2ccc(C(F)(F)F)cn2)co1)c1cccn1-c1ccccn1] [symbol]
896036-05-8 [symbol] 2748631-89-0 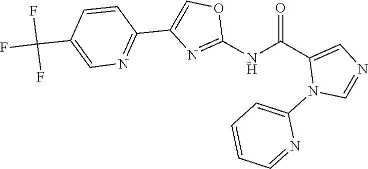 [SMILES: O=C(Nc1nc(-c2ccc(C(F)(F)F)cn2)co1)c1cncn1-c1ccccn1] [symbol]
1779126-15-2 [symbol] 2748631-89-0 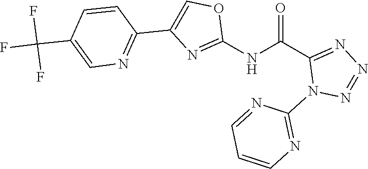 [SMILES: O=C(Nc1nc(-c2ccc(C(F)(F)F)cn2)co1)c1nnnn1-c1ncccn1] [symbol]
2946657-11-8 [symbol] 2748631-89-0 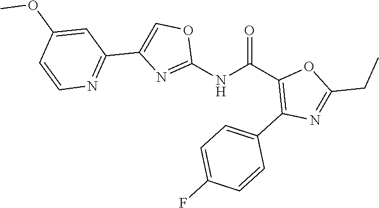 [SMILES: CCc1nc(-c2ccc(F)cc2)c(C(=O)Nc2nc(-c3cc(OC)ccn3)co2)o1] [symbol]
1489246-42-5 [symbol] 2740357-86-0 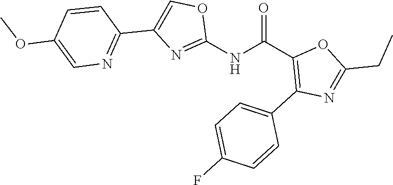 [SMILES: CCc1nc(-c2ccc(F)cc2)c(C(=O)Nc2nc(-c3ccc(OC)cn3)co2)o1] [symbol]
1489246-42-5 [symbol] 2749735-50-8 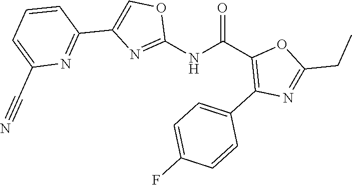 [SMILES: CCc1nc(-c2ccc(F)cc2)c(C(=O)Nc2nc(-c3cccc(C#N)n3)co2)o1] [symbol]
1489246-42-5 [symbol] 2091017-32-0 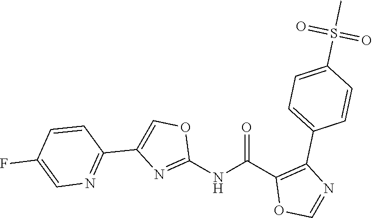 [SMILES: CS(=O)(=O)c1ccc(-c2ncoc2C(=O)Nc2nc(-c3ccc(F)cn3)co2)cc1] [symbol]
1519246-15-1 [symbol] 1544907-72-3 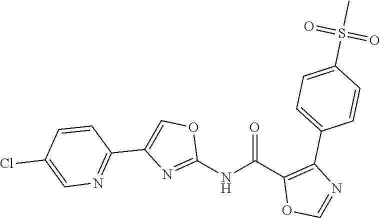 [SMILES: CS(=O)(=O)c1ccc(-c2ncoc2C(=O)Nc2nc(-c3ccc(Cl)cn3)co2)cc1] [symbol]
1519246-15-1 [symbol] 2749711-67-7 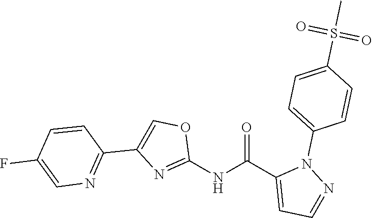 [SMILES: CS(=O)(=O)c1ccc(-n2nccc2C(=O)Nc2nc(-c3ccc(F)cn3)co2)cc1] [symbol]
681454-61-5 [symbol] 1544907-72-3 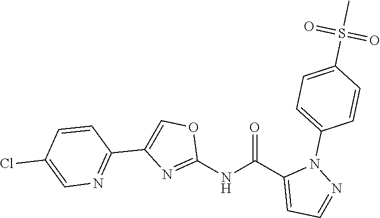 [SMILES: CS(=O)(=O)c1ccc(-n2nccc2C(=O)Nc2nc(-c3ccc(Cl)cn3)co2)cc1] [symbol]
681454-61-5 [symbol] 2749711-67-7 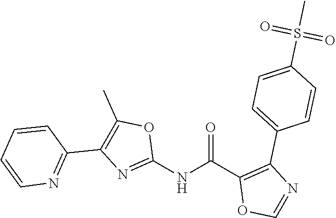 [SMILES: Cc1oc(NC(=O)c2ocnc2-c2ccc(S(C)(=O)=O)cc2)nc1-c1ccccn1] [symbol]
1519246-15-1 [symbol] 1903307-93-8 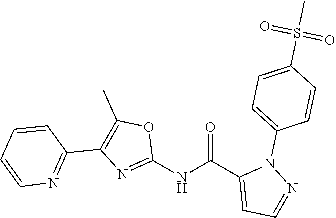 [SMILES: Cc1oc(NC(=O)c2ccnn2-c2ccc(S(C)(=O)=O)cc2)nc1-c1ccccn1] [symbol]
681454-61-5 [symbol] 1903307-93-6 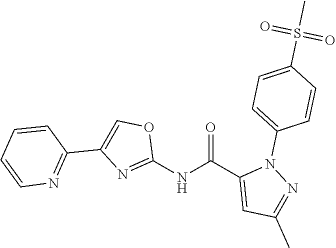 [SMILES: Cc1cc(C(=O)Nc2nc(-c3ccccn3)co2)n(-c2ccc(S(C)(=O)=O)cc2)n1] [symbol]
1020237-76-6 [symbol] 1014629-84-5 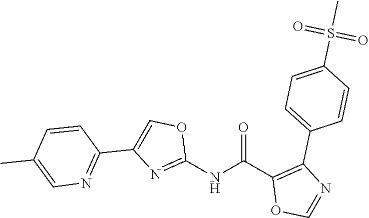 [SMILES: Cc1ccc(-c2coc(NC(=O)c3ocnc3-c3ccc(S(C)(=O)=O)cc3)n2)nc1] [symbol]
1519246-15-1 [symbol] 2752153-86-7 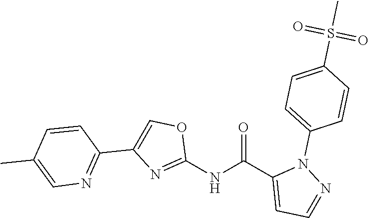 [SMILES: Cc1ccc(-c2coc(NC(=O)c3ccnn3-c3ccc(S(C)(=O)=O)cc3)n2)nc1] [symbol]
681454-61-5 [symbol] 2752153-86-7 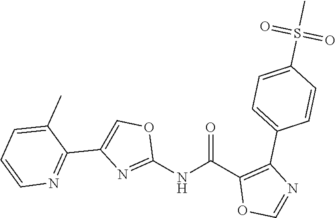 [SMILES: Cc1cccnc1-c1coc(NC(=O)c2ocnc2-c2ccc(S(C)(=O)=O)cc2)n1] [symbol]
described above [symbol] 1519246-15-1 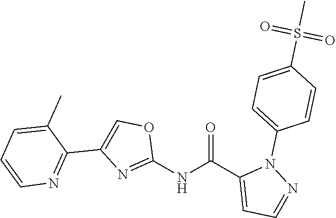 [SMILES: Cc1cccnc1-c1coc(NC(=O)c2ccnn2-c2ccc(S(C)(=O)=O)cc2)n1] [symbol]
described above [symbol] 681454-61-5 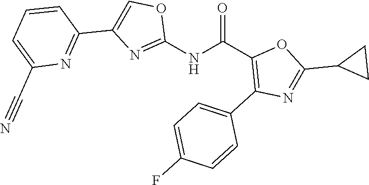 [SMILES: N#Cc1cccc(-c2coc(NC(=O)c3oc(C4CC4)nc3-c3ccc(F)cc3)n2)n1] [symbol]
described above [symbol] 2091017-32-0 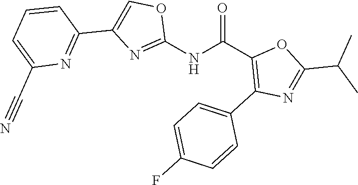 [SMILES: CC(C)c1nc(-c2ccc(F)cc2)c(C(=O)Nc2nc(-c3cccc(C#N)n3)co2)o1] [symbol]
described above [symbol] 2091017-32-0 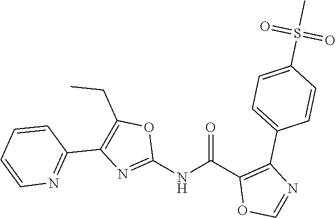 [SMILES: CCc1oc(NC(=O)c2ocnc2-c2ccc(S(C)(=O)=O)cc2)nc1-c1ccccn1] [symbol]
1519246-15-1 [symbol] 1904169-47-6 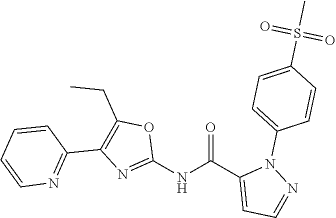 [SMILES: CCc1oc(NC(=O)c2ccnn2-c2ccc(S(C)(=O)=O)cc2)nc1-c1ccccn1] [symbol]
681454-61-5 [symbol] 1904169-47-6 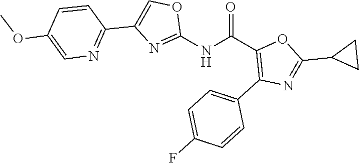 [SMILES: COc1ccc(-c2coc(NC(=O)c3oc(C4CC4)nc3-c3ccc(F)cc3)n2)nc1] [symbol]
described above [symbol] 2749735-50-8 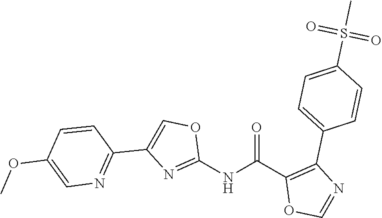 [SMILES: COc1ccc(-c2coc(NC(=O)c3ocnc3-c3ccc(S(C)(=O)=O)cc3)n2)nc1] [symbol]
1519246-15-1 [symbol] 2749735-50-8 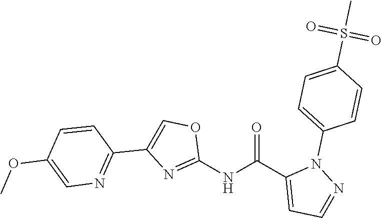 [SMILES: COc1ccc(-c2coc(NC(=O)c3ccnn3-c3ccc(S(C)(=O)=O)cc3)n2)nc1] [symbol]
881454-61-5 [symbol] 2749735-50-8 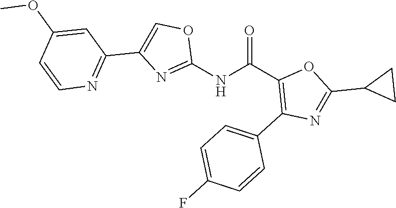 [SMILES: COc1ccnc(-c2coc(NC(=O)c3oc(C4CC4)nc3-c3ccc(F)cc3)n2)c1] [symbol]
described above [symbol] 2740357-86-0 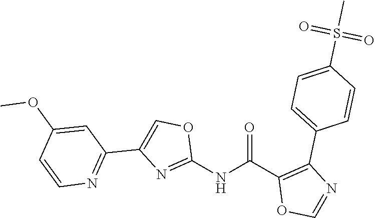 [SMILES: COc1ccnc(-c2coc(NC(=O)c3ocnc3-c3ccc(S(C)(=O)=O)cc3)n2)c1] [symbol]
1519246-15-1 [symbol] 2740357-86-0 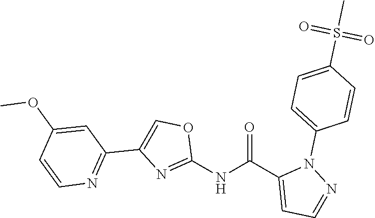 [SMILES: COc1ccnc(-c2coc(NC(=O)c3ccnn3-c3ccc(S(C)(=O)=O)cc3)n2)c1] [symbol]
681454-61-5 [symbol] 2740357-86-0 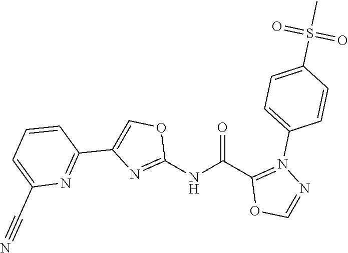 [symbol]
1519246-15-1 [symbol] 2091017-32-0 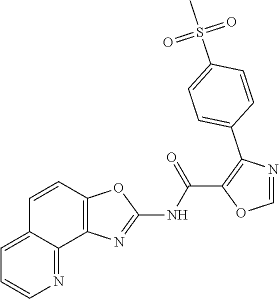 [SMILES: CS(=O)(=O)c1ccc(-c2ncoc2C(=O)Nc2nc3c(ccc4cccnc43)o2)cc1] [symbol]
1519246-15-1 [symbol] 2519640-67-4 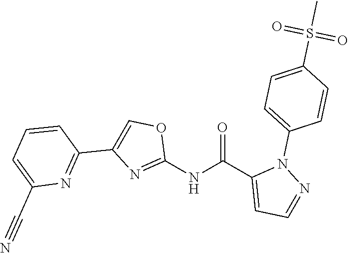 [SMILES: CS(=O)(=O)c1ccc(-n2nccc2C(=O)Nc2nc(-c3cccc(C#N)n3)co2)cc1] [symbol]
681454-61-5 [symbol] 2091017-32-0 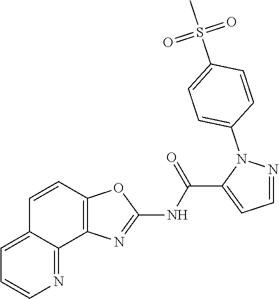 [SMILES: CS(=O)(=O)c1ccc(-n2nccc2C(=O)Nc2nc3c(ccc4cccnc43)o2)cc1] [symbol]
681454-61-5 [symbol] 2519640-67-4 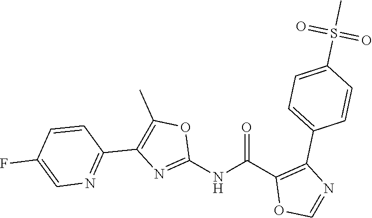 [SMILES: Cc1oc(NC(=O)c2ocnc2-c2ccc(S(C)(=O)=O)cc2)nc1-c1ccc(F)cn1] [symbol]
1519246-15-1 [symbol] 1586302-96-2 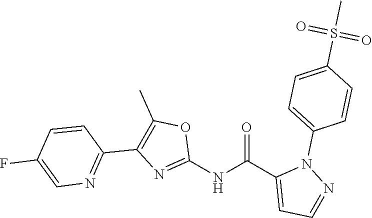 [SMILES: Cc1oc(NC(=O)c2ccnn2-c2ccc(S(C)(=O)=O)cc2)nc1-c1ccc(F)cn1] [symbol]
681454-61-5 [symbol] 1566302-96-2 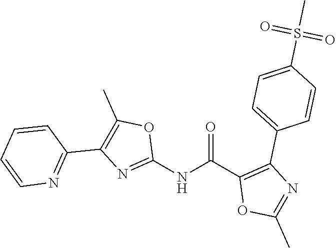 [SMILES: Cc1nc(-c2ccc(S(C)(=O)=O)cc2)c(C(=O)Nc2nc(-c3ccccn3)c(C)o2)o1] [symbol]
described above [symbol] 1903307-93-6 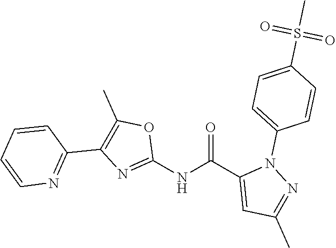 [SMILES: Cc1cc(C(=O)Nc2nc(-c3ccccn3)c(C)o2)n(-c2ccc(S(C)(=O)=O)cc2)n1] [symbol]
1020237-76-6 [symbol] 1903307-93-6 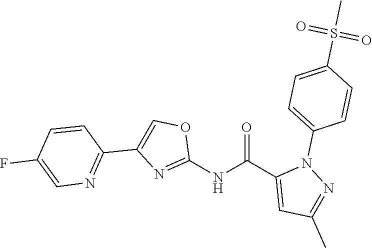 [SMILES: Cc1cc(C(=O)Nc2nc(-c3ccc(F)cn3)co2)n(-c2ccc(S(C)(=O)=O)cc2)n1] [symbol]
1020237-76-6 [symbol] 1544907-72-3 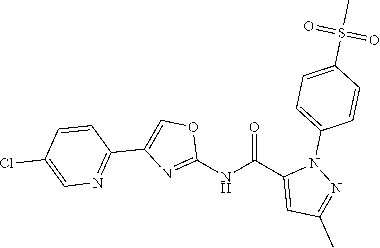 [SMILES: Cc1cc(C(=O)Nc2nc(-c3ccc(Cl)cn3)co2)n(-c2ccc(S(C)(=O)=O)cc2)n1] [symbol]
1020237-76-6 [symbol] 2749711-67-7 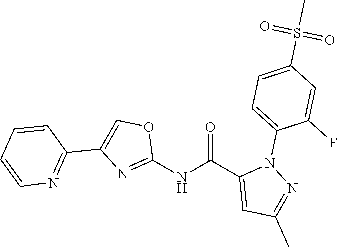 [SMILES: Cc1cc(C(=O)Nc2nc(-c3ccccn3)co2)n(-c2ccc(S(C)(=O)=O)cc2F)n1] [symbol]
described above [symbol] 1014629-84-5 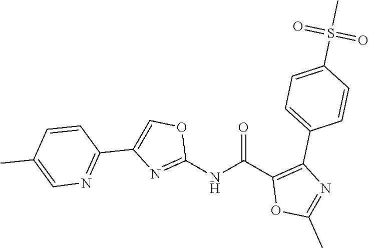 [SMILES: Cc1ccc(-c2coc(NC(=O)c3oc(C)nc3-c3ccc(S(C)(=O)=O)cc3)n2)nc1] [symbol]
described above [symbol] 2752153-86-7 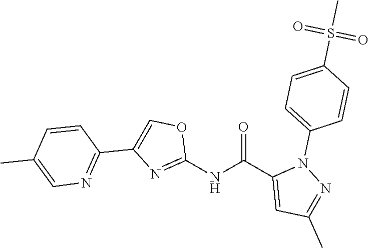 [SMILES: Cc1ccc(-c2coc(NC(=O)c3cc(C)nn3-c3ccc(S(C)(=O)=O)cc3)n2)nc1] [symbol]
1020237-76-6 [symbol] 2752153-86-7 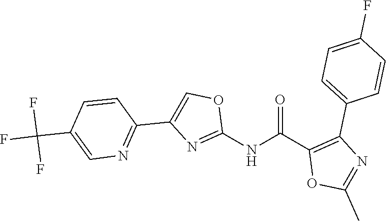 [SMILES: Cc1nc(-c2ccc(F)cc2)c(C(=O)Nc2nc(-c3ccc(C(F)(F)F)cn3)co2)o1] [symbol]
914287-73-3 [symbol] 2748631-89-0 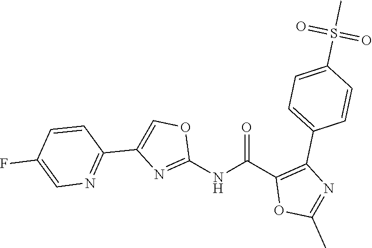 [SMILES: Cc1nc(-c2ccc(S(C)(=O)=O)cc2)c(C(=O)Nc2nc(-c3ccc(F)cn3)co2)o1] [symbol]
described above [symbol] 1544907-72-3 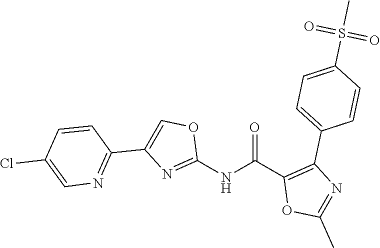 [SMILES: Cc1nc(-c2ccc(S(C)(=O)=O)cc2)c(C(=O)Nc2nc(-c3ccc(Cl)cn3)co2)o1] [symbol]
described above [symbol] 2749711-67-7 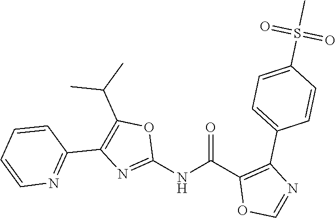 [SMILES: CC(C)c1oc(NC(=O)c2ocnc2-c2ccc(S(C)(=O)=O)cc2)nc1-c1ccccn1] [symbol]
1519246-15-1 [symbol] 1904339-85-0 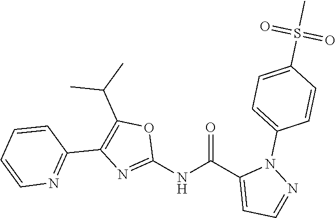 [SMILES: CC(C)c1oc(NC(=O)c2ccnn2-c2ccc(S(C)(=O)=O)cc2)nc1-c1ccccn1] [symbol]
681454-61-5 [symbol] 1904339-85-0 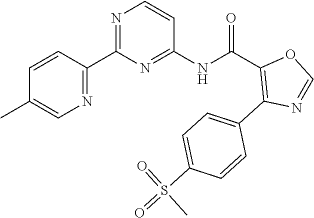 [SMILES: Cc1ccc(-c2nccc(NC(=O)c3ocnc3-c3ccc(S(C)(=O)=O)cc3)n2)nc1] [symbol]
1519258-97-9 [symbol] 1519246-15-1 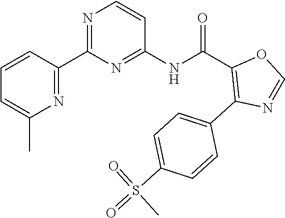 [SMILES: Cc1cccc(-c2nccc(NC(=O)c3ocnc3-c3ccc(S(C)(=O)=O)cc3)n2)n1] [symbol]
1342880-29-8 [symbol] 1519246-15-1 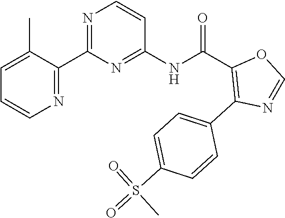 [SMILES: Cc1cccnc1-c1nccc(NC(=O)c2ocnc2-c2ccc(S(C)(=O)=O)cc2)n1] [symbol]
1248303-37-8 [symbol] 1519246-15-1 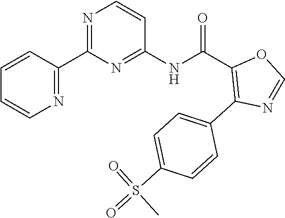 [SMILES: CS(=O)(=O)c1ccc(-c2ncoc2C(=O)Nc2ccnc(-c3ccccn3)n2)cc1] [symbol]
61310-37-0 [symbol] 1519246-15-1 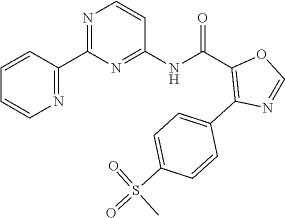 [SMILES: CS(=O)(=O)c1ccc(-c2ncoc2C(=O)Nc2ccnc(-c3ccccn3)n2)cc1] [symbol]
61310-37-0 [symbol] 1519248-15-1 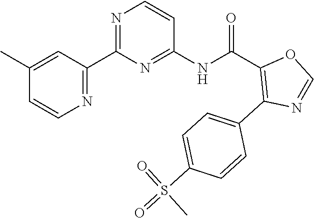 [SMILES: Cc1ccnc(-c2nccc(NC(=O)c3ocnc3-c3ccc(S(C)(=O)=O)cc3)n2)c1] [symbol]
1343999-00-7 [symbol] 1519246-15-1 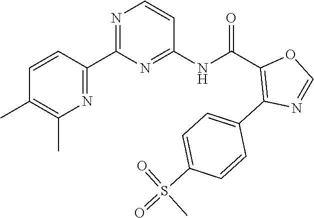 [SMILES: Cc1ccc(-c2nccc(NC(=O)c3ocnc3-c3ccc(S(C)(=O)=O)cc3)n2)nc1C] [symbol]
2008179-82-4 [symbol] 1519246-15-1 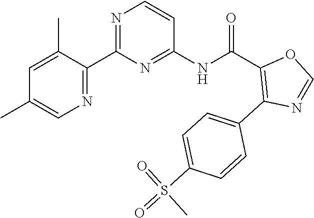 [SMILES: Cc1cnc(-c2nccc(NC(=O)c3ocnc3-c3ccc(S(C)(=O)=O)cc3)n2)c(C)c1] [symbol]
1566121-83-2 [symbol] 1519246-15-1 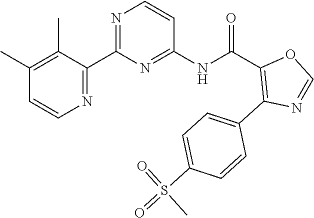 [SMILES: Cc1ccnc(-c2nccc(NC(=O)c3ocnc3-c3ccc(S(C)(=O)=O)cc3)n2)c1C] [symbol]
2281698-08-4 [symbol] 1519246-15-1 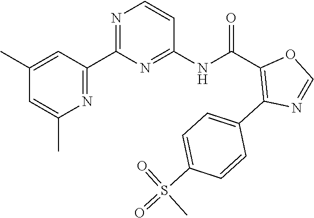 [SMILES: Cc1cc(C)nc(-c2nccc(NC(=O)c3ocnc3-c3ccc(S(C)(=O)=O)cc3)n2)c1] [symbol]
1875153-65-3 [symbol] 1519246-15-1 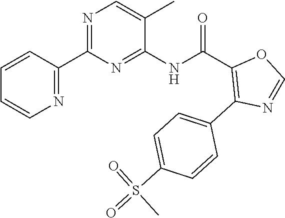 [SMILES: Cc1cnc(-c2ccccn2)nc1NC(=O)c1ocnc1-c1ccc(S(C)(=O)=O)cc1] [symbol]
1702169-08-1 [symbol] 1519245-15-1 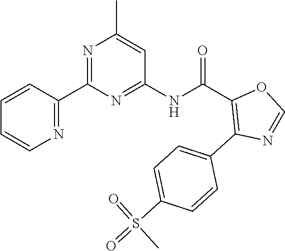 [SMILES: Cc1cc(NC(=O)c2ocnc2-c2ccc(S(C)(=O)=O)cc2)nc(-c2ccccn2)n1] [symbol]
1159822-04-4 [symbol] 1519240-15-1 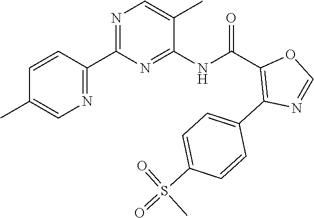 [SMILES: Cc1ccc(-c2ncc(C)c(NC(=O)c3ocnc3-c3ccc(S(C)(=O)=O)cc3)n2)nc1] [symbol]
1999562-38-7 [symbol] 1519246-15-1 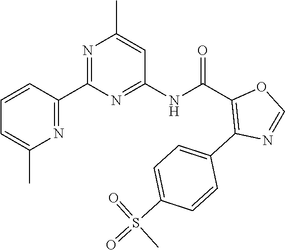 [SMILES: Cc1cccc(-c2nc(C)cc(NC(=O)c3ocnc3-c3ccc(S(C)(=O)=O)cc3)n2)n1] [symbol]
1342083-39-9 [symbol] 1519246-15-1 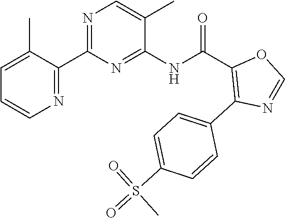 [SMILES: Cc1cnc(-c2ncccc2C)nc1NC(=O)c1ocnc1-c1ccc(S(C)(=O)=O)cc1] [symbol]
1696317-44-8 [symbol] 1519246-15-1 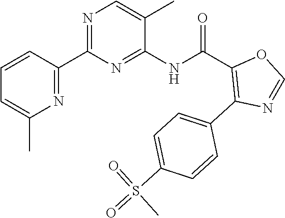 [SMILES: Cc1cccc(-c2ncc(C)c(NC(=O)c3ocnc3-c3ccc(S(C)(=O)=O)cc3)n2)n1] [symbol]
1695654-21-7 [symbol] 1519246-15-1 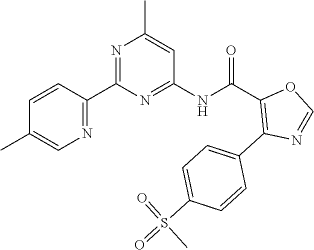 [SMILES: Cc1ccc(-c2nc(C)cc(NC(=O)c3ocnc3-c3ccc(S(C)(=O)=O)cc3)n2)nc1] [symbol]
1501112-02-2 [symbol] 1519246-15-1 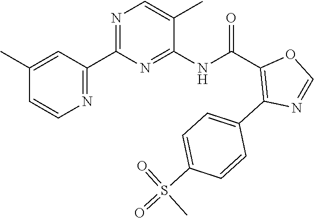 [SMILES: Cc1ccnc(-c2ncc(C)c(NC(=O)c3ocnc3-c3ccc(S(C)(=O)=O)cc3)n2)c1] [symbol]
1697812-36-4 [symbol] 1519246-15-1 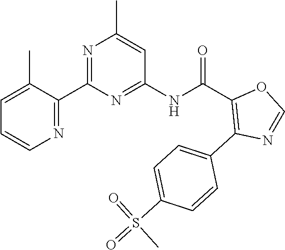 [SMILES: Cc1cc(NC(=O)c2ocnc2-c2ccc(S(C)(=O)=O)cc2)nc(-c2ncccc2C)n1] [symbol]
1247177-40-7 [symbol] 1519248-15-1 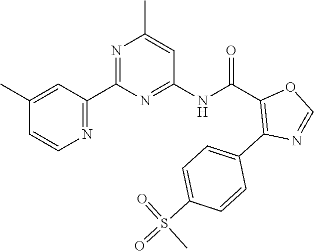 [SMILES: Cc1ccnc(-c2nc(C)cc(NC(=O)c3ocnc3-c3ccc(S(C)(=O)=O)cc3)n2)c1] [symbol]
1275251-87-0 [symbol] 1519248-15-1 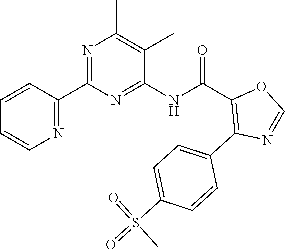 [SMILES: Cc1nc(-c2ccccn2)nc(NC(=O)c2ocnc2-c2ccc(S(C)(=O)=O)cc2)c1C] [symbol]
1248893-25-5 [symbol] 1519246-15-1 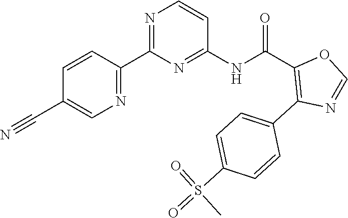 [SMILES: CS(=O)(=O)c1ccc(-c2ncoc2C(=O)Nc2ccnc(-c3ccc(C#N)cn3)n2)cc1] [symbol]
1185315-51-8 [symbol] 1519246-15-1 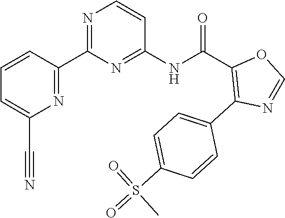 [SMILES: CS(=O)(=O)c1ccc(-c2ncoc2C(=O)Nc2ccnc(-c3cccc(C#N)n3)n2)cc1] [symbol]
1185317-07-0 [symbol] 1519248-15-1 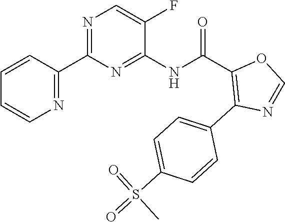 [SMILES: CS(=O)(=O)c1ccc(-c2ncoc2C(=O)Nc2nc(-c3ccccn3)ncc2F)cc1] [symbol]
1863589-09-6 [symbol] 1519248-15-1 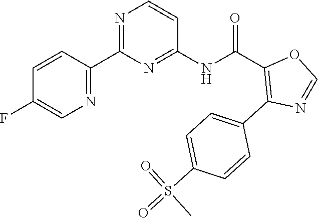 [SMILES: CS(=O)(=O)c1ccc(-c2ncoc2C(=O)Nc2ccnc(-c3ccc(F)cn3)n2)cc1] [symbol]
1503031-04-6 [symbol] 1519245-15-1 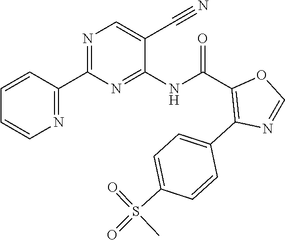 [SMILES: CS(=O)(=O)c1ccc(-c2ncoc2C(=O)Nc2nc(-c3ccccn3)ncc2C#N)cc1] [symbol]
874772-63-1 [symbol] 1519248-15-1 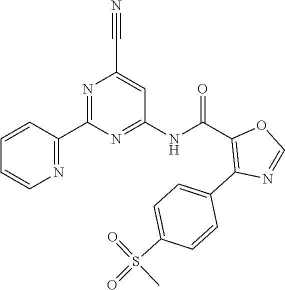 [SMILES: CS(=O)(=O)c1ccc(-c2ncoc2C(=O)Nc2cc(C#N)nc(-c3ccccn3)n2)cc1] [symbol]
1889887-30-2 [symbol] 1519248-15-1 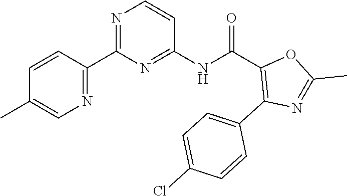 [symbol]
1519258-97-9 [symbol] 1368745-66-7 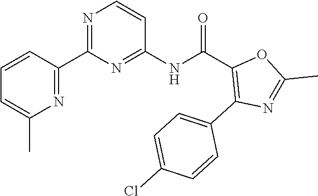 [SMILES: Cc1cccc(-c2nccc(NC(=O)c3oc(C)nc3-c3ccc(Cl)cc3)n2)n1] [symbol]
1342680-29-8 [symbol] 1368745-66-7 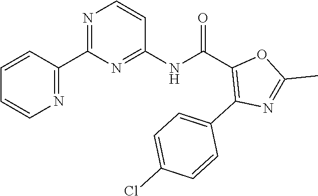 [SMILES: Cc1nc(-c2ccc(Cl)cc2)c(C(=O)Nc2ccnc(-c3ccccn3)n2)o1] [symbol]
61310-37-0 [symbol] 1388745-68-7 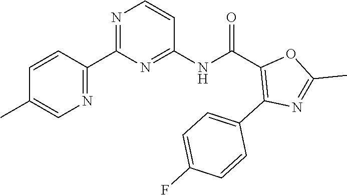 [symbol]
1519258-97-9 [symbol] 914287-73-3 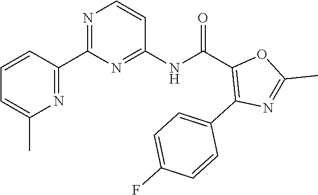 [SMILES: Cc1cccc(-c2nccc(NC(=O)c3oc(C)nc3-c3ccc(F)cc3)n2)n1] [symbol]
1342680-29-8 [symbol] 914287-73-3 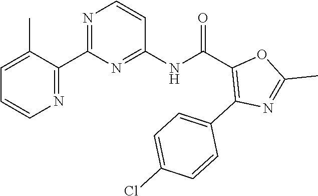 [SMILES: Cc1nc(-c2ccc(Cl)cc2)c(C(=O)Nc2ccnc(-c3ncccc3C)n2)o1] [symbol]
1248303-37-8 [symbol] 1368745-66-7 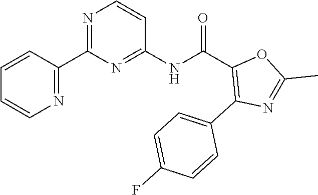 [SMILES: Cc1nc(-c2ccc(F)cc2)c(C(=O)Nc2ccnc(-c3ccccn3)n2)o1] [symbol]
61310-37-0 [symbol] 914287-73-3 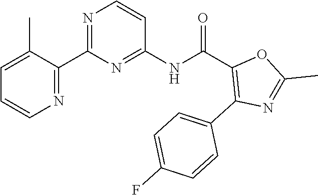 [SMILES: Cc1nc(-c2ccc(F)cc2)c(C(=O)Nc2ccnc(-c3ncccc3C)n2)o1] [symbol]
1248303-37-8 [symbol] 914287-73-3 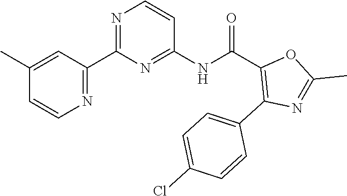 [symbol]
1343999-00-7 [symbol] 1388745-66-7 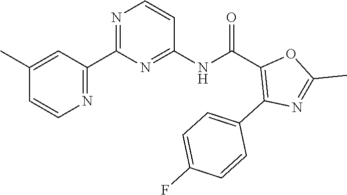 [symbol]
1343999-00-7 [symbol] 914287-73-3 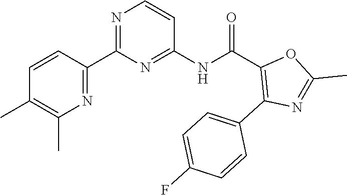 [symbol]
2008179-82-4 [symbol] 914287-73-3 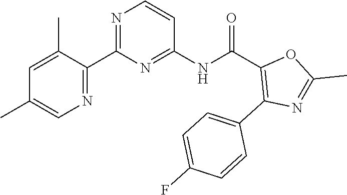 [symbol]
1566121-83-2 [symbol] 914287-73-3 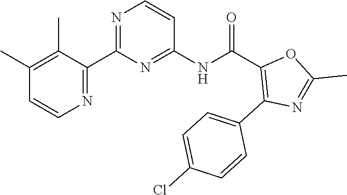 [symbol]
2261698-08-4 [symbol] 1368745-66-7 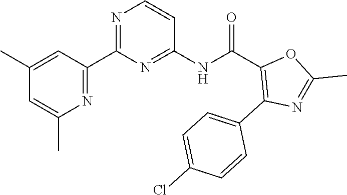 [symbol]
1875153-65-3 [symbol] 1368745-66-7 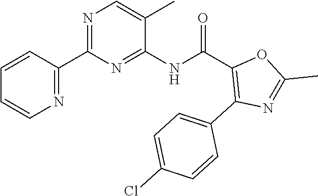 [SMILES: Cc1nc(-c2ccc(Cl)cc2)c(C(=O)Nc2nc(-c3ccccn3)ncc2C)o1] [symbol]
1702169-08-1 [symbol] 1368745-66-7 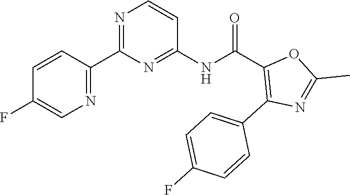 [SMILES: Cc1nc(-c2ccc(F)cc2)c(C(=O)Nc2ccnc(-c3ccc(F)cn3)n2)o1] [symbol]
1503031-04-6 [symbol] 914287-73-3 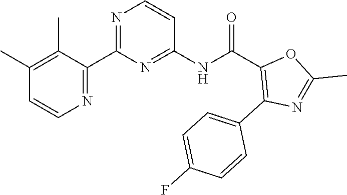 [symbol]
2281698-08-4 [symbol] 914287-73-3 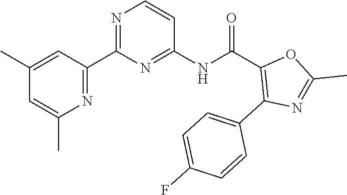 [symbol]
1875153-65-3 [symbol] 914287-73-3 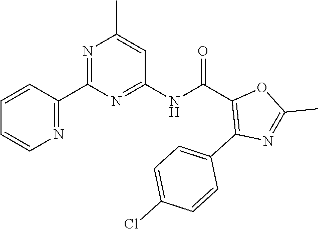 [SMILES: Cc1cc(NC(=O)c2oc(C)nc2-c2ccc(Cl)cc2)nc(-c2ccccn2)n1] [symbol]
1159822-04-4 [symbol] 1368745-66-7 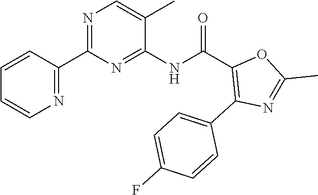 [SMILES: Cc1nc(-c2ccc(F)cc2)c(C(=O)Nc2nc(-c3ccccn3)ncc2C)o1] [symbol]
1702169-08-1 [symbol] 914287-73-3 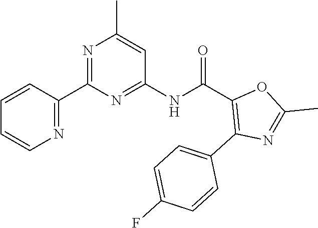 [SMILES: Cc1cc(NC(=O)c2oc(C)nc2-c2ccc(F)cc2)nc(-c2ccccn2)n1] [symbol]
1159822-04-4 [symbol] 914287-73-3 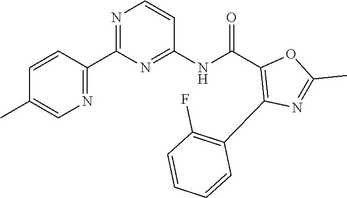 [symbol]
1519258-97-9 [symbol] 1368655-22-4 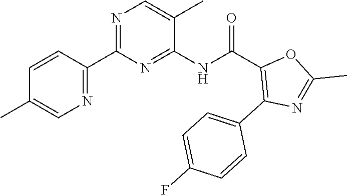 [symbol]
1999562-38-7 [symbol] 914287-73-3 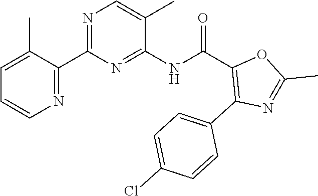 [SMILES: Cc1nc(-c2ccc(Cl)cc2)c(C(=O)Nc2nc(-c3ncccc3C)ncc2C)o1] [symbol]
1698317-44-8 [symbol] 1368745-66-7 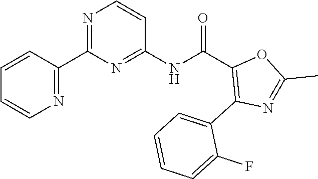 [SMILES: Cc1nc(-c2ccccc2F)c(C(=O)Nc2ccnc(-c3ccccn3)n2)o1] [symbol]
61310-37-0 [symbol] 1368655-22-4 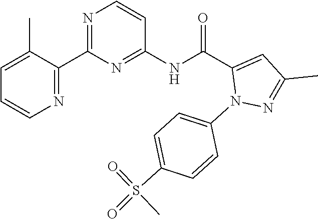 [SMILES: Cc1cc(C(=O)Nc2ccnc(-c3ncccc3C)n2)n(-c2ccc(S(C)(=O)=O)cc2)n1] [symbol]
1248303-37-8 [symbol] 1020237-76-6 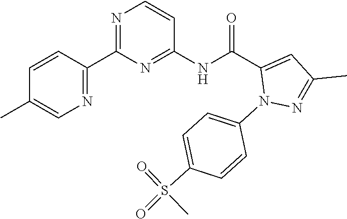 [symbol]
1519258-97-9 [symbol] 1020237-76-6 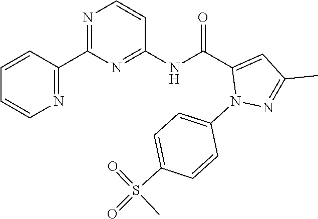 [SMILES: Cc1cc(C(=O)Nc2ccnc(-c3ccccn3)n2)n(-c2ccc(S(C)(=O)=O)cc2)n1] [symbol]
61310-37-0 [symbol] 1020237-78-6 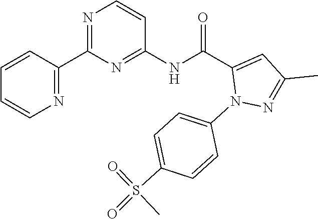 [SMILES: Cc1cc(C(=O)Nc2ccnc(-c3ccccn3)n2)n(-c2ccc(S(C)(=O)=O)cc2)n1] [symbol]
61310-37-0 [symbol] 1020237-76-6 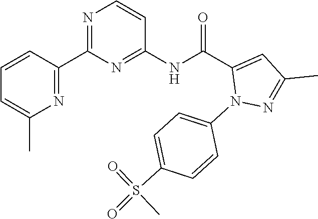 [SMILES: Cc1cccc(-c2nccc(NC(=O)c3cc(C)nn3-c3ccc(S(C)(=O)=O)cc3)n2)n1] [symbol]
1342680-29-8 [symbol] 1020237-76-6 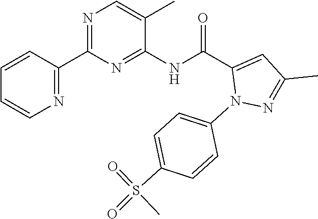 [SMILES: Cc1cc(C(=O)Nc2nc(-c3ccccn3)ncc2C)n(-c2ccc(S(C)(=O)=O)cc2)n1] [symbol]
1702189-08-1 [symbol] 1020237-76-6 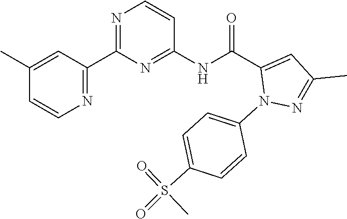 [symbol]
1343999-00-7 [symbol] 1020237-76-6 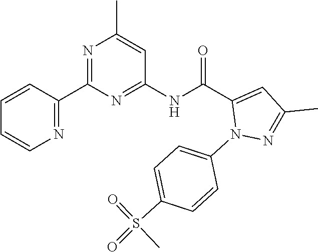 [SMILES: Cc1cc(NC(=O)c2cc(C)nn2-c2ccc(S(C)(=O)=O)cc2)nc(-c2ccccn2)n1] [symbol]
1159822-04-4 [symbol] 1020237-76-6 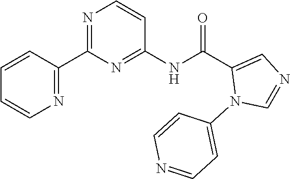 [SMILES: O=C(Nc1ccnc(-c2ccccn2)n1)c1cncn1-c1ccncc1] [symbol]
51310-37-0 [symbol] 1439900-49-8 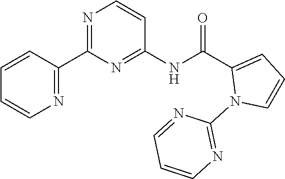 [SMILES: O=C(Nc1ccnc(-c2ccccn2)n1)c1cccn1-c1ncccn1] [symbol]
81310-37-0 [symbol] 896049-60-8 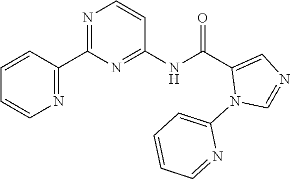 [SMILES: O=C(Nc1ccnc(-c2ccccn2)n1)c1cncn1-c1ccccn1] [symbol]
61310-37-0 [symbol] 1779128-15-2 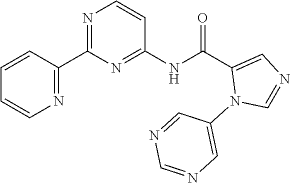 [SMILES: O=C(Nc1ccnc(-c2ccccn2)n1)c1cncn1-c1cncnc1] [symbol]
61310-37-0 [symbol] 1523297-19-9 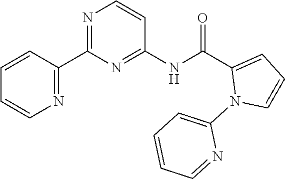 [SMILES: O=C(Nc1ccnc(-c2ccccn2)n1)c1cccn1-c1ccccn1] [symbol]
61310-37-0 [symbol] 896036-05-8 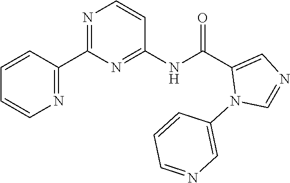 [SMILES: O=C(Nc1ccnc(-c2ccccn2)n1)c1cncn1-c1cccnc1] [symbol]
61310-37-0 [symbol] 1197398-67-6 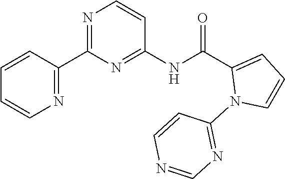 [SMILES: O=C(Nc1ccnc(-c2ccccn2)n1)c1cccn1-c1ccncn1] [symbol]
61310-37-0 [symbol] 1780304-10-0 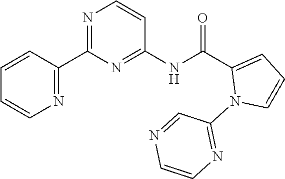 [SMILES: O=C(Nc1ccnc(-c2ccccn2)n1)c1cccn1-c1cnccn1] [symbol]
61310-37-0 [symbol] 896049-56-2 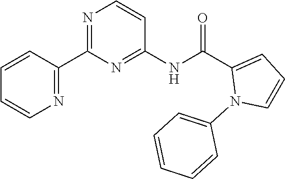 [SMILES: O=C(Nc1ccnc(-c2ccccn2)n1)c1cccn1-c1ccccc1] [symbol]
61310-37-0 [symbol] 78540-03-1 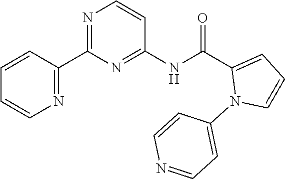 [SMILES: O=C(Nc1ccnc(-c2ccccn2)n1)c1cccn1-c1ccncc1] [symbol]
61310-37-0 [symbol] 1188434-76-5 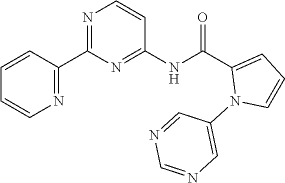 [SMILES: O=C(Nc1ccnc(-c2ccccn2)n1)c1cccn1-c1cncnc1] [symbol]
61310-37-0 [symbol] 1699083-36-7 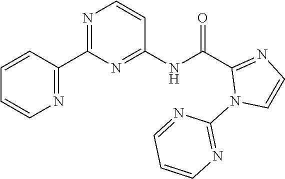 [SMILES: O=C(Nc1ccnc(-c2ccccn2)n1)c1nccn1-c1ncccn1] [symbol]
61310-37-0 [symbol] 1934774-12-5 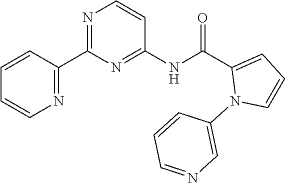 [SMILES: O=C(Nc1ccnc(-c2ccccn2)n1)c1cccn1-c1cccnc1] [symbol]
61310-37-0 [symbol] 896035-98-6 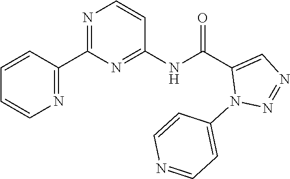 [SMILES: O=C(Nc1ccnc(-c2ccccn2)n1)c1cnnn1-c1ccncc1] [symbol]
61310-37-0 [symbol] 1525417-94-0 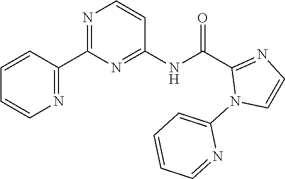 [SMILES: O=C(Nc1ccnc(-c2ccccn2)n1)c1nccn1-c1ccccn1] [symbol]
61310-37-0 [symbol] 1780304-58-6 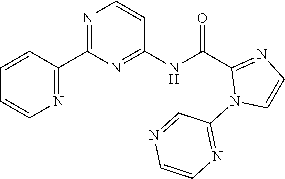 [SMILES: O=C(Nc1ccnc(-c2ccccn2)n1)c1nccn1-c1cnccn1] [symbol]
61310-37-0 [symbol] 1936683-80-5 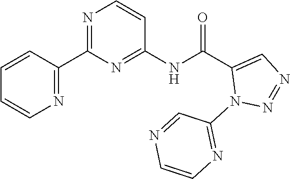 [SMILES: O=C(Nc1ccnc(-c2ccccn2)n1)c1cnnn1-c1cnccn1] [symbol]
61310-37-0 [symbol] 1478093-94-5 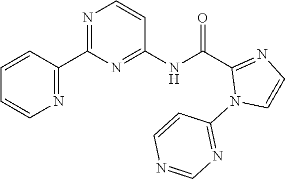 [SMILES: O=C(Nc1ccnc(-c2ccccn2)n1)c1nccn1-c1ccncn1] [symbol]
61310-37-0 [symbol] 1936002-41-3 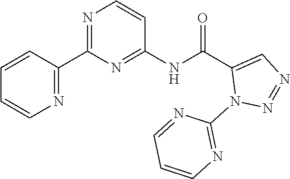 [SMILES: O=C(Nc1ccnc(-c2ccccn2)n1)c1cnnn1-c1ncccn1] [symbol]
61310-37-0 [symbol] 2946657-14-1 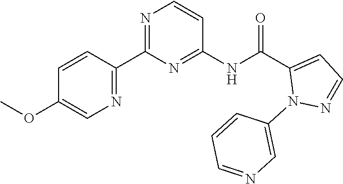 [SMILES: COc1ccc(-c2nccc(NC(=O)c3ccnn3-c3cccnc3)n2)nc1] [symbol]
1851666-45-9 [symbol] 1884400-80-9 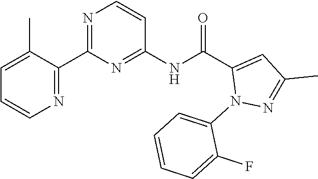 [SMILES: Cc1cc(C(=O)Nc2ccnc(-c3ncccc3C)n2)n(-c2ccccc2F)n1] [symbol]
1248303-37-8 [symbol] 948293-26-3 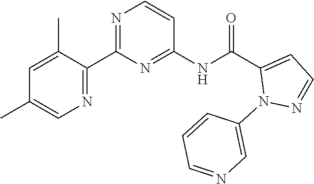 [SMILES: Cc1cnc(-c2nccc(NC(=O)c3ccnn3-c3cccnc3)n2)c(C)c1] [symbol]
1566121-83-2 [symbol] 1884400-80-9 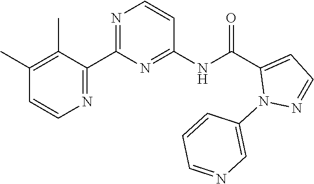 [SMILES: Cc1ccnc(-c2nccc(NC(=O)c3ccnn3-c3cccnc3)n2)c1C] [symbol]
2281698-08-4 [symbol] 1884400-80-9 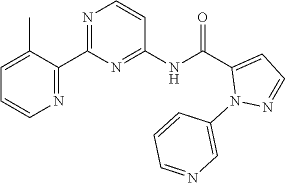 [SMILES: Cc1cccnc1-c1nccc(NC(=O)c2ccnn2-c2cccnc2)n1] [symbol]
1248303-37-8 [symbol] 1884400-80-9 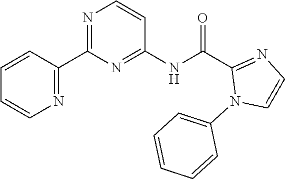 [SMILES: O=C(Nc1ccnc(-c2ccccn2)n1)c1nccn1-c1ccccc1] [symbol]
61310-37-0 [symbol] 516492-57-2 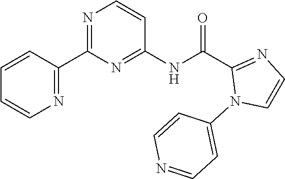 [SMILES: O=C(Nc1ccnc(-c2ccccn2)n1)c1nccn1-c1ccncc1] [symbol]
61310-37-0 [symbol] 1503339-47-6 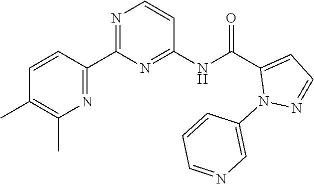 [SMILES: Cc1ccc(-c2nccc(NC(=O)c3ccnn3-c3cccnc3)n2)nc1C] [symbol]
2008179-82-4 [symbol] 1884400-80-9 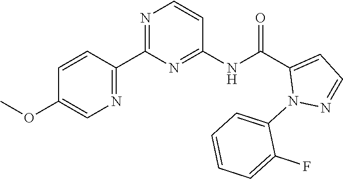 [SMILES: COc1ccc(-c2nccc(NC(=O)c3ccnn3-c3ccccc3F)n2)nc1] [symbol]
1851666-45-9 [symbol] 1501515-08-7 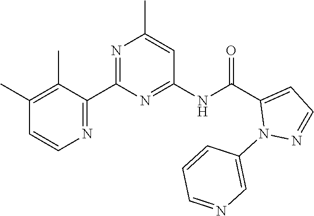 [SMILES: Cc1cc(NC(=O)c2ccnn2-c2cccnc2)nc(-c2nccc(C)c2C)n1] [symbol]
2288346-60-9 [symbol] 1884400-80-9 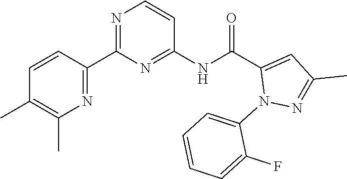 [symbol]
2008179-82-4 [symbol] 048293-26-3 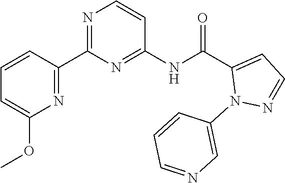 [SMILES: COc1cccc(-c2nccc(NC(=O)c3ccnn3-c3cccnc3)n2)n1] [symbol]
1698491-43-8 [symbol] 1884400-80-9 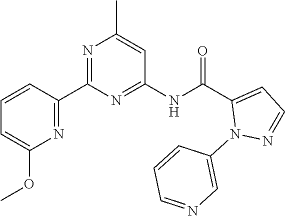 [SMILES: COc1cccc(-c2nc(C)cc(NC(=O)c3ccnn3-c3cccnc3)n2)n1] [symbol]
1694070-76-2 [symbol] 1884400-80-9 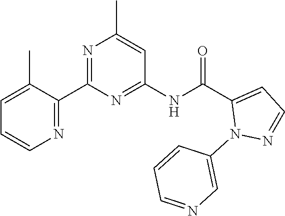 [SMILES: Cc1cc(NC(=O)c2ccnn2-c2cccnc2)nc(-c2ncccc2C)n1] [symbol]
1247177-40-7 [symbol] 1884400-80-9 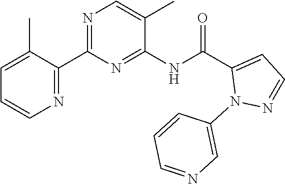 [SMILES: Cc1cnc(-c2ncccc2C)nc1NC(=O)c1ccnn1-c1cccnc1] [symbol]
1696317-44-8 [symbol] 1884400-80-9 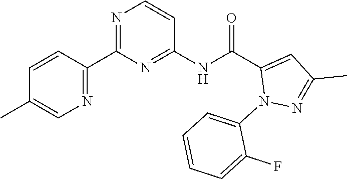 [symbol]
1519258-97-9 [symbol] 948293-26-3 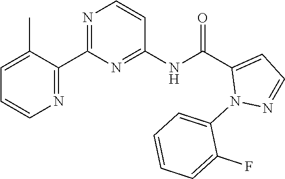 [SMILES: Cc1cccnc1-c1nccc(NC(=O)c2ccnn2-c2ccccc2F)n1] [symbol]
1248303-37-8 [symbol] 1501515-08-7 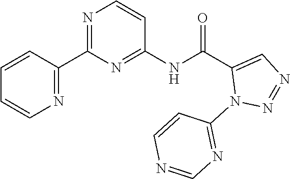 [SMILES: O=C(Nc1ccnc(-c2ccccn2)n1)c1cnnn1-c1ccncn1] [symbol]
61310-37-0 [symbol] 1482934-49-5 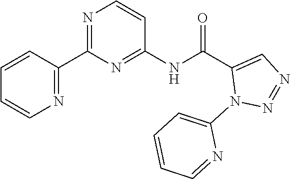 [SMILES: O=C(Nc1ccnc(-c2ccccn2)n1)c1cnnn1-c1ccccn1] [symbol]
61310-37-0 [symbol] 1480445-52-0 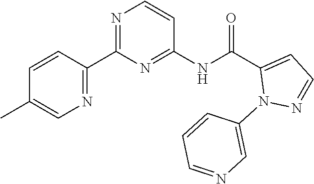 [SMILES: Cc1ccc(-c2nccc(NC(=O)c3ccnn3-c3cccnc3)n2)nc1] [symbol]
1519258-97-9 [symbol] 1884400-80-9 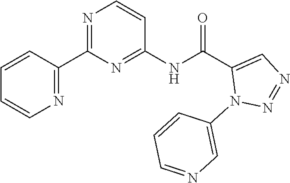 [SMILES: O=C(Nc1ccnc(-c2ccccn2)n1)c1cnnn1-c1cccnc1] [symbol]
61310-37-0 [symbol] 1478271-10-1 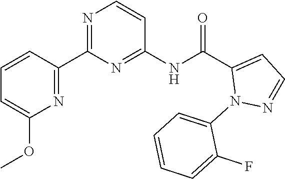 [SMILES: COc1cccc(-c2nccc(NC(=O)c3ccnn3-c3ccccc3F)n2)n1] [symbol]
1698491-43-8 [symbol] 1501515-08-7 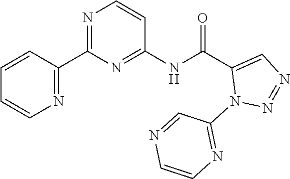 [SMILES: O=C(Nc1ccnc(-c2ccccn2)n1)c1cnnn1-c1cnccn1] [symbol]
61310-37-0 [symbol] 2946657-15-2 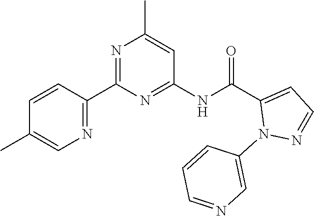 [SMILES: Cc1ccc(-c2nc(C)cc(NC(=O)c3ccnn3-c3cccnc3)n2)nc1] [symbol]
1501112-02-2 [symbol] 1884400-80-9 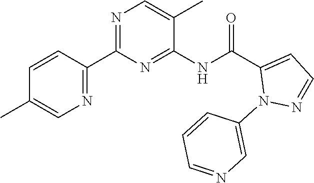 [SMILES: Cc1ccc(-c2ncc(C)c(NC(=O)c3ccnn3-c3cccnc3)n2)nc1] [symbol]
1999562-38-7 [symbol] 1884400-80-9 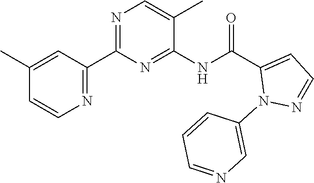 [SMILES: Cc1ccnc(-c2ncc(C)c(NC(=O)c3ccnn3-c3cccnc3)n2)c1] [symbol]
1697812-36-4 [symbol] 1884400-80-9 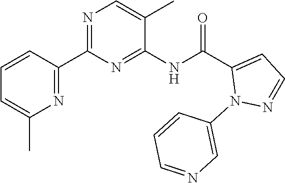 [SMILES: Cc1cccc(-c2ncc(C)c(NC(=O)c3ccnn3-c3cccnc3)n2)n1] [symbol]
1695654-21-7 [symbol] 1884400-80-9 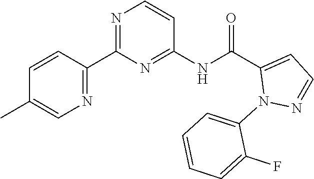 [SMILES: Cc1ccc(-c2nccc(NC(=O)c3ccnn3-c3ccccc3F)n2)nc1] [symbol]
1519258-97-9 [symbol] 1501515-08-7 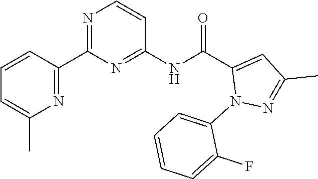 [SMILES: Cc1cccc(-c2nccc(NC(=O)c3cc(C)nn3-c3ccccc3F)n2)n1] [symbol]
1342680-29-8 [symbol] 948293-26-3 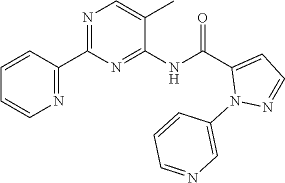 [SMILES: Cc1cnc(-c2ccccn2)nc1NC(=O)c1ccnn1-c1cccnc1] [symbol]
1702169-08-1 [symbol] 1884400-80-9 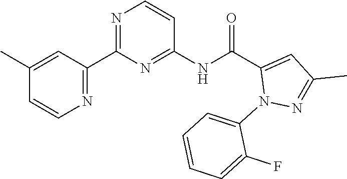 [symbol]
1343999-00-7 [symbol] 948293-26-3 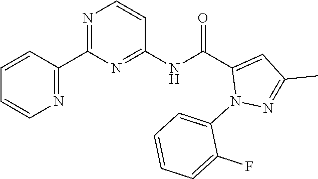 [SMILES: Cc1cc(C(=O)Nc2ccnc(-c3ccccn3)n2)n(-c2ccccc2F)n1] [symbol]
61310-37-0 [symbol] 948293-26-3 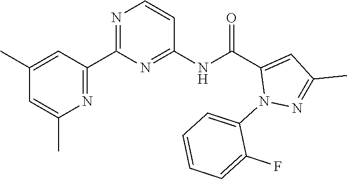 [symbol]
1875153-65-3 [symbol] 948293-28-3 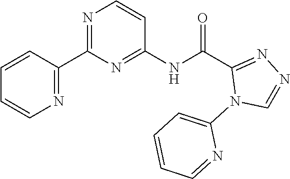 [SMILES: O=C(Nc1ccnc(-c2ccccn2)n1)c1nncn1-c1ccccn1] [symbol]
61310-37-0 [symbol] 2167196-30-5 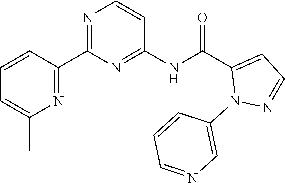 [SMILES: Cc1cccc(-c2nccc(NC(=O)c3ccnn3-c3cccnc3)n2)n1] [symbol]
1342680-29-8 [symbol] 1884400-80-9 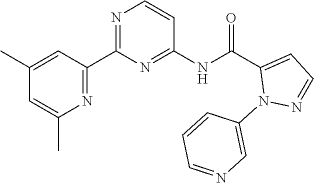 [SMILES: Cc1cc(C)nc(-c2nccc(NC(=O)c3ccnn3-c3cccnc3)n2)c1] [symbol]
1875153-65-3 [symbol] 1884400-80-9 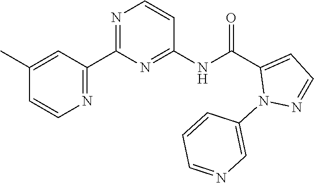 [SMILES: Cc1ccnc(-c2nccc(NC(=O)c3ccnn3-c3cccnc3)n2)c1] [symbol]
1343999-00-7 [symbol] 1884400-80-9 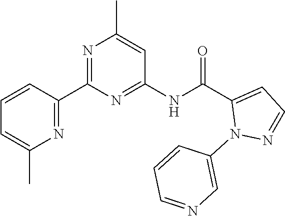 [SMILES: Cc1cccc(-c2nc(C)cc(NC(=O)c3ccnn3-c3cccnc3)n2)n1] [symbol]
1342083-39-9 [symbol] 1884400-80-9 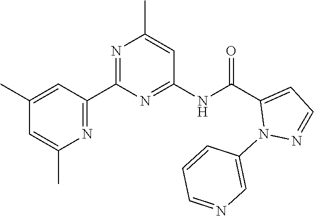 [SMILES: Cc1cc(C)nc(-c2nc(C)cc(NC(=O)c3ccnn3-c3cccnc3)n2)c1] [symbol]
1876867-81-0 [symbol] 1884400-80-9 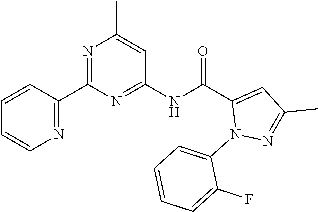 [SMILES: Cc1cc(NC(=O)c2cc(C)nn2-c2ccccc2F)nc(-c2ccccn2)n1] [symbol]
1159822-04-4 [symbol] 948293-26-3 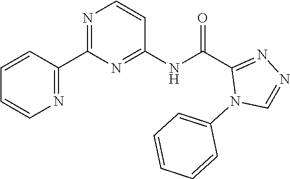 [SMILES: O=C(Nc1ccnc(-c2ccccn2)n1)c1nncn1-c1ccccc1] [symbol]
61310-37-0 [symbol] 1526547-27-2 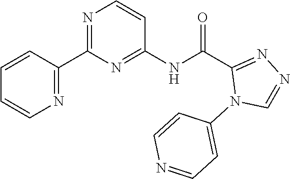 [SMILES: O=C(Nc1ccnc(-c2ccccn2)n1)c1nncn1-c1ccncc1] [symbol]
61310-37-0 [symbol] 2168395-35-3 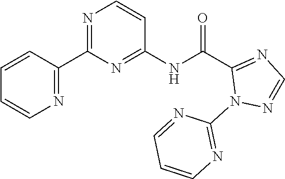 [SMILES: O=C(Nc1ccnc(-c2ccccn2)n1)c1ncnn1-c1ncccn1] [symbol]
61310-37-0 [symbol] 2741044-70-0 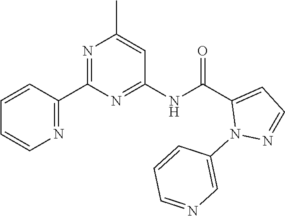 [SMILES: Cc1cc(NC(=O)c2ccnn2-c2cccnc2)nc(-c2ccccn2)n1] [symbol]
1159822-04-4 [symbol] 1884400-80-9 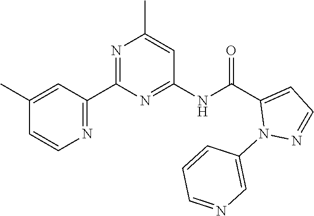 [SMILES: Cc1ccnc(-c2nc(C)cc(NC(=O)c3ccnn3-c3cccnc3)n2)c1] [symbol]
1275251-87-0 [symbol] 1884400-80-9 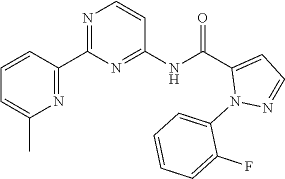 [SMILES: Cc1cccc(-c2nccc(NC(=O)c3ccnn3-c3ccccc3F)n2)n1] [symbol]
1342660-29-8 [symbol] 1501515-08-7 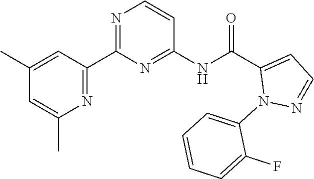 [SMILES: Cc1cc(C)nc(-c2nccc(NC(=O)c3ccnn3-c3ccccc3F)n2)c1] [symbol]
1875153-65-3 [symbol] 1501515-08-7 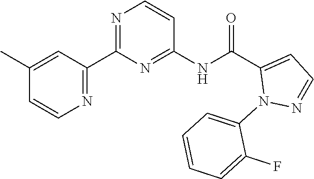 [SMILES: Cc1ccnc(-c2nccc(NC(=O)c3ccnn3-c3ccccc3F)n2)c1] [symbol]
1343999-00-7 [symbol] 1501515-08-7 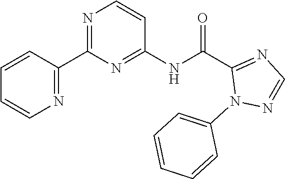 [SMILES: O=C(Nc1ccnc(-c2ccccn2)n1)c1ncnn1-c1ccccc1] [symbol]
61310-37-0 [symbol] 500885-95-2 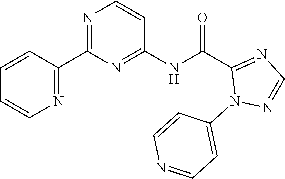 [SMILES: O=C(Nc1ccnc(-c2ccccn2)n1)c1ncnn1-c1ccncc1] [symbol]
61310-37-0 [symbol] 1934935-63-3 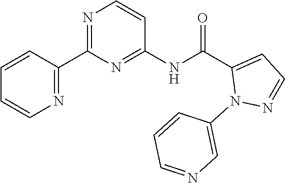 [SMILES: O=C(Nc1ccnc(-c2ccccn2)n1)c1ccnn1-c1cccnc1] [symbol]
61310-37-0 [symbol] 1884400-80-9 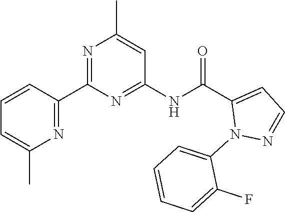 [SMILES: Cc1cccc(-c2nc(C)cc(NC(=O)c3ccnn3-c3ccccc3F)n2)n1] [symbol]
1342083-39-9 [symbol] 1501515-08-7 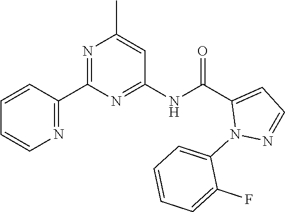 [SMILES: Cc1cc(NC(=O)c2ccnn2-c2ccccc2F)nc(-c2ccccn2)n1] [symbol]
1159822-04-4 [symbol] 1501515-08-7 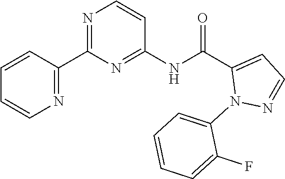 [SMILES: O=C(Nc1ccnc(-c2ccccn2)n1)c1ccnn1-c1ccccc1F] [symbol]
61310-37-0 [symbol] 1501515-08-7 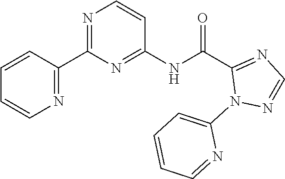 [SMILES: O=C(Nc1ccnc(-c2ccccn2)n1)c1ncnn1-c1ccccn1] [symbol]
61310-37-0 [symbol] 2169404-66-2 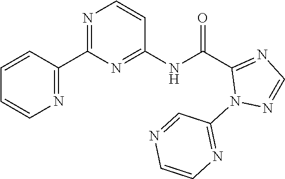 [SMILES: O=C(Nc1ccnc(-c2ccccn2)n1)c1ncnn1-c1cnccn1] [symbol]
61310-37-0 [symbol] 2946657-01-6 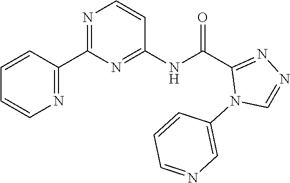 [SMILES: O=C(Nc1ccnc(-c2ccccn2)n1)c1nncn1-c1cccnc1] [symbol]
61310-37-0 [symbol] 2137878-13-6 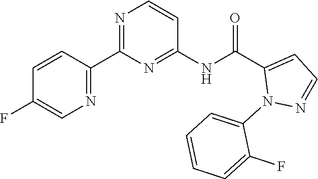 [SMILES: O=C(Nc1ccnc(-c2ccc(F)cn2)n1)c1ccnn1-c1ccccc1F] [symbol]
1503031-04-6 [symbol] 1501515-06-7 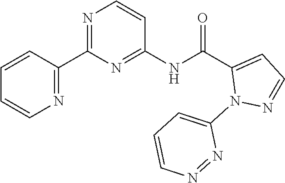 [SMILES: O=C(Nc1ccnc(-c2ccccn2)n1)c1ccnn1-c1cccnn1] [symbol]
61310-37-0 [symbol] 1935682-49-7 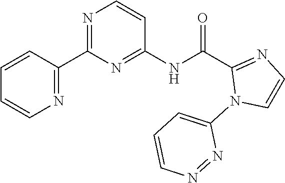 [SMILES: O=C(Nc1ccnc(-c2ccccn2)n1)c1nccn1-c1cccnn1] [symbol]
61310-37-0 [symbol] 1935963-11-3 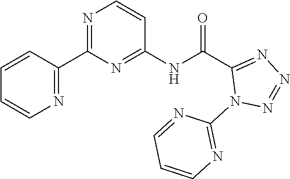 [SMILES: O=C(Nc1ccnc(-c2ccccn2)n1)c1nnnn1-c1ncccn1] [symbol]
61310-37-0 [symbol] 2946657-11-8 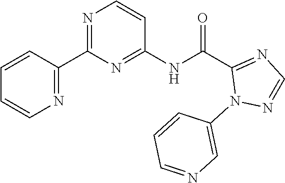 [SMILES: O=C(Nc1ccnc(-c2ccccn2)n1)c1ncnn1-c1cccnc1] [symbol]
61310-37-0 [symbol] 2167545-89-1 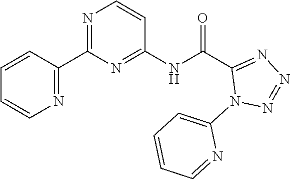 [SMILES: O=C(Nc1ccnc(-c2ccccn2)n1)c1nnnn1-c1ccccn1] [symbol]
61310-37-0 [symbol] 1903206-04-1 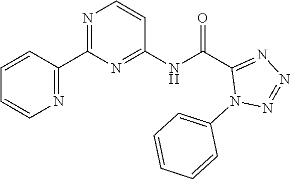 [SMILES: O=C(Nc1ccnc(-c2ccccn2)n1)c1nnnn1-c1ccccc1] [symbol]
51310-37-0 [symbol] 214980-86-6 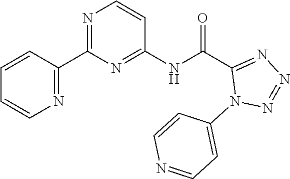 [SMILES: O=C(Nc1ccnc(-c2ccccn2)n1)c1nnnn1-c1ccncc1] [symbol]
61310-37-0 [symbol] 1903870-12-1 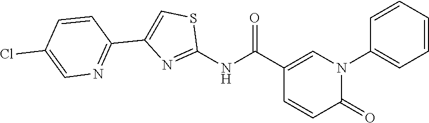 [SMILES: O=C(Nc1nc(-c2ccc(Cl)cn2)cs1)c1ccc(=O)n(-c2ccccc2)c1] [symbol]
1185315-17-6 [symbol] 1189982-99-7 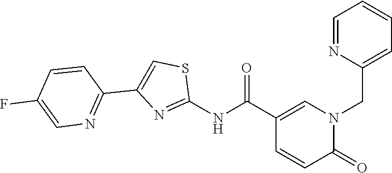 [SMILES: O=C(Nc1nc(-c2ccc(F)cn2)cs1)c1ccc(=O)n(Cc2ccccn2)c1] [symbol]
1551651-30-9 [symbol] 933728-85-9 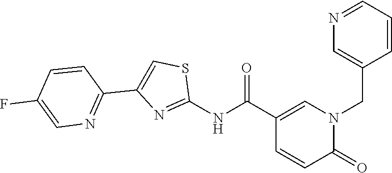 [SMILES: O=C(Nc1nc(-c2ccc(F)cn2)cs1)c1ccc(=O)n(Cc2cccnc2)c1] [symbol]
1551651-30-9 [symbol] 933754-90-6 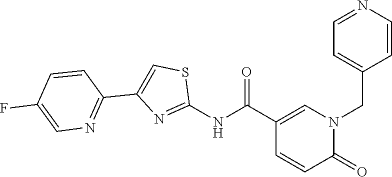 [SMILES: O=C(Nc1nc(-c2ccc(F)cn2)cs1)c1ccc(=O)n(Cc2ccncc2)c1] [symbol]
1551551-30-9 [symbol] 858980-96-8 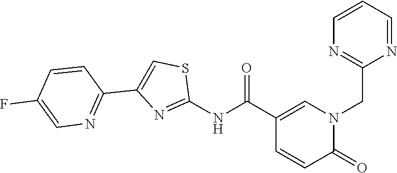 [SMILES: O=C(Nc1nc(-c2ccc(F)cn2)cs1)c1ccc(=O)n(Cc2ncccn2)c1] [symbol]
1551651-30-9 [symbol] 1270918-40-9 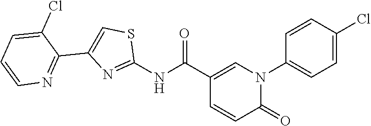 [SMILES: O=C(Nc1nc(-c2ncccc2Cl)cs1)c1ccc(=O)n(-c2ccc(Cl)cc2)c1] [symbol]
2150665-06-6 [symbol] 1283574-55-9 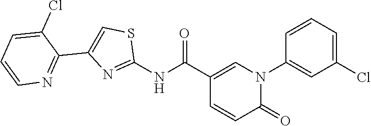 [SMILES: O=C(Nc1nc(-c2ncccc2Cl)cs1)c1ccc(=O)n(-c2cccc(Cl)c2)c1] [symbol]
2150665-06-6 [symbol] 1284635-72-8 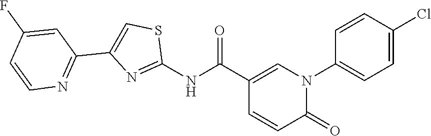 [SMILES: O=C(Nc1nc(-c2cc(F)ccn2)cs1)c1ccc(=O)n(-c2ccc(Cl)cc2)c1] [symbol]
2228222-84-0 [symbol] 1283574-55-9 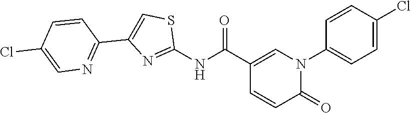 [SMILES: O=C(Nc1nc(-c2ccc(Cl)cn2)cs1)c1ccc(=O)n(-c2ccc(Cl)cc2)c1] [symbol]
1165315-17-6 [symbol] 1283574-55-9 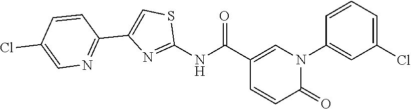 [SMILES: O=C(Nc1nc(-c2ccc(Cl)cn2)cs1)c1ccc(=O)n(-c2cccc(Cl)c2)c1] [symbol]
1185315-17-6 [symbol] 1284635-72-8 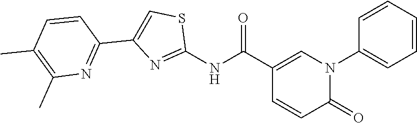 [SMILES: Cc1ccc(-c2csc(NC(=O)c3ccc(=O)n(-c4ccccc4)c3)n2)nc1C] [symbol]
1934459-93-4 [symbol] 1189982-99-7 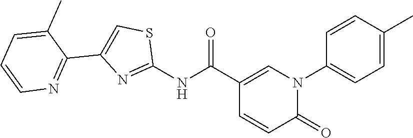 [SMILES: Cc1ccc(-n2cc(C(=O)Nc3nc(-c4ncccc4C)cs3)ccc2=O)cc1] [symbol]
1044269-91-1 [symbol] 1228275-06-6 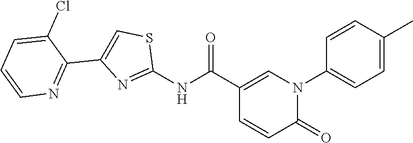 [SMILES: Cc1ccc(-n2cc(C(=O)Nc3nc(-c4ncccc4Cl)cs3)ccc2=O)cc1] [symbol]
2150665-06-6 [symbol] 1228275-06-6 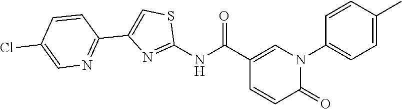 [SMILES: Cc1ccc(-n2cc(C(=O)Nc3nc(-c4ccc(Cl)cn4)cs3)ccc2=O)cc1] [symbol]
1185315-17-6 [symbol] 1228275-06-6 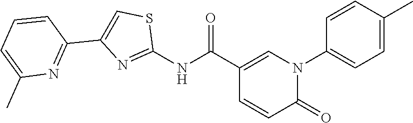 [SMILES: Cc1ccc(-n2cc(C(=O)Nc3nc(-c4cccc(C)n4)cs3)ccc2=O)cc1] [symbol]
99358-99-3 [symbol] 1228275-06-6 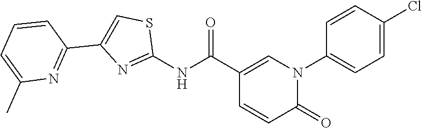 [SMILES: Cc1cccc(-c2csc(NC(=O)c3ccc(=O)n(-c4ccc(Cl)cc4)c3)n2)n1] [symbol]
99358-99-3 [symbol] 1283574-55-9 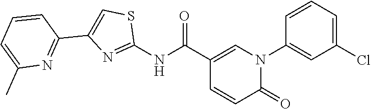 [SMILES: Cc1cccc(-c2csc(NC(=O)c3ccc(=O)n(-c4cccc(Cl)c4)c3)n2)n1] [symbol]
99358-99-3 [symbol] 1284635-72-8 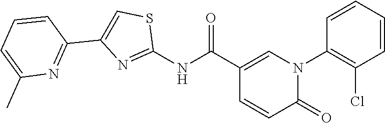 [SMILES: Cc1cccc(-c2csc(NC(=O)c3ccc(=O)n(-c4ccccc4Cl)c3)n2)n1] [symbol]
99358-99-3 [symbol] 1283545-16-3 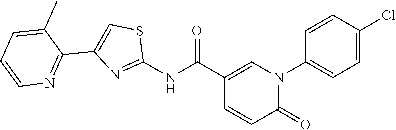 [SMILES: Cc1cccnc1-c1csc(NC(=O)c2ccc(=O)n(-c3ccc(Cl)cc3)c2)n1] [symbol]
1044269-91-1 [symbol] 1283574-55-9 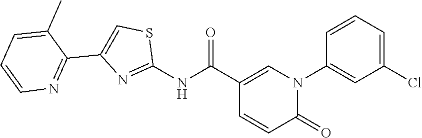 [SMILES: Cc1cccnc1-c1csc(NC(=O)c2ccc(=O)n(-c3cccc(Cl)c3)c2)n1] [symbol]
1044269-91-1 [symbol] 1264635-72-8 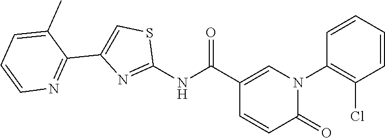 [SMILES: Cc1cccnc1-c1csc(NC(=O)c2ccc(=O)n(-c3ccccc3Cl)c2)n1] [symbol]
1044269-91-1 [symbol] 1283545-16-3 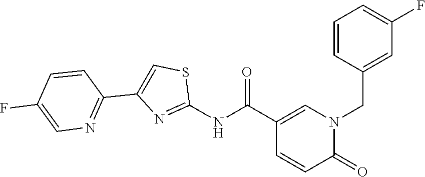 [SMILES: O=C(Nc1nc(-c2ccc(F)cn2)cs1)c1ccc(=O)n(Cc2cccc(F)c2)c1] [symbol]
1551651-30-9 [symbol] 1036437-95-2 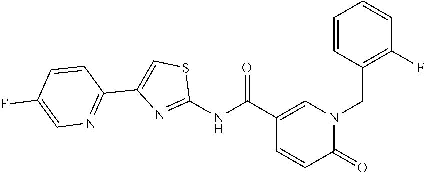 [SMILES: O=C(Nc1nc(-c2ccc(F)cn2)cs1)c1ccc(=O)n(Cc2ccccc2F)c1] [symbol]
1551651-30-9 [symbol] 941869-22-3 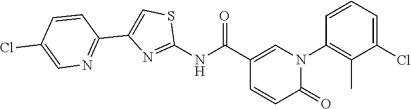 [SMILES: Cc1c(Cl)cccc1-n1cc(C(=O)Nc2nc(-c3ccc(Cl)cn3)cs2)ccc1=O] [symbol]
1185315-17-6 [symbol] 1284742-96-6 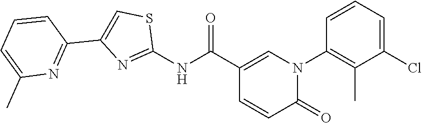 [SMILES: Cc1cccc(-c2csc(NC(=O)c3ccc(=O)n(-c4cccc(Cl)c4C)c3)n2)n1] [symbol]
99358-99-3 [symbol] 1234742-96-6 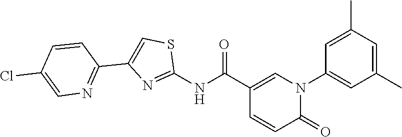 [SMILES: Cc1cc(C)cc(-n2cc(C(=O)Nc3nc(-c4ccc(Cl)cn4)cs3)ccc2=O)c1] [symbol]
1185315-17-6 [symbol] 1281268-05-1 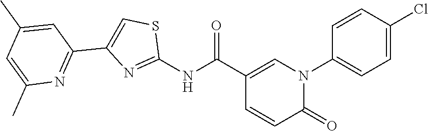 [SMILES: Cc1cc(C)nc(-c2csc(NC(=O)c3ccc(=O)n(-c4ccc(Cl)cc4)c3)n2)c1] [symbol]
1865056-88-7 [symbol] 1283574-55-9 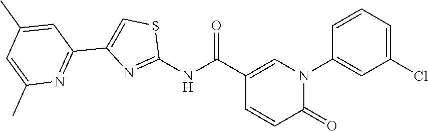 [SMILES: Cc1cc(C)nc(-c2csc(NC(=O)c3ccc(=O)n(-c4cccc(Cl)c4)c3)n2)c1] [symbol]
1865056-88-7 [symbol] 1284635-72-8 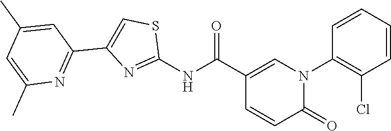 [SMILES: Cc1cc(C)nc(-c2csc(NC(=O)c3ccc(=O)n(-c4ccccc4Cl)c3)n2)c1] [symbol]
1865056-88-7 [symbol] 1283545-16-3 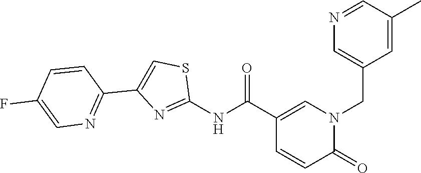 [SMILES: Cc1cncc(Cn2cc(C(=O)Nc3nc(-c4ccc(F)cn4)cs3)ccc2=O)c1] [symbol]
1551651-30-9 [symbol] 1972772-12-5 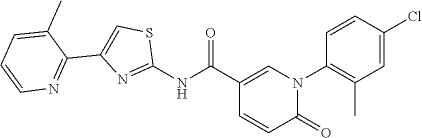 [SMILES: Cc1cc(Cl)ccc1-n1cc(C(=O)Nc2nc(-c3ncccc3C)cs2)ccc1=O] [symbol]
1044269-91-1 [symbol] 1283666-10-3 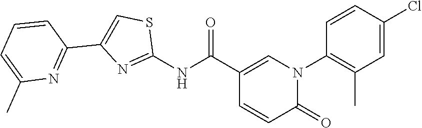 [SMILES: Cc1cccc(-c2csc(NC(=O)c3ccc(=O)n(-c4ccc(Cl)cc4C)c3)n2)n1] [symbol]
99358-99-3 [symbol] 1283666-10-3 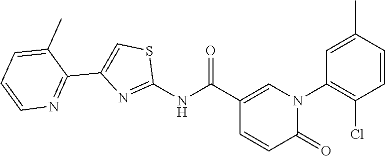 [SMILES: Cc1ccc(Cl)c(-n2cc(C(=O)Nc3nc(-c4ncccc4C)cs3)ccc2=O)c1] [symbol]
1044269-91-1 [symbol] 1930756-70-9 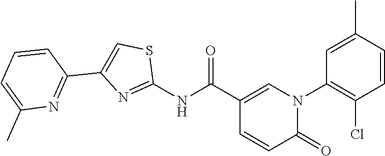 [SMILES: Cc1ccc(Cl)c(-n2cc(C(=O)Nc3nc(-c4cccc(C)n4)cs3)ccc2=O)c1] [symbol]
99358-99-3 [symbol] 1930756-70-9 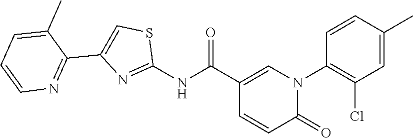 [SMILES: Cc1ccc(-n2cc(C(=O)Nc3nc(-c4ncccc4C)cs3)ccc2=O)c(Cl)c1] [symbol]
1044269-91-1 [symbol] 1282496-09-6 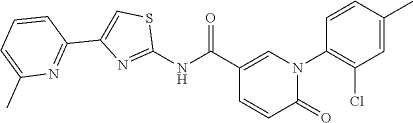 [SMILES: Cc1ccc(-n2cc(C(=O)Nc3nc(-c4cccc(C)n4)cs3)ccc2=O)c(Cl)c1] [symbol]
99358-99-3 [symbol] 1282496-09-6 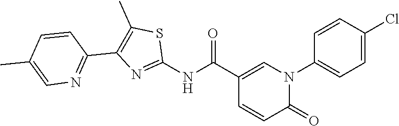 [SMILES: Cc1ccc(-c2nc(NC(=O)c3ccc(=O)n(-c4ccc(Cl)cc4)c3)sc2C)nc1] [symbol]
2294596-48-6 [symbol] 1283574-55-9 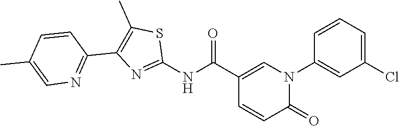 [SMILES: Cc1ccc(-c2nc(NC(=O)c3ccc(=O)n(-c4cccc(Cl)c4)c3)sc2C)nc1] [symbol]
2294596-48-6 [symbol] 1284635-72-8 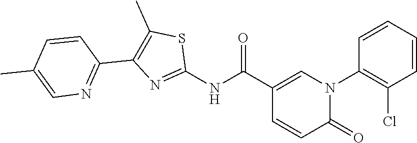 [SMILES: Cc1ccc(-c2nc(NC(=O)c3ccc(=O)n(-c4ccccc4Cl)c3)sc2C)nc1] [symbol]
2294596-48-6 [symbol] 1283545-16-3 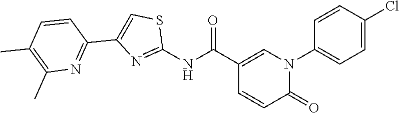 [SMILES: Cc1ccc(-c2csc(NC(=O)c3ccc(=O)n(-c4ccc(Cl)cc4)c3)n2)nc1C] [symbol]
1934459-93-4 [symbol] 1283574-55-9 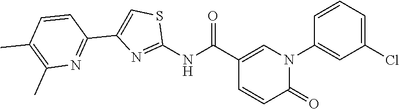 [SMILES: Cc1ccc(-c2csc(NC(=O)c3ccc(=O)n(-c4cccc(Cl)c4)c3)n2)nc1C] [symbol]
1934459-93-4 [symbol] 1284635-72-8 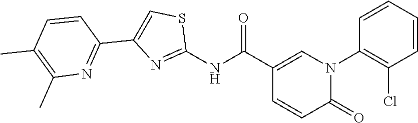 [SMILES: Cc1ccc(-c2csc(NC(=O)c3ccc(=O)n(-c4ccccc4Cl)c3)n2)nc1C] [symbol]
1934459-93-4 [symbol] 1263545-16-3 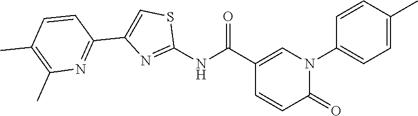 [symbol]
1934459-93-4 [symbol] 1228275-06-6 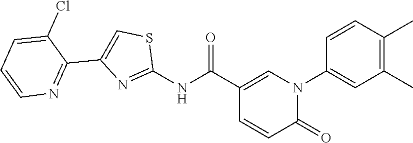 [SMILES: Cc1ccc(-n2cc(C(=O)Nc3nc(-c4ncccc4Cl)cs3)ccc2=O)cc1C] [symbol]
2150665-06-6 [symbol] 1282073-48-6 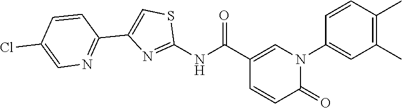 [SMILES: Cc1ccc(-n2cc(C(=O)Nc3nc(-c4ccc(Cl)cn4)cs3)ccc2=O)cc1C] [symbol]
1185315-17-6 [symbol] 1282073-48-6 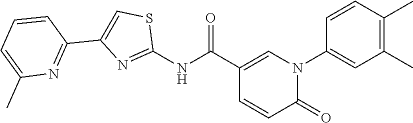 [SMILES: Cc1cccc(-c2csc(NC(=O)c3ccc(=O)n(-c4ccc(C)c(C)c4)c3)n2)n1] [symbol]
99358-99-3 [symbol] 1282073-48-6 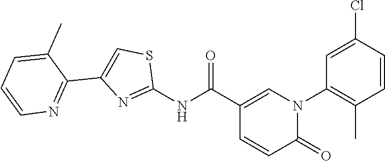 [SMILES: Cc1ccc(Cl)cc1-n1cc(C(=O)Nc2nc(-c3ncccc3C)cs2)ccc1=O] [symbol]
1044269-91-1 [symbol] 1285116-17-7 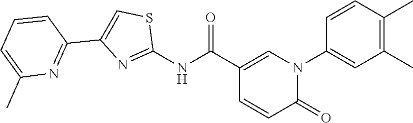 [SMILES: Cc1cccc(-c2csc(NC(=O)c3ccc(=O)n(-c4ccc(C)c(C)c4)c3)n2)n1] [symbol]
99358-99-3 [symbol] 1285116-17-7 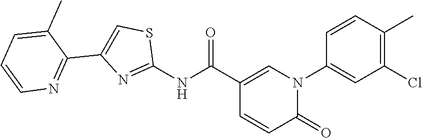 [SMILES: Cc1ccc(-n2cc(C(=O)Nc3nc(-c4ncccc4C)cs3)ccc2=O)cc1Cl] [symbol]
1044269-91-1 [symbol] 1285568-37-7 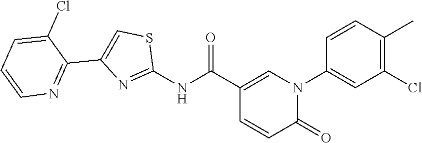 [SMILES: Cc1ccc(-n2cc(C(=O)Nc3nc(-c4ncccc4Cl)cs3)ccc2=O)cc1Cl] [symbol]
2150665-06-6 [symbol] 1285568-37-7 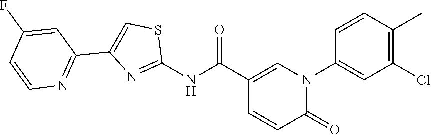 [SMILES: Cc1ccc(-n2cc(C(=O)Nc3nc(-c4cc(F)ccn4)cs3)ccc2=O)cc1Cl] [symbol]
2228222-84-0 [symbol] 1285568-37-7 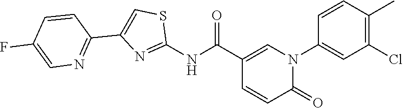 [SMILES: Cc1ccc(-n2cc(C(=O)Nc3nc(-c4ccc(F)cn4)cs3)ccc2=O)cc1Cl] [symbol]
1551651-30-9 [symbol] 1285568-37-7 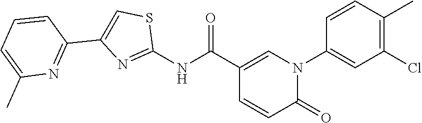 [SMILES: Cc1cccc(-c2csc(NC(=O)c3ccc(=O)n(-c4ccc(C)c(Cl)c4)c3)n2)n1] [symbol]
99358-99-3 [symbol] 1285568-37-7 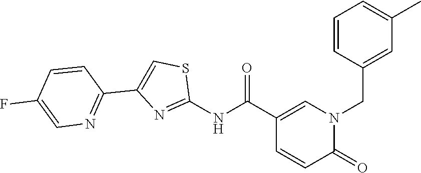 [SMILES: Cc1cccc(Cn2cc(C(=O)Nc3nc(-c4ccc(F)cn4)cs3)ccc2=O)c1] [symbol]
1551651-30-9 [symbol] 1036564-02-9 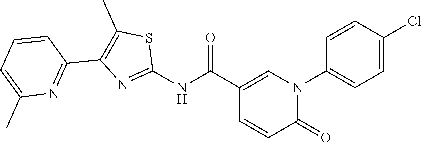 [SMILES: Cc1cccc(-c2nc(NC(=O)c3ccc(=O)n(-c4ccc(Cl)cc4)c3)sc2C)n1] [symbol]
2169472-77-7 [symbol] 1283574-55-9 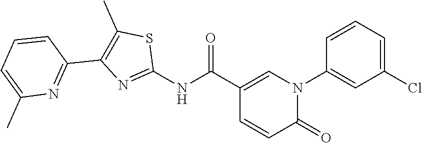 [SMILES: Cc1cccc(-c2nc(NC(=O)c3ccc(=O)n(-c4cccc(Cl)c4)c3)sc2C)n1] [symbol]
2169472-77-7 [symbol] 1284635-72-8 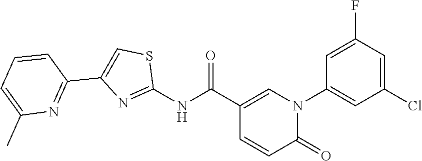 [SMILES: Cc1cccc(-c2csc(NC(=O)c3ccc(=O)n(-c4cc(F)cc(Cl)c4)c3)n2)n1] [symbol]
99358-99-3 [symbol] 1989519-81-4 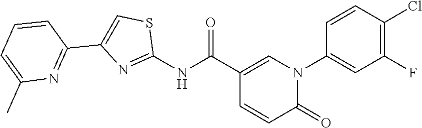 [SMILES: Cc1cccc(-c2csc(NC(=O)c3ccc(=O)n(-c4ccc(Cl)c(F)c4)c3)n2)n1] [symbol]
99358-99-3 [symbol] 1285009-59-7 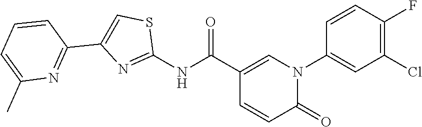 [SMILES: Cc1cccc(-c2csc(NC(=O)c3ccc(=O)n(-c4ccc(F)c(Cl)c4)c3)n2)n1] [symbol]
99358-99-3 [symbol] 1285329-31-8 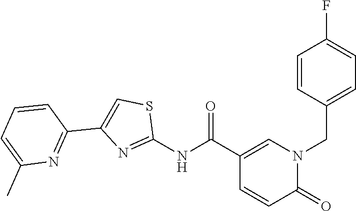 [SMILES: Cc1cccc(-c2csc(NC(=O)c3ccc(=O)n(Cc4ccc(F)cc4)c3)n2)n1] [symbol]
99358-99-3 [symbol] 941809-20-1 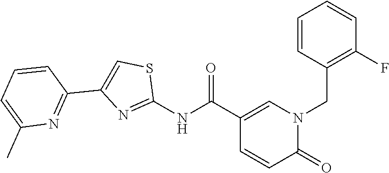 [SMILES: Cc1cccc(-c2csc(NC(=O)c3ccc(=O)n(Cc4ccccc4F)c3)n2)n1] [symbol]
99358-99-3 [symbol] 941869-22-3 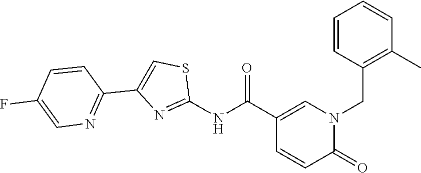 [SMILES: Cc1ccccc1Cn1cc(C(=O)Nc2nc(-c3ccc(F)cn3)cs2)ccc1=O] [symbol]
1551651-30-9 [symbol] 1036495-60-9 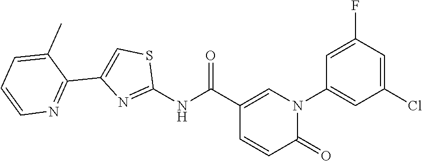 [SMILES: Cc1cccnc1-c1csc(NC(=O)c2ccc(=O)n(-c3cc(F)cc(Cl)c3)c2)n1] [symbol]
1044269-91-1 [symbol] 1989519-81-4 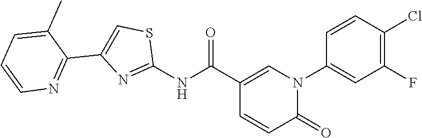 [SMILES: Cc1cccnc1-c1csc(NC(=O)c2ccc(=O)n(-c3ccc(Cl)c(F)c3)c2)n1] [symbol]
1044269-91-1 [symbol] 1285009-59-7 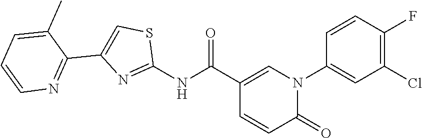 [SMILES: Cc1cccnc1-c1csc(NC(=O)c2ccc(=O)n(-c3ccc(F)c(Cl)c3)c2)n1] [symbol]
1044269-91-1 [symbol] 1285329-31-8 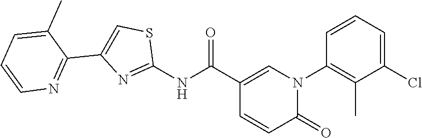 [SMILES: Cc1cccnc1-c1csc(NC(=O)c2ccc(=O)n(-c3cccc(Cl)c3C)c2)n1] [symbol]
1044269-91-1 [symbol] 1284742-96-6 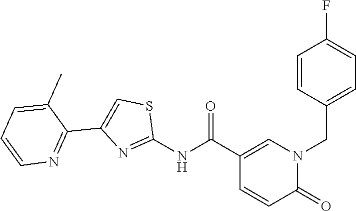 [SMILES: Cc1cccnc1-c1csc(NC(=O)c2ccc(=O)n(Cc3ccc(F)cc3)c2)n1] [symbol]
1044269-91-1 [symbol] 941669-20-1 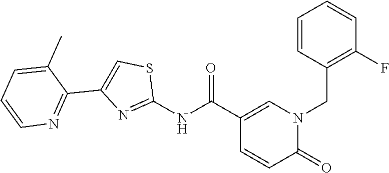 [SMILES: Cc1cccnc1-c1csc(NC(=O)c2ccc(=O)n(Cc3ccccc3F)c2)n1] [symbol]
1044269-91-1 [symbol] 941869-22-3 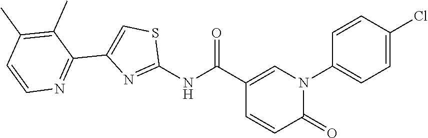 [SMILES: Cc1ccnc(-c2csc(NC(=O)c3ccc(=O)n(-c4ccc(Cl)cc4)c3)n2)c1C] [symbol]
2189166-95-2 [symbol] 1283574-55-9 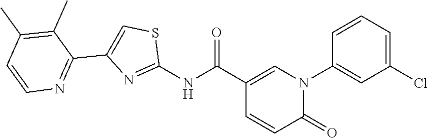 [SMILES: Cc1ccnc(-c2csc(NC(=O)c3ccc(=O)n(-c4cccc(Cl)c4)c3)n2)c1C] [symbol]
2109186-95-2 [symbol] 1284635-72-8 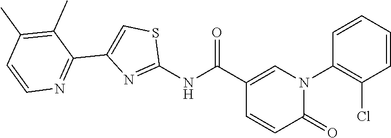 [SMILES: Cc1ccnc(-c2csc(NC(=O)c3ccc(=O)n(-c4ccccc4Cl)c3)n2)c1C] [symbol]
2169166-95-2 [symbol] 1283545-16-3 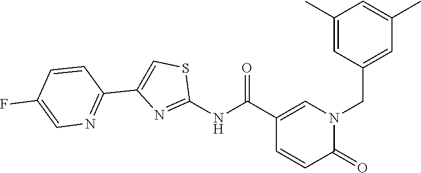 [SMILES: Cc1cc(C)cc(Cn2cc(C(=O)Nc3nc(-c4ccc(F)cn4)cs3)ccc2=O)c1] [symbol]
1551651-30-9 [symbol] 1178479-80-5 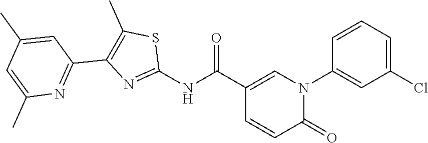 [SMILES: Cc1cc(C)nc(-c2nc(NC(=O)c3ccc(=O)n(-c4cccc(Cl)c4)c3)sc2C)c1] [symbol]
2287501-11-3 [symbol] 1284635-72-8 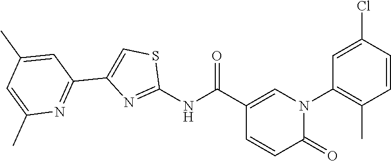 [SMILES: Cc1cc(C)nc(-c2csc(NC(=O)c3ccc(=O)n(-c4cc(Cl)ccc4C)c3)n2)c1] [symbol]
1865056-88-7 [symbol] 1285116-17-7 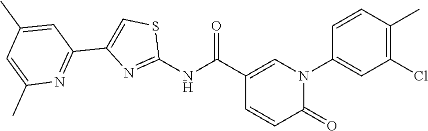 [symbol]
1865056-88-7 [symbol] 1285568-37-7 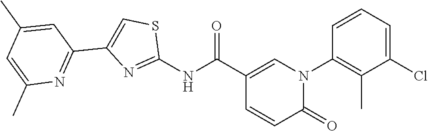 [SMILES: Cc1cc(C)nc(-c2csc(NC(=O)c3ccc(=O)n(-c4cccc(Cl)c4C)c3)n2)c1] [symbol]
1865056-88-7 [symbol] 1284742-96-6 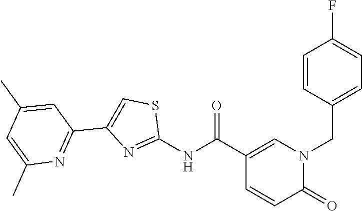 [SMILES: Cc1cc(C)nc(-c2csc(NC(=O)c3ccc(=O)n(Cc4ccc(F)cc4)c3)n2)c1] [symbol]
1865056-86-7 [symbol] 941869-20-1 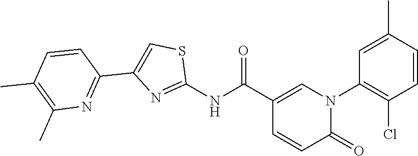 [SMILES: Cc1ccc(Cl)c(-n2cc(C(=O)Nc3nc(-c4ccc(C)c(C)n4)cs3)ccc2=O)c1] [symbol]
1934459-93-4 [symbol] 1930756-70-9 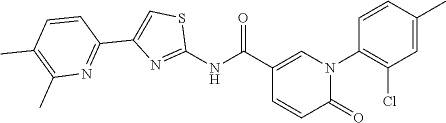 [symbol]
1934459-93-4 [symbol] 1282496-09-6 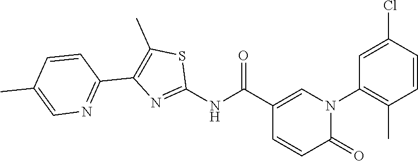 [SMILES: Cc1ccc(-c2nc(NC(=O)c3ccc(=O)n(-c4cc(Cl)ccc4C)c3)sc2C)nc1] [symbol]
2294596-48-6 [symbol] 1285116-17-7 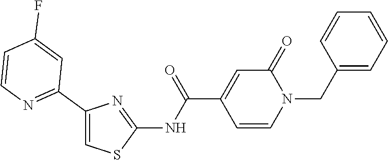 [SMILES: O=C(Nc1nc(-c2cc(F)ccn2)cs1)c1ccn(Cc2ccccc2)c(=O)c1] [symbol]
2228222-84-0 [symbol] 63987-74-6 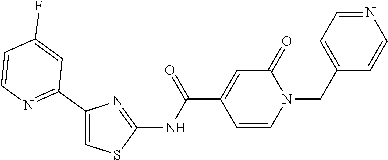 [SMILES: O=C(Nc1nc(-c2cc(F)ccn2)cs1)c1ccn(Cc2ccncc2)c(=O)c1] [symbol]
2228222-84-0 [symbol] 1555938-14-1 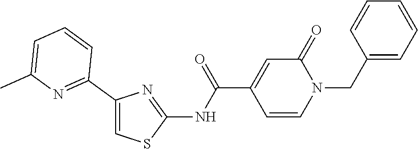 [SMILES: Cc1cccc(-c2csc(NC(=O)c3ccn(Cc4ccccc4)c(=O)c3)n2)n1] [symbol]
99358-99-3 [symbol] 63987-74-6 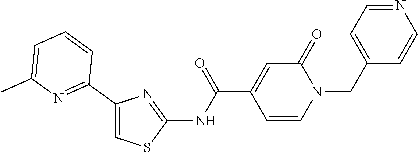 [SMILES: Cc1cccc(-c2csc(NC(=O)c3ccn(Cc4ccncc4)c(=O)c3)n2)n1] [symbol]
99358-99-3 [symbol] 1555938-14-1 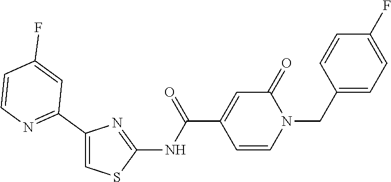 [SMILES: O=C(Nc1nc(-c2cc(F)ccn2)cs1)c1ccn(Cc2ccc(F)cc2)c(=O)c1] [symbol]
2228222-84-0 [symbol] 1398927-27-8 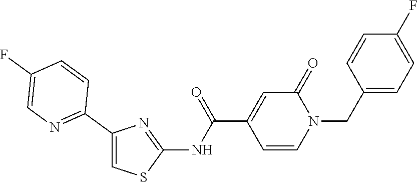 [SMILES: O=C(Nc1nc(-c2ccc(F)cn2)cs1)c1ccn(Cc2ccc(F)cc2)c(=O)c1] [symbol]
1551651-30-9 [symbol] 1368927-27-8 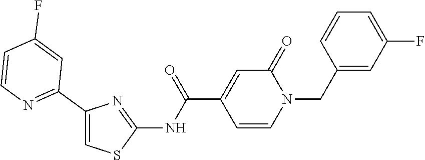 [SMILES: O=C(Nc1nc(-c2cc(F)ccn2)cs1)c1ccn(Cc2cccc(F)c2)c(=O)c1] [symbol]
2228222-84-0 [symbol] 1557594-14-0 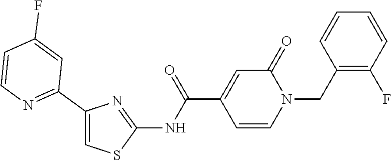 [SMILES: O=C(Nc1nc(-c2cc(F)ccn2)cs1)c1ccn(Cc2ccccc2F)c(=O)c1] [symbol]
2228222-84-0 [symbol] 1368471-21-9 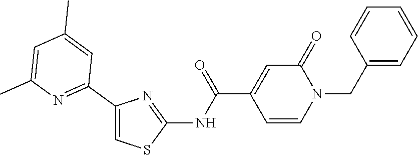 [SMILES: Cc1cc(C)nc(-c2csc(NC(=O)c3ccn(Cc4ccccc4)c(=O)c3)n2)c1] [symbol]
1865056-88-7 [symbol] 63987-74-6 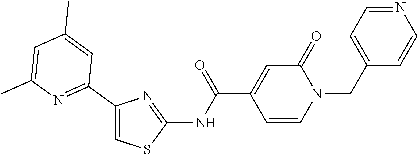 [SMILES: Cc1cc(C)nc(-c2csc(NC(=O)c3ccn(Cc4ccncc4)c(=O)c3)n2)c1] [symbol]
1865056-88-7 [symbol] 1555938-14-1 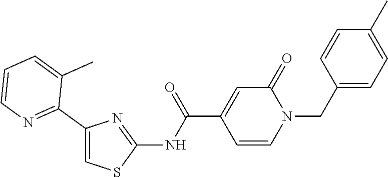 [SMILES: Cc1ccc(Cn2ccc(C(=O)Nc3nc(-c4ncccc4C)cs3)cc2=O)cc1] [symbol]
1044269-91-1 [symbol] 1203544-05-1 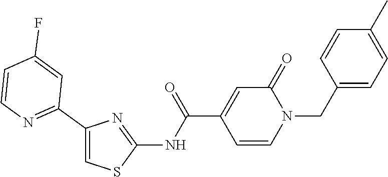 [SMILES: Cc1ccc(Cn2ccc(C(=O)Nc3nc(-c4cc(F)ccn4)cs3)cc2=O)cc1] [symbol]
2228222-84-0 [symbol] 1203544-05-1 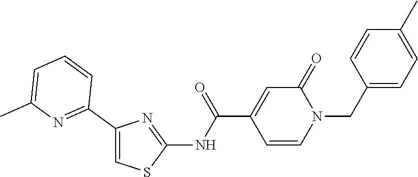 [symbol]
99358-99-3 [symbol] 1203544-05-1 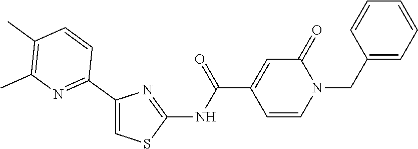 [SMILES: Cc1ccc(-c2csc(NC(=O)c3ccn(Cc4ccccc4)c(=O)c3)n2)nc1C] [symbol]
1934459-93-4 [symbol] 63987-74-6 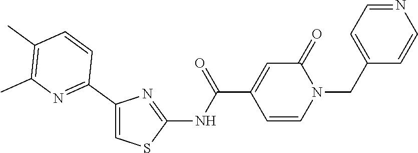 [SMILES: Cc1ccc(-c2csc(NC(=O)c3ccn(Cc4ccncc4)c(=O)c3)n2)nc1C] [symbol]
1934459-93-4 [symbol] 1555938-14-1 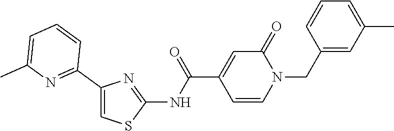 [symbol]
99358-99-3 [symbol] 1369077-49-5 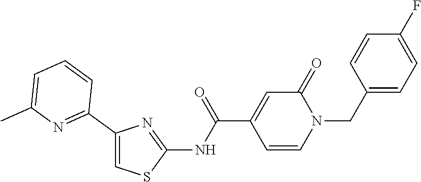 [SMILES: Cc1cccc(-c2csc(NC(=O)c3ccn(Cc4ccc(F)cc4)c(=O)c3)n2)n1] [symbol]
99358-99-3 [symbol] 1368927-27-6 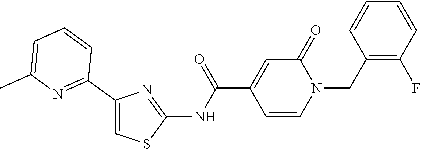 [SMILES: Cc1cccc(-c2csc(NC(=O)c3ccn(Cc4ccccc4F)c(=O)c3)n2)n1] [symbol]
99358-99-3 [symbol] 1368471-21-9 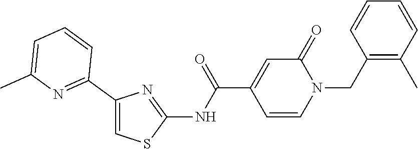 [symbol]
99358-99-3 [symbol] 1368768-57-3 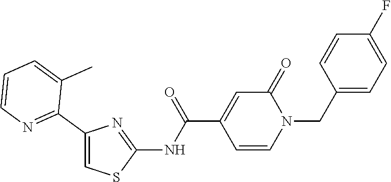 [SMILES: Cc1cccnc1-c1csc(NC(=O)c2ccn(Cc3ccc(F)cc3)c(=O)c2)n1] [symbol]
1044269-91-1 [symbol] 1368927-27-8 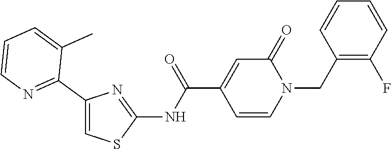 [SMILES: Cc1cccnc1-c1csc(NC(=O)c2ccn(Cc3ccccc3F)c(=O)c2)n1] [symbol]
1044269-91-1 [symbol] 1368471-21-9 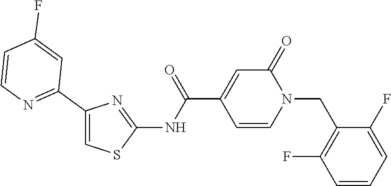 [SMILES: O=C(Nc1nc(-c2cc(F)ccn2)cs1)c1ccn(Cc2c(F)cccc2F)c(=O)c1] [symbol]
2228222-84-0 [symbol] 1773667-76-7 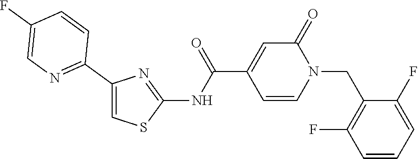 [SMILES: O=C(Nc1nc(-c2ccc(F)cn2)cs1)c1ccn(Cc2c(F)cccc2F)c(=O)c1] [symbol]
1551651-30-9 [symbol] 1773667-76-7 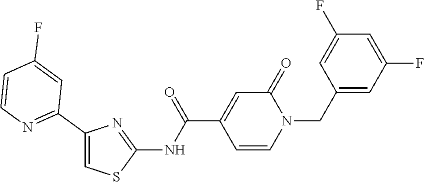 [SMILES: O=C(Nc1nc(-c2cc(F)ccn2)cs1)c1ccn(Cc2cc(F)cc(F)c2)c(=O)c1] [symbol]
2228222-84-0 [symbol] 1983293-83-9 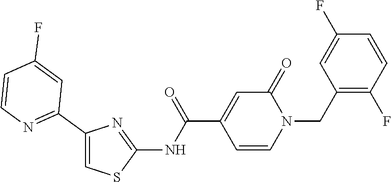 [SMILES: O=C(Nc1nc(-c2cc(F)ccn2)cs1)c1ccn(Cc2cc(F)ccc2F)c(=O)c1] [symbol]
2228222-64-0 [symbol] 1545891-16-4 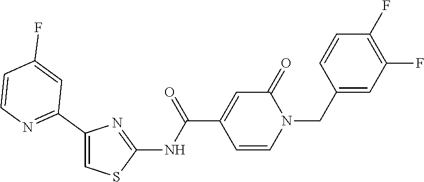 [SMILES: O=C(Nc1nc(-c2cc(F)ccn2)cs1)c1ccn(Cc2ccc(F)c(F)c2)c(=O)c1] [symbol]
2228222-84-0 [symbol] 1544725-62-3 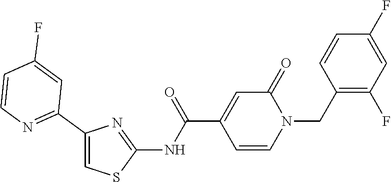 [SMILES: O=C(Nc1nc(-c2cc(F)ccn2)cs1)c1ccn(Cc2ccc(F)cc2F)c(=O)c1] [symbol]
2228222-84-0 [symbol] 1550842-35-7 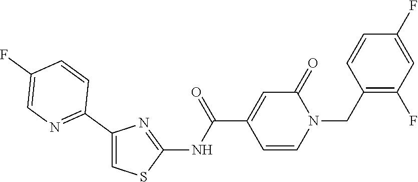 [SMILES: O=C(Nc1nc(-c2ccc(F)cn2)cs1)c1ccn(Cc2ccc(F)cc2F)c(=O)c1] [symbol]
1551651-30-9 [symbol] 1550842-35-7 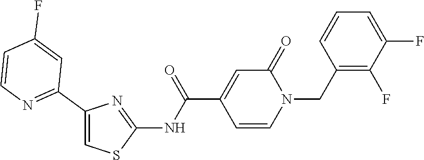 [SMILES: O=C(Nc1nc(-c2cc(F)ccn2)cs1)c1ccn(Cc2cccc(F)c2F)c(=O)c1] [symbol]
2228222-84-0 [symbol] 1557687-92-9 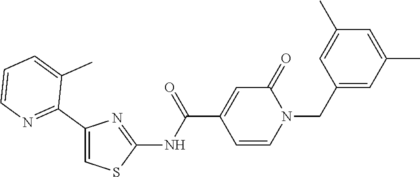 [SMILES: Cc1cc(C)cc(Cn2ccc(C(=O)Nc3nc(-c4ncccc4C)cs3)cc2=O)c1] [symbol]
1044269-91-1 [symbol] 1557687-80-1 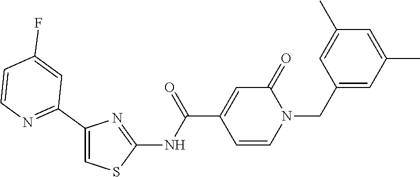 [SMILES: Cc1cc(C)cc(Cn2ccc(C(=O)Nc3nc(-c4cc(F)ccn4)cs3)cc2=O)c1] [symbol]
2228222-84-0 [symbol] 1557687-60-1 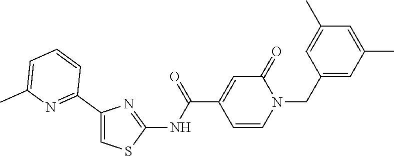 [symbol]
99358-99-3 [symbol] 1557687-60-1 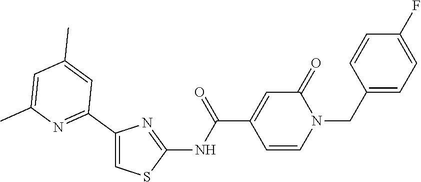 [SMILES: Cc1cc(C)nc(-c2csc(NC(=O)c3ccn(Cc4ccc(F)cc4)c(=O)c3)n2)c1] [symbol]
1885056-88-7 [symbol] 1368927-27-8 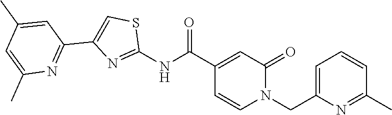 [symbol]
1865056-88-7 [symbol] 1548114-14-2 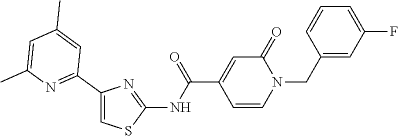 [SMILES: Cc1cc(C)nc(-c2csc(NC(=O)c3ccn(Cc4cccc(F)c4)c(=O)c3)n2)c1] [symbol]
1865056-88-7 [symbol] 1557694-14-0 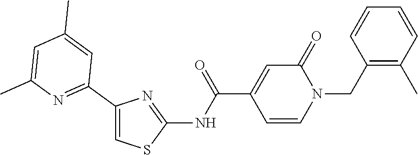 [symbol]
1865056-88-7 [symbol] 1368788-57-3 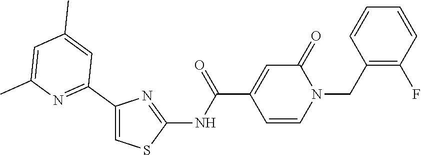 [SMILES: Cc1cc(C)nc(-c2csc(NC(=O)c3ccn(Cc4ccccc4F)c(=O)c3)n2)c1] [symbol]
1665056-68-7 [symbol] 1368471-21-9 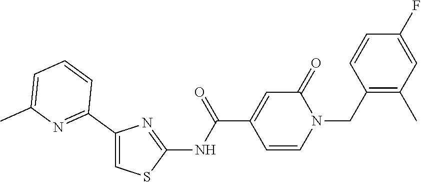 [symbol]
99358-99-3 [symbol] 1778510-78-3 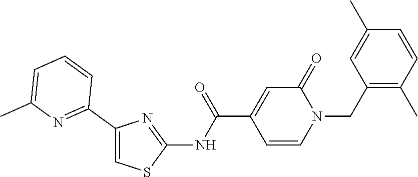 [symbol]
99358-99-3 [symbol] 1368576-28-6 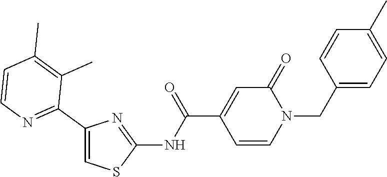 [SMILES: Cc1ccc(Cn2ccc(C(=O)Nc3nc(-c4nccc(C)c4C)cs3)cc2=O)cc1] [symbol]
2169186-95-2 [symbol] 1203544-05-1 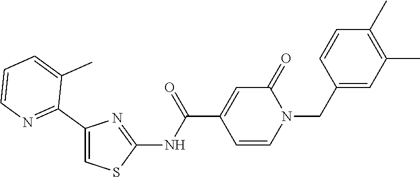 [SMILES: Cc1ccc(Cn2ccc(C(=O)Nc3nc(-c4ncccc4C)cs3)cc2=O)cc1C] [symbol]
1044269-91-1 [symbol] 1557693-99-8 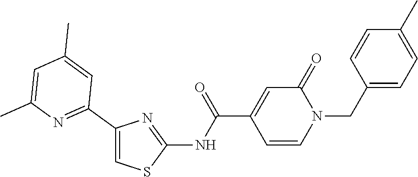 [symbol]
1865056-88-7 [symbol] 1203544-05-1 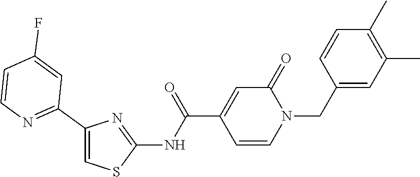 [SMILES: Cc1ccc(Cn2ccc(C(=O)Nc3nc(-c4cc(F)ccn4)cs3)cc2=O)cc1C] [symbol]
2228222-84-0 [symbol] 1557693-99-8 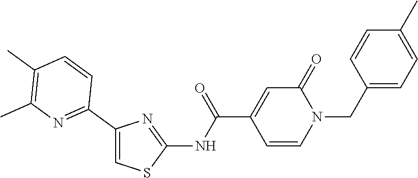 [symbol]
1934459-93-4 [symbol] 1203544-05-1 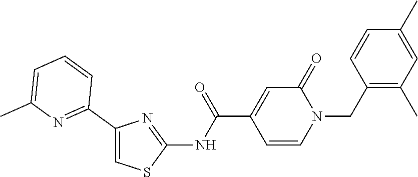 [symbol]
99358-99-3 [symbol] 1368673-86-2 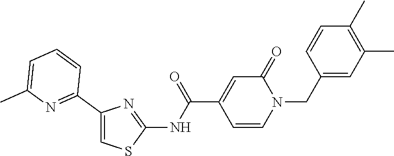 [symbol]
99358-99-3 [symbol] 1557693-99-8 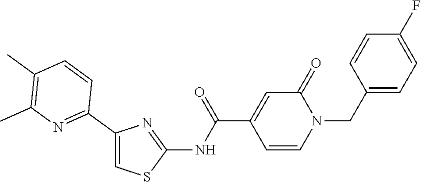 [SMILES: Cc1ccc(-c2csc(NC(=O)c3ccn(Cc4ccc(F)cc4)c(=O)c3)n2)nc1C] [symbol]
1934459-93-4 [symbol] 1368927-27-8 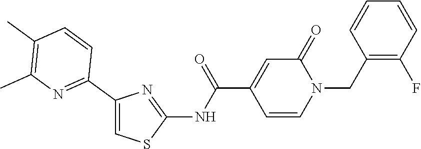 [SMILES: Cc1ccc(-c2csc(NC(=O)c3ccn(Cc4ccccc4F)c(=O)c3)n2)nc1C] [symbol]
1934459-93-4 [symbol] 1368471-21-9 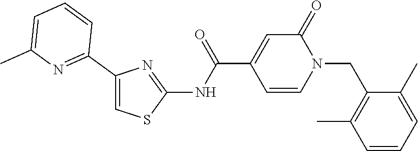 [symbol]
99358-99-3 [symbol] 1557087-82-7 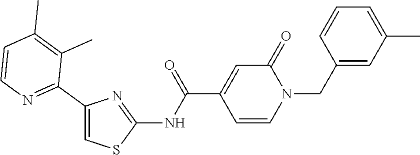 [SMILES: Cc1cccc(Cn2ccc(C(=O)Nc3nc(-c4nccc(C)c4C)cs3)cc2=O)c1] [symbol]
2169166-95-2 [symbol] 1369077-49-5 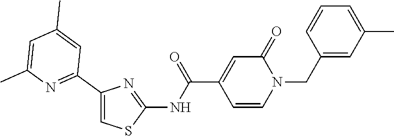 [symbol]
1865056-88-7 [symbol] 1369077-49-5 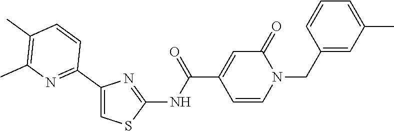 [symbol]
1934459-93-4 [symbol] 1369077-49-5 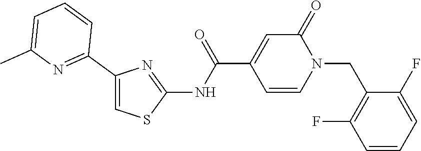 [SMILES: Cc1cccc(-c2csc(NC(=O)c3ccn(Cc4c(F)cccc4F)c(=O)c3)n2)n1] [symbol]
99358-99-3 [symbol] 1773667-76-7 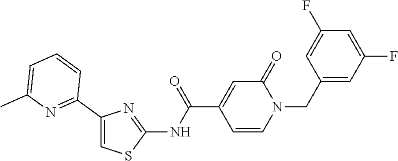 [SMILES: Cc1cccc(-c2csc(NC(=O)c3ccn(Cc4cc(F)cc(F)c4)c(=O)c3)n2)n1] [symbol]
99358-99-3 [symbol] 1983293-83-9 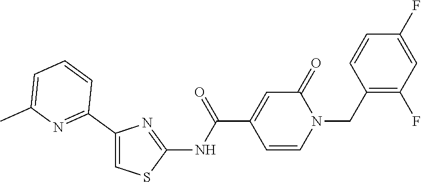 [SMILES: Cc1cccc(-c2csc(NC(=O)c3ccn(Cc4ccc(F)cc4F)c(=O)c3)n2)n1] [symbol]
99358-99-3 [symbol] 1550842-35-7 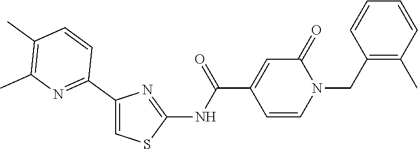 [symbol]
1934459-93-4 [symbol] 1368768-57-3 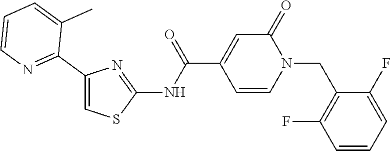 [SMILES: Cc1cccnc1-c1csc(NC(=O)c2ccn(Cc3c(F)cccc3F)c(=O)c2)n1] [symbol]
1044269-91-1 [symbol] 1773667-76-7 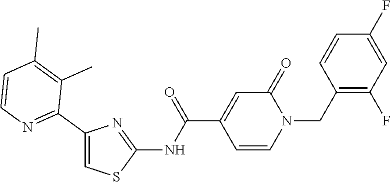 [SMILES: Cc1ccnc(-c2csc(NC(=O)c3ccn(Cc4ccc(F)cc4F)c(=O)c3)n2)c1C] [symbol]
1044289-91-1 [symbol] 1550842-35-7 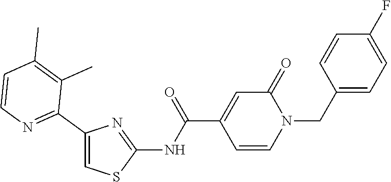 [SMILES: Cc1ccnc(-c2csc(NC(=O)c3ccn(Cc4ccc(F)cc4)c(=O)c3)n2)c1C] [symbol]
2169166-95-2 [symbol] 1368927-27-8 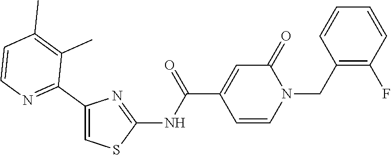 [SMILES: Cc1ccnc(-c2csc(NC(=O)c3ccn(Cc4ccccc4F)c(=O)c3)n2)c1C] [symbol]
2169166-95-2 [symbol] 1368471-21-9 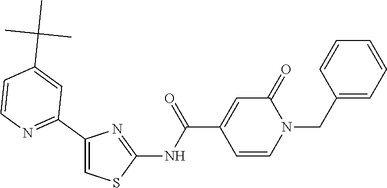 [SMILES: CC(C)(C)c1ccnc(-c2csc(NC(=O)c3ccn(Cc4ccccc4)c(=O)c3)n2)c1] [symbol]
2253868-94-7 [symbol] 63987-74-6 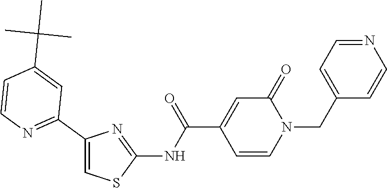 [SMILES: CC(C)(C)c1ccnc(-c2csc(NC(=O)c3ccn(Cc4ccncc4)c(=O)c3)n2)c1] [symbol]
2253868-94-7 [symbol] 1555938-14-1 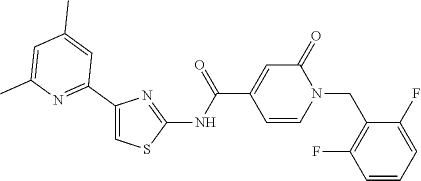 [SMILES: Cc1cc(C)nc(-c2csc(NC(=O)c3ccn(Cc4c(F)cccc4F)c(=O)c3)n2)c1] [symbol]
1865056-88-7 [symbol] 1773867-78-7 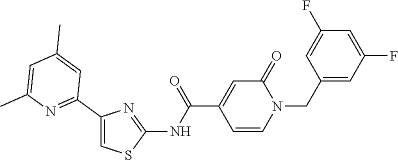 [SMILES: Cc1cc(C)nc(-c2csc(NC(=O)c3ccn(Cc4cc(F)cc(F)c4)c(=O)c3)n2)c1] [symbol]
1885056-88-7 [symbol] 1983293-83-9 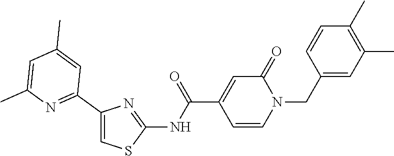 [symbol]
1865056-88-7 [symbol] 1557693-99-8 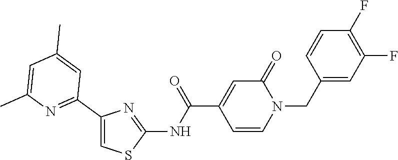 [SMILES: Cc1cc(C)nc(-c2csc(NC(=O)c3ccn(Cc4ccc(F)c(F)c4)c(=O)c3)n2)c1] [symbol]
1865056-88-7 [symbol] 1544725-62-3 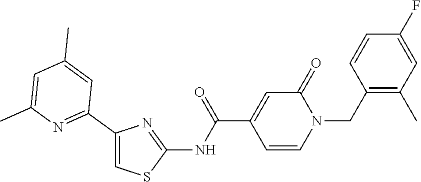 [symbol]
1865056-88-7 [symbol] 1778510-78-3 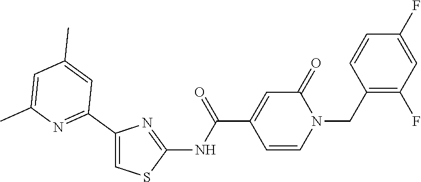 [SMILES: Cc1cc(C)nc(-c2csc(NC(=O)c3ccn(Cc4ccc(F)cc4F)c(=O)c3)n2)c1] [symbol]
1865056-86-7 [symbol] 1550842-35-7 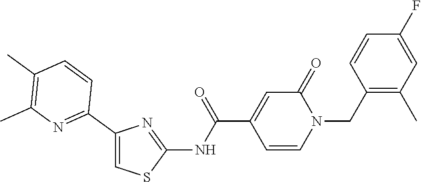 [SMILES: Cc1cc(F)ccc1Cn1ccc(C(=O)Nc2nc(-c3ccc(C)c(C)n3)cs2)cc1=O] [symbol]
1934459-93-4 [symbol] 1778510-78-3 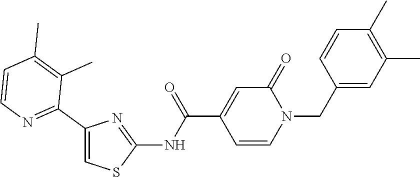 [SMILES: Cc1ccc(Cn2ccc(C(=O)Nc3nc(-c4nccc(C)c4C)cs3)cc2=O)cc1C] [symbol]
2169166-95-2 [symbol] 1557893-99-8 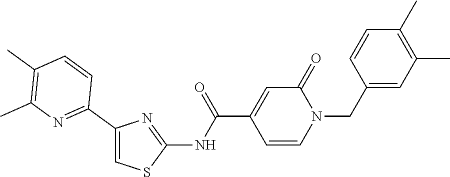 [symbol]
1934459-93-4 [symbol] 1557893-99-8 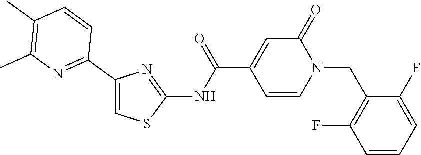 [SMILES: Cc1ccc(-c2csc(NC(=O)c3ccn(Cc4c(F)cccc4F)c(=O)c3)n2)nc1C] [symbol]
1934459-93-4 [symbol] 1773667-76-7 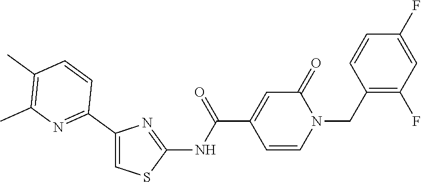 [SMILES: Cc1ccc(-c2csc(NC(=O)c3ccn(Cc4ccc(F)cc4F)c(=O)c3)n2)nc1C] [symbol]
1934459-93-4 [symbol] 1550842-35-7 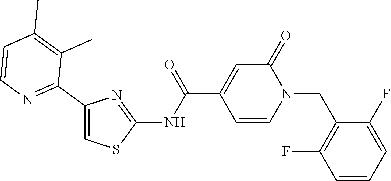 [SMILES: Cc1ccnc(-c2csc(NC(=O)c3ccn(Cc4c(F)cccc4F)c(=O)c3)n2)c1C] [symbol]
2169186-95-2 [symbol] 1773667-76-7 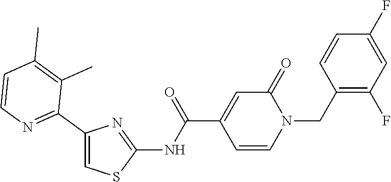 [SMILES: Cc1ccnc(-c2csc(NC(=O)c3ccn(Cc4ccc(F)cc4F)c(=O)c3)n2)c1C] [symbol]
2169166-95-2 [symbol] 1550842-35-7 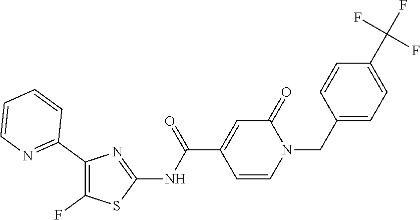 [SMILES: O=C(Nc1nc(-c2ccccn2)c(F)s1)c1ccn(Cc2ccc(C(F)(F)F)cc2)c(=O)c1] [symbol]
1044270-93-0 [symbol] 1556050-28-2 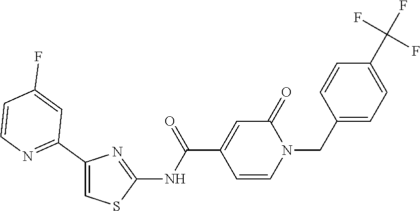 [SMILES: O=C(Nc1nc(-c2cc(F)ccn2)cs1)c1ccn(Cc2ccc(C(F)(F)F)cc2)c(=O)c1] [symbol]
2228222-84-0 [symbol] 1550050-28-2 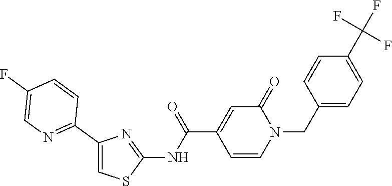 [SMILES: O=C(Nc1nc(-c2ccc(F)cn2)cs1)c1ccn(Cc2ccc(C(F)(F)F)cc2)c(=O)c1] [symbol]
1551651-30-9 [symbol] 1556050-28-2 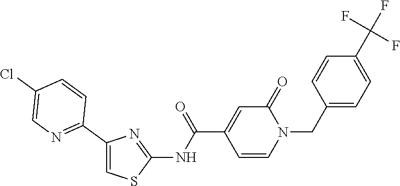 [SMILES: O=C(Nc1nc(-c2ccc(Cl)cn2)cs1)c1ccn(Cc2ccc(C(F)(F)F)cc2)c(=O)c1] [symbol]
1185315-17-6 [symbol] 1556050-28-2 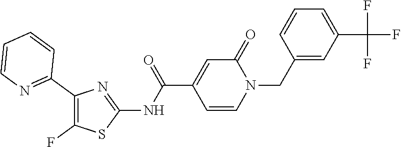 [SMILES: O=C(Nc1nc(-c2ccccn2)c(F)s1)c1ccn(Cc2cccc(C(F)(F)F)c2)c(=O)c1] [symbol]
1044270-93-0 [symbol] 1550842-99-3 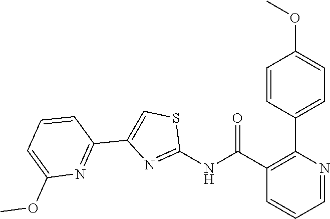 [SMILES: COc1ccc(-c2ncccc2C(=O)Nc2nc(-c3cccc(OC)n3)cs2)cc1] [symbol]
1870028-77-5 [symbol] 1225539-80-9 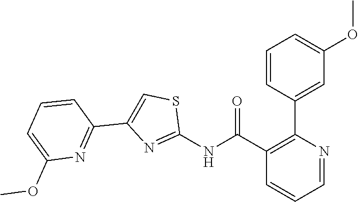 [symbol]
1870028-77-5 [symbol] 912773-03-6 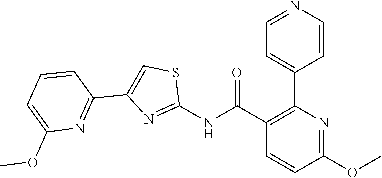 [symbol]
1870028-77-5 [symbol] 1607429-46-8 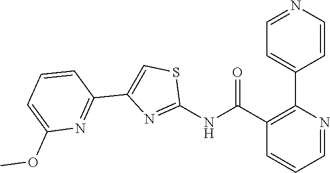 [SMILES: COc1cccc(-c2csc(NC(=O)c3cccnc3-c3ccncc3)n2)n1] [symbol]
1870028-77-5 [symbol] 1429505-78-1 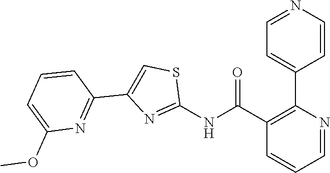 [SMILES: COc1cccc(-c2csc(NC(=O)c3cccnc3-c3ccncc3)n2)n1] [symbol]
1870028-77-5 [symbol] 1214353-01-1 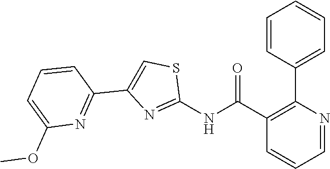 [SMILES: COc1cccc(-c2csc(NC(=O)c3cccnc3-c3ccccc3)n2)n1] [symbol]
1870028-77-5 [symbol] 33421-39-5 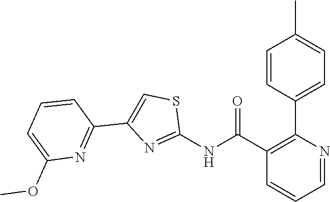 [SMILES: COc1cccc(-c2csc(NC(=O)c3cccnc3-c3ccc(C)cc3)n2)n1] [symbol]
1870028-77-5 [symbol] 1226205-68-0 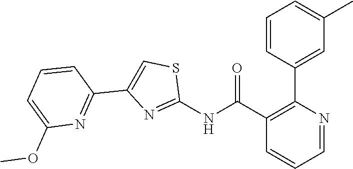 [symbol]
1870028-77-5 [symbol] 1226205-71-5 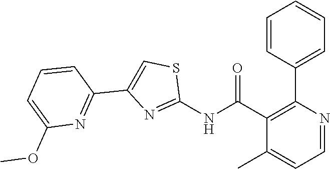 [SMILES: COc1cccc(-c2csc(NC(=O)c3c(C)ccnc3-c3ccccc3)n2)n1] [symbol]
1870028-77-5 [symbol] 58787-23-8 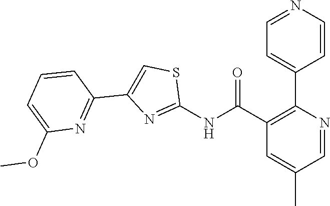 [SMILES: COc1cccc(-c2csc(NC(=O)c3cc(C)cnc3-c3ccncc3)n2)n1] [symbol]
1870028-77-5 [symbol] 1557250-92-6 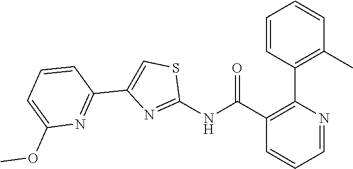 [symbol]
1870028-77-5 [symbol] 1226205-48-6 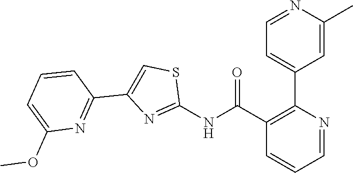 [symbol]
1870028-77-5 [symbol] 2091872-09-0 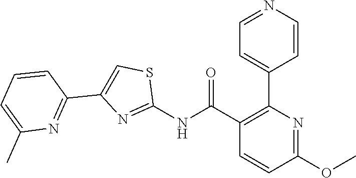 [SMILES: COc1ccc(C(=O)Nc2nc(-c3cccc(C)n3)cs2)c(-c2ccncc2)n1] [symbol]
99358-99-3 [symbol] 1607429-46-8 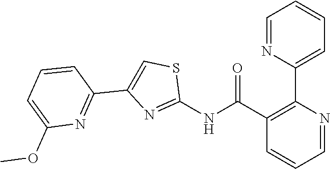 [SMILES: COc1cccc(-c2csc(NC(=O)c3cccnc3-c3ccccn3)n2)n1] [symbol]
1870028-77-5 [symbol] 220340-46-5 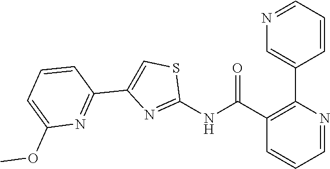 [SMILES: COc1cccc(-c2csc(NC(=O)c3cccnc3-c3cccnc3)n2)n1] [symbol]
1870028-77-5 [symbol] 851368-00-8 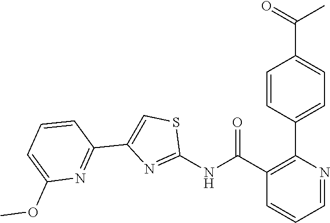 [SMILES: COc1cccc(-c2csc(NC(=O)c3cccnc3-c3ccc(C(C)=O)cc3)n2)n1] [symbol]
1870028-77-5 [symbol] 1261992-64 6 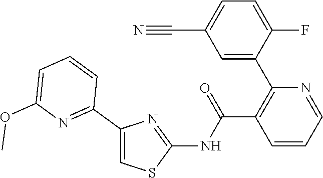 [SMILES: COc1cccc(-c2csc(NC(=O)c3cccnc3-c3cc(C#N)ccc3F)n2)n1] [symbol]
1870028-77-5 [symbol] 1261931-60-5 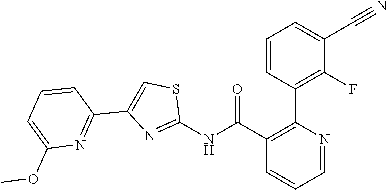 [SMILES: COc1cccc(-c2csc(NC(=O)c3cccnc3-c3cccc(C#N)c3F)n2)n1] [symbol]
1870028-77-5 [symbol] 1262005-32-2 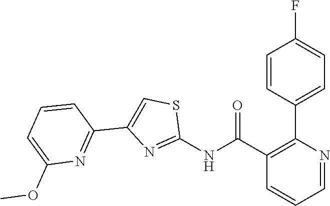 [SMILES: COc1cccc(-c2csc(NC(=O)c3cccnc3-c3ccc(F)cc3)n2)n1] [symbol]
1870028-77-5 [symbol] 101419-78-7 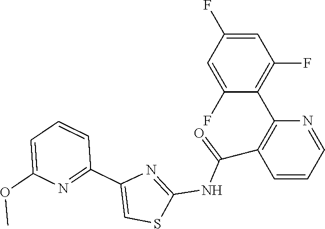 [SMILES: COc1cccc(-c2csc(NC(=O)c3cccnc3-c3c(F)cc(F)cc3F)n2)n1] [symbol]
1870028-77-5 [symbol] 1262000-75-8 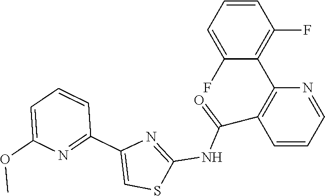 [SMILES: COc1cccc(-c2csc(NC(=O)c3cccnc3-c3c(F)cccc3F)n2)n1] [symbol]
1870028-77-5 [symbol] 1261954-54-4 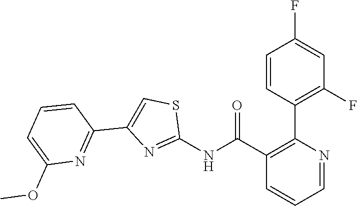 [SMILES: COc1cccc(-c2csc(NC(=O)c3cccnc3-c3ccc(F)cc3F)n2)n1] [symbol]
1870028-77-5 [symbol] 1258633-84-9 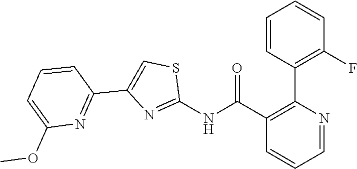 [SMILES: COc1cccc(-c2csc(NC(=O)c3cccnc3-c3ccccc3F)n2)n1] [symbol]
1870028-77-5 [symbol] 1214359-99-5 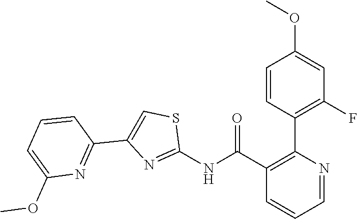 [SMILES: COc1ccc(-c2ncccc2C(=O)Nc2nc(-c3cccc(OC)n3)cs2)c(F)c1] [symbol]
1870028-77-5 [symbol] 1261898-93-4 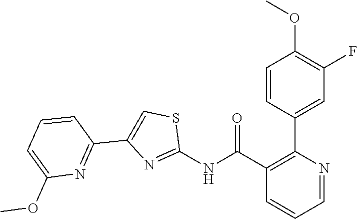 [SMILES: COc1cccc(-c2csc(NC(=O)c3cccnc3-c3ccc(OC)c(F)c3)n2)n1] [symbol]
1870028-77-5 [symbol] 1261922-79-5 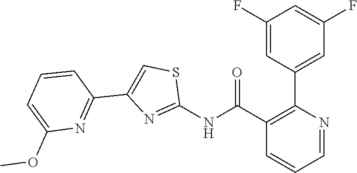 [SMILES: COc1cccc(-c2csc(NC(=O)c3cccnc3-c3cc(F)cc(F)c3)n2)n1] [symbol]
1870028-77-5 [symbol] 1225477-81-1 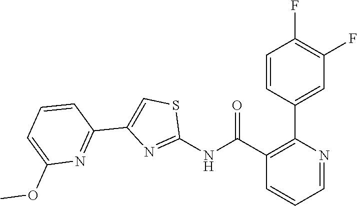 [SMILES: COc1cccc(-c2csc(NC(=O)c3cccnc3-c3ccc(F)c(F)c3)n2)n1] [symbol]
1870028-77-5 [symbol] 1225511-07-8 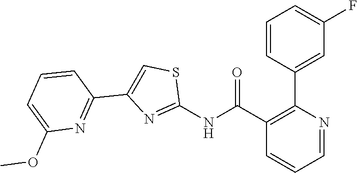 [SMILES: COc1cccc(-c2csc(NC(=O)c3cccnc3-c3cccc(F)c3)n2)n1] [symbol]
1870028-77-5 [symbol] 1214365-08-8 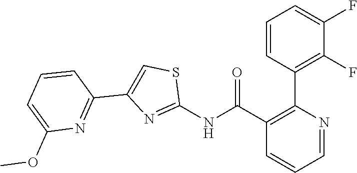 [SMILES: COc1cccc(-c2csc(NC(=O)c3cccnc3-c3cccc(F)c3F)n2)n1] [symbol]
1870028-77-5 [symbol] 1261504-55-5 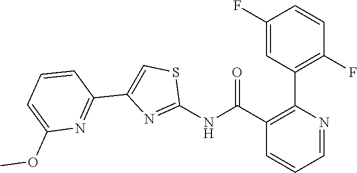 [SMILES: COc1cccc(-c2csc(NC(=O)c3cccnc3-c3cc(F)ccc3F)n2)n1] [symbol]
1870028-77-5 [symbol] 1261947-71-0 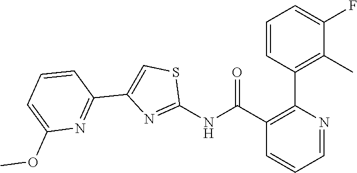 [symbol]
1870028-77-5 [symbol] 1261904-06-6 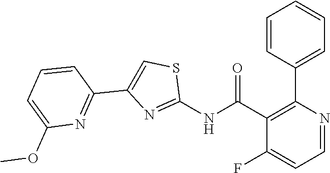 [SMILES: COc1cccc(-c2csc(NC(=O)c3c(F)ccnc3-c3ccccc3)n2)n1] [symbol]
1870028-77-5 [symbol] 1806489-43-9 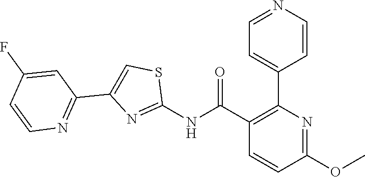 [SMILES: COc1ccc(C(=O)Nc2nc(-c3cc(F)ccn3)cs2)c(-c2ccncc2)n1] [symbol]
2228222-84-0 [symbol] 1607429-46-8 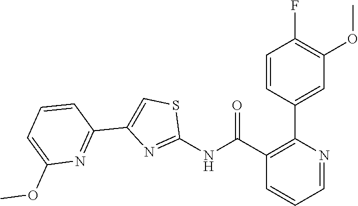 [symbol]
1870028-77-5 [symbol] 1261932-50-6 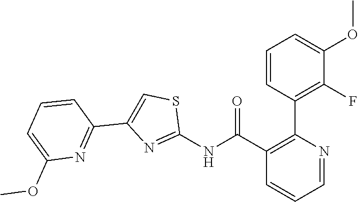 [SMILES: COc1cccc(-c2csc(NC(=O)c3cccnc3-c3cccc(OC)c3F)n2)n1] [symbol]
1870028-77-5 [symbol] 1261898-85-4 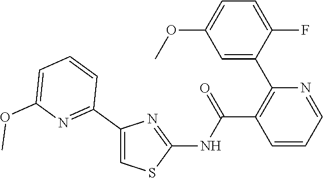 [SMILES: COc1ccc(F)c(-c2ncccc2C(=O)Nc2nc(-c3cccc(OC)n3)cs2)c1] [symbol]
1870028-77-5 [symbol] 1261999-97-6 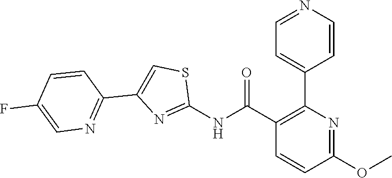 [SMILES: COc1ccc(C(=O)Nc2nc(-c3ccc(F)cn3)cs2)c(-c2ccncc2)n1] [symbol]
1551651-30-9 [symbol] 1607429-46-8 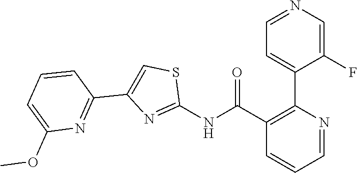 [SMILES: COc1cccc(-c2csc(NC(=O)c3cccnc3-c3ccncc3F)n2)n1] [symbol]
1870028-77-5 [symbol] 2092755-87-6 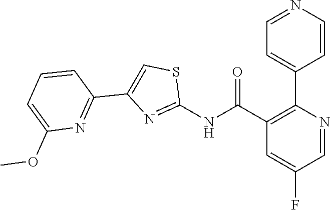 [SMILES: COc1cccc(-c2csc(NC(=O)c3cc(F)cnc3-c3ccncc3)n2)n1] [symbol]
1870028-77-5 [symbol] 1214360-97-8 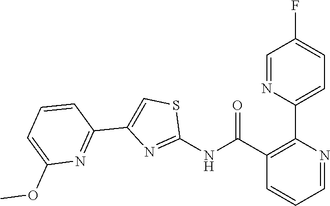 [SMILES: COc1cccc(-c2csc(NC(=O)c3cccnc3-c3ccc(F)cn3)n2)n1] [symbol]
1870028-77-5 [symbol] 2090618-35-0 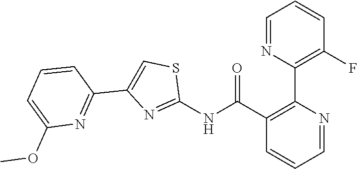 [SMILES: COc1cccc(-c2csc(NC(=O)c3cccnc3-c3ncccc3F)n2)n1] [symbol]
1870028-77-5 [symbol] 2091295-27-9 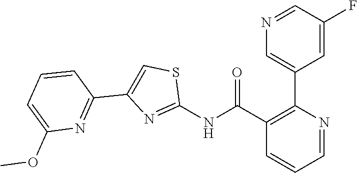 [SMILES: COc1cccc(-c2csc(NC(=O)c3cccnc3-c3cncc(F)c3)n2)n1] [symbol]
1870028-77-5 [symbol] 2092748-58-6 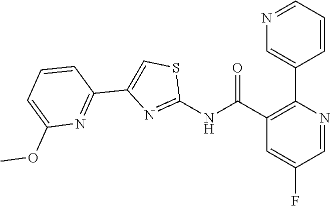 [SMILES: COc1cccc(-c2csc(NC(=O)c3cc(F)cnc3-c3cccnc3)n2)n1] [symbol]
1870028-77-5 [symbol] 1214335-84-8 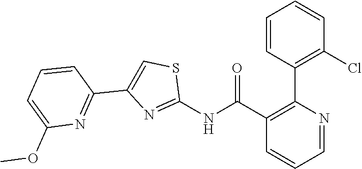 [SMILES: COc1cccc(-c2csc(NC(=O)c3cccnc3-c3ccccc3Cl)n2)n1] [symbol]
1870028-77-5 [symbol] 1226215-53-7 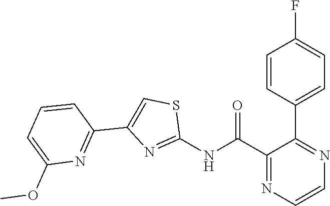 [SMILES: COc1cccc(-c2csc(NC(=O)c3nccnc3-c3ccc(F)cc3)n2)n1] [symbol]
1870028-77-5 [symbol] 1368895-24-2 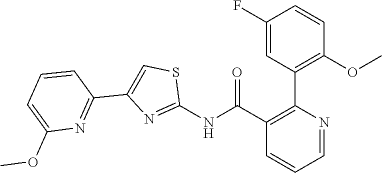 [symbol]
1870028-77-5 [symbol] 1255636-28-2 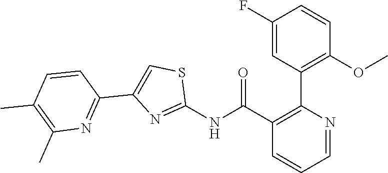 [symbol]
1934459-93-4 [symbol] 1255636-26-2 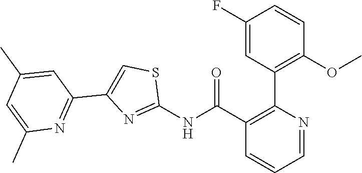 [symbol]
1865056-88-7 [symbol] 1255636-28-2 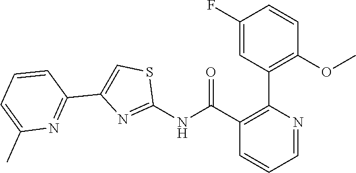 [SMILES: COc1ccc(F)cc1-c1ncccc1C(=O)Nc1nc(-c2cccc(C)n2)cs1] [symbol]
99358-99-3 [symbol] 1255636-28-2 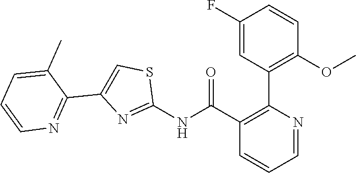 [SMILES: COc1ccc(F)cc1-c1ncccc1C(=O)Nc1nc(-c2ncccc2C)cs1] [symbol]
1044269-91-1 [symbol] 1255636-28-2 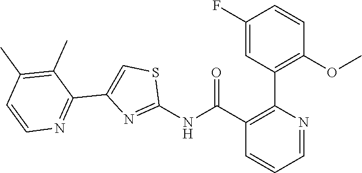 [symbol]
2169166-95-2 [symbol] 1255636-26-2 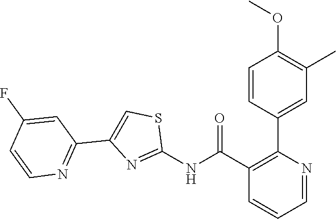 [SMILES: COc1ccc(-c2ncccc2C(=O)Nc2nc(-c3cc(F)ccn3)cs2)cc1C] [symbol]
2226222-84-0 [symbol] 1255634-54-8 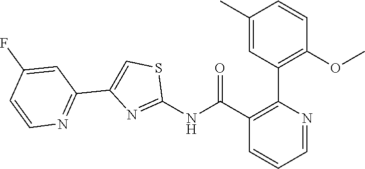 [SMILES: COc1ccc(C)cc1-c1ncccc1C(=O)Nc1nc(-c2cc(F)ccn2)cs1] [symbol]
2228222-84-0 [symbol] 1255638-89-1 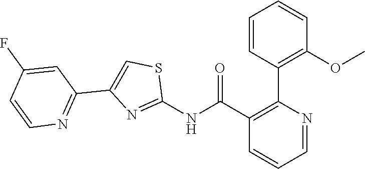 [SMILES: COc1ccccc1-c1ncccc1C(=O)Nc1nc(-c2cc(F)ccn2)cs1] [symbol]
2228222-84-0 [symbol] 1226261-41-1 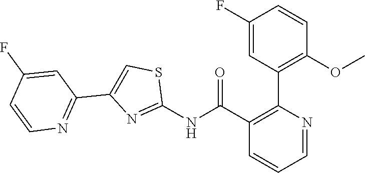 [SMILES: COc1ccc(F)cc1-c1ncccc1C(=O)Nc1nc(-c2cc(F)ccn2)cs1] [symbol]
2228222-84-0 [symbol] 1255636-28-2 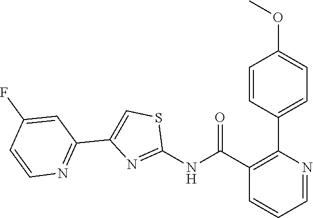 [SMILES: COc1ccc(-c2ncccc2C(=O)Nc2nc(-c3cc(F)ccn3)cs2)cc1] [symbol]
2228222-84-0 [symbol] 1225539-80-9 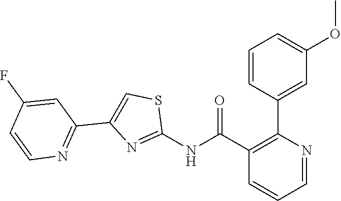 [SMILES: COc1cccc(-c2ncccc2C(=O)Nc2nc(-c3cc(F)ccn3)cs2)c1] [symbol]
2228222-84-0 [symbol] 912773-03-6 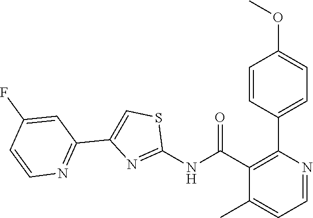 [SMILES: COc1ccc(-c2nccc(C)c2C(=O)Nc2nc(-c3cc(F)ccn3)cs2)cc1] [symbol]
2228222-84-0 [symbol] 956325-42-1 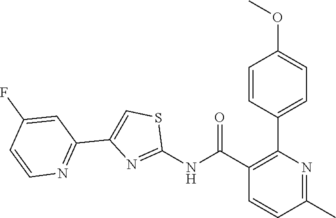 [SMILES: COc1ccc(-c2nc(C)ccc2C(=O)Nc2nc(-c3cc(F)ccn3)cs2)cc1] [symbol]
2228222-84-0 [symbol] 2322561-55-5 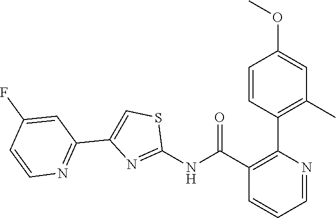 [SMILES: COc1ccc(-c2ncccc2C(=O)Nc2nc(-c3cc(F)ccn3)cs2)c(C)c1] [symbol]
2228222-84-0 [symbol] 1261912-94-0 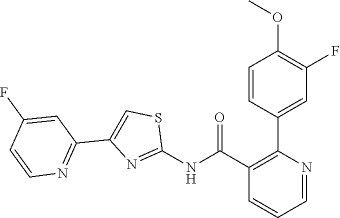 [SMILES: COc1ccc(-c2ncccc2C(=O)Nc2nc(-c3cc(F)ccn3)cs2)cc1F] [symbol]
2228222-84-0 [symbol] 1261922-79-5 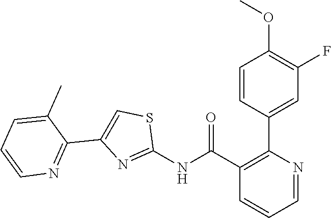 [SMILES: COc1ccc(-c2ncccc2C(=O)Nc2nc(-c3ncccc3C)cs2)cc1F] [symbol]
1044269-91-1 [symbol] 1261922-79-5 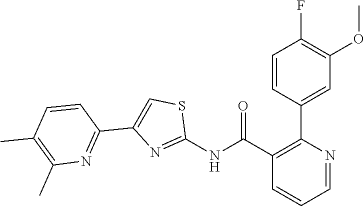 [symbol]
1934459-93-4 [symbol] 1261932-50-6 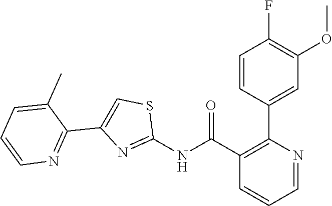 [SMILES: COc1cc(-c2ncccc2C(=O)Nc2nc(-c3ncccc3C)cs2)ccc1F] [symbol]
1044269-91-1 [symbol] 1261932-50-6 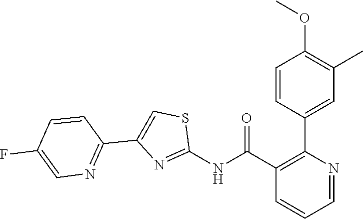 [SMILES: COc1ccc(-c2ncccc2C(=O)Nc2nc(-c3ccc(F)cn3)cs2)cc1C] [symbol]
1551651-30-9 [symbol] 1255634-54-8 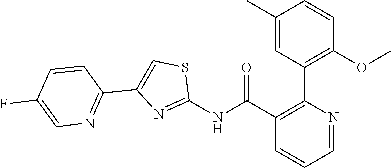 [SMILES: COc1ccc(C)cc1-c1ncccc1C(=O)Nc1nc(-c2ccc(F)cn2)cs1] [symbol]
1551651-30-9 [symbol] 1265638-89-1 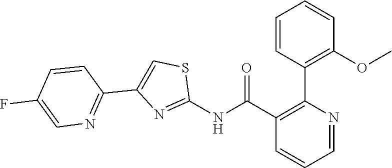 [SMILES: COc1ccccc1-c1ncccc1C(=O)Nc1nc(-c2ccc(F)cn2)cs1] [symbol]
1551851-30-9 [symbol] 1226281-41-1 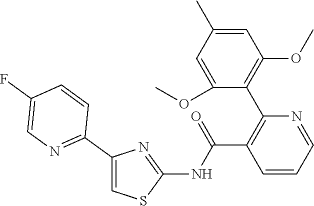 [SMILES: COc1cc(C)cc(OC)c1-c1ncccc1C(=O)Nc1nc(-c2ccc(F)cn2)cs1] [symbol]
1551651-30-9 [symbol] 2125607-71-6 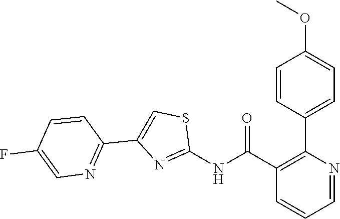 [SMILES: COc1ccc(-c2ncccc2C(=O)Nc2nc(-c3ccc(F)cn3)cs2)cc1] [symbol]
1551651-30-9 [symbol] 1225539-80-9 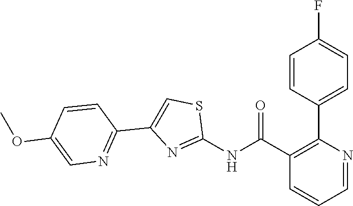 [SMILES: COc1ccc(-c2csc(NC(=O)c3cccnc3-c3ccc(F)cc3)n2)nc1] [symbol]
2228346-85-6 [symbol] 101419-78-7 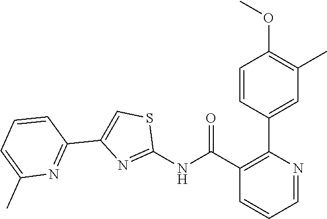 [SMILES: COc1ccc(-c2ncccc2C(=O)Nc2nc(-c3cccc(C)n3)cs2)cc1C] [symbol]
99358-99-3 [symbol] 1255634-54-8 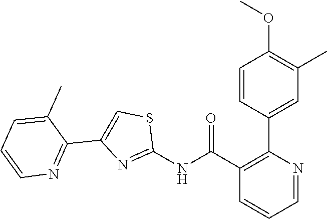 [SMILES: COc1ccc(-c2ncccc2C(=O)Nc2nc(-c3ncccc3C)cs2)cc1C] [symbol]
1044269-91-1 [symbol] 1255634-54-8 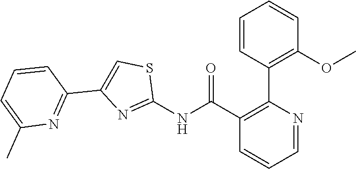 [SMILES: COc1ccccc1-c1ncccc1C(=O)Nc1nc(-c2cccc(C)n2)cs1] [symbol]
99358-99-3 [symbol] 1226261-41-1 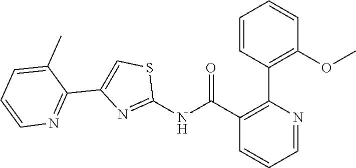 [SMILES: COc1ccccc1-c1ncccc1C(=O)Nc1nc(-c2ncccc2C)cs1] [symbol]
1044269-91-1 [symbol] 1226261-41-1 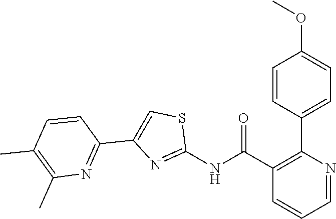 [SMILES: COc1ccc(-c2ncccc2C(=O)Nc2nc(-c3ccc(C)c(C)n3)cs2)cc1] [symbol]
1934459-93-4 [symbol] 1225539-80-9 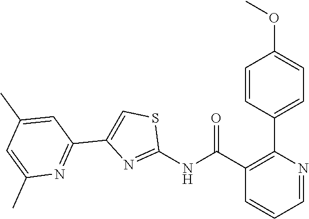 [SMILES: COc1ccc(-c2ncccc2C(=O)Nc2nc(-c3cc(C)cc(C)n3)cs2)cc1] [symbol]
1865056-88-7 [symbol] 1225539-80-9 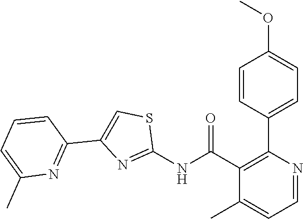 [SMILES: COc1ccc(-c2nccc(C)c2C(=O)Nc2nc(-c3cccc(C)n3)cs2)cc1] [symbol]
99358-99-3 [symbol] 956325-42-1 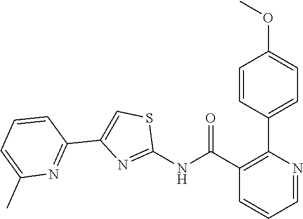 [SMILES: COc1ccc(-c2ncccc2C(=O)Nc2nc(-c3cccc(C)n3)cs2)cc1] [symbol]
99358-99-3 [symbol] 1225539-80-9 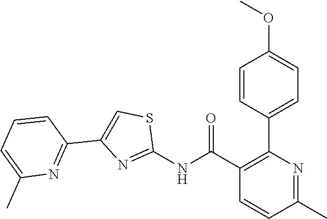 [SMILES: COc1ccc(-c2nc(C)ccc2C(=O)Nc2nc(-c3cccc(C)n3)cs2)cc1] [symbol]
99358-99-3 [symbol] 2322561-55-5 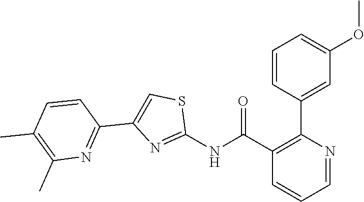 [symbol]
1934459-93-4 [symbol] 912773-03-6 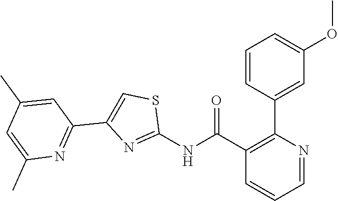 [symbol]
1865056-88-7 [symbol] 912773-03-6 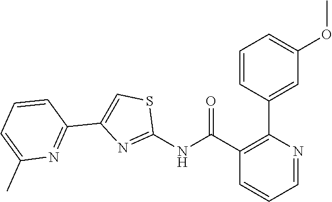 [SMILES: COc1cccc(-c2ncccc2C(=O)Nc2nc(-c3cccc(C)n3)cs2)c1] [symbol]
99358-99-3 [symbol] 912773-03-6 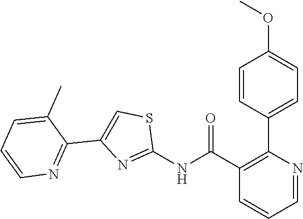 [SMILES: COc1ccc(-c2ncccc2C(=O)Nc2nc(-c3ncccc3C)cs2)cc1] [symbol]
1044269-91-1 [symbol] 1225539-80-9 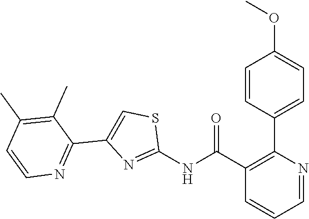 [SMILES: COc1ccc(-c2ncccc2C(=O)Nc2nc(-c3nccc(C)c3C)cs2)cc1] [symbol]
2169166-95-2 [symbol] 1225539-80-9 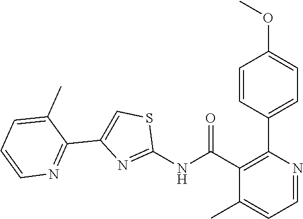 [SMILES: COc1ccc(-c2nccc(C)c2C(=O)Nc2nc(-c3ncccc3C)cs2)cc1] [symbol]
1044269-91-1 [symbol] 956325-42-1 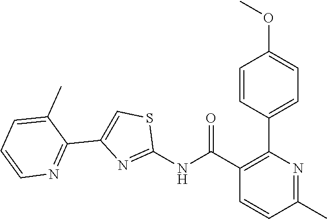 [SMILES: COc1ccc(-c2nc(C)ccc2C(=O)Nc2nc(-c3ncccc3C)cs2)cc1] [symbol]
1044269-91-1 [symbol] 2322561-55-5 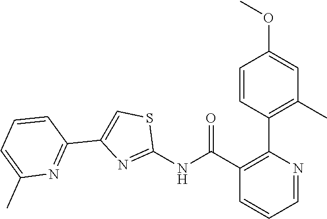 [SMILES: COc1ccc(-c2ncccc2C(=O)Nc2nc(-c3cccc(C)n3)cs2)c(C)c1] [symbol]
99358-99-3 [symbol] 1261912-94-0 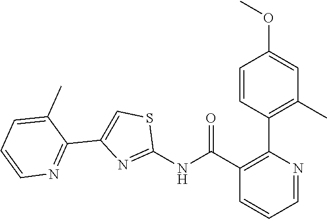 [SMILES: COc1ccc(-c2ncccc2C(=O)Nc2nc(-c3ncccc3C)cs2)c(C)c1] [symbol]
1044269-91-1 [symbol] 1261912-94-0 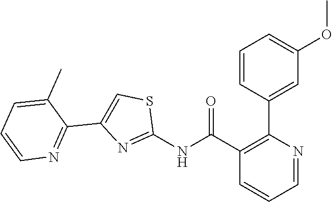 [SMILES: COc1cccc(-c2ncccc2C(=O)Nc2nc(-c3ncccc3C)cs2)c1] [symbol]
1044269-91-1 [symbol] 912773-03-6 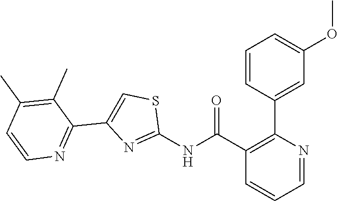 [symbol]
2169168-95-2 [symbol] 912773-03-8 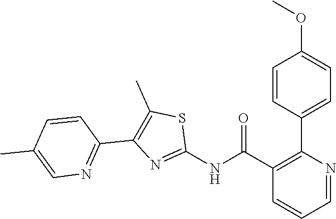 [SMILES: COc1ccc(-c2ncccc2C(=O)Nc2nc(-c3ccc(C)cn3)c(C)s2)cc1] [symbol]
2294598-48-6 [symbol] 1225539-80-9 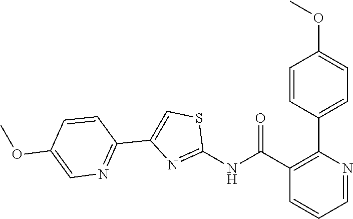 [SMILES: COc1ccc(-c2ncccc2C(=O)Nc2nc(-c3ccc(OC)cn3)cs2)cc1] [symbol]
2228346-85-6 [symbol] 1225539-80-9 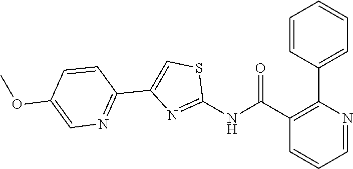 [SMILES: COc1ccc(-c2csc(NC(=O)c3cccnc3-c3ccccc3)n2)nc1] [symbol]
2228346-85-6 [symbol] 33421-39-5 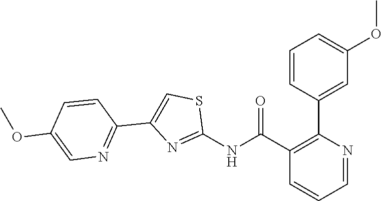 [symbol]
2228346-85-6 [symbol] 912773-03-6 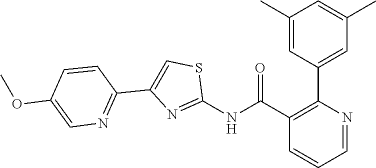 [symbol]
2228346-85-6 [symbol] 1226149-93-4 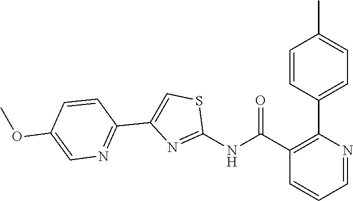 [SMILES: COc1ccc(-c2csc(NC(=O)c3cccnc3-c3ccc(C)cc3)n2)nc1] [symbol]
2228346-85-6 [symbol] 1226205-68-0 [symbol]
indicates data missing or illegible when filed

Activity in reporter assays

All compounds were tested using the human cell line HEK STF293. This cell line carries a Wnt reporter (TCF/LEF1 promoter), which drives expression of the firefly luciferase protein. The level of Wnt activity is directly correlated with the level of luciferase activity (determined using a simple assay). Compounds that inhibit Wnt signaling by reducing luciferase activity in these two cell lines are further tested biochemically. Biochemical confirmation that compounds inhibit Wnt signaling is obtained by immunoblotting for beta-catenin in HEK STF293 cells and demonstrating that its levels are reduced. Compounds were were further tested in a Viability assay using Wnt-dependent HCT116 cell line.

Compounds were prepared as 10 mM stocks for each compound in DMSO. Dilutions were prepared in a 96-well plate in DMSO. The stock dilutions are as follows: 10 mM, 1 mM, 100 μM, 10 μM, 100 nM, and 10 nM. Plates were sealed and stored at −20° C.

HEK STF293 cells were seeded at approximately 25,000-30,000 cells/well in a 96-well (100 μL volume). On the first day, Wnt3a-conditioned media (1:1) were added along with diluted compounds (1:100). For example, for 100 μL of STF293 cells, 100 μL of Wnt3a-conditioned media and 2 μL of drug was added to each well. The final concentrations should therefore be 100 μM, 10 μM, 1 μM, 100 nM, 10 nM, and 1 nM. On the second day, the media was removed and 75 μL of Passive Lysis Buffer (Promega) is added to each well. The plate was shaken at 130 rpm for 15 minutes. For the Steady Glo assay, 45 μL of the lysis was removed and added to a white 96-well plate containing 45 μL/well of Steady Glo solution (Promega). For the Cell Titer assay, 25 μL of the lysis was transferred to a white 96-well plate containing 25 μL/well of Cell Titer solution (Promega). Both Steady Glo and Cell Titer assays were read with a luminescence plate reader. When determining EC50, the Steady Glo values were divided by the Cell Titer values to normalize for cell number.

The control CMV driven cell line assay was performed as recited above for the STF293 assay except that no Wnt3a-conditioned media was added to the plated cells and 1 μL of diluted compound was added instead of 2 μL.

Three concentrations were chosen based on the EC50 curves from the STF293 assay. From the original 10 mM stocks, the following dilutions were prepared in DMSO and stored at −20° C.: 100 μM, 50 μM, and 10 μM.

HEK293 cells were seeded in a 6-well plate at approximately 8.0×10⁵ cells (2 mL per well). On the first day, Wnt3a-conditioned media (1:1) and compounds (1:100) were added to the plated cells. The final concentrations of compounds were 1 μM, 500 nM, and 100 nM. Vehicle (DMSO) and a Wnt3a-conditioned media plus Vehicle samples were also prepared as controls. Lysates were collected (with non-denaturing lysis buffer) after 24 hours incubation, and protein concentrations determined by Bradford Assay. Immunoblotting with an anti-beta-catenin antibody (equivalent amounts of protein/lane for each condition) were subsequently performed to determine beta-catenin levels. HCT116 cells were seeded at 2,500 cells/well in a 96-well dish (Volume: 100 μL/well). On the second day, the 10 mM stock solution of each test compound was thawed at room temperature. Dilutions of the test compound were prepared in DMSO in a V-bottom 96-well plate. The stock compound solution, DMSO-diluted compound solutions, and DMSO were further diluted 500-fold in cell culture media, and 100 μL of the compound- or DMSO-containing cell culture media was added to each well of the cells. The final compound concentrations, therefore, were 10 μM, 3.3.3 μM, 1.11 μM, 370.37 nM, 123.46 nM, 41.15 nM, 13.72 nM, 4.57 nM and zero. The final DMSO concentration was 0.1% in all wells. On the fifth day, 150 μL of media was removed from each well. 50 μL/well Cell Titer Glo reagent (Promega) was added to each well. The plate was shaken at 130 rpm for 15 minutes at room temperature, and the cell viability was determined using a luminescence plate reader.

Results from the HEK STF293 assay disclosed above are shown in Table 3 below. Results from the Viability assay disclosed above are shown in Table 9 below. Potency categories are defined as follows: A: <200 nM, B: 200-500 nM, C: 500-1000 nM, D: 1,000-10,000 nM.

TABLE 9

| Example number | TOPFlash IC₅₀ category | Viability IC₅₀ category |
|---|---|---|
| 1 | B | A |
| 2 | B | C |
| 3 | B | C |
| 4 | C | C |
| 5 | B | B |
| 6 | B | B |
| 7 | B | B |
| 8 | C | B |
| 9 | D | D |
| 10 | C | C |
| 11 | D | D |
| 12 | B | C |
| 13 | B | C |
| 14 | D | B |
| 15 | D | B |
| 16 | B | A |
| 17 | A | B |
| 18 | A | C |
| 19 | A | B |
| 20 | B | B |

What is claimed is:

1. A compound, or a pharmaceutically acceptable salt or tautomer thereof, having the formula: 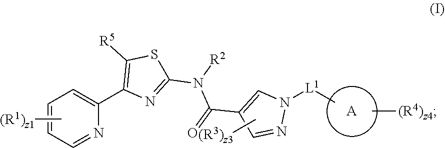
wherein
Ring A is cycloalkyl, heterocycloalkyl, aryl, or heteroaryl;
L¹ is a bond or substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NR^{1C}NR^{1A}R^{1B}, —ONR^{1A}R^{1B}, —NR^{1C}C(O)NR^{1A}R^{1B}—N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D} 10, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, , —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 2;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2;
wherein R³ is not —CF₃, unsubstituted methyl, unsubstituted ethyl, unsubstituted cyclopropyl, substituted or unsubstituted phenyl, unsubstituted pyrrolyl, or unsubstituted thienyl; and
wherein when Ring A is phenyl, then z3 is not 0.

2. A compound, or a pharmaceutically acceptable salt or tautomer thereof, having the formula: 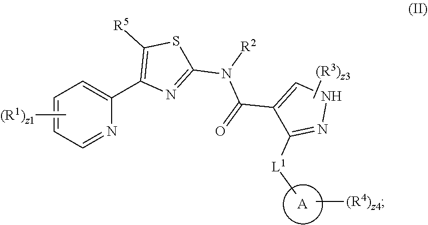
wherein
Ring A is cycloalkyl, heterocycloalkyl, aryl, or heteroaryl;
L¹ is a bond or substituted or unsubstituted alkylene;
R¹ is independently halogen, —CX¹₃, —CHX¹₂, —CH₂X¹, —OCX¹₃, —OCH₂X¹, —OCHX¹₂, —CN, —SOᵥ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}—N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 2;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2;
wherein Ring A is not cyclopropyl or pyrrolyl;
wherein R³ is not unsubstituted phenyl;
wherein when Ring A is phenyl, then R⁴ is not unsubstituted methoxy; and
wherein when Ring A is phenyl, then z3 is not 0.

3. A compound, or a pharmaceutically acceptable salt or tautomer thereof, having the formula: 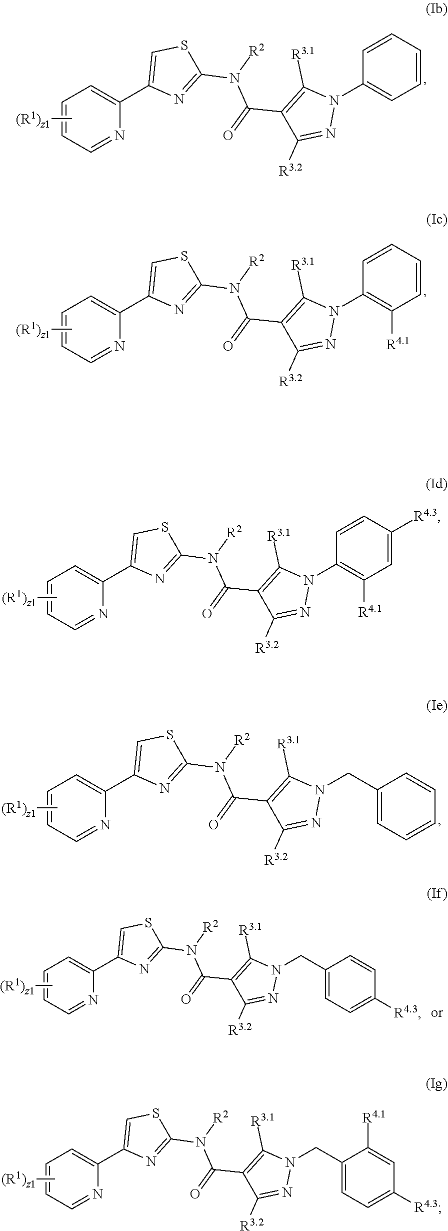
R¹ is independently halogen, —CX¹₃, —CHX¹₂, —CH₂X¹, —OCX¹₃, —OCH₂X¹, —OCHX¹₂, —CN, —SOᵥ₁R^{1D}, —SOᵥ₁NR^{1A}R^{1B}, —NR^{1C}NR^{1A}R^{1B}, —ONR^{1A}R^{1B}, —N^{1C}C(O)NR^{1A}R^{1B}—N(O)ₘ₁, —NR^{1A}R^{1B}, C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{3.1} and R^{3.2} are independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D} , —NR^{3A}SO₂R^{3D}, —NR^{3A}C(O)R^{3C}, —NR^{3A}C(O)OR^{3C}, —NR^{3A}OR^{3C}—SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{4.1}and R^{4.3} are independently halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}—ONR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D} —NR^{4A}SO₂R^{4D}, —NR^{4A}C(O)R^{4C}, —NR^{4A}C(O)OR^{4C}, —NR^{4A}OR^{4C}—SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2.

4. A compound, or a pharmaceutically acceptable salt or tautomer thereof, having the formula:

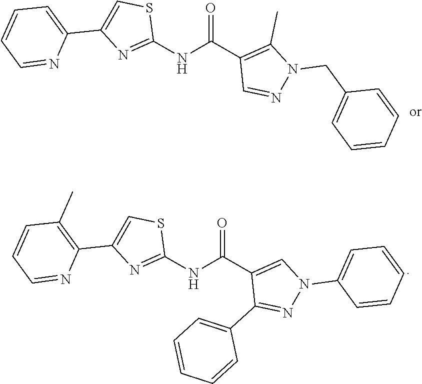 [SMILES: Cc1c(C(=O)Nc2nc(-c3ccccn3)cs2)cnn1Cc1ccccc1 ; Cc1cccnc1-c1csc(NC(=O)c2cn(-c3ccccc3)nc2-c2ccccc2)n1]

5. A compound, or a pharmaceutically acceptable salt or tautomer thereof, having the formula: 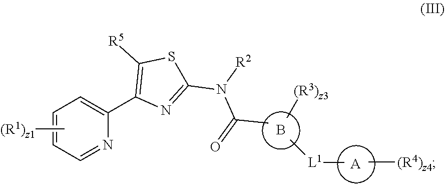
wherein
Ring A is cycloalkyl, heterocycloalkyl, aryl, or heteroaryl;
Ring B is imidazolyl or triazolyl;
L¹ is a bond or substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}—ONR^{1A}R^{1B}—NR^{1C}C(O)NR^{1A}R^{1B}—N(O)ₘ₁, —NR^{1A}R^{1B}, —C(O)R^{1C}, —C(O)OR^{1C}, —C(O)NR^{1A}R^{1B}, —OR^{1D}-NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is an integer from 0 to 2;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D}—NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2;
wherein when Ring A is phenyl, then z4 is not 0.

6. The compound of claim 5, having the formula:

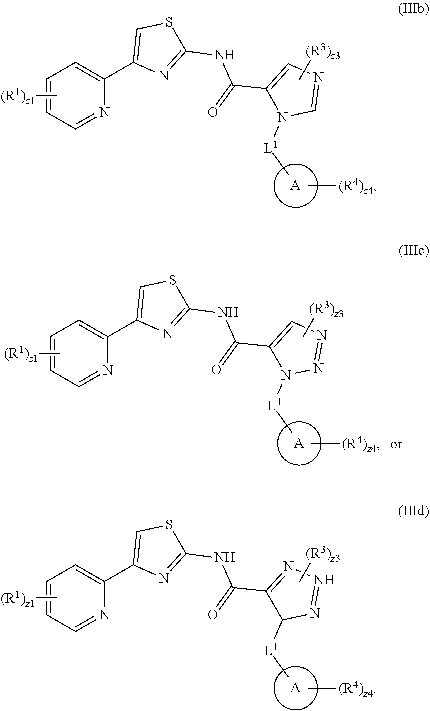

7. A pharmaceutical composition comprising a pharmaceutically acceptable excipient and a compound, or a pharmaceutically acceptable salt or tautomer thereof, having the formula: 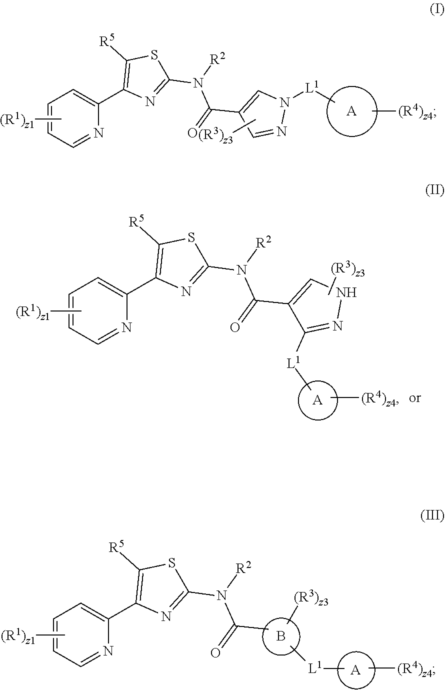
wherein
Ring A is cycloalkyl, heterocycloalkyl, aryl, or heteroaryl;
Ring B is imidazolyl or triazolyl;
L¹ is a bond or substituted or unsubstituted alkylene;
R¹ is independently halogen, —CX¹₃, —CHX¹₂, —CH₂X¹, —OCX¹₃, —OCH₂X¹, —OCHX¹₂, —CN, —SOᵥ₁R^{1D}, —SOᵥ₁NR^{1A}R^{1B}, —NR^{1C}NR^{1A}R^{1B}, —ONR^{1A}R^{1B}—NR^{1C}C(O)NR^{1A}R^{1B}, —N(O)ₘ₁, —NR^{1A}R^{1B}, —C(O)R^{1C}, —C(O)OR^{1C}, C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —Soᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 2;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}-N⁴CNR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D}—NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2.

8. A method of treating a cancer in a subject in need thereof, said method comprising administering to the subject a therapeutically effective amount of a compound, or a pharmaceutically acceptable salt or tautomer thereof, having the formula: 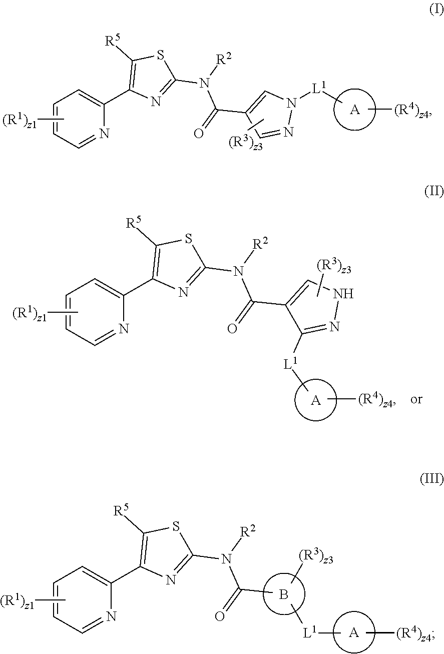
wherein
Ring A is cycloalkyl, heterocycloalkyl, aryl, or heteroaryl;
Ring B is imidazolyl or triazolyl;
L¹ is a bond or substituted or unsubstituted alkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}—ONR^{1A}R^{1B}, —NR^{1C}C(O)NR^{1A}R^{1B}—N(O)ₘ₁, —NR^{1A}R^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}—NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 2;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —N⁴CNR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D}—NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2.

9. A compound, or a pharmaceutically acceptable salt or tautomer thereof, having the formula: 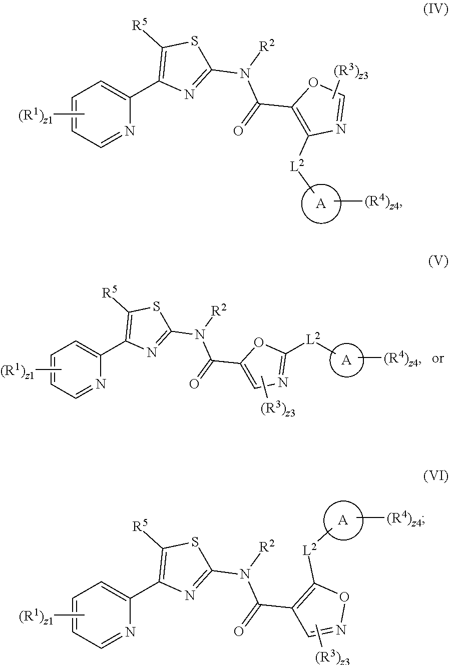
wherein
Ring A is cycloalkyl, heterocycloalkyl, aryl, or heteroaryl;
L² is a bond, —C(O)NR¹⁰—, —NR¹⁰C(O)—, —NR¹⁰S(O)₂—, —S(O)₂NR¹⁰—, substituted or unsubstituted alkylene, or substituted or unsubstituted heteroalkylene;
R¹⁰ is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}—ONR^{1A}R^{1B}—NR^{1C}C(O)NR^{1A}R^{1B}—N(O)ₘ₁, —NR^{1A}R^{1B}, —C(O)R^{1C}, —C(O)OR^{1C}, —C(O)NR^{1A}R^{1B}, —OR^{1D}-_{NR}^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
32 R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}—NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is 0 or 1;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR⁴C, —C(O)NR^{4A}R^{4B}, —OR^{4D}, —NR⁴ASO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2.

10. A compound, or a pharmaceutically acceptable salt or tautomer thereof, having the formula: 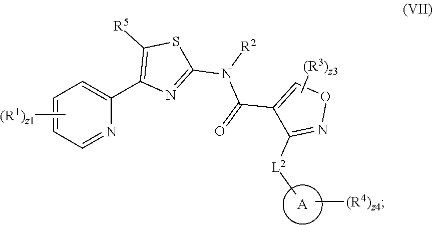
wherein
Ring A is cycloalkyl, heterocycloalkyl, aryl, or heteroaryl;
L² is a bond, —C(O)NR¹⁰—, —NR¹⁰C(O)—, —NR¹⁰S(O)₂—, —S(O)₂NR¹⁰—, substituted or unsubstituted alkylene, or substituted or unsubstituted heteroalkylene;
R¹⁰ is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOᵥ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}—N(O)ₘ₁, —NR 1AR^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is 0 or 1;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2;
wherein when Ring A is phenyl or 4-pyridyl, then z4 is not 0;
wherein when Ring A is phenyl, then at least one R⁴ is not —Cl; and
wherein when Ring A is phenyl and z4 is 1, then R⁴ is not —CH₃.

11. A compound, or a pharmaceutically acceptable salt or tautomer thereof, having the formula:

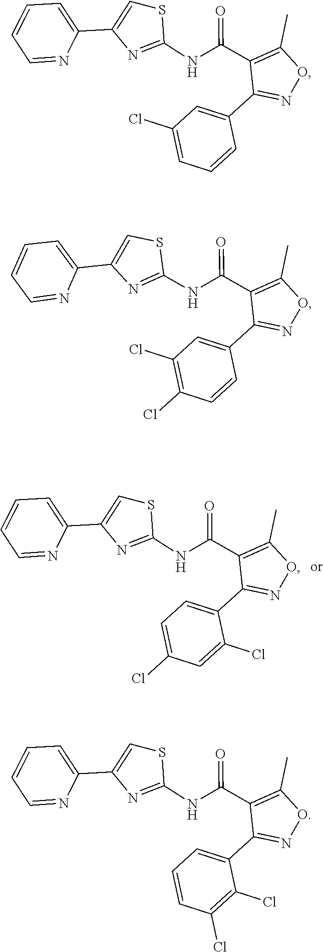 [SMILES: Cc1onc(-c2cccc(Cl)c2)c1C(=O)Nc1nc(-c2ccccn2)cs1 ; Cc1onc(-c2ccc(Cl)c(Cl)c2)c1C(=O)Nc1nc(-c2ccccn2)cs1 ; Cc1onc(-c2ccc(Cl)cc2Cl)c1C(=O)Nc1nc(-c2ccccn2)cs1 ; Cc1onc(-c2cccc(Cl)c2Cl)c1C(=O)Nc1nc(-c2ccccn2)cs1]

12. A pharmaceutical composition comprising a pharmaceutically acceptable excipient and a compound, or a pharmaceutically acceptable salt or tautomer thereof, having the formula: 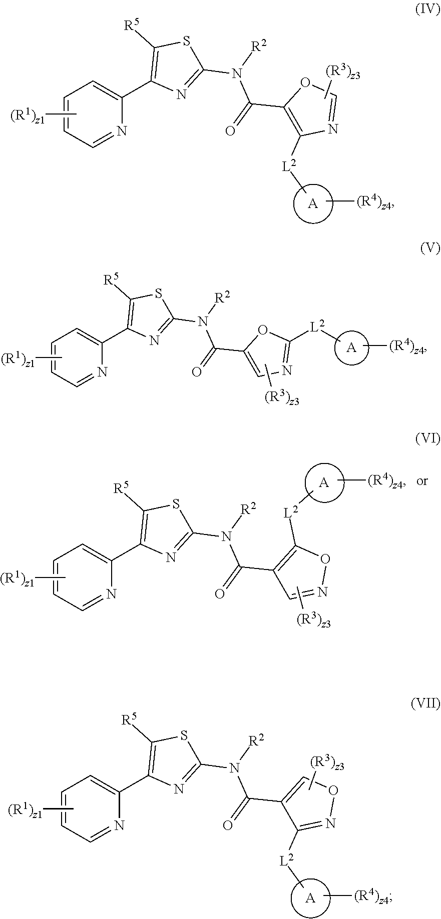
wherein
Ring A is cycloalkyl, heterocycloalkyl, aryl, or heteroaryl;
L² is a bond, —C(O)NR¹⁰—, —NR¹⁰C(O)—, —NR¹⁰S(O)₂—, —S(O)₂NR¹⁰—, substituted or unsubstituted alkylene, or substituted or unsubstituted heteroalkylene;
R¹⁰ is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}—ONR^{1A}R^{1B}—NR^{1C}C(O)NR^{1A}R^{1B}—N(O)ₘ₁, —NR 1AR^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is 0 or 1;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D} —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2.

13. A method of treating a cancer in a subject in need thereof, said method comprising administering to the subject a therapeutically effective amount of a compound, or a pharmaceutically acceptable salt or tautomer thereof, having the formula: 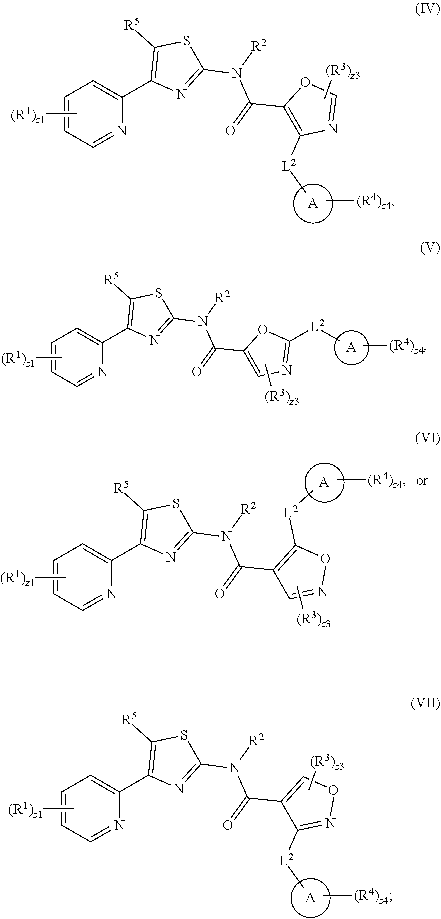
wherein
Ring A is cycloalkyl, heterocycloalkyl, aryl, or heteroaryl;
L² is a bond, —C(O)NR¹⁰—, —NR¹⁰C(O)—, —NR¹⁰S(O)₂—, —S(O)₂NR¹⁰—, substituted or unsubstituted alkylene, or substituted or unsubstituted heteroalkylene;
R¹⁰ is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}—ONR^{1A}R^{1B}—NR^{1C}C(O)NR^{1A}R^{1B}—N(O)ₘ₁, —NR 1AR^{1B}, —C(O)R^{1C}, —C(O)OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z3 is 0 or 1;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D} —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2.

14. A compound, or a pharmaceutically acceptable salt or tautomer thereof, having the formula: 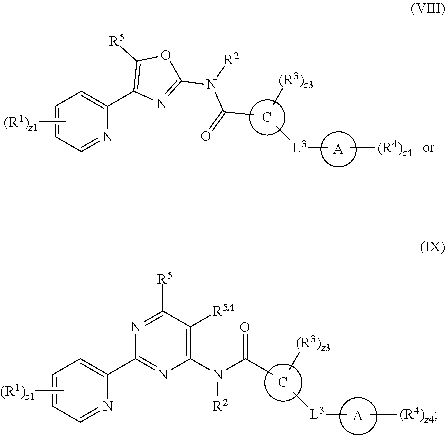
wherein
Ring A is cycloalkyl, heterocycloalkyl, aryl, or heteroaryl;
Ring C is pyrazolyl, oxazolyl, pyrrolyl, imidazolyl, triazolyl, or tetrazolyl;
L³ is a bond, —O—, substituted or unsubstituted alkylene, or substituted or unsubstituted heteroalkylene;
R¹ is independently halogen, —CX¹₃, —CHX¹₂, —CH₂X¹, —OCX¹₃, —OCH₂X¹, —OCHX¹₂, —CN, —SOᵥ₁R^{1D}, —SOᵥ₁NR^{1A}R^{1B}, —NR^{1C}NR^{1A}R^{1B}, —ONR^{1A}R^{1B}—NR^{1C}C(O)NR^{1A}R^{1B}—N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —Soᵥ₃NR^{3A}R^{3B}, —NR^{3C}NR^{3A}R^{3B}—ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 2;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR⁴D —NR⁴ASO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ and R^{5A} are independently hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁵ substituents may optionally be joined to form a substituted or unsubstituted unsubstituted aryl or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2.

15. The compound of claim 14, having the formula:

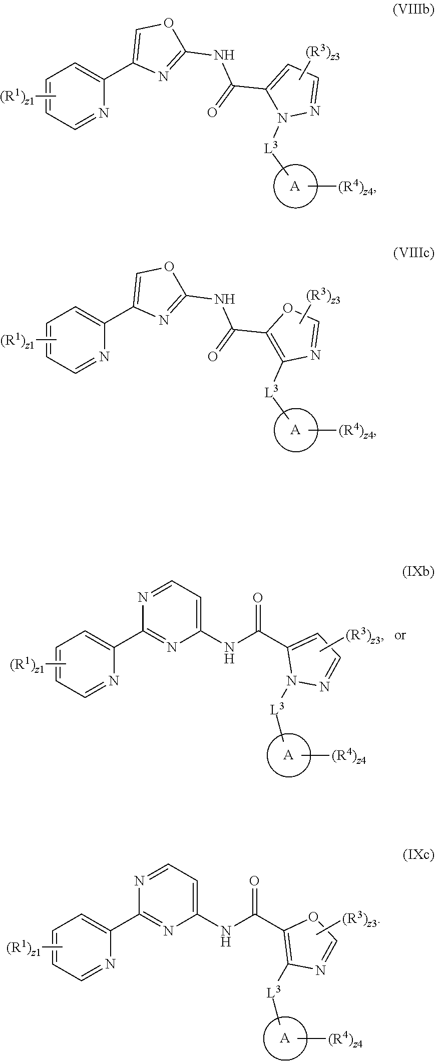

16. A pharmaceutical composition comprising the compound of claim 14, or a pharmaceutically acceptable salt or tautomer thereof, and a pharmaceutically acceptable excipient.

17. A method of treating a cancer in a subject in need thereof, said method comprising administering to the subject a therapeutically effective amount of the compound of claim 14, or a pharmaceutically acceptable salt or tautomer thereof.

18. A compound, or a pharmaceutically acceptable salt or tautomer thereof, having the formula: 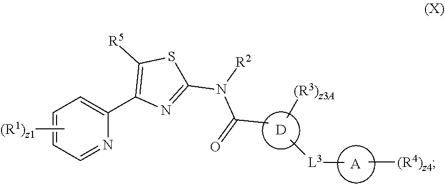
wherein
Ring A is cycloalkyl, heterocycloalkyl, aryl, or heteroaryl;
Ring D is pyridonyl, pyrazinyl, pyridyl, or phenyl;
L³ is a bond, —O—, substituted or unsubstituted alkylene, or substituted or unsubstituted heteroalkylene;
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}—ONR^{1A}R^{1B}, —NR^{1C}C(O)NR^{1A}R^{1B}—N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 4;
R² is hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, ONR^{3A}R^{3B}, —NR^{3C}C(O)NR^{3A}R^{3B}—N(O)ₘ₃, —NR^{3A}R^{3B}, —C(O)R^{3C}, —C(O)OR^{3C}, —C(O)NR^{3A}R^{3B}, —OR^{3D}, —NR³ASO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3A is an integer from 0 to 4;
R⁴ is independently oxo, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}—ONR^{4A}R^{4B}, —NR^{4C}C(O)NR^{4A}R^{4B}—N(O)ₘ₄, —NR^{4A}R^{4B}, —C(O)R^{4C}, —C(O)OR^{4C}, —C(O)NR^{4A}R^{4B}, —OR^{4D}, —NR⁴ASO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁴ substituents may optionally be joined to form a substituted or unsubstituted cycloalkyl, substituted or unsubstituted heterocycloalkyl, substituted or unsubstituted aryl, or substituted or unsubstituted heteroaryl;
z4 is an integer from 0 to 11;
R⁵ is hydrogen, halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{3A}, R^{3B}, R^{3C}, R^{3D}, R^{4A}, R^{4B}, R^{4C}, and R^{4D} are independently hydrogen, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
each X¹, X³, and X⁴ is independently —F, —Cl, —Br, or —I;
n1, n3, and n4 are independently an integer from 0 to 4; and
m1, m3, m4, v1, v3, and v4 are independently 1 or 2.

19. A pharmaceutical composition comprising the compound of claim 18, or a pharmaceutically acceptable salt or tautomer thereof, and a pharmaceutically acceptable excipient.

20. A method of treating a cancer in a subject in need thereof, said method comprising administering to the subject a therapeutically effective amount of the compound of claim 18, or a pharmaceutically acceptable salt or tautomer thereof.
